SEQUENCE LISTING (693 sequences)

SEQ ID NO: 1 - protein, 120 aa, Homo sapiens
  REGION 1..120: note = ADT1-4 Variable Heavy
QVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWSGYVDVWGQGTLVTVSS

SEQ ID NO: 2 - protein, 120 aa, Homo sapiens
  REGION 1..120: note = ADT1-4-105 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDVWGQGTLVTVSS

SEQ ID NO: 3 - protein, 120 aa, Homo sapiens
  REGION 1..120: note = ADT1-4-107 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSS

SEQ ID NO: 4 - protein, 120 aa, Homo sapiens
  REGION 1..120: note = ADT1-4-110 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVEYVDVWGQGTLVTVSS

SEQ ID NO: 5 - protein, 120 aa, Homo sapiens
  REGION 1..120: note = ADT1-4-112 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDYWGQGTLVTVSS

SEQ ID NO: 6 - protein, 120 aa, Homo sapiens; identical to SEQ ID NO: 2
  REGION 1..120: note = ADT1-4-117 Variable Heavy

SEQ ID NO: 7 - protein, 120 aa, Homo sapiens
  REGION 1..120: note = ADT1-4-19 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSS

SEQ ID NO: 8 - protein, 120 aa, Homo sapiens
  REGION 1..120: note = ADT1-4-21 Variable Heavy
EVQLQQSGPGLVKPSQTLSLTCAISGDSVSSKSVAWNWIRQSPSRGLEWLGRTYYRSKWSTDYAASVKSRITINPDTSKNQLSLQLNSVTPEDTAVYYCARTWADYVDVWGQGTLVTVSS

SEQ ID NO: 9 - protein, 120 aa, Homo sapiens; identical to SEQ ID NO: 3
  REGION 1..120: note = ADT1-4-31 Variable Heavy

SEQ ID NO: 10 - protein, 120 aa, Homo sapiens; identical to SEQ ID NO: 5
  REGION 1..120: note = ADT1-4-139 Variable Heavy

SEQ ID NO: 11 - protein, 120 aa, Homo sapiens; identical to SEQ ID NO: 3
  REGION 1..120: note = ADT1-4-4 Variable Heavy

SEQ ID NO: 12 - protein, 120 aa, Homo sapiens
  REGION 1..120: note = ADT1-4-143 Variable Heavy
EVQLLQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSS

SEQ ID NO: 13 - protein, 120 aa, Homo sapiens; identical to SEQ ID NO: 8
  REGION 1..120: note = ADT1-4-53 Variable Heavy

SEQ ID NO: 14 - protein, 120 aa, Homo sapiens
  REGION 1..120: note = ADT1-4-173 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWAGYPDVWGQGTLVTVSS

SEQ ID NO: 15 - protein, 120 aa, Homo sapiens; identical to SEQ ID NO: 7
  REGION 1..120: note = ADT1-4-2 Variable Heavy

SEQ ID NO: 16 - protein, 120 aa, Homo sapiens
  REGION 1..120: note = ADT1-4-8 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARSWVGYVDVWGQGTLVTVSS

SEQ ID NO: 17 - protein, 120 aa, Homo sapiens; identical to SEQ ID NO: 16
  REGION 1..120: note = ADT1-4-82 Variable Heavy

SEQ ID NO: 18 - protein, 120 aa, Homo sapiens; identical to SEQ ID NO: 16
  REGION 1..120: note = ADT1-4-83 Variable Heavy

SEQ ID NO: 19 - protein, 120 aa, Homo sapiens
  REGION 1..120: note = ADT1-4-3 Variable Heavy
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWADYVDVWGQGTLVTVSS

SEQ ID NO: 20 - protein, 120 aa, Homo sapiens; identical to SEQ ID NO: 3
  REGION 1..120: note = ADT1-4-84 Variable Heavy

SEQ ID NO: 21 - protein, 120 aa, Homo sapiens
  REGION 1..120: note = ADT1-4-86 Variable Heavy
EVQLQQSGPGLVKPSQTLSLTCAISGDSVSSKSVAWNWIRQSPSRGLEWLGRTYYRSKWSTDYAASVKSRITINPDTSKNQLSLQLNSVTPEDTAVYYCARTWLGNVDVWGQGTLVTVSS

SEQ ID NO: 22 - protein, 120 aa, Homo sapiens; identical to SEQ ID NO: 3
  REGION 1..120: note = ADT1-4-95 Variable Heavy

SEQ ID NO: 23 - protein, 120 aa, Homo sapiens; identical to SEQ ID NO: 3
  REGION 1..120: note = ADT1-4-1 Variable Heavy

SEQ ID NO: 24 - protein, 120 aa, Homo sapiens; identical to SEQ ID NO: 14
  REGION 1..120: note = ADT1-4-6 Variable Heavy

SEQ ID NO: 25 - protein, 120 aa, Homo sapiens; identical to SEQ ID NO: 19
  REGION 1..120: note = ADT1-4-138 Variable Heavy

SEQ ID NO: 26 - protein, 110 aa, Homo sapiens
  REGION 1..110: note = ADT1-4 Variable Light
ASDIQMTQSPPALSASVGDRVTITCRASQDINDWLAWYQHKPGKAPKLLIYDASSLESGVPSRFSGSGSGTEFTLTISSLQPDDFATYYCQQSYSTPQVTFGQGTRLEIK

SEQ ID NO: 27 - protein, 108 aa, Homo sapiens
  REGION 1..108: note = ADT1-4-105 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPQITFGQGTRLEIK

SEQ ID NO: 28 - protein, 108 aa, Homo sapiens
  REGION 1..108: note = ADT1-4-107 Variable Light
DIQMTQSPPALSASVGDRVTITCRASQDINDWLAWYQHKPGKAPKLLIYDASSLESGVPLRFSGSGSGTEFTLTISSLQPDDFATYYCQQRYSTPQITFGQGTRLEIK

SEQ ID NO: 29 - protein, 108 aa, Homo sapiens
  REGION 1..108: note = ADT1-4-110 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPQVTFGAGTRLEIK

SEQ ID NO: 30 - protein, 108 aa, Homo sapiens
  REGION 1..108: note = ADT1-4-112 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSQPQVTFGQGTRLEIK

SEQ ID NO: 31 - protein, 108 aa, Homo sapiens
  REGION 1..108: note = ADT1-4-117 Variable Light
DIQMTQSPPALSASVGDRVTITCRASQDINDWLAWYQHKPGKAPKLLIYDASSLESGVPLRFSGSGSGTEFTLTISSLQPDDFATYYCQQRYSTPRVTFGQGTRLEIK

SEQ ID NO: 32 - protein, 108 aa, Homo sapiens
  REGION 1..108: note = ADT1-4-19 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPKVTFGQGTRLEIK

SEQ ID NO: 33 - protein, 108 aa, Homo sapiens
  REGION 1..108: note = ADT1-4-21 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPWVTFGQGTRLEIK

SEQ ID NO: 34 - protein, 108 aa, Homo sapiens
  REGION 1..108: note = ADT1-4-31 Variable Light
DIQMTQSPPALSASVGDRVTITCRASQDINDWLAWYQHKPGKAPKLLIYDASSLESGVPLRFSGSGSGTEFTLTISSLQPDDFATYYCQQRYSTPPVTFGQGTRLEIK

SEQ ID NO: 35 - protein, 108 aa, Homo sapiens
  REGION 1..108: note = ADT1-4-139 Variable Light
DIQMTQSPPALSASVGDRVTITCRASQDINDWLAWYQHKPGKAPKLLIYDASSLESGVPLRFSGSGSGTEFTLTISSLQPDDFATYYCQQGYSTPQLTFGQGTRLEIK

SEQ ID NO: 36 - protein, 108 aa, Homo sapiens; identical to SEQ ID NO: 33
  REGION 1..108: note = ADT1-4-4 Variable Light

SEQ ID NO: 37 - protein, 108 aa, Homo sapiens
  REGION 1..108: note = ADT1-4-143 Variable Light
DIQMTQSPPALSASVGDRVTITCRASQDINDWLAWYQHKPGKAPKLLIYDASSLESGVPLRFSGSGSGTEFTLTISSLQPDDFATYYCQQRYSTHQVTFGQGTRLVIK

SEQ ID NO: 38 - protein, 108 aa, Homo sapiens
  REGION 1..108: note = ADT1-4-53 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPQLTFGQGTRLEIK

SEQ ID NO: 39 - protein, 108 aa, Homo sapiens
  REGION 1..108: note = ADT1-4-173 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIK

SEQ ID NO: 40 - protein, 108 aa, Homo sapiens; identical to SEQ ID NO: 39
  REGION 1..108: note = ADT1-4-2 Variable Light

SEQ ID NO: 41 - protein, 108 aa, Homo sapiens; identical to SEQ ID NO: 39
  REGION 1..108: note = ADT1-4-8 Variable Light

SEQ ID NO: 42 - protein, 108 aa, Homo sapiens; identical to SEQ ID NO: 28
  REGION 1..108: note = ADT1-4-82 Variable Light

SEQ ID NO: 43 - protein, 108 aa, Homo sapiens
  REGION 1..108: note = ADT1-4-83 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSEPQVTFGQGTRLEIK

SEQ ID NO: 44 - protein, 108 aa, Homo sapiens
  REGION 1..108: note = ADT1-4-3 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPEVTFGQGTRLEIK

SEQ ID NO: 45 - protein, 108 aa, Homo sapiens
  REGION 1..108: note = ADT1-4-84 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSDPQVTFGQGTRLEIK

SEQ ID NO: 46 - protein, 108 aa, Homo sapiens; identical to SEQ ID NO: 43
  REGION 1..108: note = ADT1-4-86 Variable Light

SEQ ID NO: 47 - protein, 108 aa, Homo sapiens
  REGION 1..108: note = ADT1-4-95 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYSTPQVTFGSGTRLEIK

SEQ ID NO: 48 - protein, 108 aa, Homo sapiens
  REGION 1..108: note = ADT1-4-1 Variable Light
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIK

SEQ ID NO: 49 - protein, 108 aa, Homo sapiens
  REGION 1..108: note = ADT1-4-6 Variable Light
DIQMTQSPPALSASVGDRVTITCRASQDINDWLAWYQHKPGKAPKLLIYDASSLESGVPLRFSGSGSGTEFTLTISSLQPDDFATYYCQQKYKTPQVTFGQGTRLEIK

SEQ ID NO: 50 - protein, 108 aa, Homo sapiens
  REGION 1..108: note = ADT1-4-138 Variable Light
DIQMTQSPPALSASVGDRVTITCRASQDINDWLAWYQHKPGKAPKLLIYDASSLESGVPSRFSGSGSGTEFTLTISSLQPDDFATYYCQQRYSTDQVTFGQGTRLEIK

SEQ ID NO: 51 - protein, 10 aa, Homo sapiens
  REGION 1..10: note = ADT1-4 VHCDR1 (parental and affinity matured clones)
GDSVSSKSAA

SEQ ID NO: 52 - protein, 10 aa, Homo sapiens
  REGION 1..10: note = ADT1-4-21/ADT1-4-53/ADT1-4-86 VHCDR1
GDSVSSKSVA

SEQ ID NO: 53 - protein, 9 aa, Homo sapiens
  REGION 1..9: note = ADT1-4 VHCDR2 (parental and all affinity matured clones)
TYYRSKWST

SEQ ID NO: 54 - protein, 8 aa, Homo sapiens
  REGION 1..8: note = ADT1-4 VHCDR3
TWSGYVDV

SEQ ID NO: 55 - protein, 8 aa, Homo sapiens
  REGION 1..8: note = ADT1-4-105 VHCDR3
TWVGYVDV

SEQ ID NO: 56 - protein, 8 aa, Homo sapiens
  REGION 1..8: note = ADT1-4-107 VHCDR3
TWVGYADV

SEQ ID NO: 57 - protein, 8 aa, Homo sapiens
  REGION 1..8: note = ADT1-4-110 VHCDR3
TWVEYVDV

SEQ ID NO: 58 - protein, 8 aa, Homo sapiens
  REGION 1..8: note = ADT1-4-112 VHCDR3
TWVGYVDY

SEQ ID NO: 59 - protein, 8 aa, Homo sapiens
  REGION 1..8: note = ADT1-4-117 VHCDR3
TWVGYVDV

SEQ ID NO: 60 - protein, 8 aa, Homo sapiens
  REGION 1..8: note = ADT1-4-19 VHCDR3
TWVGYVDR

SEQ ID NO: 61 - protein, 8 aa, Homo sapiens
  REGION 1..8: note = ADT1-4-21 VHCDR3
TWADYVDV

SEQ ID NO: 62 - protein, 8 aa, Homo sapiens
  REGION 1..8: note = ADT1-4-31 VHCDR3
TWVGYADV

SEQ ID NO: 63 - protein, 8 aa, Homo sapiens
  REGION 1..8: note = ADT1-4-139 VHCDR3
TWVGYVDY

SEQ ID NO: 64 - protein, 8 aa, Homo sapiens
  REGION 1..8: note = ADT1-4-4 VHCDR3
TWVGYADV

SEQ ID NO: 65 - protein, 8 aa, Homo sapiens
  REGION 1..8: note = ADT1-4-143 VHCDR3
TWVGYADV

SEQ ID NO: 66 - protein, 8 aa, Homo sapiens
  REGION 1..8: note = ADT1-4-53 VHCDR3
TWADYVDV

SEQ ID NO: 67 - protein, 8 aa, Homo sapiens
  REGION 1..8: note = ADT1-4-173 VHCDR3
TWAGYPDV

SEQ ID NO: 68 - protein, 8 aa, Homo sapiens
  REGION 1..8: note = ADT1-4-2 VHCDR3
TWVGYVDR

SEQ ID NO: 69 - protein, 8 aa, Homo sapiens
  REGION 1..8: note = ADT1-4-8 VHCDR3
TWVGYADV

SEQ ID NO: 70 - protein, 8 aa, Homo sapiens
  REGION 1..8: note = ADT1-4-82 VHCDR3
SWVGYVDV

SEQ ID NO: 71 - protein, 8 aa, Homo sapiens
  REGION 1..8: note = ADT1-4-83 VHCDR3
SWVGYVDV

SEQ ID NO: 72 - protein, 8 aa, Homo sapiens
  REGION 1..8: note = ADT1-4-3 VHCDR3
TWADYVDV

SEQ ID NO: 73 - protein, 8 aa, Homo sapiens
  REGION 1..8: note = ADT1-4-84 VHCDR3
TWVGYADV

SEQ ID NO: 74 - protein, 8 aa, Homo sapiens
  REGION 1..8: note = ADT1-4-86 VHCDR3
TWLGNVDV

SEQ ID NO: 75 - protein, 8 aa, Homo sapiens
  REGION 1..8: note = ADT1-4-95 VHCDR3
TWVGYADV

SEQ ID NO: 76 - protein, 8 aa, Homo sapiens
  REGION 1..8: note = ADT1-4-1 VHCDR3
TWVGYADV

SEQ ID NO: 77 - protein, 8 aa, Homo sapiens
  REGION 1..8: note = ADT1-4-6 VHCDR3
TWAGYPDV

SEQ ID NO: 78 - protein, 8 aa, Homo sapiens
  REGION 1..8: note = ADT1-4-138 VHCDR3
TWADYVDV

SEQ ID NO: 79 - protein, 6 aa, Homo sapiens
  REGION 1..6: note = ADT1-4 VLCDR1 (parental and all affinity matured clones)
QDINDW

SEQ ID NO: 80 - skipped

SEQ ID NO: 81 - protein, 10 aa, Homo sapiens
  REGION 1..10: note = ADT1-4 VLCDR3
QQSYSTPQVT

SEQ ID NO: 82 - protein, 10 aa, Homo sapiens
  REGION 1..10: note = ADT1-4-105 VLCDR3
QQKYSTPQIT

SEQ ID NO: 83 - protein, 10 aa, Homo sapiens
  REGION 1..10: note = ADT1-4-107 VLCDR3
QQRYSTPQIT

SEQ ID NO: 84 - protein, 10 aa, Homo sapiens
  REGION 1..10: note = ADT1-4-110 VLCDR3
QQKYSTPQVT

SEQ ID NO: 85 - protein, 10 aa, Homo sapiens
  REGION 1..10: note = ADT1-4-112 VLCDR3
QQKYSQPQVT

SEQ ID NO: 86 - protein, 10 aa, Homo sapiens
  REGION 1..10: note = ADT1-4-117 VLCDR3
QQRYSTPRVT

SEQ ID NO: 87 - protein, 10 aa, Homo sapiens
  REGION 1..10: note = ADT1-4-19 VLCDR3
QQKYSTPKVT

SEQ ID NO: 88 - protein, 10 aa, Homo sapiens
  REGION 1..10: note = ADT1-4-21 VLCDR3
QQKYSTPWVT

SEQ ID NO: 89 - protein, 10 aa, Homo sapiens
  REGION 1..10: note = ADT1-4-31 VLCDR3
QQRYSTPPVT

SEQ ID NO: 90 - protein, 10 aa, Homo sapiens
  REGION 1..10: note = ADT1-4-139 VLCDR3
QQGYSTPQLT

SEQ ID NO: 91 - protein, 10 aa, Homo sapiens
  REGION 1..10: note = ADT1-4-4 VLCDR3
QQKYSTPWVT

SEQ ID NO: 92 - protein, 10 aa, Homo sapiens
  REGION 1..10: note = ADT1-4-143 VLCDR3
QQRYSTHQVT

SEQ ID NO: 93 - protein, 10 aa, Homo sapiens
  REGION 1..10: note = ADT1-4-53 VLCDR3
QQKYSTPQLT

SEQ ID NO: 94 - protein, 10 aa, Homo sapiens
  REGION 1..10: note = ADT1-4-173 VLCDR3
QQKYSAPQVT

SEQ ID NO: 95 - protein, 10 aa, Homo sapiens
  REGION 1..10: note = ADT1-4-2 VLCDR3
QQKYSAPQVT

SEQ ID NO: 96 - protein, 10 aa, Homo sapiens
  REGION 1..10: note = ADT1-4-8 VLCDR3
QQKYSAPQVT

SEQ ID NO: 97 - protein, 10 aa, Homo sapiens
  REGION 1..10: note = ADT1-4-82 VLCDR3
QQRYSTPQIT

SEQ ID NO: 98 - protein, 10 aa, Homo sapiens
  REGION 1..10: note = ADT1-4-83 VLCDR3
QQKYSEPQVT

SEQ ID NO: 99 - protein, 10 aa, Homo sapiens
  REGION 1..10: note = ADT1-4-3 VLCDR3
QQKYSTPEVT

SEQ ID NO: 100 - protein, 10 aa, Homo sapiens
  REGION 1..10: note = ADT1-4-84 VLCDR3
QQKYSDPQVT

SEQ ID NO: 101 - protein, 10 aa, Homo sapiens
  REGION 1..10: note = ADT1-4-86 VLCDR3
QQKYSEPQVT

SEQ ID NO: 102 - protein, 10 aa, Homo sapiens
  REGION 1..10: note = ADT1-4-95 VLCDR3
QQKYSTPQVT

SEQ ID NO: 103 - protein, 10 aa, Homo sapiens
  REGION 1..10: note = ADT1-4-1 VLCDR3
QQRYSTPIVT

SEQ ID NO: 104 - protein, 10 aa, Homo sapiens
  REGION 1..10: note = ADT1-4-6 VLCDR3
QQKYKTPQVT

SEQ ID NO: 105 - protein, 10 aa, Homo sapiens
  REGION 1..10: note = ADT1-4-138 VLCDR3
QQRYSTDQVT

SEQ ID NO: 106 - protein, 118 aa, Homo sapiens
  REGION 1..118: note = ADT1-7 Variable Heavy
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVDYADAFDIWGQGTLVTVSS

SEQ ID NO: 107 - protein, 118 aa, Homo sapiens
  REGION 1..118: note = ADT1-7-10 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDDYNDAFDIWGQGTLVTVSS

SEQ ID NO: 108 - protein, 118 aa, Homo sapiens; identical to SEQ ID NO: 107
  REGION 1..118: note = ADT1-7-15 Variable Heavy

SEQ ID NO: 109 - protein, 118 aa, Homo sapiens
  REGION 1..118: note = ADT1-7-17 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARIDYEDAFDIWGQGTLVTVSS

SEQ ID NO: 110 - protein, 118 aa, Homo sapiens
  REGION 1..118: note = ADT1-7-18 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVSYDDAFDIWGQGTLVTVSS

SEQ ID NO: 111 - protein, 118 aa, Homo sapiens; identical to SEQ ID NO: 109
  REGION 1..118: note = ADT1-7-19 Variable Heavy

SEQ ID NO: 112 - protein, 118 aa, Homo sapiens
  REGION 1..118: note = ADT1-7-20 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVDYQEAFDIWGQGTLVTVSS

SEQ ID NO: 113 - protein, 118 aa, Homo sapiens; identical to SEQ ID NO: 107
  REGION 1..118: note = ADT1-7-22 Variable Heavy

SEQ ID NO: 114 - protein, 118 aa, Homo sapiens
  REGION 1..118: note = ADT1-7-23 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVDYNEAFDIWGQGTLVTVSS

SEQ ID NO: 115 - protein, 118 aa, Homo sapiens
  REGION 1..118: note = ADT1-7-42 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDDYEDAFDIWGQGTLVTVSS

SEQ ID NO: 116 - protein, 118 aa, Homo sapiens
  REGION 1..118: note = ADT1-7-3 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVSYAEAFDIWGQGTLVTVSS

SEQ ID NO: 117 - protein, 118 aa, Homo sapiens
  REGION 1..118: note = ADT1-7-61 Variable Heavy
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDDYDDAFDIWGQGTLVTVSS

SEQ ID NO: 118 - protein, 109 aa, Homo sapiens
  REGION 1..109: note = ADT1-7 Variable Light
ASDIQMTQSPSSLSASVGDRVTIACRAGQSIGTYLNWYQQKPGKAPKLLIYVASSLQSGVPSRFSGSGSGTEFTLTISSLQPEDFATYYCQQSYSTLLTFGRGTKVEIK

SEQ ID NO: 119 - protein, 107 aa, Homo sapiens
  REGION 1..107: note = ADT1-7-10 Variable Light
DIQMTQSPSSLSASVGDRVTIACRAGQSIGTYLNWYQQKPGKAPKLLIYVASSLQSGVPSRFSGSGSGTEFTLTISSLQPEDFATYYCQQTASTLLTFGRGTKVEIK

SEQ ID NO: 120 - protein, 107 aa, Homo sapiens
  REGION 1..107: note = ADT1-7-15 Variable Light
DIQMTQSPSSLSASVGDRVTIACRAGQSIGTYLNWYQQKPGKAPKLLIYVASSLQSGVPSRFSGSGSGTEFTLTISSLQPEDFATYYCQQSADTLLTFGRGTKVEIK

SEQ ID NO: 121 - protein, 107 aa, Homo sapiens
  REGION 1..107: note = ADT1-7-17 Variable Light
DIQMTQSPSSLSASVGDRVTIACRAGQSIGTYLNWYQQKPGKAPKLLIYVASSLQSGVPSRFSGSGSGTEFTLTISSLQPEDFATYYCQQSGSELLTFGRGTKVEIK

SEQ ID NO: 122 - protein, 107 aa, Homo sapiens
  REGION 1..107: note = ADT1-7-18 Variable Light
DIQMTQSPSSLSASVGDRVTIACRVGQSIGTYLNWYQQKPGKAPKLLIYVASSLQSGVPSRFSGSGSGTEFTLTISSLQPEDFATYYCQQSASELLTFGRGTKVEIK

SEQ ID NO: 123 - protein, 107 aa, Homo sapiens
  REGION 1..107: note = ADT1-7-19 Variable Light
DIQMTQSPSSLSASVGDRVTIACRAGQSIGTYLNWYQQKPGKAPKLLIYVASSLQSGVPSRFSGSGSGTEFTLTISSLQPEDFATYYCQQSASELLTFGRGTKVEIK

SEQ ID NO: 124 - protein, 107 aa, Homo sapiens
  REGION 1..107: note = ADT1-7-20 Variable Light
DIQMTQSPSSLSASVGDRVTIACRAGQSIGTYLNWYQQKPGKAPKLLIYVASSLQSGVPSRFSGSGSGTEFTLTISSLQPEDFATYYCQQSYSGLDTFGRGTKVEIK

SEQ ID NO: 125 - protein, 107 aa, Homo sapiens; identical to SEQ ID NO: 123
  REGION 1..107: note = ADT1-7-22 Variable Light

SEQ ID NO: 126 - protein, 107 aa, Homo sapiens; identical to SEQ ID NO: 124
  REGION 1..107: note = ADT1-7-23 Variable Light

SEQ ID NO: 127 - protein, 107 aa, Homo sapiens
  REGION 1..107: note = ADT1-7-42 Variable Light
DIQMTQSPSSLSASVGDRVTIACRAGQSIGTYLNWYQQKPGKAPKLLIYVASSLQSGVPSRFSGSGSGTEFTLTISSLQPEDFATYYCQQSSSELLTFGRGTKVEIK

SEQ ID NO: 128 - protein, 107 aa, Homo sapiens; identical to SEQ ID NO: 124
  REGION 1..107: note = ADT1-7-3 Variable Light

SEQ ID NO: 129 - protein, 107 aa, Homo sapiens; identical to SEQ ID NO: 123
  REGION 1..107: note = ADT1-7-61 Variable Light

SEQ ID NO: 130 - protein, 8 aa, Homo sapiens
  REGION 1..8: note = ADT1-7 VHCDR1 (parental and all affinity matured clones)
GFTFSDYY

SEQ ID NO: 131 - protein, 8 aa, Homo sapiens
  REGION 1..8: note = ADT1-7 VHCDR2 (parental and all affinity matured clones)
ISSSGSTI

SEQ ID NO: 132 - protein, 9 aa, Homo sapiens
  REGION 1..9: note = ADT1-7 VHCDR3
VDYADAFDI

SEQ ID NO: 133 - protein, 9 aa, Homo sapiens
  REGION 1..9: note = ADT1-7-10 VHCDR3
DDYNDAFDI

SEQ ID NO: 134 - protein, 9 aa, Homo sapiens
  REGION 1..9: note = ADT1-7-15 VHCDR3
DDYNDAFDI

SEQ ID NO: 135 - protein, 9 aa, Homo sapiens
  REGION 1..9: note = ADT1-7-17 VHCDR3
IDYEDAFDI

SEQ ID NO: 136 - protein, 9 aa, Homo sapiens
  REGION 1..9: note = ADT1-7-18 VHCDR3
VSYDDAFDI

SEQ ID NO: 137 - protein, 9 aa, Homo sapiens
  REGION 1..9: note = ADT1-7-19 VHCDR3
IDYEDAFDI

SEQ ID NO: 138 - protein, 9 aa, Homo sapiens
  REGION 1..9: note = ADT1-7-20 VHCDR3
VDYQEAFDI

SEQ ID NO: 139 - protein, 9 aa, Homo sapiens
  REGION 1..9: note = ADT1-7-22 VHCDR3
DDYNDAFDI

SEQ ID NO: 140 - protein, 9 aa, Homo sapiens
  REGION 1..9: note = ADT1-7-23 VHCDR3
VDYNEAFDI

SEQ ID NO: 141 - protein, 9 aa, Homo sapiens
  REGION 1..9: note = ADT1-7-42 VHCDR3
DDYEDAFDI

SEQ ID NO: 142 - protein, 9 aa, Homo sapiens
  REGION 1..9: note = ADT1-7-3 VHCDR3
VSYAEAFDI

SEQ ID NO: 143 - protein, 9 aa, Homo sapiens
  REGION 1..9: note = ADT1-7-61 VHCDR3
DDYDDAFDI

SEQ ID NO: 144 - protein, 6 aa, Homo sapiens
  REGION 1..6: note = ADT1-7 VLCDR1 (parental and all affinity matured clones)
QSIGTY

SEQ ID NO: 145 - skipped

SEQ ID NO: 146 - protein, 9 aa, Homo sapiens
  REGION 1..9: note = ADT1-7 VLCDR3
QQSYSTLLT

SEQ ID NO: 147 - protein, 9 aa, Homo sapiens
  REGION 1..9: note = ADT1-7-10 VLCDR3
QQTASTLLT

SEQ ID NO: 148 - protein, 9 aa, Homo sapiens
  REGION 1..9: note = ADT1-7-15 VLCDR3
QQSADTLLT

SEQ ID NO: 149 - protein, 9 aa, Homo sapiens
  REGION 1..9: note = ADT1-7-17 VLCDR3
QQSGSELLT

SEQ ID NO: 150 - protein, 9 aa, Homo sapiens
  REGION 1..9: note = ADT1-7-18 VLCDR3
QQSASELLT

SEQ ID NO: 151 - protein, 9 aa, Homo sapiens
  REGION 1..9: note = ADT1-7-19 VLCDR3
QQSASELLT

SEQ ID NO: 152 - protein, 9 aa, Homo sapiens
  REGION 1..9: note = ADT1-7-20 VLCDR3
QQSYSGLDT

SEQ ID NO: 153 - protein, 9 aa, Homo sapiens
  REGION 1..9: note = ADT1-7-22 VLCDR3
QQSASELLT

SEQ ID NO: 154 - protein, 9 aa, Homo sapiens
  REGION 1..9: note = ADT1-7-23 VLCDR3
QQSYSGLDT

SEQ ID NO: 155 - protein, 9 aa, Homo sapiens
  REGION 1..9: note = ADT1-7-42 VLCDR3
QQSSSELLT

SEQ ID NO: 156 - protein, 9 aa, Homo sapiens
  REGION 1..9: note = ADT1-7-3 VLCDR3
QQSYSGLDT

SEQ ID NO: 157 - protein, 9 aa, Homo sapiens
  REGION 1..9: note = ADT1-7-61 VLCDR3
QQSASELLT

SEQ ID NO: 158 - protein, 10 aa, Homo sapiens
  REGION 1..10: note = ADT1-4-derived anti-TCR delta variable 1 antibody or antigen-binding fragment thereof: HCDR1
  VARIANT 9: note = X can be any naturally occurring amino acid
GDSVSSKSXA

SEQ ID NO: 159 - protein, 10 aa, Homo sapiens
  REGION 1..10: note = ADT1-4-derived anti-TCR delta variable 1 antibody or antigen-binding fragment thereof: HCDR1
  VARIANT 9: note = X is A or V
GDSVSSKSXA

SEQ ID NO: 160 - protein, 10 aa, Homo sapiens
  REGION 1..10: note = ADT1-4-derived anti-TCR delta variable 1 antibody or antigen-binding fragment thereof: HCDR1
  VARIANT 9: note = X is A or V
GDSVSSKSXA

SEQ ID NO: 161 - protein, 10 aa, Homo sapiens
  REGION 1..10: note = ADT1-4-derived anti-TCR delta variable 1 antibody or antigen-binding fragment thereof: HCDR1
  VARIANT 9: note = X is A or V
GDSVSSKSXA

SEQ ID NO: 162-165 - skipped (4 entries)

SEQ ID NO: 166 - protein, 10 aa, Homo sapiens
  REGION 1..10: note = ADT1-4-derived anti-TCR delta variable 1 antibody or antigen-binding fragment thereof: LCDR3
  VARIANT 3: note = X can be any naturally occurring amino acid
  VARIANT 5..9: note = X can be any naturally occurring amino acid
QQXYXXXXXT

SEQ ID NO: 167 - protein, 10 aa, Homo sapiens
  REGION 1..10: note = ADT1-4-derived anti-TCR delta variable 1 antibody or antigen-binding fragment thereof: LCDR3
  VARIANT 3: note = X is K, R or G
  VARIANT 5: note = X is S or K
  VARIANT 6: note = X is T, Q, A, E or D
  VARIANT 7: note = X is P, H or D
  VARIANT 8: note = X is Q, R, K, W, P, E or I
  VARIANT 9: note = X is I, V or L
QQXYXXXXXT

SEQ ID NO: 168 - protein, 10 aa, Homo sapiens
  REGION 1..10: note = ADT1-4-derived anti-TCR delta variable 1 antibody or antigen-binding fragment thereof: LCDR3
  VARIANT 3: note = X is K, R or G
  VARIANT 5: note = X is S or K
  VARIANT 6: note = X is T, Q, A, E or D
  VARIANT 7: note = X is P or H
  VARIANT 8: note = X is Q, R, K, W, P, E or I
  VARIANT 9: note = X is I, V or L
QQXYXXXXXT

SEQ ID NO: 169 - protein, 10 aa, Homo sapiens
  REGION 1..10: note = ADT1-4-derived anti-TCR delta variable 1 antibody or antigen-binding fragment thereof: LCDR3
  VARIANT 3: note = X is K or R
  VARIANT 5: note = X is S or K
  VARIANT 6: note = X is T, Q, A or E
  VARIANT 7: note = X is P or H
  VARIANT 8: note = X is Q, K, W, P or I
  VARIANT 9: note = X is V or L
QQXYXXXXXT

SEQ ID NO: 170 - protein, 25 aa, Homo sapiens
  REGION 1..25: note = ADT1-4 HFR1 (parental and affinity matured clones)
EVQLQQSGPGLVKPSQTLSLTCAIS

SEQ ID NO: 171 - protein, 25 aa, Homo sapiens
  REGION 1..25: note = ADT1-4-143 HFR1
EVQLLQSGPGLVKPSQTLSLTCAIS

SEQ ID NO: 172 - protein, 17 aa, Homo sapiens
  REGION 1..17: note = ADT1-4 HFR2 (parental and all affinity matured clones)
WNWIRQSPSRGLEWLGR

SEQ ID NO: 173 - protein, 40 aa, Homo sapiens
  REGION 1..40: note = ADT1-4 HFR3 (parental and all affinity matured clones)
DYAASVKSRITINPDTSKNQLSLQLNSVTPEDTAVYYCAR

SEQ ID NO: 174 - protein, 11 aa, Homo sapiens
  REGION 1..11: note = ADT1-4 HFR4 (parental and all affinity matured clones)
WGQGTLVTVSS

SEQ ID NO: 175 - protein, 26 aa, Homo sapiens
  REGION 1..26: note = ADT1-4 LFR1 (parental and all affinity matured clones)
DIQMTQSPPALSASVGDRVTITCRAS

SEQ ID NO: 176 - protein, 17 aa, Homo sapiens
  REGION 1..17: note = ADT1-4 LFR2 (parental and all affinity matured clones)
LAWYQHKPGKAPKLLIY

SEQ ID NO: 177 - protein, 36 aa, Homo sapiens
  REGION 1..36: note = ADT1-4 LFR3 (affinity matured clones)
SLESGVPLRFSGSGSGTEFTLTISSLQPDDFATYYC

SEQ ID NO: 178 - protein, 36 aa, Homo sapiens
  REGION 1..36: note = ADT1-4 parental clones and ADT1-4-138 LFR3
SLESGVPSRFSGSGSGTEFTLTISSLQPDDFATYYC

SEQ ID NO: 179 - protein, 10 aa, Homo sapiens
  REGION 1..10: note = ADT1-4 LFR4 (parental and affinity matured clones)
FGQGTRLEIK

SEQ ID NO: 180 - protein, 10 aa, Homo sapiens
  REGION 1..10: note = ADT1-4-110 LFR4
FGAGTRLEIK

SEQ ID NO: 181 - protein, 10 aa, Homo sapiens
  REGION 1..10: note = ADT1-4-143 LFR4
FGQGTRLVIK

SEQ ID NO: 182 - protein, 10 aa, Homo sapiens
  REGION 1..10: note = ADT1-4-95 LFR4
FGSGTRLEIK

SEQ ID NO: 183 - protein, 9 aa, Homo sapiens
  REGION 1..9: note = ADT1-7-derived anti-TCR delta variable 1 antibody or antigen-binding fragment thereof: HCDR3
  VARIANT 1..2: note = X can be any naturally occurring amino acid
  VARIANT 4..5: note = X can be any naturally occurring amino acid
XXYXXAFDI

SEQ ID NO: 184 - protein, 9 aa, Homo sapiens
  REGION 1..9: note = ADT1-7-derived anti-TCR delta variable 1 antibody or antigen-binding fragment thereof: HCDR3
  VARIANT 1: note = X is D, I or V
  VARIANT 2: note = X is D or S
  VARIANT 4: note = X is N, E, D, Q or A
  VARIANT 5: note = X is D or E
XXYXXAFDI

SEQ ID NO: 185 - protein, 9 aa, Homo sapiens
  REGION 1..9: note = ADT1-7-derived anti-TCR delta variable 1 antibody or antigen-binding fragment thereof: HCDR3
  VARIANT 1: note = X is D or V
  VARIANT 2: note = X is D or S
  VARIANT 4: note = X is D, Q or A
  VARIANT 5: note = X is D or E
XXYXXAFDI

SEQ ID NO: 186 - protein, 9 aa, Homo sapiens
  REGION 1..9: note = ADT1-7-derived anti-TCR delta variable 1 antibody or antigen-binding fragment thereof: LCDR3
  VARIANT 3..6: note = X can be any naturally occurring amino acid
  VARIANT 8: note = X can be any naturally occurring amino acid
QQXXXXLXT

SEQ ID NO: 187 - protein, 9 aa, Homo sapiens
  REGION 1..9: note = ADT1-7-derived anti-TCR delta variable 1 antibody or antigen-binding fragment thereof: LCDR3
  VARIANT 3: note = X is T or S
  VARIANT 4: note = X is A, G, Y or S
  VARIANT 5: note = X is S or D
  VARIANT 6: note = X is T, E or G
  VARIANT 8: note = X is L or D
QQXXXXLXT

SEQ ID NO: 188 - protein, 9 aa, Homo sapiens
  REGION 1..9: note = ADT1-7-derived anti-TCR delta variable 1 antibody or antigen-binding fragment thereof: LCDR3
  VARIANT 3: note = X is S
  VARIANT 4: note = X is A or Y
  VARIANT 5: note = X is S
  VARIANT 6: note = X is E or G
  VARIANT 8: note = X is L or D
QQXXXXLXT

SEQ ID NO: 189 - protein, 25 aa, Homo sapiens
  REGION 1..25: note = ADT1-7 HFR1 (parental and all affinity matured clones)
EVQLVESGGGLVKPGGSLRLSCAAS

SEQ ID NO: 190 - protein, 17 aa, Homo sapiens
  REGION 1..17: note = ADT1-7 HFR2 (parental and all affinity matured clones)
MSWIRQAPGKGLEWVSY

SEQ ID NO: 191 - protein, 40 aa, Homo sapiens
  REGION 1..40: note = ADT1-7 HFR3 (parental and all affinity matured clones)
YYADSVKGRFTISRDNAKNSLYLQMNSLRAEDTAVYYCAR

SEQ ID NO: 192 - protein, 11 aa, Homo sapiens
  REGION 1..11: note = ADT1-7 HFR4 (parental and all affinity matured clones)
WGQGTLVTVSS

SEQ ID NO: 193 - protein, 26 aa, Homo sapiens
  REGION 1..26: note = ADT1-7 LFR1 (parental and affinity matured clones)
DIQMTQSPSSLSASVGDRVTIACRAG

SEQ ID NO: 194 - protein, 26 aa, Homo sapiens
  REGION 1..26: note = ADT1-7-18 LFR1
DIQMTQSPSSLSASVGDRVTIACRVG

SEQ ID NO: 195 - protein, 17 aa, Homo sapiens
  REGION 1..17: note = ADT1-7 LFR2 (parental and all affinity matured clones)
LNWYQQKPGKAPKLLIY

SEQ ID NO: 196 - protein, 36 aa, Homo sapiens
  REGION 1..36: note = ADT1-7 LFR3 (parental and all affinity matured clones)
SLQSGVPSRFSGSGSGTEFTLTISSLQPEDFATYYC

SEQ ID NO: 197 - protein, 10 aa, Homo sapiens
  REGION 1..10: note = ADT1-7 LFR4 (parental and all affinity matured clones)
FGRGTKVEIK

SEQ ID NO: 198 - DNA, 360 bp, Homo sapiens
  misc_feature 1..360: note = ADT1-4 Variable Heavy DNA
  source 1..360: mol_type = other DNA
caggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtggagtggttatgtggacgtctggggccaaggaaccctggtcaccgtctcgagc

SEQ ID NO: 199 - DNA, 360 bp, Homo sapiens
  misc_feature 1..360: note = ADT1-4-105 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggtgggttatgtggacgtctggggccaaggaaccctggtcaccgtctcgagc

SEQ ID NO: 200 - DNA, 360 bp, Homo sapiens
  misc_feature 1..360: note = ADT1-4-107 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggtgggttatgccgacgtctggggccaaggaaccctggtcaccgtctcgagc

SEQ ID NO: 201 - DNA, 360 bp, Homo sapiens
  misc_feature 1..360: note = ADT1-4-110 Variable Heavy DNA
  source 1..360: mol_type = other DNA
gaggtacagctgcagcagtcaggtccaggactggtgaagccctcgcagaccctctcactcacctgtgcgatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctctctgcagttaaactctgtgactcccgaggacacggctgtgtattactgtgcaagaacgtgggtggagtatgtggacgtctggggccaaggaaccctggtcaccgtctcgagc

SEQ ID NO: 202 - DNA, 360 bp, Homo sapiens
  misc_feature 1..360: note = ADT1-4-112 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggtgggttatgtggactactggggccaaggaaccctggtcaccgtctcgagc

SEQ ID NO: 203 - DNA, 360 bp, Homo sapiens; identical to SEQ ID NO: 199
  misc_feature 1..360: note = ADT1-4-117 Variable Heavy DNA
  source 1..360: mol_type = other DNA

SEQ ID NO: 204 - DNA, 360 bp, Homo sapiens
  misc_feature 1..360: note = ADT1-4-19 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggtgggttatgtggacaggtggggccaaggaaccctggtcaccgtctcgagc

SEQ ID NO: 205 - DNA, 360 bp, Homo sapiens
  misc_feature 1..360: note = ADT1-4-21 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgttgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggccgactatgtggacgtctggggccaaggaaccctggtcaccgtctcgagc

SEQ ID NO: 206 - DNA, 360 bp, Homo sapiens; identical to SEQ ID NO: 200
  misc_feature 1..360: note = ADT1-4-31 Variable Heavy DNA
  source 1..360: mol_type = other DNA

SEQ ID NO: 207 - DNA, 360 bp, Homo sapiens; identical to SEQ ID NO: 202
  misc_feature 1..360: note = ADT1-4-139 Variable Heavy DNA
  source 1..360: mol_type = other DNA

SEQ ID NO: 208 - DNA, 360 bp, Homo sapiens; identical to SEQ ID NO: 200
  misc_feature 1..360: note = ADT1-4-4 Variable Heavy DNA
  source 1..360: mol_type = other DNA

SEQ ID NO: 209 - DNA, 360 bp, Homo sapiens
  misc_feature 1..360: note = ADT1-4-143 Variable Heavy DNA
  source 1..360: mol_type = other DNA
gaggtacagctgctgcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggtgggttatgccgacgtctggggccaaggaaccctggtcaccgtctcgagc

SEQ ID NO: 210 - DNA, 360 bp, Homo sapiens; identical to SEQ ID NO: 205
  misc_feature 1..360: note = ADT1-4-53 Variable Heavy DNA
  source 1..360: mol_type = other DNA

SEQ ID NO: 211 - DNA, 360 bp, Homo sapiens
  misc_feature 1..360: note = ADT1-4-173 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggccggttatcccgacgtctggggccaaggaaccctggtcaccgtctcgagc

SEQ ID NO: 212 - DNA, 360 bp, Homo sapiens; identical to SEQ ID NO: 204
  misc_feature 1..360: note = ADT1-4-2 Variable Heavy DNA
  source 1..360: mol_type = other DNA

SEQ ID NO: 213 - DNA, 360 bp, Homo sapiens; identical to SEQ ID NO: 200
  misc_feature 1..360: note = ADT1-4-8 Variable Heavy DNA
  source 1..360: mol_type = other DNA

SEQ ID NO: 214 - DNA, 360 bp, Homo sapiens
  misc_feature 1..360: note = ADT1-4-82 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaagctgggtgggttatgtggacgtctggggccaaggaaccctggtcaccgtctcgagc

SEQ ID NO: 215 - DNA, 360 bp, Homo sapiens; identical to SEQ ID NO: 214
  misc_feature 1..360: note = ADT1-4-83 Variable Heavy DNA
  source 1..360: mol_type = other DNA

SEQ ID NO: 216 - DNA, 360 bp, Homo sapiens
  misc_feature 1..360: note = ADT1-4-3 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtgggccgactatgtggacgtctggggccaaggaaccctggtcaccgtctcgagc

SEQ ID NO: 217 - DNA, 360 bp, Homo sapiens; identical to SEQ ID NO: 200
  misc_feature 1..360: note = ADT1-4-84 Variable Heavy DNA
  source 1..360: mol_type = other DNA

SEQ ID NO: 218 - DNA, 360 bp, Homo sapiens
  misc_feature 1..360: note = ADT1-4-86 Variable Heavy DNA
  source 1..360: mol_type = other DNA
gaggtacagctgcagcagtcaggtccaggactggtgaagccctcgcagaccctctcactcacctgtgccatctccggggacagtgtctccagcaaaagtgttgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtggctgggtaacgtggacgtctggggccaaggaaccctggtcaccgtctcgagc

SEQ ID NO: 219 - DNA, 360 bp, Homo sapiens; identical to SEQ ID NO: 200
  misc_feature 1..360: note = ADT1-4-95 Variable Heavy DNA
  source 1..360: mol_type = other DNA

SEQ ID NO: 220 - DNA, 360 bp, Homo sapiens; identical to SEQ ID NO: 200
  misc_feature 1..360: note = ADT1-4-1 Variable Heavy DNA
  source 1..360: mol_type = other DNA

SEQ ID NO: 221 - DNA, 360 bp, Homo sapiens; identical to SEQ ID NO: 211
  misc_feature 1..360: note = ADT1-4-6 Variable Heavy DNA
  source 1..360: mol_type = other DNA

SEQ ID NO: 222 - DNA, 360 bp, Homo sapiens; identical to SEQ ID NO: 216
  misc_feature 1..360: note = ADT1-4-138 Variable Heavy DNA
  source 1..360: mol_type = other DNA

SEQ ID NO: 223 - DNA, 330 bp, Homo sapiens
  misc_feature 1..330: note = ADT1-4 Variable Light DNA
  source 1..330: mol_type = other DNA
gctagcgacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccatcaaggttcagcggcagtggatctgggacagaattcactctcaccatcagcagcctgcagcctgatgattttgcaacttactactgtcaacagagttacagtacccctcaggtcacttttggccaggggacacgactggagatcaaa

SEQ ID NO: 224 - DNA, 324 bp, Homo sapiens
  misc_feature 1..324: note = ADT1-4-105 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtacccctcagatcacttttggccaggggacacgactggagatcaaa

SEQ ID NO: 225 - DNA, 324 bp, Homo sapiens
  misc_feature 1..324: note = ADT1-4-107 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagcgctacagtacccctcagatcacttttggccaggggacacgactggagatcaaa

SEQ ID NO: 226 - DNA, 324 bp, Homo sapiens
  misc_feature 1..324: note = ADT1-4-110 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtacccctcaggtcacttttggcgccgggacacgactggagatcaaa

SEQ ID NO: 227 - DNA, 324 bp, Homo sapiens
  misc_feature 1..324: note = ADT1-4-112 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtcagcctcaggtcacttttggccaggggacacgactggagatcaaa

SEQ ID NO: 228 - DNA, 324 bp, Homo sapiens
  misc_feature 1..324: note = ADT1-4-117 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagcgctacagtacccctcgcgtcacttttggccaggggacacgactggagatcaaa

SEQ ID NO: 229 - DNA, 324 bp, Homo sapiens
  misc_feature 1..324: note = ADT1-4-19 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtacccctaaggtcacttttggccaggggacacgactggagatcaaa

SEQ ID NO: 230 - DNA, 324 bp, Homo sapiens
  misc_feature 1..324: note = ADT1-4-21 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtaccccttgggtcacttttggccaggggacacgactggagatcaaa

SEQ ID NO: 231 - DNA, 324 bp, Homo sapiens
  misc_feature 1..324: note = ADT1-4-31 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagcgctacagtacccctcccgtcacttttggccaggggacacgactggagatcaaa

SEQ ID NO: 232 - DNA, 324 bp, Homo sapiens
  misc_feature 1..324: note = ADT1-4-139 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagggctacagtacccctcagctgacttttggccaggggacacgactggagatcaaa

SEQ ID NO: 233 - DNA, 324 bp, Homo sapiens; identical to SEQ ID NO: 230
  misc_feature 1..324: note = ADT1-4-4 Variable Light DNA
  source 1..324: mol_type = other DNA

SEQ ID NO: 234 - DNA, 324 bp, Homo sapiens
  misc_feature 1..324: note = ADT1-4-143 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagcgctacagtacccaccaggtcacttttggccaggggacacgactggtgatcaaa

SEQ ID NO: 235 - DNA, 324 bp, Homo sapiens
  misc_feature 1..324: note = ADT1-4-53 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtacccctcagctgacttttggccaggggacacgactggagatcaaa

SEQ ID NO: 236 - DNA, 324 bp, Homo sapiens
  misc_feature 1..324: note = ADT1-4-173 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtgcccctcaggtcacttttggccaggggacacgactggagatcaaa

SEQ ID NO: 237 - DNA, 324 bp, Homo sapiens; identical to SEQ ID NO: 236
  misc_feature 1..324: note = ADT1-4-2 Variable Light DNA
  source 1..324: mol_type = other DNA

SEQ ID NO: 238 - DNA, 324 bp, Homo sapiens; identical to SEQ ID NO: 236
  misc_feature 1..324: note = ADT1-4-8 Variable Light DNA
  source 1..324: mol_type = other DNA

SEQ ID NO: 239 - DNA, 324 bp, Homo sapiens; identical to SEQ ID NO: 225
  misc_feature 1..324: note = ADT1-4-82 Variable Light DNA
  source 1..324: mol_type = other DNA

SEQ ID NO: 240 - DNA, 324 bp, Homo sapiens
  misc_feature 1..324: note = ADT1-4-83 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtgagcctcaggtcacttttggccaggggacacgactggagatcaaa

SEQ ID NO: 241 - DNA, 324 bp, Homo sapiens
  misc_feature 1..324: note = ADT1-4-3 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtacccctgaggtcacttttggccaggggacacgactggagatcaaa

SEQ ID NO: 242 - DNA, 324 bp, Homo sapiens
  misc_feature 1..324: note = ADT1-4-84 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtgaccctcaggtcacttttggccaggggacacgactggagatcaaa

SEQ ID NO: 243 - DNA, 324 bp, Homo sapiens; identical to SEQ ID NO: 240
  misc_feature 1..324: note = ADT1-4-86 Variable Light DNA
  source 1..324: mol_type = other DNA

SEQ ID NO: 244 - DNA, 324 bp, Homo sapiens
  misc_feature 1..324: note = ADT1-4-95 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacagtacccctcaggtcacttttggcagcgggacacgactggagatcaaa

SEQ ID NO: 245 - DNA, 324 bp, Homo sapiens
  misc_feature 1..324: note = ADT1-4-1 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagcgctacagtacccctatcgtcacttttggccaggggacacgactggagatcaaa

SEQ ID NO: 246 - DNA, 324 bp, Homo sapiens
  misc_feature 1..324: note = ADT1-4-6 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccattaaggttcagcggcagtggatctgggacagaattcactctcaccatcagcagcctgcagcctgatgattttgcaacttactactgtcaacagaagtacaagacccctcaggtcacttttggccaggggacacgactggagatcaaa

SEQ ID NO: 247 - DNA, 324 bp, Homo sapiens
  misc_feature 1..324: note = ADT1-4-138 Variable Light DNA
  source 1..324: mol_type = other DNA
gacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccatcaaggttcagcggcagtggatctgggacagaattcactctcaccatcagcagcctgcagcctgatgattttgcaacttactactgtcaacagcgctacagtaccgaccaggtcacttttggccaggggacacgactggagatcaaa

SEQ ID NO: 248 - DNA, 354 bp, Homo sapiens
  misc_feature 1..354: note = ADT1-7 Variable Heavy DNA
  source 1..354: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggtggactacgctgatgcatttgatatctggggccagggcaccctggtcaccgtctcgagc

SEQ ID NO: 249 - DNA, 354 bp, Homo sapiens
  misc_feature 1..354: note = ADT1-7-10 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggacgactacaacgatgcatttgatatctggggccagggcaccctggtcaccgtctcgagc

SEQ ID NO: 250 - DNA, 354 bp, Homo sapiens; identical to SEQ ID NO: 249
  misc_feature 1..354: note = ADT1-7-15 Variable Heavy DNA
  source 1..354: mol_type = other DNA

SEQ ID NO: 251 - DNA, 354 bp, Homo sapiens
  misc_feature 1..354: note = ADT1-7-17 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaaggatcgactacgaggatgcatttgatatctggggccagggcaccctggtcaccgtctcgagc

SEQ ID NO: 252 - DNA, 354 bp, Homo sapiens
  misc_feature 1..354: note = ADT1-7-18 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggtgagctacgacgatgcatttgatatctggggccagggcaccctggtcaccgtctcgagc

SEQ ID NO: 253 - DNA, 354 bp, Homo sapiens; identical to SEQ ID NO: 251
  misc_feature 1..354: note = ADT1-7-19 Variable Heavy DNA
  source 1..354: mol_type = other DNA

SEQ ID NO: 254 - DNA, 354 bp, Homo sapiens
  misc_feature 1..354: note = ADT1-7-20 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaattcactgtatctgcaaatgaacagcctgagagccgaggacacggctgtgtattactgtgcaagggtggactaccaggaggcatttgatatctggggccagggcaccctggtcaccgtctcgagc

SEQ ID NO: 255 - DNA, 354 bp, Homo sapiens; identical to SEQ ID NO: 249
  misc_feature 1..354: note = ADT1-7-22 Variable Heavy DNA
  source 1..354: mol_type = other DNA

SEQ ID NO: 256 - DNA, 354 bp, Homo sapiens
  misc_feature 1..354: note = ADT1-7-23 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggtggactacaacgaggcatttgatatctggggccagggcaccctggtcaccgtctcgagc

SEQ ID NO: 257 - DNA, 354 bp, Homo sapiens
  misc_feature 1..354: note = ADT1-7-42 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggacgactacgaggatgcatttgatatctggggccagggcaccctggtcaccgtctcgagc

SEQ ID NO: 258 - DNA, 354 bp, Homo sapiens
  misc_feature 1..354: note = ADT1-7-3 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggtgagctacgctgaggcatttgatatctggggccagggcaccctggtcaccgtctcgagc

SEQ ID NO: 259 - DNA, 354 bp, Homo sapiens
  misc_feature 1..354: note = ADT1-7-61 Variable Heavy DNA
  source 1..354: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggacgactacgacgatgcatttgatatctggggccagggcaccctggtcaccgtctcgagc

SEQ ID NO: 260 - DNA, 327 bp, Homo sapiens
  misc_feature 1..327: note = ADT1-7 Variable Light DNA
  source 1..327: mol_type = other DNA
gctagcgacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagttacagtaccctcctcactttcggcagagggaccaaggtggaaatcaaa

SEQ ID NO: 261 - DNA, 321 bp, Homo sapiens
  misc_feature 1..321: note = ADT1-7-10 Variable Light DNA
  source 1..321: mol_type = other DNA
gacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagaccgccagtaccctcctcactttcggcagagggaccaaggtggaaatcaaa

SEQ ID NO: 262 - DNA, 321 bp, Homo sapiens
  misc_feature 1..321: note = ADT1-7-15 Variable Light DNA
  source 1..321: mol_type = other DNA
gacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagtgccgacaccctcctcactttcggcagagggaccaaggtggaaatcaaa

SEQ ID NO: 263 - DNA, 321 bp, Homo sapiens
  misc_feature 1..321: note = ADT1-7-17 Variable Light DNA
  source 1..321: mol_type = other DNA
gacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagtggcagtgagctcctcactttcggcagagggaccaaggtggaaatcaaa

SEQ ID NO: 264 - DNA, 321 bp, Homo sapiens
  misc_feature 1..321: note = ADT1-7-18 Variable Light DNA
  source 1..321: mol_type = other DNA
gacatccagatgacccagtctccatcctccctgtctgcatctgtaggagacagagtcaccatcgcttgccgggtaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagtgccagtgagctcctcactttcggcagagggaccaaggtggaaatcaaa

SEQ ID NO: 265 - DNA, 321 bp, Homo sapiens
  misc_feature 1..321: note = ADT1-7-19 Variable Light DNA
  source 1..321: mol_type = other DNA
gacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagtgccagtgagctcctcactttcggcagagggaccaaggtggaaatcaaa

SEQ ID NO: 266 - DNA, 321 bp, Homo sapiens
  misc_feature 1..321: note = ADT1-7-20 Variable Light DNA
  source 1..321: mol_type = other DNA
gacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagttacagtggcctcgacactttcggcagagggaccaaggtggaaatcaaa

SEQ ID NO: 267 - DNA, 321 bp, Homo sapiens; identical to SEQ ID NO: 265
  misc_feature 1..321: note = ADT1-7-22 Variable Light DNA
  source 1..321: mol_type = other DNA

SEQ ID NO: 268 - DNA, 321 bp, Homo sapiens; identical to SEQ ID NO: 266
  misc_feature 1..321: note = ADT1-7-23 Variable Light DNA
  source 1..321: mol_type = other DNA

SEQ ID NO: 269 - DNA, 321 bp, Homo sapiens
  misc_feature 1..321: note = ADT1-7-42 Variable Light DNA
  source 1..321: mol_type = other DNA
gacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagtagcagtgagctcctcactttcggcagagggaccaaggtggaaatcaaa

SEQ ID NO: 270 - DNA, 321 bp, Homo sapiens; identical to SEQ ID NO: 266
  misc_feature 1..321: note = ADT1-7-3 Variable Light DNA
  source 1..321: mol_type = other DNA

SEQ ID NO: 271 - DNA, 321 bp, Homo sapiens; identical to SEQ ID NO: 265
  misc_feature 1..321: note = ADT1-7-61 Variable Light DNA
  source 1..321: mol_type = other DNA

SEQ ID NO: 272 - protein, 209 aa, Homo sapiens
  REGION 1..209: note = Human variable delta 1 (V delta 1) chain of a gamma delta T cell receptor (TCR) - aka TRDV1
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGESLTRADKLIFGKGTRVTVEPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 273 - protein, 117 aa, Homo sapiens
  REGION 1..117: note = C08 Variable Heavy
EVQLLESGGGLVQPGRSLRLSCAASGFTVSSNYMSWVRQAPGKGLEWVSVIYSGGSTYYADSVKGRFTISRDNSKNTLYLQMNSLRAEDTAVYYCASPIELGAFDIWGQGTLVTVSS

SEQ ID NO: 274 - protein, 118 aa, Homo sapiens
  REGION 1..118: note = B07 Variable Heavy
EVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARENYLNAFDIWGRGTLVTVSS

SEQ ID NO: 275 - protein, 117 aa, Homo sapiens
  REGION 1..117: note = C05 Variable Heavy
EVQLLESGGGLVQPGGSLRLSCAASGFTFSSYAMSWVRQAPGKGLEWVSAISGGGGTTYSSDSVKGRFTISRDNSKNTLYLQMNSLRAEDTAVYYCARDSGVAFDIWGQGTLVTVSS

SEQ ID NO: 276 - protein, 120 aa, Homo sapiens
  REGION 1..120: note = E04 Variable Heavy
QVQLQQSGPGLVKPSQTLSLTCAISGDSVSSNSAAWNWIRQSPSRGLEWLGRTYYRSKWYNDYAVSVRSRITINPDTSKNQFSLQLNSVTPEDTAVYYCARSWNDAFDIWGQGTTVTVSS

SEQ ID NO: 277 - protein, 120 aa, Homo sapiens
  REGION 1..120: note = F07 Variable Heavy
QVQLQQSGPGLVKPSQTLSLTCVISGDSVSSNSAAWNWIRQSPSRGLEWLGRTYYRSKWYNDYAVSVKSRITINPDTSKNQFSLQLNSVTPEDTAVYYCARDYYYSMDVWGQGTMVTVSS

SEQ ID NO: 278 - protein, 118 aa, Homo sapiens
  REGION 1..118: note = G06 Variable Heavy
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWNDAFDVWGQGTLVTVSS

SEQ ID NO: 279 - protein, 120 aa, Homo sapiens
  REGION 1..120: note = G09 Variable Heavy
QVQLQQSGPGLVKPSQTLSLTCAISGDSVSSNSAAWNWIRQSPSRGLEWLGRTYYGSKWYNEYALSVKSRIIINPDTSKNQFSLQLNSVTPEDTAVYYCARDYYYSMDVWGQGTLVTVSS

SEQ ID NO: 280 - protein, 118 aa, Homo sapiens
  REGION 1..118: note = B09 Variable Heavy
EVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWSDAFDIWGQGTLVTVSS

SEQ ID NO: 281 - protein, 118 aa, Homo sapiens
  REGION 1..118: note = G10 Variable Heavy
EVQLVESGGGLVQPGGSLRLSCAASGFTFSDYYMSWIRQAPGKGLEWVSYISSSGSTIYYADSVKGRFTISRDNAKNSLYLQMNSLRAEDTAVYYCARHSWNDAFDIWGRGTLVTVSS

SEQ ID NO: 282 - protein, 110 aa, Homo sapiens
  REGION 1..110: note = C08 Variable Light
ASSYELTQPPSVSVAPGKTARITCGGNNIGSQSVHWYQQKPGQAPMLVIYYDSDRPSGIPERFSGSNSGNTATLTISRVEAGDEADYYCQVWDSSSDHVVFGGGTKLTVL

SEQ ID NO: 283 - protein, 109 aa, Homo sapiens
  REGION 1..109: note = B07 Variable Light
ASDIQMTQSPSSLSASVGDRVTITCRTSQSLSNYLNWYQQKPGKAPKLLIYAASSLQSGVPSRFSGSGSGTDFTLTISSLQPEDFATYYCQQSYSTPLTFGGGTKLEIK

SEQ ID NO: 284 - protein, 109 aa, Homo sapiens
  REGION 1..109: note = C05 Variable Light
ASDIQMTQSPSFLSASVGDRVTITCRASQNIRTWLAWYQQKPGRAPKLLIYDASSLESGVPSRFSGSGSGTDFTLTISSLQPEDFATYYCQQFKRYPPTFGLGTKVEIK

SEQ ID NO: 285 - protein, 109 aa, Homo sapiens
  REGION 1..109: note = E04 Variable Light
ASDIVMTQSPSTLSASIGDRVTITCRAGQSISTWLAWYQQKPGKAPKLLIYDASSLESGVPLRFSGSGSGTDFTLTISSLQPEDFATYYCQQSYSTPLTFGGGTKVEIK

SEQ ID NO: 286 - protein, 109 aa, Homo sapiens
  REGION 1..109: note = F07 Variable Light
ASDIQMTQSPSTLSASVGDRVTITCRASQSISSWLAWYQQKPGKAPKLLIYDASSLESGVPLRFSGSGSGTEFTLTISSLQPDDFATYYCQQSHSHPPTFGPGTKVDIK

SEQ ID NO: 287 - protein, 109 aa, Homo sapiens
  REGION 1..109: note = G06 Variable Light
ASDIQMTQSPSSLSASVGDRVSITCRASQSISSYLNWYQQKPGKAPKLLIYAASSLQSGVPSRFSGSGSGTDFTLTISSLQPEDFATYYCQQSYSTPDTFGGGTKVEIK

SEQ ID NO: 288 - protein, 109 aa, Homo sapiens
  REGION 1..109: note = G09 Variable Light
ASDIVMTQSPSTLSASIGDRVTITCRAGQSISTWLAWYQQKPGKAPKLLIYDASSLESGVPLRFSGSGSGTEFTLAISSLQPDDFATYYCQQSYSTPVTFGQGTKVEIK

SEQ ID NO: 289 - protein, 109 aa, Homo sapiens
  REGION 1..109: note = B09 Variable Light
ASDIQMTQSPSSLSASVGDRVTITCQASQDISNYLNWYQQKPGKAPKLLIYDASNLETGVPSRFSGSGSGTDFTLTISSLQPEDFATYYCQQSYSTPLTFGGGTKVEIK

SEQ ID NO: 290 - protein, 109 aa, Homo sapiens
  REGION 1..109: note = G10 Variable Light
ASDIQMTQSPSSLSASVGDRVTITCRASQSISSHLNWYQQKPGKAPKLLIYAASSLQSGVPSRFSASGSGTDFTLTISSLQPEDFATYYCQQSYSTLLTFGGGTKVEIK

SEQ ID NO: 291 - protein, 13 aa, synthetic construct
  REGION 1..13: note = VH-VL linker
GGGGSGGGGSGGG

SEQ ID NO: 292 - protein, 12 aa, synthetic construct
  REGION 1..12: note = Optional scFv tag sequence (kappa sequence + His tag)
RTAAASAHHHHH

SEQ ID NO: 293 - protein, 37 aa, synthetic construct
  REGION 1..37: note = Optional scFv tag sequence (kappa sequence + His and FLAG tag)
RTAAASAHHHHHHKLDYKDHDGDYKDHDIDYKDDDDK

SEQ ID NO: 294 - protein, 13 aa, synthetic construct
  REGION 1..13: note = Optional scFv tag sequence (lambda sequence + His tag)
GQPAAASAHHHHH

SEQ ID NO: 295 - protein, 38 aa, synthetic construct
  REGION 1..38: note = Optional scFv tag sequence (lambda sequence + His and FLAG tag)
GQPAAASAHHHHHHKLDYKDHDGDYKDHDIDYKDDDDK

SEQ ID NO: 296 - protein, 107 aa, Homo sapiens [sequence omitted]

SEQ ID NO: 297 - protein, 330 aa, Homo sapiens [sequence omitted]

SEQ ID NO: 298 - protein, 330 aa, Homo sapiens
  REGION 1..330: note = Constant domain (AD4 full heavy constant domain chain employed (with L235A, G237A modification))
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 20 aa, Homo sapiens
  REGION 1..20: note = N-terminal leader sequence of the V delta 1 chain
MLFSSLLCVFVAFSYSGSSV

SEQ ID NO: 300 - protein, 95 aa, Homo sapiens
  REGION 1..95: note = V region of the V delta 1 chain
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGE

SEQ ID NO: 301 - protein, 16 aa, Homo sapiens
  REGION 1..16: note = J1*0 - one of the four J regions encoded in the human delta one chain germline
TDKLIFGKGTRVTVEP

SEQ ID NO: 302 - protein, 17 aa, Homo sapiens
  REGION 1..17: note = J2*0 - one of the four J regions encoded in the human delta one chain germline
LTAQLFFGKGTQLIVEP

SEQ ID NO: 303 - protein, 19 aa, Homo sapiens
  REGION 1..19: note = J3*0 - one of the four J regions encoded in the human delta one chain germline
SWDTRQMFFGTGIKLFVEP

SEQ ID NO: 304 - protein, 15 aa, Homo sapiens
  REGION 1..15: note = J4*0 - one of the four J regions encoded in the human delta one chain germline
RPLIFGKGTYLEVQQ

SEQ ID NO: 305 - protein, 153 aa, Homo sapiens
  REGION 1..153: note = C1*0 which contains the C-terminal juxtamembrane/transmembrane regions
  VARIANT 1: note = X can be any naturally occurring amino acid
XSQPHTKPSVFVMKNGTNVACLVKEFYPKDIRINLVSSKKITEFDPAIVISPSGKYNAVKLGKYESNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 306 - protein, 254 aa, Homo sapiens
  REGION 1..254: note = Human variable delta 1 (V delta 1) chain of a gamma delta T cell receptor (TCR) polymorphic variant - aka human TRDV1 polymorphic variant
AQKVTQAQSSVSMPVRKAVTLNCLYETSWWSYYIFWYKQLPSKEMIFLIRQGSDEQNAKSGRYSVNFKKAVKSVALTISALQLEDSAKYFCALGESLTRADKLIFGKGTRVTVEPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCTTAPSAQLKKKLQALKKKNAQLKWKLQALKKKLAQGSGHHHHHH

SEQ ID NO: 307 - protein, 107 aa, Homo sapiens
  REGION 1..107: note = Constant domain (AD3 and AD4 full light constant chain with RTAAAPS sequence)
RTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 92 aa, Homo sapiens
  REGION 1..92: note = Cyno variable delta 1 (V delta 1) chain of a gamma delta T cell receptor (TCR) - aka cyno TRDV1
AQKVTQAQSSVSMPVEKAVTLNCQYETSSWSYDLFWYKQLPGKEMIFLIHQGSSQQNARNGRYSVNFQKAASSITLTISALQLEDSATYFCA

SEQ ID NO: 309 - protein, 101 aa, Homo sapiens
  REGION 1..101: note = Human variable gamma 4 chain (V gamma 4) of a gamma delta T cell receptor (TCR) - aka human TRGV4
KSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDG

SEQ ID NO: 310 - protein, 96 aa, Homo sapiens
  REGION 1..96: note = Human variable delta 2 (V delta 2) chain of a gamma delta T cell receptor (TCR) - aka TRDV2
AIELVPEHQTVPVSIGVPATLRCSMKGEAIGNYYINWYRKTQGNTMTFIYREKDIYGPGFKDNFQGDIDIAKNLAVLKILAPSERDEGSYYCACDT

SEQ ID NO: 311 - protein, 95 aa, Homo sapiens
  REGION 1..95: note = Human variable delta 3 (V delta 3) chain of a gamma delta T cell receptor (TCR) - aka TRDV3
DKVTQSSPDQTVASGSEVVLLCTYDTVYSNPDLFWYRIRPDYSFQFVFYGDNSRSEGADFTQGRFSVKHILTQKAFHLVISPVRTEDSATYYCAF

SEQ ID NO: 312 - protein, 119 aa, Homo sapiens
  REGION 1..119: note = E01 Variable Heavy
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHQVDTRTADYWGQGTLVTVSS

SEQ ID NO: 313 - protein, 112 aa, Homo sapiens
  REGION 1..112: note = E01 Variable Light
ASQSALTQPASVSGSPGQSVTISCTGTRSDVGGYNYVSWYQHHPGKAPKLMIYEVSNRPSGVSNRFSGSKSGNTASLTISGLQAEDEADYYCSSYTSTSTLVFGGGTKLTVL

SEQ ID NO: 314-316 - skipped (3 entries)

SEQ ID NO: 317 - protein, 8 aa, Homo sapiens
  REGION 1..8: note = C08 HCDR1
GFTVSSNY

SEQ ID NO: 318 - protein, 7 aa, Homo sapiens
  REGION 1..7: note = C08 HCDR2
IYSGGST

SEQ ID NO: 319 - protein, 9 aa, Homo sapiens
  REGION 1..9: note = C08 HCDR3
PIELGAFDI

SEQ ID NO: 320 - protein, 8 aa, Homo sapiens
  REGION 1..8: note = B07 HCDR1
GFTFSDYY

SEQ ID NO: 321 - protein, 8 aa, Homo sapiens
  REGION 1..8: note = B07 HCDR2
ISSSGSTI

SEQ ID NO: 322 - protein, 9 aa, Homo sapiens
  REGION 1..9: note = B07 HCDR3
ENYLNAFDI

SEQ ID NO: 323 - protein, 8 aa, Homo sapiens
  REGION 1..8: note = C05 HCDR1
GFTFSSYA

SEQ ID NO: 324 - protein, 8 aa, Homo sapiens
  REGION 1..8: note = C05 HCDR2
ISGGGGTT

SEQ ID NO: 325 - protein, 8 aa, Homo sapiens
  REGION 1..8: note = C05 HCDR3
DSGVAFDI

SEQ ID NO: 326 - protein, 10 aa, Homo sapiens
  REGION 1..10: note = E04 HCDR1
GDSVSSNSAA

SEQ ID NO: 327 - protein, 9 aa, Homo sapiens
  REGION 1..9: note = E04 HCDR2
TYYRSKWYN

SEQ ID NO: 328 - protein, 8 aa, Homo sapiens
  REGION 1..8: note = E04 HCDR3
SWNDAFDI

SEQ ID NO: 329 - protein, 10 aa, Homo sapiens
  REGION 1..10: note = F07 HCDR1
GDSVSSNSAA

SEQ ID NO: 330 - protein, 9 aa, Homo sapiens
  REGION 1..9: note = F07 HCDR2
TYYRSKWYN

SEQ ID NO: 331 - protein, 8 aa, Homo sapiens
  REGION 1..8: note = F07 HCDR3
DYYYSMDV

SEQ ID NO: 332 - protein, 8 aa, Homo sapiens
  REGION 1..8: note = G06 HCDR1
GFTFSDYY

SEQ ID NO: 333 - protein, 8 aa, Homo sapiens
  REGION 1..8: note = G06 HCDR2
ISSSGSTI

SEQ ID NO: 334 - protein, 9 aa, Homo sapiens
  REGION 1..9: note = G06 HCDR3
HSWNDAFDV

SEQ ID NO: 335 - protein, 10 aa, Homo sapiens
  REGION 1..10: note = G09 HCDR1
GDSVSSNSAA

SEQ ID NO: 336 - protein, 9 aa, Homo sapiens
  REGION 1..9: note = G09 HCDR2
TYYGSKWYN

SEQ ID NO: 337 - skipped

SEQ ID NO: 338 - protein, 8 aa, Homo sapiens
  REGION 1..8: note = B09 HCDR1
GFTFSDYY

SEQ ID NO: 339 - protein, 8 aa, Homo sapiens
  REGION 1..8: note = B09 HCDR2
ISSSGSTI

SEQ ID NO: 340 - protein, 9 aa, Homo sapiens
  REGION 1..9: note = B09 HCDR3
HSWSDAFDI

SEQ ID NO: 341 - protein, 8 aa, Homo sapiens
  REGION 1..8: note = G10 HCDR1
GFTFSDYY

SEQ ID NO: 342 - protein, 8 aa, Homo sapiens
  REGION 1..8: note = G10 HCDR2
ISSSGSTI

SEQ ID NO: 343 - protein, 9 aa, Homo sapiens
  REGION 1..9: note = G10 HCDR3
HSWNDAFDI

SEQ ID NO: 344 - protein, 8 aa, Homo sapiens
  REGION 1..8: note = E01 HCDR1
GYSFTSYW

SEQ ID NO: 345 - protein, 8 aa, Homo sapiens
  REGION 1..8: note = E02 HCDR2
IYPGDSDT

SEQ ID NO: 346 - protein, 10 aa, Homo sapiens
  REGION 1..10: note = E02 HCDR3
HQVDTRTADY

SEQ ID NO: 347 - protein, 6 aa, Homo sapiens
  REGION 1..6: note = C08 LCDR1
NIGSQS

SEQ ID NO: 348 - skipped

SEQ ID NO: 349 - protein, 11 aa, Homo sapiens
  REGION 1..11: note = C08 LCDR3
QVWDSSSDHVV

SEQ ID NO: 350 - protein, 6 aa, Homo sapiens
  REGION 1..6: note = B07 LCDR1
QSLSNY

SEQ ID NO: 351 - skipped

SEQ ID NO: 352 - protein, 9 aa, Homo sapiens
  REGION 1..9: note = B07 LCDR3
QQSYSTPLT

SEQ ID NO: 353 - protein, 6 aa, Homo sapiens
  REGION 1..6: note = C05 LCDR1
QNIRTW

SEQ ID NO: 354 - skipped

SEQ ID NO: 355 - protein, 9 aa, Homo sapiens
  REGION 1..9: note = C05 LCDR3
QQFKRYPPT

SEQ ID NO: 356 - protein, 6 aa, Homo sapiens
  REGION 1..6: note = E04 LCDR1
QSISTW

SEQ ID NO: 357 - skipped

SEQ ID NO: 358 - protein, 9 aa, Homo sapiens
  REGION 1..9: note = E04 LCDR3
QQSYSTPLT

SEQ ID NO: 359 - protein, 6 aa, Homo sapiens
  REGION 1..6: note = F07 LCDR1
QSISSW

SEQ ID NO: 360 - skipped

SEQ ID NO: 361 - protein, 9 aa, Homo sapiens
  REGION 1..9: note = F07 LCDR3
QQSHSHPPT

SEQ ID NO: 362 - protein, 6 aa, Homo sapiens
  REGION 1..6: note = G06 LCDR1
QSISSY

SEQ ID NO: 363 - skipped

SEQ ID NO: 364 - protein, 9 aa, Homo sapiens
  REGION 1..9: note = G06 LCDR3
QQSYSTPDT

SEQ ID NO: 365 - protein, 6 aa, Homo sapiens
  REGION 1..6: note = G09 LCDR1
QSISTW

SEQ ID NO: 366 - skipped

SEQ ID NO: 367 - protein, 9 aa, Homo sapiens
  REGION 1..9: note = G09 LCDR3
QQSYSTPVT

SEQ ID NO: 368 - protein, 6 aa, Homo sapiens
  REGION 1..6: note = B09 LCDR1
QDISNY

SEQ ID NO: 369 - skipped

SEQ ID NO: 370 - protein, 9 aa, Homo sapiens
  REGION 1..9: note = B09 LCDR3
QQSYSTPLT

SEQ ID NO: 371 - protein, 6 aa, Homo sapiens
  REGION 1..6: note = G10 LCDR1
QSISSH

SEQ ID NO: 372 - skipped

SEQ ID NO: 373 - protein, 9 aa, Homo sapiens
  REGION 1..9: note = G10 LCDR3
QQSYSTLLT

SEQ ID NO: 374 - protein, 9 aa, Homo sapiens
  REGION 1..9: note = E01 LCDR1
RSDVGGYNY

SEQ ID NO: 375 - skipped

SEQ ID NO: 376 - protein, 10 aa, Homo sapiens
  REGION 1..10: note = E02 LCDR3
SSYTSTSTLV

SEQ ID NO: 377-413 - skipped (37 entries)

SEQ ID NO: 414 - protein, 215 aa, Homo sapiens
  REGION 1..215: note = ADT1-4-2 LC
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 415-420 - skipped (6 entries)

SEQ ID NO: 421 - protein, 217 aa, Homo sapiens
  REGION 1..217: note = G04 LC
ASDIQMTQSPPALSASVGDRVTITCRASQDINDWLAWYQHKPGKAPKLLIYDASSLESGVPSRFSGSGSGTEFTLTISSLQPDDFATYYCQQSYSTPQVTFGQGTRLEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 422 - skipped

SEQ ID NO: 423 - protein, 216 aa, Homo sapiens
  REGION 1..216: note = E07 LC
ASDIQMTQSPSSLSASVGDRVTIACRAGQSIGTYLNWYQQKPGKAPKLLIYVASSLQSGVPSRFSGSGSGTEFTLTISSLQPEDFATYYCQQSYSTLLTFGRGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 424 - skipped

SEQ ID NO: 425 - protein, 216 aa, Homo sapiens
  REGION 1..216: note = C08 LC
ASSYELTQPPSVSVAPGKTARITCGGNNIGSQSVHWYQQKPGQAPMLVIYYDSDRPSGIPERFSGSNSGNTATLTISRVEAGDEADYYCQVWDSSSDHVVFGGGTKLTVLGQPAAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 426-563 - skipped (138 entries)

SEQ ID NO: 564 - protein, 106 aa, Homo sapiens
GQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 565 - protein, 4 aa, Homo sapiens
TGYW

SEQ ID NO: 566 - protein, 6 aa, Homo sapiens
EPQYWA

SEQ ID NO: 567 - protein, 9 aa, Homo sapiens
SNWRWQLGD

SEQ ID NO: 568 - protein, 106 aa, Homo sapiens
GQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 569 - protein, 4 aa, Homo sapiens
EGYW

SEQ ID NO: 570 - protein, 105 aa, Homo sapiens
GQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 571 - protein, 4 aa, Homo sapiens
SGYW

SEQ ID NO: 572 - protein, 9 aa, Homo sapiens
SNWRWQLDD

SEQ ID NO: 573 - protein, 106 aa, Homo sapiens
GQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 574 - protein, 106 aa, Homo sapiens
GQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 575 - protein, 9 aa, Homo sapiens
SKWRWQLDD

SEQ ID NO: 576 - protein, 106 aa, Homo sapiens
GQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 577 - protein, 106 aa, Homo sapiens
GQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 578 - protein, 4 aa, Homo sapiens
HGYW

SEQ ID NO: 579 - protein, 106 aa, Homo sapiens
GQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 580 - protein, 9 aa, Homo sapiens
SHWRWQLDD

SEQ ID NO: 581 - protein, 106 aa, Homo sapiens
GQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 582 - protein, 9 aa, Homo sapiens
SNWRWRLDD

SEQ ID NO: 583 - protein, 106 aa, Homo sapiens
GQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 584 - protein, 9 aa, Homo sapiens
SNWRWSLDD

SEQ ID NO: 585 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 586 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 587 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 588 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 589 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 590 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 591 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 592 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 593 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 594 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLGDVFSCSVMHEALRNHYTQKSLSLSPGK

SEQ ID NO: 595 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 596 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 597 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 598 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 599 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 600 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 601 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 602 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 603 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 604 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLGDVFSCSVMHEALHNHYTQKSLSLSPGK

SEQ ID NO: 605 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 606 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 607 - protein, 448 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 608 - protein, 448 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 609 - protein, 663 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 610 - protein, 663 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 611 - protein, 448 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 612 - protein, 448 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 613 - protein, 663 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 614 - protein, 663 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWQLDDFSCSVMHEALHNHYTQKSLSLSPGK

SEQ ID NO: 615 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 616 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 617 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 618 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 619 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 620 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 621 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 622 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 623 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 624 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 625 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 626 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 627 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 628 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 629 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 630 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 631 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 632 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 633 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 634 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETGYWVSLTCLVKGFYPSDIAVEWESNGEPQYWAKTTPPVLDSDGSFFLYSKLTVSKWRWQLDDVFSCSVMHEALHNHYTQKSLSLSPGK

SEQ ID NO: 635 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 636 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 637 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 638 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 639 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 640 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 641 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 642 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 643 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 644 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEEGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 645 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 646 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 647 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 648 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 649 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 650 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 651 - protein, 449 aa, Homo sapiens; identical to SEQ ID NO: 650

SEQ ID NO: 652 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 653 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 654 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEHGYWVSLTCLVKGFYPSDIAVEWESNGEPQYWAKTTPPVLDSDGSFFLYSKLTVSNWRWQLDDVFSCSVMHEALHNHYTQKSLSLSPGK

SEQ ID NO: 655 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 656 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 657 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 658 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 659 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 660 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 661 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 662 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 663 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 664 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSHWRWQLDDVFSCSVMHEALHNHYTQKSLSLSPGK

SEQ ID NO: 665 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 666 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 667 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 668 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 669 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 670 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 671 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 672 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 673 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 674 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWRLDDVFSCSVMHEALHNHYTQKSLSLSPGK

SEQ ID NO: 675 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 676 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 677 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 678 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 679 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 680 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQKYSAPQVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYVDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 681 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 682 - protein, 449 aa, Homo sapiens
EVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 683 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 684 - protein, 664 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWVGYADVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDESGYWVSLTCLVKGFYPSDIAVEWESNGEPQYWAKTTPPVLDSDGSFFLYSKLTVSNWRWSLDDVFSCSVMHEALHNHYTQKSLSLSPGK

SEQ ID NO: 685 - protein, 215 aa, Homo sapiens
DIQMTQSPPALSASVGDRVTITCRASQDINDWLAWYQHKPGKAPKLLIYDASSLESGVPLRFSGSGSGTEFTLTISSLQPDDFATYYCQQRYSTPIVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 686 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 687 - protein, 40 aa, synthetic construct
  VARIANT 6..40: note = may be absent
GGGGSGGGGSGGGGSGGGGSGGGGSGGGGSGGGGSGGGGS

SEQ ID NO: 688 - protein, 640 aa, synthetic construct
  VARIANT 6..40: note = may be absent
  VARIANT 46..80: note = may be absent
  VARIANT 81..640: note = may be absent
GGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGASGGGGSGGGGSGGGGSGGGGSGGGGSGGGGSGGGGSGGGGSGGGASGGGASGGGASGGGASGGGASGGGASGGGASGGGAS

SEQ ID NO: 689 - protein, 7 aa, synthetic construct
RTAAAPS

SEQ ID NO: 690 - protein, 7 aa, synthetic construct
RTVAAPS

SEQ ID NO: 691 - protein, 8 aa, synthetic construct
GQPAAAPS

SEQ ID NO: 692 - protein, 8 aa, synthetic construct
GQPKAAPS

SEQ ID NO: 693 - protein, 4 aa, synthetic construct
GQPK

(19) United States
(12) Patent Application Publication
(10) Pub. No.: US 2026/0152558 A1
(43) Pub. Date: Jun. 4, 2026

(54) MULTISPECIFIC ANTI-TCR DELTA VARIABLE 1 ANTIBODIES

(71) Applicants: GammaDelta Therapeutics Ltd, London (GB); F-star Therapeutics Limited, Cambridge (GB)

(72) Inventors: Natalie Mount, London (GB); Mihriban Tuna, Cambridge (GB); Mark Uden, London (GB); Robert Good, London (GB); Shauna Corcoran, London (GB); Andrew Hutton, Boston, MA (US); Antara Banerjee, San Diego, CA (US); Kathy Seidel, London (GB); Patrick LeRoy, Boston, MA (US); Taylor Hickman, Cambridge, MA (US)

(73) Assignees: GammaDelta Therapeutics Ltd, London (GB); F-star Therapeutics Limited, Cambridge (GB)

(21) Appl. No.: 19/402,538

(22) Filed: Nov. 26, 2025

Related U.S. Application Data
(60) Provisional application No. 63/726,148, filed on Nov. 27, 2024.
(60) Provisional application No. 63/814,080, filed on May 29, 2025.

(51) Int. Cl. C07K 16/28; A61K 39/00; A61P 35/00

(52) U.S. Cl. CPC C07K 16/2809; A61P 35/00; C07K 16/2827; A61K 2039/505; C07K 2317/31; C07K 2317/526; C07K 2317/55; C07K 2317/565; C07K 2317/92

(57) ABSTRACT

This disclosure includes multispecific antibodies comprising a Fab region and an Fc region, wherein the Fab region comprises a binding site specific for an epitope of the variable delta 1 (Vδ1) chain of a γδ T cell receptor (TCR); and the Fc region comprises an PD-L1 binding site. This disclosure also includes compositions and pharmaceutical compositions comprising such multispecific antibodies, and methods of making such multispecific antibodies. The present disclosure also includes methods of treatment and medical uses involving the multispecific antibodies.

### CROSS-REFERENCE TO RELATED APPLICATIONS

This application claims the benefit under 35 U.S.C. § 119(e) of U.S. provisional application No. 63/726,148, filed Nov. 27, 2024 and U.S. provisional application No. 63/814,080, filed May 29, 2025, the entire contents of each of which are incorporated herein by reference.

### REFERENCE TO AN ELECTRONIC SEQUENCE LISTING

The contents of the electronic sequence listing (T083370057US02-SUBSEQ-ARM.xml; Size: 712,996 bytes; and Date of Creation: Dec. 23, 2025) are herein incorporated by reference in their entirety.

### FIELD

The present disclosure relates to multispecific antibodies and fragments and variants thereof that specifically bind the T cell receptor of gamma delta T cells and specifically bind to PD-L1.

### BACKGROUND

The growing interest in T cell immunotherapy for cancer has largely focused on the evident capacity of subsets of CD8+ and CD4+ alpha beta (αβ) T cells to recognize cancer cells and to mediate host-protective functional potentials. However, αβ T cells are MHC-restricted which can lead to graft versus host disease.

Gamma delta T cells (γδ T cells) represent a subset of T cells that express on their surface a distinct, defining γδ T-cell receptor (TCR). This TCR is made up of one gamma (γ) and one delta (δ) chain, each of which undergoes chain rearrangement but have a limited number of V genes as compared to αβ T cells. The main TRGV gene segments encoding Vγ are TRGV2, TRGV3, TRGV4, TRGV5, TRGV8, TRGV9 and TRGV11 and non-functional genes TRGV10, TRGV11, TRGVA and TRGVB. The most frequent TRDV gene segments encode Vδ1, Vδ2, and Vδ3, plus several V segments that have both Vδ and Vα designation (Adams et al., 296:30-40 (2015) Cell Immunol.). Human γδ T cells can be broadly classified based on their TCR chains, as certain γ and δ types are found on cells more prevalently, though not exclusively, in one or more tissue types. For example, most blood-resident γδ T cells express a Vδ2 TCR, commonly Vγ9Vδ2, whereas this is less common among tissue-resident γδ T cells such as those in the skin, which more frequently use the Vδ1 TCR paired with gamma chains, for example often paired with Vγ4 in the gut.

γδ T cells play a critical role in immune surveillance, recognising malignant or transformed cells (such as cancer cells) through a pattern of stress markers and then exerting potent and selective cytotoxicity. γδ T cells can therefore act as orchestrators of an immune response. Modulation of these cells in situ provides the potential to increase immunogenicity even in tumours with low mutational load which have proven challenging with other immunotherapies. Recognition of tumours by γδ T cells is not dependent on any single tumour antigen and modulators of γδ T cells therefore have potential in a range of disease indications, including both haematological and solid malignancies. The recognition mechanism of γδ T cells is not MHC restricted. Vδ1 γδT-cells possess both adaptive as well as innate mechanisms of tumor cell recognition. Specifically, Vδ1 γδT-cell recognition of stressed or transformed cells relies on the engagement of their TCR and/or engagement of natural cytotoxicity receptors (NCRs), enabling innate tumor recognition while discriminating and sparing normal healthy cells.

The authors of WO2019147735 hypothesise that some γδ cells have pro-tumour activity or inhibit the anti-cancer immune response mediated by αβ T cells. The authors postulate that γδ T cells are immunosuppressants and therefore suggest they should be depleted, inhibited or blocked in a cancer setting with the aid of antibodies. However, it has been found that a positive correlation between γδ T cell infiltration and prognosis and/or survival in patients exists (see for example WO2021032963).

Compared to αβ TCR receptor/ligand interactions, understanding of vδ1 TCR receptor/ligand interactions are limited. Many antibodies which recognize vδ1 TCRs to date are mainly exploratory tools to probe this interaction. Such tools are typically crude, blocking antibodies which suggest TCR receptor/ligand interactions results in blocking, suppression or ablation of vδ1+ cells. For example, tool antibodies TS8.2 and TS-1 are employed as anti-γδ blocking antibodies in studies which suggest said antibodies reduce the cytotoxicity of vδ1 cells. These studies, combined with others, suggest use of such anti-vδ1 antibodies to favourably modulate the cytotoxicity of vδ1 cells in an in situ disease setting is inconceivable and there is therefore the need for antibodies which increase, not reduce, vδ1 cytotoxicity. WO2021032963 and WO2022175413 provide examples of such antibodies, and there is a need to further develop these to advance new treatment routes.

To exploit γδ T cells for immunotherapy requires either a means to expand the cells in situ or to harvest them and expand them ex vivo prior to re-infusion (for example see WO2017/072367 and WO2018/212808). However, there is still a need for activating agents that have the proven capacity to expand large numbers of γδ T cells.

Further, a binding or activating agent capable of preferentially targeting or binding or recognizing or specifically modulating or increasing the numbers of Vδ1+ cells in-situ may be highly desirable as a medicament.

Further, and given the predominate tissue-resident nature of Vδ1+ cells, an ideal medicament capable of modulating Vδ1+ would also exhibit fewer ‘off-target’ undesirable effects and rapid renal clearance.

Hence there is a need for improved medicaments specifically designed to target Vδ1+ cells and for the treatment of infections, autoimmune conditions, and cancer, Specifically, there is a need for medicaments that can be administered to ameliorate signs and symptoms of disease by specifically binding Vδ1+ cells, targeting Vδ1+ cells, specifically activating Vδ1+ cells, specifically enhancing proliferation and/or cytotoxicity activity Vδ1+ cells, or specifically blocking activation of Vδ1+ cells.

Various formats of bispecific and multispecific antibodies have been developed for a variety of therapeutic uses. Bi- and multispecific antibodies can be divided into separate, although overlapping, classes based on the types of biological targets and modes of action. For example, such multispecific antibodies can be divided into classes such as cytotoxic effector cell redirectors (also known as bispecific, T-cell-recruiting antibodies, bispecific T-cell engagers, TCEs, or BiTEs) and dual immunomodulators (DIs).

TCEs (T-cell engagers) are intended to enhance the patient's immune response to tumours by targeting T cells to tumour cells or vice versa, and work by targeting a first epitope of a T-cell receptor complex of a T-cell (usually CD3) and a second epitope, which is a cancer antigen or a cancer-associated antigen, such as a tumour associated antigen (TAA). Such antibodies colocalize tumour cells and T-cells to promote tumour cell killing. TCEs such as BiTEs are generally provided in an scFv format, although other formats have been provided. For example, BiKEs are similar to BiTEs, but they target CD16 on NK cells, rather than CD3.

However, whilst the use of such TCEs to treat cancer remain a compelling concept, to date and even after 30 years of concerted efforts to advance TCEs in early clinical development, many of such bispecific antibodies have exhibited lacklustre safety, efficacy and manufacturability profiles. Indeed, as of December 2023, only eight TCE are currently approved and marketed in the US, despite years of research and development, all of which are CD3 T-cell engagers.

Bispecific, T-cell-recruiting antibodies are discussed in Lejeune et al., 2020, Front Immunol., 11:762. However, the existing bispecific antibodies in this category, in particular those that recruit T-cells via CD3 binding, have significant off-target effects that result in severe adverse effects, given the potency of the CD3 antigen as signal transducer and its ubiquity in a patient's T-cell population. Hence for many CD3 targeting bispecific examples (such as Catumaxomab—now withdrawn), systemic delivery (e.g. intravenous) is not a realistic possibility. Instead, more contained delivery such as intra-operative, intra-peritoneal, intra-abdominal etc. is more often contemplated. This thereby limits optionality and use of said bispecifics as medicaments. Indeed, even for effector-attenuated anti-CD3 antibodies (i.e. a CD3 targeting T-cell complex engager but not a bispecific), the associated toxicity makes I.V. delivery challenging. For example, to limit exposure and reduce toxicity, the anti-CD3 antibody Foralumab is now most often being contemplated for oral delivery (e.g. in treatment of gut disease).

It is often stated that many of the current setbacks observed with such TCEs in early clinical trials are due to the high-affinity T-cell complex binding domains employed. Further, it has been proposed that this is because those designing these TCEs had not given due consideration to the low affinity of natural TCR-complex binding events they were hampered by severe dose-limiting toxicities resulting in prohibitively narrow therapeutic windows. Related to this it has been highlighted that many early TCE drug developers relied on three anti-CD3 T-cell complex binding domains derived from OKT3, SP34, and UCHT1. And these original binding domains all bind with a relatively high affinity in the single to low double-digit nM range equating to roughly to 1,000-fold higher affinity than a natural binding event. In turn it has been proposed that this can result in profoundly different (and often unfavourable) effects on the activation of T-cells compared to natural binding of the T-cell receptor complex. For example, TCE developers using platforms based on the higher affinity OKT3 may be confounded by the fact the OKT3 is apoptotic to T-cells in the presence of IL-2. For these reasons it has become apparent that lower affinity T-cell complex binding can be an important consideration for determining the design parameters of T-cell engaging bispecific antibody therapeutics.

Another issue when designing said TCEs, is the need to attenuate Fc function. Indeed, typically TCEs require the complete suppression of the Fc-mediated effector functions in order to maximize therapeutic efficacy and to minimize off-target toxicity because binding of Fc to Fc gamma receptor (FcγR) leads to activation of immune effector cells. In reality, the majority of the CD3-targeting bispecific antibodies currently in clinical practice have Fc domains with reduced binding activity to FcγR or are bispecific fragments intentionally without the Fc region. It would generally be expected that a TCE with unattenuated Fc function would induce an antibody-dependent cell-mediated cytotoxicity (ADCC) effect and thereby deplete the population of γδ T-cells recognized by the antibody. However, and again, by attenuating such functionality to avoid toxicity/safety complexities, one may also attenuate a potentially important efficacy angle too e.g. by engaging CD16+ or CD32+ or CD64+ immune cells, or by reducing half-life of the bispecific (e.g. if employing smaller bispecific antibody fragments such as BITEs). Methods of reducing the interaction of the FcγR and the TCE (such as using an IgG format designed to reduce said interaction) would be expected to reduce Fc-mediated immobilization of the TCE and reduce TCR clustering by cross-linking with the immobilized TCE.

To address some but not all of these complications, many companies such as Xencor® (Pasadena, CA), Macrogenics® (Gaithersburg, MD) and Genentech® (San Francisco, CA) have more recently reported reducing the binding affinity of the T-cell receptor complex binding arms in their respective TCE platforms. However, reducing the affinities of said binding may result in less effective efficacy and less optionality in terms of TCE design and functionality. For example, it is now demonstrated that affinity of the binding domains in such TCEs drives distribution profile in vivo. Specifically, it is typically observed that TCE distribution is biased towards its highest affinity target. Hence, by reducing the affinity of a TCE binding domain to the T-cell complex, it is typical to then bias distribution away from T-cells; the very cells needed to drive efficacy of such TCEs. It is partly for such reasons that TCE therapeutic windows have been termed ‘prohibitively narrow’.

Hence there is a need for improved TCE-type medicaments that address these problems with existing therapies and candidate therapies.

### SUMMARY

The present disclosure relates to high-affinity antibodies that comprise multiple antigen-binding sites (“multispecific antibodies”), including an antigen-binding site for TCR delta variable 1 (Vδ1) and an antigen-binding site for PD-L1. More specifically, the present disclosure relates to multispecific antibodies comprising a Fab region and an Fc region, wherein the Fab region comprises a binding site specific for an epitope of the variable delta 1 (Vδ1) chain of a γδ T cell receptor (TCR); and the Fc region comprises a binding site specific for PD-L1. In some embodiments, the multispecific antibodies are Vδ1 agonists and/or PD-L1 antagonists.

The multispecific antibodies of the present disclosure are in the format known as “mAb² antibodies or “mAb squared antibodies”, which are antibodies comprising an Fc region that has been engineered to contain antigen-binding loops in its CH3 domain—this modified Fc region is termed an “Fcab” (Fc with antigen binding). The resulting Fcabs can be rapidly inserted into a natural IgG antibody format to create tetravalent mAb² bispecific antibodies that bind to two different antigens. The mAb² antibody therefore further comprises a Fab region, comprising a VH-VL domain pair providing an antigen-binding site. mAb² molecules of the present disclosure comprise a PD-L1 binding Fcab and an vδ1-binding Fab.

The antibodies of the present disclosure have an advantageous functional profile:
In particular, the antibodies of the present disclosure are useful for the activation of Vδ1 T-cells. Although they may cause downregulation of the TCRs on T-cells to which they bind, they do not cause Vδ1 T-cell depletion, but rather they stimulate the T-cells.
Activation of Vδ1 T-cells in turn triggers release of inflammatory cytokines such as INFγ and TNFα to promote immune licensing and activate non-Vδ1 immune cell subsets like natural killer (NK) cells and αβ T cells resulting in further antitumor activity mediated by non-Vδ1 lymphocytes
Surprisingly, antibodies having suitably high affinity for TRDV1 elicit increased Vδ1 T-cell killing and, unlike (for example) antibodies that target CD3, the provision of high affinity antibodies is possible without adverse effects associated with large-scale activation via CD3. In turn, the high affinity antibodies are able to induce strong immunostimulatory effects via tumour-infiltrating lymphocytes (TILs). This can be achieved with minimal exhaustion or killing of the Vδ1 cells. Therefore, the multispecific antibodies of the disclosure may be considered agonistic antibodies.
The multispecific antibodies of the disclosure are proposed to exert antitumor activity by directing blood and tumor resident Vδ1 T cells to target PD-L1+ tumor cells.
   In the blood, the multispecific antibodies of the disclosure are designed to crosslink the Vδ1 TCR upon Vδ1 γδ T cells with PD-L1 expressed upon normal immune cells to stimulate Vδ1 γδ T cell priming and proliferation and upregulation of key homing receptors, thereby promoting chemotaxis toward the tumor e.g. migration to the tumor microenvironment (TME). This effect is not seen or expected with other Vδ1×TAA bispecifics.
   In the tumor, the multispecific antibodies of the disclosure engage Vδ1 γδ T cells and PD-L1+ cancer cells and enhance innate functionality. It is proposed they crosslink the Vδ1 TCR on Vδ1 γδ T cells with PD-L1 expressed on tumor cells, enabling Vδ1 immunological synapse formation and Vδ1 TCR downregulation, triggering Vδ1 γδ T cell activation and subsequent cytokine production, proliferation, and subsequent Vδ1 γδ T cell tumor-targeted cytotoxicity through degranulation. This effect is not seen or expected with other Vδ1×TAA bispecifics.
This activation of blood and tumor resident Vδ1 γδ T cells also drives Vδ1 γδ T cell priming and proliferation as well as chemokine and cytokine release. For example, Vδ1 cytokine production mediated by multispecific antibodies of the disclosure recruits cells from the wider immune system (such as CD4 and CD8 αβ T cells, NK cells and monocytes) to the TME. Vδ1 production of IFNγ also increases local tumor PD-L1 expression, driving tumour cell lysis. Vδ1 chemokine production mediated by multispecific antibodies of the disclosure stimulates wider local immune responses such as activation of local antigen presenting cells and activation of local αβ T cells. This demonstrates the potential for the multispecific antibodies of the disclosure to elicit immunorchestration and a broader anti-tumor immune response. This effect is not seen or expected with other Vδ1×TAA bispecifics.
The activation of blood and tumor resident Vδ1 γδ T cells with the antibodies of the present disclosure also drives an increase in innate stress ligand receptor expression on Vδ1+γδ T cells. This enhances targeting of PD-L1 negative tumor cells and leads to tumor cell lysis, demonstrating the potential of the multispecific antibodies of the disclosure to elicit immunorchestration and a broader anti-tumor immune response for both PD-L1+ and PD-L1− tumor cells.
The primed Vδ1 γδ T cells in the blood can then undergo differentiation to cytotoxic Teff phenotype, memory T cells, or a mixed memory- & effector-like phenotype in the presence of tumour cells. This effect is not seen or expected with other Vδ1×TAA bispecifics.
Such effects would not be expected even from Vδ1×TAA bispecifics that may be deemed similar to PD-L1. For example, PD-L1 is more widely expressed than other TAAs (such as PD-1) and the expression profile is advantageous for the claimed therapeutic applications.
Such effects would also not be expected from Vδ1×TAA bispecifics that target a TAA not highly expressed in the periphery, such as EGFR. Targeting TAAs that are not highly expressed in the periphery mean such tumour free expansion effects would not be realised.
Decoupling functional activation of Vδ1 cells from blockade of PD-L1 pathways enables activation of key Vδ1 anti-tumor functionality while avoiding potential additional deleterious effects of off-tumor PD-L1 blockade, as well as providing a clear biological rationale for using Vδ1-PDL1 engagers in patients who are PD-L1 blockade resistant.
Previous attempts to generate PD-L1/Vδ1 bispecifics in other formats (for example Morrison body format with the anti-PD-L1 scFV hanging off the c terminal of the heavy chain) failed because the bispecific antibody was aggregating, and no meaningful data was collected. In contrast, the bispecific antibody comprising a Fab region comprises a binding site specific for an epitope of the variable delta 1 (Vδ1) chain of a γδ T cell receptor (TCR), and the Fc region comprises a binding site specific for PD-L1 was produced with less aggregation (see Example 24).

The Fcab regions comprised within the multispecific antibodies of the present disclosure have a smaller binding interface than typical monoclonal antibody binding sites. Where the Fab arms of a typical mAb are separated by a flexible hinge region, the binding sites of Fcabs form a compact antibody fragment with two binding sites situated close together. The two antigen binding sites of an Fcab are also spatially close to each other, as compared with those of a typical mAb. Based on this smaller binding interface and reduced flexibility of the two binding sites it is surprising that the multispecific antibodies of the disclosure (comprising Fcab regions) are able to bind to and inhibit PD-L1 with similar affinity and potency as a monoclonal antibody benchmark.

As described herein, there is a need for improved TCE-type medicaments that address these problems with existing therapies and candidate therapies including a platform that affords more optionality and does not rely on Fc-domain deactivation or attenuation, or on specific affinities to the TCR complex, and provides a safer therapeutic with reduced adverse side effects.

There is also a need for medicaments that engage γδ T cells and cancer cells, directing anti-tumor responses by enabling immunological synapse formation and subsequent γδ T cell activation and tumor-targeted cytotoxicity through degranulation. Medicaments that can activate both blood and tumor resident Vδ1 γδ cells are particularly needed, since therapeutics that drives peripheral γδ T cell proliferation as well as chemokine and cytokine release have the potential to elicit a broader anti-tumor immune response.

Dual immunomodulator antibodies (Dis) are antibodies that bind two distinct immunomodulatory targets. Immunomodulatory targets include, for example, the immune checkpoint inhibitors PD-L1, PD-1, OX40, CTLA-4, LAG-3, TIM-3, TIGIT and VISTA (see, for example, Qin et al., 2019, Molecular cancer, 18:155). Typical example compounds in this class combine the targeting of two immunomodulatory signalling pathways. Examples of such Dis typically comprise at least two binding domains wherein both domains respectively target epitopes on two separate target proteins or complexed proteins present on immune cells such as T-cells.

PD-L1 (programmed death-ligand 1) is an immune checkpoint inhibitor found on some normal cells and in higher-than-normal amounts on some cancer cells. PD-L1 is the ligand of PD-1 (which is a protein typically found on T cells). When PD-L1 binds to PD-1 it prevents the T cells from killing the PD-L1 presenting cells, this is a “checkpoint” to prevent T cells from killing healthy PD-L1 presenting cells. However, it also prevents T cells from killing cancer cells presenting PD-L1. PD-L1 is a popular target for anti-cancer treatments, since anti-cancer therapies which bind to PD-L1 can block PD-L1 binding to PD-1. This leaves T cells free to kill cancerous PD-L1 presenting cells, although there is a risk that healthy PD-L1 presenting cells are also killed. Although PD-L1 therapies can be effective, many patients do not respond to such therapies or become refractory over time. There is a need for therapies which enhance the ability of T cells to recognise and kill PD-L1 positive cancer cells and spare healthy PD-L1 positive cells. There is also a need for effective treatments to treat patients who do not respond to existing PD-L1 treatments or become refractory.

There is a need in the art to generate a DI which targets at least two immunomodulatory targets wherein one such target is to the TCR δ1 chain, to help overcome the immunosuppressive tumour microenvironment and turn it from “cold” to “hot”, promoting the accumulation of proinflammatory cytokines and T cell infiltration, and tumour cell killing. Existing DI approaches are being studied in an attempt to reduce or overcome the high toxicity associated with combination therapies (as well as improve efficacy over combination treatments), but many require careful monitoring and there has been mixed success rates. There remains a need to provide alternative immunomodulatory approaches for the treatment of conditions such as cancer, and the present disclosure provides this.

Hence there is need for improved multispecific immunomodulatory medicaments wherein at least one binding domain binds a T-cell and at least one second binding domain targets a TAA.

There is also a need for improved medicaments specifically designed to target Vδ1+ cells and for the treatment of infections, autoimmune conditions, and cancer. Specifically, there is a need for medicaments that can be administered to ameliorate signs and symptoms of disease by specifically binding Vδ1+ cells, targeting Vδ1+ cells, specifically activating Vδ1+ cells, specifically enhancing proliferation and/or cytotoxicity activity Vδ1+ cells, or specifically blocking activation of Vδ1+ cells.

According to a first aspect of the disclosure, there is provided a multispecific antibody comprising a Fab region and an Fc region, wherein the Fab region comprises a binding site specific for an epitope of the variable delta 1 (Vδ1) chain of a γδ T cell receptor (TCR); and the Fc region comprises an binding site specific for PD-L1.

In a second aspect of the disclosure, there is provided a polynucleotide sequence encoding a multispecific antibody of the disclosure. For example, there is provided a polynucleotide sequence encoding the anti-Vδ1 first binding domain comprising a sequence having at least 70% sequence identity with SEQ ID NO: 199 to 222, 224 to 247, 249 to 259 or 261 to 271.

In a third aspect of the disclosure, there is provided an expression vector comprising the polynucleotide sequence of the disclosure. There is also provided a host cell comprising a polynucleotide sequence of the disclosure or an expression vector of the disclosure. There is also provided a method for producing any multispecific antibody or antigen-binding fragment thereof of the disclosure, comprising culturing a host cell of the disclosure in a cell culture medium.

In a further aspect of the disclosure, there is provided a composition comprising a multispecific antibody of the disclosure. There is also provided a pharmaceutical composition comprising a multispecific antibody of the disclosure and a pharmaceutically acceptable diluent or carrier. Compositions and pharmaceutical compositions may optionally further comprise one or more additional therapeutically active agents.

In a further aspect of the disclosure, there is provided a kit comprising a multispecific antibody of the disclosure or a pharmaceutical composition of the disclosure, optionally comprising instructions for use and/or an additional therapeutically active agent.

In a further aspect of the disclosure there is provided a method of treating a disease or disorder in a subject, comprising administering to the subject a multispecific antibody of the disclosure, or a pharmaceutical composition of the disclosure. There is also provided a method of modulating an immune response in a subject, comprising administering to the subject a multispecific antibody of the disclosure, or a pharmaceutical composition of the disclosure. Administration of antibodies to a subject may be administration in a therapeutically effective amount.

In a still further aspect of the disclosure, there is provided a multispecific antibody of the disclosure, or a pharmaceutical composition of the disclosure, or a kit of the disclosure, for use in medicine. There is also provided the use of an a multispecific antibody of the disclosure in the manufacture of a medicament.

### BRIEF DESCRIPTION OF THE FIGURES

FIGS. 1A-1C: Phage selection rounds. (FIG. 1A) Phage selections round 1 to 3 for the ADT1-7 library. (FIG. 1B) Phage selections Round 1 to 3 for the ADT1-4 library. (FIG. 1C) Phage selections Round 1 to 3 for the ADT1-4 library with a selection strategy for isolating cyno cross-reactive binders.

FIGS. 2A-2C: A schematic overview of selection of matured antibodies by mammalian display. (FIG. 2A) Schematics of the flow sorting of ADT1-7 library. (FIG. 2B) Schematics of the flow sorting of ADT1-4 library 1 (human). (FIG. 2C) Schematics of the flow sorting of ADT1-4 library 2 (cyno).

FIGS. 3A-3C: Impact of S74L change on affinity. (FIG. 3A) ADT1-4 Lineage Mammalian Display Outputs: Human Dissociation off-rate (SPR) vs Human Antigen binding (Delfia ELISA, 0.4 nM Human Antigen) with LC 74S (open circle) or 74L (filled circle) usage indicated. Results highlight good fit between improved affinity by SPR and by antigen binding by Delfia ELISA. These results also highlight the improved binding conferred by variable domain S74L modification. (FIG. 3B) ADT1-4 Lineage Mammalian Display Outputs: Cyno Dissociation Off-Rate (SPR) vs Cyno Antigen Binding (Delfia ELISA, 10 nM Cyno Antigen) with LC 74S (open circle) or 74L (filled circle) usage indicated. (FIG. 3C) ADT1-4 Lineage Mammalian Display Outputs: Correlation between Human Antigen Dissociation Off-Rate (SPR) improvements vs Cyno Antigen Dissociation Off-Rates (SPR) plus LC 74S (open circle) or 74L (filled circle) usage indicated.

FIGS. 4A-4C: CDR3 usage and cross-sharing. (FIG. 4A) Example heavy chain and light chain cross-sharing between affinity matured antibodies in ADT1-4 (G04) lineage (0.4 nM Human TRDV1 ELISA results heat-map graded). This “heat map” demonstrates the cross-sharing of CDR3 sequences between different affinity matured antibodies in the ADT1-4 lineage, showing the affinity maturation process provided antibody sequences that do not have to be provided as specific heavy and light chain pairs. The results highlight the affinity matured antibodies can cross-share light chains and heavy chains and that differing LC/HC combinations generate equivalent or improved outcomes in a high-stringency antigen binding study. Also included in the figure is the ADT1-4 (G04) parent clone, (bottom left). (FIG. 4B) ADT1-4 Lineage Final Selection: CDR3 usage and cross-sharing+Cyno Antigen Binding ‘Heat Map’ versus starting parent ADT1-4 parent G04 mAb (bottom left). (FIG. 4C) ADT1-7 Lineage Final Selection: CDR3 usage and cross-sharing+Human Antigen Binding ‘Heat Map’ versus starting ADT1-7 parent E07 mAb (bottom left).

FIGS. 5A-5D: Fold enhancement in binding and for ADT1-4 linage compared to ADT1-4 parental G04). (FIG. 5A) Fold enhancement in recombinant human Vδ1 antigen binding. (FIG. 5B) Fold enhancement in recombinant cyno Vδ1 antigen binding. (FIG. 5C) Fold enhancement in primary Vδ1 MFI. (FIG. 5D) Fold enhancement in PEER Vδ1 cell line MFI.

FIGS. 6A-6C: Fold enhancement in binding and for ADT1-1 linage compared to ADT1-4 parental E07). (FIG. 6A). Fold enhancement in recombinant human Vδ1 antigen binding. (FIG. 6B). Fold enhancement in primary Vδ1 MFI. (FIG. 6C). Fold enhancement in PEER Vδ1 cell line MFI.

FIGS. 7A-7C. Fold improvements in human (and cyno) antigen binding over parental clones. (FIG. 7A) Fold improvement in binding to human antigen. DELFIA ELISA signal of affinity matured clones vs parent ADT1-4 (G04), 0.4 nM of recombinant human L1 antigen (containing human Vδ1 TCR antigen). (FIG. 7B) Fold improvement in binding to cyno antigen (DV1/GV77 containing Cyno SEQ ID NO: 308 (mature, minus leader)). DELFIA ELISA signal of affinity matured clones vs parent ADT1-4 (G04), 0.4 nM of recombinant human L1 antigen. (FIG. 7C) Fold improvement in binding to human antigen. DELFIA ELISA signal of affinity matured clones vs parent ADT1-7 (E07), 0.4 nM of recombinant human L1 antigen.

FIGS. 8A-8C. Fold improvements in KD for human (and cyno) antigen over parental clones. (FIG. 8A) Fold improvement in human antigen KD for ADT1-4 (G04) lineage. (FIG. 8B) Fold improvement in cyno antigen KD for ADT1-4 (G04) lineage. (FIG. 8C) Fold improvement in human antigen KD for ADT1-7 (E07) lineage.

FIGS. 9A-9F. Binding affinity analysis (KD by SPR) to human Vδ1 antigen. (FIG. 9A) Surface plasmon resonance for ADT1-4 lineage. (FIG. 9B) Surface plasmon resonance for ADT1-7 lineage. (FIG. 9C) KD values and fold change against parental clone for ADT1-4 lineage. (FIG. 9D) KD values and fold change against parental clone for ADT1-7 lineage. (FIG. 9E) Fold change of KD against parental clone for ADT1-4 lineage. (FIG. 9F) Fold change of KD against parental clone for ADT1-7 lineage.

FIGS. 10A-10B. Binding affinity analysis (KD by SPR) to cyno antigen. (FIG. 10A) Surface plasmon resonance for ADT1-4 lineage for cyno antigen. (FIG. 10B) KD values for ADT1-4 lineage for cyno antigen.

FIGS. 11A-11D. Binding Affinity to cell surface Vδ1 TCR (EC50 for binding to cell surface Vδ1). (FIGS. 11A, 11B) The level of binding by the Vδ1 mAbs to two γδ T cell donors, ATS006 (FIG. 11A) and TS164 (FIG. 11B). (FIG. 11C) Bar chart representing the average 50% binding values from the ADT1-4 and ADT1-7 clones binding to Vδ1-positive γδ T cell, represented as the mean two donors, with these values provided in tables with % improvement. (FIG. 11D) Table summarizing the IC50s plotted in (FIG. 11A) & (FIG. 11B), and Vδ1-negative cell types including HEK293A, Raji cells and various leukocyte subsets with primary blood mononuclear cells. For Vδ1-positive γδ T cell, data is represented as the mean two donors.

FIGS. 12A-12G. TCR downregulation. (FIGS. 12A, 12B) ADT1-4 lineage (FIG. 12A) and ADT1-7 lineage (FIG. 12B). (FIG. 12C) Average TCR downregulation—IC50 results from two GD cell donors. (FIGS. 12D, 12E) Fold TCR downregulation improved from the parental ADT1-4 clone (FIG. 12D) and the ADT1-7 clone (FIG. 12E). (FIG. 12F) 50% effect values from (FIG. 12C), with the percentage improvement calculated from the respective parents of ADT1-4 (upper) and ADT1-7 (lower). (FIG. 12G) 50% effect values from (FIG. 12C) with the fold improvement calculated from the respective parents of ADT1-4 (upper) and ADT1-7 (lower).

FIGS. 13A-13F. Vδ1 monoclonal antibodies on γδ activation measured by CD107a expression. (FIGS. 13A, 13B) ADT1-4-2 clone in cells GD cells alone (FIG. 13A) or with THP-1 cells (FIG. 13B). (FIGS. 13C, 13D) ADT1-7-3 clone in cells GD cells alone (FIG. 13C) or with THP-1 cells (FIG. 13D). (FIG. 13E) Table representing the percentage increase in γδ CD107a expression from cocultured cells treated with the highest concentration of Vδ1 mAb compared to untreated non-cocultured γδ cells. (FIG. 13F) Table representing the percentage increase in γδ CD107a expression from cocultured cells treated with the highest concentration of Vδ1 mAb compared to untreated, co- and non-cocultured γδ cells.

FIGS. 14A-14F. Vδ1 monoclonal antibodies on γδ activation measured by CD25 expression. (FIGS. 14A, 14B) ADT1-4-2 clone in cells GD cells alone (FIG. 14A) or with THP-1 cells (FIG. 14B). (FIGS. 14C, 14D) ADT1-7-3 clone in cells GD cells alone (FIG. 14C) or with THP-1 cells (FIG. 14D). (FIG. 14E) Table representing the percentage increase in γδ CD25 expression from cocultured cells treated with the highest concentration of Vδ1 mAb compared to untreated non-cocultured γδ cells. (FIG. 14F) Table representing the percentage increase in γδ CD25 expression from cocultured cells treated with the highest concentration of Vδ1 mAb compared to untreated, co- and non-cocultured γδ cells.

FIGS. 15A-15C. Cyno TCR Downregulation. (FIG. 15A) Comparison of ADT1-4-2 and ADT1-4 capability to engage and reduce the expression of VD1 on cynomolgus γδ-T cells. (FIG. 15B) Percentage of cell surface expression of VD1 upon treatment with ADT1-4-2. (FIG. 15C) individual EC50 value for the different donors combined with the mean and standard deviation.

FIGS. 16A-16D. Quantification of live THP-1 cell numbers after 24 hours co-culture with γδ T-cells in the presence of Vδ1 mAbs or controls. (FIG. 16A) THP-1 cell killing assay for ADT1-4 clones. (FIG. 16B) THP-1 cell killing assay for ADT1-7 clones. (FIG. 16C) Mean EC50s in THP-1 killing assay for ADT1-4 and ADT1-7 clones. (FIG. 16D) Table summarizing the EC50s plotted in (FIG. 16C).

FIG. 17. Vδ1 monoclonal antibodies on antibody-dependent cellular cytotoxicity (ADCC).

FIG. 18. Vδ1 monoclonal antibodies on complement-dependent cytotoxicity (CDC).

FIG. 19. Healthy cell sparing of clone ADT1-4-2.

FIGS. 20A-20J. Effect of anti-Vδ1 antibodies on TIL populations from primary tumour biopsies. (FIGS. 20A, 20B) Show % decrease in Vδ1 TCR expression on total tumour infiltrating-γδ T cells following 48 (FIG. 20A) or 72 (FIG. 20B) hours mAb stimulation in two separate donors, confirming target engagement in each case. (FIG. 20C) Shows enhanced expression of both CD25 and Ki-67 on Vδ1+ T cells following 48-hour stimulation with ADT1-4-2 compared with stimulation with IgG1 isotype control or ADT1-4. (FIG. 20D) Shows substantial fold increases in IFN-γ production by TILs stimulated with ADT1-4-2 or ADT1-7-3 for 72 hours in the presence of 50 ng/ml IL-15. (FIG. 20E) Shows that stimulation of TILs with ADT-1-4-2 or ADT1-7-3 did not enhance secretion of type 17-associated cytokines IL-6 or IL-17 at this timepoint. (FIGS. 20F, 20G) Show the % decrease in Vδ1 TCR expression on total tumour infiltrating-γδ T cells following mAb stimulation at 24 (FIG. 20F) or 72 (FIG. 20G) hours in two individual donors, confirming target engagement in TILs isolated by enzymatic digestion. (FIG. 20H) Shows dose-dependent enhanced expression of Ki-67 on γδ T cells following 72 hours stimulation with ADT1-4-2. (FIGS. 20I, 20J) Show the fold increase in IFN-γ produced by TILs isolated from two individual donors by enzymatic digestion and stimulated with ADT1-4-2 at a concentration of 6.66 nM in the presence of 2 ng/ml IL-15 at 24 (I) or 72 (FIG. 20J) hours. In all cases, the concentration of the control anti-RSV and parent ADT1-4 are matched to highest concentration used in study (i.e. a conc. of 6.66 nM for FIGS. 20A, 20C, 20F, 20G, 20H, 20I, 20J and a concentration of 66.6 nM for FIGS. 20B and 20E).

FIGS. 21A-21E. Effect of anti-Vδ1 antibodies on TIL populations from primary tumour biopsies. (FIG. 21A) Shows enhanced expression of CD25 and Ki-67 in γδ T cells stimulated for 10 days with ADT1-4-2. (FIG. 21B) Shows a substantial increase in Perforin⁺ Granzyme B⁺ γδ T cells following 10 days stimulation with ADT1-4-2. (FIG. 21C) Shows considerably enhanced expression of Granzyme B and Perforin by both CD8⁺ and CD8⁻ αβ T cells following 10 days stimulation of tumour-infiltrating Vδ1⁺ T cells with ADT1-4-2. (FIG. 21D) Shows markedly increased production of IFN-γ, and moderately increased production of IL-17 and IL-6 by lung tumour-derived TILs following stimulation with ADT1-4-2. (FIG. 21E) Demonstrates enhanced production of the chemokines CCL2, CCL4 and CXCL10 by TILs following 10 days stimulation with ADT1-4-2. In all cases, control anti-RSV and parent ADT1-4 matched to same concentration as affinity matured ADT1-4-2.

FIGS. 22A-22B: Sequences of ADT1-4 lineage clones (light chains)

FIGS. 23A-23B: Sequences of ADT1-4 linage clones (heavy chains).

FIG. 24: Sequences of ADT1-7 lineage clones (light chains).

FIG. 25: Sequences of ADT1-7 lineage clones (heavy chains)

FIGS. 26A-26B: Further evidence of non-depletion and CD3 downregulation in both blood-derived and tumour-associated Vδ1 T cells. (FIG. 26A) shows the vδ1 TCR MFI upon antibody stimulation as an indication of mAb target engagement on blood derived Vδ1 T cells. (FIG. 26B) shows the MFI of CD3 expression on positively gated blood derived vδ1 cells. Stimulation with the vδ1 antibody engaged vδ1 cells and resulted in down-regulation of both vδ1 and CD3 on vδ1 cells.

FIG. 27: ADCC reporter bioassay shows no ADCC as a result of the anti-vδ1 antibodies. Target cells, i.e. γδ cells, were incubated with the ADCC bioassay effector cells in presence of anti-vδ1 antibodies, anti-vδ1 LAGA antibodies (Fc disabled), and RSV Isotype control. Luminescence signal was recorded as relative light units (RLU), and fold induction was calculated as described in the methods. N=2 γδ donors (performed in technical duplicates) for “anti-vδ1 antibody”, “anti-vδ1 LAGA antibody”, “RSV”, “OKT3”. N=1 Raji cell lines for the “Rituximab+Rajis” condition (in technical duplicates) and n=1 γδ donors for the “anti-vδ1 antibody+Effector” and “anti-vδ1 LAGA antibody+Effector” conditions (performed in technical duplicate and singlicate, respectively). Effector:target ratio at 3:1.

FIGS. 28A-28D: A table showing cross referenced CH3 residue numbering. The table is adapted from IMGT.org. The positions of the modified amino acids as per IMGT (standard), IMGT (exon), EU, and Kabat numbering referred to elsewhere herein are highlighted by shading. Please note a necessary but unconventional position nomenclature to describe an additional amino acid insert found in some example constructs is also indicated—see below position 21.1 (IMGT exon column)/361.1 (EU column)/384.1 (Kabat column)/17.1 (IMGT standard column).

FIGS. 29A-29F: Protein A capture for human PD-L1. The binding affinity of the antibodies to recombinant human PD-L1 established by SPR analysis using a Reichert 4SPR instrument (Reichert Technologies).

FIGS. 30A-30B: Protein A capture (human Vδ1 and PD-L1). The binding affinity of the antibodies to recombinant human PD-L1 and human Vδ1 established by SPR analysis using a Reichert 4SPR instrument (Reichert Technologies), focusing on the effect of altering the anti-Vδ1 Fab.

FIG. 31: Biacore SPR analysis. The binding affinity of the antibodies to recombinant human and cynomolgous Vδ1 and PD-L1 established by SPR analysis using a Biacore 8K+(Cytiva) with a Carboxymethylated dextran coated CM5 chip.

FIG. 32: On cell binding to ADX011 skin-Vδ1s. Skin derived Vδ1 γδT cells and PD-L1 positive tumor cells (SW900, MDA-MB-231) and Vδ1−/PD-L1− negative control cells (HEK293) were assayed to determine the specificity and on cell binding affinity of PD-L1/Vδ1 bispecific antibodies.

FIGS. 33A-33E: Vδ1 degranulation. The Vδ1/PD-L1 bispecific antibodies were assayed for their ability to induce the degranulation of primary Vδ1-positive γδ T-cells in a PD-L1 dependent manner (FIGS. 33A-33C). The effect of different anti-Vδ1 affinities was also assayed (FIGS. 33D-33E).

FIG. 34: Vδ1 γδT cell-dependent Cellular Cytotoxicity (TDCC) assays. The Vδ1/PD-L1 bispecific antibodies with different anti-Vδ1 affinities ranging from <1 nM to 60 nM were assayed for their ability to enhance the cytolytic ability of primary Vδ1-positive γδ T-cells in a PD-L1 dependent manner.

FIG. 35: TDCC antibody titration. Using ADT5-pre expanded PBMC to assay blood-derived Vδ1 γδT cell cytolytic potential.

FIG. 36: TDCC Ab titration. To assay skin-derived Vδ1 γδT cell cytolytic potential.

FIG. 37: Binding assessment of ADT1-4-2/ADT5-1 and ADT1-4-2/ADT5-2 using Retrogenix Cell Microarray Technology. This assay demonstrates pharmacokinetics is not anticipated to be different for ADT5-1 & ADT5-2.

FIG. 38: Safety and PD-L1 related toxicities. This figure demonstrates that multispecific antibodies of the disclosure block the PD-1/PD-L1 axis within 0.5-1 log of SoC. It also demonstrates that multispecific antibodies of the disclosure are Vδ1 agonists in the pM range and PD-L1 antagonists in the nM range.

FIGS. 39A-39B: Fold induction curve. A PD-1/PD-L1 reporter assay (Promega) was utilised to assess the ability of Vδ1/PD-L1 antibodies to block the PD-1/PD-L1 axis.

FIGS. 40A-40C: Pharm Sci/CMC: DPD Manufacturability. Protein precipitation, the aggregation during storage and the relative solubility and of ADT5-1 and ADT5-2 were tested using 5 different buffers. Buffer 1: 50 mM Histidine+20 mM Acetate, 100 mM Arginine HCl, pH 5.5; Buffer 2: 50 mM Histidine, 100 mM Arginine HCl, pH 5.5; Buffer 3: 20 mM Acetate, 100 mM Arginine HCl, pH 5.5; Buffer 4: 50 mM Histidine, 20 mM Acetate, 100 mM NaCl, pH 5.5; Buffer 5: 50 mM Histidine, 20 mM Acetate, 7% Sucrose, pH 5.5.

FIGS. 41A-41B: Stimulation with antibodies of the disclosure enhances blood Vδ1 proliferation and priming.

FIGS. 42A-42D: Frequency of Vδ1, Ki-67, NKG2D and CD27 in PBMCs following a 7-day treatment with TAK-505.

FIG. 43: Mean Vδ1 γδ T cell counts and fold change in Vδ1 γδ T cell growth following a 7-day treatment with 10 pM TAK-505.

FIGS. 44A-44B: Concentration of IFN-γ, GM-CSF, IL-17A, IL-10, IP-10, MIP-1α and IL-8 by skin-derived Vδ1 cells stimulated with TAK-505 or control articles during co-culture with PD-L1 positive tumour cell targets A375. Data from one donor (TBOS036) is shown, that is representative of the other tested donors.

FIGS. 45A-45C: Stimulation with antibodies of the disclosure. (FIG. 45A) Stimulation with antibodies of the disclosure enhances blood Vδ1 priming. (FIG. 45B) Stimulation with antibodies of the disclosure enhances blood Vδ1 tumor homing. (FIG. 45C) Stimulation with antibodies of the disclosure increases innate recognition of tumor cells.

FIG. 46: Activation by bispecific Vδ1 engagers enhances innate recognition of TAA-tumor cells by Vδ1 γδ cells. This figure depicts coculture or triculture assays consisting of tissue-derived Vδ1 cells in culture with tumor cell targets, whereby Vδ1 cells are cultured with either PD-L1 positive tumor cells, PD-L1 negative tumor cells or a 50/50 mix of both PD-L1 positive and negative tumour cells.

FIGS. 47A-47D: Depicts alternative PD-L1 multispecific formats as described in Example 24.

FIG. 48: Sequences of PD-L1 binding domains (IgG1 CH1-CH2-CH3 showing the sequence alignments and changes to IgG1 CH3 (EU numbering).

FIG. 49: Vδ1/PD-L1 bispecific antibodies differentiate peripheral Vδ1 γδ T cell phenotype into a mixed memory- & effector-like phenotype.

FIGS. 50A-50C: Re-priming with Vδ1/PD-L1 bispecific antibodies promote additional activation and proliferation of primed Vδ1 γδ T cells when challenged with PD-L1⁺ tumor cells.

FIG. 51: Re-priming with Vδ1/PD-L1 bispecific antibodies promotes restimulation of innate stress ligand receptor expression in primed Vδ1 γδ T cells when challenged with PD-L1⁺ tumor cells.

FIGS. 52A-52C: Vδ1/PD-L1 bispecific antibodies drive immune cell infiltration and normal tissue targeting using healthy and tumor organ-on-a-chip models.

### DETAILED DESCRIPTION

The present disclosure provides multispecific antibodies and fragments and variants thereof that specifically bind the T cell receptor of gamma delta T cells and specifically bind to PD-L1.

More specifically, the present disclosure relates to high-affinity antibodies that comprise multiple antigen-binding sites, including an antigen-binding site for TCR delta variable 1 (Vδ1) and an antigen-binding site for PD-L1 (“multispecific antibodies”).

In some embodiments, the multispecific antibodies of the present disclosure are Vδ1 agonists and PD-L1 antagonists. In some embodiments, binding of the multispecific antibodies of the present disclosure to Vδ1 causes activation of γδ cells (see “activating epitope”). In some embodiments, binding of the multispecific antibodies of the present disclosure to PD-L1 causes inhibition of the PD-L1/PD-1 axis, for example via blocking PD-L1 binding to PD-1. In some embodiments, the multispecific antibody comprises TCR Vδ1 binding domain that is a Vδ1 agonist. In some embodiments, the multispecific antibody comprises a PD-L1 binding domain that is a PD-L1 antagonist.

The multispecific antibodies of the present disclosure are in mAb² format and comprise a Fc region that has been engineered to contain antigen-binding loops in its CH3 domain—this modified Fc region is termed an “Fcab”. The mAb² antibody further comprises a Fab region, comprising a VH-VL domain pair providing an antigen-binding site. mAb² molecules of the present disclosure comprise a PD-L1 binding Fcab and an vδ1-binding Fab.

More specifically, the present disclosure relates to the provision and characterisation of multispecific antibodies, for example antibodies prepared according to a selection procedure beginning from parental anti-Vδ1 antibodies, such as the parental antibodies referred to herein as G04, E07, C08, B07, C05, E04, F07, G06, G09, B09, G10 and E01. The present disclosure relates in particular to multispecific antibodies derived from G04 and E07.

### Definitions

Unless defined otherwise, all technical and scientific terms used herein have the meaning commonly understood by a person skilled in the art to which this disclosure belongs. As used herein, the following terms have the meanings ascribed to them below.

Gamma delta (γδ) T cells represent a small subset of T cells that express on their surface a distinct, defining T Cell Receptor (TCR). This TCR is made up of one gamma (γ) and one delta (δ) chain. Each chain contains a variable (V) region, a constant (C) region, a transmembrane region and a cytoplasmic tail. The V region contains an antigen-binding site. There are two major sub-types of human γδ T cells: one that is dominant in the peripheral blood and one that is dominant in non-haematopoietic tissues. The two sub-types may be defined by the type of δ and/or γ present on the cells. For example, γδ T cells that are dominant in peripheral blood primarily express the delta variable 2 chain (Vδ2). γδ T cells that are dominant in non-haematopoietic tissues (i.e. are tissue-resident) primarily express the delta variable 1 chain. References to “Vδ1 T cells” or “Vδ1+ T cells” refer to γδ T cells with a Vδ1 chain, i.e. Vδ1⁺ cells.

References to “delta variable 1” may also referred to as Vδ1 or Vd1, while a nucleotide encoding a TCR chain containing this region or the TCR protein complex comprising this region may be referred to as “TRDV1”. Antibodies or antigen-binding fragments thereof which interact with the Vδ1 chain of a γδ TCR, and also interact with PD-L1 are all effectively antibodies or antigen-binding fragments thereof which bind to Vδ1 and may referred to as “anti-TCR delta variable 1 antibodies or antigen-binding fragments thereof” or “anti-Vδ1 antibodies or antigen-binding fragments thereof” or “anti-TRDV1 antibodies or antigen-binding fragments thereof” or “anti-Vδ1x PD-L1 antibodies or antigen-binding fragments thereof”.

Additional references are made herein to other delta chains such as the “delta variable 2” chain. These can be referred to in a similar manner. For example, delta variable 2 chains can be referred to as Vδ2, while a nucleotide encoding a TCR chain containing this region or the TCR protein complex comprising this region may be referred to as “TRDV2”. In preferred embodiments multispecific antibodies or antigen-binding fragments thereof which interact with the Vδ1 chain of a γδ TCR, do not interact with other delta chains such as Vδ2. In the disclosure, the antibodies are specific to TRDV1 and do not bind to TRDV2 (SEQ ID NO: 310) or other antigens present on a gamma delta T-cell receptor, such as TRDV3 (SEQ ID NO: 311).

References to “gamma variable chains” are also made herein. These may be referred to as γ-chains or Vγ, while a nucleotide encoding a TCR chain containing this region or the TCR protein complex comprising this region may be referred to as TRGV. For example, TRGV4 refers to Vγ4 chain. In a preferred embodiment, multispecific antibodies or antigen-binding fragments thereof which interact with the Vδ1 chain of a γδ TCR, do not interact with gamma chains such as Vγ4 (e.g. SEQ ID NO: 309). The antibodies also do not bind or interact with other domains found within a γδ TCR, such as TRDJ, TRDC, TRGJ or TRGC

The term “T-cell receptor complex” is the complex of proteins comprising the “T-cell receptor” (or “TCR”) found on the surface of T-cells responsible for recognising a variety of antigens. The T-cell receptor complex comprises either the alpha and beta chains of the T-cell receptor, or in the case of gamma delta T cells, the gamma and delta chains of the T-cell receptor, and up to 6 additional chains or more, such as CD3δ, CD3γ, CD3ε and CD3ζ, although the precise makeup of T-cell receptor complexes can vary. The T-cell receptor complex mediates intracellular signalling in the T-cell, which may lead to T-cell activation.

The term “antibody” includes any antibody protein construct comprising at least one antibody variable domain comprising at least one antigen-binding site (ABS). Antibodies include, but are not limited to, immunoglobulins of types IgA, IgG, IgE, IgD, IgM (as well as subtypes thereof). The overall structure of Immunoglobulin G (IgG) antibodies assembled from two identical heavy (H)-chain and two identical light (L)-chain polypeptides is well established and highly conserved in mammals (Padlan (1994) Mol. Immunol. 31:169-217).

A conventional antibody or immunoglobulin (Ig) is a protein comprising four polypeptide chains: two heavy (H) chains and two light (L) chains. Each chain is divided into a constant region and a variable domain. The heavy (H) chain variable domains are abbreviated herein as VH, and the light (L) chain variable domains are abbreviated herein as VL. These domains, domains related thereto, and domains derived therefrom, may be referred to herein as immunoglobulin chain variable domains. The VH and VL domains (also referred to as VH and VL regions) can be further subdivided into regions, termed “complementarity determining regions” (“CDRs”), interspersed with regions that are more conserved, termed “framework regions” (“FRs”). The framework and complementarity determining regions have been precisely defined (Kabat et al. Sequences of Proteins of Immunological Interest, Fifth Edition U.S. Department of Health and Human Services, (1991) NIH Publication Number 91-3242). There are also alternative numbering conventions for CDR sequences, for example those set out in Chothia et al. (1989) Nature 342: 877-883 or as summarized by IMGT.org. In a conventional antibody, each VH and VL is composed of three CDRs and four FRs, arranged from amino-terminus to carboxy-terminus in the following order: FR1, CDR1, FR2, CDR2, FR3, CDR3, FR4. The conventional antibody tetramer of two heavy immunoglobulin chains and two light immunoglobulin chains is formed with the heavy and the light immunoglobulin chains inter-connected by e.g. disulphide bonds, and the heavy chains similarly connected. The whole antibody thus comprises two Fabs, each Fab comprising a VH-VL domain pair. The heavy chain constant region includes three domains, CH1, CH2 and CH3. The light chain constant region is comprised of one domain, CL. The variable domain of the heavy chains and the variable domain of the light chains are binding domains that interact with an antigen. The constant regions of the antibodies typically mediate the binding of the antibody to host tissues or factors, including various cells of the immune system (e.g. effector cells) and the first component (C1q) of the classical complement system.

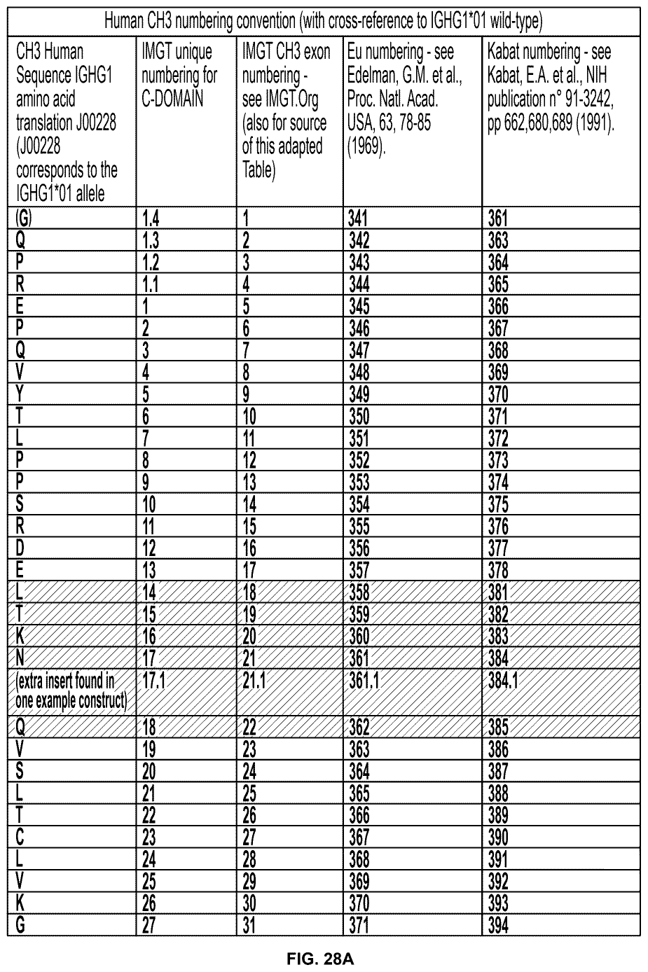

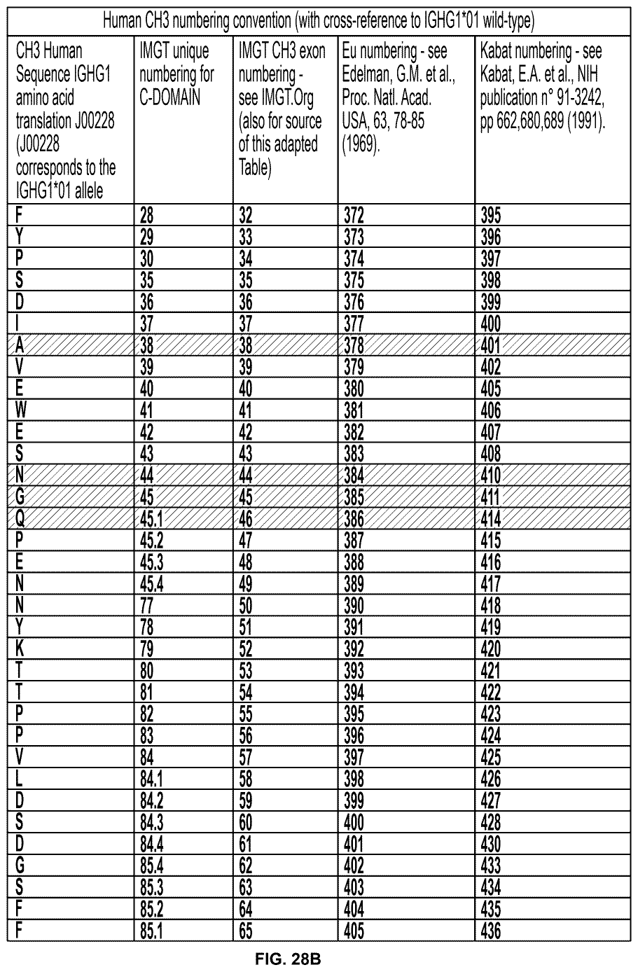

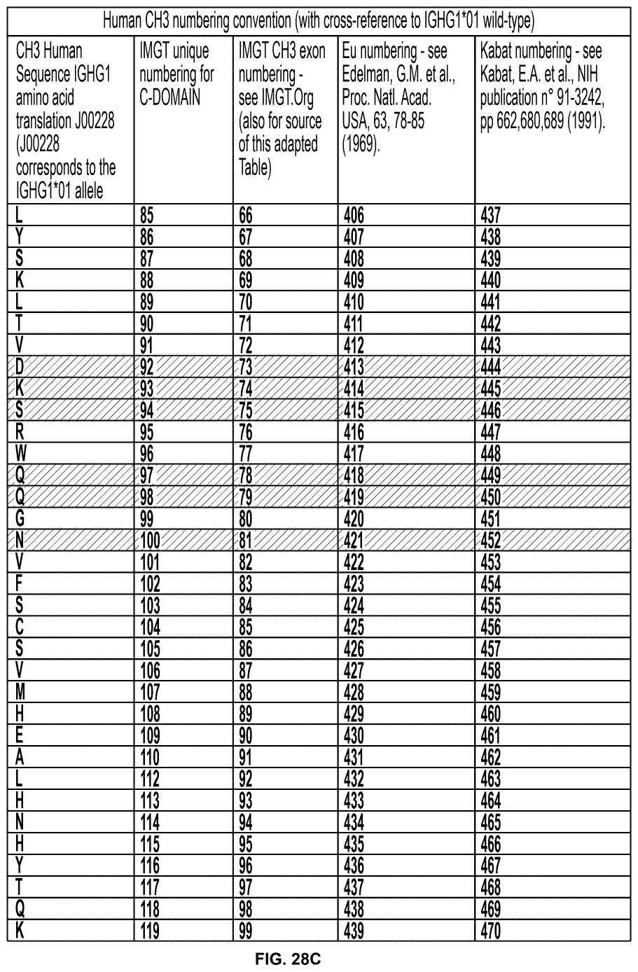

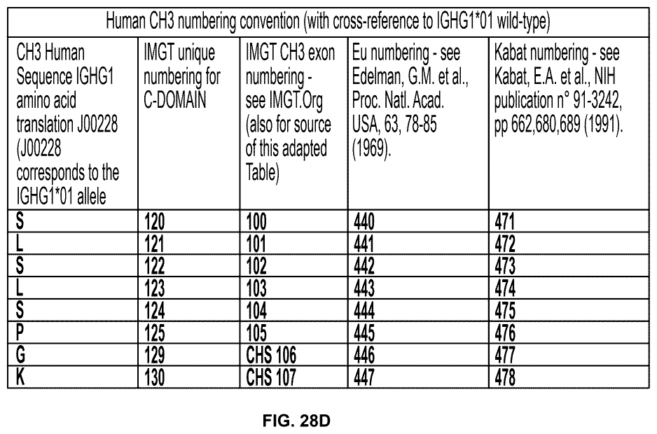

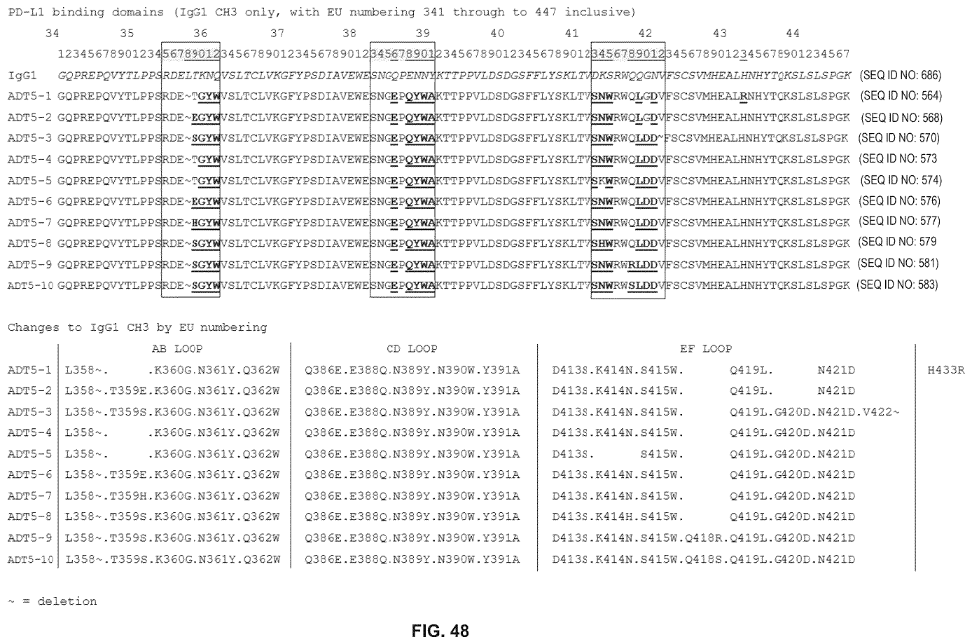

“EU numbering” refers to a numbering convention used for the numbering of amino acid residues in antibodies. Each residue of the antibody is assigned a number to allow comparison of antibodies. Where a variant antibody contains a residue that was not present in the wildtype antibody (e.g. an insertion mutation) decimals are used to indicate the insertion. For example, if a residue was inserted between the residues designated numbers 361 and 362, the new residue would be given the EU number 361.1. EU numbering is an alternative numbering scheme to others such as Kabat, (Kabat et al. Sequences of Proteins of Immunological Interest, Fifth Edition U.S. Department of Health and Human Services, (1991) NIH Publication Number 91-3242). Chothia (Chothia et al. (1989) Nature 342: 877-883) or as summarized by IMGT.org. wherein EU numbering is presented with further cross reference to Edelman, G. M. et al., Proc. Natl. Acad. USA, 63, 78-85 (1969). (10.1073/pnas.63.1.78). FIGS. 28A-28D show the correspondence between EU numbering, IMGT numbering and Kabat numbering for the CH3 domain residues. FIG. 48 shows how the residues of the multispecific antibodies of the disclosure are numbered according to the EU numbering scheme.

A “Fab region” as used herein refers to a portion of an antibody (or constructs that contain said portion) comprising a VH-VL domain pair providing an antigen-binding site (also known as a CDR based antigen binding site). The Fab region may further comprise the CL and CH1 domains. A conventional antibody with two heavy and two light chains thus comprises two Fabs, each Fab comprising a VH-VL domain pair.

A “Fc region” (fragment crystallizable region), as used herein refers to a portion of an antibody (or constructs that contain said portion) comprising the CH2 and CH3 domains. The Fc region is the C-terminal region of an immunoglobulin heavy chain, including wild-type-sequence Fc regions and modified Fc regions. An Fc region is dimeric and thus comprises paired heavy chain constant regions each comprising a CH2 and CH3 domain.

A “fragment” of the antibody (which may also referred to as “antibody fragment”, “immunoglobulin fragment”, “antigen-binding fragment” or “antigen-binding polypeptide”) as used herein refers to a portion of an antibody (or constructs that contain said portion) that specifically binds to the first target, the delta variable 1 (Vδ1) chain of a γδ T cell receptor or the second target, PD-L1 (e.g. a molecule in which one or more immunoglobulin chains is not full length, but which specifically binds to the target). Examples of binding fragments encompassed within the term antibody fragment include:
(i) a Fab fragment (a monovalent fragment consisting of the VL, VH, CL and CH1 domains);
(ii) a F(ab′)2 fragment (a bivalent fragment consisting of two Fab fragments linked by a disulphide bridge at the hinge region);
(iii) a Fd fragment (consisting of the VH and CH1 domains);
(iv) a Fv fragment (consisting of the VL and VH domains of a single arm of an antibody);
(v) a single chain variable fragment, scFv (consisting of VL and VH domains joined, using recombinant methods, by a synthetic linker that enables them to be made as a single protein chain in which the VL and VH regions pair to form monovalent molecules);
(vi) a VH (an immunoglobulin chain variable domain consisting of a VH domain);
(vii) a VL (an immunoglobulin chain variable domain consisting of a VL domain);
(viii) a domain antibody (dAb, consisting of either the VH or VL domain);
(ix) a minibody (consisting of a pair of scFv fragments which are linked via CH3 domains); and
(x) a diabody (consisting of a noncovalent dimer of scFv fragments that consist of a VH domain from one antibody connected by a small peptide linker a VL domain from another antibody)
(xi) a Fc fragment (consisting of the CH2 and CH3 domains)
(xii) a Fcab fragment (consisting of an Fc fragment that has been engineered to contain antigen-binding loops in its CH3 domain)
(xiii) an antigen-binding CH3 domain of an Fcab.

“Human antibody” refers to antibodies having variable and constant regions derived from human germline immunoglobulin sequences. Human subjects administered with said human antibodies do not generate cross-species antibody responses (for example termed HAMA responses—human-anti-mouse antibody) to the primary amino acids contained within said antibodies. Said human antibodies may include amino acid residues not encoded by human germline immunoglobulin sequences (e.g. mutations introduced by random or site-specific mutagenesis or by somatic mutation), for example in the CDRs and in particular CDR3.

However, the term is not intended to include antibodies in which CDR sequences derived from the germline of another mammalian species, such as a mouse, have been grafted onto human framework sequences. Human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g. a mouse) that is transgenic for human immunoglobulin genes or antibodies prepared, expressed, created or isolated by any other means that involves splicing of human immunoglobulin gene sequences to other DNA sequences, may also be referred to as “recombinant human antibodies”.

Substituting at least one amino acid residue in the framework region of a non-human immunoglobulin variable domain with the corresponding residue from a human variable domain is referred to as “humanisation”. Humanisation of a variable domain may reduce immunogenicity in humans.

“Specificity” refers to the number of different types of antigens or antigenic determinants to which a particular antibody or antigen-binding fragment thereof can bind. The specificity of an antibody is the ability of the antibody to recognise a particular antigen as a unique molecular entity and distinguish it from another. An antibody that “specifically binds” to an antigen or an epitope (or is “specific” for an antigen or epitope) is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances.

The antibodies of the present disclosure are multispecific antibodies. A “multispecific antibody” is an antibody that is capable of binding a plurality of different epitopes simultaneously or sequentially. Generally, the epitopes will not be on the same antigen. Hence a multispecific antibody has the capability to selectively bind to epitopes present on different antigens via a plurality of different binding domains. This contrasts with conventional mono-specific antibodies which do not have this capability. Rather, a “monospecific antibody” only has binding specificity for one antigen, although they may have multiple binding sites for that one antigen (e.g. the valency of a full human IgG antibody is 2, and the valency of other antibodies may be higher, but if the antibody only recognises one antigen, it is still classed as a monospecific antibody). Hence, the multispecific antibodies of the disclosure bind multiple different antigens simultaneously and/or sequentially.

In some embodiments of the disclosure, the antibodies are bispecific antibodies. A “bispecific antibody” is an antibody that is capable of binding two different epitopes simultaneously and/or sequentially. Generally, the epitopes will not be on the same antigen. Hence bispecific antibodies have the capability to selectively bind to two different epitopes present on two different antigens via two different binding domains. This contrasts with conventional monospecific antibodies which do not have this capability. Hence, the bispecific antibodies of the disclosure bind two different antigens simultaneously and/or sequentially.

“mAb² antibody or “mAb squared antibody” as used herein, refers to a bispecific antibody format according to which the bispecific antibodies comprise an Fc region that has been engineered to contain antigen-binding loops in its CH3 domain. This modified Fc region is termed an “Fcab”. The mAb² antibody further comprises a Fab region, comprising a VH-VL domain pair providing an antigen-binding site.

The CH3 domain of human IgG1 starts at residue number 341 and ends at residue number 447 (EU numbering). Residue number 447 (EU numbering) is sometimes cleaved during manufacture, so it does not always appear in the final molecule. The CH3 domain of human IgG1 (SEQ ID NO: 563) is comprised of two R-sheets formed by three and four R strands, respectively. Three structural loops connect these R-strands at the C-terminus of the CH3 domain. In antibodies of this disclosure, these loops have been modified to form the PD-L1 binding site. Residues 355 to 362 were defined as the loop connecting strands A and B of the CH3 domain “AB loop”, residues 383 to 391 as the loop connecting strands C and D of the CH3 domain “CD loop” and residues 413 to 422 as the loop connecting strands E and F of the CH3 domain “EF loop”. EU numbering is used (see FIG. 48).

“Affinity”, represented by the equilibrium constant for the dissociation of an antigen with an antigen-binding polypeptide (KD), is a measure of the binding strength between an antigenic determinant and an antigen-binding site on the antibody (or antigen-binding fragment thereof): the lesser the value of the KD, the stronger the binding strength between an antigenic determinant and the antigen-binding polypeptide. Alternatively, the affinity can also be expressed as the affinity constant (KA), which is 1/KD. Affinity can be determined by known methods, depending on the specific antigen of interest. For example. KD may be determined by surface plasmon resonance.

Any KD value less than 10⁻⁶ is considered to indicate binding. Specific binding of an antibody, or antigen-binding fragment thereof, to an antigen or antigenic determinant can be determined in any suitable known manner, including, for example, Scatchard analysis and/or competitive binding assays, such as radioimmunoassays (RIA), enzyme immunoassays (EIA) and sandwich competition assays, equilibrium dialysis, equilibrium binding, gel filtration, ELISA, surface plasmon resonance, or spectroscopy (e.g. using a fluorescence assay) and the different variants thereof known in the art.

“Avidity” is the measure of the strength of binding between an antibody, or antigen-binding fragment thereof, and the pertinent antigen. Avidity is related to both the affinity between an antigenic determinant and its antigen-binding site on the antibody and the number of pertinent binding sites present on the antibody.

“In situ” means in the natural or original place, instead of being moved to another place. For example, an in situ Vδ1+ cell in a patient refers to a vδ1 cell in vivo, as opposed to an in vitro or ex vivo cell.

“Human tissue Vδ1+ cells,” and “haemopoietic and blood Vδ1+ cells” and “tumour infiltrating lymphocyte (TIL) Vδ1+ cells,” are defined as Vδ1+ cells contained in or derived from either human tissue or the haemopoietic blood system or human tumours respectively. All said cell types can be identified by their (i) location or from where they are derived and (ii) their expression of the Vδ1+ TCR.

“Modulating antibodies” are antibodies that confer a measurable change including, but not limited to, a measurable change in cell cycle, and/or in cell number, and/or cell viability, and/or in one or more cell surface markers, and/or in the secretion of one or more secretory molecules (e.g., cytokines, chemokines, leukotrienes, etc.), and/or a function (such as cytotoxicity towards a target cell or diseased cell), upon contacting or binding to a cell expressing the target to which the antibody binds. A method of “modulating” a cell, or population thereof, refers to a method wherein in at least one measurable change in said cell or cells, or secretion therefrom, is triggered to generate one or more “modulated cells”.

An “immune response” is a measurable change in at least one cell, or one cell-type, or one endocrine pathway, or one exocrine pathway, of the immune system (including but not limited to a cell-mediated response, a humoral response, a cytokine response, a chemokine response) upon addition of a modulating antibody.

An “immune cell” is defined as a cell of the immune system including, but not limited to, CD34+ cells, B-Cells, CD45+ (lymphocyte common antigen) cells, Alpha-Beta T-cells, Cytotoxic T-cells, Helper T-cells, Plasma Cells, Neutrophils, Monocytes, Macrophages, Red Blood Cells, Platelets, Dendritic Cells, Phagocytes, Granulocytes, Innate lymphoid cells, Natural Killer (NK) cells and Gamma Delta T-cells. Typically, immune cells are classified with the aid of combinatorial cell surface molecule analysis (e.g., via flow cytometry) to identify or group or cluster to differentiate immune cells into sub-populations. These can be then still further sub-divided with additional analysis. For example, CD45+ lymphocytes can further sub-divided into vδ positive populations and vδ negative populations.

“Model systems” are biological models or biological representations designed to aid in the understanding of how a medicine such as an antibody or antigen-binding fragment thereof may function as a medicament in the amelioration of a sign or symptom of disease. Such models typically include the use of in vitro, ex vivo, and in vivo diseased cells, non-diseased cells, healthy cells, effector cells, and tissues etc., and in which the performance of said medicaments are studied and compared.

“Diseased cells” exhibit a phenotype associated with the progression of a disease such as a cancer, an infection such as a viral infection, or an inflammatory condition or inflammatory disease. For example, a diseased cell may be a tumour cell, an autoimmune tissue cell or a virally infected cell. Accordingly said diseased cells may be defined as tumorous, or virally infected, or inflammatory.

“Healthy cells” refers to normal cells that are not diseased. They may also be referred to as “normal” or “non-diseased” cells. Non-diseased cells include non-cancerous, or non-infected, or non-inflammatory cells. Said cells are often employed alongside relevant diseased cells to determine the diseased cell specificity conferred by a medicament and/or better understand the therapeutic index of a medicament.

“Diseased-cell-specificity” is a measure of how effective an effector cell or population thereof, (such as, for example, a population of Vδ1+ cells) is at distinguishing and killing diseased cells, such as cancer cells, whilst sparing non-diseased or healthy cells. This potential can be measured in model systems and may involve comparing the propensity of an effector cell, or a population of effector cells, to selectively kill or lyse diseased cells versus the potential of said effector cell/s to kill or lyse non-diseased or healthy cells. Said diseased-cell-specificity can inform the potential therapeutic index of a medicament.

“Enhanced diseased-cell specificity” describes a phenotype of an effector cell such as, for example, a Vδ1+ cell, or population thereof, which has been modulated to further increase its capacity to specifically kill diseased cells. This enhancement can be measured in a variety of ways inclusive of fold-change, or percentage increase, in diseased-cell killing specificity or selectivity.

“ADCC” or “antibody-dependent cell-mediated cytotoxicity” describes an immune response to cells coated with antibodies bound to the surface antigens of the cell. It is a cell-mediated process, whereby an immune effector cell (such as a NK cell, for example) recognise cell bound antibodies, triggering degranulation and lysis of the target cell. Typically, this is mediated via Fc-Fcγ interactions. The Fc region of the cell-bound antibody recruits effector cells expressing Fcγ receptors (e.g. NK cells), leading to effector cell degranulation and death of the target cell.

“Fc enabled” refers to an antibody that comprises a functional Fc region (fragment crystallizable region), i.e. a Fc region that has not been disabled by mutation or otherwise. Fc enabled antibodies demonstrate unattenuated Fc function. The Fc enabled antibody may comprise human IGHC heavy chain sequence as listed by IMGT that has not been modified or engineered or constructed to reduce binding to one or more Fc gamma receptors. For example, via IGHC hinge mutation or by construction of an antibody comprising heavy chain constant domains which are chimeric or hybrid for IgG1/IgG2A or IgG1/IgG4 IGHC sequences.

Suitably, the antibody or antigen-binding fragment thereof (i.e. polypeptide) of the disclosure is isolated. An “isolated” polypeptide is one that is removed from its original environment. The term “isolated” may be used to refer to an antibody that is substantially free of other antibodies having different antigenic specificities (e.g. an isolated antibody that specifically binds Vδ1, or a fragment thereof, is substantially free of antibodies that specifically bind antigens other than Vδ1). The term “isolated” may also be used to refer to preparations where the isolated antibody is sufficiently pure to be administered therapeutically when formulated as an active ingredient of a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

Suitably, the polynucleotides used in the present disclosure are isolated. An “isolated” polynucleotide is one that is removed from its original environment. For example, a naturally occurring polynucleotide is isolated if it is separated from some or all of the coexisting materials in the natural system. A polynucleotide is considered to be isolated if, for example, it is cloned into a vector that is not a part of its natural environment or if it is comprised within cDNA.

The antibody or antigen-binding fragment thereof may be a “functionally active variant” which also includes naturally occurring allelic variants, as well as mutants or any other non-naturally occurring variants. As is known in the art, an allelic variant is an alternate form of a (poly)peptide that is characterized as having a substitution, deletion, or addition of one or more amino acids that does essentially not alter the biological function of the polypeptide. By way of non-limiting example, said functionally active variants may still function when the frameworks containing the CDRs are modified, when the CDRs themselves are modified, when said CDRs are grafted to alternate frameworks, or when N- or C-terminal extensions are incorporated. Further, CDR containing binding domains may be paired with differing partner chains such as those shared with another antibody. Upon sharing with so called ‘common’ light or ‘common’ heavy chains, said binding domains may still function. Further, said binding domains may function when multimerized. Further, ‘antibodies or antigen-binding fragments thereof’ may also comprise functional variants wherein the VH or VL or constant domains have been modified away or towards a different canonical sequence (for example as listed at IMGT.org) and which still function.

For the purposes of comparing two closely related polypeptide sequences, the “% sequence identity” between a first polypeptide sequence and a second polypeptide sequence may be calculated using NCBI BLAST v2.0, using standard settings for polypeptide sequences (BLASTP). For the purposes of comparing two closely related polynucleotide sequences, the “% sequence identity” between a first nucleotide sequence and a second nucleotide sequence may be calculated using NCBI BLAST v2.0, using standard settings for nucleotide sequences (BLASTN).

Polypeptide or polynucleotide sequences are said to be the same as or “identical” to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides.

In some embodiments, any specified % sequence identity of a sequence is calculated without the sequences of all 6 CDRs of the antibody. For example, the anti-Vδ1 antibody or antigen-binding fragment thereof may comprise a variable heavy chain region sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% to a specified variable heavy chain region sequence and/or a variable light chain region sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a specified variable light chain region sequence, wherein any amino acid variations occur only in the framework regions of the variable heavy and light chain region sequences. In such embodiments, the anti-Vδ1 antibody or antigen-binding fragment thereof having certain sequence identities retain the complete heavy and light chain CDR1, CDR2 and CDR3 sequences of the corresponding anti-Vδ1 antibody or antigen-binding fragment thereof. In a more specific example, although in no way limiting and only to further illustrate these embodiments of the disclosure, there is provided an anti-Vδ1 antibody or antigen-binding fragment thereof comprising a VH comprising or consisting of an amino acid sequence that is at least 90% identical to the amino acid sequence of SEQ ID NO: 15 and a VL comprising or consisting of an amino acid sequence that is at least 90% identical to the amino acid sequence of SEQ ID NO: 40, wherein any amino acid variations occur only in the framework regions of the variable heavy and light chain region sequences. The antibody of this specific example therefore further comprises a VHCDR1 comprising the amino acid sequence of SEQ ID NO: 51, a VHCDR2 comprising the amino acid sequence of SEQ ID NO: 53, a VHCDR3 comprising the amino acid sequence of SEQ ID NO: 68, a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 95.

Furthermore, multispecific antibodies provided herein may comprise a kappa light chain variable sequence and retain an amino acid residue at position 74 according to the IMGT numbering system that is not serine, for example a non-polar and/or non-human-germline residue, for example they may comprise a leucine residue at this position. For example, although in no way limiting and only to further illustrate these embodiments of the disclosure, there is provided an anti-Vδ1 antibody or antigen-binding fragment thereof comprising a VH comprising or consisting of an amino acid sequence that is at least 90% identical to the amino acid sequence of SEQ ID NO: 15 and a VL comprising or consisting of an amino acid sequence that is at least 90% identical to the amino acid sequence of SEQ ID NO: 40, wherein any amino acid variations occur only in the framework regions of the variable heavy and light chain region sequences, and wherein the antibody comprises a kappa light chain variable sequence comprising an amino acid residue at position 74 according to the IMGT numbering system that is non-human-germline and/or non-polar (for example a leucine residue at this position). The antibody of this specific example further comprises a VHCDR1 comprising the amino acid sequence of SEQ ID NO: 51, a VHCDR2 comprising the amino acid sequence of SEQ ID NO: 53, a VHCDR3 comprising the amino acid sequence of SEQ ID NO: 68, a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 95.

A “difference” between sequences refers to an insertion, deletion or substitution of a single amino acid residue in a position of the second sequence, compared to the first sequence. Two polypeptide sequences can contain one, two or more such amino acid differences. Insertions, deletions or substitutions in a second sequence which is otherwise identical (100% sequence identity) to a first sequence result in reduced % sequence identity. For example, if the identical sequences are 9 amino acid residues long, one substitution in the second sequence results in a sequence identity of 88.9%. If first and second polypeptide sequences are 9 amino acid residues long and share 6 identical residues, the first and second polypeptide sequences share greater than 66% identity (the first and second polypeptide sequences share 66.7% identity).

Alternatively, for the purposes of comparing a first, reference polypeptide sequence to a second, comparison polypeptide sequence, the number of additions, substitutions and/or deletions made to the first sequence to produce the second sequence may be ascertained. An “addition” is the addition of one amino acid residue into the sequence of the first polypeptide (including addition at either terminus of the first polypeptide). A “substitution” is the substitution of one amino acid residue in the sequence of the first polypeptide with one different amino acid residue. Said substitution may be conservative or non-conservative. A “deletion” is the deletion of one amino acid residue from the sequence of the first polypeptide (including deletion at either terminus of the first polypeptide).

Using the three letter and one letter codes the naturally occurring amino acids may be referred to as follows: glycine (G or Gly), alanine (A or Ala), valine (V or Val), leucine (L or Leu), isoleucine (I or lie), proline (P or Pro), phenylalanine (F or Phe), tyrosine (Y or Tyr), tryptophan (W or Trp), lysine (K or Lys), arginine (R or Arg), histidine (H or His), aspartic acid (D or Asp), glutamic acid (E or Glu), asparagine (N or Asn), glutamine (Q or Gln), cysteine (C or Cys), methionine (M or Met), serine (S or Ser) and Threonine (T or Thr). Where a residue may be aspartic acid or asparagine, the symbols Asx or B may be used. Where a residue may be any amino acid the symbol Xaa or X may be used. Where a residue may be glutamic acid or glutamine, the symbols Glx or Z may be used. References to aspartic acid include aspartate, and glutamic acid include glutamate, unless the context specifies otherwise.

As used herein, numbering of polypeptide sequences and definitions of CDRs and FRs are as defined according to the EU and/or IMGT numbering system, as indicated in context. A “corresponding” amino acid residue between a first and second polypeptide sequence is an amino acid residue in a first sequence affinity which shares the same position according to the EU and/or IMGT numbering system, as indicated in context, with an amino acid residue in a second sequence, whilst the amino acid residue in the second sequence may differ in identity from the first. Suitably corresponding residues will share the same number (and letter) if the framework and CDRs are the same length according to EU or IMGT definition. Alignment can be achieved manually or by using, for example, a known computer algorithm for sequence alignment such as NCBI BLAST v2.0 (BLASTP or BLASTN) using standard settings.

References herein to an “epitope” refer to the portion of the target which is specifically bound by the antibody or antigen-binding fragment thereof. Epitopes may also be referred to as “antigenic determinants”. An antibody binds “essentially the same epitope” as another antibody when they both recognize identical or sterically overlapping epitopes. Commonly used methods to determine whether two antibodies bind to identical or overlapping epitopes are competition assays, which can be configured in a number of different formats (e.g. well plates using radioactive or enzyme labels, or flow cytometry on antigen-expressing cells) using either labelled antigen or labelled antibody. An antibody binds “the same epitope” as another antibody when they both recognize identical epitopes (i.e. all contact points between the antigen and the antibody are the same). Reference to an antibody or fragment thereof that specifically binds an epitope of a particular antigen, is equivalent to referring to an antibody or fragment thereof that specifically binds a particular antigen. It is understood that, by definition, an antibody binding to a specific antigen will do so via an epitope. Reference to an antibody or fragment thereof that specifically binds an epitope of a particular antigen therefore does not imply the epitope is known or must be discovered in order to work the disclosure.

Epitopes found on protein targets may be defined as “linear epitopes” or “conformational epitopes”. Linear epitopes are formed by a continuous sequence of amino acids in a protein antigen. Conformational epitopes are formed of amino acids that are discontinuous in the protein sequence, but which are brought together upon folding of the protein into its three-dimensional structure.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g. bacterial vectors having a bacterial origin of replication and episomal mammalian and yeast vectors). Other vectors (e.g. non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g. replication defective retroviruses, adenoviruses and adeno-associated viruses), which serve equivalent functions, and also bacteriophage and phagemid systems. The term “recombinant host cell” (or simply “host cell”), as used herein, is intended to refer to a cell into which a recombinant expression vector has been introduced. Such terms are intended to refer not only to the particular subject cell but to the progeny of such a cell, for example, when said progeny are employed to make a cell line or cell bank which is then optionally stored, provided, sold, transferred, or employed to manufacture an antibody or antigen-binding fragment thereof as described herein.

References to “subject”, “patient” or “individual” refer to a subject, in particular a mammalian subject, to be treated. Mammalian subjects include humans, non-human primates, farm animals (such as cows), sports animals, or pet animals, such as dogs, cats, guinea pigs, rabbits, rats or mice. In some embodiment, the subject is a human. In alternative embodiments, the subject is a non-human mammal, such as a mouse.

The term “sufficient amount” means an amount sufficient to produce a desired effect. The term “therapeutically effective amount” is an amount that is effective to ameliorate a symptom of a disease or disorder. A therapeutically effective amount can be a “prophylactically effective amount” as prophylaxis can be considered therapy.

A disease or disorder is “ameliorated” if the severity of a sign or symptom of the disease or disorder, the frequency with which such a sign or symptom is experienced by a subject, or both, is reduced (compared to an earlier point in time, for example, prior to administration of any antibody).

As used herein, “treating a disease or disorder” means reducing the frequency and/or severity of at least one sign or symptom of the disease or disorder experienced by a subject (compared to an earlier point in time, for example, prior to administration of any antibody).

“Cancer”, as used herein, refers to the abnormal growth or division of cells. Generally, the growth and/or life span of a cancer cell exceeds, and is not coordinated with, that of the normal cells and tissues around it. Cancers may be benign, pre-malignant or malignant.

As used herein, the term “about” when used herein includes up to and including 10% greater and up to and including 10% lower than the value specified, suitably up to and including 5% greater and up to and including 5% lower than the value specified, especially the value specified. The term “between”, includes the values of the specified boundaries.

### Multispecific Antibodies and Antigen-Binding Fragments Thereof

Provided herein are multispecific antibodies capable of specifically binding to the delta variable 1 chain (Vδ1) of a γδ T Cell Receptor (TCR), and also capable of specifically binding to PD-L1. The disclosure relates to the use of said antibodies as medicaments for administration to a subject to be treated.

In one embodiment, the antibody or antigen-binding fragment thereof is an scFv, Fab, Fab′, F(ab′)2, Fv, variable domain (e.g. VH or VL), diabody, minibody or monoclonal antibody. In a further embodiment, the antibody or antigen-binding fragment thereof is an scFv.

Multispecific antibodies of the disclosure can be of any class, e.g. IgG, IgA, IgM, IgE, IgD, or isotypes thereof, and can comprise a kappa or lambda light chain. In one embodiment, the antibody is an IgG antibody, for example, at least one of isotypes, IgG1, IgG2, IgG3 or IgG4. In a further embodiment, the antibody may be in a format, such as an IgG format, that has been modified to confer desired properties, such as having the Fc mutated to reduce effector function, extend half-life, alter ADCC, or improve hinge stability. Such modifications are well known in the art.

In one embodiment, the antibody or antigen-binding fragment thereof is human. Thus, the antibody or antigen-binding fragment thereof may be derived from a human immunoglobulin (Ig) sequence. The CDR, framework and/or constant region of the antibody (or antigen-binding fragment thereof) may be derived from a human Ig sequence, in particular a human IgG sequence. The CDR, framework and/or constant region may be substantially identical for a human Ig sequence, in particular a human IgG sequence. An advantage of using human antibodies is that they are low or non-immunogenic in humans.

An antibody or antigen-binding fragment thereof can also be chimeric, for example a mouse-human antibody chimera.

Alternatively, the antibody or antigen-binding fragment thereof is derived from a non-human species, such as a mouse. Such non-human antibodies can be modified to increase their similarity to antibody variants produced naturally in humans, thus the antibody or antigen-binding fragment thereof can be partially or fully humanised. Therefore, in one embodiment, the antibody or antigen-binding fragment thereof is humanised.

In some embodiments, the multispecific antibodies are Vδ1 agonists and/or PD-L1 antagonists.

A “Vδ1 agonist” activates γδ T cells via the Vδ1 chain of the γδ TCR receptor. For example, binding of a Vδ1 agonist antibody to a Vδ1 chain may stimulate a TCR function, such as cell degranulation, TCR downregulation, cytotoxicity, proliferation, mobilisation, increased survival or resistance to exhaustion, intracellular signalling, cytokine or growth factor secretion, phenotypic change, or a change in gene expression. For example, the binding of the Vδ1 agonist to Vδ1 may stimulate expansion (i.e. proliferation) of the γδ T cell population, preferably the Vδ1+ T cell population. Therefore, in one embodiment, binding of the Vδ1 agonist to Vδ1 downregulates the γδ TCR. In an additional or alternative embodiment, binding of the Vδ1 agonist to Vδ1 activates degranulation of the γδ T cell. In a further additional or alternative embodiment, binding of the Vδ1 agonist to Vδ1 promotes γδ T cell mediated killing.

A “PD-L1 antagonist” blocks the interaction between PD-L1 and it's receptor PD-1. This prevents immune suppression that is caused by PD-L1 on tumour cells interacting with PD-1 on T cells. This removes the immune brake tumours use to evade detection and enhances T-cell mediated anti-tumour responses.

### Summary of Specific Fab Regions Provided Herein

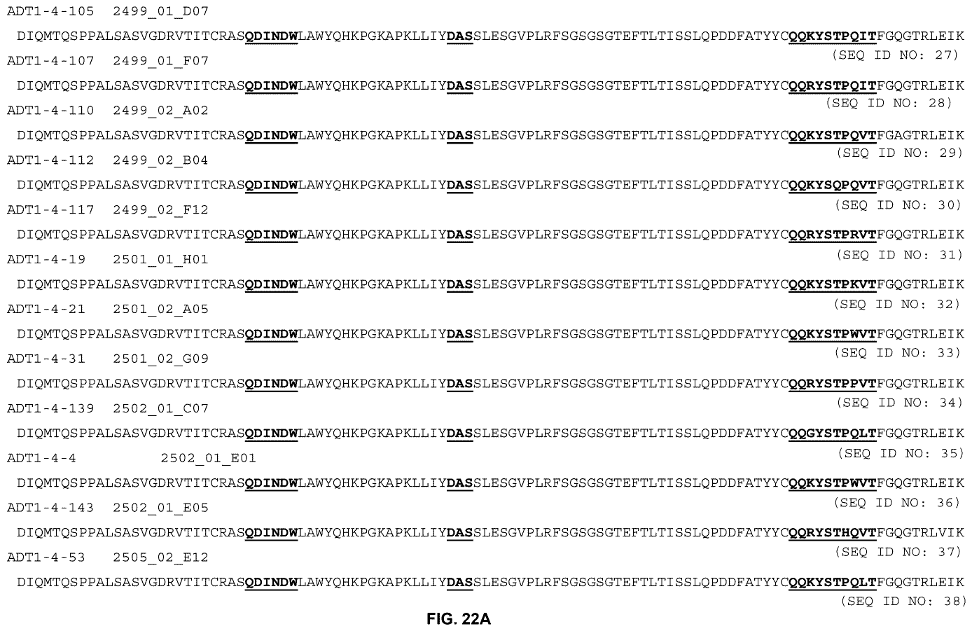

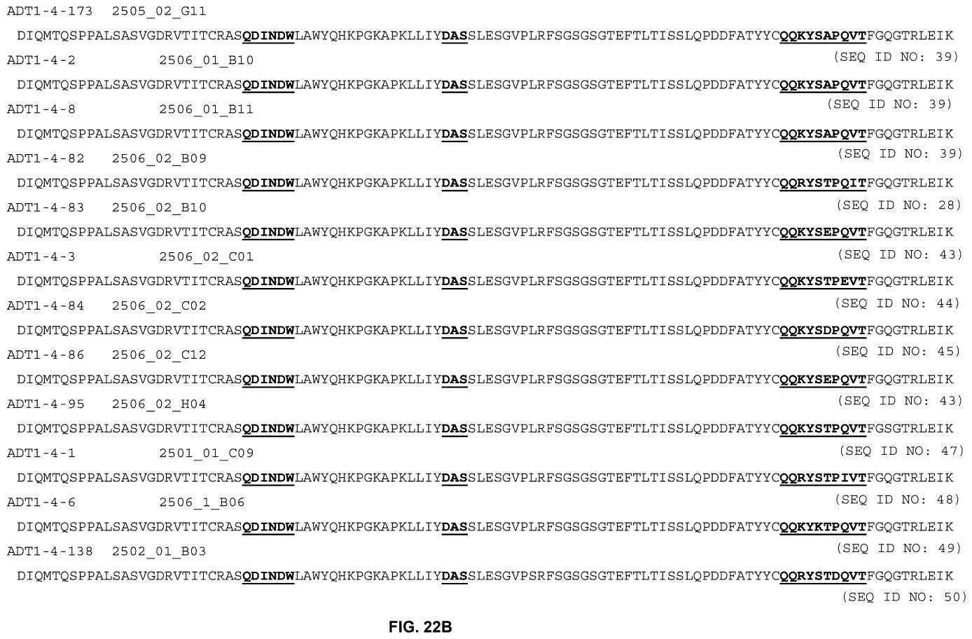

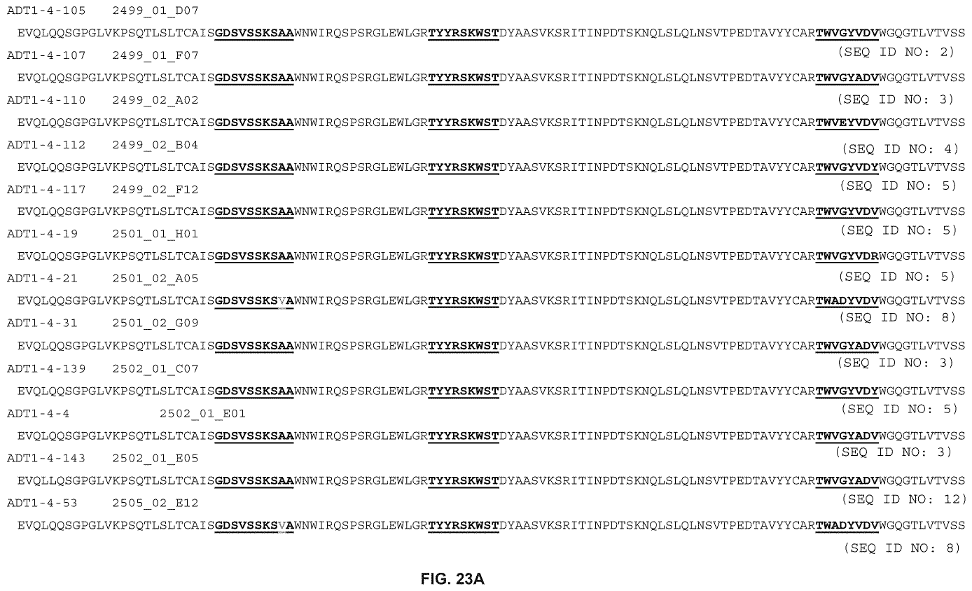

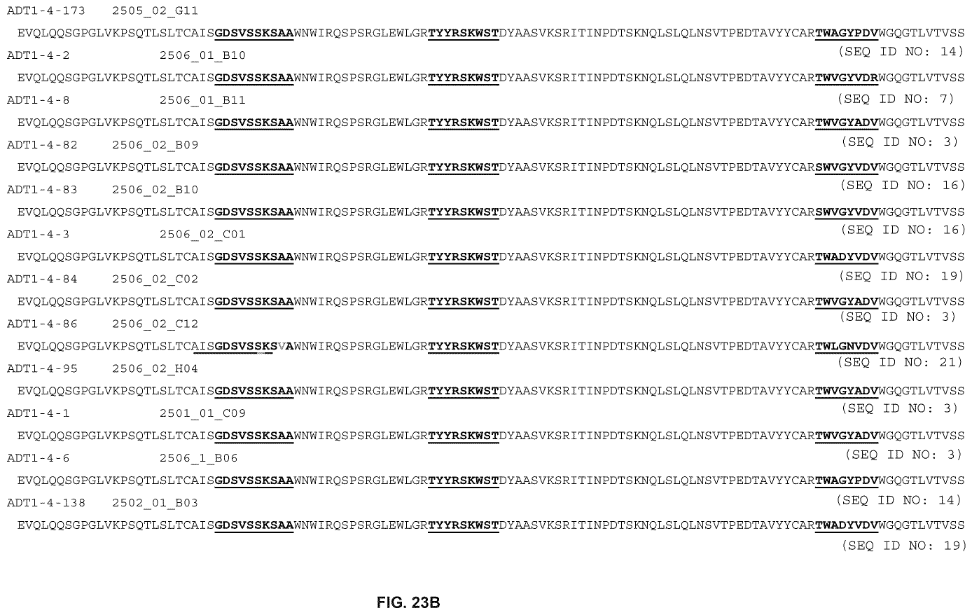

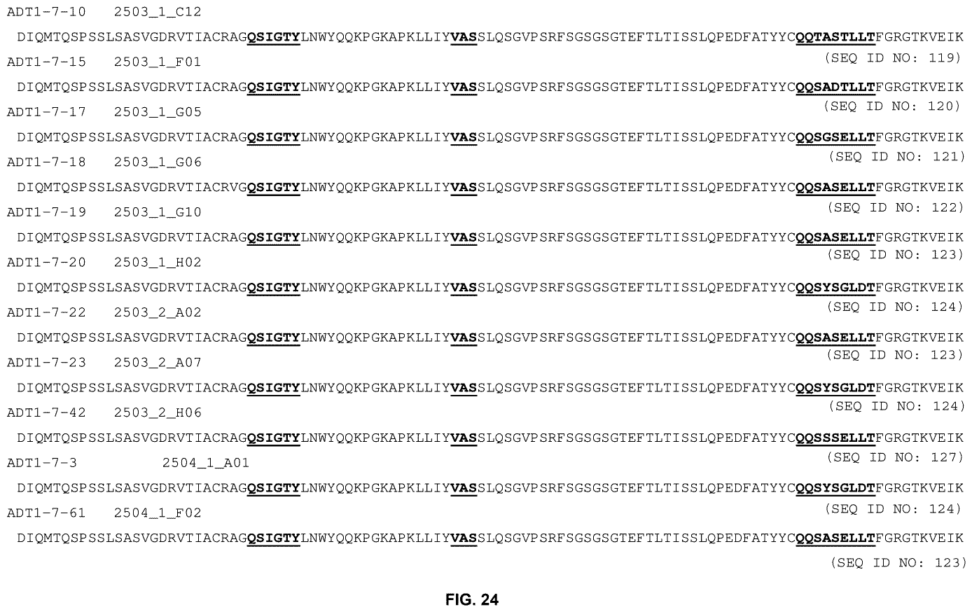

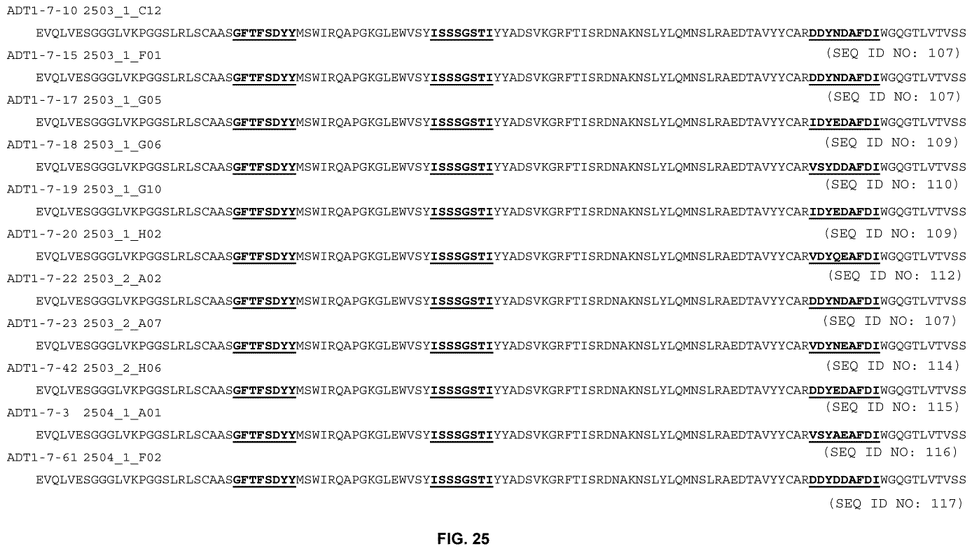

A summary of some of the specific antigen-binding molecules (i.e. antibodies) used in the present disclosure is provided below, with identification of the assigned SEQ ID NO. in the accompanying sequence listing. Antigen-binding variants, derivatives and fragments thereof are also provided as part of the present disclosure. Sequences are provided in the attached sequence listing and the accompanying Figures. In the case of any discrepancy between the sequences in the sequence listing and those in FIGS. 22A to 25, the sequences in the Figures should prevail.

TABLE 1
Summary of Fab regions derived from ADT1-4
(heavy chain) and the related SEQ ID NOs

| Antibody | VH | HFR1 | VHCDR1 | HFR2 | VHCDR2 | HFR3 | VHCDR3 | HFR4 |
|---|---|---|---|---|---|---|---|---|
| Parental | 1 | 170 | 51 | 172 | 53 | 173 | 54 | 174 |
| ADT1-4 | | | | | | | | |
| ADT1-4-105 | 2 | 170 | 51 | 172 | 53 | 173 | 55 | 174 |
| ADT1-4-107 | 3 | 170 | 51 | 172 | 53 | 173 | 56 | 174 |
| ADT1-4-110 | 4 | 170 | 51 | 172 | 53 | 173 | 57 | 174 |
| ADT1-4-112 | 5 | 170 | 51 | 172 | 53 | 173 | 58 | 174 |
| ADT1-4-117 | 6 | 170 | 51 | 172 | 53 | 173 | 59 | 174 |
| ADT1-4-19 | 7 | 170 | 51 | 172 | 53 | 173 | 60 | 174 |
| ADT1-4-21 | 8 | 170 | 52 | 172 | 53 | 173 | 61 | 174 |
| ADT1-4-31 | 9 | 170 | 51 | 172 | 53 | 173 | 62 | 174 |
| ADT1-4-139 | 10 | 170 | 51 | 172 | 53 | 173 | 63 | 174 |
| ADT1-4-4 | 11 | 170 | 51 | 172 | 53 | 173 | 64 | 174 |
| ADT1-4-143 | 12 | 171 | 51 | 172 | 53 | 173 | 65 | 174 |
| ADT1-4-53 | 13 | 170 | 52 | 172 | 53 | 173 | 66 | 174 |
| ADT1-4-173 | 14 | 170 | 51 | 172 | 53 | 173 | 67 | 174 |
| ADT1-4-2 | 15 | 170 | 51 | 172 | 53 | 173 | 68 | 174 |
| ADT1-4-8 | 16 | 170 | 51 | 172 | 53 | 173 | 69 | 174 |
| ADT1-4-82 | 17 | 170 | 51 | 172 | 53 | 173 | 70 | 174 |
| ADT1-4-83 | 18 | 170 | 51 | 172 | 53 | 173 | 71 | 174 |
| ADT1-4-3 | 19 | 170 | 51 | 172 | 53 | 173 | 72 | 174 |
| ADT1-4-84 | 20 | 170 | 51 | 172 | 53 | 173 | 73 | 174 |
| ADT1-4-86 | 21 | 170 | 52 | 172 | 53 | 173 | 74 | 174 |
| ADT1-4-95 | 22 | 170 | 51 | 172 | 53 | 173 | 75 | 174 |
| ADT1-4-1 | 23 | 170 | 51 | 172 | 53 | 173 | 76 | 174 |
| ADT1-4-6 | 24 | 170 | 51 | 172 | 53 | 173 | 77 | 174 |
| ADT1-4-138 | 25 | 170 | 51 | 172 | 53 | 173 | 78 | 174 |

TABLE 2
Summary of Fab regions derived from ADT1-4
(light chain) and the related SEQ ID NOs

| Antibody | VL | LFR1 | VLCDR1 | LFR2 | VLCDR2 | LFR3 | VLCDR3 | LFR4 |
|---|---|---|---|---|---|---|---|---|
| Parental | 26 | 175 | 79 | 176 | 80 | 178 | 81 | 179 |
| ADT1-4 | | | | | | | | |
| ADT1-4-105 | 27 | 175 | 79 | 176 | 80 | 177 | 82 | 179 |
| ADT1-4-107 | 28 | 175 | 79 | 176 | 80 | 177 | 83 | 179 |
| ADT1-4-110 | 29 | 175 | 79 | 176 | 80 | 177 | 84 | 180 |
| ADT1-4-112 | 30 | 175 | 79 | 176 | 80 | 177 | 85 | 179 |
| ADT1-4-117 | 31 | 175 | 79 | 176 | 80 | 177 | 86 | 179 |
| ADT1-4-19 | 32 | 175 | 79 | 176 | 80 | 177 | 87 | 179 |
| ADT1-4-21 | 33 | 175 | 79 | 176 | 80 | 177 | 88 | 179 |
| ADT1-4-31 | 34 | 175 | 79 | 176 | 80 | 177 | 89 | 179 |
| ADT1-4-139 | 35 | 175 | 79 | 176 | 80 | 177 | 90 | 179 |
| ADT1-4-4 | 36 | 175 | 79 | 176 | 80 | 177 | 91 | 179 |
| ADT1-4-143 | 37 | 175 | 79 | 176 | 80 | 177 | 92 | 181 |
| ADT1-4-53 | 38 | 175 | 79 | 176 | 80 | 177 | 93 | 179 |
| ADT1-4-173 | 39 | 175 | 79 | 176 | 80 | 177 | 94 | 179 |
| ADT1-4-2 | 40 | 175 | 79 | 176 | 80 | 177 | 95 | 179 |
| ADT1-4-8 | 41 | 175 | 79 | 176 | 80 | 177 | 96 | 179 |
| ADT1-4-82 | 42 | 175 | 79 | 176 | 80 | 177 | 97 | 179 |
| ADT1-4-83 | 43 | 175 | 79 | 176 | 80 | 177 | 98 | 179 |
| ADT1-4-3 | 44 | 175 | 79 | 176 | 80 | 177 | 99 | 179 |
| ADT1-4-84 | 45 | 175 | 79 | 176 | 80 | 177 | 100 | 179 |
| ADT1-4-86 | 46 | 175 | 79 | 176 | 80 | 177 | 101 | 179 |
| ADT1-4-95 | 47 | 175 | 79 | 176 | 80 | 177 | 102 | 182 |
| ADT1-4-1 | 48 | 175 | 79 | 176 | 80 | 177 | 103 | 179 |
| ADT1-4-6 | 49 | 175 | 79 | 176 | 80 | 177 | 104 | 179 |
| ADT1-4-138 | 50 | 175 | 79 | 176 | 80 | 178 | 105 | 179 |

TABLE 3
Summary of Fab regions derived from ADT1-7
(heavy chain) and the related SEQ ID NOs

| Antibody | VH | HFR1 | VHCDR1 | HFR2 | VHCDR2 | HFR3 | VHCDR3 | HFR4 |
|---|---|---|---|---|---|---|---|---|
| Parental | 106 | 189 | 130 | 190 | 131 | 191 | 132 | 192 |
| ADT1-4 | | | | | | | | |
| ADT1-7-10 | 107 | 189 | 130 | 190 | 131 | 191 | 133 | 192 |
| ADT1-7-15 | 108 | 189 | 130 | 190 | 131 | 191 | 134 | 192 |
| ADT1-7-17 | 109 | 189 | 130 | 190 | 131 | 191 | 135 | 192 |
| ADT1-7-18 | 110 | 189 | 130 | 190 | 131 | 191 | 136 | 192 |
| ADT1-7-19 | 111 | 189 | 130 | 190 | 131 | 191 | 137 | 192 |
| ADT1-7-20 | 112 | 189 | 130 | 190 | 131 | 191 | 138 | 192 |
| ADT1-7-22 | 113 | 189 | 130 | 190 | 131 | 191 | 139 | 192 |
| ADT1-7-23 | 114 | 189 | 130 | 190 | 131 | 191 | 140 | 192 |
| ADT1-7-42 | 115 | 189 | 130 | 190 | 131 | 191 | 141 | 192 |
| ADT1-7-3 | 116 | 189 | 130 | 190 | 131 | 191 | 142 | 192 |
| ADT1-7-61 | 117 | 189 | 130 | 190 | 131 | 191 | 143 | 192 |

TABLE 4
Summary of Fab regions derived from ADT1-7
(light chain) and the related SEQ ID NOs

| Antibody | VL | LFR1 | VLCDR1 | LFR2 | VLCDR2 | LFR3 | VLCDR3 | LFR4 |
|---|---|---|---|---|---|---|---|---|
| Parental | 118 | 193 | 144 | 195 | 145 | 196 | 146 | 197 |
| ADT1-7 | | | | | | | | |
| ADT1-7-10 | 119 | 193 | 144 | 195 | 145 | 196 | 147 | 197 |
| ADT1-7-15 | 120 | 193 | 144 | 195 | 145 | 196 | 148 | 197 |
| ADT1-7-17 | 121 | 193 | 144 | 195 | 145 | 196 | 149 | 197 |
| ADT1-7-18 | 122 | 194 | 144 | 195 | 145 | 196 | 150 | 197 |
| ADT1-7-19 | 123 | 193 | 144 | 195 | 145 | 196 | 151 | 197 |
| ADT1-7-20 | 124 | 193 | 144 | 195 | 145 | 196 | 152 | 197 |
| ADT1-7-22 | 125 | 193 | 144 | 195 | 145 | 196 | 153 | 197 |
| ADT1-7-23 | 126 | 193 | 144 | 195 | 145 | 196 | 154 | 197 |
| ADT1-7-42 | 127 | 193 | 144 | 195 | 145 | 196 | 155 | 197 |
| ADT1-7-3 | 128 | 193 | 144 | 195 | 145 | 196 | 156 | 197 |
| ADT1-7-61 | 129 | 193 | 144 | 195 | 145 | 196 | 157 | 197 |

TABLE 5
Summary of other parental Fab regions
and the related SEQ ID NOs

| Parental | | |
|---|---|---|
| Antibody | VH | VL |
| C08 | 273 | 282 |
| B07 | 274 | 283 |
| C05 | 275 | 284 |
| E04 | 276 | 285 |
| F07 | 277 | 286 |
| G06 | 278 | 287 |
| G09 | 279 | 288 |
| B09 | 280 | 289 |
| G10 | 281 | 290 |
| E01 | 312 | 313 |

### ADT1-4- and ADT1-7-Derived Fab Regions

The present disclosure provides multispecific antibodies wherein the Fab regions are derived from parental antibody ADT1-4 (having a variable heavy region sequence according to SEQ ID NO: 1 and a variable light region sequence according to SEQ ID NO: 26), and antibodies derived from parental antibody ADT1-7 (having a variable heavy region sequence according to SEQ ID NO: 106 and a variable light region sequence according to SEQ ID No: 118). ADT1-4 is also referred to herein as G04, and ADT1-4 and G04 are used interchangeably. ADT1-7 is also referred to herein as E07, and ADT1-7 and E07 are used interchangeably.

In some embodiments, the disclosure provides multispecific antibodies, the anti-TCR delta variable 1 (anti-Vδ1) binding Fab region comprising:
a heavy chain variable region comprising or consisting of a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 55 to 78 and 133 to 143; and/or a light chain variable region comprising a VLCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 82 to 105 and 147 to 157. Certain amino acid substitutions may be made to provide one or more variant antibodies as described herein.

In some embodiments, the disclosure provides multispecific antibodies, the anti-TCR delta variable 1 (anti-Vδ1) binding Fab region comprising:
a heavy chain variable region comprising:
   a VHCDR1 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NOs: 51, 52 and 130;
   a VHCDR2 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NO: 53 and 131; and
   a VHCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NOs: 55 to 78 and 133 to 143; and
a light chain variable region comprising:
   a VLCDR1 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NO: 79 and 144;
   a VLCDR2 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NO: 80 and 145; and
   a VLCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NOs: 82 to 105 and 147 to 157.

Certain amino acid substitutions may be made to provide one or more variant Fab regions as described herein.

In some embodiments, the disclosure provides multispecific antibodies, the anti-TCR delta variable 1 (anti-Vδ1) binding Fab region comprising:
a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 2 to 25 and 107 to 117; and/or
a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 27 to 50 and 119 to 129.

### ADT1-4-Derived Fab Regions

The present disclosure provides multispecific antibodies comprising Fab regions derived from parental antibody ADT1-4 (having a variable heavy region sequence according to SEQ ID NO: 1 and a variable light region sequence according to SEQ ID NO: 26), for example as set out in the following. ADT1-4 is also referred to herein as G04, and ADT1-4 and G04 are used interchangeably.

Antibodies Comprising Particular CDR Sequences Derived from ADT1-4

The multispecific antibodies provided herein include the following Fab regions having particular sequences derived from ADT1-4.

For example, in some embodiments, there is provided multispecific antibodies, the anti-TCR delta variable 1 (anti-Vδ1) binding Fab region comprising a heavy chain variable region comprising a VHCDR3 comprising or consisting of an amino acid sequence selected from the group consisting of SEQ ID NOs: 55 to 78; and/or a light chain variable region comprising a VLCDR3 comprising or consisting of an amino acid sequence selected from the group consisting of SEQ ID NOs: 82 to 105. Certain amino acid substitutions may be made to provide one or more variant antibodies as described herein.

In some embodiments, there are provided multispecific antibodies, the anti-TCR delta variable 1 (anti-Vδ1) binding Fab region comprising a heavy chain variable region comprising a VHCDR3 comprising or consisting of an amino acid sequence selected from the group consisting of SEQ ID NOs: 55 to 77; and/or a light chain variable region comprising a VLCDR3 comprising or consisting of an amino acid sequence selected from the group consisting of SEQ ID NOs: 82 to 104. Certain amino acid substitutions may be made to provide one or more variant antibodies as described herein.

In some embodiments, there are multispecific antibodies, the anti-TCR delta variable 1 (anti-Vδ1) binding Fab region comprising a heavy chain variable region comprising a VHCDR3 comprising or consisting of an amino acid sequence selected from the group consisting of SEQ ID NOs: 58, 60, 61, 62, 65, 66, 68, 74, 76 and 77; and/or a light chain variable region comprising a VLCDR3 comprising or consisting of an amino acid sequence selected from the group consisting of SEQ ID NOs: 85, 87, 88, 89, 92, 93, 95, 101, 103 and 104. Certain amino acid substitutions may be made to provide one or more variant antibodies as described herein.

There are also provided multispecific antibodies, the anti-TCR delta variable 1 (anti-Vδ1) binding Fab region comprising:
a heavy chain variable region comprising:
   a VHCDR1 comprising or consisting of an amino acid sequence selected from the group consisting of: SEQ ID NOs: 51 and 52;
   a VHCDR2 comprising or consisting of the amino acid sequence of SEQ ID NO: 53; and
   a VHCDR3 comprising or consisting of an amino acid sequence selected from the group consisting of: SEQ ID NOs: 55 to 78; and
a light chain variable region comprising:
   a VLCDR1 comprising or consisting of the amino acid sequence of SEQ ID NO: 79;
   a VLCDR2 comprising or consisting of the amino acid sequence of SEQ ID NO: 80; and
   a VLCDR3 comprising or consisting of an amino acid sequence selected from the group consisting of: SEQ ID NOs: 82 to 105.

Certain amino acid substitutions may be made to provide one or more variant antibodies as described herein.

There are also provided multispecific antibodies, the anti-TCR delta variable 1 (anti-Vδ1) binding Fab region comprising:
a heavy chain variable region comprising:
   a VHCDR1 comprising or consisting of an amino acid sequence selected from the group consisting of: SEQ ID NOs: 51 and 52;
   a VHCDR2 comprising or consisting of the amino acid sequence of SEQ ID NO: 53; and
   a VHCDR3 comprising or consisting of an amino acid sequence selected from the group consisting of: SEQ ID NOs: 55 to 77; and
a light chain variable region comprising:
   a VLCDR1 comprising or consisting of the amino acid sequence of SEQ ID NO: 79;
   a VLCDR2 comprising or consisting of the amino acid sequence of SEQ ID NO: 80; and
   a VLCDR3 comprising or consisting of an amino acid sequence selected from the group consisting of: SEQ ID NOs: 82 to 104.

Certain amino acid substitutions may be made to provide one or more variant antibodies as described herein.

There are also provided multispecific antibodies, the anti-TCR delta variable 1 (anti-Vδ1) binding Fab region comprising:
a heavy chain variable region comprising:
   a VHCDR1 comprising or consisting of an amino acid sequence selected from the group consisting of: SEQ ID NOs: 51 and 52;
   a VHCDR2 comprising or consisting of the amino acid sequence of SEQ ID NO: 53; and
   a VHCDR3 comprising or consisting of an amino acid sequence selected from the group consisting of: SEQ ID NOs: 58, 60, 61, 62, 65, 66, 68, 74, 76 and 77; and
a light chain variable region comprising:
   a VLCDR1 comprising or consisting of the amino acid sequence of SEQ ID NO: 79;
   a VLCDR2 comprising or consisting of the amino acid sequence of SEQ ID NO: 80; and
   a VLCDR3 comprising or consisting of an amino acid sequence selected from the group consisting of: SEQ ID NOs: 85, 87, 88, 89, 92, 93, 95, 101, 103 and 104.

Certain amino acid substitutions may be made to provide one or more variant antibodies as described herein.

There are also provided multispecific antibodies, the anti-TCR delta variable 1 (anti-Vδ1) binding Fab region comprising
a) a VHCDR1, a VHCDR2 and a VHCDR3 comprising or consisting of the amino acid sequences of SEQ ID NO: 51, 53 and 68, respectively, and a VLCDR1, a VLCDR2 and a VLCDR3 comprising or consisting of the amino acid sequences of SEQ ID NOs: 79, 80 and 95, respectively;
b) a VHCDR1, a VHCDR2 and a VHCDR3 comprising or consisting of the amino acid sequences of SEQ ID NO: 51, 53 and 55, respectively, and a VLCDR1, a VLCDR2 and a VLCDR3 comprising or consisting of the amino acid sequences of SEQ ID NOs: 79, 80 and 82, respectively;
c) a VHCDR1, a VHCDR2 and a VHCDR3 comprising or consisting of the amino acid sequences of SEQ ID NO: 51, 53 and 56, respectively, and a VLCDR1, a VLCDR2 and a VLCDR3 comprising or consisting of the amino acid sequences of SEQ ID NOs: 79, 80 and 83, respectively;
d) a VHCDR1, a VHCDR2 and a VHCDR3 comprising or consisting of the amino acid sequences of SEQ ID NO: 51, 53 and 57, respectively, and a VLCDR1, a VLCDR2 and a VLCDR3 comprising or consisting of the amino acid sequences of SEQ ID NOs: 79, 80 and 84, respectively;
e) a VHCDR1, a VHCDR2 and a VHCDR3 comprising or consisting of the amino acid sequences of SEQ ID NO: 51, 53 and 58, respectively, and a VLCDR1, a VLCDR2 and a VLCDR3 comprising or consisting of the amino acid sequences of SEQ ID NOs: 79, 80 and 85, respectively;
f) a VHCDR1, a VHCDR2 and a VHCDR3 comprising or consisting of the amino acid sequences of SEQ ID NO: 51, 53 and 59, respectively, and a VLCDR1, a VLCDR2 and a VLCDR3 comprising or consisting of the amino acid sequences of SEQ ID NOs: 79, 80 and 86, respectively;
g) a VHCDR1, a VHCDR2 and a VHCDR3 comprising or consisting of the amino acid sequences of SEQ ID NO: 51, 53 and 60, respectively, and a VLCDR1, a VLCDR2 and a VLCDR3 comprising or consisting of the amino acid sequences of SEQ ID NOs: 79, 80 and 87, respectively;
h) a VHCDR1, a VHCDR2 and a VHCDR3 comprising or consisting of the amino acid sequences of SEQ ID NO: 52, 53 and 61, respectively, and a VLCDR1, a VLCDR2 and a VLCDR3 comprising or consisting of the amino acid sequences of SEQ ID NOs: 79, 80 and 88, respectively;
i) a VHCDR1, a VHCDR2 and a VHCDR3 comprising or consisting of the amino acid sequences of SEQ ID NO: 51, 53 and 62, respectively, and a VLCDR1, a VLCDR2 and a VLCDR3 comprising or consisting of the amino acid sequences of SEQ ID NOs: 79, 80 and 89, respectively;
j) a VHCDR1, a VHCDR2 and a VHCDR3 comprising or consisting of the amino acid sequences of SEQ ID NO: 51, 53 and 63, respectively, and a VLCDR1, a VLCDR2 and a VLCDR3 comprising or consisting of the amino acid sequences of SEQ ID NOs: 79, 80 and 90, respectively;
k) a VHCDR1, a VHCDR2 and a VHCDR3 comprising or consisting of the amino acid sequences of SEQ ID NO: 51, 53 and 64, respectively, and a VLCDR1, a VLCDR2 and a VLCDR3 comprising or consisting of the amino acid sequences of SEQ ID NOs: 79, 80 and 91, respectively;
l) a VHCDR1, a VHCDR2 and a VHCDR3 comprising or consisting of the amino acid sequences of SEQ ID NO: 51, 53 and 65, respectively, and a VLCDR1, a VLCDR2 and a VLCDR3 comprising or consisting of the amino acid sequences of SEQ ID NOs: 79, 80 and 92, respectively;
m) a VHCDR1, a VHCDR2 and a VHCDR3 comprising or consisting of the amino acid sequences of SEQ ID NO: 52, 53 and 66, respectively, and a VLCDR1, a VLCDR2 and a VLCDR3 comprising or consisting of the amino acid sequences of SEQ ID NOs: 79, 80 and 93, respectively;
n) a VHCDR1, a VHCDR2 and a VHCDR3 comprising or consisting of the amino acid sequences of SEQ ID NO: 51, 53 and 67, respectively, and a VLCDR1, a VLCDR2 and a VLCDR3 comprising or consisting of the amino acid sequences of SEQ ID NOs: 79, 80 and 94, respectively;
o) a VHCDR1, a VHCDR2 and a VHCDR3 comprising or consisting of the amino acid sequences of SEQ ID NO: 51, 53 and 68, respectively, and a VLCDR1, a VLCDR2 and a VLCDR3 comprising or consisting of the amino acid sequences of SEQ ID NOs: 79, 80 and 95, respectively;
p) a VHCDR1, a VHCDR2 and a VHCDR3 comprising or consisting of the amino acid sequences of SEQ ID NO: 51, 53 and 69, respectively, and a VLCDR1, a VLCDR2 and a VLCDR3 comprising or consisting of the amino acid sequences of SEQ ID NOs: 79, 80 and 96, respectively;
q) a VHCDR1, a VHCDR2 and a VHCDR3 comprising or consisting of the amino acid sequences of SEQ ID NO: 51, 53 and 70, respectively, and a VLCDR1, a VLCDR2 and a VLCDR3 comprising or consisting of the amino acid sequences of SEQ ID NOs: 79, 80 and 97, respectively;
r) a VHCDR1, a VHCDR2 and a VHCDR3 comprising or consisting of the amino acid sequences of SEQ ID NO: 51, 53 and 71, respectively, and a VLCDR1, a VLCDR2 and a VLCDR3 comprising or consisting of the amino acid sequences of SEQ ID NOs: 79, 80 and 98, respectively;
s) a VHCDR1, a VHCDR2 and a VHCDR3 comprising or consisting of the amino acid sequences of SEQ ID NO: 51, 53 and 72, respectively, and a VLCDR1, a VLCDR2 and a VLCDR3 comprising or consisting of the amino acid sequences of SEQ ID NOs: 79, 80 and 99, respectively;
t) a VHCDR1, a VHCDR2 and a VHCDR3 comprising or consisting of the amino acid sequences of SEQ ID NO: 51, 53 and 73, respectively, and a VLCDR1, a VLCDR2 and a VLCDR3 comprising or consisting of the amino acid sequences of SEQ ID NOs: 79, 80 and 100, respectively;
u) a VHCDR1, a VHCDR2 and a VHCDR3 comprising or consisting of the amino acid sequences of SEQ ID NO: 52, 53 and 74, respectively, and a VLCDR1, a VLCDR2 and a VLCDR3 comprising or consisting of the amino acid sequences of SEQ ID NOs: 79, 80 and 101, respectively;
v) a VHCDR1, a VHCDR2 and a VHCDR3 comprising or consisting of the amino acid sequences of SEQ ID NO: 51, 53 and 75, respectively, and a VLCDR1, a VLCDR2 and a VLCDR3 comprising or consisting of the amino acid sequences of SEQ ID NOs: 79, 80 and 102, respectively;
w) a VHCDR1, a VHCDR2 and a VHCDR3 comprising or consisting of the amino acid sequences of SEQ ID NO: 51, 53 and 76, respectively, and a VLCDR1, a VLCDR2 and a VLCDR3 comprising or consisting of the amino acid sequences of SEQ ID NOs: 79, 80 and 103, respectively;
x) a VHCDR1, a VHCDR2 and a VHCDR3 comprising or consisting of the amino acid sequences of SEQ ID NO: 51, 53 and 77, respectively, and a VLCDR1, a VLCDR2 and a VLCDR3 comprising or consisting of the amino acid sequences of SEQ ID NOs: 79, 80 and 104, respectively; or;
y) a VHCDR1, a VHCDR2 and a VHCDR3 comprising or consisting of the amino acid sequences of SEQ ID NO: 51, 53 and 78, respectively, and a VLCDR1, a VLCDR2 and a VLCDR3 comprising or consisting of the amino acid sequences of SEQ ID NOs: 79, 80 and 105, respectively.

The multispecific antibodies may comprise a kappa light chain variable sequence (or comprise a variable light chain that is derived from a kappa light chain variable sequence), wherein the residue at position 74 of the kappa light chain variable sequence according to the IMGT numbering system is not serine, for example a non-human-germline residue and/or a non-polar residue at position 74, for example the residue at position 74 is a leucine residue.

Further embodiments are provided below.

### ADT1-4-105 and Fragments and Variants Thereof

Certain embodiments relate to the antibody ADT1-4-105 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 55 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 82. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 55 and/or a light chain variable region comprising a VLCDR3 comprising an amino acid sequence having at least 90% identity to the amino acid sequence of SEQ ID NO: 82. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 55 and/or a light chain variable region comprising a VLCDR3 comprising the amino acid sequence of SEQ ID NO: 82.

Amino acid substitutions may be made to provide variant antibodies with Fab regions derived from ADT1-4-105, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 55, optionally comprising 1 or 2 amino acid substitutions, and/or a light chain variable region comprising a VLCDR3 comprising the amino acid sequence of SEQ ID NO: 82, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 55; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 82.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 55; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 82.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 55; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 82.

Amino acid substitutions may be made to provide variant antibodies with Fab regions derived from ADT1-4-105, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 55 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 82 optionally comprising 1 or 2 amino acid substitutions. The amino acid substitutions may be conservative amino acid substitutions.

The antibodies may alternatively consist of the specified sequences (with or without amino acid substitutions).

### ADT1-4-107 and Fragments and Variants Thereof

Certain embodiments relate to the antibody ADT1-4-107 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 56 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO:83. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 56 and/or a light chain variable region comprising a VLCDR3 comprising an amino acid sequence having at least 90% identity to the amino acid sequence of SEQ ID NO: 83. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 56 and/or a light chain variable region comprising a VLCDR3 comprising the amino acid sequence of SEQ ID NO: 83.

Amino acid substitutions may be made to provide variant antibodies comprising Fab regions derived from ADT1-4-107, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 56, optionally comprising 1 or 2 amino acid substitutions, and/or a light chain variable region comprising a VLCDR3 comprising the amino acid sequence of SEQ ID NO: 83, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 56; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 83.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 56; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 83.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 56; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 83.

Amino acid substitutions may be made to provide variant antibodies comprising Fab regions derived from ADT1-4-107, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 56 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 83 optionally comprising 1 or 2 amino acid substitutions. The amino acid substitutions may be conservative amino acid substitutions.

The antibodies may alternatively consist of the specified sequences (with or without amino acid substitutions).

### ADT1-4-110 and Fragments and Variants Thereof

Certain embodiments relate to the antibody ADT1-4-110 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 57 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 84. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 57 and/or a light chain variable region comprising a VLCDR3 comprising an amino acid sequence having at least 90% identity to the amino acid sequence of SEQ ID NO: 84. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 57 and/or a light chain variable region comprising a VLCDR3 comprising the amino acid sequence of SEQ ID NO: 84.

Amino acid substitutions may be made to provide variant antibodies comprising Fab regions derived from ADT1-4-110, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 57, optionally comprising 1 or 2 amino acid substitutions, and/or a light chain variable region comprising a VLCDR3 comprising the amino acid sequence of SEQ ID NO: 84, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 57; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 84.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 57; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 84.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 57; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 84.

Amino acid substitutions may be made to provide variant antibodies comprising Fab regions derived from ADT1-4-110, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 57 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 84 optionally comprising 1 or 2 amino acid substitutions. The amino acid substitutions may be conservative amino acid substitutions.

The antibodies may alternatively consist of the specified sequences (with or without amino acid substitutions).

### ADT1-4-112 and Fragments and Variants Thereof

Certain embodiments relate to the antibody ADT1-4-112 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 58 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 85. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 58 and/or a light chain variable region comprising a VLCDR3 comprising an amino acid sequence having at least 90% identity to the amino acid sequence of SEQ ID NO: 85. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 58 and/or a light chain variable region comprising a VLCDR3 comprising the amino acid sequence of SEQ ID NO: 85.

Amino acid substitutions may be made to provide variant antibodies having Fab regions derived from ADT1-4-112, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 58, optionally comprising 1 or 2 amino acid substitutions, and/or a light chain variable region comprising a VLCDR3 comprising the amino acid sequence of SEQ ID NO: 85, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 58; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 85.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 58; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 85.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 58; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 85.

Amino acid substitutions may be made to provide variant antibodies with Fab regions derived from ADT1-4-112, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 58 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 85 optionally comprising 1 or 2 amino acid substitutions. The amino acid substitutions may be conservative amino acid substitutions.

The antibodies may alternatively consist of the specified sequences (with or without amino acid substitutions).

### ADT1-4-117 and Fragments and Variants Thereof

Certain embodiments relate to the antibody ADT1-4-117 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 59 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 86. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 59 and/or a light chain variable region comprising a VLCDR3 comprising an amino acid sequence having at least 90% identity to the amino acid sequence of SEQ ID NO: 86. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 59 and/or a light chain variable region comprising a VLCDR3 comprising the amino acid sequence of SEQ ID NO: 86.

Amino acid substitutions may be made to provide variant antibodies with Fab regions derived from ADT1-4-117, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 59, optionally comprising 1 or 2 amino acid substitutions, and/or a light chain variable region comprising a VLCDR3 comprising the amino acid sequence of SEQ ID NO: 86, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 59; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 86.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 59; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 86.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 59; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 86.

Amino acid substitutions may be made to provide variant antibodies with Fab regions derived from ADT1-4-117, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 59 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 86 optionally comprising 1 or 2 amino acid substitutions. The amino acid substitutions may be conservative amino acid substitutions.

The antibodies may alternatively consist of the specified sequences (with or without amino acid substitutions).

### ADT1-4-19 and Fragments and Variants Thereof

Certain embodiments relate to the antibody ADT1-4-19 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 60 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 87. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 60 and/or a light chain variable region comprising a VLCDR3 comprising an amino acid sequence having at least 90% identity to the amino acid sequence of SEQ ID NO: 87. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 60 and/or a light chain variable region comprising a VLCDR3 comprising the amino acid sequence of SEQ ID NO: 87.

Amino acid substitutions may be made to provide variant antibodies comprising Fab regions derived from ADT1-4-19, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 60, optionally comprising 1 or 2 amino acid substitutions, and/or a light chain variable region comprising a VLCDR3 comprising the amino acid sequence of SEQ ID NO: 87, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 60; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 87.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 60; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 87.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 60; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 87.

Amino acid substitutions may be made to provide variant antibodies with Fab regions derived from ADT1-4-19, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 60 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 87 optionally comprising 1 or 2 amino acid substitutions. The amino acid substitutions may be conservative amino acid substitutions.

The antibodies may alternatively consist of the specified sequences (with or without amino acid substitutions).

### ADT1-4-21 and Fragments and Variants Thereof

Certain embodiments relate to the antibody ADT1-4-21 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 61 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 88. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 61 and/or a light chain variable region comprising a VLCDR3 comprising an amino acid sequence having at least 90% identity to the amino acid sequence of SEQ ID NO: 88. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 61 and/or a light chain variable region comprising a VLCDR3 comprising the amino acid sequence of SEQ ID NO: 88.

Amino acid substitutions may be made to provide variant antibodies with Fab regions derived from ADT1-4-21, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 61, optionally comprising 1 or 2 amino acid substitutions, and/or a light chain variable region comprising a VLCDR3 comprising the amino acid sequence of SEQ ID NO: 88, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 52, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 61; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 88.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 52, a VHCDR2 comprising at least 90% identity to the amino acid sequence of SEQ ID NO: 53 and a VHCDR3 comprising at least 90% identity to the amino acid sequence of SEQ ID NO: 61; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 88.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 52, a VHCDR2 comprising the amino acid sequence of SEQ ID NO: 53 and a VHCDR3 comprising the amino acid sequence of SEQ ID NO: 61; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 88.

Amino acid substitutions may be made to provide variant antibodies comprising Fab regions derived from ADT1-4-21, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 52 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 61 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 88 optionally comprising 1 or 2 amino acid substitutions. The amino acid substitutions may be conservative amino acid substitutions.

The antibodies may alternatively consist of the specified sequences (with or without amino acid substitutions).

### ADT1-4-31 and Fragments and Variants Thereof

Certain embodiments relate to the antibody ADT1-4-31 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 62 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 89. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 62 and/or a light chain variable region comprising a VLCDR3 comprising an amino acid sequence having at least 90% identity to the amino acid sequence of SEQ ID NO: 89. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 62 and/or a light chain variable region comprising a VLCDR3 comprising the amino acid sequence of SEQ ID NO: 89.

Amino acid substitutions may be made to provide variant antibodies having Fab regions derived from ADT1-4-31, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 62, optionally comprising 1 or 2 amino acid substitutions, and/or a light chain variable region comprising a VLCDR3 comprising the amino acid sequence of SEQ ID NO: 89, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 62; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 89.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 62; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 89.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 62; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 89.

Amino acid substitutions may be made to provide variant antibodies having Fab regions derived from ADT1-4-31, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 62 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 89 optionally comprising 1 or 2 amino acid substitutions. The amino acid substitutions may be conservative amino acid substitutions.

The antibodies may alternatively consist of the specified sequences (with or without amino acid substitutions).

### ADT1-4-139 and Fragments and Variants Thereof

Certain embodiments relate to the antibody ADT1-4-139 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 63 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 90. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 63 and/or a light chain variable region comprising a VLCDR3 comprising an amino acid sequence having at least 90% identity to the amino acid sequence of SEQ ID NO: 90. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 63 and/or a light chain variable region comprising a VLCDR3 comprising the amino acid sequence of SEQ ID NO: 90.

Amino acid substitutions may be made to provide variant antibodies having Fab regions derived from ADT1-4-139, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 63, optionally comprising 1 or 2 amino acid substitutions, and/or a light chain variable region comprising a VLCDR3 comprising the amino acid sequence of SEQ ID NO: 90, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 63; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 90.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 63; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 90.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 63; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 90.

Amino acid substitutions may be made to provide variant antibodies having Fab regions derived from ADT1-4-139, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 63 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 90 optionally comprising 1 or 2 amino acid substitutions. The amino acid substitutions may be conservative amino acid substitutions.

The antibodies may alternatively consist of the specified sequences (with or without amino acid substitutions).

### ADT1-4-4 and Fragments and Variants Thereof

Certain embodiments relate to the antibody ADT1-4-4 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 64 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 91. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 64 and/or a light chain variable region comprising a VLCDR3 comprising an amino acid sequence having at least 90% identity to the amino acid sequence of SEQ ID NO: 91. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 64 and/or a light chain variable region comprising a VLCDR3 comprising the amino acid sequence of SEQ ID NO: 91.

Amino acid substitutions may be made to provide variant antibodies having Fab regions derived from ADT1-4-4, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 64, optionally comprising 1 or 2 amino acid substitutions, and/or a light chain variable region comprising a VLCDR3 comprising the amino acid sequence of SEQ ID NO: 91, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 64; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 91.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 64; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 91.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 64; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 91.

Amino acid substitutions may be made to provide variant antibodies having Fab regions derived from ADT1-4-4, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 64 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 91 optionally comprising 1 or 2 amino acid substitutions. The amino acid substitutions may be conservative amino acid substitutions.

The antibodies may alternatively consist of the specified sequences (with or without amino acid substitutions).

### ADT1-4-143 and Fragments and Variants Thereof

Certain embodiments relate to the antibody ADT1-4-143 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 65 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 92. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 65 and/or a light chain variable region comprising a VLCDR3 comprising an amino acid sequence having at least 90% identity to the amino acid sequence of SEQ ID NO: 92. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 65 and/or a light chain variable region comprising a VLCDR3 comprising the amino acid sequence of SEQ ID NO: 92.

Amino acid substitutions may be made to provide variant antibodies having Fab regions derived from ADT1-4-143, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 65, optionally comprising 1 or 2 amino acid substitutions, and/or a light chain variable region comprising a VLCDR3 comprising the amino acid sequence of SEQ ID NO: 92, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 65; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 92.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 65; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 92.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 65; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 92.

Amino acid substitutions may be made to provide variant antibodies having Fab regions derived from ADT1-4-143, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 65 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 92 optionally comprising 1 or 2 amino acid substitutions. The amino acid substitutions may be conservative amino acid substitutions.

The antibodies may alternatively consist of the specified sequences (with or without amino acid substitutions).

### ADT1-4-53 and Fragments and Variants Thereof

Certain embodiments relate to the antibody ADT1-4-53 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 66 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 93. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 66 and/or a light chain variable region comprising a VLCDR3 comprising an amino acid sequence having at least 90% identity to the amino acid sequence of SEQ ID NO: 93. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 66 and/or a light chain variable region comprising a VLCDR3 comprising the amino acid sequence of SEQ ID NO: 93.

Amino acid substitutions may be made to provide variant antibodies having Fab regions derived from ADT1-4-53, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 66, optionally comprising 1 or 2 amino acid substitutions, and/or a light chain variable region comprising a VLCDR3 comprising the amino acid sequence of SEQ ID NO: 93, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 52, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 66; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 93.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 52, a VHCDR2 comprising at least 90% identity to the amino acid sequence of SEQ ID NO: 53 and a VHCDR3 comprising at least 90% identity to the amino acid sequence of SEQ ID NO: 66; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 93.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 52, a VHCDR2 comprising the amino acid sequence of SEQ ID NO: 53 and a VHCDR3 comprising the amino acid sequence of SEQ ID NO: 66; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 93.

Amino acid substitutions may be made to provide variant antibodies having Fab regions derived from ADT1-4-53, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 52 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 66 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 93 optionally comprising 1 or 2 amino acid substitutions. The amino acid substitutions may be conservative amino acid substitutions.

The antibodies may alternatively consist of the specified sequences (with or without amino acid substitutions).

### ADT1-4-173 and Fragments and Variants Thereof

Certain embodiments relate to the antibody ADT1-4-173 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 67 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 94. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 67 and/or a light chain variable region comprising a VLCDR3 comprising an amino acid sequence having at least 90% identity to the amino acid sequence of SEQ ID NO: 94. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 67 and/or a light chain variable region comprising a VLCDR3 comprising the amino acid sequence of SEQ ID NO: 94.

Amino acid substitutions may be made to provide variant antibodies having Fab regions derived from ADT1-4-173, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 67, optionally comprising 1 or 2 amino acid substitutions, and/or a light chain variable region comprising a VLCDR3 comprising the amino acid sequence of SEQ ID NO: 94, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 67; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 94.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 67; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 94.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 67; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 94.

Amino acid substitutions may be made to provide variant antibodies having Fab regions derived from ADT1-4-173, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 67 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 94 optionally comprising 1 or 2 amino acid substitutions. The amino acid substitutions may be conservative amino acid substitutions.

The antibodies may alternatively consist of the specified sequences (with or without amino acid substitutions).

### ADT1-4-2 and Fragments and Variants Thereof

Certain embodiments relate to the antibody ADT1-4-2 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 68 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 95. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 68 and/or a light chain variable region comprising a VLCDR3 comprising an amino acid sequence having at least 90% identity to the amino acid sequence of SEQ ID NO: 95. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 68 and/or a light chain variable region comprising a VLCDR3 comprising the amino acid sequence of SEQ ID NO: 95.

Amino acid substitutions may be made to provide variant antibodies having Fab regions derived from ADT1-4-2, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 68, optionally comprising 1 or 2 amino acid substitutions, and/or a light chain variable region comprising a VLCDR3 comprising the amino acid sequence of SEQ ID NO: 95, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 68; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 95.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 68; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 95.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 68; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 95.

Amino acid substitutions may be made to provide variant antibodies having Fab regions derived from ADT1-4-2, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 68 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 95 optionally comprising 1 or 2 amino acid substitutions. The amino acid substitutions may be conservative amino acid substitutions.

The antibodies may alternatively consist of the specified sequences (with or without amino acid substitutions).

### ADT1-4-8 and Fragments and Variants Thereof

Certain embodiments relate to the antibody ADT1-4-8 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 69 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 96. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 69 and/or a light chain variable region comprising a VLCDR3 comprising an amino acid sequence having at least 90% identity to the amino acid sequence of SEQ ID NO: 96. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 69 and/or a light chain variable region comprising a VLCDR3 comprising the amino acid sequence of SEQ ID NO: 96.

Amino acid substitutions may be made to provide variant antibodies having Fab regions derived from ADT1-4-8, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 69, optionally comprising 1 or 2 amino acid substitutions, and/or a light chain variable region comprising a VLCDR3 comprising the amino acid sequence of SEQ ID NO: 96, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 69; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 96.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 69; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 96.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 69; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 96.

Amino acid substitutions may be made to provide variant antibodies having Fab regions derived from ADT1-4-8, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 69 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 96 optionally comprising 1 or 2 amino acid substitutions. The amino acid substitutions may be conservative amino acid substitutions.

The antibodies may alternatively consist of the specified sequences (with or without amino acid substitutions).

### ADT1-4-82 and Fragments and Variants Thereof

Certain embodiments relate to the antibody ADT1-4-82 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 70 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 97. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 70 and/or a light chain variable region comprising a VLCDR3 comprising an amino acid sequence having at least 90% identity to the amino acid sequence of SEQ ID NO: 97. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 70 and/or a light chain variable region comprising a VLCDR3 comprising the amino acid sequence of SEQ ID NO: 97.

Amino acid substitutions may be made to provide variant antibodies having Fab regions derived from ADT1-4-82, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 70, optionally comprising 1 or 2 amino acid substitutions, and/or a light chain variable region comprising a VLCDR3 comprising the amino acid sequence of SEQ ID NO: 97, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 70; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 97.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 70; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 97.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 70; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 97.

Amino acid substitutions may be made to provide variant antibodies having Fab regions derived from ADT1-4-82, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 70 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 97 optionally comprising 1 or 2 amino acid substitutions. The amino acid substitutions may be conservative amino acid substitutions.

The antibodies may alternatively consist of the specified sequences (with or without amino acid substitutions).

### ADT1-4-83 and Fragments and Variants Thereof

Certain embodiments relate to the antibody ADT1-4-83 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 71 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 98. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 71 and/or a light chain variable region comprising a VLCDR3 comprising an amino acid sequence having at least 90% identity to the amino acid sequence of SEQ ID NO: 98. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 71 and/or a light chain variable region comprising a VLCDR3 comprising the amino acid sequence of SEQ ID NO: 98.

Amino acid substitutions may be made to provide variant antibodies having Fab regions derived from ADT1-4-83, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 71, optionally comprising 1 or 2 amino acid substitutions, and/or a light chain variable region comprising a VLCDR3 comprising the amino acid sequence of SEQ ID NO: 98, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 71; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 98.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 71; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 98.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 71; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 98.

Amino acid substitutions may be made to provide variant antibodies having Fab regions derived from ADT1-4-83, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 71 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 98 optionally comprising 1 or 2 amino acid substitutions. The amino acid substitutions may be conservative amino acid substitutions.

The antibodies may alternatively consist of the specified sequences (with or without amino acid substitutions).

### ADT1-4-3 and Fragments and Variants Thereof

Certain embodiments relate to the antibody ADT1-4-3 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 72 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 99. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 72 and/or a light chain variable region comprising a VLCDR3 comprising an amino acid sequence having at least 90% identity to the amino acid sequence of SEQ ID NO: 99. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 72 and/or a light chain variable region comprising a VLCDR3 comprising the amino acid sequence of SEQ ID NO: 99.

Amino acid substitutions may be made to provide variant antibodies having Fab regions derived from ADT1-4-3, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 72, optionally comprising 1 or 2 amino acid substitutions, and/or a light chain variable region comprising a VLCDR3 comprising the amino acid sequence of SEQ ID NO: 99, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 72; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 99.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 72; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 99.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 72; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 99.

Amino acid substitutions may be made to provide variant antibodies having Fab regions derived from ADT1-4-3, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 72 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 99 optionally comprising 1 or 2 amino acid substitutions. The amino acid substitutions may be conservative amino acid substitutions.

The antibodies may alternatively consist of the specified sequences (with or without amino acid substitutions).

### ADT1-4-84 and Fragments and Variants Thereof

Certain embodiments relate to the antibody ADT1-4-84 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 73 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 100. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 73 and/or a light chain variable region comprising a VLCDR3 comprising an amino acid sequence having at least 90% identity to the amino acid sequence of SEQ ID NO: 100. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 73 and/or a light chain variable region comprising a VLCDR3 comprising the amino acid sequence of SEQ ID NO: 100.

Amino acid substitutions may be made to provide variant antibodies having Fab regions derived from ADT1-4-84, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 73, optionally comprising 1 or 2 amino acid substitutions, and/or a light chain variable region comprising a VLCDR3 comprising the amino acid sequence of SEQ ID NO: 100, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 73; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 100.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 73; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 100.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 73; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 100.

Amino acid substitutions may be made to provide variant antibodies having Fab regions derived from ADT1-4-84, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 73 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 100 optionally comprising 1 or 2 amino acid substitutions. The amino acid substitutions may be conservative amino acid substitutions.

The antibodies may alternatively consist of the specified sequences (with or without amino acid substitutions).

### ADT1-4-86 and Fragments and Variants Thereof

Certain embodiments relate to the antibody ADT1-4-86 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 74 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 101. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 74 and/or a light chain variable region comprising a VLCDR3 comprising an amino acid sequence having at least 90% identity to the amino acid sequence of SEQ ID NO: 101. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 74 and/or a light chain variable region comprising a VLCDR3 comprising the amino acid sequence of SEQ ID NO: 101.

Amino acid substitutions may be made to provide variant antibodies having Fab regions derived from ADT1-4-86, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 74, optionally comprising 1 or 2 amino acid substitutions, and/or a light chain variable region comprising a VLCDR3 comprising the amino acid sequence of SEQ ID NO: 101, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 52, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 74; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 101.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 52, a VHCDR2 comprising at least 90% identity to the amino acid sequence of SEQ ID NO: 53 and a VHCDR3 comprising at least 90% identity to the amino acid sequence of SEQ ID NO: 74; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 101.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 52, a VHCDR2 comprising the amino acid sequence of SEQ ID NO: 53 and a VHCDR3 comprising the amino acid sequence of SEQ ID NO: 74; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 101.

Amino acid substitutions may be made to provide variant antibodies having Fab regions derived from ADT1-4-86, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 52 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 74 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 101 optionally comprising 1 or 2 amino acid substitutions. The amino acid substitutions may be conservative amino acid substitutions.

The antibodies may alternatively consist of the specified sequences (with or without amino acid substitutions).

### ADT1-4-95 and Fragments and Variants Thereof

Certain embodiments relate to the antibody ADT1-4-95 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 75 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 102. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 75 and/or a light chain variable region comprising a VLCDR3 comprising an amino acid sequence having at least 90% identity to the amino acid sequence of SEQ ID NO: 102. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 75 and/or a light chain variable region comprising a VLCDR3 comprising the amino acid sequence of SEQ ID NO: 102.

Amino acid substitutions may be made to provide variant antibodies having Fab regions derived from ADT1-4-95, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 75, optionally comprising 1 or 2 amino acid substitutions, and/or a light chain variable region comprising a VLCDR3 comprising the amino acid sequence of SEQ ID NO: 102, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 75; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 102.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 75; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 102.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 75; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 102.

Amino acid substitutions may be made to provide variant antibodies having Fab regions derived from ADT1-4-95, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 75 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 102 optionally comprising 1 or 2 amino acid substitutions. The amino acid substitutions may be conservative amino acid substitutions.

The antibodies may alternatively consist of the specified sequences (with or without amino acid substitutions).

### ADT1-4-1 and Fragments and Variants Thereof

Certain embodiments relate to the antibody ADT1-4-1 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 76 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 103. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 76 and/or a light chain variable region comprising a VLCDR3 comprising an amino acid sequence having at least 90% identity to the amino acid sequence of SEQ ID NO: 103. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 76 and/or a light chain variable region comprising a VLCDR3 comprising the amino acid sequence of SEQ ID NO: 103.

Amino acid substitutions may be made to provide variant antibodies having Fab regions derived from ADT1-4-1, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 76, optionally comprising 1 or 2 amino acid substitutions, and/or a light chain variable region comprising a VLCDR3 comprising the amino acid sequence of SEQ ID NO: 103, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 76; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 103.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 76; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 103.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 76; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 103.

Amino acid substitutions may be made to provide variant antibodies having Fab regions derived from ADT1-4-1, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 76 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 103 optionally comprising 1 or 2 amino acid substitutions. The amino acid substitutions may be conservative amino acid substitutions.

The antibodies may alternatively consist of the specified sequences (with or without amino acid substitutions).

### ADT1-4-6 and Fragments and Variants Thereof

Certain embodiments relate to the antibody ADT1-4-6 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 77 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 104. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 77 and/or a light chain variable region comprising a VLCDR3 comprising an amino acid sequence having at least 90% identity to the amino acid sequence of SEQ ID NO: 104. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 77 and/or a light chain variable region comprising a VLCDR3 comprising the amino acid sequence of SEQ ID NO: 104.

Amino acid substitutions may be made to provide variant antibodies having Fab regions derived from ADT1-4-6, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 77, optionally comprising 1 or 2 amino acid substitutions, and/or a light chain variable region comprising a VLCDR3 comprising the amino acid sequence of SEQ ID NO: 104, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 77; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 104.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 77; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 104.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 77; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 104.

Amino acid substitutions may be made to provide variant antibodies having Fab regions derived from ADT1-4-6, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 77 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 104 optionally comprising 1 or 2 amino acid substitutions. The amino acid substitutions may be conservative amino acid substitutions.

The antibodies may alternatively consist of the specified sequences (with or without amino acid substitutions).

### ADT1-4-138 and Fragments and Variants Thereof

Certain embodiments relate to the antibody ADT1-4-138 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 78 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 105. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 78 and/or a light chain variable region comprising a VLCDR3 comprising an amino acid sequence having at least 90% identity to the amino acid sequence of SEQ ID NO: 105. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 78 and/or a light chain variable region comprising a VLCDR3 comprising the amino acid sequence of SEQ ID NO: 105.

Amino acid substitutions may be made to provide variant antibodies having Fab regions derived from ADT1-4-138, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 78, optionally comprising 1 or 2 amino acid substitutions, and/or a light chain variable region comprising a VLCDR3 comprising the amino acid sequence of SEQ ID NO: 105, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 51, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 53 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 78; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 79, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 80 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 105.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 51, a VHCDR2 comprising at least 90% identity to the amino acid sequence of SEQ ID NO: 53 and a VHCDR3 comprising at least 90% identity to the amino acid sequence of SEQ ID NO: 78; and/or
a light chain variable region comprising a VLCDR1 comprising the at least 90% identity to the amino acid sequence of SEQ ID NO: 79, a VLCDR2 comprising at least 90% identity to the amino acid sequence of SEQ ID NO: 80 and a VLCDR3 comprising at least 90% identity to the amino acid sequence of SEQ ID NO: 105.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51, a VHCDR2 comprising the amino acid sequence of SEQ ID NO: 53 and a VHCDR3 comprising the amino acid sequence of SEQ ID NO: 78; and
a light chain variable region comprising a VLCDR1 comprising the amino acid sequence of SEQ ID NO: 79, a VLCDR2 comprising the amino acid sequence of SEQ ID NO: 80 and a VLCDR3 comprising the amino acid sequence of SEQ ID NO: 105.

Amino acid substitutions may be made to provide variant antibodies having Fab regions derived from ADT1-4-138, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 51 optionally comprising 1 or 2 amino acid substitutions, a VHCDR2 comprising the amino acid sequence of SEQ ID NO: 53 optionally comprising 1 or 2 amino acid substitutions, and a VHCDR3 comprising the amino acid sequence of SEQ ID NO: 78 optionally comprising 1 or 2 amino acid substitutions; and/or
a light chain variable region comprising a VLCDR1 comprising the amino acid sequence of SEQ ID NO: 79 optionally comprising 1 or 2 amino acid substitutions, a VLCDR2 comprising the amino acid sequence of SEQ ID NO: 80 optionally comprising 1 or 2 amino acid substitutions and a VLCDR3 comprising the amino acid sequence of SEQ ID NO: 105 optionally comprising 1 or 2 amino acid substitutions. The amino acid substitutions may be conservative amino acid substitutions.

The antibodies may alternatively consist of the specified sequences (with or without amino acid substitutions).

Antibodies Comprising Heavy and/or Light Chain Variable Regions Derived from ADT1-4

Multispecific antibodies are provided herein, the anti-TCR delta variable 1 (anti-Vδ1) binding Fab region comprising:
a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 2 to 25; and/or
a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 27 to 50.

Also provided are multispecific antibodies, the anti-TCR delta variable 1 (anti-Vδ1) binding Fab region comprising:
a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 2 to 24; and/or
a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 27 to 49.

Also provided are multispecific antibodies, the anti-TCR delta variable 1 (anti-Vδ1) binding Fab region comprising:
a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 5, 7, 8, 9, 12, 13, 15, 21, 23 and 24; and/or
a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 30, 32, 33, 34, 37, 38, 40, 46, 48 and 49.

Also provided are multispecific antibodies, the anti-TCR delta variable 1 (anti-Vδ1) binding Fab region comprising:
a heavy chain variable region comprising or consisting of a sequence having at least 90% identity to a sequence selected from the group consisting of SEQ ID NOs: 2 to 25; and/or
a light chain variable region comprising or consisting of a sequence having at least 90% identity to a sequence selected from the group consisting of SEQ ID NOs: 27 to 50.

Also provided are multispecific antibodies, the anti-TCR delta variable 1 (anti-Vδ1) binding Fab region comprising:
a heavy chain variable region comprising or consisting of a sequence having at least 90% identity to a sequence selected from the group consisting of SEQ ID NOs: 2 to 24; and/or
a light chain variable region comprising or consisting of a sequence having at least 90% identity to a sequence selected from the group consisting of SEQ ID NOs: 27 to 49.

Also provided are multispecific antibodies, the anti-TCR delta variable 1 (anti-Vδ1) binding Fab region comprising:
a heavy chain variable region comprising or consisting of a sequence having at least 90% identity to a sequence selected from the group consisting of SEQ ID NOs: 5, 7, 8, 9, 12, 13, 15, 21, 23 and 24; and/or
a light chain variable region comprising or consisting of a sequence having at least 90% identity to a sequence selected from the group consisting of SEQ ID NOs: 30, 32, 33, 34, 37, 38, 40, 46, 48 and 49.

Optionally the antibodies above retain the corresponding CDR sequences such that any variability in the VH and VL sequences occurs in the framework regions.

Also provided are multispecific antibodies, the anti-TCR delta variable 1 (anti-Vδ1) binding Fab region comprising:
a heavy chain variable region comprising or consisting of a sequence selected from the group consisting of SEQ ID NOs: 2 to 25; and/or
a light chain variable region comprising or consisting of a sequence selected from the group consisting of SEQ ID NOs: 27 to 50.

Also provided are multispecific antibodies, the anti-TCR delta variable 1 (anti-Vδ1) binding Fab region comprising:
a heavy chain variable region comprising or consisting of a sequence selected from the group consisting of SEQ ID NOs: 2 to 24; and/or
a light chain variable region comprising or consisting of a sequence selected from the group consisting of SEQ ID NOs: 27 to 49.

Also provided are multispecific antibodies, the anti-TCR delta variable 1 (anti-Vδ1) binding Fab region comprising:
a heavy chain variable region comprising or consisting of a sequence selected from the group consisting of SEQ ID NOs: 5, 7, 8, 9, 12, 13, 15, 21, 23 and 24; and/or
a light chain variable region comprising or consisting of a sequence selected from the group consisting of SEQ ID NOs: 30, 32, 33, 34, 37, 38, 40, 46, 48 and 49.

In some embodiments, multispecific antibodies are provided, the anti-TCR delta variable 1 (anti-Vδ1) binding Fab region comprising:
a) a VH comprising or consisting of an amino acid sequence that is at least 90% identical to the amino acid sequence of SEQ ID NO: 2 and a VL comprising or consisting of an amino acid sequence that is at least 90% identical to the amino acid sequence of SEQ ID NO: 27;
b) a VH comprising or consisting of an amino acid sequence that is at least 90% identical to the amino acid sequence of SEQ ID NO: 3 and a VL comprising or consisting of an amino acid sequence that is at least 90% identical to the amino acid sequence of SEQ ID NO: 28;
c) a VH comprising or consisting of an amino acid sequence that is at least 90% identical to the amino acid sequence of SEQ ID NO: 4 and a VL comprising or consisting of an amino acid sequence that is at least 90% identical to the amino acid sequence of SEQ ID NO: 29;
d) a VH comprising or consisting of an amino acid sequence that is at least 90% identical to the amino acid sequence of SEQ ID NO: 5 and a VL comprising or consisting of an amino acid sequence that is at least 90% identical to the amino acid sequence of SEQ ID NO: 30;
e) a VH comprising or consisting of an amino acid sequence that is at least 90% identical to the amino acid sequence of SEQ ID NO: 6 and a VL comprising or consisting of an amino acid sequence that is at least 90% identical to the amino acid sequence of SEQ ID NO: 31;
f) a VH comprising or consisting of an amino acid sequence that is at least 90% identical to the amino acid sequence of SEQ ID NO: 7 and a VL comprising or consisting of an amino acid sequence that is at least 90% identical to the amino acid sequence of SEQ ID NO: 32;
g) a VH comprising or consisting of an amino acid sequence that is at least 90% identical to the amino acid sequence of SEQ ID NO: 8 and a VL comprising or consisting of an amino acid sequence that is at least 90% identical to the amino acid sequence of SEQ ID NO: 33;
h) a VH comprising or consisting of an amino acid sequence that is at least 90% identical to the amino acid sequence of SEQ ID NO: 9 and a VL comprising or consisting of an amino acid sequence that is at least 90% identical to the amino acid sequence of SEQ ID NO: 34;
i) a VH comprising or consisting of an amino acid sequence that is at least 90% identical to the amino acid sequence of SEQ ID NO: 10 and a VL comprising or consisting of an amino acid sequence that is at least 90% identical to the amino acid sequence of SEQ ID NO: 35;
j) a VH comprising or consisting of an amino acid sequence that is at least 90% identical to the amino acid sequence of SEQ ID NO: 11 and a VL comprising or consisting of an amino acid sequence that is at least 90% identical to the amino acid sequence of SEQ ID NO: 36;
k) a VH comprising or consisting of an amino acid sequence that is at least 90% identical to the amino acid sequence of SEQ ID NO: 12 and a VL comprising or consisting of an amino acid sequence that is at least 90% identical to the amino acid sequence of SEQ ID NO: 37;
l) a VH comprising or consisting of an amino acid sequence that is at least 90% identical to the amino acid sequence of SEQ ID NO: 13 and a VL comprising or consisting of an amino acid sequence that is at least 90% identical to the amino acid sequence of SEQ ID NO: 38;
m) a VH comprising or consisting of an amino acid sequence that is at least 90% identical to the amino acid sequence of SEQ ID NO: 14 and a VL comprising or consisting of an amino acid sequence that is at least 90% identical to the amino acid sequence of SEQ ID NO: 39;
n) a VH comprising or consisting of an amino acid sequence that is at least 90% identical to the amino acid sequence of SEQ ID NO: 15 and a VL comprising or consisting of an amino acid sequence that is at least 90% identical to the amino acid sequence of SEQ ID NO: 40;
o) a VH comprising or consisting of an amino acid sequence that is at least 90% identical to the amino acid sequence of SEQ ID NO: 16 and a VL comprising or consisting of an amino acid sequence that is at least 90% identical to the amino acid sequence of SEQ ID NO: 41;
p) a VH comprising or consisting of an amino acid sequence that is at least 90% identical to the amino acid sequence of SEQ ID NO: 17 and a VL comprising or consisting of an amino acid sequence that is at least 90% identical to the amino acid sequence of SEQ ID NO: 42;
q) a VH comprising or consisting of an amino acid sequence that is at least 90% identical to the amino acid sequence of SEQ ID NO: 18 and a VL comprising or consisting of an amino acid sequence that is at least 90% identical to the amino acid sequence of SEQ ID NO: 43;
r) a VH comprising or consisting of an amino acid sequence that is at least 90% identical to the amino acid sequence of SEQ ID NO: 19 and a VL comprising or consisting of an amino acid sequence that is at least 90% identical to the amino acid sequence of SEQ ID NO: 44;
s) a VH comprising or consisting of an amino acid sequence that is at least 90% identical to the amino acid sequence of SEQ ID NO: 20 and a VL comprising or consisting of an amino acid sequence that is at least 90% identical to the amino acid sequence of SEQ ID NO: 45;
t) a VH comprising or consisting of an amino acid sequence that is at least 90% identical to the amino acid sequence of SEQ ID NO: 21 and a VL comprising or consisting of an amino acid sequence that is at least 90% identical to the amino acid sequence of SEQ ID NO: 46;
u) a VH comprising or consisting of an amino acid sequence that is at least 90% identical to the amino acid sequence of SEQ ID NO: 22 and a VL comprising or consisting of an amino acid sequence that is at least 90% identical to the amino acid sequence of SEQ ID NO: 47;
v) a VH comprising or consisting of an amino acid sequence that is at least 90% identical to the amino acid sequence of SEQ ID NO: 23 and a VL comprising or consisting of an amino acid sequence that is at least 90% identical to the amino acid sequence of SEQ ID NO: 48;
w) a VH comprising or consisting of an amino acid sequence that is at least 90% identical to the amino acid sequence of SEQ ID NO: 24 and a VL comprising or consisting of an amino acid sequence that is at least 90% identical to the amino acid sequence of SEQ ID NO: 49; or
x) a VH comprising or consisting of an amino acid sequence that is at least 90% identical to the amino acid sequence of SEQ ID NO: 25 and a VL comprising or consisting of an amino acid sequence that is at least 90% identical to the amino acid sequence of SEQ ID NO: 50.

Optionally the antibodies above retain the corresponding CDR sequences such that any variability in the VH and VL sequences occurs in the framework regions.

In some embodiments, multispecific antibodies are provided, the anti-TCR delta variable 1 (anti-Vδ1) binding Fab region comprising:
a) a VH comprising or consisting of an amino acid sequence that is at least 96% identical to the amino acid sequence of SEQ ID NO: 2 and a VL comprising or consisting of an amino acid sequence that is at least 96% identical to the amino acid sequence of SEQ ID NO: 27;
b) a VH comprising or consisting of an amino acid sequence that is at least 96% identical to the amino acid sequence of SEQ ID NO: 3 and a VL comprising or consisting of an amino acid sequence that is at least 96% identical to the amino acid sequence of SEQ ID NO: 28;
c) a VH comprising or consisting of an amino acid sequence that is at least 96% identical to the amino acid sequence of SEQ ID NO: 4 and a VL comprising or consisting of an amino acid sequence that is at least 96% identical to the amino acid sequence of SEQ ID NO: 29;
d) a VH comprising or consisting of an amino acid sequence that is at least 96% identical to the amino acid sequence of SEQ ID NO: 5 and a VL comprising or consisting of an amino acid sequence that is at least 96% identical to the amino acid sequence of SEQ ID NO: 30;
e) a VH comprising or consisting of an amino acid sequence that is at least 96% identical to the amino acid sequence of SEQ ID NO: 6 and a VL comprising or consisting of an amino acid sequence that is at least 96% identical to the amino acid sequence of SEQ ID NO: 31;
f) a VH comprising or consisting of an amino acid sequence that is at least 96% identical to the amino acid sequence of SEQ ID NO: 7 and a VL comprising or consisting of an amino acid sequence that is at least 96% identical to the amino acid sequence of SEQ ID NO: 32;
g) a VH comprising or consisting of an amino acid sequence that is at least 96% identical to the amino acid sequence of SEQ ID NO: 8 and a VL comprising or consisting of an amino acid sequence that is at least 96% identical to the amino acid sequence of SEQ ID NO: 33;
h) a VH comprising or consisting of an amino acid sequence that is at least 96% identical to the amino acid sequence of SEQ ID NO: 9 and a VL comprising or consisting of an amino acid sequence that is at least 96% identical to the amino acid sequence of SEQ ID NO: 34;
i) a VH comprising or consisting of an amino acid sequence that is at least 96% identical to the amino acid sequence of SEQ ID NO: 10 and a VL comprising or consisting of an amino acid sequence that is at least 96% identical to the amino acid sequence of SEQ ID NO: 35;
j) a VH comprising or consisting of an amino acid sequence that is at least 96% identical to the amino acid sequence of SEQ ID NO: 11 and a VL comprising or consisting of an amino acid sequence that is at least 96% identical to the amino acid sequence of SEQ ID NO: 36;
k) a VH comprising or consisting of an amino acid sequence that is at least 96% identical to the amino acid sequence of SEQ ID NO: 12 and a VL comprising or consisting of an amino acid sequence that is at least 96% identical to the amino acid sequence of SEQ ID NO: 37;
l) a VH comprising or consisting of an amino acid sequence that is at least 96% identical to the amino acid sequence of SEQ ID NO: 13 and a VL comprising or consisting of an amino acid sequence that is at least 96% identical to the amino acid sequence of SEQ ID NO: 38;
m) a VH comprising or consisting of an amino acid sequence that is at least 96% identical to the amino acid sequence of SEQ ID NO: 14 and a VL comprising or consisting of an amino acid sequence that is at least 96% identical to the amino acid sequence of SEQ ID NO: 39;
n) a VH comprising or consisting of an amino acid sequence that is at least 96% identical to the amino acid sequence of SEQ ID NO: 15 and a VL comprising or consisting of an amino acid sequence that is at least 96% identical to the amino acid sequence of SEQ ID NO: 40;
o) a VH comprising or consisting of an amino acid sequence that is at least 96% identical to the amino acid sequence of SEQ ID NO: 16 and a VL comprising or consisting of an amino acid sequence that is at least 96% identical to the amino acid sequence of SEQ ID NO: 41;
p) a VH comprising or consisting of an amino acid sequence that is at least 96% identical to the amino acid sequence of SEQ ID NO: 17 and a VL comprising or consisting of an amino acid sequence that is at least 96% identical to the amino acid sequence of SEQ ID NO: 42;
q) a VH comprising or consisting of an amino acid sequence that is at least 96% identical to the amino acid sequence of SEQ ID NO: 18 and a VL comprising or consisting of an amino acid sequence that is at least 96% identical to the amino acid sequence of SEQ ID NO: 43;
r) a VH comprising or consisting of an amino acid sequence that is at least 96% identical to the amino acid sequence of SEQ ID NO: 19 and a VL comprising or consisting of an amino acid sequence that is at least 96% identical to the amino acid sequence of SEQ ID NO: 44;
s) a VH comprising or consisting of an amino acid sequence that is at least 96% identical to the amino acid sequence of SEQ ID NO: 20 and a VL comprising or consisting of an amino acid sequence that is at least 96% identical to the amino acid sequence of SEQ ID NO: 45;
t) a VH comprising or consisting of an amino acid sequence that is at least 96% identical to the amino acid sequence of SEQ ID NO: 21 and a VL comprising or consisting of an amino acid sequence that is at least 96% identical to the amino acid sequence of SEQ ID NO: 46;
u) a VH comprising or consisting of an amino acid sequence that is at least 96% identical to the amino acid sequence of SEQ ID NO: 22 and a VL comprising or consisting of an amino acid sequence that is at least 96% identical to the amino acid sequence of SEQ ID NO: 47;
v) a VH comprising or consisting of an amino acid sequence that is at least 96% identical to the amino acid sequence of SEQ ID NO: 23 and a VL comprising or consisting of an amino acid sequence that is at least 96% identical to the amino acid sequence of SEQ ID NO: 48;
w) a VH comprising or consisting of an amino acid sequence that is at least 96% identical to the amino acid sequence of SEQ ID NO: 24 and a VL comprising or consisting of an amino acid sequence that is at least 96% identical to the amino acid sequence of SEQ ID NO: 49; or
x) a VH comprising or consisting of an amino acid sequence that is at least 96% identical to the amino acid sequence of SEQ ID NO: 25 and a VL comprising or consisting of an amino acid sequence that is at least 96% identical to the amino acid sequence of SEQ ID NO: 50.

Optionally the antibodies above retain the corresponding CDR sequences such that any variability in the VH and VL sequences occurs in the framework regions.

Optionally the antibodies above retain the corresponding CDR sequences such that any variability in the VH and VL sequences occurs in the framework regions.

In some embodiments, multispecific antibodies are provided, the anti-TCR delta variable 1 (anti-Vδ1) binding Fab region comprising:
a) a VH comprising or consisting of the amino acid sequence of SEQ ID NO: 2 and a VL comprising or consisting of the amino acid sequence of SEQ ID NO: 27 optionally comprising up to 5, up to 4, up to 3, up to 2 or 1 amino acid substitutions across both variable regions;
b) a VH comprising or consisting of the amino acid sequence of SEQ ID NO: 3 and a VL comprising or consisting of the amino acid sequence of SEQ ID NO: 28 optionally comprising up to 5, up to 4, up to 3, up to 2 or 1 amino acid substitutions across both variable regions;
c) a VH comprising or consisting of the amino acid sequence of SEQ ID NO: 4 and a VL comprising or consisting of the amino acid sequence of SEQ ID NO: 29 optionally comprising up to 5, up to 4, up to 3, up to 2 or 1 amino acid substitutions across both variable regions;
d) a VH comprising or consisting of the amino acid sequence of SEQ ID NO: 5 and a VL comprising or consisting of the amino acid sequence of SEQ ID NO: 30 optionally comprising up to 5, up to 4, up to 3, up to 2 or 1 amino acid substitutions across both variable regions;
e) a VH comprising or consisting of the amino acid sequence of SEQ ID NO: 6 and a VL comprising or consisting of the amino acid sequence of SEQ ID NO: 31 optionally comprising up to 5, up to 4, up to 3, up to 2 or 1 amino acid substitutions across both variable regions;
f) a VH comprising or consisting of the amino acid sequence of SEQ ID NO: 7 and a VL comprising or consisting of the amino acid sequence of SEQ ID NO: 32 optionally comprising up to 5, up to 4, up to 3, up to 2 or 1 amino acid substitutions across both variable regions;
g) a VH comprising or consisting of the amino acid sequence of SEQ ID NO: 8 and a VL comprising or consisting of the amino acid sequence of SEQ ID NO: 33 optionally comprising up to 5, up to 4, up to 3, up to 2 or 1 amino acid substitutions across both variable regions;
h) a VH comprising or consisting of the amino acid sequence of SEQ ID NO: 9 and a VL comprising or consisting of the amino acid sequence of SEQ ID NO: 34 optionally comprising up to 5, up to 4, up to 3, up to 2 or 1 amino acid substitutions across both variable regions;
i) a VH comprising or consisting of the amino acid sequence of SEQ ID NO: 10 and a VL comprising or consisting of the amino acid sequence of SEQ ID NO: 35 optionally comprising up to 5, up to 4, up to 3, up to 2 or 1 amino acid substitutions across both variable regions;
j) a VH comprising or consisting of the amino acid sequence of SEQ ID NO: 11 and a VL comprising or consisting of the amino acid sequence of SEQ ID NO: 36 optionally comprising up to 5, up to 4, up to 3, up to 2 or 1 amino acid substitutions across both variable regions;
k) a VH comprising or consisting of the amino acid sequence of SEQ ID NO: 12 and a VL comprising or consisting of the amino acid sequence of SEQ ID NO: 37 optionally comprising up to 5, up to 4, up to 3, up to 2 or 1 amino acid substitutions across both variable regions;
l) a VH comprising or consisting of the amino acid sequence of SEQ ID NO: 13 and a VL comprising or consisting of the amino acid sequence of SEQ ID NO: 38 optionally comprising up to 5, up to 4, up to 3, up to 2 or 1 amino acid substitutions across both variable regions;
m) a VH comprising or consisting of the amino acid sequence of SEQ ID NO: 14 and a VL comprising or consisting of the amino acid sequence of SEQ ID NO: 39 optionally comprising up to 5, up to 4, up to 3, up to 2 or 1 amino acid substitutions across both variable regions;
n) a VH comprising or consisting of the amino acid sequence of SEQ ID NO: 15 and a VL comprising or consisting of the amino acid sequence of SEQ ID NO: 40 optionally comprising up to 5, up to 4, up to 3, up to 2 or 1 amino acid substitutions across both variable regions;
o) a VH comprising or consisting of the amino acid sequence of SEQ ID NO: 16 and a VL comprising or consisting of the amino acid sequence of SEQ ID NO: 41 optionally comprising up to 5, up to 4, up to 3, up to 2 or 1 amino acid substitutions across both variable regions;
p) a VH comprising or consisting of the amino acid sequence of SEQ ID NO: 17 and a VL comprising or consisting of the amino acid sequence of SEQ ID NO: 42 optionally comprising up to 5, up to 4, up to 3, up to 2 or 1 amino acid substitutions across both variable regions;
q) a VH comprising or consisting of the amino acid sequence of SEQ ID NO: 18 and a VL comprising or consisting of the amino acid sequence of SEQ ID NO: 43 optionally comprising up to 5, up to 4, up to 3, up to 2 or 1 amino acid substitutions across both variable regions;
r) a VH comprising or consisting of the amino acid sequence of SEQ ID NO: 19 and a VL comprising or consisting of the amino acid sequence of SEQ ID NO: 44 optionally comprising up to 5, up to 4, up to 3, up to 2 or 1 amino acid substitutions across both variable regions;
s) a VH comprising or consisting of the amino acid sequence of SEQ ID NO: 20 and a VL comprising or consisting of the amino acid sequence of SEQ ID NO: 45 optionally comprising up to 5, up to 4, up to 3, up to 2 or 1 amino acid substitutions across both variable regions;
t) a VH comprising or consisting of the amino acid sequence of SEQ ID NO: 21 and a VL comprising or consisting of the amino acid sequence of SEQ ID NO: 46 optionally comprising up to 5, up to 4, up to 3, up to 2 or 1 amino acid substitutions across both variable regions;
u) a VH comprising or consisting of the amino acid sequence of SEQ ID NO: 22 and a VL comprising or consisting of the amino acid sequence of SEQ ID NO: 47 optionally comprising up to 5, up to 4, up to 3, up to 2 or 1 amino acid substitutions across both variable regions;
v) a VH comprising or consisting of the amino acid sequence of SEQ ID NO: 23 and a VL comprising or consisting of the amino acid sequence of SEQ ID NO: 48 optionally comprising up to 5, up to 4, up to 3, up to 2 or 1 amino acid substitutions across both variable regions;
w) a VH comprising or consisting of the amino acid sequence of SEQ ID NO: 24 and a VL comprising or consisting of the amino acid sequence of SEQ ID NO: 49 optionally comprising up to 5, up to 4, up to 3, up to 2 or 1 amino acid substitutions across both variable regions; or
x) a VH comprising or consisting of the amino acid sequence of SEQ ID NO: 25 and a VL comprising or consisting of the amino acid sequence of SEQ ID NO: 50 optionally comprising up to 5, up to 4, up to 3, up to 2 or 1 amino acid substitutions across both variable regions.

“Across both variable regions” means collectively, the antibody may comprise up to the specified number of substitutions in total when considering both the heavy and light chain variable regions. The amino acid substitutions may be conservative amino acid substitutions. In some embodiments, the substitutions (if present) may occur anywhere in the variable region sequences. In preferred embodiments, the substitutions (if present) may be limited to the framework regions. Accordingly, in some embodiments, the amino acid substitutions do not occur in a CDR sequence.

Optionally the antibodies above retain the corresponding CDR sequences such that any variability in the VH and VL sequences occurs in the framework regions.

In some embodiments, multispecific antibodies are provided, the anti-TCR delta variable 1 (anti-Vδ1) binding Fab region comprising:
a) a VH comprising or consisting of the amino acid sequence of SEQ ID NO: 2 and a VL comprising or consisting of the amino acid sequence of SEQ ID NO: 27;
b) a VH comprising or consisting of the amino acid sequence of SEQ ID NO: 3 and a VL comprising or consisting of the amino acid sequence of SEQ ID NO: 28;
c) a VH comprising or consisting of the amino acid sequence of SEQ ID NO: 4 and a VL comprising or consisting of the amino acid sequence of SEQ ID NO: 29;
d) a VH comprising or consisting of the amino acid sequence of SEQ ID NO: 5 and a VL comprising or consisting of the amino acid sequence of SEQ ID NO: 30;
e) a VH comprising or consisting of the amino acid sequence of SEQ ID NO: 6 and a VL comprising or consisting of the amino acid sequence of SEQ ID NO: 31;
f) a VH comprising or consisting of the amino acid sequence of SEQ ID NO: 7 and a VL comprising or consisting of the amino acid sequence of SEQ ID NO: 32;
g) a VH comprising or consisting of the amino acid sequence of SEQ ID NO: 8 and a VL comprising or consisting of the amino acid sequence of SEQ ID NO: 33;
h) a VH comprising or consisting of the amino acid sequence of SEQ ID NO: 9 and a VL comprising or consisting of the amino acid sequence of SEQ ID NO: 34;
i) a VH comprising or consisting of the amino acid sequence of SEQ ID NO: 10 and a VL comprising or consisting of the amino acid sequence of SEQ ID NO: 35;
j) a VH comprising or consisting of the amino acid sequence of SEQ ID NO: 11 and a VL comprising or consisting of the amino acid sequence of SEQ ID NO: 36;
k) a VH comprising or consisting of the amino acid sequence of SEQ ID NO: 12 and a VL comprising or consisting of the amino acid sequence of SEQ ID NO: 37;
l) a VH comprising or consisting of the amino acid sequence of SEQ ID NO: 13 and a VL comprising or consisting of the amino acid sequence of SEQ ID NO: 38;
m) a VH comprising or consisting of the amino acid sequence of SEQ ID NO: 14 and a VL comprising or consisting of the amino acid sequence of SEQ ID NO: 39;
n) a VH comprising or consisting of the amino acid sequence of SEQ ID NO: 15 and a VL comprising or consisting of the amino acid sequence of SEQ ID NO: 40;
o) a VH comprising or consisting of the amino acid sequence of SEQ ID NO: 16 and a VL comprising or consisting of the amino acid sequence of SEQ ID NO: 41;
p) a VH comprising or consisting of the amino acid sequence of SEQ ID NO: 17 and a VL comprising or consisting of the amino acid sequence of SEQ ID NO: 42;
q) a VH comprising or consisting of the amino acid sequence of SEQ ID NO: 18 and a VL comprising or consisting of the amino acid sequence of SEQ ID NO: 43;
r) a VH comprising or consisting of the amino acid sequence of SEQ ID NO: 19 and a VL comprising or consisting of the amino acid sequence of SEQ ID NO: 44;
s) a VH comprising or consisting of the amino acid sequence of SEQ ID NO: 20 and a VL comprising or consisting of the amino acid sequence of SEQ ID NO: 45;
t) a VH comprising or consisting of the amino acid sequence of SEQ ID NO: 21 and a VL comprising or consisting of the amino acid sequence of SEQ ID NO: 46;
u) a VH comprising or consisting of the amino acid sequence of SEQ ID NO: 22 and a VL comprising or consisting of the amino acid sequence of SEQ ID NO: 47;
v) a VH comprising or consisting of the amino acid sequence of SEQ ID NO: 23 and a VL comprising or consisting of the amino acid sequence of SEQ ID NO: 48;
w) a VH comprising or consisting of the amino acid sequence of SEQ ID NO: 24 and a VL comprising or consisting of the amino acid sequence of SEQ ID NO: 49; or
x) a VH comprising or consisting of the amino acid sequence of SEQ ID NO: 25 and a VL comprising or consisting of the amino acid sequence of SEQ ID NO: 50.

Certain amino acid substitutions may be made to provide one or more variant antibodies as described herein.

In any embodiments relating to Fab regions having defined VH and/or VL sequences (for example any VH and/or VL sequences defined as having certain percent identities and/or substitutions), preferably the VH sequence is not SEQ ID NO: 1 and the VL sequence is not SEQ ID NO: 26.

Other Antibodies Comprising Fab Regions Derived from ADT1-4

In one embodiment multispecific antibodies are provided, the anti-TCR delta variable 1 (anti-Vδ1) binding Fab region comprising:
a) an HCDR1 sequence comprising or consisting of the sequence of GDSVSSKSXIA (SEQ ID NO: 158);
b) an HCDR2 sequence comprising or consisting of the sequence of SEQ ID NO: 53
c) an HCDR3 sequence comprising or consisting of the sequence of X₂WX₃X₄X₅X₆DX₇ (SEQ ID NO: 162), wherein the HCDR3 sequence is not SEQ ID NO: 54;
d) an LCDR1 sequence comprising or consisting of the sequence of SEQ ID NO: 79;
e) an LCDR2 sequence comprising or consisting of the sequence of SEQ ID NO: 80; and
f) an LCDR3 sequence comprising or consisting of the sequence of QQX₈YX₉X₁₀X₁₁X₁₂X₁₃T (SEQ ID NO: 166), wherein the LCDR3 sequence is not SEQ ID NO: 81;
   and wherein each of X₁ to X₁₃ is a naturally occurring amino acid.

In one embodiment multispecific antibodies are provided, the anti-TCR delta variable 1 (anti-Vδ1) binding Fab region comprising:
a) an HCDR1 sequence comprising or consisting of the sequence of GDSVSSKSXIA (SEQ ID NO: 159);
b) an HCDR2 sequence comprising or consisting of the sequence of SEQ ID NO: 53
c) an HCDR3 sequence comprising or consisting of the sequence of X₂WX₃X₄X₅X₆DX₇ (SEQ ID NO: 163), wherein the HCDR3 sequence is not SEQ ID NO: 54;
d) an LCDR1 sequence comprising or consisting of the sequence of SEQ ID NO: 79;
e) an LCDR2 sequence comprising or consisting of the sequence of SEQ ID NO: 80; and
f) an LCDR3 sequence comprising or consisting of the sequence of QQX₈YX₉X₁₀X₁₁X₁₂X₁₃T (SEQ ID NO: 167), wherein the LCDR3 sequence is not SEQ ID NO: 81;
   and wherein: X₁ is selected from the group consisting of A and V; X₂ is selected from the group consisting of S and T; X₃ is selected from the group consisting of V, A and L; X₄ is selected from the group consisting of G, E, and D; X₅ is selected from the group consisting of Y and N; X₆ is selected from the group consisting of V, A and P; X₇ is selected from the group consisting of V, Y and R; X₈ is selected from the group consisting of K, R and G; X₉ is selected from the group consisting of S and K; X₁₀ is selected from the group consisting of T, Q, A, E and D; X₁₁ is selected from the group consisting of P, H and D; X₁₂ is selected from the group consisting of Q, R, K, W, P, E and I; and X₁₃ is selected from the group consisting of I, V and L. The antibody may further comprise an HFR1 sequence comprising the sequence of SEQ ID NO: 170 or 171; an HFR2 sequence comprising the sequence of SEQ ID NO: 172; an HFR3 sequence comprising the sequence of SEQ ID NO: 173; an HFR4 sequence comprising the sequence of SEQ ID NO: 174; an LFR1 sequence comprising the sequence of SEQ ID NO: 175; an LFR2 sequence comprising the sequence of SEQ ID NO: 176; an LFR3 sequence comprising the sequence of SEQ ID NO: 177 or 178; and an LFR4 sequence comprising the sequence of SEQ ID NO: 179, 180, 181 or 182.

In one embodiment multispecific antibodies are provided, the anti-TCR delta variable 1 (anti-Vδ1) binding Fab region comprising:
a) an HCDR1 sequence comprising or consisting of the sequence of GDSVSSKSX₁A (SEQ ID NO: 160);
b) an HCDR2 sequence comprising or consisting of the sequence of SEQ ID NO: 53
c) an HCDR3 sequence comprising or consisting of the sequence of X₂WX₃X₄X₅X₆DX₇ (SEQ ID NO: 164), wherein the HCDR3 sequence is not SEQ ID NO: 54;
d) an LCDR1 sequence comprising or consisting of the sequence of SEQ ID NO: 79;
e) an LCDR2 sequence comprising or consisting of the sequence of SEQ ID NO: 80; and
f) an LCDR3 sequence comprising or consisting of the sequence of QQX₈YX₉X₁₀X₁₁X₁₂X₁₃T (SEQ ID NO: 168), wherein the LCDR3 sequence is not SEQ ID NO: 81;
   and wherein X₁ is selected from the group consisting of A and V; X₂ is selected from the group consisting of S and T; X₃ is selected from the group consisting of V, A and L; X₄ is selected from the group consisting of G, E, and D; X₅ is selected from the group consisting of Y and N; X₆ is selected from the group consisting of V, A and P; X₇ is selected from the group consisting of V, Y and R; X₈ is selected from the group consisting of K, R and G; X₉ is selected from the group consisting of S and K; X₁₀ is selected from the group consisting of T, Q, A, E and D; X₁₁ is selected from the group consisting of P and H; X₁₂ is selected from the group consisting of Q, R, K, W, P, E and I; and X₁₃ is selected from the group consisting of I, V and L. The antibody may further comprise an HFR1 sequence comprising the sequence of SEQ ID NO: 170 or 171, an HFR2 sequence comprising the sequence of SEQ ID NO: 172, an HFR3 sequence comprising the sequence of SEQ ID NO: 173, an HFR4 sequence comprising the sequence of SEQ ID NO: 174, an LFR1 sequence comprising the sequence of SEQ ID NO: 175, an LFR2 sequence comprising the sequence of SEQ ID NO: 176, an LFR3 sequence comprising the sequence of SEQ ID NO: 177 and an LFR4 sequence comprising the sequence of SEQ ID NO: 179, 180, 181 or 182.

In one embodiment multispecific antibodies are provided, the anti-TCR delta variable 1 (anti-Vδ1) binding Fab region comprising:
a) an HCDR1 sequence comprising or consisting of the sequence of GDSVSSKSX₁A (SEQ ID NO: 161);
b) an HCDR2 sequence comprising or consisting of the sequence of SEQ ID NO: 53
c) an HCDR3 sequence comprising or consisting of the sequence of X₂WX₃X₄X₅X₆DX₇ (SEQ ID NO: 165), wherein the HCDR3 sequence is not SEQ ID NO: 54;
d) an LCDR1 sequence comprising or consisting of the sequence of SEQ ID NO: 79;
e) an LCDR2 sequence comprising or consisting of the sequence of SEQ ID NO: 80; and
f) an LCDR3 sequence comprising or consisting of the sequence of QQX₈YX₉X₁₀X₁₁X₁₂X₁₃T (SEQ ID NO: 169), wherein the LCDR3 sequence is not SEQ ID NO: 81;
   and wherein X₁ is selected from the group consisting of A and V; X₂ is selected from the group consisting of S and T; X₃ is selected from the group consisting of V, A and L; X₄ is selected from the group consisting of G and D; X₅ is selected from the group consisting of Y and N; X₆ is selected from the group consisting of V, A and P; X₇ is selected from the group consisting of V, Y and R; X₈ is selected from the group consisting of K and R; X₉ is selected from the group consisting of S and K; X₁₀ is selected from the group consisting of T, Q, A and E; X₁₁ is selected from the group consisting of P and H; X₁₂ is selected from the group consisting of Q, K, W, P and I; and X₁₃ is selected from the group consisting of V and L. The antibody may further comprise an HFR1 sequence comprising the sequence of SEQ ID NO: 170 or 171; an HFR2 sequence comprising the sequence of SEQ ID NO: 172; an HFR3 sequence comprising the sequence of SEQ ID NO: 173; an HFR4 sequence comprising the sequence of SEQ ID NO: 174; an LFR1 sequence comprising the sequence of SEQ ID NO: 175; an LFR2 sequence comprising the sequence of SEQ ID NO: 176; an LFR3 sequence comprising the sequence of SEQ ID NO: 177; and an LFR4 sequence comprising the sequence of SEQ ID NO: 179 or 181.

### ADT1-7-Derived Fab Regions

The present disclosure provides multispecific antibodies comprising Fab regions derived from parental antibody ADT1-7 (having a variable heavy region sequence according to SEQ ID NO: 106 and a variable light region sequence according to SEQ ID No: 118), for example as set out in the following. ADT1-7 is also referred to herein as E07, and ADT1-7 and E07 are used interchangeably.

Antibodies Comprising Particular CDR Sequences Derived from ADT1-7

The multispecific antibodies provided herein include the following Fab regions having particular sequences derived from ADT1-7.

For example, in some embodiments, multispecific antibodies are provided, the anti-TCR delta variable 1 (anti-Vδ1) binding Fab region comprising a heavy chain variable region comprising a VHCDR3 comprising or consisting of an amino acid sequence selected from the group consisting of SEQ ID NOs: 133 to 143; and/or a light chain variable region comprising a VLCDR3 comprising or consisting of an amino acid sequence selected from the group consisting of SEQ ID NOs: 147 to 157. Certain amino acid substitutions may be made to provide one or more variant antibodies as described herein.

In some embodiments multispecific antibodies are provided, the anti-TCR delta variable 1 (anti-Vδ1) binding Fab region comprising a heavy chain variable region comprising a VHCDR3 comprising or consisting of an amino acid sequence selected from the group consisting of SEQ ID NOs: 138, 142 and 143 and/or a light chain variable region comprising a VLCDR3 comprising or consisting of an amino acid sequence selected from the group consisting of SEQ ID NOs: 152, 156 and 157, Certain amino acid substitutions may be made to provide one or more variant antibodies as described herein.

There is also provided multispecific antibodies, the anti-TCR delta variable 1 (anti-Vδ1) binding Fab region comprising:
a heavy chain variable region comprising:
   a VHCDR1 comprising or consisting of the amino acid sequence of SEQ ID NO: 130;
   a VHCDR2 comprising or consisting of the amino acid sequence of SEQ ID NO: 131; and
   a VHCDR3 comprising or consisting of an amino acid sequence selected from the group consisting of: SEQ ID NOs: 133 to 143; and
a light chain variable region comprising:
   a VLCDR1 comprising or consisting of the amino acid sequence of SEQ ID NO: 144;
   a VLCDR2 comprising or consisting of the amino acid sequence of SEQ ID NO: 145; and
   a VLCDR3 comprising or consisting of an amino acid sequence selected from the group consisting of: SEQ ID NOs: 147 to 157.

Certain amino acid substitutions may be made to provide one or more variant antibodies as described herein.

Also provided are multispecific antibodies, the anti-TCR delta variable 1 (anti-Vδ1) binding Fab region comprising:
a heavy chain variable region comprising:
   a VHCDR1 comprising or consisting of the amino acid sequence of SEQ ID NO: 130;
   a VHCDR2 comprising or consisting of the amino acid sequence of SEQ ID NO: 131; and
   a VHCDR3 comprising or consisting of an amino acid sequence selected from the group consisting of: SEQ ID NOs: 138, 142 and 143; and
a light chain variable region comprising:
   a VLCDR1 comprising or consisting of the amino acid sequence of SEQ ID NO: 144;
   a VLCDR2 comprising or consisting of the amino acid sequence of SEQ ID NO: 145; and
   a VLCDR3 comprising or consisting of an amino acid sequence selected from the group consisting of: SEQ ID NOs: 152, 156 and 157.

Certain amino acid substitutions may be made to provide one or more variant antibodies as described herein.

Further embodiments are provided below.

### ADT1-7-10 and Fragments and Variants Thereof

Certain embodiments relate to the antibody ADT1-7-10 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 133 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 147. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 133 and/or a light chain variable region comprising a VLCDR3 comprising an amino acid sequence having at least 90% identity to the amino acid sequence of SEQ ID NO: 147. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 133 and/or a light chain variable region comprising a VLCDR3 comprising the amino acid sequence of SEQ ID NO: 147.

Amino acid substitutions may be made to provide variant antibodies having Fab regions derived from ADT1-7-10, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 133, optionally comprising 1 or 2 amino acid substitutions, and/or a light chain variable region comprising a VLCDR3 comprising the amino acid sequence of SEQ ID NO: 147, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 133; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 147.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 133; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 147.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 133; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 147.

Amino acid substitutions may be made to provide variant antibodies having Fab regions derived from ADT1-7-10, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 133 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 147 optionally comprising 1 or 2 amino acid substitutions. The amino acid substitutions may be conservative amino acid substitutions.

The antibodies may alternatively consist of the specified sequences (with or without amino acid substitutions).

### ADT1-7-15 and Fragments and Variants Thereof

Certain embodiments relate to the antibody ADT1-7-15 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 134 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 148. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 134 and/or a light chain variable region comprising a VLCDR3 comprising an amino acid sequence having at least 90% identity to the amino acid sequence of SEQ ID NO: 148. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 134 and/or a light chain variable region comprising a VLCDR3 comprising the amino acid sequence of SEQ ID NO: 148.

Amino acid substitutions may be made to provide variant antibodies having Fab regions derived from ADT1-7-15, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 134, optionally comprising 1 or 2 amino acid substitutions, and/or a light chain variable region comprising a VLCDR3 comprising the amino acid sequence of SEQ ID NO: 148, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 134; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 148.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 134; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 148.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 134; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 148.

Amino acid substitutions may be made to provide variant antibodies having Fab regions derived from ADT1-7-15, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 134 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 148 optionally comprising 1 or 2 amino acid substitutions. The amino acid substitutions may be conservative amino acid substitutions.

The antibodies may alternatively consist of the specified sequences (with or without amino acid substitutions).

### ADT1-7-17 and Fragments and Variants Thereof

Certain embodiments relate to the antibody ADT1-7-17 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 135 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 149. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 135 and/or a light chain variable region comprising a VLCDR3 comprising an amino acid sequence having at least 90% identity to the amino acid sequence of SEQ ID NO: 149. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 135 and/or a light chain variable region comprising a VLCDR3 comprising the amino acid sequence of SEQ ID NO: 149.

Amino acid substitutions may be made to provide variant antibodies having Fab regions derived from ADT1-7-17, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 135, optionally comprising 1 or 2 amino acid substitutions, and/or a light chain variable region comprising a VLCDR3 comprising the amino acid sequence of SEQ ID NO: 149, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 135; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 149.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 135; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 149.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 135; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 149.

Amino acid substitutions may be made to provide variant antibodies having Fab regions derived from ADT1-7-17, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 135 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 149 optionally comprising 1 or 2 amino acid substitutions. The amino acid substitutions may be conservative amino acid substitutions.

The antibodies may alternatively consist of the specified sequences (with or without amino acid substitutions).

### ADT1-7-18 and Fragments and Variants Thereof

Certain embodiments relate to the antibody ADT1-7-18 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 136 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 150. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 136 and/or a light chain variable region comprising a VLCDR3 comprising an amino acid sequence having at least 90% identity to the amino acid sequence of SEQ ID NO: 150. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 136 and/or a light chain variable region comprising a VLCDR3 comprising the amino acid sequence of SEQ ID NO: 150.

Amino acid substitutions may be made to provide variant antibodies having Fab regions derived from ADT1-7-18, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 136, optionally comprising 1 or 2 amino acid substitutions, and/or a light chain variable region comprising a VLCDR3 comprising the amino acid sequence of SEQ ID NO: 150, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 136; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 150.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 136; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 150.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 136; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 150.

Amino acid substitutions may be made to provide variant antibodies having Fab regions derived from ADT1-7-18, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 136 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 150 optionally comprising 1 or 2 amino acid substitutions. The amino acid substitutions may be conservative amino acid substitutions.

The antibodies may alternatively consist of the specified sequences (with or without amino acid substitutions).

### ADT1-7-19 and Fragments and Variants Thereof

Certain embodiments relate to the antibody ADT1-7-19 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 137 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 151. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 137 and/or a light chain variable region comprising a VLCDR3 comprising an amino acid sequence having at least 90% identity to the amino acid sequence of SEQ ID NO: 151. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 137 and/or a light chain variable region comprising a VLCDR3 comprising the amino acid sequence of SEQ ID NO: 151.

Amino acid substitutions may be made to provide variant antibodies having Fab regions derived from ADT1-7-19, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 137, optionally comprising 1 or 2 amino acid substitutions, and/or a light chain variable region comprising a VLCDR3 comprising the amino acid sequence of SEQ ID NO: 151, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 137; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 151.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 137; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 151.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 137; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 151.

Amino acid substitutions may be made to provide variant antibodies having Fab regions derived from ADT1-7-19, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 137 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 151 optionally comprising 1 or 2 amino acid substitutions. The amino acid substitutions may be conservative amino acid substitutions.

The antibodies may alternatively consist of the specified sequences (with or without amino acid substitutions).

### ADT1-7-20 and Fragments and Variants Thereof

Certain embodiments relate to the antibody ADT1-7-20 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 138 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 152. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 138 and/or a light chain variable region comprising a VLCDR3 comprising an amino acid sequence having at least 90% identity to the amino acid sequence of SEQ ID NO: 152. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 138 and/or a light chain variable region comprising a VLCDR3 comprising the amino acid sequence of SEQ ID NO: 152.

Amino acid substitutions may be made to provide variant antibodies having Fab regions derived from ADT1-7-20, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 138, optionally comprising 1 or 2 amino acid substitutions, and/or a light chain variable region comprising a VLCDR3 comprising the amino acid sequence of SEQ ID NO: 152, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 138; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 152.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 138; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 152.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 138; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 152.

Amino acid substitutions may be made to provide variant antibodies having Fab regions derived from ADT1-7-20, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 138 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 152 optionally comprising 1 or 2 amino acid substitutions. The amino acid substitutions may be conservative amino acid substitutions.

The antibodies may alternatively consist of the specified sequences (with or without amino acid substitutions).

### ADT1-7-22 and Fragments and Variants Thereof

Certain embodiments relate to the antibody ADT1-7-22 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 139 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 153. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 139 and/or a light chain variable region comprising a VLCDR3 comprising an amino acid sequence having at least 90% identity to the amino acid sequence of SEQ ID NO: 153. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 139 and/or a light chain variable region comprising a VLCDR3 comprising the amino acid sequence of SEQ ID NO: 153.

Amino acid substitutions may be made to provide variant antibodies having Fab regions derived from ADT1-7-22, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 139, optionally comprising 1 or 2 amino acid substitutions, and/or a light chain variable region comprising a VLCDR3 comprising the amino acid sequence of SEQ ID NO: 153, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 139; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 153.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 139; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 153.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 139; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 153.

Amino acid substitutions may be made to provide variant antibodies having Fab regions derived from ADT1-7-22, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 139 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 153 optionally comprising 1 or 2 amino acid substitutions. The amino acid substitutions may be conservative amino acid substitutions.

The antibodies may alternatively consist of the specified sequences (with or without amino acid substitutions).

### ADT1-7-23 and Fragments and Variants Thereof

Certain embodiments relate to the antibody ADT1-7-23 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 140 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 154. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 140 and/or a light chain variable region comprising a VLCDR3 comprising an amino acid sequence having at least 90% identity to the amino acid sequence of SEQ ID NO: 154. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 140 and/or a light chain variable region comprising a VLCDR3 comprising the amino acid sequence of SEQ ID NO: 154.

Amino acid substitutions may be made to provide variant antibodies having Fab regions derived from ADT1-7-23, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 140, optionally comprising 1 or 2 amino acid substitutions, and/or a light chain variable region comprising a VLCDR3 comprising the amino acid sequence of SEQ ID NO: 154, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 140; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 154.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 140; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 154.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 140; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 154.

Amino acid substitutions may be made to provide variant antibodies having Fab regions derived from ADT1-7-23, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 140 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 154 optionally comprising 1 or 2 amino acid substitutions. The amino acid substitutions may be conservative amino acid substitutions.

The antibodies may alternatively consist of the specified sequences (with or without amino acid substitutions).

### ADT1-7-42 and Fragments and Variants Thereof

Certain embodiments relate to the antibody ADT1-7-42 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 141 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 155. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 141 and/or a light chain variable region comprising a VLCDR3 comprising an amino acid sequence having at least 90% identity to the amino acid sequence of SEQ ID NO: 155. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 141 and/or a light chain variable region comprising a VLCDR3 comprising the amino acid sequence of SEQ ID NO: 155.

Amino acid substitutions may be made to provide variant antibodies having Fab regions derived from ADT1-7-42, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 141, optionally comprising 1 or 2 amino acid substitutions, and/or a light chain variable region comprising a VLCDR3 comprising the amino acid sequence of SEQ ID NO: 155, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 141; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 155.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 141; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 155.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 141; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 155.

Amino acid substitutions may be made to provide variant antibodies having Fab regions derived from ADT1-7-42, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 141 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 155 optionally comprising 1 or 2 amino acid substitutions. The amino acid substitutions may be conservative amino acid substitutions.

The antibodies may alternatively consist of the specified sequences (with or without amino acid substitutions).

### ADT1-7-3 and Fragments and Variants Thereof

Certain embodiments relate to the antibody ADT1-7-3 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 142 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 156. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 142 and/or a light chain variable region comprising a VLCDR3 comprising an amino acid sequence having at least 90% identity to the amino acid sequence of SEQ ID NO: 156. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 142 and/or a light chain variable region comprising a VLCDR3 comprising the amino acid sequence of SEQ ID NO: 156.

Amino acid substitutions may be made to provide variant antibodies having Fab regions derived from ADT1-7-3, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 142, optionally comprising 1 or 2 amino acid substitutions, and/or a light chain variable region comprising a VLCDR3 comprising the amino acid sequence of SEQ ID NO: 156, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 142; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 156.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 142; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 156.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 142; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 156.

Amino acid substitutions may be made to provide variant antibodies having Fab regions derived from ADT1-7-3, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 142 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 156 optionally comprising 1 or 2 amino acid substitutions. The amino acid substitutions may be conservative amino acid substitutions.

The antibodies may alternatively consist of the specified sequences (with or without amino acid substitutions).

### ADT1-7-61 and Fragments and Variants Thereof

Certain embodiments relate to the antibody ADT1-7-61 and fragments and variants thereof.

For example, in some embodiments, multispecific antibodies are provided, the anti-TCR delta variable 1 (anti-Vδ1) binding Fab region comprising a heavy chain variable region comprising a VHCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 143 and/or a light chain variable region comprising a VLCDR3 comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 157. In one embodiment, an antibody or antigen-binding-fragment or variant thereof is provided comprising a heavy chain variable region comprising a VHCDR3 comprising an amino acid sequence having at least 90% identity to the amino acid sequence of SEQ ID NO: 143 and/or a light chain variable region comprising a VLCDR3 comprising an amino acid sequence having at least 90% identity to the amino acid sequence of SEQ ID NO: 157. In one embodiment, multispecific antibodies are provided, the anti-TCR delta variable 1 (anti-Vδ1) binding Fab region comprising a heavy chain variable region comprising a VHCDR3 comprising the amino acid sequence of SEQ ID NO: 143 and/or a light chain variable region comprising a VLCDR3 comprising the amino acid sequence of SEQ ID NO: 157.

Amino acid substitutions may be made to provide variant antibodies having Fab regions derived from ADT1-7-61, for example multispecific antibodies are provided, the anti-TCR delta variable 1 (anti-Vδ1) binding Fab region comprising a heavy chain variable region comprising a VHCDR3 comprising the amino acid sequence of SEQ ID NO: 143, optionally comprising 1 or 2 amino acid substitutions, and/or a light chain variable region comprising a VLCDR3 comprising the amino acid sequence of SEQ ID NO: 157, optionally comprising 1 or 2 amino acid substitutions. The amino acid substitutions may be conservative amino acid substitutions.

In some embodiments, multispecific antibodies are provided, the anti-TCR delta variable 1 (anti-Vδ1) binding Fab region comprising:
a heavy chain variable region comprising a VHCDR1 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 130, a VHCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 131 and a VHCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 143; and/or
a light chain variable region comprising a VLCDR1 comprising the at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to amino acid sequence of SEQ ID NO: 144, a VLCDR2 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 145 and a VLCDR3 comprising at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 157.

In one embodiment, multispecific antibodies are provided, the anti-TCR delta variable 1 (anti-Vδ1) binding Fab region comprising:
a heavy chain variable region comprising a VHCDR1 comprising at least 90% identity to the amino acid sequence of SEQ ID NO: 130, a VHCDR2 comprising at least 90% identity to the amino acid sequence of SEQ ID NO: 131 and a VHCDR3 comprising at least 90% identity to the amino acid sequence of SEQ ID NO: 143; and/or
a light chain variable region comprising a VLCDR1 comprising the at least 90% identity to the amino acid sequence of SEQ ID NO: 144, a VLCDR2 comprising at least 90% identity to the amino acid sequence of SEQ ID NO: 145 and a VLCDR3 comprising at least 90% identity to the amino acid sequence of SEQ ID NO: 157.

In one embodiment, multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130, a VHCDR2 comprising the amino acid sequence of SEQ ID NO: 131 and a VHCDR3 comprising the amino acid sequence of SEQ ID NO: 143; and
a light chain variable region comprising a VLCDR1 comprising the amino acid sequence of SEQ ID NO: 144, a VLCDR2 comprising the amino acid sequence of SEQ ID NO: 145 and a VLCDR3 comprising the amino acid sequence of SEQ ID NO: 157.

Amino acid substitutions may be made to provide variant antibodies having Fab regions derived from ADT1-7-61, for example multispecific antibodies are provided, the anti-TCR delta variable 1 (anti-Vδ1) binding Fab region comprising:
a heavy chain variable region comprising a VHCDR1 comprising the amino acid sequence of SEQ ID NO: 130 optionally comprising 1 or 2 amino acid substitutions, a VHCDR2 comprising the amino acid sequence of SEQ ID NO: 131 optionally comprising 1 or 2 amino acid substitutions, and a VHCDR3 comprising the amino acid sequence of SEQ ID NO: 143 optionally comprising 1 or 2 amino acid substitutions; and/or
a light chain variable region comprising a VLCDR1 comprising the amino acid sequence of SEQ ID NO: 144 optionally comprising 1 or 2 amino acid substitutions, a VLCDR2 comprising the amino acid sequence of SEQ ID NO: 145 optionally comprising 1 or 2 amino acid substitutions and a VLCDR3 comprising the amino acid sequence of SEQ ID NO: 157 optionally comprising 1 or 2 amino acid substitutions. The amino acid substitutions may be conservative amino acid substitutions.

The antibodies may alternatively consist of the specified sequences (with or without amino acid substitutions).

Antibodies Comprising Heavy and/or Light Chain Variable Regions Derived from ADT1-7

Provided herein are multispecific antibodies, the anti-TCR delta variable 1 (anti-Vδ1) binding Fab region comprising:
a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 107 to 117; and/or
a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 119 to 129.

Also provided are multispecific antibodies, the anti-TCR delta variable 1 (anti-Vδ1) binding Fab region comprising:
a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 112, 116 and 117; and/or
a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 124, 128 and 129.

Also provided are multispecific antibodies, the anti-TCR delta variable 1 (anti-Vδ1) binding Fab region comprising:
a heavy chain variable region comprising or consisting of a sequence having at least 90% identity to a sequence selected from the group consisting of SEQ ID NOs: 107 to 117; and/or
a light chain variable region comprising or consisting of a sequence having at least 90% identity to a sequence selected from the group consisting of SEQ ID NOs: 119 to 129.

Also provided are multispecific antibodies, the anti-TCR delta variable 1 (anti-Vδ1) binding Fab region comprising:
a heavy chain variable region comprising or consisting of a sequence having at least 90% identity to a sequence selected from the group consisting of SEQ ID NOs: 112, 116 and 117; and/or
a light chain variable region comprising or consisting of a sequence having at least 90% identity to a sequence selected from the group consisting of SEQ ID NOs: 124, 128 and 129.

Optionally the antibodies above retain the corresponding CDR sequences such that any variability in the VH and VL sequences occurs in the framework regions.

Provided herein are multispecific antibodies, the anti-TCR delta variable 1 (anti-Vδ1) binding Fab region comprising:
a heavy chain variable region comprising or consisting of a sequence selected from the group consisting of SEQ ID NOs: 107 to 117; and/or
a light chain variable region comprising or consisting of a sequence selected from the group consisting of SEQ ID NOs: 119 to 129.

Also provided herein are multispecific antibodies, the anti-TCR delta variable 1 (anti-Vδ1) binding Fab region comprising:
a heavy chain variable region comprising or consisting of a sequence selected from the group consisting of SEQ ID NOs: 112, 116 and 117; and/or
a light chain variable region comprising or consisting of a sequence selected from the group consisting of SEQ ID NOs: 124, 128 and 129.

In some embodiments, multispecific antibodies are provided, the anti-TCR delta variable 1 (anti-Vδ1) binding Fab region comprising:
a) a VH comprising or consisting of an amino acid sequence that is at least 90% identical to the amino acid sequence of SEQ ID NO: 107 and a VL comprising or consisting of an amino acid sequence that is at least 90% identical to the amino acid sequence of SEQ ID NO: 119;
b) a VH comprising or consisting of an amino acid sequence that is at least 90% identical to the amino acid sequence of SEQ ID NO: 108 and a VL comprising or consisting of an amino acid sequence that is at least 90% identical to the amino acid sequence of SEQ ID NO: 120;
c) a VH comprising or consisting of an amino acid sequence that is at least 90% identical to the amino acid sequence of SEQ ID NO: 109 and a VL comprising or consisting of an amino acid sequence that is at least 90% identical to the amino acid sequence of SEQ ID NO: 121;
d) a VH comprising or consisting of an amino acid sequence that is at least 90% identical to the amino acid sequence of SEQ ID NO: 110 and a VL comprising or consisting of an amino acid sequence that is at least 90% identical to the amino acid sequence of SEQ ID NO: 122;
e) a VH comprising or consisting of an amino acid sequence that is at least 90% identical to the amino acid sequence of SEQ ID NO: 111 and a VL comprising or consisting of an amino acid sequence that is at least 90% identical to the amino acid sequence of SEQ ID NO: 123;
f) a VH comprising or consisting of an amino acid sequence that is at least 90% identical to the amino acid sequence of SEQ ID NO: 112 and a VL comprising or consisting of an amino acid sequence that is at least 90% identical to the amino acid sequence of SEQ ID NO: 124;
g) a VH comprising or consisting of an amino acid sequence that is at least 90% identical to the amino acid sequence of SEQ ID NO: 113 and a VL comprising or consisting of an amino acid sequence that is at least 90% identical to the amino acid sequence of SEQ ID NO: 125;
h) a VH comprising or consisting of an amino acid sequence that is at least 90% identical to the amino acid sequence of SEQ ID NO: 114 and a VL comprising or consisting of an amino acid sequence that is at least 90% identical to the amino acid sequence of SEQ ID NO: 126;
i) a VH comprising or consisting of an amino acid sequence that is at least 90% identical to the amino acid sequence of SEQ ID NO: 115 and a VL comprising or consisting of an amino acid sequence that is at least 90% identical to the amino acid sequence of SEQ ID NO: 127;
j) a VH comprising or consisting of an amino acid sequence that is at least 90% identical to the amino acid sequence of SEQ ID NO: 116 and a VL comprising or consisting of an amino acid sequence that is at least 90% identical to the amino acid sequence of SEQ ID NO: 128; or
k) a VH comprising or consisting of an amino acid sequence that is at least 90% identical to the amino acid sequence of SEQ ID NO: 117 and a VL comprising or consisting of an amino acid sequence that is at least 90% identical to the amino acid sequence of SEQ ID NO: 129.

Optionally the antibodies above retain the corresponding CDR sequences such that any variability in the VH and VL sequences occurs in the framework regions.

In some embodiments, multispecific antibodies are provided, the anti-TCR delta variable 1 (anti-Vδ1) binding Fab region comprising:
a) a VH comprising or consisting of the amino acid sequence of SEQ ID NO: 107 and a VL comprising or consisting of the amino acid sequence of SEQ ID NO: 119 optionally comprising up to 5, up to 4, up to 3, up to 2 or 1 amino acid substitutions across both variable regions;
b) a VH comprising or consisting of the amino acid sequence of SEQ ID NO: 108 and a VL comprising or consisting of the amino acid sequence of SEQ ID NO: 120 optionally comprising up to 5, up to 4, up to 3, up to 2 or 1 amino acid substitutions across both variable regions;
c) a VH comprising or consisting of the amino acid sequence of SEQ ID NO: 109 and a VL comprising or consisting of the amino acid sequence of SEQ ID NO: 121 optionally comprising up to 5, up to 4, up to 3, up to 2 or 1 amino acid substitutions across both variable regions;
d) a VH comprising or consisting of the amino acid sequence of SEQ ID NO: 110 and a VL comprising or consisting of the amino acid sequence of SEQ ID NO: 122 optionally comprising up to 5, up to 4, up to 3, up to 2 or 1 amino acid substitutions across both variable regions;
e) a VH comprising or consisting of the amino acid sequence of SEQ ID NO: 111 and a VL comprising or consisting of the amino acid sequence of SEQ ID NO: 123 optionally comprising up to 5, up to 4, up to 3, up to 2 or 1 amino acid substitutions across both variable regions;
f) a VH comprising or consisting of the amino acid sequence of SEQ ID NO: 112 and a VL comprising or consisting of the amino acid sequence of SEQ ID NO: 124 optionally comprising up to 5, up to 4, up to 3, up to 2 or 1 amino acid substitutions across both variable regions;
g) a VH comprising or consisting of the amino acid sequence of SEQ ID NO: 113 and a VL comprising or consisting of the amino acid sequence of SEQ ID NO: 125 optionally comprising up to 5, up to 4, up to 3, up to 2 or 1 amino acid substitutions across both variable regions;
h) a VH comprising or consisting of the amino acid sequence of SEQ ID NO: 114 and a VL comprising or consisting of the amino acid sequence of SEQ ID NO: 126 optionally comprising up to 5, up to 4, up to 3, up to 2 or 1 amino acid substitutions across both variable regions;
i) a VH comprising or consisting of the amino acid sequence of SEQ ID NO: 115 and a VL comprising or consisting of the amino acid sequence of SEQ ID NO: 127 optionally comprising up to 5, up to 4, up to 3, up to 2 or 1 amino acid substitutions across both variable regions;
j) a VH comprising or consisting of the amino acid sequence of SEQ ID NO: 116 and a VL comprising or consisting of the amino acid sequence of SEQ ID NO: 128 optionally comprising up to 5, up to 4, up to 3, up to 2 or 1 amino acid substitutions across both variable regions; or
k) a VH comprising or consisting of the amino acid sequence of SEQ ID NO: 117 and a VL comprising or consisting of the amino acid sequence of SEQ ID NO: 129 optionally comprising up to 5, up to 4, up to 3, up to 2 or 1 amino acid substitutions across both variable regions.

“Across both variable regions” means collectively, the antibody may comprise up to the specified number of substitutions in total when considering both the heavy and light chain variable regions. The amino acid substitutions may be conservative amino acid substitutions. In some embodiments, the substitutions (if present) may occur anywhere in the variable region sequences. In preferred embodiments, the substitutions (if present) may be limited to the framework regions. Accordingly in some embodiments, the amino acid substitutions do not occur in a CDR sequence.

Optionally the antibodies above retain the corresponding CDR sequences such that any variability in the VH and VL sequences occurs in the framework regions.

In some embodiments, multispecific antibodies are provided, the anti-TCR delta variable 1 (anti-Vδ1) binding Fab region comprising:
a) a VH comprising or consisting of the amino acid sequence of SEQ ID NO: 107 and a VL comprising or consisting of the amino acid sequence of SEQ ID NO: 119;
b) a VH comprising or consisting of the amino acid sequence of SEQ ID NO: 108 and a VL comprising or consisting of the amino acid sequence of SEQ ID NO: 120;
c) a VH comprising or consisting of the amino acid sequence of SEQ ID NO: 109 and a VL comprising or consisting of the amino acid sequence of SEQ ID NO: 121;
d) a VH comprising or consisting of the amino acid sequence of SEQ ID NO: 110 and a VL comprising or consisting of the amino acid sequence of SEQ ID NO: 122;
e) a VH comprising or consisting of the amino acid sequence of SEQ ID NO: 111 and a VL comprising or consisting of the amino acid sequence of SEQ ID NO: 123;
f) a VH comprising or consisting of the amino acid sequence of SEQ ID NO: 112 and a VL comprising or consisting of the amino acid sequence of SEQ ID NO: 124;
g) a VH comprising or consisting of the amino acid sequence of SEQ ID NO: 113 and a VL comprising or consisting of the amino acid sequence of SEQ ID NO: 125;
h) a VH comprising or consisting of the amino acid sequence of SEQ ID NO: 114 and a VL comprising or consisting of the amino acid sequence of SEQ ID NO: 126;
i) a VH comprising or consisting of the amino acid sequence of SEQ ID NO: 115 and a VL comprising or consisting of the amino acid sequence of SEQ ID NO: 127;
j) a VH comprising or consisting of the amino acid sequence of SEQ ID NO: 116 and a VL comprising or consisting of the amino acid sequence of SEQ ID NO: 128; or
k) a VH comprising or consisting of the amino acid sequence of SEQ ID NO: 117 and a VL comprising or consisting of the amino acid sequence of SEQ ID NO: 129.

Certain amino acid substitutions may be made to provide one or more variant antibodies as described herein.

In any embodiments relating to Fab regions having defined VH and/or VL sequences (for example any VH and/or VL sequences defined as having certain percent identities and/or substitutions), preferably the VH sequence is not SEQ ID NO: 106 and the VL sequence is not SEQ ID NO: 118.

Other Antibodies Comprising Fab Regions Derived from ADT1-7

In one embodiment multispecific antibodies are provided, the anti-TCR delta variable 1 (anti-Vδ1) binding Fab region comprising:
a) an HCDR1 sequence comprising or consisting of the sequence of SEQ ID NO: 130;
b) an HCDR2 sequence comprising or consisting of the sequence of SEQ ID NO: 131;
c) an HCDR3 sequence comprising or consisting of the sequence of X₁X₂YX₃X₄AFDI (SEQ ID NO: 183), wherein the HCDR3 sequence is not SEQ ID NO: 132;
d) an LCDR1 sequence comprising or consisting of the sequence of SEQ ID NO: 144;
e) an LCDR2 sequence comprising or consisting of the sequence of SEQ ID NO: 145; and
f) an LCDR3 sequence comprising or consisting of the sequence of QQX₅X₆X₇X₈LX₉T (SEQ ID NO: 186), wherein the LCDR3 sequence is no SEQ ID NO: 146; wherein each of X₁ to X₉ is a naturally occurring amino acid.

In one embodiment multispecific antibodies are provided, the anti-TCR delta variable 1 (anti-Vδ1) binding Fab region comprising:
a) an HCDR1 sequence comprising or consisting of the sequence of SEQ ID NO: 130;
b) an HCDR2 sequence comprising or consisting of the sequence of SEQ ID NO: 131;
c) an HCDR3 sequence comprising or consisting of the sequence of X₁X₂YX₃X₄AFDI (SEQ ID NO: 184), wherein the HCDR3 sequence is not SEQ ID NO: 132;
d) an LCDR1 sequence comprising or consisting of the sequence of SEQ ID NO: 144;
e) an LCDR2 sequence comprising or consisting of the sequence of SEQ ID NO: 145; and
f) an LCDR3 sequence comprising or consisting of the sequence of QQX₅X₆X₇X₈LX₉T (SEQ ID NO: 187), wherein the LCDR3 sequence is no SEQ ID NO: 146;
   wherein X₁ is selected from the group consisting of D, I and V; X₂ is selected from the group consisting of D and S; X₃ is selected from the group consisting of N, E, D, Q and A; X₄ is selected from the group consisting of D and E; X₅ is selected from the group consisting of T and S; X₆ is selected from the group consisting of A, G, Y and S; X₇ is selected from the group consisting of S and D; X₈ is selected from the group consisting of T, E and G; and X₉ is selected from the group consisting of L and D. The antibody may further comprise an HFR1 sequence comprising the sequence of SEQ ID NO: 189; an HFR2 sequence comprising the sequence of SEQ ID NO: 190; an HFR3 sequence comprising the sequence of SEQ ID NO: 191; an HFR4 sequence comprising the sequence of SEQ ID NO: 192; an LFR1 sequence comprising the sequence of SEQ ID NO: 193 and 194; an LFR2 sequence comprising the sequence of SEQ ID NO: 195; an LFR3 sequence comprising the sequence of SEQ ID NO: 196; and an LFR4 sequence comprising the sequence of SEQ ID NO: 197.

In one embodiment multispecific antibodies are provided, the anti-TCR delta variable 1 (anti-Vδ1) binding Fab region comprising:

a) an HCDR1 sequence comprising or consisting of the sequence of SEQ ID NO: 130
b) an HCDR2 sequence comprising or consisting of the sequence of SEQ ID NO: 131
c) an HCDR3 sequence comprising or consisting of the sequence of X₁X₂YX₃X₄AFDI (SEQ ID NO: 185), wherein the HCDR3 sequence is not SEQ ID NO: 132;
d) an LCDR1 sequence comprising or consisting of the sequence of SEQ ID NO: 144;
e) an LCDR2 sequence comprising or consisting of the sequence of SEQ ID NO: 145; and
f) an LCDR3 sequence comprising or consisting of the sequence of QQX₅X₆X₇X₈LX₉T (SEQ ID NO: 188), wherein the LCDR3 sequence is no SEQ ID NO: 146;
   wherein X₁ is selected from the group consisting of D and V; X₂ is selected from the group consisting of D and S; X₃ is selected from the group consisting of D, Q and A; X₄ is selected from the group consisting of D and E; X₅ is S; X₆ is selected from the group consisting of A and Y; X₇ is S; X₈ is selected from the group consisting of E and G; and X₉ is selected from the group consisting of L and D. The antibody may further comprise an HFR1 sequence comprising the sequence of SEQ ID NO: 189; an HFR2 sequence comprising the sequence of SEQ ID NO: 190; an HFR3 sequence comprising the sequence of SEQ ID NO: 191; an HFR4 sequence comprising the sequence of SEQ ID NO: 192; an LFR1 sequence comprising the sequence of SEQ ID NO: 193; an LFR2 sequence comprising the sequence of SEQ ID NO: 195; an LFR3 sequence comprising the sequence of SEQ ID NO: 196; and an LFR4 sequence comprising the sequence of SEQ ID NO: 197.

### Antibodies Having Sequence Substitutions

The skilled person is aware that various amino acids have similar properties. One or more such amino acids of a substance can often be substituted by one or more other such amino acids without eliminating a desired activity of that substance.

Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

A “conservative” amino acid substitution is an amino acid substitution in which an amino acid residue is replaced with another amino acid residue of similar chemical structure, and which is expected to have little influence on the function, activity or other biological properties of the polypeptide. Such conservative substitutions suitably are substitutions in which one amino acid within the following groups is substituted by another amino acid residue from within the same group:

| Group | Amino acid residue |
|---|---|
| Non-polar aliphatic | Glycine |
| | Alanine |
| | Valine |
| | Methionine |
| | Leucine |
| | Isoleucine |
| Aromatic | Phenylalanine |
| | Tyrosine |
| | Tryptophan |
| Polar uncharged | Serine |
| | Threonine |
| | Cysteine |
| | Proline |
| | Asparagine |
| | Glutamine |
| Negatively charged | Aspartate |
| | Glutamate |
| Positively charged | Lysine |
| | Arginine |
| | Histidine |

Suitably, a hydrophobic amino acid residue is a non-polar amino acid. More suitably, a hydrophobic amino acid residue is selected from V, I, L, M, F, W or C. In some embodiments, a hydrophobic amino acid residue is selected from glycine, alanine, valine, methionine, leucine, isoleucine, phenylalanine, tyrosine, or tryptophan.

Therefore, references to “conservative” amino acid substitutions refer to amino acid substitutions in which one or more of the amino acids in the sequence of the antibody (e.g. in the CDRs or in the VH or VL sequences) is substituted with another amino acid in the same class as indicated above. Conservative amino acid substitutions may be preferred in the CDR regions to minimise adverse effects on the function of the antibody. However, conservative amino acid substitutions may also occur in the framework regions. Therefore, in some embodiments, any substitutions in the CDRs may be conservative substitutions, whereas substitutions in the framework regions may by substitutions of naturally occurring amino acids with another other naturally occurring amino acids.

Amino acid deletions or insertions can also be made relative to the amino acid sequences provided for the antibodies described herein. Thus, for example, amino acids which do not have a substantial effect on the activity of the polypeptide, or at least which do not eliminate such activity, can be deleted. Such deletions can be advantageous since the overall length and the molecular weight of a polypeptide can be reduced whilst still retaining activity. This can enable the amount of polypeptide required for a particular purpose to be reduced for example, dosage levels can be reduced.

Amino acid changes relative to the sequences provided herein can be made using any suitable technique e.g. by using site-directed mutagenesis or solid-state synthesis.

It should be appreciated that amino acid substitutions or insertions within the scope of the present disclosure can be made using naturally occurring or non-naturally occurring amino acids, although naturally occurring amino acids may be preferred. Whether or not natural or synthetic amino acids are used, it may be preferred that only L-amino acids are present.

Various embodiments comprising optional amino acid substitutions the provided sequences are provided herein. In addition, in one embodiment of the disclosure there is provided antibodies, or antigen-binding fragment thereof, of the disclosure comprising up to 10, suitably up to 5, or suitably up to 2 amino acid substitutions in the antibody binding domain or antigen-binding domains. For example, in one embodiment of the disclosure, there is provided an Vδ1 antibody or antigen-binding fragment thereof, wherein the antibody or antigen-binding fragment thereof comprises the 6 CDR regions of an antibody selected from the group consisting of ADT1-4-2, ADT1-4-105, ADT1-4-107, ADT1-4-110, ADT1-4-112, ADT1-4-117, ADT1-4-19, ADT1-4-21, ADT1-4-31, ADT1-4-139, ADT1-4-4, ADT1-4-143, ADT1-4-53, ADT1-4-173, ADT1-4-8, ADT1-4-82, ADT1-4-83, ADT1-4-3, ADT1-4-84, ADT1-4-86, ADT1-4-95, ADT1-4-1, ADT1-4-6, ADT1-4-138, ADT1-7-10, ADT1-7-15, ADT1-7-17, ADT1-7-18, ADT1-7-19, ADT1-7-20, ADT1-7-22, ADT1-7-23, ADT1-7-42, ADT1-7-3 and ADT1-7-61, wherein the antibody or antigen-binding fragment thereof has up to 10 amino acid substitutions across all of its CDR regions, suitably up to 5 amino acid substitutions or up to 2 amino acid substitutions. In a further embodiment of the disclosure, there is provided an anti-Vδ1 antibody or antigen-binding fragment thereof, wherein the antibody or antigen-binding fragment thereof comprises the VH and VL sequences of an antibody selected from the group consisting of ADT1-4-2, ADT1-4-105, ADT1-4-107, ADT1-4-110, ADT1-4-112, ADT1-4-117, ADT1-4-19, ADT1-4-21, ADT1-4-31, ADT1-4-139, ADT1-4-4, ADT1-4-143, ADT1-4-53, ADT1-4-173, ADT1-4-8, ADT1-4-82, ADT1-4-83, ADT1-4-3, ADT1-4-84, ADT1-4-86, ADT1-4-95, ADT1-4-1, ADT1-4-6, ADT1-4-138, ADT1-7-10, ADT1-7-15, ADT1-7-17, ADT1-7-18, ADT1-7-19, ADT1-7-20, ADT1-7-22, ADT1-7-23, ADT1-7-42, ADT1-7-3 and ADT1-7-61, wherein the antibody has up to 10 amino acid substitutions across its VH and VL sequences, suitably up to 5 amino acid substitutions or up to 2 amino acid substitutions. In a still further embodiment of the disclosure, there is provided an anti-Vδ1 antibody or antigen-binding fragment thereof, wherein the antibody or antigen-binding fragment thereof is an antibody selected from the group consisting of ADT1-4-2, ADT1-4-105, ADT1-4-107, ADT1-4-110, ADT1-4-112, ADT1-4-117, ADT1-4-19, ADT1-4-21, ADT1-4-31, ADT1-4-139, ADT1-4-4, ADT1-4-143, ADT1-4-53, ADT1-4-173, ADT1-4-8, ADT1-4-82, ADT1-4-83, ADT1-4-3, ADT1-4-84, ADT1-4-86, ADT1-4-95, ADT1-4-1, ADT1-4-6, ADT1-4-138, ADT1-7-10, ADT1-7-15, ADT1-7- 17, ADT1-7-18, ADT1-7-19, ADT1-7-20, ADT1-7-22, ADT1-7-23, ADT1-7-42, ADT1-7-3 and ADT1-7-61, wherein the antibody has up to amino acid substitutions, suitably up to 5 amino acid substitutions or up to 2 amino acid substitutions. Substitutions are of course substitutions with reference to the original CDR or variable chain sequences of the starting antibody.

In some embodiments, the one or more amino acid substitutions are in the CDR region or regions. In other embodiments, the one or more amino acid substitutions are in the framework regions, i.e. in the variable heavy and light chains but not in the CDR region or regions. In other embodiments, the one or more amino acid substitutions may be at any position in the variable heavy and/or variable light regions. In some embodiments, the amino acid substitutions do not occur in a CDR sequence.

In some embodiments, the amino acid substitutions do not adversely affect the binding specificity and/or affinity of the antibody. Accordingly, the variant antibody may have the same (or substantially the same) or a superior functional profile as the antibody from which is it derived.

In some embodiments, amino acid substitutions may be made to increase the binding affinity of the antibody to a particular antigen. For example, in embodiments of the disclosure relating to the mutagenesis of the serine at position 74 of the (variable region of the) light chain, a substitution may be made to increase the cross reactivity of the antibody for a the cyno homolog of the human antigen against which the antibody was prepared. Unlike other substitutions described above, this substitution may preferably be non-conservative. In some embodiments, the substitution may be a substitution of the serine at position 74 to a non-polar amino acid (for example to an amino acid selected from the group consisting of glycine, alanine, valine, methionine, leucine and isoleucine). Alternatively, the serine may be substituted with a non-human-germline amino acid (for example to an amino acid selected from the group consisting of arginine, cysteine, glutamine, glutamic acid, glycine, histidine, isoleucine, leucine, lysine, methionine, phenylalanine, threonine, tryptophan, tyrosine and valine). In some embodiments, the serine may be substituted with an amino acid that is both non-human-germline and non-polar, i.e. an amino acid selected from the group consisting of glycine, valine, methionine, leucine and isoleucine. In some embodiments, the serine may be substituted with a leucine.

### Epitopes

Provided herein are multispecific antibodies (or antigen-binding fragments thereof) which bind to an epitope of the Vδ1 chain of a γδ TCR. Such binding may optionally have an effect on γδ TCR activity, such as activation. The multispecific antibodies of the disclosure are specific for the Vδ1 chain of a γδ TCR, and do not bind epitopes of other antigens, such as the Vδ2 chain of a γδ TCR or the Vδ3 chain of a γδ TCR. The antibodies of the present disclosure may be considered agonistic antibodies, at least with respect to the agonistic effect conferred upon Vδ1 cells upon binding.

In one embodiment, the epitope may be an activating epitope of a γδ T cell. An “activating” epitope can include, for example, stimulating a TCR function, such as cell degranulation, TCR downregulation, cytotoxicity, proliferation, mobilisation, increased survival or resistance to exhaustion, intracellular signalling, cytokine or growth factor secretion, phenotypic change, or a change in gene expression. For example, the binding of the activating epitope may stimulate expansion (i.e. proliferation) of the γδ T cell population, preferably the Vδ1+ T cell population. Accordingly, these antibodies can be used to modulate γδ T cell activation, and, thereby, to modulate the immune response. Therefore, in one embodiment, binding of the activating epitope downregulates the γδ TCR. In an additional or alternative embodiment, binding of the activating epitope activates degranulation of the γδ T cell. In a further additional or alternative embodiment, binding of the activating epitope promotes γδ T cell mediated killing. For example, a multispecific antibody that binds to an activating epitope of a γδ T cell, e.g. an activating Vδ1 epitope, is a Vδ1 agonist.

In some embodiments, an activating epitope of TRDV1 is one that, upon being bound by an antibody, results in down-regulation of the receptor and optionally activates the Vδ1 cell. In some embodiments, the activating epitope is one that upregulates expression of activatory markers on the Vδ1 cell, for example CD107a, CD25, CD69 and/or Ki-67. In some embodiments, the activating epitope is one that upregulates expression of activatory markers on the Vδ1 cell, for example CD107 and CD25, and optionally CD69 and/or Ki-67. In some embodiments, upregulation of the one or more activatory markers (such as CD107a) may be upregulation in the presence of cancer cells. In preferred embodiments of the disclosure, the antibodies bind activating epitopes of TRDV1, in particular via the TRDV1-binding domain.

As T-cell receptors are often complexed with other proteins, downregulation of the T-cell receptor via Vδ1 antibody binding may cause downregulation of other proteins associated with the T-cell receptor (i.e. the binding of the Vδ1 antibody causes down regulation of the T-cell receptor complex). For example, in some embodiments, an activating epitope of TRDV1 is one that upon binding, down-regulates the TCR/CD3 receptor complex. In this way, the antibodies of the disclosure may cause indirect downregulation of cell surface proteins that are not bound by the antibody but are complexed to the T-cell receptor. Given T-cells expressing gamma delta 1 chains (i.e. Vδ1 cells) represent only a small number of the total T-cell population, the multispecific antibodies of the disclosure can be used to selectively (and indirectly) downregulate proteins in the TCR complex, such as CD3, by only downregulating them in Vδ1 cells.

In some embodiments, a T-cell receptor complex activating epitope is one that upon activation, downregulates the T-cell receptor complex, whilst not downregulating CD3 molecules not associated with said TRDV1 TCR complex.

The epitope is preferably comprised of or consists of at least one extracellular, soluble, hydrophilic or external portion of the Vδ1 chain of a γδ TCR.

In particular, the epitope does not comprise or consist of an epitope found in a hypervariable region of the Vδ1 chain of the γδ TCR, in particular CDR3 of the Vδ1 chain. In a preferred embodiment, the epitope is within the non-variable region of the Vδ1 chain of the γδ TCR. It will be appreciated that such binding allows for the unique recognition of the Vδ1 chain without the restriction to the sequences of the TCR which are highly variable (in particular CDR3). Various γδ TCR complexes which recognise antigen may be recognised in this way, solely by presence of the Vδ1 chain. As such, it will be appreciated that any Vδ1 chain-comprising γδ TCR may be recognised using the multispecific antibodies as defined herein, irrespective of the specificity of the γδ TCR. In one embodiment, the epitope comprises or consists of one or more amino acid residues within amino acid regions 1-24 and/or 35-90 of SEQ ID NO: 272, e.g. the portions of the Vδ1 chain which are not part of the CDR1 and/or CDR3 sequences. In one embodiment, the epitope does not comprise or consist of amino acid residues within amino acid region 91-105 (CDR3) of SEQ ID NO: 272.

In some embodiments the epitope comprises or consists of amino acids in the TRDV-1 CDR2 sequence.

In a similar manner to the well characterised αβ T cells, γδ T cells utilize a distinct set of somatically rearranged variable (V), diversity (D), joining (J), and constant (C) genes, although γδ T cells contain fewer V, D, and J segments than αβ T cells. In one embodiment, the epitope of Vδ1 bound by the multispecific antibodies (or antigen-binding fragments thereof) does not comprise or consist of an epitope found in the J region of the Vδ1 chain (e.g. one of the four J regions encoded in the human delta one chain germline: SEQ ID NO: 301 (J1*0) or 302 (J2*0) or 303 (J3*0) or 304 (J4*0)) or in the C-region of the Vδ1 chain (e.g. SEQ ID NO: 305 (C1*0) which contains the C-terminal juxtamembrane/transmembrane regions). In one embodiment, the epitope of Vδ1 bound by the multispecific antibodies (or antigen-binding fragments thereof) does not comprise or consist of an epitope found in the N-terminal leader sequence of the Vδ1 chain (e.g. SEQ ID NO: 299). The antibody or fragment may therefore only bind in the V region of the Vδ1 chain (e.g. SEQ ID NO: 300). Thus, in one embodiment, the epitope consists of an epitope in the V region of the γδ TCR (e.g. amino acid residues 1-90 of SEQ ID NO: 272).

Reference to the epitope is made in relation to the Vδ1 sequence derived from the sequence described in Luoma et al. (2013) Immunity 39: 1032-1042, and RCSB Protein Data Bank entries: 3OMZ, shown as SEQ ID NO: 272:

(SEQ ID NO: 272)
AQKVTQAQSSVSMPVRKAVTLNCLYETSWWSYYIFWYKQLPSKEMIFLI
RQGSDEQNAKSGRYSVNFKKAAKSVALTISALQLEDSAKYFCALGESLT
RADKLIFGKGTRVTVEPNIQNPDPAVYQLRDSKSSDKSVCLFTDFDSQT
NVSQSKDSDVYITDKTVLDMRSMDFKSNSAVAWSNKSDFACANAFNNSI
IPEDTFFPSPESS

SEQ ID NO: 272 represents a soluble TCR comprising or consisting of a V region (also referred to as the variable domain), a D region, a J region and a TCR constant region. The V region comprises or consists of amino acid residues 1-90, the D region comprises or consists of amino acid residues 91-104, the J region comprises or consists of amino acid residues 105-115 and the constant region (derived from T-cell receptor alpha) comprises or consists of amino acid residues 116-209. Within the V region, CDR1 is defined as amino acid residues 25-34 of SEQ ID NO: 272, CDR2 is defined as amino acid residues 50-54 of SEQ ID NO: 272, and CDR3 is defined as amino acid residues 93-104 of SEQ ID NO: 272 (Xu et al., PNAS USA 108(6):2414-2419 (2011)).

Therefore, according to an aspect of the disclosure, there is provided an isolated antibody or antigen-binding fragment thereof, which binds to an epitope of a variable delta 1 (Vδ1) chain of a γδ T cell receptor (TCR) comprising or consisting of one or more amino acid residues within amino acid regions:
(i) 3-20 of SEQ ID NO: 272; and/or
(ii) 37-77 of SEQ ID NO: 272.

In a further embodiment, the multispecific antibodies additionally recognize the polymorphic V region comprising or consisting of amino acid residues 1-90 epitope of SEQ ID NO: 306. Hence, amino acids 1-90 of SEQ ID NO: 272 and the polymorphic germline variant sequence (amino acids 1-90 SEQ ID NO: 306) may be considered interchangeable when defining epitopes described herein. Studies presented herein have demonstrated multispecific antibodies of the disclosure can recognize both variants of this germline sequence. By way of example, where it is stated that multispecific antibodies or antigen-binding fragments thereof as defined herein recognize epitopes comprising or consisting of one or more amino acid residues within amino acid regions 1-24 and/or 35-90 of SEQ ID NO: 272, they additionally recognise equivalent epitopes (i.e. at the same position) in SEQ ID NO: 306.

In one embodiment, multispecific antibodies or antigen-binding fragments thereof recognize one or more amino acid residues within amino acid regions 1-90 of SEQ ID NO: 272. More specifically, in one embodiment multispecific antibodies or antigen-binding fragments thereof as defined herein recognize a human germline epitope wherein said germline encodes either an alanine (A) or valine (V) at position 71 of SEQ ID NO: 272.

In one embodiment, the epitope comprises or consists of one or more, such as two, three, four, five, six, seven, eight, nine, ten or more amino acid residues within the described regions.

In a further embodiment, the epitope comprises or consists of one or more (such as 5 or more, such as 10 or more) amino acid residues within amino acid region 3-20 of SEQ ID NO: 272. In an alternative embodiment, the epitope comprises or consists of one or more (such as 5 or more, such as 10 or more) amino acid residues within amino acid region 37-77 of SEQ ID NO: 272 (such as amino acid region 50-54). In a yet further embodiment, the epitope comprises or consists of one or more (such as 5 or more, such as 10 or more) amino acid residues within amino acid region 3-20 (such as 5-20 or 3-17) and one or more (such as 5 or more, such as 10 or more) amino acid residues within amino acid region 37-77 (such as 62-77 or 62-69) of SEQ ID NO: 272.

It will be further understood that said antibody (or antigen-binding fragment thereof) does not need to bind to all amino acids within the defined range. Such epitopes may be referred to as linear epitopes. For example, an antibody which binds to an epitope comprising or consisting of amino acid residues within amino acid region 5-20 of SEQ ID NO: 272 may only bind with one or more of the amino acid residues in said range, e.g. the amino acid residues at each end of the range (i.e. amino acids 5 and 20), optionally including amino acids within the range (i.e. amino acids 5, 9, 16 and 20).

In one embodiment, the epitope comprises or consists of at least one of amino acid residues 3, 5, 9, 10, 12, 16, 17, 20, 37, 42, 50, 53, 59, 62, 64, 68, 69, 72 or 77 of SEQ ID NO: 272. In further embodiments, the epitope comprises or consists of one, two, three, four, five, six, seven, eight, nine, ten, eleven or twelve amino acids selected from amino acid residues 3, 5, 9, 10, 12, 16, 17, 20, 37, 42, 50, 53, 59, 62, 64, 68, 69, 72 or 77 of SEQ ID NO: 272.

In one embodiment, the epitope comprises or consists of one or more amino acid residues within the following amino acid regions of SEQ ID NO: 272:
(i) 3-17;
(ii) 5-20;
(iii) 37-53;
(iv) 50-64;
(v) 59-72;
(vi) 59-77;
(vii) 62-69; and/or
(viii) 62-77.

In further embodiments, the epitope comprises or consists of one or more amino acid residues within amino acid regions: 5-20 and 62-77 (for example, although not limited to, embodiments relating to antibodies derived from the parental clone E07, such as affinity matured variants thereof); 50-64 (for example, although not limited to, embodiments relating to antibodies derived from the parental clone C08, such as affinity matured variants thereof); 37-53 and 59-72 (for example, although not limited to, embodiments relating to antibodies derived from the parental clone G04, such as affinity matured variants thereof); 59-77 (for example, although not limited to, embodiments relating to antibodies derived from the parental clone C05, such as affinity matured variants thereof); or 3-17 and 62-69 (for example, although not limited to, embodiments relating to antibodies derived from the parental clone E01, such as affinity matured variants thereof), of SEQ ID NO: 272. In a further embodiment, the epitope consists of one or more amino acid residues within amino acid regions: 5-20 and 62-77; 50-64; 37-53 and 59-72; 59-77; or 3-17 and 62-69, of SEQ ID NO: 272.

In a further embodiment, the epitope comprises or consists of amino acid residues: 3, 5, 9, 10, 12, 16, 17, 62, 64, 68 and 69 of SEQ ID NO: 272, or suitably consists of amino acid residues: 3, 5, 9, 10, 12, 16, 17, 62, 64, 68 and 69 of SEQ ID NO: 272 (for example, although not limited to, embodiments relating to antibodies derived from the parental clone E01, such as affinity matured variants thereof). In a further embodiment, the epitope comprises or consists of amino acid residues: 5, 9, 16, 20, 62, 64, 72 and 77 of SEQ ID NO: 272, or suitably consists of amino acid residues: 5, 9, 16, 20, 62, 64, 72 and 77 of SEQ ID NO: 272 (for example, although not limited to, embodiments relating to antibodies derived from the parental clone E07, such as affinity matured variants thereof). In yet further embodiment, the epitope comprises or consists of the amino acid residues: 37, 42, 50, 53, 59, 64, 68, 69, 72, 73 and 77 of SEQ ID NO: 272, or suitably consists of amino acid residues: 37, 42, 50, 53, 59, 64, 68, 69, 72, 73 and 77 of SEQ ID NO: 272 (for example, although not limited to, embodiments relating to antibodies derived from the parental clone G04, such as affinity matured variants thereof). In a further embodiment, the epitope comprises or consists of the amino acid residues: 50, 53, 59, 62 and 64 of SEQ ID NO: 272, or suitably consists of amino acid residues: 50, 53, 59, 62 and 64 of SEQ ID NO: 272 (for example, although not limited to, embodiments relating to antibodies derived from the parental clone C08, such as affinity matured variants thereof). In a further embodiment, the epitope comprises or consists of amino acid residues: 59, 60, 68 and 72 of SEQ ID NO: 272, or suitably consists of amino acid residues: 59, 60, 68 and 72 of SEQ ID NO: 272 (for example, although not limited to, embodiments relating to antibodies derived from the parental clone C05, such as affinity matured variants thereof).

In one embodiment, the epitope comprises or consists of one or more amino acid residues within amino acid regions 37-53 and/or 59-77 of SEQ ID NO: 272. In a further embodiment, the epitope consists of one or more amino acid residues within amino acid regions 37-53 and 59-77 of SEQ ID NO: 272. In an alternative further embodiment, the epitope comprises or consists of one or more amino acid residues within amino acid regions 37-53 or 59-77 of SEQ ID NO: 272. Multispecific antibodies or antigen-binding fragments thereof having such epitopes may have some or all of the sequences of G04, or such antibodies or antigen-binding fragments thereof may be derived from G04. For example, multispecific antibodies or antigen-binding fragments thereof having one or more CDR sequences of G04 or one or both of the VH and VL sequences of G04 may bind such epitopes.

In one embodiment, the epitope comprises or consists of one or more amino acid residues within amino acid regions 5-20 and/or 62-77 of SEQ ID NO: 272. In a further embodiment, the epitope consists of one or more amino acid residues within amino acid regions 5-20 and 62-77 of SEQ ID NO: 272. In an alternative further embodiment, the epitope comprises or consists of one or more amino acid residues within amino acid regions 5-20 or 62-77 of SEQ ID NO: 272. Multispecific antibodies or antigen-binding fragments thereof having such epitopes may have some or all of the sequences of E07, or such antibodies or antigen-binding fragments thereof may be derived from E07. For example, multispecific antibodies or antigen-binding fragments thereof having one or more CDR sequences of E07 or one or both of the VH and VL sequences of E07 may bind such epitopes.

In one embodiment, the epitope comprises or consists of one or more amino acid residues within amino acid region 50-64 of SEQ ID NO: 272. In a further embodiment, the epitope consists of one or more amino acid residues within amino acid region 50-64 of SEQ ID NO: 272. Multispecific antibodies or antigen-binding fragments thereof having such epitopes may have some or all of the sequences of C08, or such antibodies or antigen-binding fragments thereof may be derived from C08. For example, multispecific antibodies or antigen-binding fragments thereof having one or more CDR sequences of C08 or one or both of the VH and VL sequences of C08 may bind such epitopes.

In one embodiment, the epitope comprises or consists of one or more amino acid residues within amino acid region 59-72 of SEQ ID NO: 272. In a further embodiment, the epitope consists of one or more amino acid residues within amino acid region 59-72 of SEQ ID NO: 272. Multispecific antibodies or antigen-binding fragments thereof having such epitopes may have some or all of the sequences of C05, or such antibodies or antigen-binding fragments thereof may be derived from C05. For example, multispecific antibodies or antigen-binding fragments thereof having one or more CDR sequences of C05 or one or both of the VH and VL sequences of C05 may bind such epitopes.

In one embodiment, the epitope does not comprise or consist of amino acid residues within amino acid region 11-21 of SEQ ID NO: 272. In one embodiment, the epitope does not comprise or consist of amino acid residues within amino acid region 21-28 of SEQ ID NO: 272. In one embodiment, the epitope does not comprise or consist of amino acid residues within amino acid region 59 and 60 of SEQ ID NO: 272. In one embodiment, the epitope does not comprise or consist of amino acid residues within amino acid region 67-82 of SEQ ID NO: 272.

The epitopes of affinity matured antibodies will generally be the same as the epitopes identified herein for the parental clone. For those epitopes on cyno TRDV1, the positions of the epitopes of the affinity matured antibodies will generally the same positions as the epitopes identified for the corresponding parental clone. The reference to “positions” is necessary since the skilled person would appreciate the identity of some of the amino acids in the epitopes differ from human TRDV1. Despite these variations between human and cyno TRDV1, such antibodies are still able to specifically bind to both antigens.

In one embodiment, the epitope is not the same epitope bound by a commercially available anti-Vδ1 antibody, such as TS-1 or TS8.2. As described in WO2017197347, binding of TS-1 and TS8.2 to soluble TCRs was detected when the 61 chain included Vδ1 J1 and Vδ1 J2 sequences but not to the Vδ1 J3 chain, indicating that the binding of TS-1 and TS8.2 involved critical residues in the delta J1 and delta J2 region.

References to “within” herein include the extremities of the define range. For example, “within amino acid regions 5-20” refers to all of amino acid resides from and including residue 5 up to and including residue 20.

Various techniques are known in the art to establish which epitope is bound by an antibody. Exemplary techniques include, for example, routine cross-blocking assays, alanine scanning mutational analysis, peptide blot analysis, peptide cleavage analysis crystallographic studies and NMR analysis. In addition, methods such as epitope excision, epitope extraction and chemical modification of antigens can be employed. Another method that can be used to identify the amino acids within a polypeptide with which an antibody interacts is hydrogen/deuterium exchange detected by mass spectrometry (as described in Example 9). In general terms, the hydrogen/deuterium exchange method involves deuterium-labelling the protein of interest, followed by binding the antibody to the deuterium-labelled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labelled residues which correspond to the specific amino acids with which the antibody interacts.

The multispecific antibodies and antigen-binding fragments therefor suitably specifically bind to both human TRDV1 (SEQ ID NO: 272 and the polymorphic variant of SEQ ID NO: 306) as well as an ortholog in cynomolgus monkey, cyno TRDV1 (SEQ ID NO: 308 see also UniProtKB—G7P9S6 (G7P9S6_MACFA)). In some embodiments, the multispecific antibodies or antigen-binding fragments thereof are G04-derived antibodies that specifically bind to both human and cyno TRDV1. It is hypothesised the epitopes bound by the multispecific antibodies of the disclosure (for example although not limited to those binding in a region within amino acid resides 37 to 77 of any of SEQ ID NOs: 272, 306 or 308) may be particularly advantageous, since it allows the provision of anti-Vδ1 antibodies that are specific to Vδ1 (i.e. do not bind similar antigens, such as Vδ2 or Vδ3) but also provide cross-reactivity to polymorphic variants of Vδ1 (i.e. TRDV1 SEQ ID NO: 272 and TRDV1 SEQ ID NO: 306, despite the polymorphism at residue position 20 and this being identified as a possible contact residue or close to an identified contact residue for some antibodies) and provides cross-reactivity between human and cyno Vδ1 (despite all of residues 42, 50, 54, 59, 60, 68, 73, 75 and 76 occurring within the region of amino acid resides 37 to 77 of SEQ ID NOs: 272 and 308 being different between human and cyno TRDV1 sequences).

### Epitopes of ADT1-4-Derived Antibodies

The ADT1-4-derived antibodies bind the same or substantially the same epitope as the ADT1-4 parental antibody. Accordingly, in some embodiments, in particular those relating to antibodies derived from or related to the ADT1-4 parental antibody, the anti-Vδ1 antibody or antigen-binding fragment binds an epitope comprising or consisting of one or more amino acid residues within amino acid region 37 to 77, for example within amino acid regions 37-53 and/or 59-77, of SEQ ID NO: 272 (or SEQ ID NO: 306). In some embodiments, the epitope comprises or consists of amino acid residues 37, 42, 50, 53, 59, 64, 68, 69, 72, 73 and 77 of SEQ ID NO: 272 (or SEQ ID NO: 306). In some embodiments, the epitope consists of amino acid residues 37, 42, 50, 53, 59, 64, 68, 69, 72, 73 and 77 of SEQ ID NO: 272 (or SEQ ID NO: 306).

The ADT1-4-derived antibodies also bind to epitope of the cyno variable delta 1 (Vδ1) chain of a γδ T cell receptor (TCR), aka cyno TRDV1 (SEQ ID NO: 308). Hence, in some embodiments, in particular those relating to antibodies derived from or related to the G04 parental antibody, the anti-Vδ1 antibody or antigen-binding fragment binds an epitope comprising or consisting of one or more amino acid residues within amino acid region 37 to 77, for example within amino acid regions 37-53 and/or 59-77, of SEQ ID NO: 308. In some embodiments, the epitope comprises or consists of amino acid residues 37, 42, 50, 53, 59, 64, 68, 69, 72, 73 and 77 of SEQ ID NO: 308. In some embodiments, the epitope consists of amino acid residues 37, 42, 50, 53, 59, 64, 68, 69, 72, 73 and 77 of SEQ ID NO: 308.

Given the cross-reactivity of the ADT1-4-derived antibodies in particular, and the ability of the antibodies disclosed herein to bind to the polymorphic variant of TRDV1, in some embodiments, in particular those relating to antibodies derived from or related to the G04 parental antibody, the anti-Vδ1 antibody or antigen-binding fragment binds an epitope comprising or consisting of one or more amino acid residues within amino acid region 37 to 77, for example within amino acid regions 37-53 and/or 59-77, of SEQ ID NOs: 272, 306 and 308. In some embodiments, the epitope comprises or consists of amino acid residues 37, 42, 50, 53, 59, 64, 68, 69, 72, 73 and 77 of SEQ ID NOs 272, 306 and 308. In some embodiments, the epitope consists of amino acid residues 37, 42, 50, 53, 59, 64, 68, 69, 72, 73 and 77 of SEQ ID NOs 272, 306 and 308.

The provision of antibodies that comprise or consist of such epitopes as those described above yet providing cross-reactivity between human and cyno TRDV1 sequences is surprising, given the location of the amino acid variants between these sequences from the two species.

### Epitopes of ADT1-7-Derived Antibodies

The ADT1-7-derived antibodies bind the same or substantially the same epitope as the ADT1-7 parental antibody. Accordingly, in some embodiments, in particular those relating to antibodies derived from or related to the ADT1-7 parental antibody, the anti-Vδ1 antibody or antigen-binding fragment binds an epitope comprising or consisting of one or more amino acid residues within amino acid regions 5-20 and/or 62-77 of SEQ ID NO: 272 (or SEQ ID NO: 306). In some embodiment, the epitope comprises or consists of amino acid residues 5, 9, 16, 20, 62, 64, 72 and 77 of SEQ ID NO: 272 (or SEQ ID NO: 306). In some embodiments, the epitope consists of amino acid residues 5, 9, 16, 20, 62, 64, 72 and 77 of SEQ ID NO: 272 (or SEQ ID NO: 306).

### Framework and Other Sequences

Suitably the VH and VL regions of the multispecific antibodies or antigen-binding fragments of the disclosure each comprise four framework regions (FR1-FR4). In one embodiment, the antibody or antigen-binding fragment thereof comprises a framework region (e.g. FR1, FR2, FR3 and/or FR4) comprising a sequence having at least 80% sequence identity with the framework region in any one of SEQ ID NOs: 2 to 25 (for example in the case of light chain variable sequences derived from G04), SEQ ID NOs: 27 to 50 (for example in the case of heavy chain variable sequences derived from G04), SEQ ID NOs: 107 to 117 (for example in the case of light chain variable sequence derived from E07) or SEQ ID NOs: 119 to 129 (for example in the case of light chain variable sequence derived from E07). In one embodiment, the antibody or antigen-binding fragment thereof comprises a framework region (e.g. FR1, FR2, FR3 and/or FR4) comprising a sequence having at least 90%, such as at least 95%, 97% or 99% sequence identity with the framework region in any one of SEQ ID NOs: 2 to 25 (for example in the case of light chain variable sequences derived from G04), SEQ ID NOs: 27 to 50 (for example in the case of heavy chain variable sequences derived from G04), SEQ ID NOs: 107 to 117 (for example in the case of light chain variable sequence derived from E07) or SEQ ID NOs: 119 to 129 (for example in the case of light chain variable sequence derived from E07). In one embodiment, the antibody or antigen-binding fragment thereof comprises a framework region (e.g. FR1, FR2, FR3 and/or FR4) comprising a sequence in any one of SEQ ID NOs: 2 to 25 (for example in the case of light chain variable sequences derived from G04), SEQ ID NOs: 27 to 50 (for example in the case of heavy chain variable sequences derived from G04), SEQ ID NOs: 107 to 117 (for example in the case of light chain variable sequence derived from E07) or SEQ ID NOs: 119 to 129 (for example in the case of light chain variable sequence derived from E07). In one embodiment, the antibody or antigen-binding fragment thereof comprises a framework region (e.g. FR1, FR2, FR3 and/or FR4) consisting of a sequence in any one of SEQ ID NOs: 2 to 25 (for example in the case of light chain variable sequences derived from G04), SEQ ID NOs: 27 to 50 (for example in the case of heavy chain variable sequences derived from G04), SEQ ID NOs: 107 to 117 (for example in the case of light chain variable sequence derived from E07) or SEQ ID NOs: 119 to 129 (for example in the case of light chain variable sequence derived from E07).

In some embodiments, the anti-Vδ1 antibody or antigen-binding fragment thereof may comprise an HFR1 (i.e. heavy framework 1 region) sequence comprising or consisting of the sequence of SEQ ID NO: 170 or 171; an HFR2 sequence comprising or consisting of the sequence of SEQ ID NO: 172; an HFR3 sequence comprising or consisting of the sequence of SEQ ID NO: 173; an HFR4 sequence comprising or consisting of the sequence of SEQ ID NO: 174; an LFR1 sequence comprising or consisting of the sequence of SEQ ID NO: 175; an LFR2 sequence comprising or consisting of the sequence of SEQ ID NO: 176; an LFR3 sequence comprising or consisting of the sequence of SEQ ID NO: 177 or 178; and/or an LFR4 sequence comprising or consisting of the sequence of SEQ ID NO: 179, 180, 181 or 182.

In some embodiments, the anti-Vδ1 antibody or antigen-binding fragment thereof may comprise an HFR1 (i.e. heavy framework 1 region) sequence comprising or consisting of the sequence of SEQ ID NO: 170 or 171; an HFR2 sequence comprising or consisting of the sequence of SEQ ID NO: 172; an HFR3 sequence comprising or consisting of the sequence of SEQ ID NO: 173; an HFR4 sequence comprising or consisting of the sequence of SEQ ID NO: 174; an LFR1 sequence comprising or consisting of the sequence of SEQ ID NO: 175; an LFR2 sequence comprising or consisting of the sequence of SEQ ID NO: 176; an LFR3 sequence comprising or consisting of the sequence of SEQ ID NO: 177; and/or an LFR4 sequence comprising or consisting of the sequence of SEQ ID NO: 179, 180, 181 or 182.

In some embodiments, the anti-Vδ1 antibody or antigen-binding fragment thereof may comprise an HFR1 (i.e. heavy framework 1 region) sequence comprising or consisting of the sequence of SEQ ID NO: 170 or 171; an HFR2 sequence comprising or consisting of the sequence of SEQ ID NO: 172; an HFR3 sequence comprising or consisting of the sequence of SEQ ID NO: 173; an HFR4 sequence comprising or consisting of the sequence of SEQ ID NO: 174; an LFR1 sequence comprising or consisting of the sequence of SEQ ID NO: 175; an LFR2 sequence comprising or consisting of the sequence of SEQ ID NO: 176; an LFR3 sequence comprising or consisting of the sequence of SEQ ID NO: 177; and/or an LFR4 sequence comprising or consisting of the sequence of SEQ ID NO: 179 or 181.

In some embodiments, the anti-Vδ1 antibody or antigen-binding fragment thereof may comprise an HFR1 sequence comprising or consisting of the sequence of SEQ ID NO: 189; an HFR2 sequence comprising or consisting of the sequence of SEQ ID NO: 190; an HFR3 sequence comprising or consisting of the sequence of SEQ ID NO: 191; an HFR4 sequence comprising or consisting of the sequence of SEQ ID NO: 192; an LFR1 sequence comprising or consisting of the sequence of SEQ ID NO: 193 and 194; an LFR2 sequence comprising or consisting of the sequence of SEQ ID NO: 195; an LFR3 sequence comprising or consisting of the sequence of SEQ ID NO: 196; and an LFR4 sequence comprising or consisting of the sequence of SEQ ID NO: 197.

In some embodiments, the anti-Vδ1 antibody or antigen-binding fragment thereof may comprise an HFR1 sequence comprising or consisting of the sequence of SEQ ID NO: 189; an HFR2 sequence comprising or consisting of the sequence of SEQ ID NO: 190; an HFR3 sequence comprising or consisting of the sequence of SEQ ID NO: 191; an HFR4 sequence comprising or consisting of the sequence of SEQ ID NO: 192; an LFR1 sequence comprising or consisting of the sequence of SEQ ID NO: 193; an LFR2 sequence comprising or consisting of the sequence of SEQ ID NO: 195; an LFR3 sequence comprising or consisting of the sequence of SEQ ID NO: 196; and an LFR4 sequence comprising or consisting of the sequence of SEQ ID NO: 197.

For fragments comprising both the VH and VL regions, these may be associated either covalently (e.g. via disulphide bonds or a linker) or non-covalently. The antibody fragment described herein may comprise an scFv, i.e. a fragment comprising a VH region and a VL region joined by a linker. In one embodiment, the VH and VL region are joined by a (e.g. synthetic) polypeptide linker. The polypeptide linker may comprise a (Gly₄Ser)ₙ linker (SEQ ID NO: 687), where n=from 1 to 8, e.g. 2, 3, 4, 5 or 7. The polypeptide linker may comprise a [(Gly₄Ser)ₙ(Gly₃AlaSer)ₘ]ₚ linker (SEQ ID NO: 688), where n=from 1 to 8, e.g. 2, 3, 4, 5 or 7, m=from 1 to 8, e.g. 0, 1, 2 or 3, and p=from 1 to 8, e.g. 1, 2 or 3. In a further embodiment, the linker comprises SEQ ID NO: 291. In a further embodiment, the linker consists of SEQ ID NO: 291.

It will be understood by a person skilled in the art that scFv constructs may be designed and made inclusive of N-terminal and C-terminal modifications to aid with translation, purification and detection. For example, at the N-terminus of an scFv sequence, an additional methionine and/or alanine amino acid residue may be included ahead of the canonical VH sequences (e.g. starting QVQ or EVQ). At the C-terminus (i.e. C-terminal to the canonical mature VL domain sequence ending as per the IMGT definition), additional sequences may be included such as (i) a partial sequence of the constant domain and/or (ii) additional synthetic sequences inclusive of tags, such as His-tags and Flag-tags, to aid with purification and detection (for example the tags of any of SEQ ID NOs: 292 to 295).

As described herein, the antibodies may be in any format. In a preferred embodiment, the antibody is in an IgG1 (e.g. human IgG1) format (i.e. the antibody is a human IgG1 antibody).

In some embodiments, the antibody or antigen-binding fragment thereof comprises a light chain constant region comprising the sequence of SEQ ID NO: 296 or SEQ ID NO: 307 (or a sequence having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity with SEQ ID NO: 296 or SEQ ID NO: 307) and/or a heavy chain constant region as defined elsewhere herein (see “Fcabs”). In some embodiments, effector function of the heavy chain constant region may be reduced or disabled (effector function disabling mutations). Suitable mutations to attenuate the effector functions are known to the skilled person. For example, the L235A and/or G237A mutations (“LAGA”) or the L234A and/or L235A mutations (“LALA”) according to the EU numbering.

### Competing Antibodies

In one embodiment, the antibody binds to the same, or essentially the same, epitope as, or competes with, an antibody or antigen-binding fragment thereof as defined herein. One can easily determine whether an antibody binds to the same epitope as, or competes for binding with, a reference anti-Vδ1 antibody by using routine methods known in the art. For example, to determine if a test antibody binds to the same epitope as a reference anti-Vδ1 antibody of the disclosure, the reference antibody is allowed to bind to a Vδ1 protein or peptide under saturating conditions. Next, the ability of a test antibody to bind to the Vδ1 chain is assessed. If the test antibody is able to bind to Vδ1 following saturation binding with the reference anti-Vδ1 antibody, it can be concluded that the test antibody binds to a different epitope than the reference anti-Vδ1 antibody. On the other hand, if the test antibody is not able to bind to the Vδ1 chain following saturation binding with the reference anti-Vδ1 antibody, then the test antibody may bind to the same epitope as the epitope bound by the reference anti-Vδ1 antibody of the disclosure.

The present disclosure also includes anti-Vδ1 antibodies that compete for binding to Vδ1 with an antibody or antigen-binding fragment thereof as defined herein, or an antibody having the CDR sequences of any of the exemplary antibodies described herein. For example, competitive assays can be performed with the antibody of the present disclosure in order to determine what proteins, antibodies, and other antagonists compete for binding to the Vδ1 chain with the antibody of the present disclosure and/or share the epitope. These assays are readily known to those of skill in the art; they evaluate competition between antagonists or ligands for a limited number of binding sites on a protein, e.g. Vδ1. The antibody (or antigen-binding fragment thereof) is immobilized or insolubilized before or after the competition and the sample bound to the Vδ1 chain is separated from the unbound sample, for example, by decanting (where the antibody was pre-insolubilized) or by centrifuging (where the antibody was precipitated after the competitive reaction). Also, the competitive binding may be determined by whether the function is altered by the binding or lack of binding of the antibody to the protein, e.g. whether the antibody molecule inhibits or potentiates the enzymatic activity of, for example, a label. ELISA and other functional assays may be used, as known in the art and described herein.

Two antibodies bind to the same or overlapping epitope if each competitively inhibits (blocks) binding of the other to the target antigen. That is, a 1-, 5-, 10-, 20- or 100-fold excess of one antibody inhibits binding of the other by at least 50% but preferably 75%, 90% or even 99% as measured in a competitive binding assay. Alternatively, two antibodies have the same epitope if essentially all amino acid mutations in the target antigen that reduce or eliminate binding of one antibody reduce or eliminate binding of the other.

Additional routine experimentation (e.g. peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Antibody Binding and Pharmacological Properties of the Antibodies

The multispecific antibodies of the disclosure may have favourable binding and/or pharmacological properties, for example as described below.

In some embodiments, the binding affinity of the antibody or antigen-binding fragment thereof is established by coating the antibody or antigen-binding fragment thereof directly or indirectly (e.g. by capture with an anti-human IgG Fc) onto the surface of a sensor (e.g. an amine high capacity chip or equivalent), wherein the target bound by the antibody or antigen-binding fragment thereof (i.e. the Vδ1 chain of a γδ TCR or PD-L1) is flowed over the chip to detect binding. Suitably, a MASS-2 instrument (which may also be referred to as Sierra SPR-32) is used at 25° C. in PBS+0.02% Tween 20 (Polysorbate 20) running buffer at 30 μl/min.

Described herein (also see Examples section) are other assays which may be used to define antibody function. For example, the multispecific antibody or antigen-binding fragment thereof described herein may be assessed by γδ TCR engagement, e.g. measuring downregulation of the γδ TCR upon antibody binding. Surface expression of the γδ TCR following application of the antibody or antigen-binding fragment thereof (optionally presented on the surface of a cell) can be measured, e.g. by flow cytometry. The antibody or antigen-binding fragment thereof described herein may also be assessed by measuring γδ T cell degranulation. For example, expression of CD107a, a marker for cell degranulation, can be measured following application of the antibody or antigen-binding fragment thereof (optionally presented on the surface of a cell) to γδ T cells, e.g. by flow cytometry. The antibody or antigen-binding fragment thereof described herein may also be assessed by measuring γδ T cell mediated killing activity (to test if the antibody has an effect on the killing activity of the γδ T cell). For example, target cells may be incubated with γδ T cells in the presence of the antibody or antigen-binding fragment thereof (optionally presented on the surface of a cell). Following incubation, the culture may be stained with a cell viability dye to distinguish between live and dead target cells. The proportion of dead cells can then be measured, e.g. by flow cytometry.

Additionally, described herein are assays which may be used to define multispecific antibody function as assessed in assays comprising a mixture of at least two different cell types comprising separate γδ TCR+ve cells and PD-L1+ve cells. These assays help to further highlight some of the surprising technical effects of the multispecific antibodies described herein, whereby further enhanced effects are observed (e.g. increased TCR downregulation) when multispecific antibody function is assessed and defined in assays comprising such cell mixtures.

### Binding Affinity (K_{D}) for Human TRDV1

Generally speaking, the affinity matured clones (and multispecific antibodies derived or related to such clones) will have an affinity for their antigen(s) that is higher than the parental clones. For example, the affinity for human TRDV1 (SEQ ID NOs 272 or 306) will be at least 20%, at least 30%, at least 40%, at least 50%, at least 100% or at least 500% greater affinity than the parental antibody.

The multispecific antibodies or antigen-binding fragments thereof of the disclosure may bind to human TRDV1 (SEQ ID NOs 272 or 306) with a binding affinity (KD, for example as measured by surface plasmon resonance) of less than about 100 nM, preferably less than about 50 nM.

The multispecific antibodies or antigen-binding fragments thereof may further be defined as binding to human TRDV1 (SEQ ID NOs 272 or 306) with a binding affinity (KD, for example as measured by surface plasmon resonance) of less than about 10 nM, preferably less than about 5 nM.

In some embodiments, for example multispecific antibodies or antigen-binding fragments thereof relating to or derived from the ADT1-4 lineage (e.g. multispecific antibodies of the disclosure with a Fab region relating to or derived from the ADT1-4 lineage, for example as a result of affinity maturation or otherwise), the multispecific antibodies or antigen-binding fragments thereof of the disclosure may bind to human TRDV1 with KD of less than about 100 nM, preferably less than about 50 nM.

In some embodiments, for example multispecific antibodies or antigen-binding fragments thereof relating to or derived from the ADT1-4 lineage (but excluding ADT1-4-138), the multispecific antibodies or antigen-binding fragments thereof of the disclosure may bind to human TRDV1 with KD of less than about 10 nM, preferably less than about 5 nM.

In some embodiments, for example multispecific antibodies or antigen-binding fragments thereof relating to or derived from the ADT1-4 lineage members ADT1-4-19, ADT1-4-21, ADT1-4-31, ADT1-4-53, ADT1-4-2, ADT1-4-86, ADT1-4-112, ADT1-4-143 and/or ADT1-4-1, the multispecific antibodies or antigen-binding fragments thereof of the disclosure may bind to human TRDV1 with KD of less than about 10 nM, preferably less than about 1 nM.

The multispecific antibodies or antigen-binding fragments thereof of the disclosure may bind to cyno TRDV1 (SEQ ID NO 308) with a binding affinity (K_{D}, for example as measured by surface plasmon resonance) of less than about 100 nM, preferably less than about 50 nM.

In some embodiments, for example multispecific antibodies or antigen-binding fragments thereof relating to or derived from the ADT1-4 lineage, the multispecific antibodies or antigen-binding fragments thereof of the disclosure may bind to cyno TRDV1 with K_{D} of less than about 100 nM, preferably less than about 50 nM.

In some embodiments, for example multispecific antibodies or antigen-binding fragments thereof relating to or derived from the ADT1-4 lineage members ADT1-4-19, ADT1-4-21, ADT1-4-31, ADT1-4-53, ADT1-4-2, ADT1-4-86, ADT1-4-112, ADT1-4-143 and/or ADT1-4-1, the multispecific antibodies or antigen-binding fragments thereof of the disclosure may bind to cyno TRDV1 with K_{D} of less than about 50 nM.

In some embodiments, for example those relating to or derived from the ADT1-4 lineage, the multispecific antibodies or antigen-binding fragments thereof of the disclosure may bind to human TRDV1 with K_{D} of less than about 100 nM, preferably less than about 50 nM and bind to cyno TRDV1 with a binding affinity (K_{D}, for example as measured by surface plasmon resonance) of less than about 100 nM, preferably less than about 50 nM.

In some embodiments, for example multispecific antibodies or antigen-binding fragments thereof relating to or derived from the ADT1-4 lineage (but excluding ADT1-4-138), the multispecific antibodies or antigen-binding fragments thereof of the disclosure may bind to human TRDV1 with K_{D} of less than about 10 nM and bind to cyno TRDV1 with a binding affinity (K_{D}, for example as measured by surface plasmon resonance) of less than about 100 nM. Preferably in this embodiment, the multispecific antibodies or antigen-binding fragments thereof of the disclosure may bind to human TRDV1 with K_{D} of less than about 5 nM and bind to cyno TRDV1 with a binding affinity (K_{D}, for example as measured by surface plasmon resonance) of less than about 50 nM.

In some embodiments, for example multispecific antibodies or antigen-binding fragments thereof relating to or derived from the ADT1-4 lineage members ADT1-4-19, ADT1-4-21, ADT1-4-31, ADT1-4-53, ADT1-4-2, ADT1-4-86, ADT1-4-112, ADT1-4-143 and/or ADT1-4-1, the multispecific antibodies or antigen-binding fragments thereof of the disclosure may bind to human TRDV1 with K_{D} of less than about 10 nM and bind to human TRDV1 with K_{D} of less than 50 nM. Preferably in this embodiment, the multispecific antibodies or antigen-binding fragments thereof of the disclosure may bind to human TRDV1 with K_{D} of less than about 1 nM and bind to cyno TRDV1 with a binding affinity (K_{D}, for example as measured by surface plasmon resonance) of less than about 50 nM.

In some embodiments, for example multispecific antibodies or antigen-binding fragments thereof relating to or derived from the ADT1-7 lineage (e.g. multispecific antibodies of the disclosure with a Fab region relating to or derived from the ADT1-7 lineage, for example as a result of affinity maturation or otherwise), the multispecific antibodies or antigen-binding fragments thereof of the disclosure may bind to human TRDV1 with K_{D} of less than about 10 nM, preferably less than about 5 nM.

### Binding Affinity (K_{D}) for PD-L1

In some embodiments, the multispecific antibodies of the disclosure may bind to PD-L1 with a K_{D} of less than about 150 nM, or a K_{D} of less than about 20 nM, or a K_{D} of less than about 10 nM, or a K_{D} of less than about 5 nM, or a K_{D} of less than about 4 nM for example ADT1-4-2/ADT5-1 n, ADT1-4-2/ADT5-2n, ADT1-4-2/ADT5-3n, ADT1-4-2/ADT5-4n, ADT1-4-2/ADT5-5n, ADT1-4-2/ADT5-6n, ADT1-4-2/ADT5-7n, ADT1- 4-2/ADT5-8n, ADT1-4-2/ADT5-9n, ADT1-4-2/ADT5-10n, ADT1-4-1/ADT5-1 n, ADT1-4-1/ADT5-2n, ADT1-4-1/ADT5-3n, ADT1-4-1/ADT5-4n, ADT1-4-1/ADT5-5n, ADT1-4-1/ADT5-6n, ADT1-4-1/ADT5-7n, ADT1- 4-1/ADT5-8n, ADT1-4-1/ADT5-9n, ADT1-4-1/ADT5-1 On.

In any embodiment, the PD-L1 may be human PD-L1.

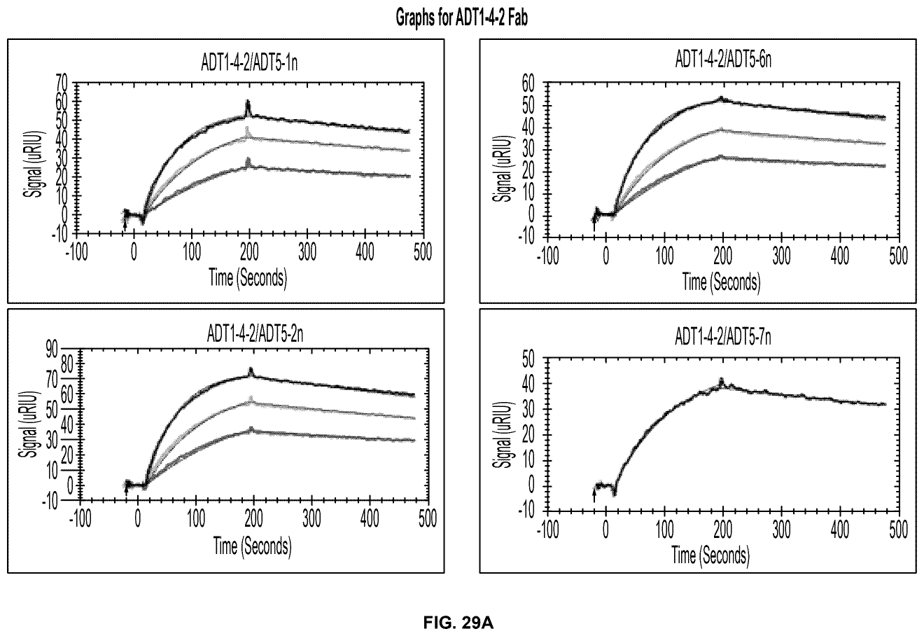

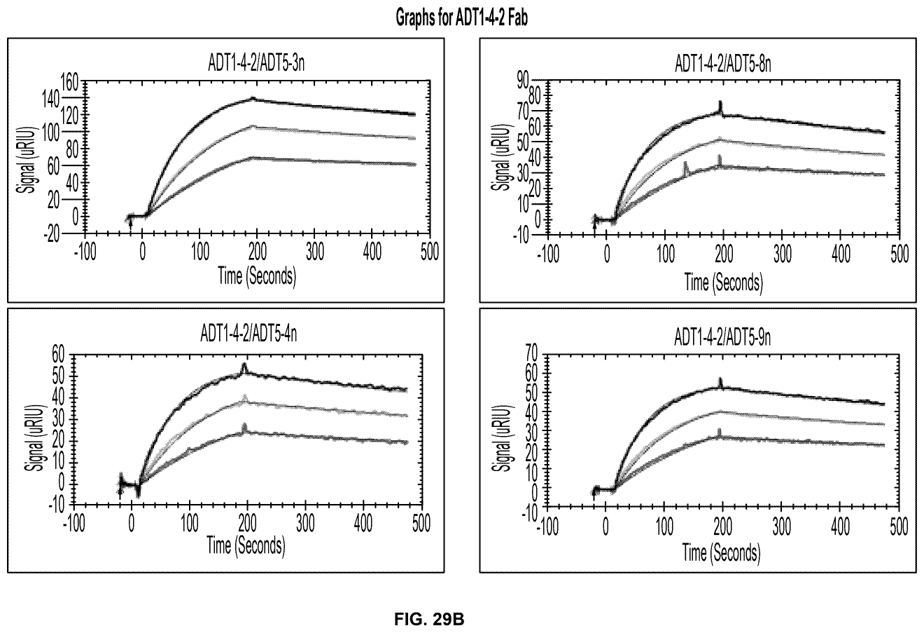

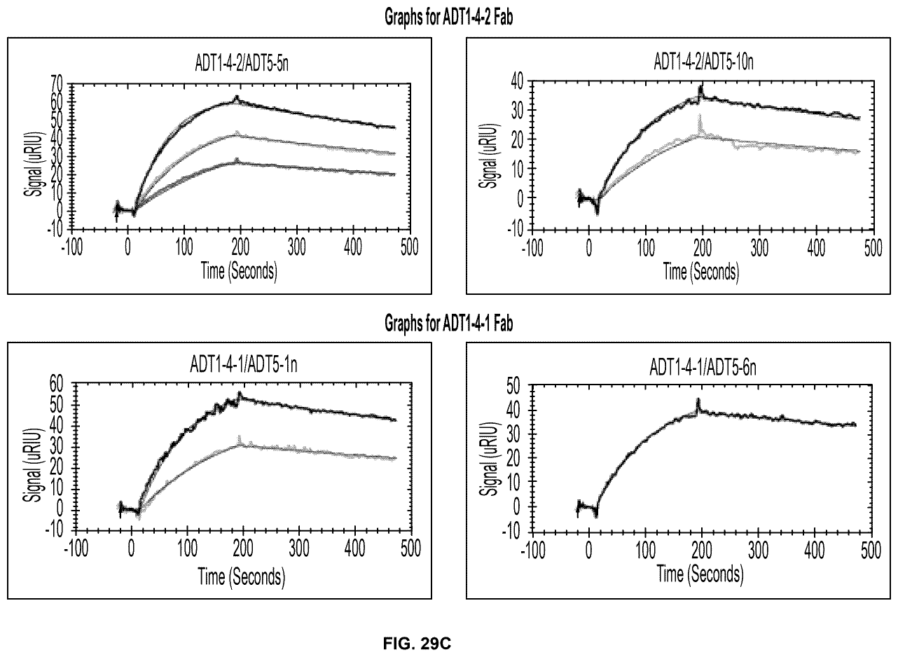

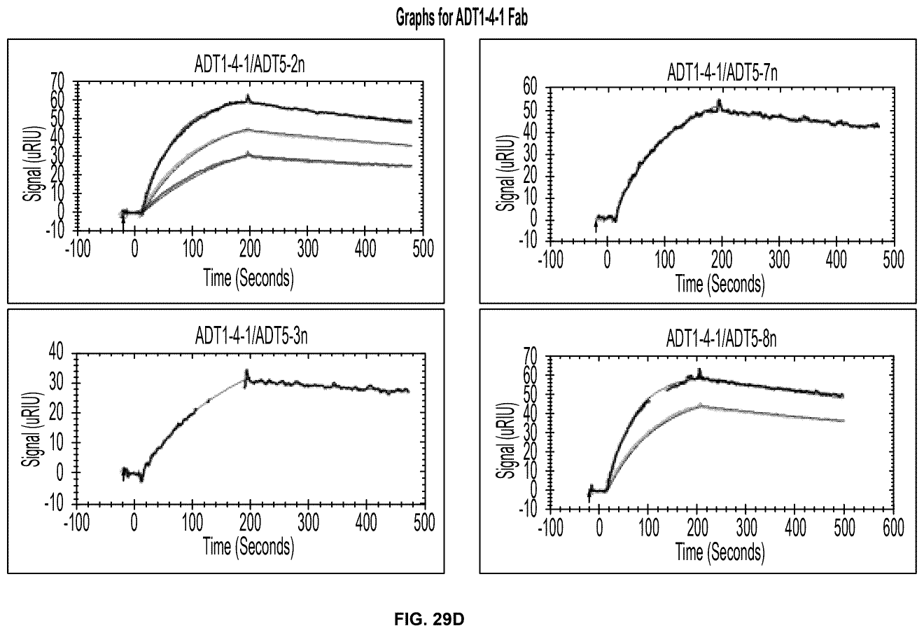

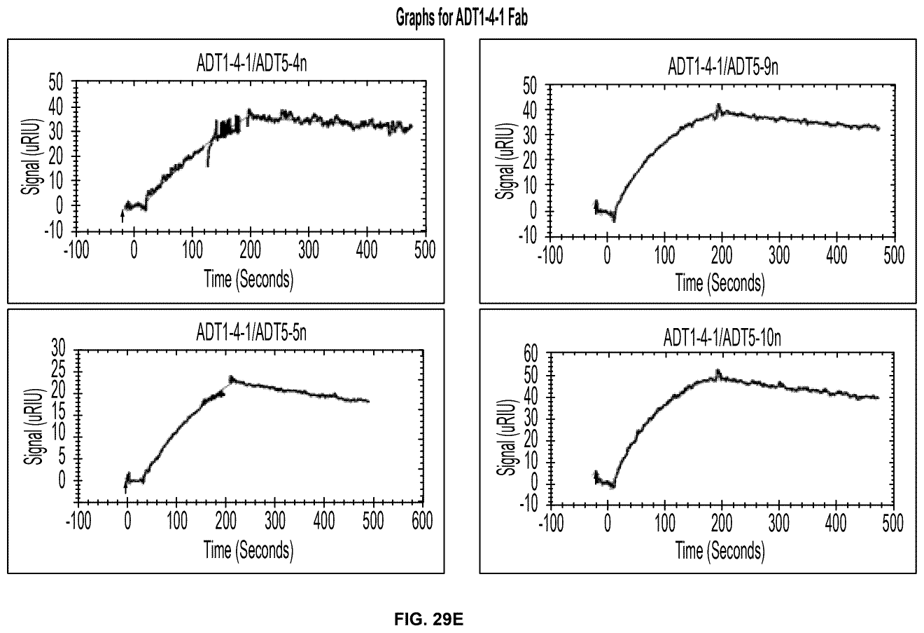

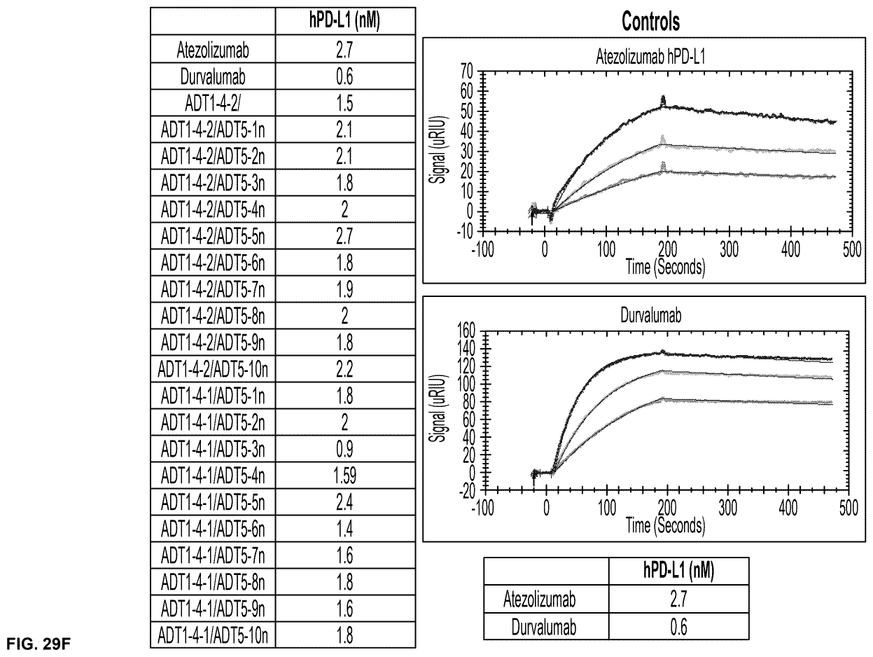

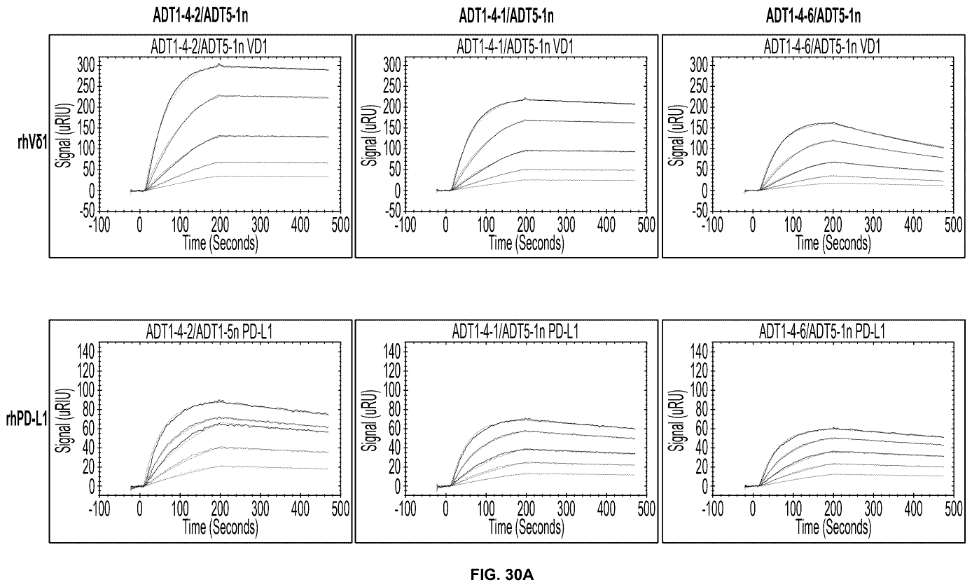

FIGS. 29A-29F provide a summary of the PD-L1 affinities of exemplar multispecific antibodies of the disclosure, as determined by SPR.

In some embodiments, the multispecific antibodies of the disclosure may bind to human TRDV1 with a K_{D} of less than about 5 nM and may bind to PD-L1 with a K_{D} of less than about 150 nM, or a K_{D} of less than about 20 nM, or a K_{D} of less than about 10 nM, or a K_{D} of less than about 5 nM, or a K_{D} of less than about 4 nM for example ADT1-4-2/ADT5-1n, ADT1-4-2/ADT5-2n, ADT1-4-2/ADT5-3n, ADT1-4-2/ADT5-4n, ADT1-4-2/ADT5-5n, ADT1-4-2/ADT5-6n, ADT1-4-2/ADT5-7n, ADT1-4-2/ADT5-8n, ADT1-4-2/ADT5-9n, ADT1-4-2/ADT5-10n, ADT1-4-1/ADT5-1 n, ADT1-4-1/ADT5-2n, ADT1-4-1/ADT5-3n, ADT1-4-1/ADT5-4n, ADT1-4-1/ADT5-5n, ADT1-4-1/ADT5-6n, ADT1-4-1/ADT5-7n, ADT1-4-1/ADT5-8n, ADT1-4-1/ADT5-9n, ADT1-4-1/ADT5-1 On.

### Dialing Up and Down Affinity of Binding Domains

Multispecific antibodies as presented herein comprise at least one first binding domain which binds the TRDV1 domain of Vδ1 TCR and at least one second binding domain which binds PD-L1. Each binding domain binds to its target with a different binding affinity (Kd). These binding affinities can be altered independently of each other. Fine-tuning affinities up or down to both the first binding domain and to the second binding domain impact functionality and conferred technical effects.

TABLE 6
Summary of the human TRDV1 and PD-L1 affinities
of exemplar multispecific antibodies of
the disclosure, as determined by SPR.

| | Vδ1 KD | PD-L1 KD | |
|---|---|---|---|
| Antibody | [nM] | [nM] | Notes |
| ADT1-4-2/ADT5-1n | 0.75 | 2.7 | ‘High Vδ1 affinity’ |
| ADT1-4-1/ADT5-1n | 0.97 | 2 | ‘High Vδ1 affinity’ |
| ADT1-4-6/ADT5-1n | 7.4 | 2.7 | ‘Mid Vδ1 affinity’ |
| ADT1-4-138/ADT5-1n | 60 | 1.8 | ‘Low Vδ1 affinity’ |
| RSV/ADT5-1n | n/a | 2.1 | |

Surprisingly, even when the affinity of the first binding domain (to the TRDV1 domain of Vδ1 TCR) was dialed up and down, the affinity of the second binding domain (PD-L1) was largely unaffected.

### Inhibition Concentrations (IC50)

### TCR Downregulation

TCR down regulation may be measured according to the assays described herein. For example, the antibody to be tested may be incubated at different concentration with a culture of γδ T cells and the downregulation measured. If measuring cell killing (for example THP-1 cell killing, or A375 cell killing), the γδ T cells are co-cultured with a suitable cell line, for example THP-1 cells or A375 cells. TCR down regulation may be measured flow cytometry. Cell killing may be achieved by any suitable means, for example by flow cytometry.

For the initial TCR downregulation studies with the parental antibodies (e.g. ADT1-7 and ADT1-4), the TCR downregulation assay involved ‘loading’ the antibody onto Fc gamma receptor +ve THP-1 cells (see Example 1, Example 6 and Table 5 of WO2021/032963). As such, the antibodies in this instance are presented on a cell surface ahead of co-incubation with γδ T cells. Such loading thereby affords maximum opportunity to exploit cross-linking effects upon TCR engagement. Aside loading antibodies onto Fc receptor +ve cells, alternate similar approaches to presenting the antibody on sold-surfaces may include pre-incubating the antibody on a plate (so-called plate-bound), or the use of carrier beads to present the antibody. In all such assays, and once presented on a solid surface, it is then typical to investigate and measure the technical effect conferred by the antibody upon engagement of the target receptor (in this instance a γδ T cell receptor), Presenting antibodies in this way is commonplace, particularly when exploring the effects of antibody engagement of immune cell receptor targets and complexes such as antibody engagement of targets such as CD3, CD28 etc.

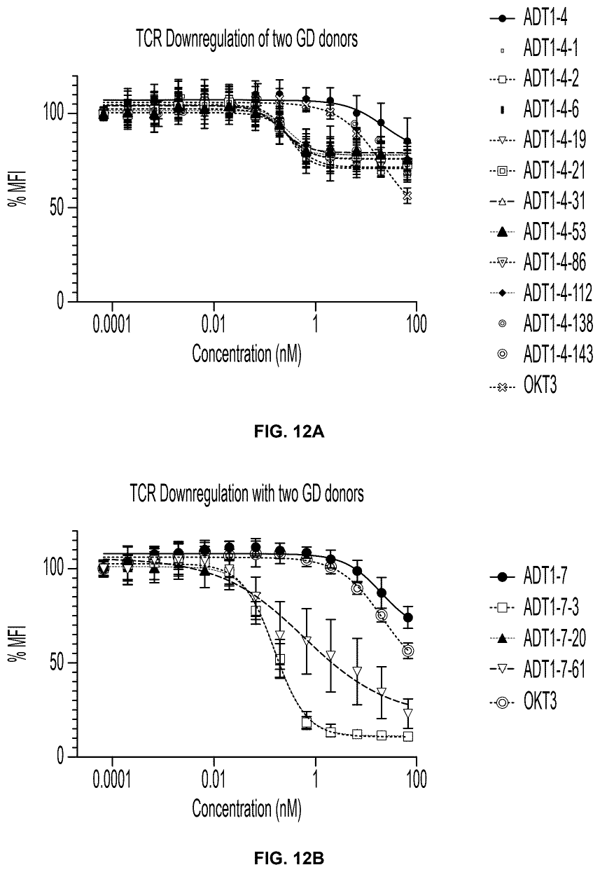

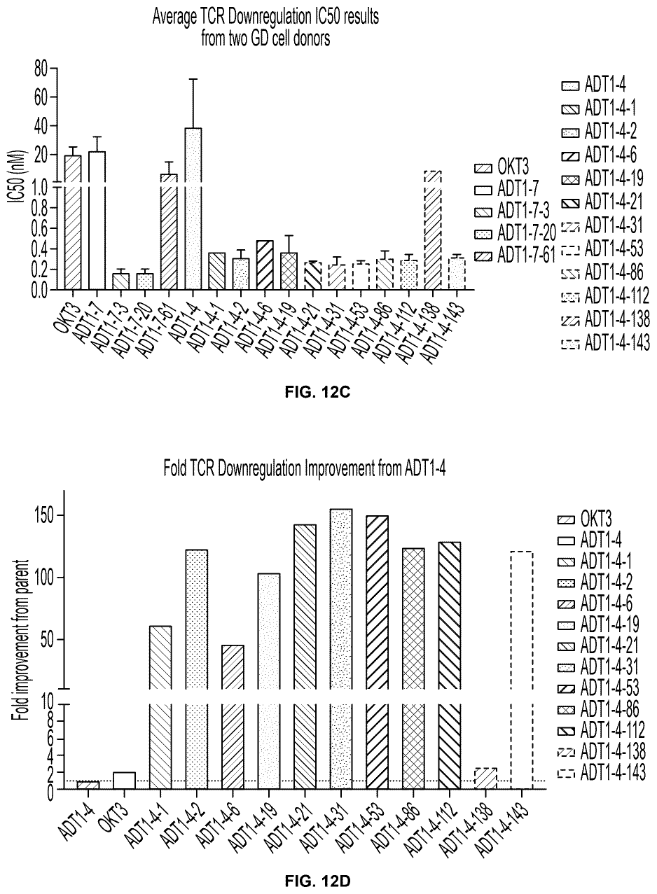

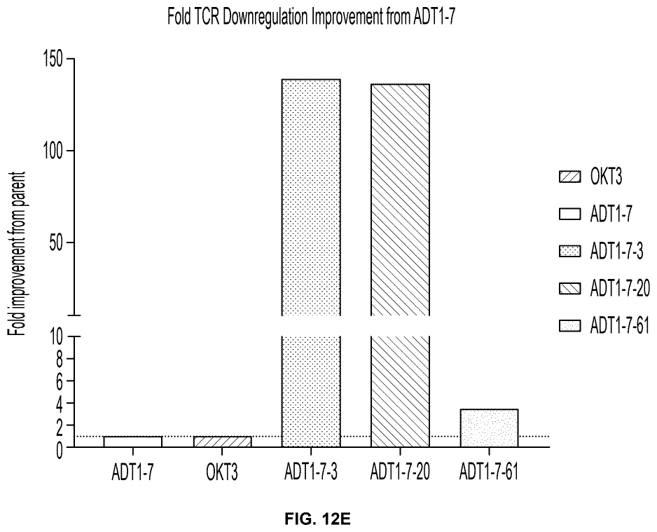

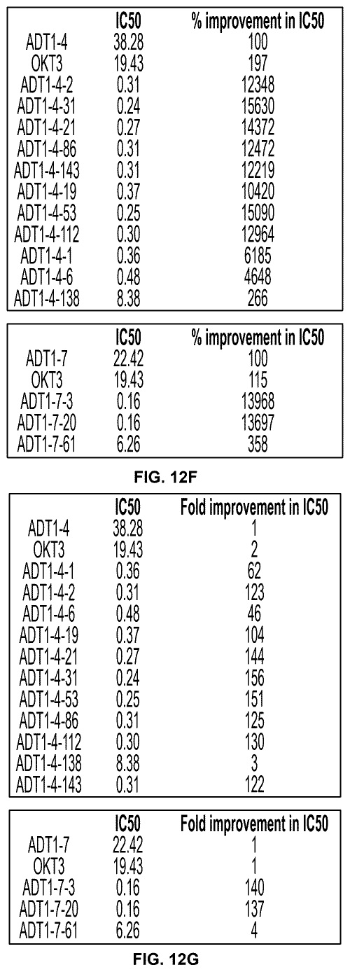

In contrast, for affinity matured antibodies of the disclosure as described herein, we wanted to dissect and compare the impact of affinity maturation to its fullest and hence we explored the capabilities of these antibodies in more ‘soluble’ TCR downregulation assay formats. Assessing the effect in solution may also be more physiologically relevant. For these reasons, and unless otherwise indicated, in all TCR downregulation cell-based experiments wherein the effects of affinity-matured antibodies, multispecific antibodies, or fragments thereof, are measured or characterized and compared to parent antibodies, a soluble assay format is explored. The IC50 difference of employing this more stringent soluble assay is summarized below wherein the TCR downregulation of EC50 values for an example parent molecule (ADT1-4) is summarised via both approaches.
TCR Downregulation: ‘Presented’ versus ‘Soluble’ Assay Format:
ADT1-4 IC50 (loaded on THP-1 cells): 0.01-0.05 ug/ml=0.065 nM to 0.325 nM (See Example 1, Example 6, and Table 5 of WO2021/032963)
Vs.
ADT1-4 IC50 (soluble assay, added in solution)=38.28 nM
(See FIGS. 12A-12G)

The multispecific antibodies or antigen-binding fragments thereof of the disclosure may have an IC50 for TCR downregulation of less than about 50 nM, less than about 10 nM, or less than about 1 mM. Preferably the IC50 is less than about 1 nM.

In some embodiments, for example multispecific antibodies or antigen-binding fragments thereof relating to or derived from the ADT1-4 lineage (e.g. multispecific antibodies of the disclosure with a Fab region relating to or derived from the ADT1-4 lineage, for example as a result of affinity maturation or otherwise), the multispecific antibodies or antigen-binding fragments thereof of the disclosure may have an IC50 for TCR downregulation of less than about 50 nM, less than about 10 nM, or less than about 1 mM. Preferably the IC50 is less than about 1 nM.

In some embodiments, for example multispecific antibodies or antigen-binding fragments thereof relating to or derived from the ADT1-4 lineage (but excluding ADT1-4-138), the multispecific antibodies or antigen-binding fragments thereof of the disclosure may have an IC50 for TCR downregulation of less than about 1 nM. Preferably the IC50 is less than about 0.5 nM.

In some embodiments, for example multispecific antibodies or antigen-binding fragments thereof relating to or derived from the ADT1-4 lineage members ADT1-4-19, ADT1-4-21, ADT1-4-31, ADT1-4-53, ADT1-4-2, ADT1-4-86, ADT1-4-112, ADT1-4-143 and/or ADT1-4-1, the antibodies or multispecific antigen-binding fragments thereof of the disclosure may have an IC50 for TCR downregulation of less than about 1 nM. Preferably the IC50 is less than about 0.5 nM.

In some embodiments, for example multispecific antibodies or antigen-binding fragments thereof relating to or derived from the ADT1-7 lineage (e.g. multispecific antibodies of the disclosure with a Fab region relating to or derived from the ADT1-7 lineage, for example as a result of affinity maturation or otherwise), the multispecific antibodies or antigen-binding fragments thereof of the disclosure may have an IC50 for TCR downregulation of less than about 50 nM, less than about 10 nM, or less than about 1 mM. Preferably the IC50 is less than about 1 nM.

In some embodiments, for example multispecific antibodies or antigen-binding fragments thereof relating to or derived from the ADT1-7 lineage members ADT1-7-20 or ADT1-7-3, the multispecific antibodies or antigen-binding fragments thereof of the disclosure may have an IC50 for TCR downregulation of less than about 5 nM. Preferably the IC50 is less than about 10 nM.

### Cell Killing

Cell killing may be measured according to the assays described herein. For example, the antibody to be tested may be incubated at different concentration with a co-culture of γδ T cells and tumour cells (for example THP-1 cells or A375 cells). Cell killing may be measured by any suitable means, for example by flow cytometry.

The multispecific antibodies or antigen-binding fragments thereof of the disclosure may have an IC50 for A375 cell killing of less than about 10 nM, less than about 5 nM, less than about 1 nM, less than about 0.1 nM or less than about 0.01 nM. Preferably the IC50 is less than about 1 nM.

In some embodiments, for example multispecific antibodies or antigen-binding fragments thereof relating to or derived from the ADT1-4 lineage (e.g. multispecific antibodies of the disclosure with a Fab region relating to or derived from the ADT1-4 lineage, for example as a result of affinity maturation or otherwise), the multispecific antibodies or antigen-binding fragments thereof of the disclosure may have an IC50 for A375 cell killing of less than about 10 nM, less than about 5 nM, less than about 1 nM, less than about 1 nM, less than about 0.1 nM or less than about 0.01 nM. Preferably the IC50 is less than about 1 nM.

In some embodiments, for example multispecific antibodies or antigen-binding fragments thereof relating to or derived from the ADT1-7 lineage (e.g. multispecific antibodies of the disclosure with a Fab region relating to or derived from the ADT1-7 lineage, for example as a result of affinity maturation or otherwise), the multispecific antibodies or antigen-binding fragments thereof of the disclosure may have an IC50 for A375 cell killing of less than about 5 nM, less than about 1 nM, or less than about 0.1 nM. Preferably the IC50 is less than about 1 nM.

In some embodiments, for example multispecific antibodies or antigen-binding fragments thereof relating to or derived from the ADT1-7 lineage members ADT1-7-20 or ADT1-7-3, the multispecific antibodies or antigen-binding fragments thereof of the disclosure may have an IC50 for A375 cell killing of less than about 10 nM, less than about 5 nM, less than about 1 nM or less than about 0.1 nM. Preferably the IC50 is less than about 1 nM.

Of course, the advantageous pharmacological profiles of the multispecific antibodies can be combined such that the antibodies exhibit an advantageous K_{D} and advantageous IC50 values for the various tested properties.

For example, in some embodiments, the multispecific antibodies or antigen-binding fragments thereof of the disclosure may:
bind to human TRDV1 (SEQ ID NOs 272 or 306) with a binding affinity (K_{D}, for example as measured by surface plasmon resonance) of less than about 100 nM (preferably less than about 50 nM);
optionally bind to cyno TRDV1 (SEQ ID NO 308) with a binding affinity (K_{D}, for example as measured by surface plasmon resonance) of less than about 100 nM (preferably less than about 50 nM);
have an IC50 for TCR downregulation of less than about 50 nM (preferably less than about 1 nM);
have an IC50 for A375 cell killing of less than about 10 nM (preferably less than about 5 nM).

In some embodiments, in particular those relating to the ADT1-4 lineage, the multispecific antibodies or antigen-binding fragments thereof may:
bind to human TRDV1 with a K_{D} of less than about 100 nM (preferably less than about 50 nM);
bind to cyno TRDV1 with a K_{D} of less than about 100 nM (preferably less than about 50 nM);
have an IC50 for TCR downregulation of less than about 50 nM (preferably less than about 1 nM);
have an IC50 for A375 cell killing of less than about 10 nM (preferably less than about 5 nM).

In some embodiments, for example multispecific antibodies or antigen-binding fragments thereof relating to or derived from the ADT1-4 lineage (but excluding ADT1-4-138), the multispecific antibodies or antigen-binding fragments thereof of the disclosure may:
bind to human TRDV1 with a K_{D} of less than about 10 nM (preferably less than about 5 nM);
bind to cyno TRDV1 with a K_{D} of less than about 100 nM (preferably less than about 50 nM);
have an IC50 for TCR downregulation of less than about 1 nM (preferably less than about 0.5 nM)
have an IC50 for A375 cell killing of less than about 10 nM (preferably less than about 5 nM).

In some embodiments, for example multispecific antibodies or antigen-binding fragments thereof relating to or derived from the ADT1-4 lineage members ADT1-4-19, ADT1-4-21, ADT1-4-31, ADT1-4-53, ADT1-4-2, ADT1-4-86, ADT1-4-112, ADT1-4-143 and/or ADT1-4-1, the multispecific antibodies or antigen-binding fragments thereof of the disclosure may:
bind to human TRDV1 with a K_{D} of less than about 10 nM (preferably less than about 1 nM);
bind to cyno TRDV1 with a K_{D} of less than about 50 nM;
have an IC50 for TCR downregulation of less than about 1 nM (preferably less than about 0.5 nM)
have an IC50 for A375 cell killing of less than about 10 nM (preferably less than about 5 nM).

In some embodiments, in particular those relating to the ADT1-7 lineage, the multispecific antibodies or antigen-binding fragments thereof may:
bind to human TRDV1 with a K_{D} of less than about 10 nM (preferably less than about 5 nM);
have an IC50 for TCR downregulation of less than about 50 nM (preferably less than about 10 nM);
have an IC50 for A375 cell killing of less than about 5 nM (preferably less than about 1 nM).

In some embodiments, in particular those relating to the ADT1-7 lineages ADT1-7-20 or ADT1-7-3, the multispecific antibodies or antigen-binding fragments thereof may:
bind to human TRDV1 with a K_{D} of less than about 10 nM (preferably less than about 5 nM);
have an IC50 for TCR downregulation of less than about 5 nM (preferably less than about 1 nM);
have an IC50 for A375 cell killing of less than about 10 nM (preferably less than about 5 nM).
   Pharmacological Properties of Antibodies Comprising Fab Regions of or Derived from the ADT1-4 Lineage

The pharmacological properties of the antibodies with respect to their binding (via the Fab region) to TRDV1 may fall within certain parameters. For example, antibodies of or derived from ADT1-4-105 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-107 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-110 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-112 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM. Such antibodies may alternatively or additionally have an IC50 for TCR down regulation of less than about 1 nM (preferably less than about 0.5 nM).

Antibodies of or derived from ADT1-4-117 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-19 (for example fragments thereof, variants having one or more amino acid substitutions or having certain percent identity thereto) may have a K_{D} for human TRDV1 of less than about 1 nM (preferably less than about 0.5 nM) and/or a K_{D} for cyno TRDV1 of less than about 10 nM (preferably less than about 5 nM). Such antibodies may alternatively or additionally have an IC50 for TCR down regulation of less than about 1 nM (preferably less than about 0.5 nM).

Antibodies of or derived from ADT1-4-21 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM (preferably less than about 10 nM). Such antibodies may alternatively or additionally have an IC50 for TCR down regulation of less than about 1 nM (preferably less than about 0.5 nM).

Antibodies of or derived from ADT1-4-31 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM (preferably less than about 10 nM). Such antibodies may alternatively or additionally have an IC50 for TCR down regulation of less than about 1 nM (preferably less than about 0.5 nM).

Antibodies of or derived from ADT1-4-139 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-4 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-143 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM (preferably less than about 10 nM). Such antibodies may alternatively or additionally have an IC50 for TCR down regulation of less than about 1 nM (preferably less than about 0.5 nM).

Antibodies of or derived from ADT1-4-53 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM (preferably less than about 10 nM). Such antibodies may alternatively or additionally have an IC50 for TCR down regulation of less than about 1 nM (preferably less than about 0.5 nM).

Antibodies of or derived from ADT1-4-173 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-2 (for example fragments thereof, variants having one or more amino acid substitutions or having certain percent identity thereto) may have a K_{D} for human TRDV1 of less than about 1 nM (preferably less than about 0.5 nM) and/or a K_{D} for cyno TRDV1 of less than about 10 nM (preferably less than about 5 nM). Such antibodies may alternatively or additionally have an IC50 for TCR down regulation of less than about 1 nM (preferably less than about 0.5 nM).

Antibodies of or derived from ADT1-4-8 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-82 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-83 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-3 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-84 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-86 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM (preferably less than about 10 nM). Such antibodies may alternatively or additionally have an IC50 for TCR down regulation of less than about 1 nM (preferably less than about 0.5 nM).

Antibodies of or derived from ADT1-4-95 (for example fragments thereof, variants having one or more amino acid substitutions or having certain percent identity thereto) may have a K_{D} for human TRDV1 of less than about 10 nM (preferably less than about 1 nM) and/or a K_{D} for cyno TRDV1 of less than about 50 nM.

Antibodies of or derived from ADT1-4-1 (for example fragments thereof, variants having one or more amino acid substitutions or having certain percent identity thereto) may have a K_{D} for human TRDV1 of less than about 1 nM (preferably less than about 0.5 nM) and/or a K_{D} for cyno TRDV1 of less than about 10 nM (preferably less than about 1 nM). Such antibodies may alternatively or additionally have an IC50 for TCR down regulation of less than about 1 nM (preferably less than about 0.5 nM).

Antibodies of or derived from ADT1-4-6 (for example fragments thereof, variants having one or more amino acid substitutions or having certain percent identity thereto) may have a K_{D} for human TRDV1 of less than about 10 nM (preferably less than about 5 nM) and/or a K_{D} for cyno TRDV1 of less than about 10 nM (preferably less than about 1 nM). Such antibodies may alternatively or additionally have an IC50 for TCR down regulation of less than about 1 nM (preferably less than about 0.5 nM).

Antibodies of or derived from ADT1-4-138 (for example fragments thereof, variants having one or more amino acid substitutions or having certain percent identity thereto) may have a K_{D} for human TRDV1 of less than about 100 nM (preferably less than about 50 nM) and/or a K_{D} for cyno TRDV1 of less than about 100 nM (preferably less than about 50 nM). Such antibodies may alternatively or additionally have an IC50 for TCR down regulation of less than about 50 nM (preferably less than about 10 nM).

### Pharmacological Properties of Antibodies Comprising Fab Regions of the ADT1-7 Lineage

The pharmacological properties of the antibodies with respect to their binding (via the Fab region) to TRDV1 may fall within certain parameters. For example, antibodies of or derived from ADT1-7-10 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-15 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-17 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-18 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-19 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-20 (for example fragments thereof, variants having one or more amino acid substitutions or having certain percent identity thereto) may have a K_{D} for human TRDV1 of less than about 10 nM (preferably less than about 5 nM). Such antibodies may alternatively or additionally have an IC50 for TCR down regulation of less than about 5 nM (preferably less than about 1 nM).

Antibodies of or derived from ADT1-7-22 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-23 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-42 (for example fragments thereof, variants having one or more amino acid substitutions or having certain percent identity thereto) may have a K_{D} for human TRDV1 of less than about 10 nM (preferably less than about 5 nM).

Antibodies of or derived from ADT1-7-3 (for example fragments thereof, variants having one or more amino acid substitutions or having certain percent identity thereto) may have a K_{D} for human TRDV1 of less than about 10 nM (preferably less than about 1 nM). Such antibodies may alternatively or additionally have an IC50 for TCR down regulation of less than about 5 nM (preferably less than about 1 nM).

Antibodies of or derived from ADT1-7-61 (for example fragments thereof, variants having one or more amino acid substitutions or having certain percent identity thereto) may have a K_{D} for human TRDV1 of less than about 10 nM (preferably less than about 1 nM). Such antibodies may alternatively or additionally have an IC50 for TCR down regulation of less than about 50 nM (preferably less than about 10 nM).

### Other Functional Properties of the Antibodies

The antibodies of the present disclosure have an advantageous functional profile due to a number of their features.

The multispecific antibodies of the disclosure act to treat PD-L1 expressing cancers in a number of ways.

The primary mechanism of action of the multispecific Vδ1×PD-L1 antibodies of the disclosure is to engage Vδ1 γδ T cells and PDL1+ cancer cells, directing anti-tumor responses by enabling Vδ1 immunological synapse formation, Vδ1 TCR downregulation and subsequent Vδ1 γδ T cell tumor-targeted cytotoxicity through degranulation.

The multispecific Vδ1×PD-L1 antibodies of the disclosure also mediate activation of blood and tumor resident Vδ1 γδ cells, which drives Vδ1 γδ T cell proliferation as well as chemokine and cytokine release, demonstrating the potential for the antibodies of the disclosure to elicit a broader anti-tumor immune response.

One advantage of the multispecific Vδ1×PD-L1 antibodies of the disclosure is that they induce proliferation and activation of peripheral Vδ1 γδ T cells, partly due to the expression distribution of PD-L1 for example on normal immune cells. This includes blood resident Vδ1 γδ T cells even in the absence of adherent PD-L1⁺ cells. The multispecific Vδ1×PD-L1 antibodies of the disclosure alone (without any cytokines or endogenous growth agents or even tumour cells) are able to trigger activation and proliferation of Vδ1 γδ cells in a tumour free system.

The multispecific Vδ1×PD-L1 antibodies of the disclosure also prime the peripheral Vδ1 γδ T cells, for example by enhancing expression of cell surface activation markers, again in a tumour free environment without other external stimuli.

Another mechanism by which the multispecific Vδ1×PD-L1 antibodies of the disclosure exert an anti-tumour effect is by promoting tumour homing. The multispecific Vδ1×PD-L1 antibodies of the disclosure direct blood and tumor resident Vδ1 T cells to target PD-L1+ tumor cells. In the blood, the multispecific Vδ1×PD-L1 antibodies of the disclosure crosslink the Vδ1 TCR upon Vδ1 γδ T cells with PD-L1 expressed upon normal immune cells to stimulate Vδ1 γδ T cell proliferation and upregulation of key homing receptors, thereby promoting chemotaxis toward the tumor. In the tumor, the multispecific Vδ1×PD-L1 antibodies of the disclosure crosslink the Vδ1 TCR upon Vδ1 γδ T cells with PD-L1 expressed upon tumor cells, triggering Vδ1 γδ T cell activation and subsequent cytokine production, proliferation, and tumor-targeted cytotoxicity.

Additionally, the stimulation of Vδ1 T cells in the tumor mediated by the multispecific Vδ1×PD-L1 antibodies of the disclosure mediated activates non-Vδ1 immune cell subsets like natural killer (NK) cells and as T cells via secretion of immune stimulatory cytokines resulting in further antitumor activity mediated by non-Vδ1 lymphocytes.

This is outlined in more detail in Examples 18 to 23.

In addition, unlike anti-Vδ1 antibodies of the prior art which focus on depletion of Vδ1 T-cells, the antibodies of the present disclosure are useful for the activation of Vδ1 T-cells. Although they may cause downregulation of the TCRs on T-cells to which they bind, they do not cause Vδ1 T-cell depletion, but rather they stimulate the T-cells and hence may be useful in therapeutic settings that would benefit from the activation of this compartment of T-cells. Activation of Vδ1 T-cells is evident through TCR downregulation, changes in activation markers such as CD25 and Ki-67 and degranulation marker CD107a. Activation of Vδ1 T-cell in turn triggers release of inflammatory cytokines such as INFγ and TNFα to promote immune licensing. Surprisingly, antibodies having suitably high affinity for TRDV1 elicit increased Vδ1 T-cell killing and, unlike (for example) antibodies that target CD3, the provision of high affinity antibodies is possible without adverse effects associated with large-scale activation via CD3. In turn, the high affinity antibodies are able to induce strong immunostimulatory effects via tumour-infiltrating lymphocytes (TILs). This can be achieved with minimal exhaustion or killing of the Vδ1 cells. Therefore, the antibodies of the present disclosure may be considered agonistic antibodies.

In one embodiment of the disclosure, there is provided an anti-Vδ1 antibody or antigen-binding fragment thereof, characterised in that it:
a) causes downregulation of TCRs on Vδ1 T-cells;
b) does not exhibit CDC or ADCC; and
c) does not deplete Vδ1 T-cells.

In some embodiments, the multispecific anti-Vδ1 antibody or antigen-binding fragments also stimulate Vδ1 T-cell proliferation. This includes peripheral and/or blood-resident Vδ1 T-cell proliferation.

In some embodiments, multispecific antibodies of this disclosure confer one or more of
(a) enhanced TCR downregulation effects;
(b) enhanced Vδ1 T-cell proliferation effects; and/or
(c) enhanced Vδ1 T-cell activation effects
   upon Vδ1 T-cells when said cells are in the presence of (mixed with, proximal to, or are adjacent to) PD-L1+ve cells such as PD-L1+ cancer cells.

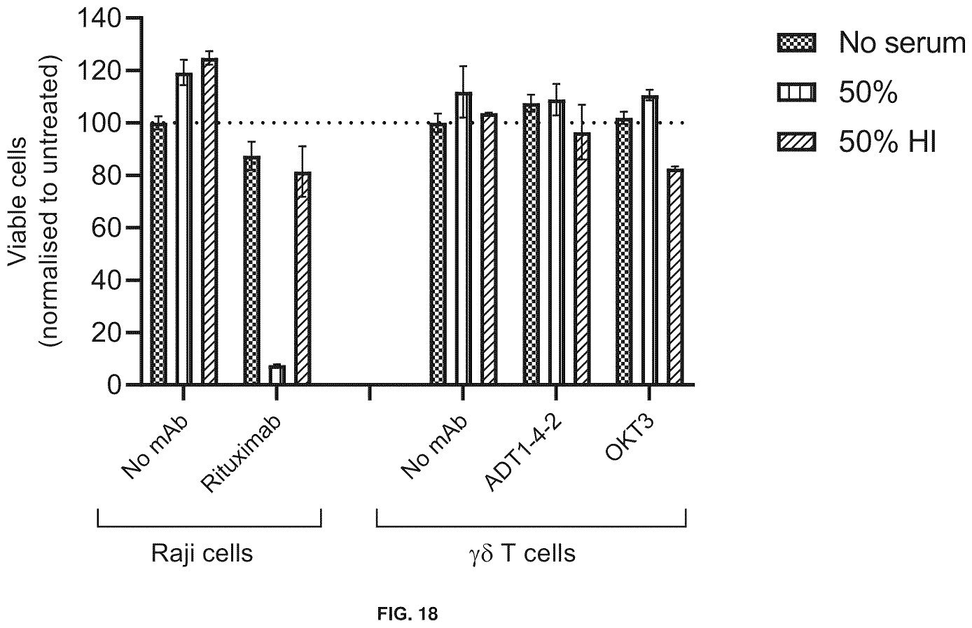

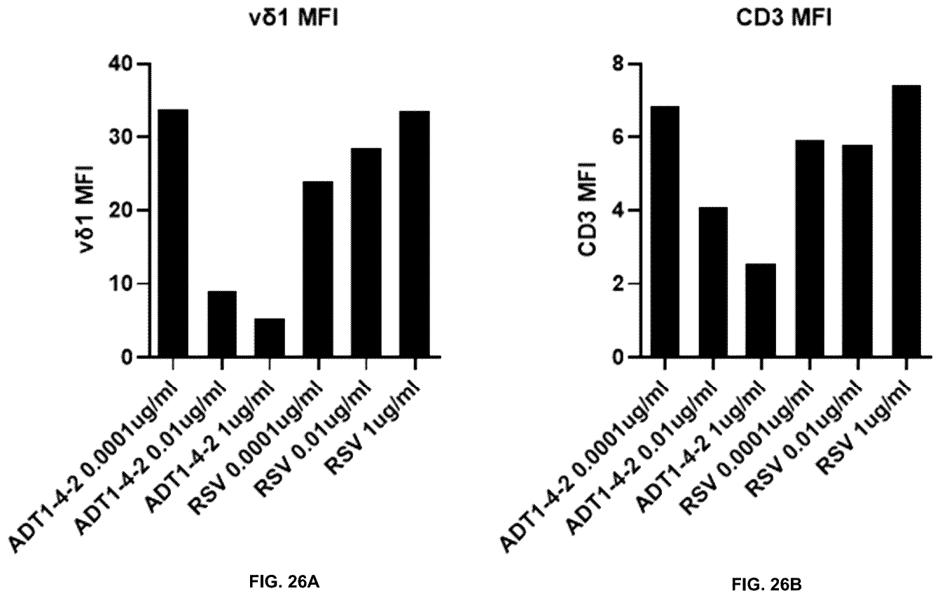

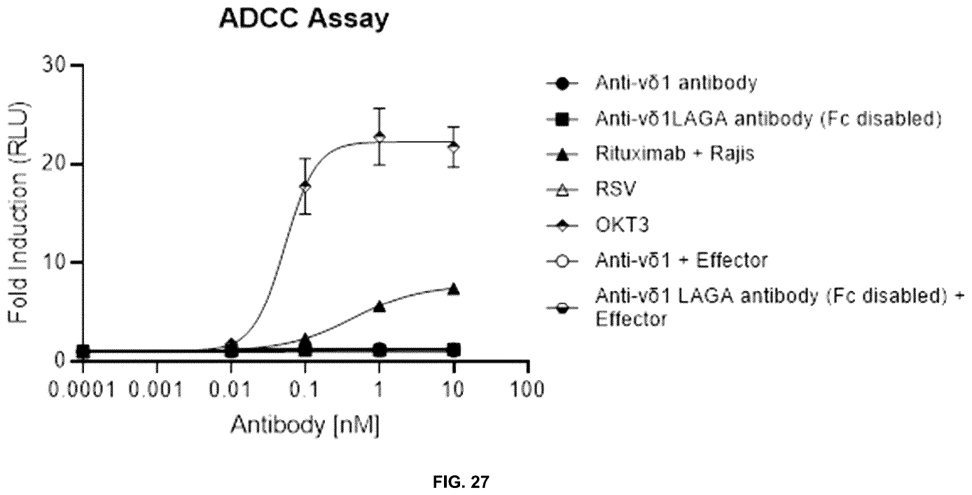

T-cell depletion is the process of T cell death removal or reduction. References to the antibodies or antigen binding fragments not depleting the Vδ1 T cells refers to a depletion of less than about 30% or less than about 20% (preferably less than about 10%) of the viable Vδ1 T+ cell population when incubated by one or more of the multispecific antibodies of the disclosure as described herein (for example when the antibodies is provided as an IgG1 antibody), and as measured by any via suitable means in a controlled study (for example via controlled flow cytometry methodology or via other established controlled assays such as described in FIG. 18 and FIGS. 26A-26B).

ADCC and CDC are mechanisms by which T-cell depletion may occur. Reference to the antibodies or antigen binding fragments herein not causing ADCC or CDC refers to a depletion of less than about 30% or less than about 20% (preferably less than about 10%) of the viable Vδ1 T+ cell population via ADCC and/or CDC when incubated by one or more of the multispecific antibodies of the disclosure as described herein (for example when the antibodies is provided as an IgG1 antibody), as measured by any via suitable means (for example via controlled flow cytometry methodology or via other established controlled assays such as described in FIG. 18).

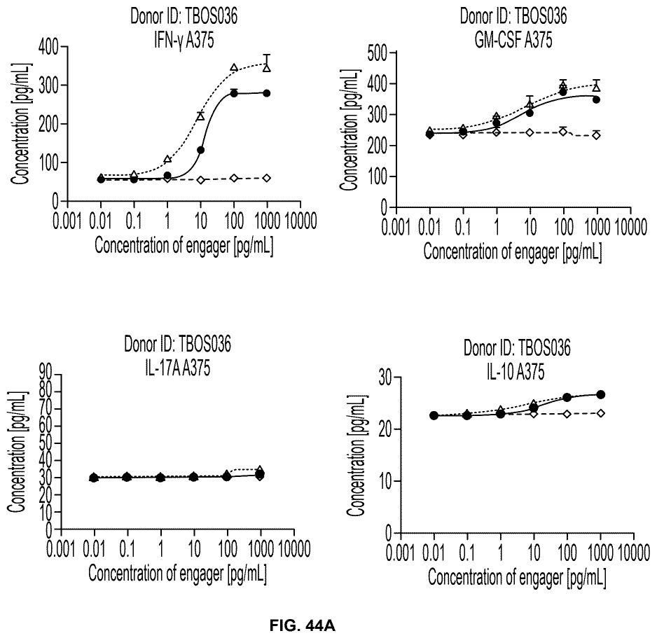

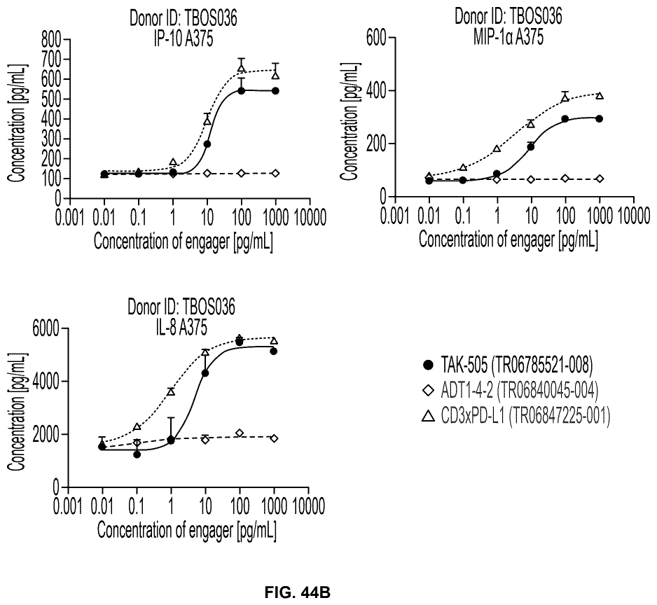

In one embodiment, there is provided an anti-Vδ1 antibody or antigen-binding fragment thereof, characterised in that it does not induce secretion of IL-17A. IL-17A (Interleukin-17A) is a pro-tumorigenic cytokine which is produced by activated T-cells. IL-17A can enhance tumour growth and dampen the anti-cancer immune response. As shown in FIGS. 44A-44B, anti-vδ1 antibodies do not induce secretion of IL-17A. Hence, reference to the antibodies or antigen binding fragments herein not inducing secretion of IL-17A refers to inducing less than about 30%, or less than about 20%, or less than about 10% of the IL-17A secretion induced by comparator anti-CD3 antibodies in such circumstances (for example as typified by OKT 3; an anti-CD3 comparator).

### Antibody Modifications

The antibodies and fragments thereof may be modified in other ways using known methods. Sequence modifications to antibody molecules described herein can be readily incorporate by those skilled in the art. The following examples are non-limiting.

During antibody discovery and sequence recovery from phage libraries, desired antibody variable domains may be re-formatted into full length IgG by sub-cloning. To accelerate the process, variable domains are often transferred using restriction enzymes. These unique restriction sites may introduce additional/alternate amino acids and away from the canonical sequence (such canonical sequences may be found, for example, in the international ImMunoGeneTics [IMGT] information system, see imgt.org). These may be introduced as kappa or lambda light chain sequence modifications.

### Light Chain Modifications

The variable light chain variable sequences may be cloned using restriction sites (e.g. Nhe1-Not1) during re-formatting into full length IgG. More specifically, at the light chain N-terminus, an additional Ala-Ser sequence was introduced in the parental (non-affinity matured) antibodies to support cloning. Preferably, this additional AS sequence is then removed during further development such to generate the canonical N-terminal sequence. Hence, in some embodiments, light chain containing antibodies described herein do not contain an AS sequence at their N-termini, i.e. SEQ ID NOs: 26, 118, 282 to 290 or 313 do not comprise the initial AS sequence. The N-termini of the light chain sequences of the affinity-matured antibodies already do not comprise this AS motif.

Additional amino acid changes may be made to support cloning. For example, for the parental antibodies described herein having kappa light chains (i.e. B07, C05, E04, F07, G06, G09, B09, G10, G04 and E07), at the kappa light-chain variable-domain/constant domain border a valine-to-alanine change was introduced to support cloning when preparing full-length sequences. This resulted in a kappa constant domain modification. Specifically, this results in the constant domain beginning RTAAAPS (SEQ ID NO: 689) (from a NotI restriction site). Preferably, such sequences can be modified during further development to generate the canonical kappa light-chain constant regions which start with RTVAAPS (SEQ ID NO: 690). Such modifications do not change the functional properties of the antibodies. Hence, in some embodiments, kappa light chain containing antibodies described herein contain a constant domain starting with the sequence RTV (for example as in SEQ ID NO: 296).

As another example, for the antibodies described herein (specifically E01 and C08) at the lambda light-chain variable-domain/constant domain border a lysine-to-alanine sequence change was introduced to support cloning. This resulted in a lambda constant domain modification. Specifically, this results in the constant domain beginning with GQPAAAPS (SEQ ID NO: 691) (from a NotI restriction site). Preferably, this sequence can be modified during further development such to generate the canonical lambda light constant region which starts GQPKAAPS (SEQ ID NO: 692). Hence, in some embodiments, lambda light chain containing antibodies described herein contain a constant domain starting with the sequence GQPK (SEQ ID NO: 693).

### Heavy Chain Modifications

The “Fcab” CH3 modifications necessary to achieve PD-L1 binding are described in more detail elsewhere.

Typically, human variable heavy chain sequences start with either the basic glutamine (Q) or acidic glutamate (E). However, both such sequences are then known to convert to the acidic amino acid residue, pyroglutamate (pE). The Q to pE conversion results in a charge change to the antibody, whilst an E to pE conversion does not change the charge of the antibody. Hence to avoid a variable charge-change over time one option is to modify a starting heavy chain sequence from Q to E in the first instance. Hence, in one embodiment, the heavy chain of antibody described herein having a Q residue at the N-terminus of the heavy chain may contain a Q to E modification at the N-terminus. In particular, the initial residue of any of SEQ ID NOs: 1, 106, 276 to 279 or 312 may be modified from Q to E. It will be understood that this embodiment also applies to any embodiment incorporating these sequences, for example into full-length multispecific antibodies or antigen-binding fragments thereof. In some embodiments, it may be advantageous to substitute an E residue at the N-terminus of the heavy chain to an E residue. Accordingly, in some embodiments, the E residue at the N-terminus of any one SEQ ID NOs: 2 to 25, 107 to 117, 273 to 275, 280 or 281 may be substituted with a Q residue.

Furthermore, the C-terminus of the IgG1 constant domain ends with PGK. However, the terminal basic lysine (K, EU position 447) is then often cleaved during expression (e.g. in CHO cells). This in turn results in charge change to the antibody through varied loss of the C-terminal lysine residue. Therefore, one option is to remove the lysine in the first instance resulting in a uniform and consistent heavy chain C-terminus sequence ending in PG. An alternative option is to also remove the terminal G (EU position 446). Hence, in one embodiment, the heavy chain of an antibody described herein has the terminal K, or the terminal GK, removed from its C-terminus.

In some embodiments, the antibody or antigen-binding fragment thereof contains a modified effector function through alteration to the sugars linked to Asn 297 (EU numbering scheme). In a further said modification, Asn 297 is not fucosylated or exhibits reduced fucosylation (i.e., a defucosylated antibody or a non-fucosylated antibody). Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. The antibody may be modified to prevent or inhibit fucosylation of the antibody. Typically, glycosylation modifications involve expressing said antibody or antigen-binding fragment thereof in a host cell containing alternate glycosylation processing capabilities either through targeted engineering or through targeted or serendipitous host or clone selection (e.g. see Example 13). These and other effector modifications are discussed further in recent reviews such as by Xinhua Wang et al. (2018) Protein & Cell 9: 63-73 and by Pereira et al. (2018) mAbs 10(5): 693-711 and which are hereby incorporated.

### Optional Allotype Modifications

During antibody discovery, specific human allotypes may be employed. Optionally, the antibodies can be switched to differing human allotypes during development. By way of non-limiting example, for the kappa chain there are three human allotypes designated Km1, Km1,2 and Km3 which define three Km alleles (using allotype numbering): Km1 correlates with valine 153 (IMGT V45.1) and leucine 191 (IMGT L101); Km1,2 correlates with alanine 153 (IMGT A45.1) and leucine 191 (IMGT L101); and Km3 correlates with alanine 153 (IMGT A45.1) and valine 191 (IMGT V101). Optionally, one can therefore modify a sequence from one allotype to another by standard cloning approaches. For example, a L191V (IMGT L101V) change will convert a Km1,2 allotype to a Km3 allotype. For further reference on such allotypes see Jefferis and Lefranc (2009) MAbs 1(4):332-8, which is herein incorporated by reference.

Hence in one embodiment an antibody described herein contains amino acid substitutions derived from another human allotype of the same gene. In a further embodiment, the antibody contains a L191V (IMGT L101V) substitution to the kappa chain to convert the c-domain from a km1,2 to a km3 allotype.

### Multispecific Antibodies

The antibodies of the present disclosure are multispecific. They may be bispecific. Multispecific antibodies may be specific for different epitopes of one target polypeptide or may be specific for more than one target polypeptide. Therefore, in one embodiment, the antibody or antigen-binding fragment thereof comprises a first binding specificity for Vδ1 and a second binding specificity for PD-L1.

In particular, the present disclosure is a member of a new class of high-affinity antibodies that comprise multiple antigen-binding sites (“multispecific antibodies”), including an antigen-binding site for TCR delta variable 1 (Vδ1) and an antigen-binding site for PD-L1.

The multispecific antibodies of the present disclosure are in the format known as “mAb² antibodies” or “mAb squared antibodies”, which are antibodies comprising an Fc region that has been engineered to contain antigen-binding loops in its CH3 domain—this modified Fc region is termed an “Fcab”. The mAb² antibody further comprises a Fab region, comprising a VH-VL domain pair providing an antigen-binding site. mAb² molecules of the present disclosure comprise a PD-L1 binding Fcab and an vδ1-binding Fab.

The TCEs of the present disclosure provide several advantages over the TCEs of the prior art. In particular, the TCEs of the present disclosure are able to treat PD-L1 expressing cancers in a number of ways by virtue of specifically binding both Vδ1 and PD-L1, for example:
by engaging Vδ1 γδ T cells and PDL1+ cancer cells, directing anti-tumor responses by enabling Vδ1 immunological synapse formation, Vδ1 TCR downregulation and subsequent Vδ1 γδ T cell tumor-targeted cytotoxicity through degranulation.
by mediating activation of blood and tumor resident Vδ1 γδ cells, which drives Vδ1 γδ T cell proliferation as well as chemokine and cytokine release. This further demonstrates the potential for the antibodies of the disclosure to elicit a broader anti-tumor immune response.
by inducing proliferation and activation of peripheral Vδ1 γδ T cells, partly due to the expression distribution of PD-L1 for example on normal immune cells. This includes blood resident Vδ1 γδ T cells even in the absence of adherent PD-L1+ cells. The multispecific Vδ1×PD-L1 antibodies of the disclosure alone (without any cytokines or endogenous growth agents or even tumour cells) are able to trigger activation and proliferation of Vδ1 γδ cells in a tumour free system.
by priming the peripheral Vδ1 γδ T cells, for example by enhancing expression of cell surface activation markers, again in a tumour free environment without other external stimuli.
by promoting tumour homing. The multispecific Vδ1×PD-L1 antibodies of the disclosure direct blood and tumor resident Vδ1 T cells to target PD-L1+ tumor cells. In the blood, the multispecific Vδ1×PD-L1 antibodies of the disclosure crosslink the Vδ1 TCR upon Vδ1 γδ T cells with PD-L1 expressed upon normal immune cells to stimulate Vδ1 γδ T cell proliferation and upregulation of key homing receptors, thereby promoting chemotaxis toward the tumor. In the tumor, the multispecific Vδ1×PD-L1 antibodies of the disclosure crosslink the Vδ1 TCR upon Vδ1 γδ T cells with PD-L1 expressed upon tumor cells, triggering Vδ1 γδ T cell activation and subsequent cytokine production, proliferation, and tumor-targeted cytotoxicity.
by activating non-Vδ1 immune cell subsets like natural killer (NK) cells and αβ T cells via secretion of immune stimulatory cytokines resulting in further antitumor activity mediated by non-Vδ1 lymphocytes.

In addition, the TCEs may overcome many of the challenges associated with TCEs of the prior art by specifically targeting (and activating) the T-cell receptor complex solely via binding to an epitope on the TRDV1 domain. In this manner a number of advantages are realized including:
Engaging only a sub-set of T-cells rather than all T-cells (e.g. engagement of T-regs in a cancer setting may be undesirable);
Engaging a sub-set of T-cells (TRDV1+ T-cells) that are predominantly ‘tissue-resident’ and whose presence often positively correlates with good prognosis in a cancer/tumour setting;
Activating the T-cell receptor complex via TRDV1 engagement thereby affording more optionality (e.g. increasing affinity of this binding domain, as in the antibodies of the present disclosure). For example, by developing recombinant TCEs which engage a T-Cell Receptor complex solely via the TRDV1 domain rather than via CD3, increased affinity may drive more favourable functionality. For example, high-affinity TRDV1-binding TCEs may activate but not exhaust T-cells; and/or
Engaging the TCE complex via said novel means and via recombinant TRDV1 binding domains may result in less deleterious effects and so reduce the need to attenuate Fc functionality in said TCE moieties. It would generally be expected that a TCE with unattenuated Fc function would induce an antibody-dependent cell-mediated cytotoxicity (ADCC) effect and thereby deplete the population of γδ T-cells recognized by the antibody. However, and again, by attenuating such functionality to avoid toxicity/safety complexities, one may also attenuate a potentially important efficacy angles too e.g. by engaging CD16+ or CD32+ or CD64+ immune cells, or by reducing half-life of the bispecific (e.g. if employing smaller bispecific antibody fragments such as BITEs). Methods of reducing the interaction of the FcγR and the TCE (such as using an IgG format designed to reduce said interaction) would be expected to reduce Fc-mediated immobilization of the TCE and reduce TCR clustering by cross-linking with the immobilized TCE. Reducing the need to attenuate Fc functionality in said TCE moieties then may afford additional optionality, for example by allowing TRDV1 TCEs to engage TRDV1+ cells via one binding domain, engaging a second cell type (such as a cancerous cell) by a second binding arm, and engaging other effector cells such as CD16+ or CD32+ or CD64+ immune cells via a functioning Fc domain.
Activate the vδ1 TCR without depleting vδ1+ T cells. The multispecific antibodies described herein can therefore engage and activate the CD3/γδ TCR complex and confer its downregulation and loss of surface expression, without unwanted depletion of vδ1 T cells (see FIGS. 26A-26B). This enables the multispecific antibodies described herein to be employed as medicaments to treat a disease or disorder such to ameliorate at least one sign or symptom of a disease or disorder through a mechanism involving the activation of blood, tissue and tumour resident Vδ1+ T cells.

Finally, the “mAb²” format (comprising the modified PD-L1 binding “Fcab”) also conveys advantageous properties. For example:
The anti-PD-L1 Fcabs of the disclosure exhibited similar potency in the PD-1/PD-L1 reporter assay and their potency was unchanged regardless of different anti-Vδ1 Fab sequences. They are also able to bind to and inhibit PD-L1 with similar affinity and potency as a monoclonal antibody benchmark despite the two antigen binding sites of an Fcab being spatially close to each other (as compared with those of a typical mAb). Based on this smaller binding interface and reduced flexibility of the two binding sites it is surprising that the multispecific antibodies of the disclosure (comprising Fcab regions) are able to retain their potency.
The anti-PD-L1 Fcabs are stable and do not aggregate. Antibody structures and stability can be unpredictable, and this is especially true for multispecific formats that attempt to incorporate multiple binding domains. The Fcabs of the disclosure help retain the IgG like format/structure whilst also offering some degree of conditional activation via avidity.

Specifically, the antibodies of the disclosure engage the T-cell receptor via a TRDV1 domain rather than other domains in said T-cell receptor signalling complexes. More specifically the antibodies of the disclosure engage this complex via an activating epitope on TRDV1 and which may be bound at higher affinities without potentially conferring some of the previously reported deleterious effects high-affinity T-cell receptor complex engagement. Further this class of TCEs may engage in such a way which may allow for wild-type Fc functionality too, thereby affording additional efficacy potential too.

The multispecific antibodies of the disclosure may also display improved properties compared to equivalent monospecific antibodies. For example, the multispecific antibodies of the disclosure may also display improved properties compared to monospecific antibodies having the same antigen binding domains as the component parts of the multispecific antibodies. In some embodiments, for example, the recombinant multispecific antibody confers increased gamma delta T-cell mediated cytotoxicity towards a diseased cell expressing the second epitope compared to the cytotoxicity conferred by an equivalent amount of said first monospecific antibody. The multispecific antibodies of the disclosure may also display improved cytotoxicity towards diseased cells whilst still sparing healthy cells.

In some embodiments the multispecific antibody is a human recombinant antibody encoded by a recombinant nucleic acid open reading frame or frames expressed from a recombinant host cell. In some embodiments the multispecific antibody is not a rodent or other non-human antibody derived from B-cell fusion hybridoma technologies. In some embodiments the multispecific antibody does not comprise non-human IgG constant domain sequence found only in non-human animal species, such as sequence found in rodent-derived hybridomas.

In preferred embodiments, the multispecific antibodies (suitably bispecific antibodies) of the disclosure, do not specifically bind (or directly interact with) CD3.

References herein to an antigen being “on” a cell refer to antigens that are expressed on the cell surface membrane or are associated with the (extracellular side of) the cell surface membrane of such cells.

The second target epitope is PD-L1. PD-L1 is an immunomodulatory antigen and an example of an immune checkpoint inhibitor. PD-L1 is usually expressed on a range of normal immune cells (monocytes, granulocytes, lymphocytes) in the periphery, playing an important role in immune-homeostasis through suppressing immune cell activation via its interaction with programmed cell death protein 1. PD-L1 is also expressed by tumour cells as a mechanism to escape anti-tumour responses since the binding of PD-L1 to its receptor PD-1 results in inhibition of T cell activation. PD-L1 is therefore an established tumor target that is upregulated in a number of solid tumor indications including head and neck squamous cell carcinoma (HNSCC), non-small cell lung cancer (NSCLC), colorectal cancers (CRC) and gastric cancers, and is associated with immune evasion and disease progression. In cancer settings this results in immune escape for tumor cells by inactivating cytolytic CD8+ T-cells. Blockade of PD-L1 is therefore a promising therapeutic target. Monoclonal antibodies that specifically bind to epitopes of PD-L1 are well known in the art. For example, durvalumab and atezolizumab are monoclonal antibody which binds specifically to an epitope of PD-L1.

### Healthy Cell Sparing

While the mechanisms by which γδ T-cells recognize antigens and distinguish between healthy and diseased cells are not fully understood (Ming Heng and Madalene Heng, Antigen Recognition by γδ T-Cells. Madame Curie Bioscience Database [Internet], Austin (TX): Landes Bioscience; 2000-2013), the fact that γδ T-cells are able to distinguish between healthy cells and diseased cells and exhibit remarkable diseased cell polycytotoxicity means that they can be leveraged to provide improved medicaments with improved therapeutic windows. Further, by leveraging such γδ T-cell capabilities, there is provided an opportunity to treat disease while sparing healthy cells, by colocalizing γδ T-cells with diseased cells even when a particular cancer antigen, inflammatory antigen, or pathogen antigen is either not known, or is also present on healthy cells, in a particular patient.

One of the challenges of current or conventional approaches is less favorable toxicity profiles. This is because the PD-L1 antigen is expressed on healthy tissues such as epithelial cells as well as on cancerous cells. Hence use of anti-PD-L1 medicaments which engage and co-localize all T-cells with PD-L1 positive cells can result in less favorable therapy windows or therapeutic indices. This is because such medicaments will engage all T-cells of which the vast majority in circulation will be as T-cells (CD4+ positive, CD8+ positive etc.). And once as T-cells are co-localized with PD-L1 positive cells, such conventional as T-cells exhibit limited capabilities to spare PD-L1+ healthy cells and limited capabilities to kill only diseased PD-L1+ diseased cells.

Hence instead of employing such conventional approaches, provided herein are multispecific antibodies wherein at least one first binding domain is able to specifically bind Vδ1+ cells and at least one second binding domain is able to specifically bind to PD-L1, a target present on diseased tissues and cells. The use of such multispecific antibodies in this way may thereby result in the co-localization of Vδ1+ cells to diseased cells expressing the second target. Further, and given such disease associated targets are not often 100% disease specific, this approach of targeting and co-localizing Vδ1+ effector cells specifically, may be more preferred over conventional approaches. This is because Vδ1+ effector cells may be capable of recognizing stress patterns in diseased or infected cells and so may be able to selectively kill diseased cells whilst sparing healthy cells also expressing the same target. Going beyond this, the multispecific antibodies of the disclosure are surprisingly also able to trigger activation and proliferation of Vδ1 γδ cells in a tumour free system (e.g. without any cytokines or endogenous growth agents or even tumour cells). They are able to induce proliferation and activation of peripheral Vδ1 γδ T cells, and further act to prime the peripheral Vδ1 γδ T cells for example by enhancing expression of cell surface activation markers, again in a tumour free environment without other external stimuli. This is partly due to the expression distribution of PD-L1 (for example on normal immune cells), which enables a potential disadvantage (side effects on healthy cells) to be turned to an advantage.

The multispecific antibodies presented herein are therefore able to engage on the TCR of vδ1 cells and trigger proliferation and priming of said vδ1 cells, but full activation does not occur unless tumour cells are also present. Full engagement of the presently presented antibodies on the TCR leads to partial downregulation and it is believed the vδ1 cells bound by the presently presented antibodies only become fully activated and become cytotoxic when in the presence of stressed cells such as tumour cells. This is shown, for example, in Example 15). This represents another vital safety advantage for the approach presented herein, since off target cytotoxicity is reduced and the full potency of the multispecific antibodies to activate vδ1 cells is only unleashed in the presence of tumour cells, meaning healthy cells (even healthy cells expressing PD-L1) are spared.

One mechanism behind γδ T cells being able to detect stress signals on tumour cells is believed to be due to the NCRs (natural cytotoxicity receptors) they express. The NCRs are able to engage NCR ligands on tumour cells. A dual mechanism of activation may therefore be employed, wherein the γδ T cells are activated via TCR stimulation, including via NCRs, which can sense the tumour cells to enable full activation and cytotoxicity.

This contrasts with stimulation of αβ T cells via CD3, for example, wherein all stimulation is via the TCR. Such cells are therefore almost indiscriminate between healthy or transformed cells because they do not have mechanisms such as antigen presentation independent sensing of tumour cells, for example via NCRs. Therefore, if CD3 antibodies are Fc enabled they will attract other immune cells which can trigger a cascade of unpredictable and desirable events such as cytokine storms, exhaustion and even overactivation of immune cells leading to, for example, NK cells killing T cells etc. In the present approach, stimulation of γδ T cells with the presently presented multispecific antibodies do not lead to such concerns because γδ T cells) are able to distinguish between healthy cells and tumour cells, including via their NCR sensing mechanism and therefore selectively kill stressed cells such as cancer cells or virally infected cells due to this diseased cell specificity.

The second target epitope may be on a different cell including a different T-cell, a B-cell, a tumour cell, an immune cell (such as a monocyte, a granulocyte or a lymphocyte), an autoimmune tissue cell or a virally infected cell.

Various antibody-derived multispecific formats have been described previously and are typically built empirically from the component binding parts. Typically, once constructed, the performance of such multispecific or multi-target binding formats as described herein may be measured in one or more of the aforementioned model systems (cell killing, cell proliferation, healthy cell sparing/diseased cell specific models etc). Optionally they are also compared to said component parts and other comparator molecules.

Whilst not being limited by this approach, in general when constructing antibodies as multispecific antibodies of this disclosure, the binding domain modules to each target (first and second) are built from Fab and Fcab binding modules which are combined into full length antibodies. Modification and generation of multispecific antibodies and desired Fab and Fcab binding domains can be undertaken by routine molecular biology methodology well known in the art. By way of none-limiting examples, oligonucleotide-based approaches with or without aid of PCR amplification can be employed to build synthetic DNA sequences encoding a desired CH3 domain and/or other elements of the desired bispecific antibody. Optionally these can then be correctly cloned into an open reading frame (ORF) encoding the starting antibody heavy chain (such as one described herein) via restriction enzyme based sub-cloning methodologies such to replace the starting CH3 sequence with the desired modified CH3 sequence. Reciprocally, one can employ oligonucleotide-based approaches with or without aid of PCR amplification to generate DNA ORF elements designed to encode the desired VH region (such as one described herein) and then sub-clone or switch this into a pre-existing heavy chain ORF cassette already expressing the desired CH3 domain encoding region. Alternatively, larger elements or indeed the entire antibody heavy chain encoding ORF can be built de-novo from a starting in-silico sequence with oligonucleotide and/or PCR methodologies to generate the entire final ORF encoding a preferred full-length heavy chain comprising a VH binding arm and modified CH3 domain of the multispecific antibody such as described herein. This can then be expressed, recovered and purified in combination with a preferred cognate kappa light chain such as that described herein in order to generate a final purified, multispecific antibody which binds the TRDV1 domain of human Vδ1 TCR and is operatively linked to a modified CH3 binding domains which bind human PD-L1.

Remarkably, multispecific antibodies comprising at least one (first) binding domain targeting the Vδ1 chain of a γδ TCR as described herein are further enhanced when said first binding domain is formatted with a multispecific antibody format comprising at least one second binding domain against PD-L1.

### Multispecific Antibodies—Non-Limiting Examples:

To outline the applicability of the approach a series of non-limiting example multispecific antibodies were constructed. These multispecific antibodies comprised at least one (first) binding domain targeting the Vδ1 chain of a γδ TCR and at least one (second) binding domain targeting PD-L1:

### ADT5-1n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 564 | PD-L1 ADT5-1n CH3 binding module |
| 565 | PD-L1 ADT5-1n AB substitutions (EU numbering) |
| 566 | PD-L1 ADT5-1n CD substitutions (EU numbering) |
| 567 | PD-L1 ADT5-1n EF substitutions (EU numbering) |
| 585 | PD-L1 ADT5-1n Binding Module CH1-CH2-CH3 (LAGA) |
| 586 | PD-L1 ADT5-1n Binding Module CH1-CH2-CH3 (wt) |
| 587 | ADT1-4-2 HC PD-L1 ADT5-1n LAGA |
| 588 | ADT1-4-2 HC PD-L1 ADT5-1n WT |
| 589 | ADT1-4-2 × ADT5-1n (LAGA) |
| 590 | ADT1-4-2 × ADT5-1n (WT) |
| 591 | ADT1-4-1 HC PD-L1 ADT5-1n LAGA |
| 592 | ADT1-4-1 HC PD-L1 ADT5-1n WT |
| 593 | ADT1-4-1 × ADT5-1n (LAGA) |
| 594 | ADT1-4-1 × ADT5-1n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 565, the second sequence has the sequence set forth in SEQ ID NO: 566, and the third sequence has the sequence set forth in SEQ ID NO: 567, and the residue at position 433 of the CH3 domain is arginine (R).

In this aspect, the IgG1 CH3 domain may comprise the following mutations (EU numbering). In the AB loop the following mutations may be present: L358˜.K360G.N361Y.Q362W (˜ denotes an amino acid deletion). In the CD loop the following mutations may be present: Q386E.E388Q.N389Y.N390W.Y391A. In the EF loop the following mutations may be present: D413S.K414N.S415W.Q419L.N421 D. The IgG1 CH3 domain may also comprise the following mutation outside of the structural loop regions: H433R.

In this aspect the amino acid sequence of the CH3 domain may be at least 90% identical to, or at least 95% identical to, or 100% identical to SEQ ID NO: 564.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 585 or SEQ ID NO: 586.

In this aspect the Fab region may be any of the Fab regions described herein (see Tables 9 and 10, for example).

TABLE 9
ADT5-1n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 567 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 565 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

In some embodiments, the multispecific antibodies of the disclosure may have one or both of the two residues at the immediate C-terminus of the CH3 domain sequence removed (G446 and/or K447, EU numbering).

TABLE 10
ADT5-1n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to 362 = | SEQ ID | SEQ ID |
| ADT1-4-110 | 4 | 29 | SEQ ID | NO: 566 | NO: 567 |
| ADT1-4-112 | 5 | 30 | NO: 565 | | |
| ADT1-4-117 | 6 | 31 | | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-1 n

For example, referring to Table 9, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 565, optionally SEQ ID NO: 566, and SEQ ID NO: 567.

For example, referring to Table 10, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 565, optionally SEQ ID NO: 566, and SEQ ID NO: 567.

Every row of the above tables is disclosed in the same manner. Such antibodies are human IgG1 antibodies. Therefore, the human IgG1 antibodies may comprise a CH3 domain comprising the sequence of SEQ ID NO:564.

In this aspect, the multispecific antibody may comprise SEQ ID NO: 589; or SEQ ID NO: 414 and SEQ ID NO: 587. In this aspect, the multispecific antibody may comprise SEQ ID NO: 590; or SEQ ID NO: 414 and SEQ ID NO: 588.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 564.

### ADT1-4-1×ADT5-1n

For example, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 565 optionally SEQ ID NO: 566, and SEQ ID NO: 567.

For example, referring to Table 10, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 565, optionally SEQ ID NO: 566, and SEQ ID NO: 567.

Every row of the above table is disclosed in the same manner. Such antibodies are human IgG1 antibodies. Therefore, the human IgG1 antibodies may comprise a CH3 domain comprising the sequence of SEQ ID NO: 564.

In this aspect, the multispecific antibody may comprise SEQ ID NO: 593; or SEQ ID NO: 685 and SEQ ID NO: 591. In this aspect, the multispecific antibody may comprise SEQ ID NO: 594; or SEQ ID NO: 685 and SEQ ID NO:592.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 564.

### ADT5-2n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 568 | PD-L1 ADT5-2n CH3 binding module |
| 569 | PD-L1 ADT5-2n AB substitutions |
| 566 | PD-L1 ADT5-2n CD substitutions |
| 567 | PD-L1 ADT5-2n EF substitutions |
| 595 | PD-L1 ADT5-2n Binding Module CH1-CH2-CH3 |
| | (LAGA) |
| 596 | PD-L1 ADT5-2n Binding Module CH1-CH2-CH3 |
| | (wt) |
| 597 | ADT1-4-2HC PD-L1 ADT5-2n LAGA |
| 598 | ADT1-4-2HC PD-L1 ADT5-2n WT |
| 599 | ADT1-4-2 × ADT5-2n (LAGA) |
| 600 | ADT1-4-2 × ADT5-2n (WT) |
| 601 | ADT1-4-1HC PD-L1 ADT5-2n LAGA |
| 602 | ADT1-4-1HC PD-L1 ADT5-2n WT |
| 603 | ADT1-4-1 × ADT5-2n (LAGA) |
| 604 | ADT1-4-1 × ADT5-2n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 569, the second sequence has the sequence set forth in SEQ ID NO: 566, and the third sequence has the sequence set forth in SEQ ID NO: 567.

In this aspect, the IgG1 CH3 domain may comprise the following mutations (EU numbering). In the AB loop the following mutations may be present: L358˜.T359E.K360G.N361Y.Q362W (˜ denotes an amino acid deletion). In the CD loop the following mutations may be present: Q386E.E388Q.N389Y.N390W.Y391A. In the EF loop the following mutations may be present: D413S.K414N.S415W.Q419L. N421 D.

In this aspect the amino acid sequence of the CH3 domain may be at least 90% identical to, or at least 95% identical to, or 100% identical to SEQ ID NO: 568.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 595 or SEQ ID NO: 596.

In this aspect the Fab region may be any of the Fab regions described herein (see Tables 9 and 10, for example).

TABLE 11
ADT5-2n - Example multispecific vδ1-PD-L1 human lgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 567 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 569 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 12
ADT5-2n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 567 |
| ADT1-4-112 | 5 | 30 | SEQ ID | | |
| ADT1-4-117 | 6 | 31 | NO: 569 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-2n

For example, referring to Table 11, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 569, optionally SEQ ID NO: 566, and SEQ ID NO: 567.

For example, referring to Table 12, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 569, SEQ ID NO: 566, and SEQ ID NO: 567.

Every row of the above tables is disclosed in the same manner. Such antibodies are human IgG1 antibodies. Therefore, the human IgG1 antibodies may comprise a CH3 domain comprising the sequence of SEQ ID NO: 568.

In this aspect, the multispecific antibody may comprise SEQ ID NO: 599; or SEQ ID NO: 414 and SEQ ID NO: 597. In this aspect, the multispecific antibody may comprise SEQ ID NO: 600; or SEQ ID NO: 414 and SEQ ID NO: 598.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 568.

### ADT1-4-1×ADT5-2n

For example, referring to Table 11, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 569, SEQ ID NO: 566, and SEQ ID NO: 567.

For example, referring to Table 12, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 569, optionally SEQ ID NO: 566, and SEQ ID NO: 567.

Every row of the above table is disclosed in the same manner. Such antibodies are human IgG1 antibodies. Therefore, the human IgG1 antibodies may comprise a CH3 domain comprising the sequence of SEQ ID NO: 568.

In this aspect, the multispecific antibody may comprise SEQ ID NO: 603; or SEQ ID NO: 685 and SEQ ID NO: 601. In this aspect, the multispecific antibody may comprise SEQ ID NO: 604; or SEQ ID NO: 685 and SEQ ID NO: 602.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 568.

### ADT5-3n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 570 | PD-L1 ADT5-3 CH3 binding module |
| 571 | PD-L1 ADT5-3 AB substitutions |
| 566 | PD-L1 ADT5-3 CD substitutions |
| 572 | PD-L1 ADT5-3 EF substitutions |
| 605 | PD-L1 ADT5-3n Binding Module CH1-CH2-CH3 |
| | (LAGA) |
| 606 | PD-L1 ADT5-3n Binding Module CH1-CH2-CH3 |
| | (wt) |
| 607 | ADT1-4-2HC PD-L1 ADT5-3n LAGA |
| 608 | ADT1-4-2HC PD-L1 ADT5-3n WT |
| 609 | ADT1-4-2 × ADT5-3n (LAGA) |
| 610 | ADT1-4-2 × ADT5-3n (WT) |
| 611 | ADT1-4-1HC PD-L1 ADT5-3n LAGA |
| 612 | ADT1-4-1HC PD-L1 ADT5-3n WT |
| 613 | ADT1-4-1 × ADT5-3n (LAGA) |
| 614 | ADT1-4-1 × ADT5-3n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 571, the second sequence has the sequence set forth in SEQ ID NO: 566, and the third sequence has the sequence set forth in SEQ ID NO: 572.

In this aspect, the IgG1 CH3 domain may comprise the following mutations (EU numbering). In the AB loop the following mutations may be present: L358˜.T359S.K360G.N361Y.Q362W (˜ denotes an amino acid deletion). In the CD loop the following mutations may be present: Q386E.E388Q.N389Y.N390W.Y391A. In the EF loop the following mutations may be present: D413S.K414N.S415W. Q419L.G420D.N421 D.V422˜.

In this aspect the amino acid sequence of the CH3 domain may be at least 90% identical to, or at least 95% identical to, or 100% identical to SEQ ID NO: 570.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 605 or SEQ ID NO: 606.

In this aspect the Fab region may be any of the Fab regions described herein (see Tables 13 and 14, for example).

TABLE 13
ADT5-3n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 572 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 571 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 14
ADT5-3n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 572 |
| ADT1-4-112 | 5 | 30 | SEQ ID | | |
| ADT1-4-117 | 6 | 31 | NO: 571 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-3n

For example, referring to Table 13, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 571, optionally SEQ ID NO: 566, and SEQ ID NO: 572.

For example, referring to Table 14, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 571, optionally SEQ ID NO: 566, and SEQ ID NO: 572.

Every row of the above tables are disclosed in the same manner. Such antibodies are human IgG1 antibodies. Therefore, the human IgG1 antibodies may comprise a CH3 domain comprising the sequence of SEQ ID NO: 570.

In this aspect, the multispecific antibody may comprise SEQ ID NO: 609; or SEQ ID NO: 414 and SEQ ID NO: 607. In this aspect, the multispecific antibody may comprise SEQ ID NO: 610; or SEQ ID NO: 414 and SEQ ID NO: 608.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 570.

### ADT1-4-1×ADT5-3n

For example, referring to Table 13, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 571, optionally SEQ ID NO: 566, and SEQ ID NO: 572.

For example, referring to Table 14, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 571, optionally SEQ ID NO: 566, and SEQ ID NO: 572.

Every row of the above table is disclosed in the same manner. Such antibodies are human IgG1 antibodies. Therefore, the human IgG1 antibodies may comprise a CH3 domain comprising the sequence of SEQ ID NO: 570.

In this aspect, the multispecific antibody may comprise SEQ ID NO: 613; or SEQ ID NO: 685 and SEQ ID NO: 611. In this aspect, the multispecific antibody may comprise SEQ ID NO: 614; or SEQ ID NO: 685 and SEQ ID NO: 612.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 570.

### ADT5-4n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 573 | PD-L1 ADT5-4 CH3 binding module |
| 565 | PD-L1 ADT5-4 AB substitutions |
| 566 | PD-L1 ADT5-4 CD substitutions |
| 572 | PD-L1 ADT5-4 EF substitutions |
| 615 | PD-L1 ADT5-4n Binding Module CH1-CH2-CH3 |
| | (LAGA) |
| 616 | PD-L1 ADT5-4n Binding Module CH1-CH2-CH3 |
| | (wt) |
| 617 | ADT1-4-2HC PD-L1 ADT5-4n LAGA |
| 618 | ADT1-4-2HC PD-L1 ADT5-4n WT |
| 619 | ADT1-4-2 × ADT5-4n (LAGA) |
| 620 | ADT1-4-2 × ADT5-4n (WT) |
| 621 | ADT1-4-1HC PD-L1 ADT5-4n LAGA |
| 622 | ADT1-4-1HC PD-L1 ADT5-4n WT |
| 623 | ADT1-4-1 × ADT5-4n (LAGA) |
| 624 | ADT1-4-1 × ADT5-4n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 565, the second sequence has the sequence set forth in SEQ ID NO: 566, and the third sequence has the sequence set forth in SEQ ID NO: 572.

In this aspect, the IgG1 CH3 domain may comprise the following mutations (EU numbering). In the AB loop the following mutations may be present: L358˜.K360G.N361Y.Q362W (˜ denotes an amino acid deletion).

In the CD loop the following mutations may be present: Q386E.E388Q.N389Y.N390W.Y391A. In the EF loop the following mutations may be present: D413S.K414N.S415W. Q419L.G420D.N421 D.

In this aspect the amino acid sequence of the CH3 domain may be at least 90% identical to, or at least 95% identical to, or 100% identical to SEQ ID NO: 573.

In some embodiments, the multispecific antibodies of the disclosure may have one or both of the two residues at the immediate C-terminus of the CH3 domain sequence removed (G446 and/or K447, EU numbering).

TABLE 15
ADT5-4n - Example multispecific vδ1-PD-L1 human IgG1 antibodies having the
CDRs of antibodies derived from ADT1-4 and ADT1-7 and the related SEQ ID NOs

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 572 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 565 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 16
ADT5-4n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 572 |
| ADT1-4-112 | 5 | 30 | SEQ ID | | |
| ADT1-4-117 | 6 | 31 | NO: 565 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-4n

For example, referring to Table 15, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 565, optionally SEQ ID NO: 566, and SEQ ID NO: 572.

For example, referring to Table 16, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 565, optionally SEQ ID NO: 566, and SEQ ID NO: 572.

Every row of the above tables is disclosed in the same manner. Such antibodies are human IgG1 antibodies. Therefore, the human IgG1 antibodies may comprise a CH3 domain comprising the sequence of SEQ ID NO: 573.

In this aspect, the multispecific antibody may comprise SEQ ID NO: 619; or SEQ ID NO: 414 and SEQ ID NO: 617. In this aspect, the multispecific antibody may comprise SEQ ID NO: 620; or SEQ ID NO: 414 and SEQ ID NO: 618.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 573.

### ADT1-4-1×ADT5-4n

For example, referring to Table 15, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 565, optionally SEQ ID NO: 566, and SEQ ID NO: 572.

For example, referring to Table 16, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 565, optionally SEQ ID NO: 566, and SEQ ID NO: 572.

Every row of the above table is disclosed in the same manner. Such antibodies are human IgG1 antibodies. Therefore, the human IgG1 antibodies may comprise a CH3 domain comprising the sequence of SEQ ID NO: 573.

In this aspect, the multispecific antibody may comprise SEQ ID NO: 623; or SEQ ID NO: 685 and SEQ ID NO: 621. In this aspect, the multispecific antibody may comprise SEQ ID NO: 624; or SEQ ID NO: 685 and SEQ ID NO: 622.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 573.

### ADT5-5n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 574 | PD-L1 ADT5-5 CH3 binding module |
| 565 | PD-L1 ADT5-5 AB substitutions |
| 566 | PD-L1 ADT5-5 CD substitutions |
| 575 | PD-L1 ADT5-5 EF substitutions |
| 625 | PD-L1 ADT5-5n Binding Module CH1-CH2-CH3 |
| | (LAGA) |
| 626 | PD-L1 ADT5-5n Binding Module CH1-CH2-CH3 |
| | (wt) |
| 627 | ADT1-4-2HC PD-L1 ADT5-5n LAGA |
| 628 | ADT1-4-2HC PD-L1 ADT5-5n WT |
| 629 | ADT1-4-2 × ADT5-5n (LAGA) |
| 630 | ADT1-4-2 × ADT5-5 ADT5-5n (WT) |
| 631 | ADT1-4-1HC PD-L1 ADT5-5n LAGA |
| 632 | ADT1-4-1HC PD-L1n WT |
| 633 | ADT1-4-1 × ADT5-5n (LAGA) |
| 634 | ADT1-4-1 × ADT5-5n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 565, the second sequence has the sequence set forth in SEQ ID NO: 566, and the third sequence has the sequence set forth in SEQ ID NO: 575.

In this aspect, the IgG1 CH3 domain may comprise the following mutations (EU numbering). In the AB loop the following mutations may be present: L358˜.K360G.N361Y.Q362W (˜ denotes an amino acid deletion). In the CD loop the following mutations may be present: Q386E.E388Q.N389Y.N390W.Y391A. In the EF loop the following mutations may be present: D413S.S415W.Q419L.G420D.N421 D.

In this aspect the amino acid sequence of the CH3 domain may be at least 90% identical to, or at least 95% identical to, or 100% identical to SEQ ID NO: 574.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 625 or SEQ ID NO: 626.

In this aspect the Fab region may be any of the Fab regions described herein (see Tables 17 and 18, for example).

TABLE 17
ADT5-5n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 575 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 565 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 18
ADT5-5n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 575 |
| ADT1-4-112 | 5 | 30 | SEQ ID | | |
| ADT1-4-117 | 6 | 31 | NO: 565 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-5n

For example, referring to Table 17, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 565, optionally SEQ ID NO: 566, and SEQ ID NO: 575.

For example, referring to Table 18, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 565, optionally SEQ ID NO: 566, and SEQ ID NO: 575.

Every row of the above tables is disclosed in the same manner. Such antibodies are human IgG1 antibodies. Therefore, the human IgG1 antibodies may comprise a CH3 domain comprising the sequence of SEQ ID NO: 574.

In this aspect, the multispecific antibody may comprise SEQ ID NO: 629; or SEQ ID NO: 414 and SEQ ID NO: 627. In this aspect, the multispecific antibody may comprise SEQ ID NO: 630; or SEQ ID NO: 414 and SEQ ID NO:628.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 574.

### ADT1-4-1×ADT5-5n

For example, referring to Table 17, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 565, optionally SEQ ID NO: 566, and SEQ ID NO: 575.

For example, referring to Table 18, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 565, optionally SEQ ID NO: 566, and SEQ ID NO: 575.

Every row of the above table is disclosed in the same manner. Such antibodies are human IgG1 antibodies. Therefore, the human IgG1 antibodies may comprise a CH3 domain comprising the sequence of SEQ ID NO: 574.

In this aspect, the multispecific antibody may comprise SEQ ID NO: 633; or SEQ ID NO: 685 and SEQ ID NO: 631. In this aspect, the multispecific antibody may comprise SEQ ID NO: 634; or SEQ ID NO: 685 and SEQ ID NO: 632.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 574.

### ADT5-6n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 576 | PD-L1 ADT5-6 CH3 binding module |
| 569 | PD-L1 ADT5-6 AB substitutions |
| 566 | PD-L1 ADT5-6 CD substitutions |
| 572 | PD-L1 ADT5-6 EF substitutions |
| 635 | PD-L1 ADT5-6n Binding Module CH1-CH2-CH3 |
| | (LAGA) |
| 636 | PD-L1 ADT5-6n Binding Module CH1-CH2-CH3 |
| | (wt) |
| 637 | ADT1-4-2HC PD-L1 ADT5-6n LAGA |
| 638 | ADT1-4-2HC PD-L1 ADT5-6n WT |
| 639 | ADT1-4-2 × ADT5-6 (LAGA) |
| 640 | ADT1-4-2 × ADT5-6n (WT) |
| 641 | ADT1-4-1HC PD-L1 ADT5-6n LAGA |
| 642 | ADT1-4-1HC PD-L1 ADT5-6n WT |
| 643 | ADT1-4-1 × ADT5-6n (LAGA) |
| 644 | ADT1-4-1 × ADT5-6n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 569, the second sequence has the sequence set forth in SEQ ID NO: 566, and the third sequence has the sequence set forth in SEQ ID NO: 572.

In this aspect, the IgG1 CH3 domain may comprise the following mutations (EU numbering). In the AB loop the following mutations may be present: L358˜.T359E.K360G.N361Y.Q362W (˜ denotes an amino acid deletion). In the CD loop the following mutations may be present: Q386E.E388Q.N389Y.N390W.Y391A.

In the EF loop the following mutations may be present: D413S.K414N.S415W.Q419L.G420D.N421 D.

In this aspect the amino acid sequence of the CH3 domain may be at least 90% identical to, or at least 95% identical to, or 100% identical to SEQ ID NO: 576.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 635 or SEQ ID NO: 636.

In this aspect the Fab region may be any of the Fab regions described herein (see Tables 19 and 20, for example).

TABLE 19
ADT5-6n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 572 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 569 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 20
ADT5-6n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 572 |
| ADT1-4-112 | 5 | 30 | SEQ ID | | |
| ADT1-4-117 | 6 | 31 | NO: 569 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-6n

For example, referring to Table 19, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 569, optionally SEQ ID NO: 566, and SEQ ID NO: 572.

For example, referring to Table 20, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 569, optionally SEQ ID NO: 566, and SEQ ID NO: 572.

Every row of the above tables is disclosed in the same manner. Such antibodies are human IgG1 antibodies. Therefore, the human IgG1 antibodies may comprise a CH3 domain comprising the sequence of SEQ ID NO: 576.

In this aspect, the multispecific antibody may comprise SEQ ID NO: 639; or SEQ ID NO: 414 and SEQ ID NO: 637. In this aspect, the multispecific antibody may comprise SEQ ID NO: 640; or SEQ ID NO: 414 and SEQ ID NO: 638.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 576.

### ADT1-4-1×ADT5-6n

For example, referring to Table 19, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 569, optionally SEQ ID NO: 566, and SEQ ID NO: 572.

For example, referring to Table 20, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 569, optionally SEQ ID NO: 566, and SEQ ID NO: 572.

Every row of the above table is disclosed in the same manner. Such antibodies are human IgG1 antibodies. Therefore, the human IgG1 antibodies may comprise a CH3 domain comprising the sequence of SEQ ID NO: 576.

In this aspect, the multispecific antibody may comprise SEQ ID NO: 643; or SEQ ID NO: 685 and SEQ ID NO: 641. In this aspect, the multispecific antibody may comprise SEQ ID NO: 644; or SEQ ID NO: 685 and SEQ ID NO: 642.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 576.

### ADT5-7n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 577 | PD-L1 ADT5-7 CH3 binding module |
| 578 | PD-L1 ADT5-7 AB substitutions |
| 566 | PD-L1 ADT5-7 CD substitutions |
| 572 | PD-L1 ADT5-7 EF substitutions |
| 645 | PD-L1 ADT5-7n Binding Module CH1-CH2-CH3 |
| | (LAGA) |
| 646 | PD-L1 ADT5-7n Binding Module CH1-CH2-CH3 |
| | (wt) |
| 647 | ADT1-4-2HC PD-L1 ADT5-7n LAGA |
| 648 | ADT1-4-2HC PD-L1 ADT5-7n WT |
| 649 | ADT1-4-2 × ADT5-7n (LAGA) |
| 650 | ADT1-4-2 × ADT5-7n (WT) |
| 651 | ADT1-4-1HC PD-L1 ADT5-7n LAGA |
| 652 | ADT1-4-1HC PD-L1 ADT5-7n WT |
| 653 | ADT1-4-1 × ADT5-7n (LAGA) |
| 654 | ADT1-4-1 × ADT5-7n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 578, the second sequence has the sequence set forth in SEQ ID NO: 566, and the third sequence has the sequence set forth in SEQ ID NO: 572.

In this aspect, the IgG1 CH3 domain may comprise the following mutations (EU numbering). In the AB loop the following mutations may be present: L358˜.T359H.K360G.N361Y.Q362W (˜ denotes an amino acid deletion). In the CD loop the following mutations may be present: Q386E.E388Q.N389Y.N390W.Y391A. In the EF loop the following mutations may be present: D413S.K414N.S415W.Q419L.G420D.N421 D.

In this aspect the amino acid sequence of the CH3 domain may be at least 90% identical to, or at least 95% identical to, or 100% identical to SEQ ID NO: 577.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 645 or SEQ ID NO: 646.

In this aspect the Fab region may be any of the Fab regions described herein (see Tables 21 and 22, for example).

TABLE 21
ADT5-7n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 572 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 578 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 22
ADT5-7n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 572 |
| ADT1-4-112 | 5 | 30 | SEQ ID | | |
| ADT1-4-117 | 6 | 31 | NO: 578 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-7n

For example, referring to Table 21, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 578, optionally SEQ ID NO: 566, and SEQ ID NO: 572.

For example, referring to Table 22, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 578, optionally SEQ ID NO: 566, and SEQ ID NO: 572.

Every row of the above tables is disclosed in the same manner. Such antibodies are human IgG1 antibodies. Therefore, the human IgG1 antibodies may comprise a CH3 domain comprising the sequence of SEQ ID NO: 577.

In this aspect, the multispecific antibody may comprise SEQ ID NO: 649; or SEQ ID NO: 414 and SEQ ID NO: 647. In this aspect, the multispecific antibody may comprise SEQ ID NO: 650; or SEQ ID NO: 414 and SEQ ID NO: 648.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 577.

### ADT1-4-1×ADT5-7n

For example, referring to Table 21, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 578, optionally SEQ ID NO: 566, and SEQ ID NO: 572.

For example, referring to Table 22, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 578, optionally SEQ ID NO: 566, and SEQ ID NO: 572.

Every row of the above table is disclosed in the same manner. Such antibodies are human IgG1 antibodies. Therefore, the human IgG1 antibodies may comprise a CH3 domain comprising the sequence of SEQ ID NO: 577.

In this aspect, the multispecific antibody may comprise SEQ ID NO: 653; or SEQ ID NO: 685 and SEQ ID NO: 651. In this aspect, the multispecific antibody may comprise SEQ ID NO: 654; or SEQ ID NO: 685 and SEQ ID NO: 652.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 577.

### ADT5-8n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 579 | PD-L1 ADT5-8 CH3 binding module |
| 571 | PD-L1 ADT5-8 AB substitutions |
| 566 | PD-L1 ADT5-8 CD substitutions |
| 580 | PD-L1 ADT5-8 EF substitutions |
| 655 | PD-L1 ADT5-8n Binding Module CH1-CH2-CH3 |
| | (LAGA) |
| 656 | PD-L1 ADT5-8n Binding Module CH1-CH2-CH3 |
| | (wt) |
| 657 | ADT1-4-2HC PD-L1 ADT5-8n LAGA |
| 658 | ADT1-4-2HC PD-L1 ADT5-8n WT |
| 659 | ADT1-4-2 × ADT5-8n (LAGA) |
| 660 | ADT1-4-2 × ADT5-8n (WT) |
| 661 | ADT1-4-1HC PD-L1 ADT5-8n LAGA |
| 662 | ADT1-4-1HC PD-L1 ADT5-8n WT |
| 663 | ADT1-4-1 × ADT5-8n (LAGA) |
| 664 | ADT1-4-1 × ADT5-8n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 571, the second sequence has the sequence set forth in SEQ ID NO: 566, and the third sequence has the sequence set forth in SEQ ID NO: 580.

In this aspect, the IgG1 CH3 domain may comprise the following mutations (EU numbering). In the AB loop the following mutations may be present: L358˜.T359S.K360G.N361Y.Q362W (˜ denotes an amino acid deletion). In the CD loop the following mutations may be present: Q386E.E388Q.N389Y.N390W.Y391A. In the EF loop the following mutations may be present: D413S.K414H.S415W.Q419L.G420D.N421 D.

In this aspect the amino acid sequence of the CH3 domain may be at least 90% identical to, or at least 95% identical to, or 100% identical to SEQ ID NO: 579.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 655 or SEQ ID NO: 656.

In this aspect the Fab region may be any of the Fab regions described herein (see Tables 23 and 24, for example).

TABLE 23
ADT5-8n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 580 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 571 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 24
ADT5-8n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 580 |
| ADT1-4-112 | 5 | 30 | SEQ ID | | |
| ADT1-4-117 | 6 | 31 | NO: 571 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-8n

For example, referring to Table 23, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 571, optionally SEQ ID NO: 566, and SEQ ID NO: 580.

For example, referring to Table 24, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 571, optionally SEQ ID NO: 566, and SEQ ID NO: 580.

Every row of the above tables is disclosed in the same manner. Such antibodies are human IgG1 antibodies. Therefore, the human IgG1 antibodies may comprise a CH3 domain comprising the sequence of SEQ ID NO: 579.

In this aspect, the multispecific antibody may comprise SEQ ID NO: 659; or SEQ ID NO: 414 and SEQ ID NO: 657. In this aspect, the multispecific antibody may comprise SEQ ID NO: 660; or SEQ ID NO: 414 and SEQ ID NO:658.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 579.

### ADT1-4-1×ADT5-8n

For example, referring to Table 23, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 571, optionally SEQ ID NO: 566, and SEQ ID NO: 580.

For example, referring to Table 24, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 571, optionally SEQ ID NO: 566, and SEQ ID NO: 580.

Every row of the above table is disclosed in the same manner. Such antibodies are human IgG1 antibodies. Therefore, the human IgG1 antibodies may comprise a CH3 domain comprising the sequence of SEQ ID NO: 579.

In this aspect, the multispecific antibody may comprise SEQ ID NO: 663; or SEQ ID NO: 685 and SEQ ID NO: 661. In this aspect, the multispecific antibody may comprise SEQ ID NO: 664; or SEQ ID NO: 685 and SEQ ID NO: 662.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 579.

### ADT5-9n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 581 | PD-L1 ADT5-9 CH3 binding module |
| 571 | PD-L1 ADT5-9 AB substitutions |
| 566 | PD-L1 ADT5-9 CD substitutions |
| 582 | PD-L1 ADT5-9 EF substitutions |
| 665 | PD-L1 ADT5-9n Binding Module CH1-CH2-CH3 |
| | (LAGA) |
| 666 | PD-L1 ADT5-9n Binding Module CH1-CH2-CH3 |
| | (wt) |
| 667 | ADT1-4-2HC PD-L1 ADT5-9n LAGA |
| 668 | ADT1-4-2HC PD-L1 ADT5-9n WT |
| 669 | ADT1-4-2 × ADT5-9n (LAGA) |
| 670 | ADT1-4-2 × ADT5-9n (WT) |
| 671 | ADT1-4-1HC PD-L1 ADT5-9n LAGA |
| 672 | ADT1-4-1HC PD-L1 ADT5-9n WT |
| 673 | ADT1-4-1 × ADT5-9n (LAGA) |
| 674 | ADT1-4-1 × ADT5-9n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 571, the second sequence has the sequence set forth in SEQ ID NO: 566, and the third sequence has the sequence set forth in SEQ ID NO: 582.

In this aspect, the IgG1 CH3 domain may comprise the following mutations (EU numbering). In the AB loop the following mutations may be present: L358˜.T359S.K360G.N361Y.Q362W (˜ denotes an amino acid deletion). In the CD loop the following mutations may be present: Q386E.E388Q.N389Y.N390W.Y391A. In the EF loop the following mutations may be present: D413S. K414N. S415W. Q418R. Q419L. G420D. N421D.

In this aspect the amino acid sequence of the CH3 domain may be at least 90% identical to, or at least 95% identical to, or 100% identical to SEQ ID NO: 581.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 665 or SEQ ID NO: 666.

In this aspect the Fab region may be any of the Fab regions described herein (see Tables 25 and 26, for example).

TABLE 25
ADT5-9n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 582 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 571 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 145 | 157 | | | |

TABLE 26
ADT5-9n - Example multispecific vδ1-PD-L1 human IgG1
antibodies having the variable regions of antibodies
derived from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 582 |
| ADT1-4-112 | 5 | 30 | SEQ ID | | |
| ADT1-4-117 | 6 | 31 | NO: 571 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-9n

For example, referring to Table 25, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 571, optionally SEQ ID NO: 566, and SEQ ID NO: 582.

For example, referring to Table 26, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 571, optionally SEQ ID NO: 566, and SEQ ID NO: 582.

Every row of the above tables is disclosed in the same manner. Such antibodies are human IgG1 antibodies. Therefore, the human IgG1 antibodies may comprise a CH3 domain comprising the sequence of SEQ ID NO: 581.

In this aspect, the multispecific antibody may comprise SEQ ID NO: 669; or SEQ ID NO: 414 and SEQ ID NO: 667. In this aspect, the multispecific antibody may comprise SEQ ID NO: 670; or SEQ ID NO: 414 and SEQ ID NO: 668.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 581.

### ADT1-4-1×ADT5-9n

For example, referring to Table 25, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 571, optionally SEQ ID NO: 566, and SEQ ID NO: 582.

For example, referring to Table 26, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 571, optionally SEQ ID NO: 566, and SEQ ID NO: 582.

Every row of the above table is disclosed in the same manner. Such antibodies are human IgG1 antibodies. Therefore, the human IgG1 antibodies may comprise a CH3 domain comprising the sequence of SEQ ID NO: 581.

In this aspect, the multispecific antibody may comprise SEQ ID NO: 673; or SEQ ID NO: 685 and SEQ ID NO: 671. In this aspect, the multispecific antibody may comprise SEQ ID NO: 674; or SEQ ID NO: 685 and SEQ ID NO: 672.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 581.

### ADT5-10n—Vδ1-PD-L1 Multispecific Antibodies

In this aspect of the disclosure, a multispecific antibody is provided comprising a Fab region and an Fc region, wherein the Fab region comprises a binding site specific for the variable delta 1 (Vδ1) chain of a γδ T cell receptor (TCR); and the Fc region comprises a binding site specific for PD-L1. The Fab region specifically comprises a VH and cognate VL domain. The Fc region comprises a binding domain within a heavy chain constant domain (CH1-CH2-CH3), specifically the PD-L1 binding site is located in a CH3 domain.

| SEQ ID NO: | Description |
|---|---|
| 583 | PD-L1 ADT5-10 CH3 binding module |
| 571 | PD-L1 ADT5-10 AB substitutions |
| 566 | PD-L1 ADT5-10 CD substitutions |
| 584 | PD-L1 ADT5-10 EF substitutions |
| 675 | PD-L1 ADT5-10n Binding Module CH1-CH2-CH3 |
| | (LAGA) |
| 676 | PD-L1 ADT5-10n Binding Module CH1-CH2-CH3 |
| | (wt) |
| 677 | ADT1-4-2 HC PD-L1 ADT5-10n LAGA |
| 678 | ADT1-4-2HC PD-L1 ADT5-10n WT |
| 679 | ADT1-4-2 × ADT5-10n (LAGA) |
| 680 | ADT1-4-2 × ADT5-10n (WT) |
| 681 | ADT1-4-1HC PD-L1 ADT5-10n LAGA |
| 682 | ADT1-4-1HC PD-L1 ADT5-10n WT |
| 683 | ADT1-4-1 × ADT5-10n (LAGA) |
| 684 | ADT1-4-1 × ADT5-10n (WT) |
| 414 | ADT1-4-2 LC |
| 685 | ADT1-4-1 LC |

In this aspect, the PD-L1 binding site may be provided by an IgG1 CH3 domain which comprises an AB structural loop (residues 355 to 362 of the CH3 domain), a CD structural loop (residues 383 to 391 of the CH3 domain) and an EF structural loop (residues 413 to 422 of the CH3 domain). EU numbering is used.

In this aspect, the PD-L1 binding site may comprise a first sequence located in the AB structural loop at positions 359 to 362 (EU numbering) of the CH3 domain, wherein the PD-L1 binding site comprises an amino acid deletion at residue 358; optionally a second sequence located in the CD structural loop at positions 386 to 391 (EU numbering); and a third sequence located in the EF structural loop at positions 413 to 421 (EU numbering).

In this aspect, the PD-L1 binding site may be provided by an IgG1 CH3 domain in which the first sequence has the sequence set forth in SEQ ID NO: 571, the second sequence has the sequence set forth in SEQ ID NO: 566, and the third sequence has the sequence set forth in SEQ ID NO: 584.

In this aspect, the IgG1 CH3 domain may comprise the following mutations (EU numbering). In the AB loop the following mutations may be present: L358˜.T359S.K360G.N361Y.Q362W (˜ denotes an amino acid deletion). In the CD loop the following mutations may be present: Q386E.E388Q.N389Y.N390W.Y391A.

In the EF loop the following mutations may be present: D413S.K414N.S415W.Q418S.Q419L. G420D.N421 D.

In this aspect the amino acid sequence of the CH3 domain may be at least 90% identical to, or at least 95% identical to, or 100% identical to SEQ ID NO: 583.

In some embodiments, the multispecific antibodies of the disclosure may have one or both of the two residues at the immediate C-terminus of the CH3 domain sequence removed (G446 and/or K447, EU numbering).

In this aspect, the heavy chain constant domain may comprise SEQ ID NO: 675 or SEQ ID NO: 676.

In this aspect the Fab region may be any of the Fab regions described herein (see Tables 27 and 28, for example).

TABLE 27
ADT5-10n - Example multispecific vδ1-PD-L1 human IgG1 antibodies having the
CDRs of antibodies derived from ADT1-4 and ADT1-7 and the related SEQ ID NOS

| Fab Region | | | | | | | Fc Region (EU numbering) | | |
|---|---|---|---|---|---|---|---|---|---|
| Derived | | | | | | | | CD loop | |
| from | VH | | | VL | | | AB loop | optionally | EF loop |
| Antibody | CDR1 | CDR2 | CDR3 | CDR1 | CDR2 | CDR3 | comprises | comprises | comprises |
| Parental | 51 | 53 | 54 | 79 | 80 | 81 | Residue | Residues | Residues |
| ADT1-4 | | | | | | | 358 is | 386 to | 413 to |
| ADT1-4-105 | 51 | 53 | 55 | 79 | 80 | 82 | deleted. | 391 = | 421 = |
| ADT1-4-107 | 51 | 53 | 56 | 79 | 80 | 83 | Residues | SEQ ID | SEQ ID |
| ADT1-4-110 | 51 | 53 | 57 | 79 | 80 | 84 | 359 to | NO: 566 | NO: 584 |
| ADT1-4-112 | 51 | 53 | 58 | 79 | 80 | 85 | 362 = | | |
| ADT1-4-117 | 51 | 53 | 59 | 79 | 80 | 86 | SEQ ID | | |
| ADT1-4-19 | 51 | 53 | 60 | 79 | 80 | 87 | NO: 571 | | |
| ADT1-4-21 | 52 | 53 | 61 | 79 | 80 | 88 | | | |
| ADT1-4-31 | 51 | 53 | 62 | 79 | 80 | 89 | | | |
| ADT1-4-139 | 51 | 53 | 63 | 79 | 80 | 90 | | | |
| ADT1-4-4 | 51 | 53 | 64 | 79 | 80 | 91 | | | |
| ADT1-4-143 | 51 | 53 | 65 | 79 | 80 | 92 | | | |
| ADT1-4-53 | 52 | 53 | 66 | 79 | 80 | 93 | | | |
| ADT1-4-173 | 51 | 53 | 67 | 79 | 80 | 94 | | | |
| ADT1-4-2 | 51 | 53 | 68 | 79 | 80 | 95 | | | |
| ADT1-4-8 | 51 | 53 | 69 | 79 | 80 | 96 | | | |
| ADT1-4-82 | 51 | 53 | 70 | 79 | 80 | 97 | | | |
| ADT1-4-83 | 51 | 53 | 71 | 79 | 80 | 98 | | | |
| ADT1-4-3 | 51 | 53 | 72 | 79 | 80 | 99 | | | |
| ADT1-4-84 | 51 | 53 | 73 | 79 | 80 | 100 | | | |
| ADT1-4-86 | 52 | 53 | 74 | 79 | 80 | 101 | | | |
| ADT1-4-95 | 51 | 53 | 75 | 79 | 80 | 102 | | | |
| ADT1-4-1 | 51 | 53 | 76 | 79 | 80 | 103 | | | |
| ADT1-4-6 | 51 | 53 | 77 | 79 | 80 | 104 | | | |
| ADT1-4-138 | 51 | 53 | 78 | 79 | 80 | 105 | | | |
| Parental | 130 | 131 | 132 | 144 | 145 | 146 | | | |
| ADT1-7 | | | | | | | | | |
| ADT1-7-10 | 130 | 131 | 133 | 144 | 145 | 147 | | | |
| ADT1-7-15 | 130 | 131 | 134 | 144 | 145 | 148 | | | |
| ADT1-7-17 | 130 | 131 | 135 | 144 | 145 | 149 | | | |
| ADT1-7-18 | 130 | 131 | 136 | 144 | 145 | 150 | | | |
| ADT1-7-19 | 130 | 131 | 137 | 144 | 145 | 151 | | | |
| ADT1-7-20 | 130 | 131 | 138 | 144 | 145 | 152 | | | |
| ADT1-7-22 | 130 | 131 | 139 | 144 | 145 | 153 | | | |
| ADT1-7-23 | 130 | 131 | 140 | 144 | 145 | 154 | | | |
| ADT1-7-42 | 130 | 131 | 141 | 144 | 145 | 155 | | | |
| ADT1-7-3 | 130 | 131 | 142 | 144 | 145 | 156 | | | |
| ADT1-7-61 | 130 | 131 | 143 | 144 | 45 | 157 | | | |

TABLE 28
ADT5-10n - Example multispecific vδ1-PD-L1 human IgG1
antibodies having the variable regions of antibodies derived
from ADT1-4 and ADT1-7 and the related SEQ ID NOs

| Fab Region | | | Fc Region | | |
|---|---|---|---|---|---|
| Derived from | | | AB loop | CD loop | EF loop |
| Antibody | VH | VL | comprises | comprises | comprises |
| Parental | 1 | 26 | Residue 358 | Residues | Residues |
| ADT1-4 | | | is deleted. | 386 to | 413 to |
| ADT1-4-105 | 2 | 27 | Residues | 391 = | 421 = |
| ADT1-4-107 | 3 | 28 | 359 to | SEQ ID | SEQ ID |
| ADT1-4-110 | 4 | 29 | 362 = | NO: 566 | NO: 584 |
| ADT1-4-112 | 5 | 30 | SEQ ID | | |
| ADT1-4-117 | 6 | 31 | NO: 571 | | |
| ADT1-4-19 | 7 | 32 | | | |
| ADT1-4-21 | 8 | 33 | | | |
| ADT1-4-31 | 9 | 34 | | | |
| ADT1-4-139 | 10 | 35 | | | |
| ADT1-4-4 | 11 | 36 | | | |
| ADT1-4-143 | 12 | 37 | | | |
| ADT1-4-53 | 13 | 38 | | | |
| ADT1-4-173 | 14 | 39 | | | |
| ADT1-4-2 | 15 | 40 | | | |
| ADT1-4-8 | 16 | 41 | | | |
| ADT1-4-82 | 17 | 42 | | | |
| ADT1-4-83 | 18 | 43 | | | |
| ADT1-4-3 | 19 | 44 | | | |
| ADT1-4-84 | 20 | 45 | | | |
| ADT1-4-86 | 21 | 46 | | | |
| ADT1-4-95 | 22 | 47 | | | |
| ADT1-4-1 | 23 | 48 | | | |
| ADT1-4-6 | 24 | 49 | | | |
| ADT1-4-138 | 25 | 50 | | | |
| Parental | 106 | 118 | | | |
| ADT1-7 | | | | | |
| ADT1-7-10 | 107 | 119 | | | |
| ADT1-7-15 | 108 | 120 | | | |
| ADT1-7-17 | 109 | 121 | | | |
| ADT1-7-18 | 110 | 122 | | | |
| ADT1-7-19 | 111 | 123 | | | |
| ADT1-7-20 | 112 | 124 | | | |
| ADT1-7-22 | 113 | 125 | | | |
| ADT1-7-23 | 114 | 126 | | | |
| ADT1-7-42 | 115 | 127 | | | |
| ADT1-7-3 | 116 | 128 | | | |
| ADT1-7-61 | 117 | 129 | | | |
| C08 | 273 | 282 | | | |
| B07 | 274 | 283 | | | |
| C05 | 275 | 284 | | | |
| E04 | 276 | 285 | | | |
| F07 | 277 | 286 | | | |
| G06 | 278 | 287 | | | |
| G09 | 279 | 288 | | | |
| B09 | 280 | 289 | | | |
| G10 | 281 | 290 | | | |
| E01 | 312 | 313 | | | |

The present disclosure explicitly includes an antibody having the sequences of each row of the above tables.

### ADT1-4-2×ADT5-10n

For example, referring to Table 27, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 68, a VLCDR1 comprising the sequence of SEQ ID NO: 79, a VLCDR2 comprising the sequence of SEQ ID NO: 80 and a VLCDR3 comprising the sequence of SEQ ID NO: 95; and the Fc region comprises an PD-L1 binding site, wherein the PD-L1 binding site is provided by a CH3 domain which comprises SEQ ID NO: 571, optionally SEQ ID NO: 566, and SEQ ID NO: 584.

For example, referring to Table 28, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 15 and a VL region comprising the sequence of SEQ ID NO: 40; and the Fc region comprises a PD-L1 binding site, wherein the PD-L1 binding site is provided by a CH3 domain which comprises SEQ ID NO: 571, optionally SEQ ID NO: 566, and SEQ ID NO: 584.

Every row of the above tables is disclosed in the same manner. Such antibodies are human IgG1 antibodies. Therefore, the human IgG1 antibodies may comprise a CH3 domain comprising the sequence of SEQ ID NO: 583.

In this aspect, the multispecific antibody may comprise SEQ ID NO: 679; or SEQ ID NO: 414 and SEQ ID NO: 677. In this aspect, the multispecific antibody may comprise SEQ ID NO: 670; or SEQ ID NO: 414 and SEQ ID NO: 678.

Specifically, the Fab region of this aspect may comprise a VHCDR1, a VHCDR2 and a VHCDR3 comprising the amino acid sequences of SEQ ID NO: 51, 53 and 68, respectively, and a VLCDR1, a VLCDR2 and a VLCDR3 comprising the amino acid sequences of SEQ ID NOs: 79, 80 and 95, respectively. The Fab region of this aspect may comprise a VH comprising SEQ ID NO: 15 and a VL comprising SEQ ID NO: 40.

In one embodiment of this aspect, the multispecific antibody comprises a Fab region comprising a VH comprising the amino acid sequence of SEQ ID NO: 15 and a VL sequence comprising the amino acid sequence of SEQ ID NO: 40; and an Fc region comprising a CH3 domain comprising the amino acid sequence of SEQ ID NO: 583.

### ADT1-4-1×ADT5-10n

For example, referring to Table 27, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VHCDR1 comprising the sequence of SEQ ID NO: 51, a VHCDR2 comprising the sequence of SEQ ID NO: 53, a VHCDR3 comprising the sequence of SEQ ID NO: 76, a VLCDR1 comprising the sequence of SEQ ID NO: 79, a VLCDR2 comprising the sequence of SEQ ID NO: 80 and a VLCDR3 comprising the sequence of SEQ ID NO: 103; and the Fc region comprises an PD-L1 binding site, wherein the PD-L1 binding site is provided by a CH3 domain which comprises SEQ ID NO: 571, optionally SEQ ID NO: 566, and SEQ ID NO: 584.

For example, referring to Table 28, the present disclosure includes a multispecific antibody comprising a Fab region and an Fc region wherein the Fab region comprises a binding site specific for an epitope of the variable delta 1 (Vδ1) chain of a γδ T cell receptor (TCR), wherein the Fab region comprises a VH region comprising the sequence of SEQ ID NO: 23 and a VL region comprising the sequence of SEQ ID NO: 48; and the Fc region comprises a PD-L1 binding site, wherein the PD-L1 binding site is provided by a CH3 domain which comprises SEQ ID NO: 571, optionally SEQ ID NO: 566, and SEQ ID NO: 584.

Every row of the above table is disclosed in the same manner. Such antibodies are human IgG1 antibodies. Therefore, the human IgG1 antibodies may comprise a CH3 domain comprising the sequence of SEQ ID NO: 583.

In this aspect, the multispecific antibody may comprise SEQ ID NO: 683; or SEQ ID NO: 685 and SEQ ID NO: 681. In this aspect, the multispecific antibody may comprise SEQ ID NO: 684; or SEQ ID NO: 685 and SEQ ID NO:682.

Specifically, the Fab region of this aspect may comprise a VHCDR1, a VHCDR2 and a VHCDR3 comprising the amino acid sequences of SEQ ID NO: 51, 53 and 76, respectively, and a VLCDR1, a VLCDR2 and a VLCDR3 comprising the amino acid sequences of SEQ ID NOs: 79, 80 and 103, respectively. The Fab region of this aspect may comprise a VH comprising SEQ ID NO: 23 and a VL comprising SEQ ID NO: 48.

In one embodiment of this aspect, the multispecific antibody comprises a Fab region comprising a VH comprising the amino acid sequence of SEQ ID NO: 23 and a VL sequence comprising the amino acid sequence of SEQ ID NO: 48; and an Fc region comprising a CH3 domain comprising the amino acid sequence of SEQ ID NO: 583.

Remarkably in all said examples comprising at least one (first) binding domain targeting the Vδ1 chain of a γδ TCR, and at least one (second) binding domain targeting PD-L1 enhanced functionality was observed versus the controls and component parts (see FIGS. 29A-29F herein).

Collectively, these non-limiting examples highlight the flexibility of the multispecific antibodies or antigen-binding fragments thereof as described herein. These non-limiting examples outline that multispecific antibody approach wherein antibodies of fragments thereof targeting the germline Vδ1 chain (amino acids 1-90 of e.g. SEQ ID NO: 272) may be further enhanced by combining with PD-L1 binding domains to form multispecific antibodies.

In one embodiment multispecific antibody binding domains which target Vδ1 chain of a γδ TCR (the first target) may comprise (i) one or two or more antibody binding domains each comprising a heavy chain (VH-CH1-CH2-CH3) and a cognate light chain partner (VL-CL) and/or (ii) one or two or more antibody binding domains each comprising a heavy chain variable domain (VH, or VH-CH1) and a cognate light chain variable domain partner (VL, or VL-VC) and/or (iii) one or two or more antibody binding domains each comprising a CDR-containing antibody fragment.

In one embodiment there is provided a multispecific antibody comprising at least one first antibody-derived binding domain targeting the Vδ1 chain of a γδ TCR and which is operatively linked to at least one second antibody binding domain targeting PD-L1. Optionally, said binding domains comprise at least one or more VH and cognate VL binding domain, or one or more VH-CH1-CH2-CH3 and cognate VL-CL binding domain, or one or more antibody fragment binding domains. Said multispecific antibody comprising at least one first antibody-derived binding domain targeting the Vδ1 chain of a γδ TCR is operatively linked to a second binding domain binding PD-L1 and comprising one or more of the following heavy chain modifications in accordance with EU numbering; L358˜, K360G, N361Y, Q362W, (T359E or T359), Q386E, E388Q, N389Y, N390W, Y391A, D413S, S415W, Q419L, N421D, K414N, (Q418R or Q418S), G420D, V422˜ and/or H433R. Such heavy chain modifications are found to be advantageous.

In one aspect of the disclosure multispecific antibodies of the disclosure can be used in therapeutically effective amounts to treat a disease or disorder such to ameliorate at least one sign or symptom of a disease or disorder.

### Combinatorial Therapies

The multispecific antibodies of the disclosure may be used in certain combination therapies. In some embodiments, the multispecific antibodies may be combined with a modulator of a cancer antigen or a cancer-associated antigen, for example selected from the group consisting of AFP, AKAP-4, ALK, alpha-fetoprotein, Androgen receptor, B7H3, BAGE, BCA225, BCAA, Bcr-abl, beta-Catenin, beta-HCG, beta-human chorionic gonadotropin, BORIS, BTAA, CA 125, CA 15-3, CA 195, CA 19-9, CA 242, CA 27.29, CA 72-4, CA-50, CAM 17.1, CAM43, Carbonic anhydrase IX, carcinoembryonic antigen, CD22, CD33/IL3Ra, CD68\P1, CDK4, CEA, chondroitin sulfate proteoglycan 4 (CSPG4), c-Met, CO-029, CSPG4, Cyclin B1, cyclophilin C-associated protein, CYP1B1, E2A-PRL, EGFR, EGFRvIII, ELF2M, EpCAM, EphA2, EphrinB2, Epstein Barr virus antigens EBVA, ERG (TMPRSS2ETS fusion gene), ETV6-AML, FAP, FGF-5, Fos-related antigen 1, Fucosyl GM1, G250, Ga733EpCAM, GAGE-1, GAGE-2, GD2, GD3, glioma-associated antigen, GloboH, Glycolipid F77, GM3, GP 100, GP 100 (Pmel 17), H4-RET, HER-2/neu, HER-2/Neu/ErbB-2, high-molecular-weight melanoma-associated antigen (HMW-MAA), HPV E6, HPV E7, hTERT, HTgp-175, human telomerase reverse transcriptase, Idiotype, IGF-I receptor, IGF-II, IGH-IGK, insulin growth factor (IGF)-I, intestinal carboxyl esterase, K-ras, LAGE-1a, LCK, lectin-reactive AFP, Legumain, LMP2, M344, MA-50, Mac-2 binding protein, MAD-CT-1, MAD-CT-2, MAGE, MAGE A1, MAGE A3, MAGE-1, MAGE-3, MAGE-4, MAGE-5, MAGE-6, MART-1, MART-1/MelanA, M-CSF, melanoma-associated chondroitin sulfate proteoglycan (MCSP), Mesothelin, MG7-Ag, ML-IAP, MN-CA IX, MOV18, MUC1, Mum-1, hsp70-2, MYCN, MYL-RAR, NA17, NB/70K, neuron-glial antigen 2 (NG2), neutrophil elastase, nm-23H1, NuMa, NY-BR-1, NY-CO-1, NY-ESO, NY-ESO-1, NY-ESO-1, OY-TES1, p15, p16, p180erbB3, p185erbB2, p53, p53 mutant, Page4, PAX3, PAX5, PDGFR-beta, PLAC1, Polysialic Acid, prostate-carcinoma tumor antigen-1 (PCTA-1), prostate-specific antigen, prostatic acid phosphatase (PAP), Proteinase3 (PR1), PSA, PSCA, PSMA, RAGE-1, Ras, Ras-mutant, RCAS1, RGS5, RhoC, ROR1, RU1, RU2 (AS), SART3, SDCCAG16, sLe(a), Sperm protein 17, SSX2, STn, Survivin, TA-90, TAAL6, a TAG-72, telomerase, thyroglobulin, Tie 2, TLP, Tn, TPS, TRP-1, TRP-2, TRP-2, TSP-180, Tyrosinase, VEGF, VEGFR2, VISTA, WT1, XAGE 1, 43-9F, 5T4, and 791Tgp72.

In some embodiments, the multispecific antibodies may be combined with a modulator of a immunomodulatory antigen, for example selected from the group consisting of B7-1 (CD80), B7-2 (CD86), B7-DC (CD273), B7-H1 (CD274), B7-H2 (CD275), B7-H3 (CD276), B7-H4 (VTCN1), B7-H5 (VISTA), BTLA (CD272), CD137, CD137L, CD24, CD27, CD28, CD38, CD40, CD40L (CD154), CD54, CD59, CD70, CTLA4 (CD152), CXCL9, GITR (CD357), HVEM (CD270), ICAM-1 (CD54), ICOS (CD278), LAG-3 (CD223), OX40 (CD134), OX40L (CD252), PD-1 (CD279), PD-L1 (CD274), TIGIT, CD314, CD334, CD335, CD337, and TIM-3 (CD366).

In some embodiments, the multispecific antibodies may be combined with a modulator of a CD antigen, for example selected from the group consisting of CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD13, CD14, CD15, CD16, CD16a, CD16b, CD17, CD18, CD19, CD20, CD21, CD22, CD23, CD24, CD25, CD26, CD27, CD28, CD29, CD30, CD31, CD32A, CD32B, CD33, CD34, CD35, CD36, CD37, CD38, CD39, CD40, CD41, CD42, CD42a, CD42b, CD42c, CD42d, CD43, CD44, CD45, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64a, CD65, CD65s, CD66a, CD66b, CD66c, CD66d, CD66e, CD66f, CD68, CD69, CD70, CD71, CD72, CD73, CD74, CD75, CD75s, CD77, CD79A, CD79B, CD80, CD81, CD82, CD83, CD84, CD85A, CD85B, CD85C, CD85D, CD85F, CD85G, CD85H, CD85I, CD85J, CD85K, CD85M, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121a, CD121b, CD122, CD123, CD124, CD125, CD126, CD127, CD129, CD130, CD131, CD132, CD133, CD134, CD135, CD136, CD137, CD138, CD139, CD140A, CD140B, CD141, CD142, CD143, CD144, CDw145, CD146, CD147, CD148, CD150, CD151, CD152, CD153, CD154, CD155, CD156, CD156a, CD156b, CD156c, CD157, CD158, CD158A, CD158B1, CD158B2, CD158C, CD158D, CD158E1, CD158E2, CD158F1, CD158F2, CD158G, CD158H, CD158I, CD158J, CD158K, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD187, CD188, CD189, CD190, CD191, CD192, CD193, CD194, CD195, CD196, CD197, CDw198, CDw199, CD200, CD201, CD202b, CD203c, CD204, CD205, CD206, CD207, CD208, CD209, CD210, CDw210a, CDw210b, CD211, CD212, CD213a1, CD213a2, CD214, CD215, CD216, CD217, CD218a, CD218b, CD219, CD220, CD221, CD222, CD223, CD224, CD225, CD226, CD227, CD228, CD229, CD230, CD231, CD232, CD233, CD234, CD235a, CD235b, CD236, CD237, CD238, CD239, CD240CE, CD240D, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CDw293, CD294, CD295, CD296, CD297, CD298, CD299, CD300A, CD300C, CD301, CD302, CD303, CD304, CD305, CD306, CD307, CD307a, CD307b, CD307c, CD307d, CD307e, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD344, CD349, CD351, CD352, CD353, CD354, CD355, CD357, CD358, CD360, CD361, CD362, CD363, CD364, CD365, CD366, CD367, CD368, CD369, CD370, and CD371.

In some embodiments, the modulators may be antagonistic or agonistic. Suitable modulators include antibodies, fusion proteins or small molecules.

### Immunoconjugates

The multispecific antibodies of the present disclosure may be conjugated to a therapeutic moiety, such as a cytotoxin or a chemotherapeutic agent. Such conjugates may be referred to as immunoconjugates. As used herein, the term “immunoconjugate” refers to an antibody which is chemically or biologically linked to another moiety, such as a cytotoxin, a radioactive agent, a cytokine, an interferon, a target or reporter moiety, an enzyme, a toxin, a peptide or protein or a therapeutic agent. The antibody may be linked to the cytotoxin, radioactive agent, cytokine, interferon, target or reporter moiety, enzyme, toxin, peptide or therapeutic agent at any location along the molecule so long as it is able to bind its target. Examples of immunoconjugates include antibody drug conjugates and antibody-toxin fusion proteins. In one embodiment, the agent may be a second different antibody to Vδ1. In certain embodiments, the antibody may be conjugated to an agent specific for a tumor cell or a virally infected cell. The type of therapeutic moiety that may be conjugated to the anti-Vδ1 antibody and will take into account the condition to be treated and the desired therapeutic effect to be achieved. In one embodiment, the agent may be a second antibody, or antigen-binding fragment thereof, that binds to a molecule other than Vδ1.

### Cyno-Cross Reactivity

Suitably, the multispecific antibodies exhibit cross-reactivity to both human TRDV1 SEQ ID NO: 272 (including the polymorphic variant i.e. SEQ ID NO: 306) and cyno TRDV1 (SEQ ID NO: 308). Cross reactivity is clearly useful in providing antibodies that can be used in in vivo animal studies during pre-clinical evaluation.

The present inventors have surprisingly identified a framework mutation that can confer increased binding of antibodies, for example anti-Vδ1×PD-L1 antibodies and antigen-binding fragments thereof, to cynomolgus antigens. The framework mutation does not adversely affect the affinity of the antibody or antigen-binding fragment thereof to the corresponding human version of the antigen. The mutation is the mutation of the serine residue to position 74 according to the IMGT numbering system of a kappa light chain variable sequence to a residue that is not serine (for example a non-human-germline and/or non-polar residue). Non-polar amino acids may be selected from the group consisting of glycine, alanine, valine, methionine, leucine and isoleucine. Non-germline amino acids may be selected from the group consisting of arginine, cysteine, glutamine, glutamic acid, glycine, histidine, isoleucine, leucine, lysine, methionine, phenylalanine, threonine, tryptophan, tyrosine and valine. Non-polar and non-germline amino acids (i.e. amino acids that are both non-polar and non-germline) may be selected from the group consisting of glycine, valine, methionine, leucine and isoleucine). In some embodiments, the mutation is the mutation of the serine residue to position 74 according to the IMGT numbering system of a kappa light chain variable sequence to a leucine residue. The mutation is a direct substitution such the overall length of the chain does not change. Therefore, the serine a position 74 according to the IMGT numbering system is removed and directly replaced with another amino acid (such as a non-human-germline and/or non-polar amino acid, such as a leucine). The substitution may be achieved according to any suitable method known to the skilled person.

References herein to antibodies comprising a kappa light chain variable sequence comprising a residue at position 74 according to the IMGT numbering that is not serine may alternatively be defined as antibodies comprising a light chain variable sequence comprising a LFR1 region, a LCDR1 region, a LFR2 region, a LCDR2 region, a LFR3 region, a LCDR3 region and a LFR4 region, wherein the LFR3 region comprises a residue at position 74 according to the IMGT numbering that is not serine (for example a non-human-germline and/or non-polar amino acid, such as a leucine residue). Such an alternative definition is applicable to all antibodies disclosed herein comprising a kappa light chain variable sequence comprising a residue at position 74 according to the IMGT numbering that is not serine (for example a non-human-germline and/or non-polar amino acid, such as a leucine residue).

Accordingly, the present disclosure provides anti-TCR delta variable 1 (anti-Vδ1)×PD-L1 multispecific antibodies or antigen-binding fragments thereof comprising a kappa light chain variable sequence, in which the residue at position 74 according to the IMGT numbering system of the light chain variable sequence is a not serine (for example a non-human-germline and/or non-polar amino acid, such as a leucine residue). In some embodiments, the residue at position 74 according to the IMGT numbering system of the light chain variable sequence is a leucine. Suitably, the antibody may be an IgG antibody. For example, the antibody may be IgG1 antibody.

The anti-Vδ1×PD-L1 multispecific antibodies and antigen-binding fragments thereof provided herein may be provided with the substitution at position 74 (according to the IMGT numbering system) of the light chain variable sequence. For example, the anti-Vδ1×PD-L1 multispecific antibodies and antigen-binding fragments and variants thereof described herein may comprise a kappa light chain variable sequence comprising a residue other than serine at position 74 according to the IMGT numbering system (for example a non-human-germline and/or non-polar amino acid, such as a leucine residue). In some embodiments, the anti-Vδ1×PD-L1 multispecific antibodies and antigen-binding fragments and variants thereof described herein may comprise a kappa light chain variable sequence comprising a leucine residue at position 74 according to the IMGT numbering system. Embodiments comprising a mutation at position 74 according to the IMGT numbering system of a kappa light chain may be particularly relevant to antibodies derived from ADT1-4, although the mutation is equally applicable to other antibodies. For example, it is observed that light chain position 74 is not highly conserved across the different kappa/lambda light chain germlines. Further it is noted that differing light chain germlines contain differently polarising and/or charged amino acids at this position ranging from non-polar (e.g. alanine) to polar neutral (e.g. serine) to negatively or positively charged (e.g. aspartate or asparagine respectively). It is well understood that amino acid polarity/charge at any given position may impact protein structure. For example, it is well understood that a change in polarity and charge can impact hydrophobicity and tendency for an amino acid to be more surface exposed or more buried. Regardless, this remarkable finding highlights that non-conservative amino acid changes at light chain position 74 can modify affinity to a selected target by greater than 10-fold. Hence aside improving affinity as outlined herein, use of this knowledge may offer a new approach to dialling up or down affinity more broadly. For example, a method comprising changing the residue at position 74 from more polar to non-polar, or for example non-polar to more charged, may be more preferable to complex/cumbersome mutagenesis methods such as saturation mutagenesis etc when one desires to dial up or dial down affinity for any given antibody.

In a further embodiment of the disclosure there is also provided a method of mutating an antibody or antigen-binding fragment thereof, comprising providing an antibody comprising a kappa light chain having a serine at position 74 of the light chain variable sequence according to the IMGT numbering system, and mutating the serine to a different residue, for example a non-human-germline and/or non-polar amino acid, such as a leucine. In some embodiments, the antibody is an anti-TCR delta variable 1 (anti-Vδ1) antibody or antigen-binding fragment thereof. In some embodiments, the antibody is an antibody having a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 26 both before and after the mutation at position 74 of the light chain variable sequence is introduced. In some embodiments, the antibody is an antibody having a VH comprising an amino acid sequence that is 100% identical to SEQ ID NO: 1 with the exception of the mutation at position 74 and a VL comprising an amino acid sequence that is 100% identical to SEQ ID NO: 26 with the exception of the mutation at position 74.

In some embodiments, the antibody is an antibody having a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 106 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 118 both before and after the mutation at position 74 of the light chain variable sequence is introduced. In some embodiments, the antibody is an antibody having a VH comprising an amino acid sequence that is 100% identical to SEQ ID NO: 106 with the exception of the mutation at position 74 and a VL comprising an amino acid sequence that is 100% identical to SEQ ID NO: 118 with the exception of the mutation at position 74.

In some embodiments, the antibody is an antibody comprising:
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 273 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 282 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 274 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 283 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 275 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 284 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 276 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 285 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 277 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 286 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 278 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 287 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 279 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 288 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 280 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 289 both before and after the mutation is introduced;
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 281 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 290 both before and after the mutation is introduced; or
a VH comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 312 and a VL comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 313 both before and after the mutation is introduced.

In some embodiments, the antibody is an antibody comprising:
a VH comprising the amino acid sequence of SEQ ID NO: 273 and a VL comprising the amino acid sequence of SEQ ID NO: 282;
a VH comprising the amino acid sequence of SEQ ID NO: 274 and a VL comprising the amino acid sequence of SEQ ID NO: 283;
a VH comprising the amino acid sequence of SEQ ID NO: 275 and a VL comprising the amino acid sequence of SEQ ID NO: 284;
a VH comprising the amino acid sequence of SEQ ID NO: 276 and a VL comprising the amino acid sequence of SEQ ID NO: 285;
a VH comprising the amino acid sequence of SEQ ID NO: 277 and a VL comprising the amino acid sequence of SEQ ID NO: 286;
a VH comprising the amino acid sequence of SEQ ID NO: 278 and a VL comprising the amino acid sequence of SEQ ID NO: 287;
a VH comprising the amino acid sequence of SEQ ID NO: 279 and a VL comprising the amino acid sequence of SEQ ID NO: 288;
a VH comprising the amino acid sequence of SEQ ID NO: 280 and a VL comprising the amino acid sequence of SEQ ID NO: 289;
a VH comprising the amino acid sequence of SEQ ID NO: 281 and a VL comprising the amino acid sequence of SEQ ID NO: 290; or
a VH comprising the amino acid sequence of SEQ ID NO: 312 and a VL comprising the amino acid sequence of SEQ ID NO: 313.

The antibody produced by such methods has 100% identity to the specified VH and VL sequences, with the exception of the mutation at position 74.

In some embodiments, the mutation at position 74 increases the affinity of the antibody or antigen-binding fragment thereof for a homologous cynomolgus (cyno) monkey antigen. The increase of the affinity of the antibody or antigen-binding fragment thereof is relative to the affinity of the antibody or antigen-binding fragment thereof before the mutation was introduced (when measured under the same or substantially the same conditions). In some embodiments, the antibody or antigen-binding fragment thereof is an anti-TCR delta variable 1 (anti-Vδ1) antibody or antigen-binding fragment thereof that binds to a human variable delta 1 (Vδ1) chain of a γδ T cell receptor (TCR) (for example SEQ ID NO: 272 and 306) before and after the mutation is introduced, and the antibody has increased affinity for a cynomolgus monkey variable delta 1 (Vδ1) chain of a γδ T cell receptor (TCR) (for example SEQ ID NO: 308) after the mutation is introduced.

### Polynucleotide Sequences and Expression Vectors

In one aspect of the disclosure there is provided a polynucleotide encoding the multispecific antibody of the disclosure. In one embodiment, the polynucleotide comprises or consists of a sequence having at least 70%, such as at least 80%, such as at least 90%, such as at least 95%, such as at least 99% sequence identity with any one of SEQ ID NOs: 199 to 222, 224 to 247, 249 to 259 or 261 to 271. In a further embodiment the polynucleotide comprises or consists of any one of SEQ ID NOs: 199 to 222, 224 to 247, 249 to 259 or 261 to 271. In a further aspect there is provided a cDNA comprising said polynucleotide.

In one aspect of the disclosure there is provided a polynucleotide comprising or consisting of a sequence having at least 70%, such as at least 80%, such as at least 90%, such as at least 95%, such as at least 99% sequence identity with any one of the portions SEQ ID NOs: 199 to 222, 224 to 247, 249 to 259 or 261 to 271 which encodes CDR1, CDR2 and/or CDR3 of the encoded immunoglobulin chain variable domain.

In one aspect of the disclosure there is provided a polynucleotide comprising or consisting of a sequence having at least 70%, such as at least 80%, such as at least 90%, such as at least 95%, such as at least 99% sequence identity with any one of the portions of SEQ ID NOs: 199 to 222, 224 to 247, 249 to 259 or 261 to 271 which encodes FR1, FR2, FR3 and/or FR4 of the encoded immunoglobulin chain variable domain.

The present disclosure also provides expression vectors and plasmids comprising the polynucleotide sequences of the disclosure. In some embodiments, the expression vectors comprise a sequence having at least 70%, such as at least 80%, such as at least 90%, such as at least 95%, such as at least 99% sequence identity or 100% identity with any one of SEQ ID NOs: 199 to 222 or 249 to 259 (encoding variable heavy regions). In some embodiments, the expression vectors comprise a sequence having at least 70%, such as at least 80%, such as at least 90%, such as at least 95%, such as at least 99% sequence identity or 100% identity with any one of SEQ ID NOs: 224 to 247 or 261 to 271 (encoding variable light regions). Such expression vectors may be used in pairs, suitable pairing the heavy and light chain variable sequences according to the pairing of various amino acid sequences providing the multispecific antibodies of the disclosure disclosed herein. In some embodiments, the expression vectors comprise a sequence having at least 70%, such as at least 80%, such as at least 90%, such as at least 95%, such as at least 99% sequence identity or 100% identity with any one of SEQ ID NOs: 199 to 222 or 249 to 259 (encoding a variable heavy region) and further comprises a sequence having at least 70%, such as at least 80%, such as at least 90%, such as at least 95%, such as at least 99% sequence identity or 100% identity with any one of SEQ ID NOs: 224 to 247 or 261 to 271 (encoding a variable light region). Again, the sequences may be provided in specific pairs as described herein to encode the multispecific antibodies of the disclosure.

The present disclosure also provides polynucleotide sequences and expression vectors and plasmids encoding all of the antibody sequences disclosed herein, including any variant antibody sequences disclosed herein optionally comprising one or more amino acid substitutions.

The polynucleotides and expression vectors of the disclosure may also be described in reference to the amino acid sequence encoded. Therefore, in one embodiment, the polynucleotide comprises or consists of a sequence encoding the amino acid sequence of any one of SEQ ID NOs: 1 to 686.

To express the multispecific antibodies, or antigen-binding fragments thereof, polynucleotides encoding partial or full-length light and heavy chains, as described herein, are inserted into expression vectors such that the genes are operatively linked to transcriptional and translational control sequences. Therefore, in one aspect of the disclosure there is provided an expression vector comprising the polynucleotide sequence as defined herein. In one embodiment, the expression vector comprises the VH region of any one of SEQ ID NOs: 199 to 222 or 249 to 259. In another embodiment, the expression vector comprises the VL region of any one of SEQ ID NOs: 224 to 247 or 261 to 271.

It will be understood that the nucleotide sequences described herein may comprise additional sequences encoding amino acid residues to aid with translation, purification and detection, however alternative sequences may be used depending upon the expression system used. These optional sequences can be removed, modified or substituted if alternate design, translation, purification or detection strategies are adopted.

Mutations can be made to the DNA or cDNA that encode polypeptides which are silent as to the amino acid sequence of the polypeptide, but which provide preferred codons for translation in a particular host. The preferred codons for translation of a nucleic acid in, e.g. E. coli and S. cerevisiae, as well as mammalian, specifically human, are known.

Mutation of polypeptides can be achieved for example by substitutions, additions or deletions to a nucleic acid encoding the polypeptide. The substitutions, additions or deletions to a nucleic acid encoding the polypeptide can be introduced by many methods, including for example error-prone PCR, shuffling, oligonucleotide-directed mutagenesis, assembly PCR, PCR mutagenesis, in vivo mutagenesis, cassette mutagenesis, recursive ensemble mutagenesis, exponential ensemble mutagenesis, site-specific mutagenesis, gene reassembly, artificial gene synthesis, Gene Site Saturation Mutagenesis (GSSM), synthetic ligation reassembly (SLR) or a combination of these methods. The modifications, additions or deletions to a nucleic acid can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, ensemble mutagenesis, chimeric nucleic acid multimer creation, or a combination thereof.

In particular, artificial gene synthesis may be used. A gene encoding a polypeptide of the disclosure can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity.

Expression vectors include, for example, plasmids, retroviruses, cosmids, yeast artificial chromosomes (YACs) and Epstein-Barr virus (EBV) derived episomes. The polynucleotide is ligated into a vector such that transcriptional and translational control sequences within the vector serve their intended function of regulating the transcription and translation of the polynucleotide. Expression and/or control sequences can include promoters, enhancers, transcription terminators, a start codon (i.e. ATG) 5′ to the coding sequence, splicing signals for introns and stop codons. The expression vector and expression control sequences are chosen to be compatible with the expression host cell used. For example, the sequences may comprise nucleotide sequences encoding single chain variable fragments versions of the antibodies disclosure, comprising a VH region and a VL region joined by a synthetic linker (e.g. encoding SEQ ID NO: 291). It will be understood that polynucleotides or expression vectors of the disclosure may comprise the VH region, the VL region or both (optionally including the linker). Therefore, polynucleotides encoding the VH and VL regions can be inserted into separate vectors, alternatively sequences encoding both regions are inserted into the same expression vector. The polynucleotide(s) are inserted into the expression vector by standard methods (e.g. ligation of complementary restriction sites on the polynucleotide and vector, or blunt end ligation if no restriction sites are present).

A convenient vector is one that encodes a functionally complete human CH or CL immunoglobulin sequence, with appropriate restriction sites engineered so that any VH or VL sequence can be easily inserted and expressed, as described herein. The expression vector can also encode a signal peptide that facilitates secretion of the antibody (or antigen-binding fragment thereof) from a host cell. The polynucleotide may be cloned into the vector such that the signal peptide is linked in-frame to the amino terminus of the antibody. The signal peptide can be an immunoglobulin signal peptide or a heterologous signal peptide (i.e. a signal peptide from a non-immunoglobulin protein).

In one aspect of the disclosure there is provided a cell (e.g. a host cell, such as a recombinant host cell) comprising the polynucleotide or expression vector as defined herein. It will be understood that the cell may comprise a first vector encoding the light chain of the antibody or antigen-binding fragment thereof, and a second vector encoding the heavy chain of the antibody or antigen-binding fragment thereof. Alternatively, the heavy and light chains both encoded on the same expression vector introduced into the cell.

In one embodiment, the polynucleotide or expression vector encodes a membrane anchor or transmembrane domain fused to the antibody or antigen-binding fragment thereof, wherein the antibody or antigen-binding fragment thereof is presented on an extracellular surface of the cell.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors.

Mammalian cell lines available as hosts for expression are well known in the art and include many immortalized cell lines available from the American Type Culture Collection (ATCC®). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g. Hep G2), A549 cells, 3T3 cells, and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Antigen-binding fragments of antibodies such as the scFv and Fv fragments can be isolated and expressed in E. coli using methods known in the art.

The antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Antibodies (or fragments) of the disclosure can be obtained and manipulated using the techniques disclosed for example in Green and Sambrook, Molecular Cloning: A Laboratory Manual (2012) 4th Edition Cold Spring Harbour Laboratory Press.

Monoclonal antibodies can be produced using hybridoma technology, by fusing a specific antibody-producing B cell with a myeloma (B cell cancer) cell that is selected for its ability to grow in tissue culture and for an absence of antibody chain synthesis.

A monoclonal antibody directed against a determined antigen can, for example, be obtained by:
a) immortalizing lymphocytes obtained from the peripheral blood of an animal previously immunized with a determined antigen, with an immortal cell and preferably with myeloma cells, in order to form a hybridoma,
b) culturing the immortalized cells (hybridoma) formed and recovering the cells producing the antibodies having the desired specificity.

Alternatively, the use of a hybridoma cell is not required. Antibodies capable of binding to the target antigens as described herein may be isolated from a suitable antibody library via routine practice, for example, using the phage display, yeast display, ribosomal display, or mammalian display technology known in the art. Accordingly, monoclonal antibodies can be obtained, for example, by a process comprising the steps of:
a) cloning into vectors, especially into phage and more particularly filamentous bacteriophages, DNA or cDNA sequences obtained from lymphocytes especially peripheral blood lymphocytes of an animal (suitably previously immunized with determined antigens),
b) transforming prokaryotic cells with the above vectors in conditions allowing the production of the antibodies,
c) selecting the antibodies by subjecting them to antigen-affinity selection,
d) recovering the antibodies having the desired specificity.

Optionally, isolated polynucleotide encoding antibodies or antigen-binding fragment thereof as described herein and which bind to the Vδ1 chain of a γδ can also be readily manufactured to make sufficient quantities to be employed as a medicament to ameliorate the signs or symptoms of disease. When employed as a medicament in this manner, typically the polynucleotides of interest are first operatively linked to an expression vector or expression cassette designed to express said antibodies or antigen-binding fragment thereof in a subject or patient. Such expression cassettes and methods of delivery of polynucleotides or what are sometime termed ‘nucleic based’ medicaments are well known in the art. For recent review see Hollevoet and Declerck (2017) J. Transl. Med. 15(1): 131.

Also provided is a method for the production of an anti-Vδ1 antibody or antigen-binding fragment or variant thereof, comprising culturing a host cell of the disclosure in a cell culture medium under conditions to express the encoding nucleic acid sequence of the plasmid or vector inside the cell. The method may further comprise obtaining the anti-Vδ1 antibody or antigen-binding fragment or variant thereof from the cell culture supernatant. The obtained antibody may then be formulated into a pharmaceutical composition. Further, there is provided a method of producing cell that expresses an anti-Vδ1 antibody or antigen-binding fragment or variant thereof, comprising transfecting said cell with a plasmid or vector of the disclosure. Said cells can then be cultured for the production of the anti-Vδ1 antibody or antigen-binding fragment or variant thereof.

### Pharmaceutical Compositions

According to a further aspect of the disclosure, there is provided a composition comprising the multispecific antibodies as defined herein. In such embodiments, the composition may comprise the antibody, optionally in combination with other excipients. Also included are compositions comprising one or more additional active agents (e.g. active agents suitable for treating the diseases mentioned herein).

According to a further aspect of the disclosure, there is provided a pharmaceutical composition comprising the antibody or antigen-binding fragment thereof as defined herein, together with a pharmaceutically acceptable diluent or carrier. The multispecific antibodies of the disclosure can be incorporated into pharmaceutical compositions suitable for administration to a subject. Typically, the pharmaceutical composition comprises an antibody of the disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, salts, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, surfactants, excipients, preservatives or buffers, which enhance the shelf life or effectiveness of the antibody or antigen-binding fragment thereof may be combined with the multispecific antibodies of the disclosure in a pharmaceutical composition. In some embodiments, the pharmaceutical composition may comprise polysorbate 80 (PS80).

The compositions of this disclosure may be in a variety of forms. These include, for example, liquid, semi-solid and solid dosage forms, such as liquid solutions (e.g. injectable and infusible solutions), dispersions or suspensions, tablets, pills, powders, liposomes and suppositories. The preferred form depends on the intended mode of administration and therapeutic application. Typical preferred compositions are in the form of injectable or infusible solutions.

The preferred mode of administration is parenteral (e.g. intravenous, subcutaneous, intraperitoneal, intramuscular, intrathecal). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration.

The skilled person is well aware of suitable formulations for antibody medicaments and is able to select these appropriately.

It is within the scope of the disclosure to use the pharmaceutical composition of the disclosure in therapeutic methods for the treatment of diseases as described herein as an adjunct to, or in conjunction with, other established therapies normally used in the treatment of such diseases.

In a further aspect of the disclosure, the antibody, composition or pharmaceutical composition is administered sequentially, simultaneously or separately with at least one active agent.

### Treatment Methods

According to a further aspect of the disclosure, there is provided an isolated multispecific antibody as defined herein for use as a medicament. References herein to an antibody “for use” as a medicament or in therapy are limited to administration of the antibody to a subject.

In one embodiment, the multispecific antibody is for use in the treatment of cancer. In one embodiment, the disclosure is a method of treating a disease or disorder in a subject in need thereof, comprising the step of administering an multispecific antibody to the subject. In various embodiments, the disease or disorder is cancer. In one embodiment, the multispecific antibody is for use in the treatment of cancer, leads to the death of diseased cells while sparing healthy cells. In a further embodiment, the antibody or antigen-binding fragment thereof is for use in the treatment of cancer.

In one embodiment, the antibody or antigen-binding fragment thereof is for use in the treatment of cancer. In a further embodiment, the antibody or antigen-binding fragment thereof is for use in the treatment of cancer.

According to a further aspect of the disclosure, there is provided the pharmaceutical composition as defined herein for use as a medicament. In one embodiment, the pharmaceutical composition is for use in the treatment of cancer. In a further embodiment, the pharmaceutical composition is for use in the treatment of cancer.

According to a further aspect of the disclosure, there is provided a method of modulating an immune response in a subject in need thereof comprising administering a therapeutically effective amount of the isolated multispecific antibody as defined herein. In various embodiments, modulating an immune response in a subject comprises binding or targeting γδ T cells, activating γδ T cells, causing or increasing proliferation of γδ T cells, causing or increasing expansion of γδ T cells, causing or increasing γδ T cell degranulation, causing or increasing γδ T cell mediated killing activity, causing or increasing γδ T cell mediated killing activity while sparing healthy cells, causing or increasing γδ T cytotoxicity, causing or increasing γδ T cytotoxicity while sparing healthy cells, causing or increasing γδ T cell mobilization, increasing survival of γδ T cells, or increasing resistance to exhaustion of γδ T cells. Modulating the immune response in a subject may further comprise binding or targeting the second antigen. For example, in some embodiments, binding of the second antigen (PD-L1), may stimulate immunomodulation via the second antigen in addition to immunomodulation via binding of the multispecific antibody to TRDV1. Hence the modulation of the immune response may comprise modulation via two different signalling pathways, a first signalling pathway modulated via TRDV1 antigen engagement and a second signalling pathway modulated via engagement of a second immunomodulatory antigen.

According to a further aspect of the disclosure, there is provided method of treating a cancer in a subject in need thereof, comprising administering a therapeutically effective amount of the multispecific antibody as defined herein. Alternatively, a therapeutically effective amount of the pharmaceutical composition is administered.

According to further aspects of the disclosure, there is provided the use of an antibody or antigen-binding fragment thereof as defined herein for the manufacture of a medicament, for example in the treatment of cancer.

In one embodiment, the antibody or antigen-binding fragment thereof is administered to a subject, wherein the subject has cancer.

According to a further aspect of the disclosure, there is provided the pharmaceutical composition as defined herein for use as a medicament. In one embodiment, the pharmaceutical composition is administered to a subject, wherein the subject has cancer.

According to a further aspect of the disclosure, there is provided a method of administering a therapeutically effective amount of the isolated multispecific antibody as defined herein to a subject, wherein the subject has cancer. Alternatively, a therapeutically effective amount of the pharmaceutical composition is administered.

According to further aspects of the disclosure, there is provided the use of an antibody or antigen-binding fragment thereof as defined herein for the manufacture of a medicament, for example for the administration to a subject, wherein the subject has cancer.

In various embodiments, the cancer that can be treated by the disclosed methods and compositions is treated while healthy cells are spared.

In various embodiments, the cancer that can be treated by the disclosed methods and compositions include PD-L1 positive (PD-L1+) cancers. PD-L1 positive cancers (meaning cancers that comprise cancerous cells that express PD-L1 on the cell surface) include various solid malignancies such as head and neck squamous cell carcinoma (HNSCC), squamous cell carcinoma of the lung (e.g. non-small cell lung cancer (NSCLC)), gastrointestinal tract, and colorectum. The skilled person is able to ascertain whether a particular cancer is “PD-L1 positive” or “PD-L1 negative” via methods known in the art, such as immunohistochemistry (IHC) assays.

In various embodiments, the cancer that can be treated by the disclosed methods and compositions include head and neck squamous cell carcinoma (HNSCC), non-small cell lung cancer (NSCLC), colorectal cancer (CRC), and gastric cancers.

In one embodiment, the disclosure is a method of activating at least one γδ T cell in a subject, comprising the step of administering a multispecific antibody as defined herein.

In one embodiment, the disclosure is a method of causing or increasing proliferation of γδ T cells in a subject, comprising the step of administering to the subject a multispecific antibody as defined herein.

In one embodiment, the disclosure is a method of causing or increasing γδ T cell degranulation in a subject, comprising the step of administering to the subject a multispecific antibody as defined herein.

In one embodiment, the disclosure is a method of causing or increasing γδ T cell killing activity (e.g. T cell mediated killing activity) in a subject, comprising the step of administering to the subject an anti-Vδ1 antibody or antigen-binding fragment thereof as defined herein. In one embodiment, the disclosure is a method of causing or increasing γδ T cell killing activity (e.g. T cell mediated killing activity) in a subject, while sparing healthy cells, comprising the step of administering to the subject an anti-Vδ1 antibody or antigen-binding fragment thereof as defined herein.

In one embodiment, the disclosure is a method of causing or increasing γδ T cytotoxicity in a subject, comprising the step of administering to the subject an anti-Vδ1 antibody or antigen-binding fragment thereof as defined herein. In one embodiment, the disclosure is a method of causing or increasing γδ T cytotoxicity in a subject, while sparing healthy cells, comprising the step of administering to the subject an anti-Vδ1 antibody or antigen-binding fragment thereof as defined herein.

In one embodiment, the disclosure is a method of causing or increasing γδ T cell mobilization in a subject, comprising the step of administering to the subject multispecific antibody as defined herein.

In one embodiment, the disclosure is a method of increasing survival of γδ T cells in a subject, comprising the step of administering to the subject a multispecific antibody as defined herein.

In one embodiment, the disclosure is a method of or increasing resistance to exhaustion of γδ T cells in a subject, comprising the step of administering to the subject a multispecific antibody as defined herein.

According to a further aspect of the disclosure, there is provided a method of stimulating an immune response in a subject, the method comprising administration to the subject multispecific antibody in an amount effective at stimulating an immune response.

### Uses of Multispecific Antibodies or Antigen-Binding Fragments Thereof

According to a further aspect of the disclosure, there is provided the use of a multispecific antibody as described herein to engage Vδ1 γδ T cells and PDL1+ cancer cells. This may be by enabling Vδ1 immunological synapse formation.

According to a further aspect of the disclosure, there is provided the use of a multispecific antibody as described herein to mediate activation of blood and tumor resident Vδ1 γδ cells.

According to a further aspect of the disclosure, there is provided the use of a multispecific antibody as described herein to induce proliferation and activation of peripheral Vδ1 γδ T cells. Peripheral Vδ1 T cells are those outside of the tumour environment, such as blood localised Vδ1 γδ T cells in the blood.

According to a further aspect of the disclosure, there is provided the use of a multispecific antibody as described herein to trigger activation and proliferation of Vδ1 γδ cells in a tumour free system.

According to a further aspect of the disclosure, there is provided the use of a multispecific antibody as described herein to prime peripheral Vδ1 γδ T cells. Priming T cells prefers to enhancing expression of cell surface activation markers (see Example 20, FIGS. 44A-44B). This priming can take place in a tumour free environment.

According to a further aspect of the disclosure, there is provided the use of a multispecific antibody as described herein to direct blood and tumor resident Vδ1 T cells to target PD-L1+ tumor cells (tumour homing).

According to a further aspect of the disclosure, there is provided the use of a multispecific antibody as described herein to activate non-Vδ1 immune cell subsets via secretion of immune stimulatory cytokines.

This includes natural killer (NK) cells and αβ T cells.

According to a further aspect of the disclosure, there is provided the use of a multispecific antibody as described herein to study antigen recognition, activation, signal transduction or function of γδ T cells (in particular Vδ1 T cells). As described herein, the antibodies have been shown to be active in assays which can be used to investigate γδ T cell function. Such antibodies may also be useful for inducing the proliferation of γδ T cells, therefore may be used in methods of expanding γδ T cells (such as Vδ1 T cells).

Antibodies which bind to the Vδ1 chain can be used to detect γδ T cells. For example, the antibody may be labelled with a detectable label or reporter molecule or used as a capture ligand to selectively detect and/or isolate Vδ1 T cells in a sample. Labelled antibodies find use in many methods known in the art, for example immunohistochemistry and ELISA.

The detectable label or reporter molecule can be a radioisotope, such as ³H, ¹⁴C ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Fluorescent labels applied to multispecific antibodies of the disclosure may then be used in fluorescence-activated cell sorting (FACS) methods.

Thus in various embodiments, the disclosure includes in vivo methods of modulating γδ T cells, methods of binding γδ T cells, methods of targeting γδ T cells, methods of activating γδ T cells, methods of proliferating γδ T cells, methods of expanding γδ T cells, methods of detecting γδ T cells, methods of causing γδ T cell degranulation, methods of causing γδ T cell mediated killing activity, methods of selecting antibodies or antigen-binding fragments thereof, the methods comprising the step of administering a multispecific antibody or antigen-binding fragment thereof to a subject as described herein.

### Medicaments for Modulating Gamma Delta T Cells

The multispecific antibody as described herein may be used to modulate or useful for modulating delta variable 1 chain (Vδ1) T cells in a patient in situ (i.e. in vivo). The multispecific antibodies or antigen-binding fragments thereof may be comprised in medicaments for such purposes.

Modulation of Vδ1 T cells may include:
expansion of the Vδ1 T cells, e.g. by selectively increasing the number of Vδ1 T cells or promotion of survival of Vδ1 T cells;
stimulation of the Vδ1 T cells, e.g. by increasing Vδ1 T cell potency, i.e. increasing target cell killing;
prevention of Vδ1 T cell exhaustion, e.g. by increasing persistence of the Vδ1 T cells;
degranulation of Vδ1 T cells;
increase in NCR expression;
immunomodulation of the Vδ1 T cells, e.g. by downregulation of Vδ1 TCR cell surface expression, i.e. by causing Vδ1 TCR internalisation or reduced expression of Vδ1 TCR protein, or blocking the Vδ1 TCR from binding; and/or
downregulation of a TCR/CD3 complex.

Unlike anti-Vδ1 antibodies of the prior art which focus on depletion of Vδ1 T-cells, the antibodies of the present disclosure are useful for the activation of Vδ1 T-cells via the TRDV1-binding domain. Although they may cause downregulation of the TCRs on T-cells to which they bind, they do not cause Vδ1 T-cell depletion, but rather they stimulate the T-cells and hence may be useful in therapeutic settings that would benefit from the activation of this compartment of T-cells. Activation of Vδ1 T-cells is evident through TCR downregulation, CD3 downregulation, changes in activation markers such as CD25 and Ki-67 and degranulation marker CD107a. Activation of Vδ1 T-cell in turn triggers release of inflammatory cytokines such as INFγ and TNFα to promote immune licensing.

Medicaments that Modulate Immune Cell Markers on Vδ1+ Cells

The antibody or antigen-binding fragment thereof may modulate immune cell markers of Vδ1+ cells upon administration to a patient.

An antibody or antigen-binding fragment thereof described herein may also be assessed for its suitability for its therapeutic use by measuring γδ T modulation. For example, by measuring a change in the levels of CD25 or CD69 or CD107a present on a Vδ1+ T-cell or cells in a model system. Such markers are often used as markers of lymphocyte modulation (e.g. proliferation or degranulation) and can be measured following application of an antibody or antigen-binding fragment thereof as described herein, e.g. by flow cytometry. Surprisingly, during such assessments (e.g. see e.g. Examples 7, 17, 18) it was observed that antibodies as described herein conferred measurably higher levels of CD25 or CD69 or CD107a levels on target Vδ1+ T-cells. Optionally, the change in phenotype of a Vδ1+ cell or population thereof tested in the model system can then be compared to the change in phenotype when an alternative comparator antibody is applied (e.g. OKT-3, TS8.2, etc.) to said equivalent γδ T cells.

Hence in one aspect of the disclosure, there is provided a method of assessing an antibody or antigen-binding fragment thereof which binds to the Vδ1 chain of a γδ TCR for therapeutic use comprising administering the antibody or antigen-binding fragment thereof to a cell population comprising Vδ1+ cells and determining the effect on the level of CD25 and/or CD69 and/or CD107a on the surface of the Vδ1+ cells. The effect on the level of CD25, CD69 and/or CD107a may be determined/measured over a period of time. It will be understood that the effect can be measured in comparison to the level of CD25 and/or CD69 and/or CD107a on the surface of the Vδ1+ cell when said antibody is not applied to said cell over the same period of time. In a further aspect of the disclosure there is provided a method of selecting or characterizing or comparing the multispecific antibodies or antigen-binding fragments thereof as described herein which bind to the Vδ1 chain of a γδ TCR by adding said antibodies to a cell population comprising Vδ1+ cells and then measuring the level (or expression) of CD25 or CD69 or CD107a on the surface of said Vδ1+ cells.

Medicaments that Modulate Growth Properties or Numbers of Vδ1+ Cells

The antibody or antigen-binding fragment thereof may modulate the growth properties of Vδ1+ cells upon administration to a patient. For example, the antibody or antigen-binding fragment thereof may expand Vδ1+ cells.

An alternate approach to measuring γδ T proliferation may include measuring the change in relative number of Vδ1+ cells over time when applying an antibody or antigen-binding fragment thereof as described herein to model systems containing said cells. Surprisingly, during such assessments it was observed that antibodies as described herein where able to measurably increase the number of said Vδ1+ T-cells. Optionally this change in number can then be compared to the change in number observed when an alternative comparator antibody is applied (e.g. anti-OKT3) to said model systems.

Hence in another aspect of the disclosure, there is provided a method of assessing an antibody or antigen-binding fragment thereof which binds to the Vδ1 chain of a γδ TCR comprising administering the antibody or antigen-binding fragment thereof to a cell population comprising Vδ1+ cells and determining the effect on the number of Vδ1+ cells in the population. The effect on cell number can be determined/measured over a period of time. It will be understood that the effect can be measured in comparison to the effect on cell numbers observed when said antibody is not applied to the cell population for the same period of time. In a further aspect of the disclosure there is provided a method of selecting or characterizing or comparing antibodies or antigen-binding fragment thereof as described herein which bind to the Vδ1 chain of a γδ TCR by applying said antibodies to a cell population comprising Vδ1+ cells and then measuring the number of said cells over time.

Medicaments that Modulate the Proliferative Capacity and Numbers of Vδ1+ Cells

An ideal therapeutic antibody or antigen-binding fragment thereof as described herein which binds to the Vδ1 chain of a γδ TCR may be one that is capable of enhancing the proliferation of Vδ1+ cells in vivo. Such antibodies can then be employed as medicaments designed to specifically increase the Vδ1+ cell number in a subject or patient. For example:

### Cancer:

Relative increases in the numbers of Vδ1+ cells have been reported as a positive prognostic indicator associated with improved outcomes for many cancer (for example see Gentles et al (2015) Nature Immunology 21: 938-945; Wu et al. (2019) Sci. Trans. Med. 11(513): eaax9364; Catellani et al. (2007) Blood 109(5): 2078-2085). In one embodiment, presented herein is a medicament capable of increasing the relative or absolute numbers of Vδ1+ cells in situ within in a cancer patient.

### Pathogenic/Parasitic/Viral Infections:

Vδ1+ cell enrichment is observed during host defense against numerous acquired pathogenic/parasitic/viral infections. For recent general review see Zhao et al. (2018) Immunol. Res. 2018:5081634. Furthermore, increased numbers Vδ1+ are also considered protective against a variety of DNA and RNA viral infections. For example, increased numbers are also considered protective during CMV infections associated with allogeneic transplants (see van Dorp et al. (2011) Biology of Blood and Marrow Transplantation 17(2): S217). Additionally, Vδ+ cell numbers increase in patients with coronavirus infection (Poccia et al. (2006) J. Infect. Dis. 193(9): 1244-1249).

In another embodiment, presented herein is a medicament capable of increasing the relative or absolute numbers of Vδ1+ cells in a subject or patient harboring a pathogenic infection.

### Stem Cell Transplant:

Increased numbers of Vδ1+ cells have also been associated with less disease relapse, fewer viral infections, higher overall and disease-free survival and favorable clinical outcomes in general during hematopoietic stem cell transplant (for example see Aruda et al. (2019) Blood 3(21): 3436-3448 and see Godder et al. (2007) Bone Marrow Transplantation 39: 751-757). Hence another embodiment, presented herein is a medicament capable of increasing the relative or absolute numbers of Vδ1+ cells in a subject as part of a treatment regimen supporting a stem cell transplant.

Consequently, a medicament capable of preferentially or specifically increasing the numbers of Vδ1+ cells in-situ is highly desirable.

As described above, a medicament capable of preferentially or specifically increasing the numbers of peripheral Vδ1+ cells in-situ is even more desirable. According to a further aspect of the disclosure, there is provided the use of a multispecific antibody as described herein to mediate activation of blood and tumor resident Vδ1 γδ cells. According to a further aspect of the disclosure, there is provided the use of a multispecific antibody as described herein to induce proliferation and activation of peripheral Vδ1 γδ T cells. Peripheral Vδ1 T cells are those outside of the tumour environment, such as blood localised Vδ1 γδ T cells. According to a further aspect of the disclosure, there is provided the use of a multispecific antibody as described herein to trigger activation and proliferation of Vδ1 γδ cells in a tumour free system.

Medicaments that Maintain or Induce or Increase Vδ1+ Cell Cytokine Secretion

Cytokines are a large group of proteins, peptides or glycoproteins that are secreted by specific cells of immune system. They are a category of signaling molecules that mediate and regulate immunity, inflammation, and hematopoiesis. A number of cytokines have been implicated in ameliorating signs and symptoms of disease through either direct or indirect modulation of the tumour and cellular microenvironment, autoimmune tissue and associated microenvironment, or virally infected tissue or cellular environment. Exemplar pro-inflammatory cytokines include tumour necrosis factor-alpha (TNFα) and Interferon-gamma (IFNγ).

However, many such cytokines exhibit unfavourable toxicity when dosed systemically. For example, whilst TNFα can induce the haemorrhagic necrosis of transplanted tumours, and has been reported to exert synergic anti-tumour effects when combined with other chemotherapeutic drugs, various clinical trials with systemic recombinant human TNFα (rhTNFα) have highlighted significant dose limiting side effects inclusive of hypotension, rigors, phlebitis, thrombocytopenia, leukopenia and hepatotoxicity, fever, fatigue, nausea/vomiting, malaise and weakness, headache, chest tightness, low back pain, diarrhoea and shortness of breath.

Use of recombinant IFNγ also faces similar systemic toxicity challenges. For example, whilst in a cancer setting IFNγ can exert favorable pleiotropic effects including MHC class I and II upregulation to stimulate anti-tumour immunity, increasing T-cell infiltration, conferring anti-angiogenesis effects, inducing chemokine/cytokine secretion, and exerting direct cancer cell anti-proliferative effects, adverse side-effects are also observed. These include fever, headache, chills, fatigue, diarrhea, nausea, vomiting, anorexia, transient increases in hepatic transaminase, and transient decreases in granulocyte and leucocyte counts.

For a recent review on both the potential and limitation of systemic recombinant TNFα and IFNγ see Shen et al. (2018) Cell Prolif. 51(4): e12441.

Hence there is a need for more in situ controlled, more localized, more tissue or cell specific production of such cytokines. For example, more controlled expression or induction of pro-inflammatory cytokines is proposed as one approach whereby “cold” tumours can be turned “hot”. Hot tumours are also sometimes termed “T-cell-inflamed” because of an increase in the number or density of CD45+ T-cells also observed. See Bonaventura et al. (2019) Front. Immunol. 10: 168 for a recent review.

For such reasons, an ideal therapeutic antibody or antigen-binding fragment thereof as described herein which binds to the Vδ1 chain of a γδ TCR may be one that can maintain or enhance or induce the secretion of cytokines in Vδ1+ cells in vivo. Such antibodies can then be employed as medicaments designed to specifically increase or induce cytokines in a subject or patient and in a more localized, less systemic manner and one which better correlates with the distribution of Vδ1+ cells in said subject or patient.

Remarkably, when antibodies as described herein which bind to the Vδ1 chain of a γδ TCR are applied to Vδ1+ cells, a significantly higher level of secreted cytokines are observed. More specifically, and as a non-limiting example, a significant higher level of TNFα and IFNγ is observed. See e.g. Example 15.

Hence in another aspect of the disclosure, there is provided a method of assessing an antibody or antigen-binding fragment thereof which binds to the Vδ1 chain of a γδ TCR comprising administering the antibody or antigen-binding fragment thereof to a cell population comprising Vδ1+ cells and determining the amount of at least one cytokine produced by the cell population. The amount of cytokine produced can be determined/measured over a period of time and optionally compared to the amount observed when said antibody is not applied to the cell population for the same period of time. In one embodiment, the observed level of cytokine produced when the antibody is administered to the cell population is more than about 10%, more than about 20%, more than about 30%, more than about 50%, more than about 100%, more than about 150%, more than about 200%, more than about 250%, more than about 300%, more than about 350%, more than about 400%, more than about 450%, more than about 500%, more than about 1000%, relative to the level of cytokine produced when the antibody is not applied. In a further aspect of the disclosure, the cytokine is a pro-Inflammatory cytokine. In a further aspect of the disclosure, the cytokine is the TNF-α cytokine. In a further aspect of the disclosure, is IFN-γ cytokine.

In a further aspect of the disclosure there is provided a method of selecting or characterizing or comparing antibodies or antigen-binding fragment thereof as described herein which bind to the Vδ1 chain of a γδ TCR by applying said antibodies to a cell population comprising Vδ1+ cells and then measuring the level of at least one cytokine generated. In a further aspect of the disclosure the cytokine measured is TNF-α cytokine and/or IFN-γ cytokine.

In a further aspect of the disclosure, there is provided a method of assessing an antibody or antigen-binding fragment thereof which binds to the Vδ1 chain of a γδ TCR by applying said antibody or antigen-binding fragment thereof to a cell population comprising Vδ1+ cells and measuring the effect of the antibody on modulating a colder or cold tumour to become a hotter or hot tumour by determining the quantity of proinflammatory cytokines produced and/or the number or density of CD45+ T-cells present in the tumour or tumour microenvironment.

Medicaments that Expand Polyclonal Vδ1+ Cell Populations

An ideal antibody medicament may also be one designed to ensure the expanding Vδ1+ cells do not become too clonally focused at the hypervariable CDR3 sequence level. Hence an ideal antibody medicament may be designed such to avoid inducing proliferation Vδ1+ cells by binding to specific or ‘private’ δ1+CDR3 sequence paratopes. Rather, the antibody may bind via conserved germline sequences present on all Vδ1+ T cell receptors and in a gamma-chain independent manner, rather than bind to sequences presented only a sub-set of Vδ1+ cells.

Hence an ideal antibody medicament may stimulate the expansion Vδ1+ cells to generate a plurality of Vδ1+ cells containing a mixture of CDR3 sequences. This in turn would result in an in vivo expanded heterogenous polyclonal population of Vδ1+ cells displaying different CDR3 sequences on delta variable 1 chains. Remarkably, during analysis of expanded Vδ1+ cell populations generated by a method of adding an antibody or antigen-binding fragment thereof as described herein to a starting population of immune cells containing Vδ1+ cells, extensive polyclonality is observed by RNAseq based methodologies designed to sequence through the CDR3 hypervariable regions of RNA extracted (see e.g. Example 10).

Accordingly in one aspect, there is provided a method of assessing an antibody or antigen-binding fragment thereof which binds to the Vδ1 chain of a γδ TCR comprising administering the antibody or antigen-binding fragment thereof to a cell population comprising Vδ1+ cells and determining the polyclonality of the expanded Vδ1+ cells. It is desirable for an antibody medicament to generate an expanded polyclonal population containing a plurality of Vδ1+CDR3 sequences. Polyclonality can be determined using methods known in the art, such as by nucleic acid sequencing approaches capable of analysing the Vδ1 chain hypervariable CDR3 content of said Vδ1+ cells.

An ideal antibody medicament may be able to enhance or promote or stimulate the proliferation of primary Vδ1+ cells without exhausting such cells in vivo. For example, and by way of comparison, anti-CD3 medicaments such as OKT3 (e.g. Muronomab), whilst capable of expanding CD3 positive T-cells may also exhaust or induce anergy. To assess the capacity of antibodies as described herein and which bind to the Vδ1 chain of a γδ TCR to drive continued cell division of viable Vδ1+ cells, longer term proliferation studies were undertaken. Remarkably these studies revealed that antibodies as described herein and which bind to the Vδ1 chain of a γδ TCR are capable of driving cell division/proliferation of viable and still functionally cytotoxic Vδ1+ cells for over 40 days (see e.g. Example 10).

In one embodiment, there is provided a method of assessing an antibody or antigen-binding fragment thereof which binds to the Vδ1 chain of a γδ TCR comprising applying the antibody or antigen-binding fragment thereof to a cell population and monitoring the length of time Vδ1+ cell division occurs. Ideally, the antibody is capable of stimulating Vδ1+ cell division for a period of 5 to 60 days, such as at least 7 to 45 days, 7 to 21 days, or 7 to 18 days.

In a further embodiment, there provided an antibody or antigen-binding fragment thereof as described herein which binds to the Vδ1 chain of a γδ TCR and which when administered to a patient is capable of stimulating Vδ1+ cell division to increase the number by at least 2-fold in number, at least 5-fold in number, at least 10-fold in number, at least 25-fold in number, at least 50-fold in number, at least 60-fold in number, at least 70-fold in number, at least 80-fold in number, at least 90-fold in number, at least 100-fold in number, at least 200-fold in number, at least 300-fold in number, at least 400-fold in number, at least 500-fold in number, at 600-fold in number, or at least 1,000-fold in number.

In a further aspect of the disclosure there is provided a method of selecting or characterizing or comparing antibodies or antigen-binding fragment thereof as described herein which bind to the Vδ1 chain of a γδ TCR by applying said antibodies to Vδ1+ cells or mixed cell population containing Vδ1+ cells and then measuring Vδ1+ cell numbers over time.

Medicaments that Modulate Non-Vδ1+ Immune Cells Through Targeting Vδ1+ Immune Cells

An antibody or antigen-binding fragment thereof as described herein may also be assessed by measuring Vδ1+ cell mediated modulation of other immune cells. For example, a change observed in a non-γδ T cell ‘fraction’ can be measured following application of an antibody or antigen-binding fragment thereof as described herein to a model system comprising mixed population of immune cells such as one comprising human tissue αβ cells and γδ T cells. Further, the effect on non-γδ cell types in said models can be measured by flow cytometry. For example, by measuring the relative change in numbers of CD8+ αβ T cells upon addition of an antibody or antigen-binding fragment thereof as described herein to mixed cultures comprising γδ T cells and non-γδ T cells. Optionally, the observed change in number or phenotype of a non-γδ T-cell CD8+ lymphocyte population can then be compared to the change in number when an alternative comparator antibody is applied (e.g. OKT-3) to said mixed population.

Hence in another aspect of the disclosure, there is provided a method of assessing an antibody or antigen-binding fragment thereof which binds to the Vδ1 chain of a γδ TCR comprising administering the antibody or antigen-binding fragment thereof to a mixed population of immune cells or tissues comprising Vδ1+ cells and Vδ1-negative immune cells and measuring the effect on the Vδ1-negative immune cells. The effect can be determined/measured over a period of time and optionally compared to the effect observed in Vδ1-negative cells when said antibody is not applied for the same period of time. The effect may be measured as a change in the number of Vδ1-negative immune cells. For example, the antibody may increase the number Vδ1-negative immune cells by more than about 10%, more than about 20%, more than about 30%, more than about 40%, more than about 50%, more than about 70%, more than about 80%, more than about 90%, more than about 100%, more than about 500%, relative to the levels observed when said antibody is not applied.

In a further aspect of the disclosure the modulated Vδ1-negative cell is a CD45+ cell. In a further aspect of the disclosure the modulated cell is a as T-cell. In a further aspect of the disclosure the modulated αβ+ cell is CD8+ lymphocyte. In a further aspect of the disclosure the modulated as T-cell, or population thereof, exhibits evidence of enhanced cell division. In a further aspect of the disclosure there is provided a method of selecting or characterizing or comparing antibodies or antigen-binding fragment thereof as described herein which bind to the Vδ1 chain of a γδ TCR by administering said antibodies to a population of mixed immune cells comprising Vδ1+ cells and Vδ1-negative immune cells and then measuring an effect conferred on the Vδ1-negative cell population by Vδ1+ cells modulated by said multispecific antibodies or antigen-binding fragments thereof.

Optionally, and during “Vδ1+ cell mediated immune system modulation” as conferred by an antibody or antigen-binding fragment thereof as described herein, a concomitant increase in Vδ1+ cell number is also observed. And whilst not being bound by this theory, it is possible that said increase in Vδ1+ cell number may be causal in driving the concomitant expansion of co-present Vδ1-negative immune cells, such as ap T-cells. An alternate hypothesis may be that antibody-induced cytokine secretions from the Vδ1+ T cells stimulate the expansion of Vδ1-negative immune cells.

In a further aspect of the disclosure the observed increase in αβ+CD8+ lymphocyte population is compared to a comparator antibody such as OKT3 antibody or alternate anti-Vδ1 antibody. In a further aspect of the disclosure there is provided a method of selecting or characterizing or comparing antibodies or antigen-binding fragment thereof as described herein which bind to the Vδ1 chain of a γδ TCR by applying said antibodies to a population of mixed immune cells comprising Vδ1+ T-cells and as T-cells and then measuring the numbers of CD8+αβ+ T-cells lymphocytes over time.

Medicaments that Modulate Tumour Infiltrating Lymphocytes (TILs)

An antibody or antigen-binding fragment thereof as described herein may also be assessed by measuring the effect conferred on tumour-infiltrating populations (TILs) in model systems. Surprisingly (see e.g. Example 18) during such assessments antibodies as described herein measurably modulated TIL populations in human tumours. For example, a change in either the number or phenotype of γδ+ lymphocyte TIL population or the non-γδ lymphocyte TIL population is measured following application of an antibody or antigen-binding fragment thereof as described herein to a human tumour such as a human renal cell carcinoma. Optionally, the observed change in number or phenotype of either the γδ+ lymphocyte TIL population or non-γδ lymphocyte TIL population can then be compared to the change observed when an alternative comparator antibody is applied (e.g. OKT-3) to said model system.

Hence in another aspect of the disclosure, there is provided a method of assessing an antibody or antigen-binding fragment thereof which binds to the Vδ1 chain of a γδ TCR comprising administering the antibody or antigen-binding fragment thereof to TILs located in, or derived from, a human tumour and determining the effect on the number of TILs. The effect can be determined/measured over a period of time and optionally compared to the TIL number observed when said antibody is not applied over the same period of time. The effect may be an increase in the number of TILs. For example, the antibody may increase the number of TILs more than about 10%, more than about 20%, more than about 30%, more than about 40%, more than about 50%, more than about 70%, more than about 80%, more than about 90%, more than about 100% relative to the number of TILs observed when said antibody is not applied. In a further aspect, the TILs in which the number observed are γδ+ lymphocyte TIL cells and/or non-γδ lymphocyte TIL cells.

In a further aspect of the disclosure there is provided a method of selecting or characterizing or comparing antibodies or antigen-binding fragment thereof as described herein which bind to the Vδ1 chain of a γδ TCR cells antibodies by applying said antibodies to TIL or TILs located in or derived from a human tumour and then measuring the change in number of TIL or TILs cells over a period of time.

Medicaments that Modulate Human Vδ1+ Cytotoxicity

An antibody or antigen-binding fragment thereof as described herein may also be assessed by measuring the conferred effect on Vδ1+ mediated cell cytotoxicity. Surprisingly, during such assessments of antibodies as described herein measurably enhanced Vδ1+ mediated cell cytotoxicity was observed. For example, a reduction in the number of cancer cells or an increase in the number of killed cancer cells is observed following application of an antibody or antigen-binding fragment thereof to a model system comprising a mixed culture comprising Vδ1+ cells and said cancer cells. Optionally, the reduction in the number of cancer cells or the increase in the number of killed cancer cells can then be compared to the outcome when an alternative comparator antibody is applied (e.g. OKT-3) to said model systems.

Hence in another aspect of the disclosure, there is provided a method of assessing an antibody or antigen-binding fragment thereof which binds to the Vδ1 chain of a γδ TCR comprising applying the antibody or antigen-binding fragment thereof to a mixed population of cells comprising Vδ1+ cells and cancer cells and measuring the cytotoxicity of the Vδ1+ cells towards the cancer cells. The cytotoxicity may be measured by an increase in the number of dead cancer cells over a period of time, optionally compared to the number of dead cancer cells observed when said antibody is not applied to the mixed population of cells over the same period of time. For example, the observed increase in dead cells when said antibody is applied may be more than about 10%, by more than about 20%, by more than about 30%, by more than about 40%, by more than about 50%, more than about 70%, more than about 80%, more than about 90%, more than about 100%, more than about 200%, more than about 500%, relative to the number of dead cells observed when said antibody is not applied.

In a further aspect of the disclosure there is provided a method of selecting or characterizing or comparing antibodies or antigen-binding fragment thereof as described herein which bind to the Vδ1 chain of a γδ TCR cells by adding said antibodies to said population of mixed immune cells comprising human Vδ1+ cells and cancer cells and then measuring an increase in dead cancer cells overtime.

Medicaments which Enhance Vδ1+ Cell EC50 Cytotoxicity

An alternate way to measure the observed enhanced cytotoxicity of human Vδ1+ cells or population thereof is to measure the number of cells required to kill 50% of the cancer cells over a set period of time in condition A (such as starting control) and compare this to the number of cells required to kill 50% of the cancer cells over a set period of time in condition B (such as upon application of antibody of the disclosure as described herein).

Whilst it is recognized that there are a variety of ways to measure such parameters, to aid in understanding, the following non-limiting hypothetical example will be outlined:

Hypothetically, effector cell cytotoxicity enhancement can be measured as follows: In condition A (control treatment) it is observed that 1000 Vδ1+ cells are required to kill 50% of the cancer cells over a set period of time (e.g. 5 hours). In condition B (e.g. application of antibody of the disclosure described herein) it is observed that 500 Vδ1+ cells were required to kill 50% of the cancer cell over the same period of time. Hence in this example, the application of an antibody has enhanced the cytotoxicity of the Vδ1+ cell population by 200%:

$$( 1000 / 500 ) \times 100 = 200 \%\%$$

For example (see e.g. Examples 19 to 21), surprisingly such percentage enhancements have been observed for multispecific antibodies of the disclosure as described herein.

In a further aspect of the disclosure there is provided a method of selecting or characterizing or comparing antibodies or antigen-binding fragment thereof as described herein which bind to the Vδ1 chain of a γδ TCR by adding said antibodies to said population of mixed immune cells comprising Vδ1+ cells and cancer cells and determining the relative or percent-change in cytotoxicity versus an equivalent or control experiment wherein there is no application of said antibody to said mixture of cells.

Medicaments which Enhance Vδ1+ Cells Diseased-Cell Specificity Whilst Sparing Healthy Cells

Another approach to assess multispecific antibodies or antigen-binding fragments thereof as described herein is to measure how said antibodies modulate diseased-cell specific cytotoxicity. Surprisingly during such studies, it was discovered such antibodies can specifically enhance the Vδ1+ cell specific killing of diseased cells such as cancer cells whilst sparing healthy or non-diseased cells. Ideal antibody medicaments administered to a patient to ameliorate a symptom of cancer will confer enhanced cytotoxicity specifically towards diseased cells whilst sparing healthy cells. And medicaments which enhance effector cell cytotoxicity specifically towards diseased cells, such as cancer cells, can be said to exhibit an enhanced therapeutic index (TI) over medicaments which do not selectively enhance effector cell cytotoxicity specifically towards said diseased cells. The therapeutic index is also referred to as therapeutic ratio and is a quantitative measurement of the relative safety of a drug. It is a comparison of the amount of a therapeutic agent that causes the therapeutic effect to the amount that causes toxicity e.g. by conferring undesirable death in related or relevant healthy cell populations. An antibody or antigen-binding fragment thereof as described herein may be assessed by measuring its ability to change or to enhance or to fold-improve Vδ1+ cell capacity to selectivity kill diseased cells over and above healthy cells in model systems. For example, said model systems may comprise Vδ1+ effector cells, cancer cells, and control cells (such as healthy cells). Optionally, the fold-improvement in selective diseased-cell killing conferred by multispecific antibodies of the disclosure can then be compared to the fold-improvement observed when an alternative comparator antibody is applied (e.g. OKT-3) to said model systems.

The diseased-cell specificity and diseased-cell specificity-enhancement of Vδ1+ cells can be measured in cultures comprising Vδ1+ cells, diseased cells, and healthy cells. For example, Vδ1+ specificity towards diseased cells can be measured by observing the number of cancer cells killed by the Vδ1+ cells and then comparing the number of healthy cells killed by the Vδ1+ cells. Such comparisons can be controlled by including equivalent numbers of diseased cells and healthy cells in a model system also containing Vδ1+ cells e.g. “tricultures”. Alternative comparison methodology can also be considered—for example when analytical or equipment limitations reduce the ability to distinguish and track all three cell types or more in parallel in a single assay (inclusive of Vδ1+ cells, diseased cells, and non-diseased cells). In said instances, comparing Vδ1+ cell cytotoxicity towards diseased cells in one experiment and then comparing Vδ1+ cell cytotoxicity towards healthy cells in a separate equivalent experiment offers an alternate approach to such studies.

In another aspect of the disclosure, there is provided a method of assessing an antibody or antigen-binding fragment thereof which binds to the Vδ1 chain of a γδ TCR comprising administering the antibody or antigen-binding fragment thereof to a cell population comprising Vδ1+ cells and target cells and measuring the cell cytotoxic specificity towards the target cells. In one embodiment, the cell cytotoxicity specificity to a first target cell type can be compared to the cytotoxicity observed towards a second target cell type, therefore the method may be repeated using different target cell types. In a further aspect of the disclosure the first target cell type is a diseased cell, and the second target cell type is a control cell such as a healthy cell or a cell with a different disease to the first target cell type.

In a further aspect of the disclosure there is provided a method for selecting or characterizing or comparing antibodies or antigen-binding fragment thereof as described herein which bind to the Vδ1 chain of a γδ TCR wherein the effect conferred by said antibody on Vδ1+ cell cytotoxicity towards (i) a first cell type and (ii) a second cell type is measured and compared. In a further aspect of the disclosure an antibody is thereby selected which enhances the specific cytotoxicity towards the first cell type more so than towards the second cell type. In a further aspect of the disclosure the first cell type is a diseased-cell, and the second cell type is a healthy cell.

As described herein, the multispecific antibodies or antigen-binding fragments thereof used in the assays may be presented on a surface, for example the surface of a cell, such as a cell comprising an Fc receptor.

For example, the multispecific antibodies or antigen-binding fragments thereof may be presented on the surface of THP-1 cells, such as TIB-202™ cells (available from American Type Culture Collection (ATCC®)). Alternatively, the multispecific antibodies or antigen-binding fragments thereof may be used directly in the assays.

In such functional assays, output may be measured by calculating the half maximal concentration, also referred to as “EC50” or “effective concentration at 50 percent”. The term “IC50” refers to the inhibitory concentration. Both EC50 and IC50 may be measured using methods known in the art, such as flow cytometry methods. In some instances, EC50 and IC50 are the same value or can be considered equivalent. For example, the effective concentration (EC) of effector cells required to inhibit (e.g. kill) 50% of a certain cell type may also be considered the 50% inhibitory concentration (IC). For the avoidance of doubt, the values of EC50 in the present application are provided using IgG1 formatted antibody when referring to an antibody. Such values can be easily converted based on the molecular weight of the antibody format for equivalent values as follows:

$$( μ g / ml ) / ( MW in kDa ) = μ M$$

The EC50 for downregulation of the γδ TCR upon antibody (or fragment) binding for the parental clones described herein may be less than 0.50 μg/ml, such as less than 0.40 μg/ml, 0.30 μg/ml, 0.20 μg/ml, 0.15 μg/ml, 0.10 μg/ml, 0.06 μg/ml or 0.05 μg/ml. In a preferred embodiment, the EC50 for downregulation of the γδ TCR upon antibody (or fragment) binding is less than 0.10 μg/ml. In particular, the EC50 for downregulation of the γδ TCR upon antibody (or fragment) binding may be less than 0.06 μg/ml, such as less than 0.05 μg/ml, 0.04 μg/ml or 0.03 μg/ml. In particular, said EC50 values are when the antibody is measured in an IgG1 format. For example, the EC50 γδ TCR downregulation value can be measured using flow cytometry.

The EC50 for γδ T cell degranulation upon antibody (or fragment) binding may be less than 0.050 μg/ml, such as less than 0.040 μg/ml, 0.030 μg/ml, 0.020 μg/ml, 0.015 μg/ml, 0.010 μg/ml or 0.008 μg/ml. In particular, the EC50 for γδ T cell degranulation upon antibody (or fragment) binding may be less than 0.005 μg/ml, such as less than 0.002 μg/ml. In a preferred embodiment, the EC50 for γδ T cell degranulation upon antibody (or fragment) binding is less than 0.007 μg/ml. In particular, said EC50 values are when the antibody is measured in an IgG1 format. For example, the γδ T cell degranulation EC50 value can be measured by detecting CD107a expression (i.e. a marker of cell degranulation) using flow cytometry (e.g. as described in the assay of Example 7). In one embodiment, CD107a expression is measured using an anti-CD107a antibody, such as anti-human CD107a BV421 (clone H4A3) (BD Biosciences).

The EC50 for γδ T cell killing upon the antibody (or fragment) binding may be less than 0.50 μg/ml, such as less than 0.40 μg/ml, 0.30 μg/ml, 0.20 μg/ml, 0.15 μg/ml, 0.10 μg/ml or 0.07 μg/ml. In a preferred embodiment, the EC50 for γδ T cell killing upon the antibody (or fragment) binding is less than 0.10 μg/ml. In particular, the EC50 for γδ T cell killing upon the antibody (or fragment) binding may be less than 0.060 μg/ml, such as less than 0.055 μg/ml, in particular less than 0.020 μg/ml. In particular, said EC50 values are when the antibody is measured in an IgG1 format. For example, the EC50 γδ T cell killing value can be measured by detecting proportion of dead cells (i.e. using a cell viability dye) using flow cytometry following incubation of the antibody, γδ T cell and target cells (e.g. as described in the assay of Example 8). In one embodiment, death of the target cell is measured using a cell viability dye is Viability Dye eFluor™ 520 (ThermoFisher).

In the assays described in these aspects, the antibody or antigen-binding fragment thereof may be presented on the surface of a cell, such as a THP-1 cell, for example TIB-202™ (ATCC®). The THP-1 cells are optionally labelled with a dye, such as CellTracker™ Orange CMTMR (Thermo Fisher Scientific®).

Medicaments that Downregulate CD3 Molecules Associated with a Vδ1 TCR.

Presented herein are antibodies which engage the T-cell/CD3 complex differently. Specifically, these antibodies can engage via the TRDV1 domain of Vδ1 TCRs expressed only on Vδ1+ cells. In doing so such medicaments function differently. In turn this engagement event can downregulate TCR-associated CD3 molecule complexes. Such CD3 downregulation can be synonymous with T-cell activation. However, by engaging the T-cell/CD3 complex via the TRDV1 domain in this way, only CDR3 molecules associated with the Vδ1 TCR are then downregulated. This mechanism is clearly shown in FIGS. 26A-26B.

Hence in one embodiment there is provided a method of downregulating a TRDV1-containing Vδ1 TCR and the associated CD3 molecule complex on the surface of a cell with an antibody, and the use of such antibodies for this purpose.

In some embodiments, also presented herein are multispecific TCEs capable of engaging the T-cell/CD3 complex via the TRDV1 domain. Current multispecific TCE-formatted medicaments typically engage and activate a T-Cell via CD3 binding events. This can result in downregulation of CD3 molecule complexes from the surface of a T-cell. However, it also well understood that TCEs can also over-stimulate T-cells via such engagement and downregulation. CD3 molecule complexes are not specific to one class of T-cell and are therefore not a precise target to aim for. Stimulating all T-cells (mainly αβ subtype) via CD3 can in turn can result in overproduction of cytokines, leading to acute cytokine flares (so-called cytokine storms). Additionally, in non-targeted approaches which engage and activate all T-cells via CD3, paradoxically the T-cells can become overactivated which leads to chronic T-cell exhaustion and/or T-cell death. Indeed, this non-specific pan T cell activation leads to activation of both effector and regulatory T cells whereas the presently presented approach interacts specifically with an effector population. This ‘sledgehammer’ approach is therefore far from ideal when one may wish to upregulate selective T-cells only.

By contrast, presented herein are multispecific antibodies, in particular T-cell engagers, which engage the T-cell/CD3 complex differently. Specifically, these TCEs can engage via the TRDV1 domain of Vδ1 TCRs expressed only on Vδ1+ cells. In doing so such TCE-based medicaments function differently. First, these TCEs are able to down-regulate a TCR via engaging a TRDV-1 epitope. In turn this engagement event downregulates TCR-associated CD3 molecule complexes. Such CD3 downregulation can be synonymous with T-cell activation. However, by engaging the T-cell/CD3 complex via the TRDV1 domain in this way, only CDR3 molecules associated with the Vδ1 TCR are then downregulated. This approach of specifically targeting and activating Vδ1 cells allows many of the above issues (cytokine storms, T-cell exhaustion/depletion and ADCC, for example) to be avoided. Again, this mechanism is clearly shown in FIGS. 26A-26B.

Stimulation of T-cells via the CD3 ‘sledgehammer’ approach can also contribute to depletion of T-cells via Fc gamma receptor driven mechanisms, such as ADCC. Therefore, the majority of the CD3-targeting bispecific antibodies currently in clinical practice have Fc domains with reduced binding activity to FcγR or are bispecific fragments intentionally without the Fc region. CD3-targeting therapies may also have reduced binding affinity of the T-cell receptor complex binding arms.

Additionally, in the approach presented here, the antibodies engage on the TCR of vδ1 cells, but full activation does not occur unless tumour cells are also present. Full engagement of the presently presented antibodies on the TCR leads to partial downregulation and the vδ1 cells bound by the presently presented antibodies only become fully activated and become cytotoxic in the tumour microenvironment. This represents another vital safety advantage for the present approach, since off target cytotoxicity is reduced and the full potency of the present antibodies to activate vδ1 cells is only unleashed in the presence of tumour cells.

One mechanism behind γδ T cells being able to detect stress signals on tumour cells is believed to be due to the NCRs (natural cytotoxicity receptors) they express. The NCRs are able to engage NCR ligands on tumour cells. A dual mechanism of activation is therefore employed, wherein the γδ T cells are activated via TCR stimulation and the NCRs sense the tumour cells to enable full activation and cytotoxicity.

This is in contrast to stimulation of αβ T cells via CD3, wherein all stimulation is via the TCR. Such cells are therefore almost indiscriminate between healthy or transformed cells because they don't have this antigen presentation independent sensing of tumour cells via NCRs. Therefore, if CD3 antibodies are Fc enabled they will attract other immune cells which can trigger a cascade of unpredictable and desirable events such as cytokine storms, exhaustion and even overactivation of immune cells leading to, for example, NK cells killing T cells etc. In the present approach, stimulation of γδ T cells with the presently presented antibodies does not lead to such concerns because both γδ T cells (and other immune cells such as NK cells) are able to distinguish between healthy cells and tumour cells including via their NCR sensing mechanism and therefore selectively kill stressed cells such as cancer cells or virally infected cells due to this diseased cell specificity

For example, in initial cynomolgus studies with the anti-vδ1 Fc-enabled multispecific antibody presented herein, with specificity to both human and cyno vδ1 antigen (SEQ ID NO: 1 and SEQ D NO: 172), was found to be safe and well tolerated, in dose-escalating, repeat dose in-vivo studies by all parameters measured. None of the side effects typically associated with T cell activation, such as cytokine release or weight loss were observed.

These findings also highlight another advantage of the approach described herein. Specifically, and unlike TCEs typified by CD3 engagers and the like, TCE bispecifics of the disclosure can optionally be designed as full-length antibody, for example comprising heavy chains with a VH-CH1-CH2-CH3 format with cognate light chains with a VL-CL format. Unlike smaller bispecific formats (e.g. less than 70 KDa), such full-length bispecific format can exhibit longer half-lives in-vivo and thereby require less frequent dosing regimens. The longer half-lives observed by such formats are for a variety of reasons inclusive of increased size (>70 KDa) which means such formats are not filtered by kidneys (glomerulus pore size cut-off 60-70 KDa). In one embodiment, the multispecific antibodies may be larger than about 70 KDa, and may comprise human IGHC sequence (e.g. IGHA, IGHD, IGHD, IGHM, IGHG sequence) as listed by IMGT. Such IgG1 formats can also be re-cycled by FcRn mechanism. The clear downside of such full-length antibody formats for TCE bispecifics in particular is that such format exhibit unfavourable safety profiles due to the reduce clearance rate, increased and more chronic exposures.

Therefore, the present approach allows the possibility of Fc functionality without any concern of off-target effects, such as NK cells killing γδ T cells or vice versa. The present approach is therefore superior to CD3 directed approaches which are stunted by the necessity of workarounds such as reducing CD3 affinity, eliminating Fc function etc. to limit collateral damage outside the tumour environment. The multispecific antibodies, in particular T-cell engagers, presented here are able to bind to vδ1 cells without any damage potential and the full activation and cytotoxicity enhancement is only engaged when the vδ1 cells are in close contact with tumour cells.

Hence in one embodiment there is provided a method of downregulating a TRDV1-containing Vδ1 TCR and the associated CD3 molecule complex on the surface of a cell with a TCE multispecific antibody, and the use of such multispecific antibodies for this purpose.

Other features and advantages of the present disclosure will be apparent from the description provided herein. It should be understood, however, that the description and the specific examples while indicating preferred embodiments of the disclosure are given by way of illustration only, since various changes and modifications will become apparent to those skilled in the art. The disclosed technology will now be described using the following, non-limiting examples:

### EXAMPLES

### Example 1: Tables

Parental antibody clones were prepared as set out in Examples 1 to 19 in WO2021/032963.

The sequences of the CDRs of the parental clones are shown below in Table 8.

TABLE 7
DV1 binders for IgG conversion

| | | SEQ | | SEQ | | SEQ | | SEQ | | | | SEQ | |
|---|---|---|---|---|---|---|---|---|---|---|---|---|---|
| | | ID | | ID | | ID | | ID | Light | | | ID | 100 nM |
| Clone ID | Heavy CDR1 | NO. | Heavy CDR2 | NO | Heavy CDR3 | NO. | Light CDR1 | NO | CDR2 | SEQ | Light CDR3 | NO. | L1 |
| 1245_P02_G04 | GDSVSSKSAA | 51 | TYYRSKWST | 53 | TWSGYVDV | 54 | QDINDW | 79 | DAS | 80 | QQSYSTPQVT | 81 | 5896 |
| 1245_P01_E07 | GFTFSDYY | 130 | ISSSGSTI | 131 | VDYADAFDI | 132 | QSIGTY | 144 | VAS | 145 | QQSYSTLLT | 146 | 162591 |
| 1252_P01_C08 | GFTVSSNY | 317 | IYSGGST | 318 | PIELGAFDI | 319 | NIGSQS | 347 | YDS | 348 | QVWDSSSDHVV | 349 | 1977 |
| 1245_P01_B07 | GFTFSDYY | 320 | ISSSGSTI | 321 | ENYLNAFDI | 322 | QSLSNY | 350 | AAS | 351 | QQSYSTPLT | 352 | 64271 |
| 1251_P02_C05 | GFTFSSYA | 323 | ISGGGGTT | 324 | DSGVAFDI | 325 | QNIRTW | 353 | DAS | 354 | QQFKRYPPT | 355 | 65269 |
| 1139_P01_E04 | GDSVSSNSAA | 326 | TYYRSKWYN | 327 | SWNDAFDI | 328 | QSISTW | 356 | DAS | 357 | QQSYSTPLT | 358 | 23786 |
| 1245_P02_F07 | GDSVSSNSAA | 329 | TYYRSKWYN | 330 | DYYYSMDV | 331 | QSISSW | 359 | DAS | 360 | QQSHSHPPT | 361 | 10450 |
| 1245_P01_G06 | GFTFSDYY | 332 | ISSSGSTI | 333 | HSWNDAFDV | 334 | QSISSY | 362 | AAS | 363 | QQSYSTPDT | 364 | 22474 |
| 1245_P01_G09 | GDSVSSNSAA | 335 | TYYGSKWYN | 336 | DYYYSMDV | 331 | QSISTW | 365 | DAS | 366 | QQSYSTPVT | 367 | 18430 |
| 1138_P01_B09 | GFTFSDYY | 338 | ISSSGSTI | 339 | HSWSDAFDI | 340 | QDISNY | 368 | DAS | 369 | QQSYSTPLT | 370 | 29193 |
| 1251_P02_G10 | GFTFSDYY | 341 | ISSSGSTI | 342 | HSWNDAFDI | 343 | QSISSH | 371 | AAS | 372 | QQSYSTLLT | 373 | 17053 |
| 1141_P01_E01 | GYSFTSYW | 344 | IYPGDSDT | 345 | HQVDTRTADY | 346 | RSDVGGYNY | 374 | EVS | 375 | SSYTSTSTLV | 376 | 136780 |

The K_{D} values of the parental clones, as determined by SPR analysis, was as follows:

TABLE 8
Results of IgG capture

| Analyte | Clone ID | K_{D} (nM) | K_{D} (M) |
|---|---|---|---|
| L1 (DV1-GV4) | 1245_P01_E07 | 12.4 | 1.24e−08 |
| L1 (DV1-GV4) | 1252_P01_C08 | 100 | 1.00e−07 |
| L1 (DV1-GV4) | 1245_P02_G04 | 126 | 1.26e−07 |
| L1 (DV1-GV4) | 1245_P01_B07 | 341 | 3.41e−07 |
| L1 (DV1-GV4) | 1251_P02_C05 | 1967* | 1.97e−06 |
| L1 (DV1-GV4) | 1139_P01_E04 | 251 | 2.51e−07 |
| L1 (DV1-GV4) | 1245_P02_F07 | 193 | 1.93e−07 |
| L1 (DV1-GV4) | 1245_P01_G06 | 264 | 2.64e−07 |
| L1 (DV1-GV4) | 1245_P01_G09 | 208 | 2.08e−07 |
| L1 (DV1-GV4) | 1138_P01_B09 | 290 | 2.90e−07 |
| L1 (DV1-GV4) | 1251_P02_G10 | 829 | 8.29e−07 |
| L1 (DV1-GV4) | TS8.2 (commercial anti-Vδ1 | 44 | 4.40e−08 |
| | antibody) | | |

*Binding of 1252_P02_C05 did not reach saturation, therefore data was extrapolated

Epitope mapping was conducted for 5 of the parental clones, using hydrogen-deuterium exchange. A summary of the epitope mapping results is presented in Table 29.

TABLE 29
Results of epitope mapping for antigen/antibody complexes

| | Epitope mapping, amino acid |
|---|---|
| Clone ID | numbering of SEQ ID NO: 272 |
| 1245_P01_E07 | 5, 9, 16, 20, 62, 64, 72, 77 |
| 1252_P01_C08 | 50, 53, 59, 62, 64 |
| 1245_P02_G04 | 37, 42, 50, 53, 59, 64, |
| | 68, 69, 72, 73, 77 |
| 1251_P02_C05 | 59, 60, 68, 72 |
| 1141_P01_E01 | 3, 5, 9, 10, 12, 16, |
| | 17, 62, 64, 68, 69 |

### Example 3: Mammalian Display

Two clones were selected for affinity maturation. The above examples describe the preparation and characterization of affinity matured clones derived from clone ADT1-4 (G04) and clone ADT1-7 (E07).

### Affinity Maturation of Human Anti-Vδ1 Monoclonal Antibodies

Phage display employed to generate parental anti Vδ1 monoclonal antibodies yielded antibodies with the affinity ranging from 10 nM-1 pM, as described above. Parental antibodies clone ADT1-4 (G04) and clone ADT1-7 (E07) were then affinity matured in vitro to surprisingly attain 100-fold improved affinity for superior target engagement. In vitro affinity maturation of parental antibodies was achieved via two-step process: diversification of the parental antibody sequence using targeted CDR3 mutagenesis and then selective enrichment of affinity improved antibodies using phage and mammalian display platforms. VH and VL CDR3 2-mer libraries for the clones ADT1-4 and ADT1-7 were created using Kunkel mutagenesis (Kunkel et al., 1987; Sidhu and Weiss, 2004) and RCA amplification. Combinations of all single and double amino acid substitutions at specified positions in the VH and VL CDR3s were incorporated using Agilent primer synthesis technology. The number of different amino acids to be incorporated at a particular position were specified. Cysteine and methionine were omitted. The changes made in the CDRs of the clones are summarized below in Table 30.

TABLE 30
CDR3 positions and specified residue changes

| | POS | AMINOACID(S) | POS | AMINOACID(S) |
|---|---|---|---|---|
| E07 | V | [symbol] | Q | [symbol] |
| (V3-11/Vκ1-39) | D | [symbol] | [symbol] | [symbol] |
| | [symbol] | [symbol] | [symbol] | [symbol] |
| | A | [symbol] | [symbol] | [symbol] |
| | D | [symbol] | T | [symbol] |
| | A | [symbol] | L | [symbol] |
| | F | [symbol] | L | [symbol] |
| | D | [symbol] | [symbol] | [symbol] |
| | I | [symbol] | | |
| G04 | T | [symbol] | [symbol] | [symbol] |
| (VH6-1/Vκ1-5) | W | [symbol] | [symbol] | [symbol] |
| | S | [symbol] | [symbol] | [symbol] |
| | [symbol] | [symbol] | T | [symbol] |
| | [symbol] | [symbol] | P | [symbol] |
| | [symbol] | [symbol] | Q | [symbol] |
| | D | [symbol] | [symbol] | [symbol] |
| | [symbol] | [symbol] | [symbol] | [symbol] |

[symbol]

indicates data missing or illegible when filed

The size of each library is shown in Table 27.

TABLE 31
Library Size

| | |
|---|---|
| ADT1-7 | 5.4 × 10⁹ |
| ADT1-4 | 5.5 × 10⁹ |

### Generation of Mutagenized VH and VL CDR3 Libraries

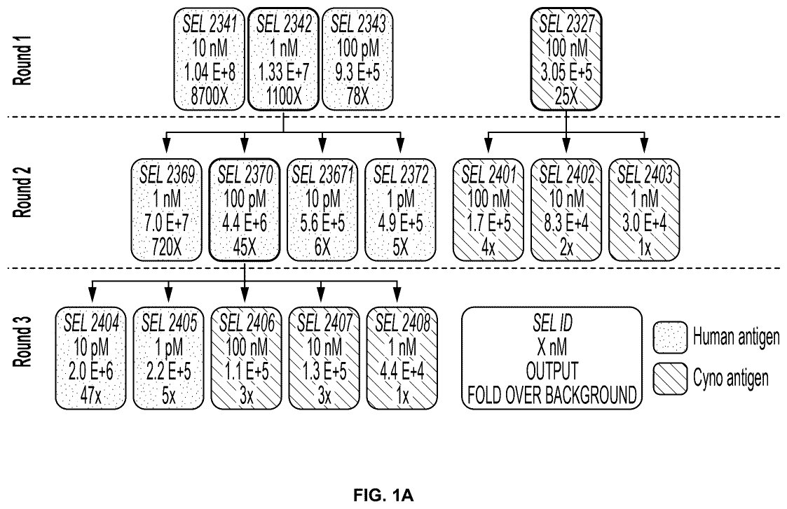

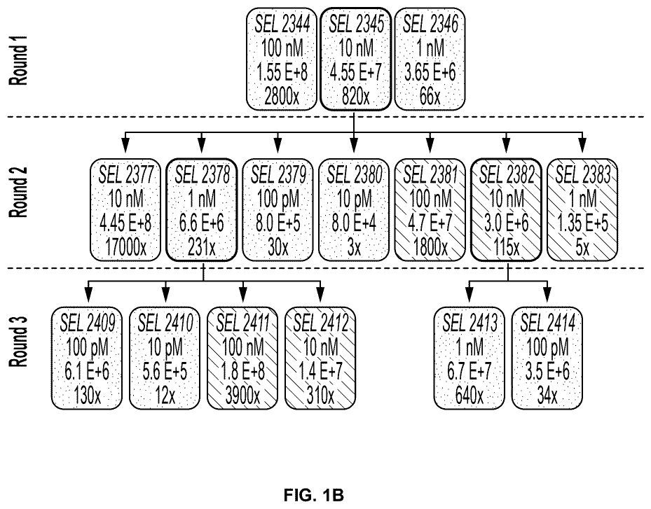

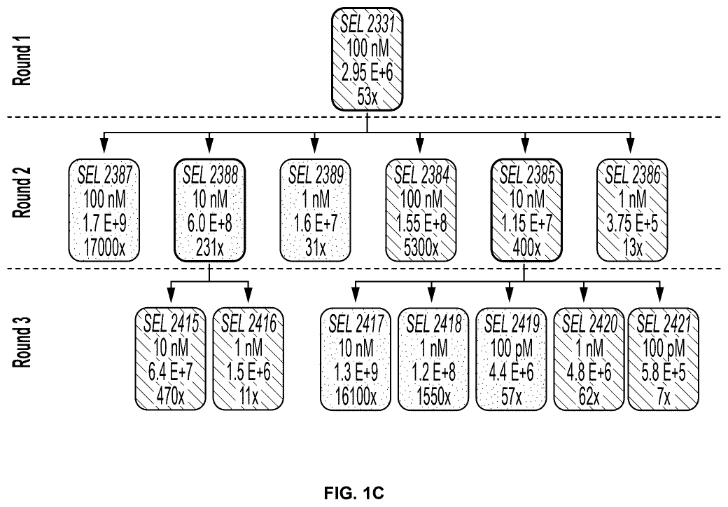

Mutagenized libraries were prepared from suitable culture volumes covering an appropriate excess of library members. Affinity maturation was performed using phage display technology as described in Schofield et al. (2007) using solution phase selection. Selections were performed in the solutions using human and cyno antigen. To isolate binders from a mutant library with higher affinities antigen concentration was controlled to give a series of increasingly stringent selections. Several rounds of phage display selections with human and with cyno antigen were performed for each clone as presented in the FIGS. 1A-1C. For ADT1-4 lineage the antigen concentration used in the selection was ranging from 100 nm-10 pM for human and 100 nM and 10 nM for cyno antigen. For ADT1-7 lineage the human antigen was used at concentration from 10 nM to 1 pM and for cyno from 100 nM to 100 pM. FIGS. 1A-1C show the phage selection rounds for the ADT1-7 library (iA), the ADT1-4 library (iB) and the ADT1-7 library with a selection strategy for isolating cyno cross-reactive binders (iC).

Polyclonal phage ELISA was used to evaluate the progress of the selections. Human DV1/GV4 and Cyno DV1/GV76 were coated overnight at 150 ng/well, 50 μL/well. Cyno DV2/GV76 and HSA were used as controls. Only the outputs from the ADT4-1 library were both cross reactive to human and cyno DV1 antigen. The output from ADT1-7 were only reactive to human antigen. Selected outputs were characterised further by monoclonal phage ELISA and the summary is presented in Table 32. The italicized and bolded text indicates the selection was carried out with either human or cyno antigen, respectively. The arrows provide an indication as to the percentage of clones that are classified as binders to human or cyno VD1 (pointing up being high and pointing down being low).

TABLE 32
Monoclonal phage ELISA summary

| G04 | phage rounds | | | Binding | |
|---|---|---|---|---|---|
| selection | #1 | #2 | #3 | huDV1 | cyDV1 |
| 2378 | 10 nM | 1 nM | | ↑ 79% | [symbol] 25% |
| 2409 | | | 100 pM | ↑ 75% | ↑ 100% |
| 2412 | | | 10 nM | [symbol] 72% | ↑ 100% |
| 2413 | | 10 nM | 1 nM | ↑ 84% | ↑ 100% |
| 2385 | 100 nM | 10 nM | | [symbol] 71% | ↑ 75% |
| 2418 | | | 1 nM | ↑ 100% | ↑ 94% |
| 2420 | | | 1 nM | ↑ 94% | ↑ 94% |
| 2415 | | 10 nM | 10 nM | ↑ 100% | ↑ 94% |

| E07 | phage rounds | | | | |
|---|---|---|---|---|---|
| selection | #1 | #2 | #3 | huDV1 | cyDV1 |
| 2370 | 1 nM | 100 pM | | ↑ 96% | ↓ 0% |
| 2404 | | | 10 pM | ↑ 83% | ↓ 0% |

The sequence diversity is summarised in Table 33. The italicized and bolded text indicates the selection was carried out with either human or cyno antigen, respectively. The arrows provide an indication as to the level of diversity (pointing up being high and pointing down being low).

TABLE 33
Monoclonal phage sequencing summary

| G04 | phage rounds | | | | | | diversity | | |
|---|---|---|---|---|---|---|---|---|---|
| selection | #1 | | #2 | | #3 | | heavy CDR3 | light CDR3 | heavy-light CDR3 |
| 2378 | 10 | nM | 1 | nM | | | [symbol] 83% | [symbol] 96% | [symbol] 100% |
| 2409 | | | | | 100 | pM | [symbol] 50% | [symbol] 53% | [symbol] 72% |
| 2412 | | | | | 10 | nM | [symbol] 63% | [symbol] 97% | [symbol] 97% |
| 2413 | | | 10 | nM | 1 | nM | [symbol] 71% | [symbol] 97% | [symbol] 100% |
| 2385 | 100 | nM | 10 | nM | | | [symbol] 83% | [symbol] 88% | [symbol] 100% |

| | | | | | | | |
|---|---|---|---|---|---|---|---|
| 2418 | | | 1 | nM | [symbol] 66% | [symbol] 81% | [symbol] 88% |
| 2420 | | | 1 | nM | [symbol] 63% | [symbol] 73% | [symbol] 80% |
| 2415 | 10 | nM | 10 | nM | [symbol] 63% | [symbol] 78% | [symbol] 88% |

| E07 | phage rounds | | | | | |
|---|---|---|---|---|---|---|
| selection | #1 | #2 | #3 | heavy CDR3 | light CDR3 | heavy-light CDR3 |
| 2370 | 1 nM | 100 pM | | [symbol] 88% | [symbol] 96% | [symbol] 100% |
| 2404 | | | 10 pM | [symbol] 75% | [symbol] 67% | [symbol] 96% |

### Generation of IgG Mammalian Display Libraries

To form final libraries for mammalian display the following ADT1-4 selections were polled:
SEL2409, 2412 and 2413 to create ADT1-4 library 1 (human)
SEL 2418, 2420 and 2415 to create ADT1-4 library 2 (cyno)
SEL2370 and 2404 to create ADT1-7 library.

Pools were then advanced to mammalian display. Single chain variable fragment antibodies (scFvs) populations were converted en masse into the IgG format, maintaining the original variable heavy (VH) and variable light (VL) chain pairing. The IgG formatted antibodies were then cloned into a mammalian display donor vector which was co-transfected with plasmids encoding a TALE nuclease pair to enable nuclease directed antibody gene integration at a single chromosomal locus. A mammalian display antibody library covering the phage output diversity (>10⁶ clones) was created in HEK293 cells. Stable populations of cells expressing antibodies on the cell surface were selected by blasticidin addition 2 days post transfection (dpt). Cells expressing antibodies on the cell surface were enriched by magnetic-activated cell sorting (MACS) sorting (7 dpt); the cells were labelled with anti-Fc-PE followed by anti-PE microbeads and sorted using Midi MACS magnet (Miltenyi Biotec®) and LS columns. These populations of cells were advanced to selections.

### Selection of Matured Antibodies by Mammalian Display

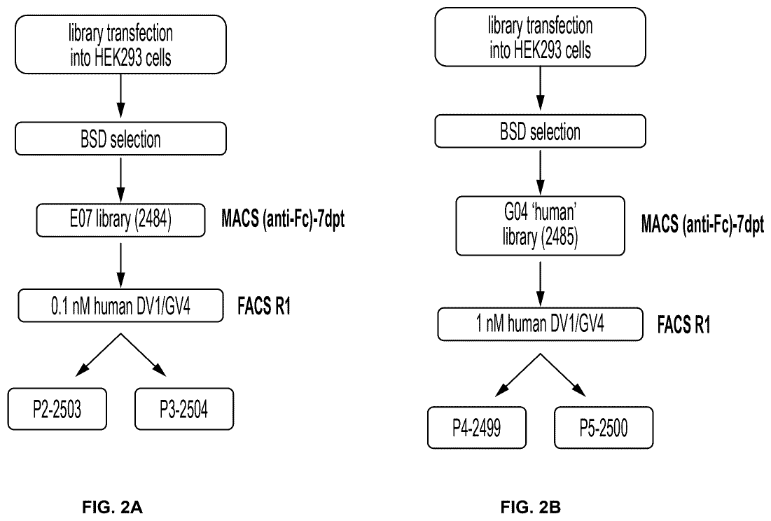

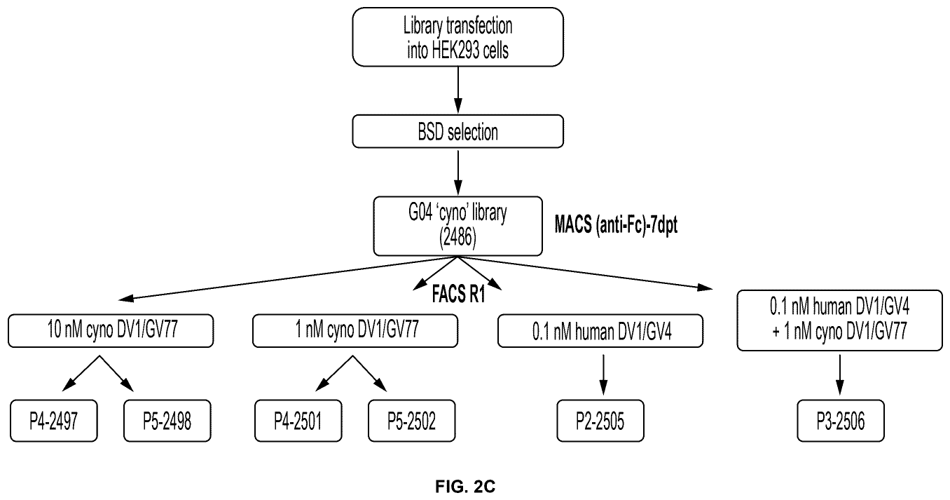

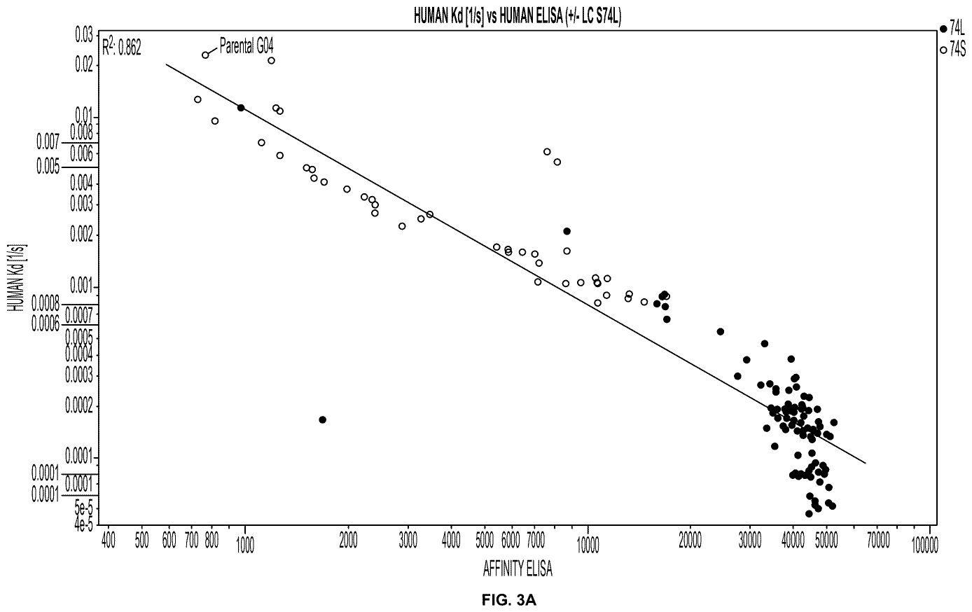

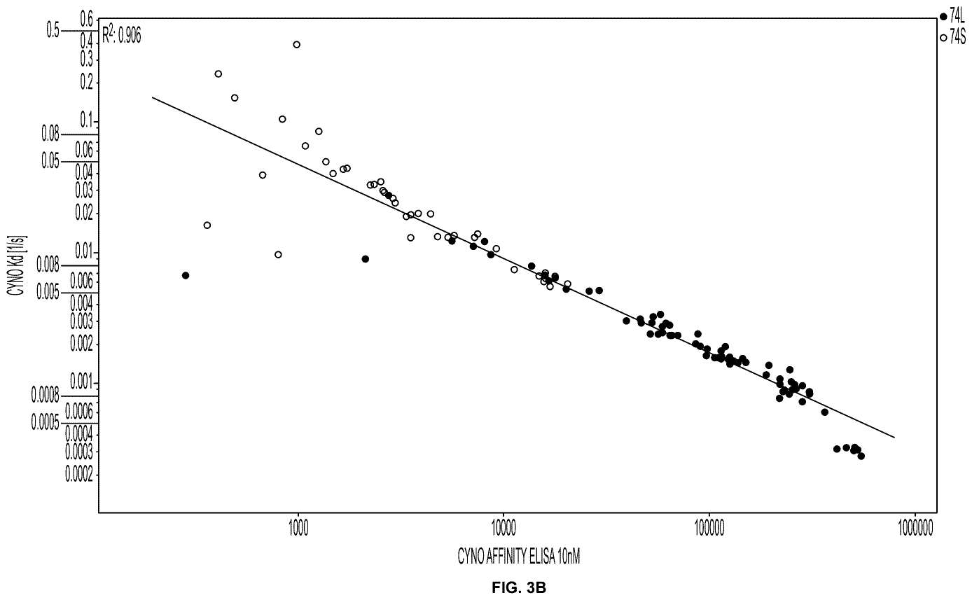

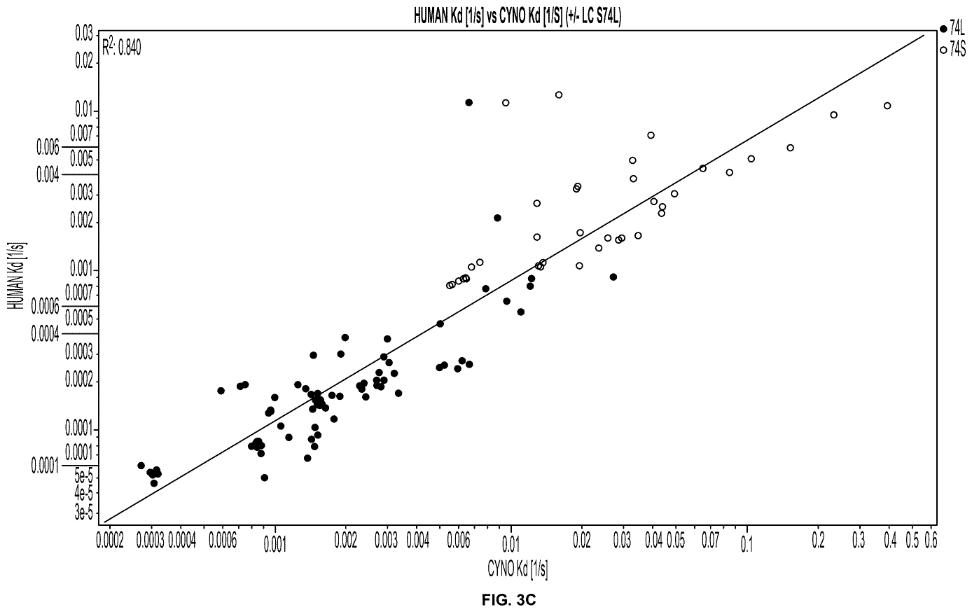

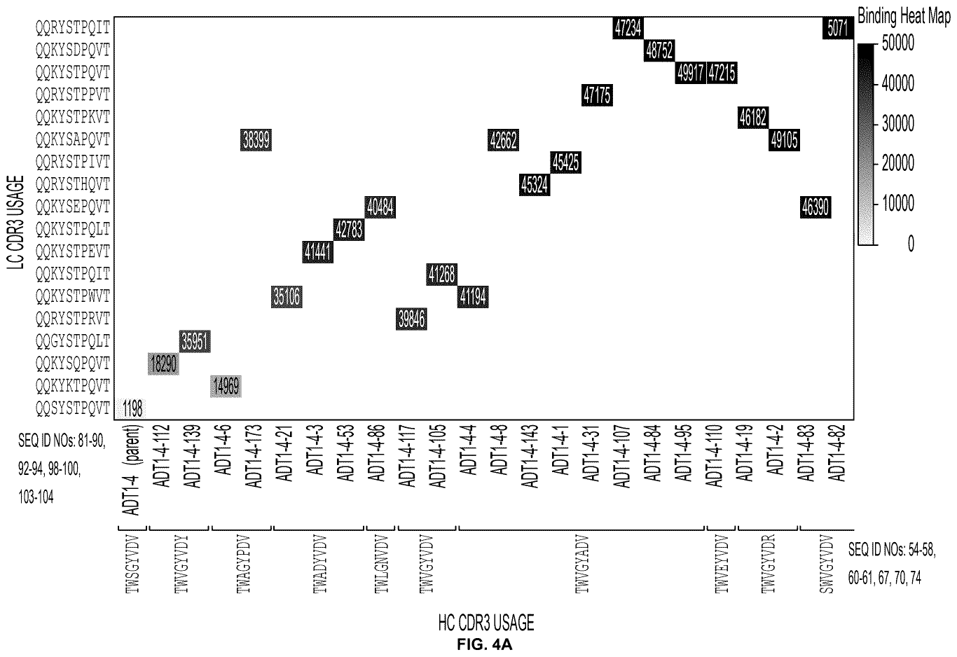

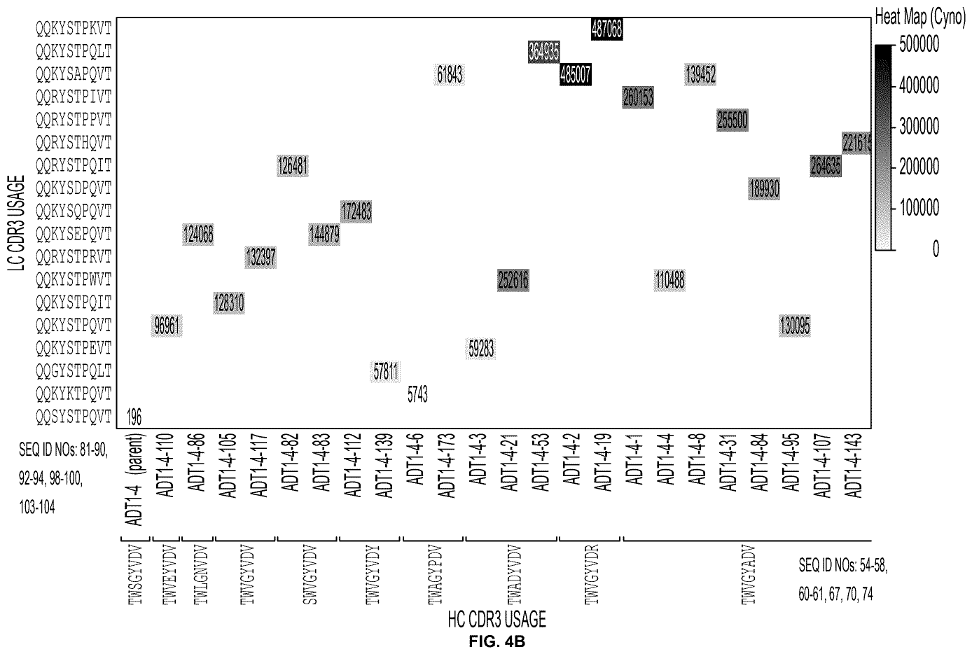

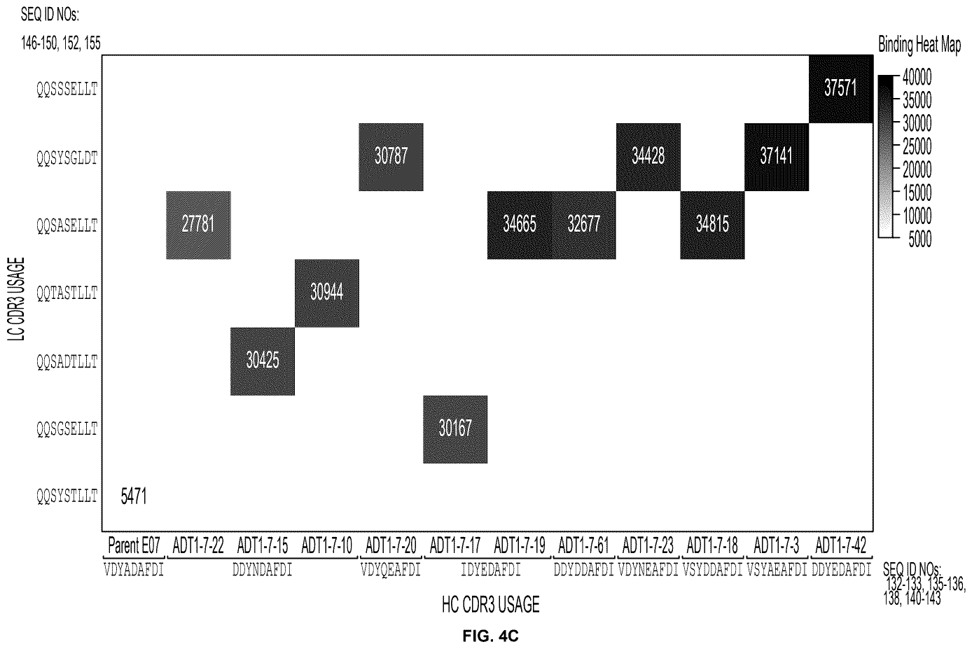

Following MACS to enrich antibody expressing cells, two strategies were adopted to identify TRDV1 binders. The main strategy involved dual colour fluorescence sorting based on Fc expression and antigen binding. The other involved dual colour fluorescence sorting based on the binding of both cyno and human antigens to maximise the chances of isolating high affinity cross-reactive binders. A schematic overview of the process is presented in FIGS. 2A-2C.

### Sequence, Specificity, Affinity Ranking and Characterisation

Genomic DNA was extracted from eight different sorted populations. DNA encoding the selected IgGs were amplified and cloned into the soluble IgG1 expression vector. A total of 1472 clones from the 8 different selections were chosen. The pDNA was transfected into Expi293 cells. Supernatants were harvested 5 days after transfection and the expressed antibodies affinity ranked for human and cyno TRDV1 binding in a capture ELISA. A total of 93 anti-DV1 antibodies from each selections strand were chosen for sequence and SPR off-rate analysis. In addition, these clones were checked by direct ELISA for binding to human TRDV1, human TRDV1 (A->V), human TRDV2, cyno TRDV1 and BSA.

### Example 4: Binding Affinity to Human and Cyno Antigens

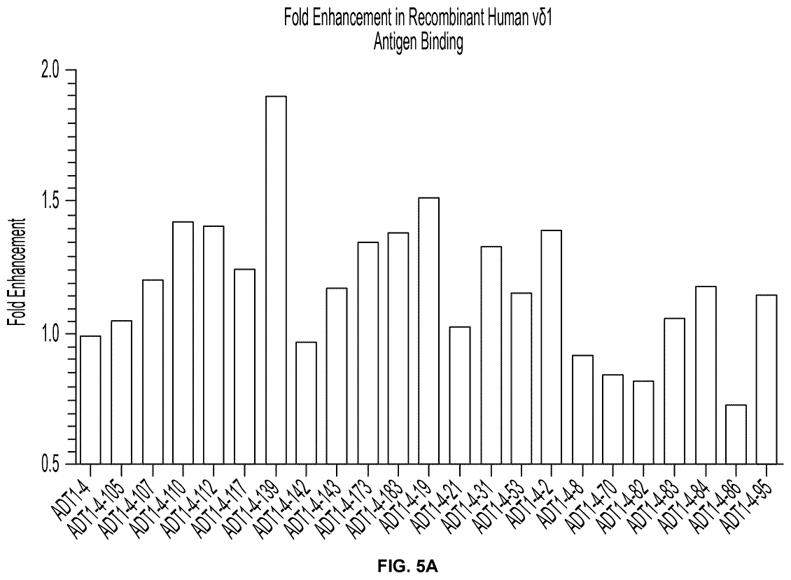

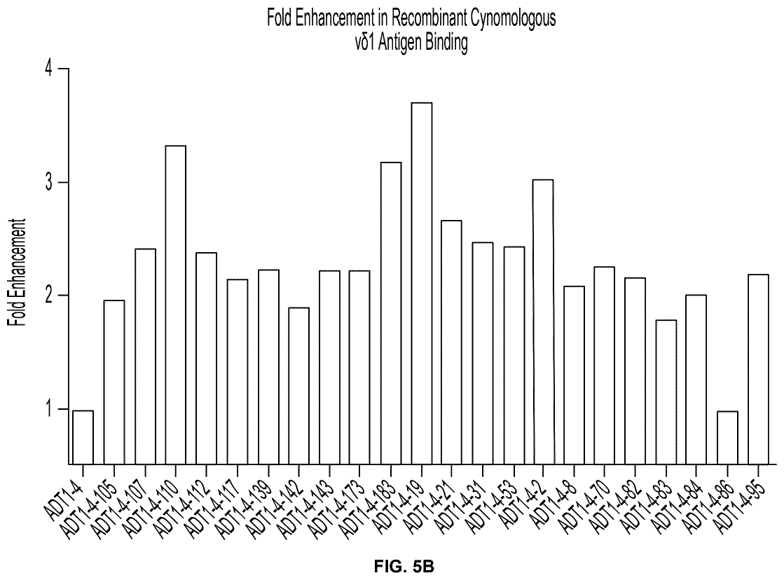

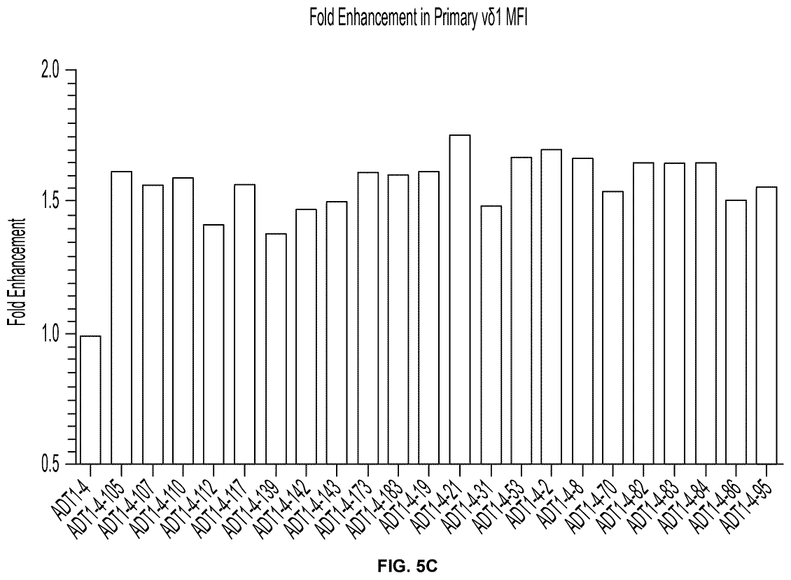

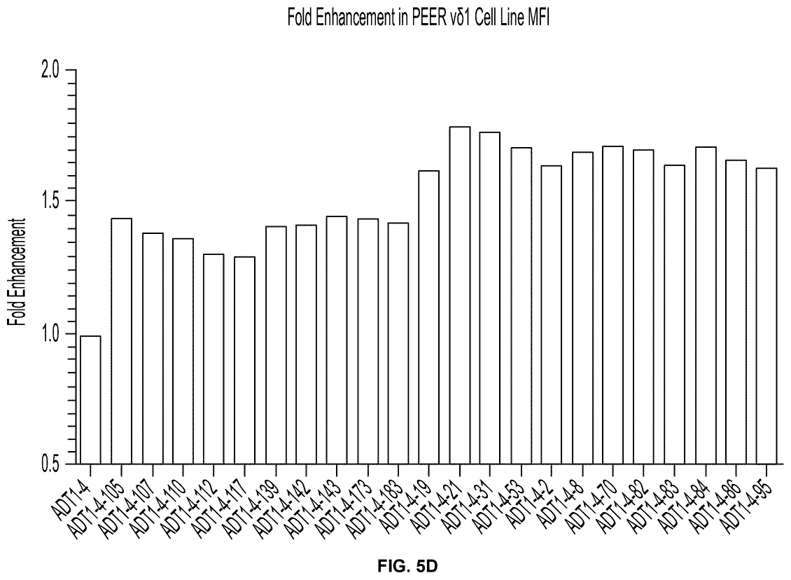

Binding to recombinant antigen and vδ1 TCR expressed on cells: ADT1-4 Lineage Studies were undertaken to explore the binding of anti-vδ1 antibodies to their target antigens. The binding of Vδ1 mAbs to Vδ1 TCR antigen was tested by ELISA. 1 μg of human antigen or 1 μg of cynomolgus antigen was immobilized per well onto 96 well Immunoassay plates (SLS #475904) and then blocked with BSA to prevent non-specific binding. 1.3 pmol (20 ng) of each mAb were added and incubated for 1 hour at room temperature. mAb binding to antigen was detected using ProteinA-HRP (Abcam® #Ab7456) along with TMB substrate (Thermo Fisher Scientific® #12750000) and Stop solution (Biolegend® #423001) by measuring absorbance at 450 nM. Parental controls were included as positive controls for both the assay and inter-plate variability. Hits were identified as those mAbs giving absorbance readings above those of the parental mAbs. FIG. 5A shows the fold increase in absorbance for matured ADT1-4 clones compared to parental ADT1-4 binding absorbance when 1.3 pmol of antibody was bound to human vδ1 antigen. FIG. 5B shows the fold increase in absorbance for matured ADT1-4 clones compared to parental ADT1-4 binding absorbance when 1.3 pmol of antibody was bound to cynomolgus vδ1 antigen.

mAb binding to endogenous Vδ1-TCR was tested using flow cytometry. Skin Vδ1 cells (donor ATS006; ADT expanded E0000113) or the PEER vδ1 cell line were seeded at 3×10\hat{ }5 cells/well into 96 well round bottom plates and resuspended in 50 ul of FACS buffer (v/v: 2% FCS, 0.1% NaAzide and 1 mM EDTA in PBS) containing 3 ug/ml of test mAb for 15 min at 4° C. Cells were pelleted and secondary anti-hmIgG-APC antibody (Miltenyi Biotec® #130-119-772) was added at 1/100 in FACS buffer and incubated for a further 20 min at 4° C. Cells were washed and fixed in CellFix (BD® #340181) and analysed by flow cytometry. The % Vδ1 phenotype and Mean Fluorescence Intensity was calculated (Inivai Technologies®, Flowlogicv7.2). As a positive control the parental antibody was included. FIG. 5C shows the fold increase in mean fluorescence intensity for matured ADT1-4 clones compared to parental ADT1-4 binding primary skin derived human vδ1 cells. FIG. 5D shows the fold increase in mean fluorescence intensity for matured ADT1-4 clones compared to parental ADT1-4 binding the transformed PEER vδ1 cell line.

Binding to Recombinant Antigen and vδ1 TCR Expressed on Cells: ADT1-7 Lineage:

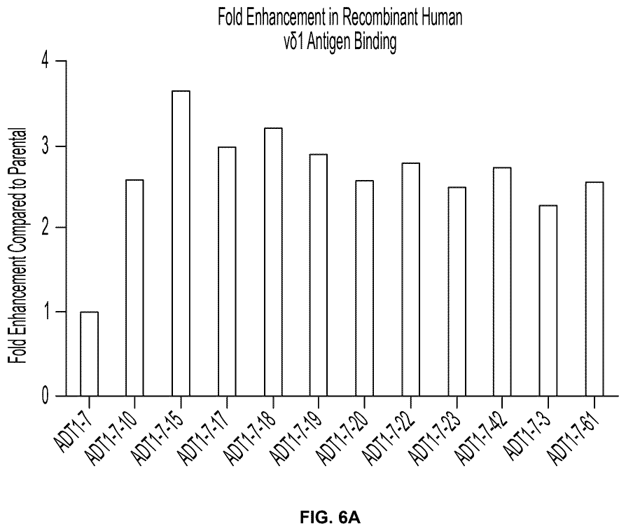

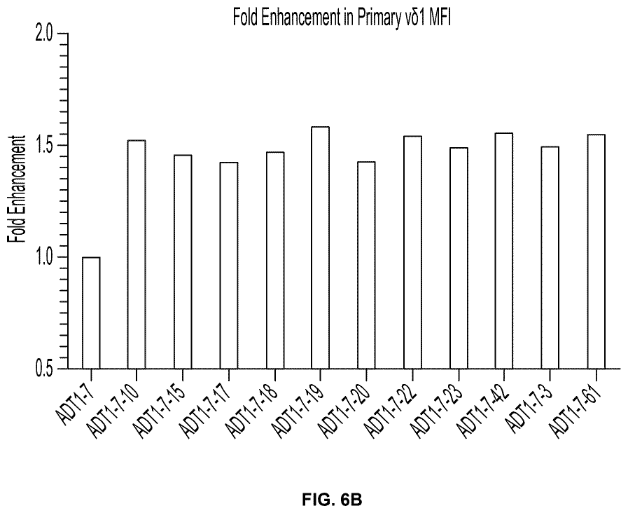

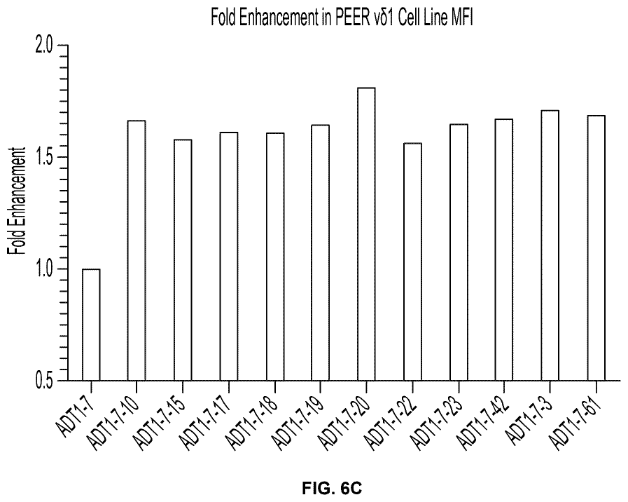

Studies were undertaken to explore the binding of ADT1-7 matured anti-vδ1 antibodies to their target antigens. The binding of Vδ1 mAbs to vδ1 TCR antigen was tested by ELISA. 1 ug of antigen was immobilized per well onto 96 well Immunoassay plates (SLS #475904) and then blocked with BSA to prevent non-specific binding. A titration of 6.7 pmol (100 ng), 1.3 pmol (20 ng) and 0.27 pmol (4 ng) of mAb were added and incubated for 1 hour at room temperature. mAb binding to antigen was detected using ProteinA-HRP (Abcam® #Ab7456) along with TMB substrate (Thermo Fisher Scientific® #12750000) and Stop solution (Biologend® #423001) by measuring absorbance at 450 nM. Parental controls were included as positive controls for both the assay and inter-plate variability. Hits were identified as those mAbs giving absorbance readings above those of the parental mAbs. FIG. 6A shows the fold increase in absorbance for matured ADT1-4 clones compared to parental ADT1-4 binding absorbance when 1.3 pmol of antibody was bound to human vδ1 antigen.

mAb binding to endogenous Vδ1-TCR was tested using flow cytometry. Skin vδ1 cells (donor ATS006; ADT expanded E0000113) or the PEER vδ1 cell line were seeded at 3×10\hat{ }5 cells/well into 96 well round bottom plates and resuspended in 50 ul of FACS buffer (v/v: 2% FCS, 0.1% NaAzide and 1 mM EDTA in PBS) containing 3 ug/ml of test mAb for 15 min at 4° C. Cells were pelleted and secondary anti-hmIgG-APC antibody (Miltenyi Biotec® #130-119-772) was added at 1/100 in FACS buffer and incubated for a further 20 min at 4° C. Cells were washed and fixed in CellFix (BD® #340181) and analysed by flow cytometry. The % Vδ1 phenotype and Mean Fluorescence Intensity was calculated (Inivai Technologies®, Flowlogicv7.2). As a positive control the parental antibody was included. FIG. 6B shows the fold increase in mean fluorescence intensity for matured ADT1-4 clones compared to parental ADT1-4 binding primary skin derived human vδ1 cells. FIG. 6C shows the fold increase in mean fluorescence intensity for matured ADT1-7 clones compared to parental ADT1-7 binding the transformed PEER vδ1 cell line.

### Fold Improvements Over Parental Clones

### Dissociation-Enhanced Lanthanide Fluorescence Immunoassay (DELF/A)

For the confirmation of improvement in binding of affinity matures mAb to human and cyno antigens, DELFIA immunoassay was performed with the antigen directly coated to the plate (3 μg/mL of antigen in 50 μL PBS at 4° C. overnight (Nunc® #437111). For detection DELFIA Eu-N1 Anti-Human IgG (PerkinElmer® #1244-330) was used as secondary antibody at 1/500 dilution in 50 μL of 3% of MPBS (PBS+3% (w/V) skimmed milk powder). Development was with 50 μL of DELFIA enhancement solution (PerkinElmer® #4001-0010).

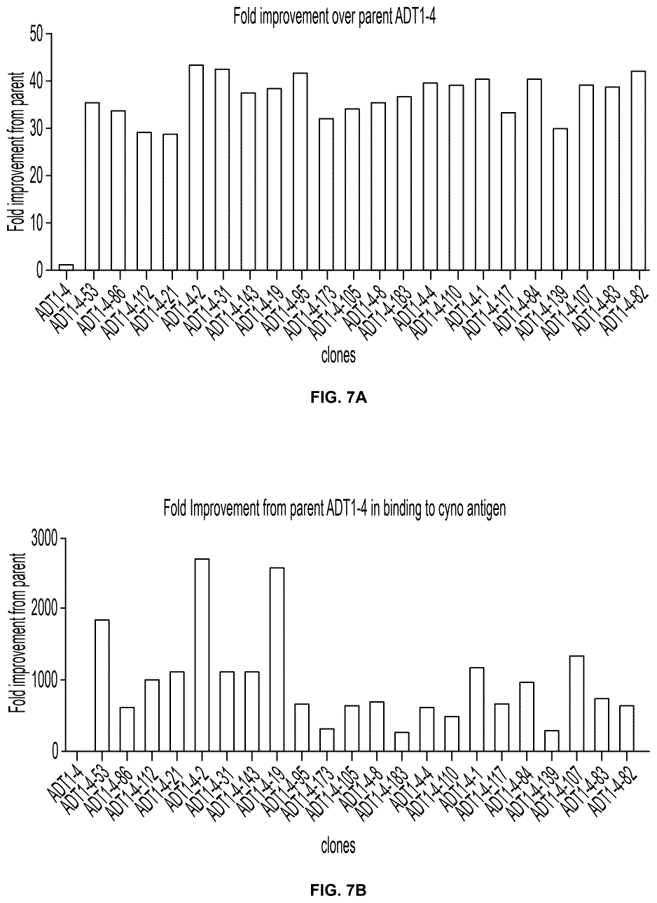

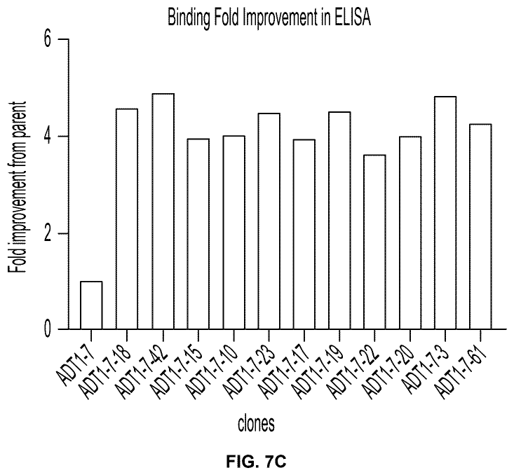

Affinity ranking of antibody of interest were performed using DELFIA immunoassay in which antibodies were captured via protein G coated on the plate and human soluble biotinylated L1 (DV1-GV4) antigen was added at 0.4 nM and cyno antigen DV1/GV77 at 10 nM (3MPBS). For detection 50 μL of streptavidin-Eu (1:500 in assay buffer, PerkinElmer®) was used and signal was developed with DELFIA enhancement solution. D1.3 hIgG1 (described in England et al. (1999) J. Immunol. 162: 2129-2136) was used as a negative control. The results are provided in FIGS. 7A to 7C.

### SPR Analysis—IgG Capture

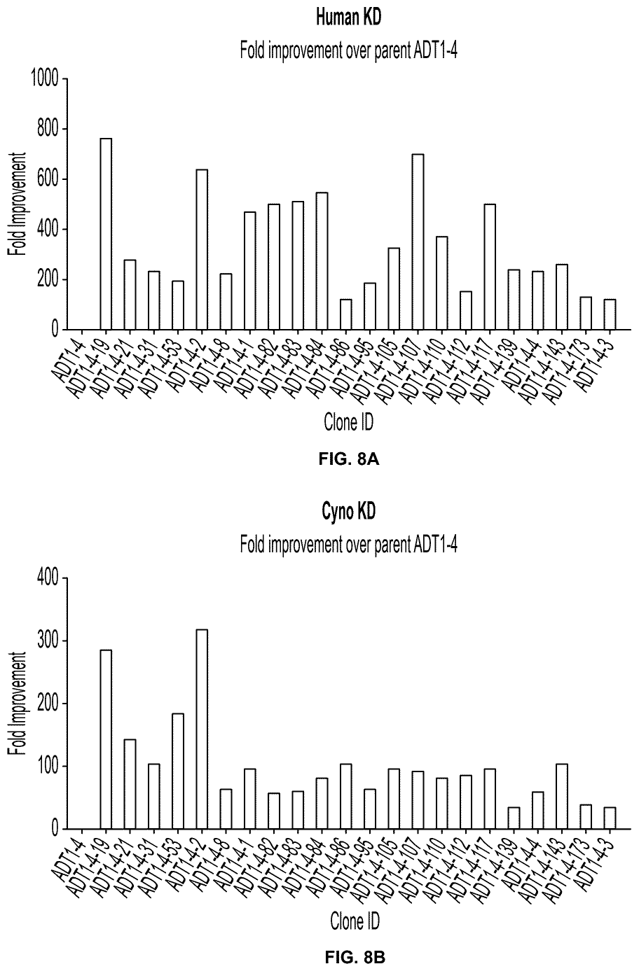

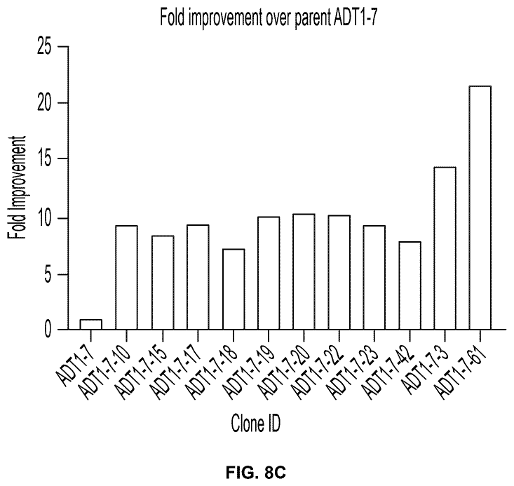

SPR analysis was used to compare KD values for the affinity matured clones compared to the parental clones. Instrument used: MASS-2 (Sierra® Sensors); Chip: amine high capacity (Sierra® Sensors); running buffer PBS+0.02% Tween 20 (Polysorbate 20). Experiments were performed at room temperature or 37° C., protein G was coupled to the chip. Antigen was flown over the cell in a dilution series ranging from 50 nM to 0.2 nM for human DV1-GV4 and from 100 nM to 1 nM for Cyno DV1-77. 120 sec association, 600 sec dissociation, 50 μL/min flowrate, regeneration with 10 mM glycine pH 1.5 kinetic fit according to Langmuir 1:1 binding using software Sierra® Analyzer. The results are shown in FIGS. 8A to 8C.

### Binding Affinity Analysis (KD by SPR)

### Vδ1 Monoclonal Antibody Binding Affinity to Vδ1 Antigen

The binding affinity of the antibodies to target (i.e. the Vδ1 chain of a γδ TCR) is established by SPR analysis using a Reichert® 4SPR instrument (Reichert Technologies®). Antibody (1.5 ug/mL) is coated onto a Planar Protein A Sensor Chip (Reichert Technologies®) to give an increase on baseline of approximately 500 uRIU. Antigen (e.g. L1 (DV1-GV4) was flown over the cell at a 1:3 dilution series from 300 nM to 3.7 nM with the following parameters: 180 s association, 600 s dissociation, flowrate 25 μL/min, running buffer PBS+0.05% Tween 20 (Polysorbate 20). All experiments were performed at room temperature. Steady state fitting was determined according to Langmuir 1:1 binding using software TraceDrawer (Reichert Technologies®).

### Human Antigen

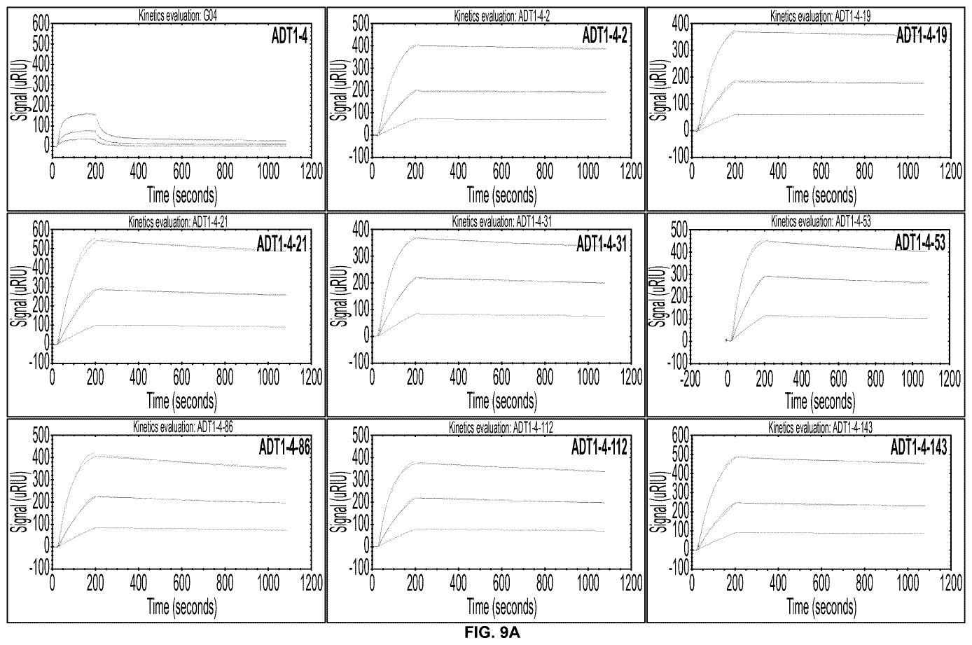

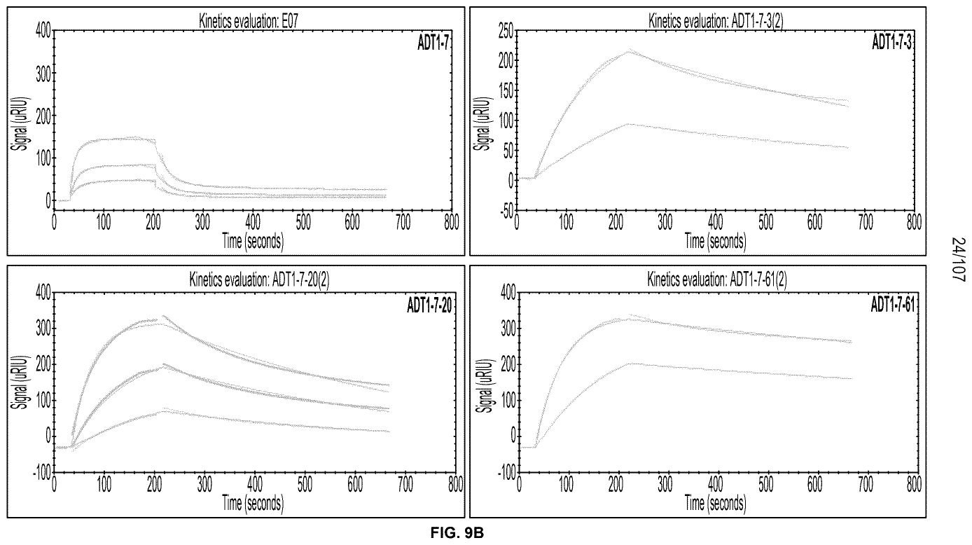

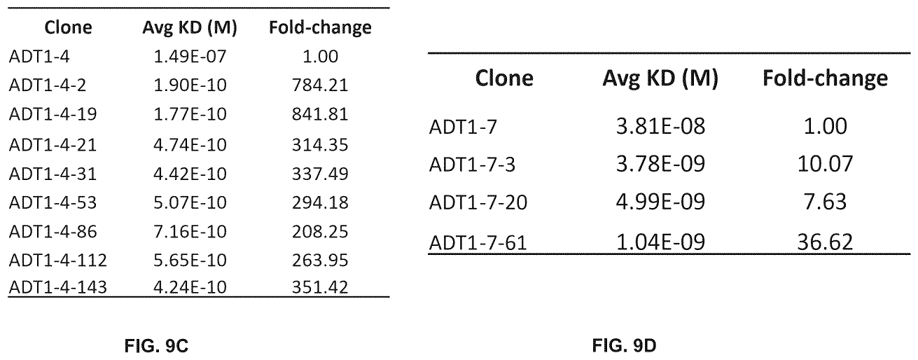

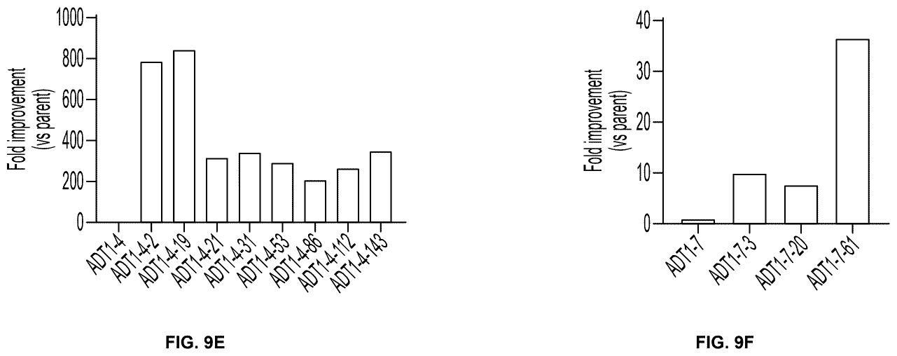

Affinity-matured Vδ1 mAbs exhibit greatly enhanced affinity to human Vδ1 antigen that either parent as determine by surface plasmon resonance (SPR) analysis (FIGS. 9A-9F). (FIGS. 9A, 9B) Surface Plasmon Resonance (SPR) analysis was performed with ADT1-4 (FIG. 9A) or ADT1-7 (FIG. 9B) parent mAbs and their affinity-matured derivatives to determine the binding affinity to human Vδ1 antigen. The binding interaction of Vδ1 and the affinity-matured mAbs were modelled according to Langmuir 1:1 binding. (FIGS. 9C, 9D) Tables summarizing the equilibrium dissociation constant (KD) of Vδ1-targeted antibodies, as derived from the sensograms illustrated in (FIG. 9A) and (FIG. 9B). The data is represented as the mean of two replicates performed on two different SPR instruments. (FIGS. 9E, 9F) Bar charts representing the fold increase in equilibrium dissociation constant (KD) of the affinity-matured derivative to Vδ1 antigen, as compared to their parent, ADT1-4 or ADT1-7.

Conclusions: This data demonstrates that the affinity-matured derivatives of ADT1-4 and ADT1-7 demonstrate significantly higher affinity to human Vδ1 antigen than the parent antibodies.

### Cyno Antigen

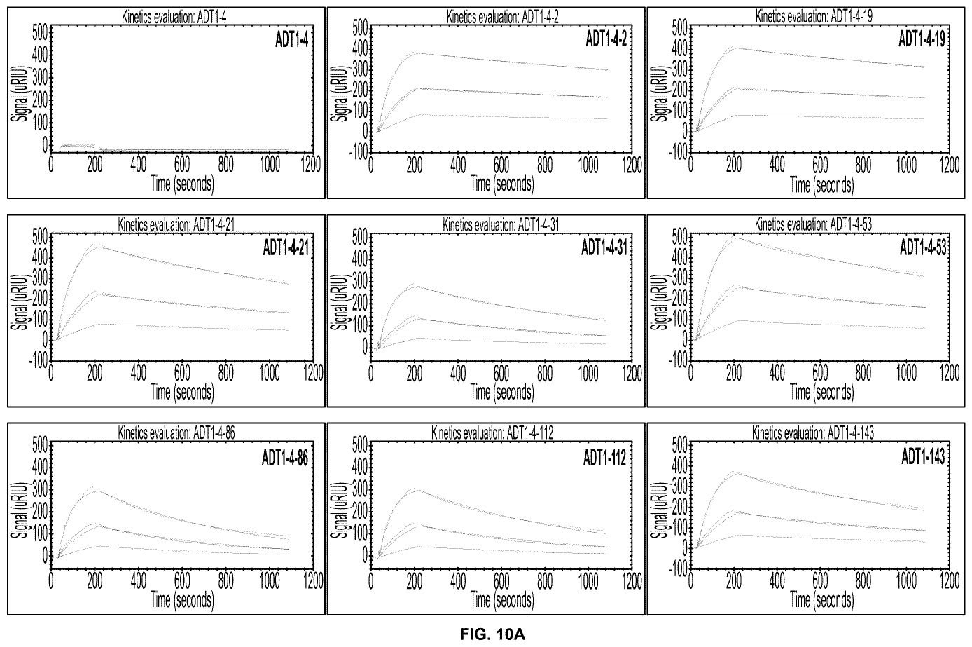

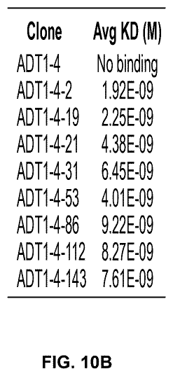

Surface plasmon resonance analysis demonstrates that the affinity-matured Vδ1 mAbs of the ADT1-4 lineage show a greatly enhanced affinity to cynomolgus Vδ1 antigen than the parent antibody (FIGS. 10A-10B). (FIG. 10A) Surface Plasmon Resonance (SPR) analysis was performed with ADT1-4 parent antibody and the affinity-matured derivatives to determine the binding affinity to cynomolgus Vδ1 antigen. The binding interaction of Vδ1 and the affinity-matured mAbs were modelled according to Langmuir 1:1 binding. (FIG. 10B) Table summarizing the equilibrium dissociation constant (KD) of Vδ1-targeted antibodies, as derived from the sensograms illustrated in (FIG. 10A). The data is represented as the mean of two replicates performed on two different SPR instruments. The results are shown in FIGS. 10A and 10B.

Conclusions: This data demonstrates that the affinity-matured derivatives of ADT1-4 demonstrate significantly higher affinity to cynomolgus Vδ1 antigen than the parent antibody.

### Binding Affinity to Cell Surface Vδ1 TCR (EC50 for Binding to Cell Surface Vδ1)

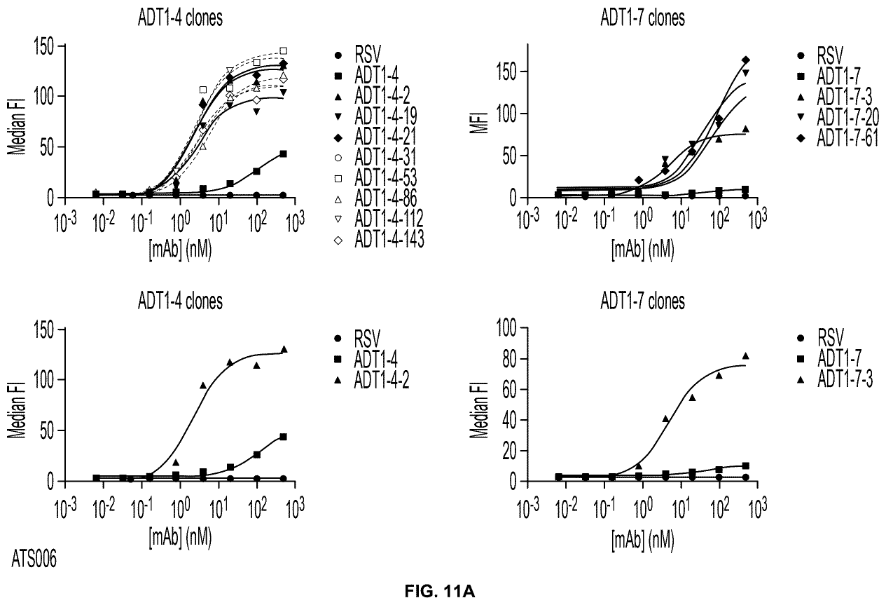

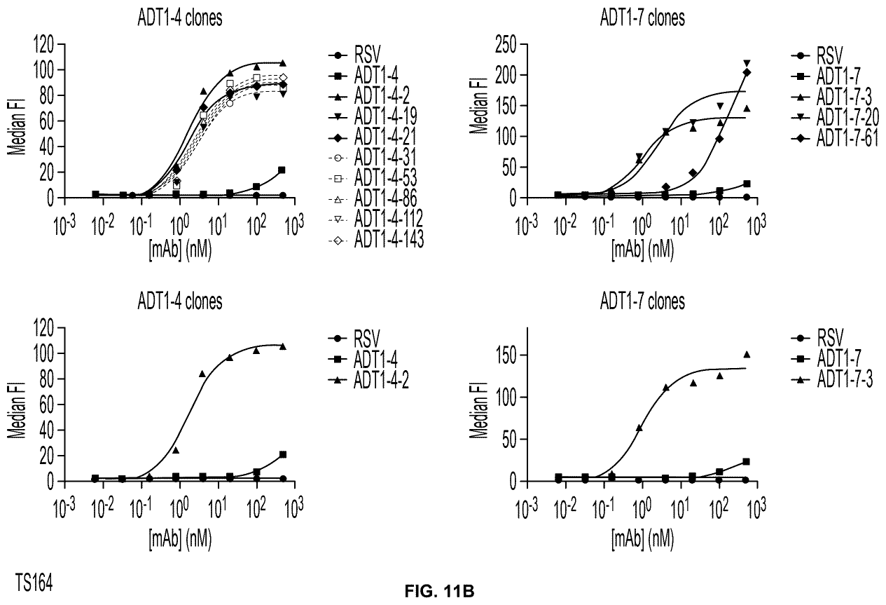

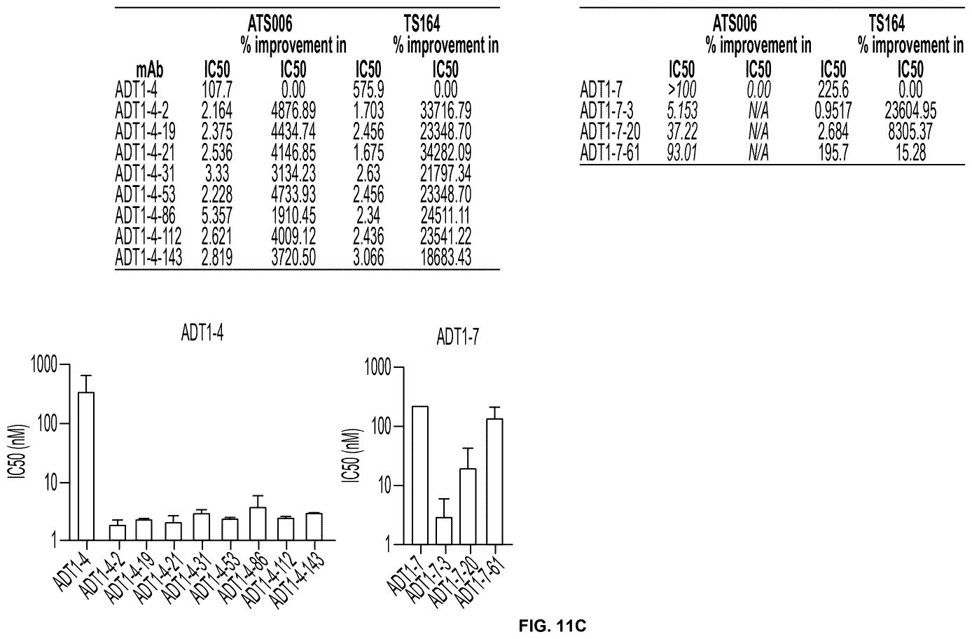

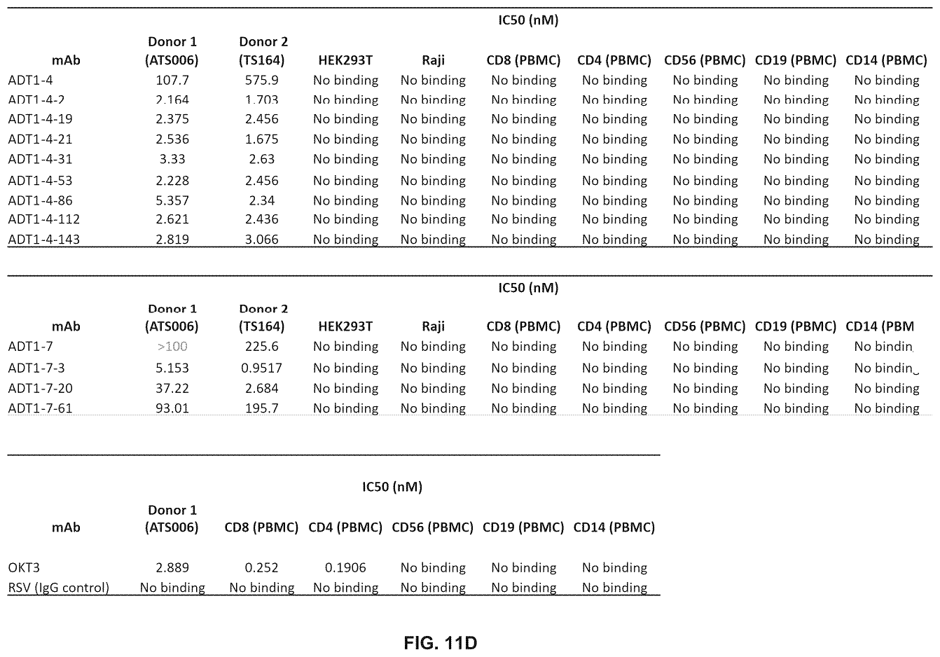

Affinity matured Vδ1 mAbs exhibit greatly enhanced affinity to Vδ1-positive γδT cells, with no demonstrable binding to cells lacking Vδ1, as shown in FIGS. 11A to 11D. (FIGS. 11A, 11B) The level of binding by the Vδ1 mAbs to two γδ T cell donors, ATS006 (FIG. 11A) and TS164 (FIG. 11B). γδ T cells were stained with varying concentrations of Vδ1 mAbs, followed by a fluorescent anti-human IgG detection antibody (xxx). All incubation steps were performed at 4° C. and mAb binding was determined using flow cytometry to measure the median level of fluorescence. Logarithmic four parameter dose-response curves were fitted using GraphPad® Prism 9. (FIG. 11C) Bar chart representing the average EC50s from the ADT1-4 and ADT1-7 clones binding to Vδ1-positive γδ T cell, represented as the mean two donors. (FIG. 11D) Table summarizing the EC50s plotted in (FIG. 11A) & (FIG. 11B), and Vδ1-negative cell types including HEK293A, Raji cells and various leukocyte subsets with primary blood mononuclear cells. For Vδ1-positive γδ T cell, data is represented as the mean two donors.

Conclusions: This data demonstrates that the affinity-matured derivatives of ADT1-4 and ADT1-7 demonstrate significantly higher affinity to Vδ1-positive γδ T cells than their parent mAbs, whilst showing no binding to Vδ1-negative cells HEK293T, Raji, or CD8, CD4, NK, CD19 or monocytic cells within PBMCs.

### Target Cell Binding of Vδ1 Monoclonal Antibodies

A range of target cells were assayed to determine the specificity and affinity of Vδ1 mAb binding. This included expanded skin-derived Vδ1 γδ T-cells, HEK293T cells, Raji cells, and multiple leukocyte subsets within human primary blood mononuclear cells. Adherent or semi-adherent cells (skin-derived Vδ1 γδ T-cells, HEK293T) were detached from tissue culture flask and resuspended in PBS. Similarly, non-adherent cell types (Raji, PBMC), were harvested and resuspended in PBS. Cells were seeded at a final density of 100,000 cells per well in v-bottom 96-well plates. Cells underwent centrifugation and the cell pellets were resuspended in FcR blocking reagent according to the manufacturer's instructions and incubated for 20 minutes at 4 C prior to a further wash. Vδ1 mAbs, anti-RSV IgG control and anti-CD3 OKT3 were diluted to 500 nM in PBS and serially diluted 1:5 in PBS to 6.4 pM, and added to the cells, followed by a 20-minute incubation at 4 C. To determine the quantity of mAb bound to the cell surface, the cells were then stained with a murine anti-human IgG secondary antibody, conjugated to APC (product code, dilution: 1:100). For Vδ1 γδ T-cells, HEK293T and Raji, the cells were also stained solely with a viability dye. For PBMC, conjugated antibodies against CD4, CD8, CD56, CD11b and CD19 (all at 1:100) were also included, allowing the discrimination of ap subsets, NK cells, B cell and monocytic cells. Following 20-minute incubation at 4 C, the cells were washed twice, and fluorescence measured using the MACSQuant®. IC50s are shown in FIG. 11D.

### Summary of KD Values for ADT1-4 Lineage (Human and Cyno)

The table below provides the KD values for the 24 clones in the ADT1-4 lineage and the ADT1-4 parent clone (G04) (for binding to human TRDV1 and cyno TRDV1):

| | HUMAN | HUMAN | CYNO | CYNO |
|---|---|---|---|---|
| Clone ID | K_{D} [M] | K_{D} (nM) | K_{D} [M] | K_{D} (nM) |
| ADT1-4 | 1.26E−07 | 126 nM | N/A | N/A |
| ADT1-4-19 | 1.63E−10 | 0.163 nM | 3.40E−09 | 3.40 nM |
| ADT1-4-21 | 4.41E−10 | 0.441 nM | 6.79E−09 | 6.79 nM |
| ADT1-4-31 | 5.22E−10 | 0.522 nM | 9.43E−09 | 9.43 nM |
| ADT1-4-53 | 6.08E−10 | 0.608 nM | 5.34E−09 | 5.34 nM |
| ADT1-4-2 | 1.95E−10 | 0.195 nM | 3.08E−09 | 3.08 nM |
| ADT1-4-8 | 5.41E−10 | 0.541 nM | 1.51E−08 | 15.1 nM |
| ADT1-4-82 | 2.49E−10 | 0.249 nM | 1.67E−08 | 16.7 nM |
| ADT1-4-83 | 2.41E−10 | 0.241 nM | 1.58E−08 | 15.8 nM |
| ADT1-4-84 | 2.27E−10 | 0.227 nM | 1.16E−08 | 11.6 nM |
| ADT1-4-86 | 9.39E−10 | 0.939 nM | 9.49E−09 | 9.49 nM |
| ADT1-4-95 | 6.42E−10 | 0.642 nM | 1.50E−08 | 15.0 nM |
| ADT1-4-105 | 3.81E−10 | 0.381 nM | 1.01E−08 | 10.1 nM |
| ADT1-4-107 | 1.77E−10 | 0.177 nM | 1.05E−08 | 10.5 nM |
| ADT1-4-110 | 3.33E−10 | 0.333 nM | 1.19E−08 | 11.9 nM |
| ADT1-4-112 | 7.84E−10 | 0.784 nM | 1.11E−08 | 11.1 nM |
| ADT1-4-117 | 2.45E−10 | 0.245 nM | 1.01E−08 | 10.1 nM |
| ADT1-4-139 | 4.99E−10 | 0.499 nM | 2.69E−08 | 26.9 nM |
| ADT1-4-4 | 5.19E−10 | 0.519 nM | 1.62E−08 | 16.2 nM |
| ADT1-4-143 | 4.63E−10 | 0.463 nM | 9.39E−09 | 9.39 nM |
| ADT1-4-173 | 9.07E−10 | 0.907 nM | 2.40E−08 | 24.0 nM |
| ADT1-4-3 | 9.35E−10 | 0.935 nM | 2.85E−08 | 28.5 nM |
| ADT1-4-1 | 8.00E−11 | 0.08 nM | 4.1E−10 | 0.41 nM |
| ADT1-4-6 | 1.06E−9 | 1.06 nM | 1.01E−9 | 1.01 nM |
| ADT1-4-138 | 4.38E−8 | 43.8 nM | 3.47E−8 | 34.7 nM |

### Summary of KD Values for ADT1-7 Lineage (Human Only)

The table below provides the KID values for the 11 clones in the ADT1-7 lineage and the ADT1-7 parental clone (F07) (for binding to human TRDV1 only):

| Clone ID | K_{D} [M] | K_{D} (nM) |
|---|---|---|
| ADT1-7 | 1.24E−08 | 12.4 nM |
| ADT1-7-10 | 1.30E−09 | 1.30 nM |
| ADT1-7-15 | 1.42E−09 | 1.42 nM |
| ADT1-7-17 | 1.26E−09 | 1.26 nM |
| ADT1-7-18 | 1.61E−09 | 1.61 nM |
| ADT1-7-19 | 1.19E−09 | 1.19 nM |
| ADT1-7-20 | 1.17E−09 | 1.17 nM |
| ADT1-7-22 | 1.18E−09 | 1.18 nM |
| ADT1-7-23 | 1.29E−09 | 1.29 nM |
| ADT1-7-42 | 1.51E−09 | 1.51 nM |
| ADT1-7-3 | 8.31E−10 | 0.831 nM |
| ADT1-7-61 | 5.57E−10 | 0.557 nM |

### Summary of Pharmacological Data for the Preferred Members of the ADT1-4 Lineage

| | HUMAN | HUMAN | | CYNO | CYNO | | | | |
|---|---|---|---|---|---|---|---|---|---|
| Clone | K_D | K_D | | K_D | K_D | IC50 TCR DR | IC90 TCR DR | IC50 | IC90 |
| ID | [M] | (nM) | | [M] | (nM) | (no THP-1) | (noTHP-1) | Killing | Killing |
| ADT1- | 1.26E−07 | 126 | nM | N/A | N/A | N/A | N/A | 2.603E−08 | 7.048E−08 |
| 4 | | | | | | | | (26.03 nM) | |
| ADT1- | 1.63E−10 | 0.163 | nM | 3.40E−09 | 3.40 nM | 3.67E−10 | 1.583E−9 | 1.12E−09 | 8.38E−09 |
| 4-19 | | | | | | (0.367 nM) | (1.583 nM) | (1.12 nM) | (8.38 nM) |
| ADT1- | 4.41E−10 | 0.441 | nM | 6.79E−09 | 6.79 nM | 2.66E−10 | 8.92E−10 | 9.4E−10 | 5.88E−09 |
| 4-21 | | | | | | (0.266 nM) | (0.892 nM) | (0.94 mM) | (5.88 nM) |
| ADT1- | 5.22E−10 | 0.522 | nM | 9.43E−09 | 9.43 nM | 2.45E−10 | 1.091E−9 | 1.63E−09 | 1.658E−08 |
| 4-31 | | | | | | (0.245 nM) | (1.091 nM) | (1.63 nM) | (16.58 nM) |
| ADT1- | 6.08E−10 | 0.608 | nM | 5.34E−09 | 5.34 nM | 2.54E−10 | 6.98E−10 | 1.05E−09 | 4.23E−09 |
| 4-53 | | | | | | (0.254 nM) | (0.698 nM) | (1.05 nM) | (4.23 nM) |
| ADT1- | 1.95E−10 | 0.195 | nM | 3.08E−09 | 3.08 nM | 3.1E−10 | 8.34E−10 | 8.7E−10 | 3.12E−09 |
| 4-2 | | | | | | (0.31 nM) | (0.834 nM) | (0.87 nM) | (3.12 nM) |
| ADT1- | 9.39E−10 | 0.939 | nM | 9.49E−09 | 9.49 nM | 3.07E−10 | 1.001E−9 | 1.05E−09 | 4.3E−09 |
| 4-86 | | | | | | (0.307 nM) | (1.001 nM) | (1.05 nM) | (4.3 nM) |
| ADT1- | 7.84E−10 | 0.784 | nM | 1.11E−08 | 11.1 nM | 2.95E−10 | 1.093E−9 | 9E−10 | 3.8E−09 |
| 4-112 | | | | | | (0.295 nM) | (1.093 nM) | (0.9 nM) | (3.8 nM) |
| ADT1- | 4.63E−10 | 0.463 | nM | 9.39E−09 | 9.39 nM | 3.13E−10 | 8.53E−10 | 1.78E−09 | 1.42E−08 |

| 4-143 | | | | | (0.313 nM) | (0.853 nM) | (1.78 nM) | (14.2 nM) |
|---|---|---|---|---|---|---|---|---|
| ADT1- | 8.00E−11 | 0.08 | 4.1E−10 | 0.41 | 3.604E−10 | 6.702E−10 | 1.4E−10 | 2.8E−10 |
| 4-1 | | | | | (0.3604 nM) | (0.6702 nM) | (0.14 nM) | (0.28 nM) |
| ADT1- | 1.06E−9 | 1.06 | 1.01E−9 | 1.01 | 4.796E−10 | 1.911E−9 | 1.4E−10 | 2E−10 |
| 4-6 | | | | | (0.4796 nM) | (1.911 nM) | (0.14 nM) | (0.2 nM) |
| ADT1- | 4.38E−8 | 43.8 | 3.47E−8 | 34.7 | 8.384E−9 | 3.83E−8 | 3.8E−10 | 6.44E−09 |
| 4-138 | | | | | (8.384 nM) | (38.3 nM) | (0.38 nM) | (6.44 nM) |

### Summary of Pharmacological Data for the Preferred Members of the ADT1-7 Lineage

| Clone | K_D | K_D | | IC50 TCR DR | IC90 TCR DR | IC50 | IC90 |
|---|---|---|---|---|---|---|---|
| ID | [M] | (nM) | | (no THP-1) | (noTHP-1) | Killing | Killing |
| ADT1-7 | 1.24E−08 | 12.4 | nM | — | — | 5.53E−09 | 7.48E−08 |
| | | | | | | (5.53 nM) | (74.8 nM) |
| ADT1-7-20 | 1.17E−09 | 1.17 | nM | 1.64E−10 | 1.036E−9 | 2.80E−10 | 2.47E−09 |
| | | | | (0.164 nM) | (1.036 nM) | (0.28 nM) | (2.47 nM) |
| ADT1-7-3 | 8.31E−10 | 0.831 | nM | 1.61E−10 | 7.90E−10 | 1.60E−10 | 1.06E−09 |
| | | | | (0.161 nM) | (0.79 nM) | (0.16 nM) | (1.06 nM) |
| ADT1-7-61 | 5.57E−10 | 0.557 | nM | 6.264E−9 | 1.704E−6* | 4.20E−10 | 1.66E−08 |
| | | | | (6.264 nM) | (1704.445 nM) | (0.42 nM) | (16.6 nM) |

*Artefact of data due to 100% inhibition not being reached for this clone

### Example 5. Functional Characterisation

### Vδ1 Monoclonal Antibodies on TCR Downregulation

The capacity of ADT mAb to engage with and resultantly downregulate γδ T-cell receptors is evaluated by measuring TCR expression. Skin γδ T-cells (from donor ATS006 and TS164) were seeded into a 96 well round bottom plate at 3×10\hat{ }5 cells/mi in γδ media with increasing concentration of the test mAbs (range from 0.00067 to 67 nM) or the highest concentration (67 nM) of the corresponding isotype control (hIgG1, RSV), diluted in PBS. The cells were incubated for 2 hours in a humidified 002 chamber at 37° C. The cells were washed and stained for dead cells (Thermo Fisher Scientific®#15580607) and their VD1 TCRs (Miltenyi Biotec® #130-117-697) for 30 minutes at 4° C. The cells were washed in FACS buffer and resuspended in Cell Fix (BD® sciences #340181) before incubating overnight at 4° C. in the dark. The VD1 TCR expression level, determined by median fluorescence intensity (MFI), was measured by flow cytometry the following day using the MACSQuant® Analyzer 16.

The results are shown in FIGS. 12A-12G, which shows affinity matured Vδ1 mAbs bind and downregulate Vδ1 TCR more effectively than their parental clone and OKT3. (FIGS. 12A and 12B). Quantification of γδ TCR expression after 2 hours incubation of γδ cells with Vδ1 mAbs and OKT3 before staining of the Vδ1 TCR with a-Vδ1 TCR-PE-Vio770. MFI of a-Vδ1 staining was acquired using flow cytometry and calculated from the TCR positive gate before calculating % MFI from the MFI of TCR positive, untreated γδ cells. The upper graph represents data from the ADT1-4 lineage and the lower graph represents data from the ADT1-7 lineage. (FIG. 12C) Bar chart representing the IC50 values calculated from (FIG. 12A). (FIGS. 12D and 12E). Bar chart representing the IC50 values in (FIG. 12B) presented as fold improvement from the parent ADT1-4 (left) and ADT1-7 (right). (FIG. 12F) Table representing IC50 values from (FIG. 12B) with the percentage improvement calculated from the respective parents of ADT1-4 (upper) and ADT1-7 (lower). (FIG. 12G) Table representing IC50 values from (FIG. 12B) with the fold improvement calculated from the respective parents of ADT1-4 (upper) and ADT1-7 (lower).

### Vδ1 Monoclonal Antibodies on γδ Activation Measured by CD107a Expression

The capacity of ADT mAb to engage with and activate VD1 γδ cells is evaluated by measuring CD107a expression. Skin γδ T-cells (from donor ATS006) were seeded into a 96 well round bottom plate at 6×10\hat{ }5 cells/ml in γδ media with THP-1 cells (ATCC®-TIB-202) at 1.2×10\hat{ }6 cells/ml and increasing concentration of the test mAbs (range from 0.00067 to 67 nM) or the highest concentration (67 nM) of the corresponding isotype control (hIgG1, RSV), diluted in PBS. The cells were incubated for 2 hours in a humidified CO2 chamber at 37° C. The cells were washed and stained for dead cells (Thermo Fisher Scientific®#15580607), VD1 TCRs (Miltenyi Biotec® #130-117-697) and aCD107a (Miltenyi Biotec® #130-112-610) for 30 minutes at 4° C. The cells were washed in FACS buffer before incubating overnight at 4° C. in the dark. The VD1 TCR and CD107a expression level, determined by median fluorescence intensity (MFI), was measured by flow cytometry the following day using the MACSQuant® Analyzer 16.

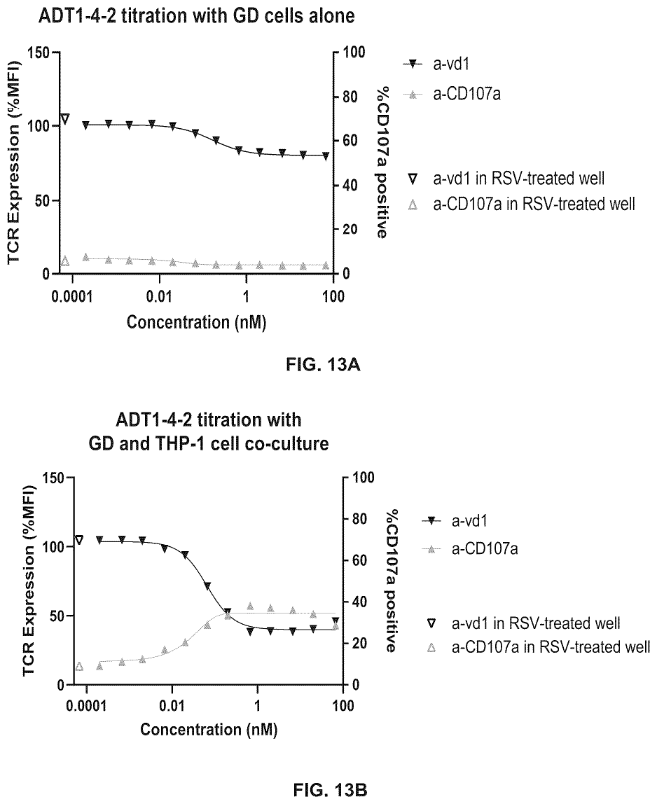

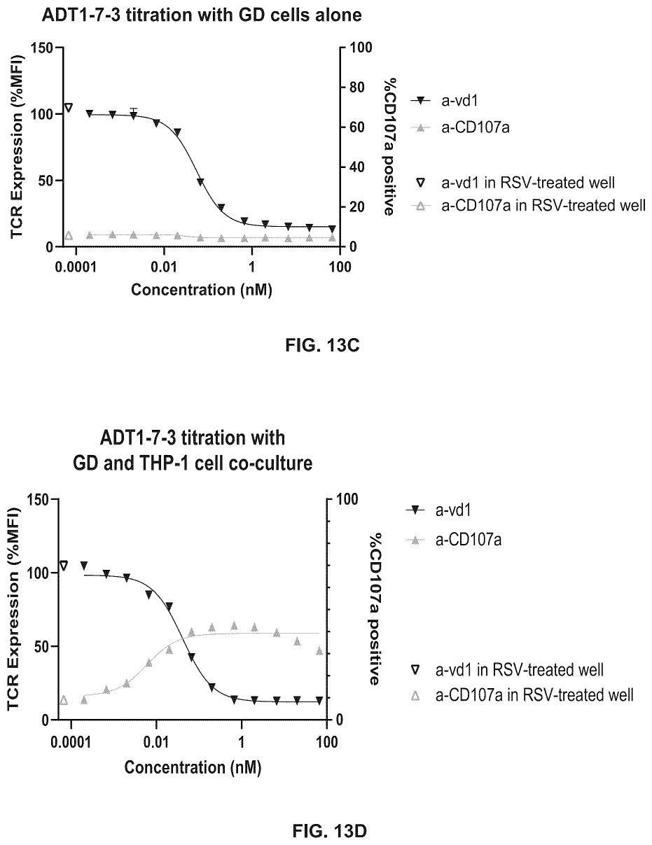

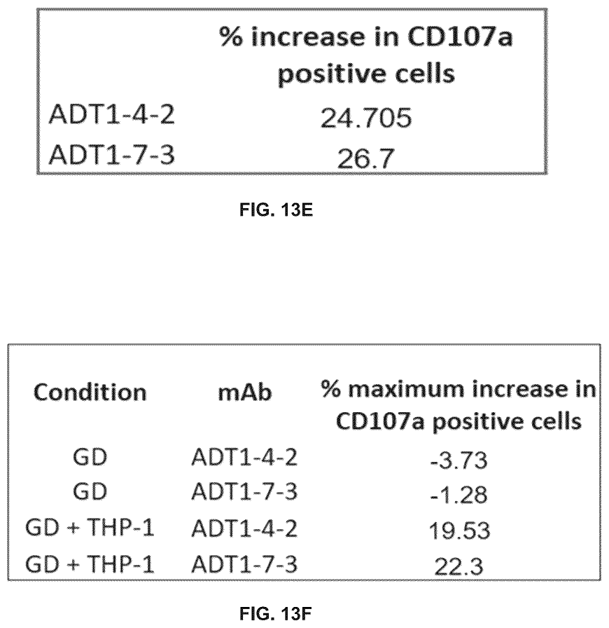

The results are shown in FIGS. 13A-13F, which shows affinity matured Vδ1 mAbs induce activation only when in the presence of target cells. (FIGS. 13A and 13B) Quantification of γδ TCR expression (black) and CD107a expression (grey) after 2 hours incubation of γδ cells with ADT1-4-2 alone (upper) and with THP-1 cells (lower) at a 1:2 target-to-effector ratio before staining of the Vδ1 TCR and CD107a with a-Vδ1 TCR-PE-Vio770 and a-CD107a-VioBlue respectively. MFI of Vδ1 staining was acquired using flow cytometry and calculated from the TCR positive gate before calculating % MFI from the MFI of TCR positive, untreated and non-cocultured γδ cells. MFI of CD107a staining was acquired using flow cytometry and calculated from the ‘NOT THP-1’ gate before calculating % MFI from the CD107a MFI of untreated non-cocultured γδ cells. (FIGS. 13C and 13D) Quantification of γδ TCR expression (black) and CD107a expression (grey) after 2 hours incubation of γδ cells with ADT1-7-3 alone (upper) and with THP-1 cells (lower) at a 1:2 target-to-effector ratio before staining of the Vδ1 TCR and CD107a with a-Vδ1 TCR-PE-Vio770 and a-CD107a-VioBlue respectively. MFI of Vδ1 staining was acquired using flow cytometry and calculated from the TCR positive gate before calculating % MFI from the MFI of TCR positive, untreated and non-cocultured γδ cells. MFI of CD107a staining was acquired using flow cytometry and calculated from the ‘NOT THP-1’ gate before calculating % MFI from the CD107a MFI of untreated non-cocultured γδ cells. (FIG. 13E) Table representing the percentage increase in γδ CD107a expression from cocultured cells treated with the highest concentration of Vδ1 mAb compared to untreated non-cocultured γδ cells. (FIG. 13F) Table representing the percentage increase in γδ CD107a expression from cocultured cells treated with the highest concentration of Vδ1 mAb compared to untreated, co- and non-cocultured γδ cells.

### Vδ1 Monoclonal Antibodies on γδ Activation Measured by CD25 Expression

The capacity of ADT mAb to engage with and activate VD1 γδ cells is evaluated by measuring CD25 expression. Skin γδ T-cells (from donor ATS006) were seeded into a 96 well round bottom plate at 6×10\hat{ }5 cells/ml in γδ media with THP-1 cells (ATCC®-TIB-202) at 1.2×10\hat{ }6 cells/ml and increasing concentration of the test mAbs (range from 0.00067 to 67 nM) or the highest concentration (67 nM) of the corresponding isotype control (hIgG1, RSV), diluted in PBS. The cells were incubated for 24 hours in a humidified CO2 chamber at 37° C. The cells were washed and stained for dead cells (Thermo Fisher Scientific®#15580607), VD1 TCRs (Miltenyi Biotec® #130-117-697) and CD25 (Miltenyi Biotec® #130-113-280) for 30 minutes at 4° C. The cells were washed in FACS buffer before incubating overnight at 4° C. in the dark. The VD1 TCR and CD107a expression level, determined by median fluorescence intensity (MFI), was measured by flow cytometry the following day using the MACSQuant® Analyzer 16.

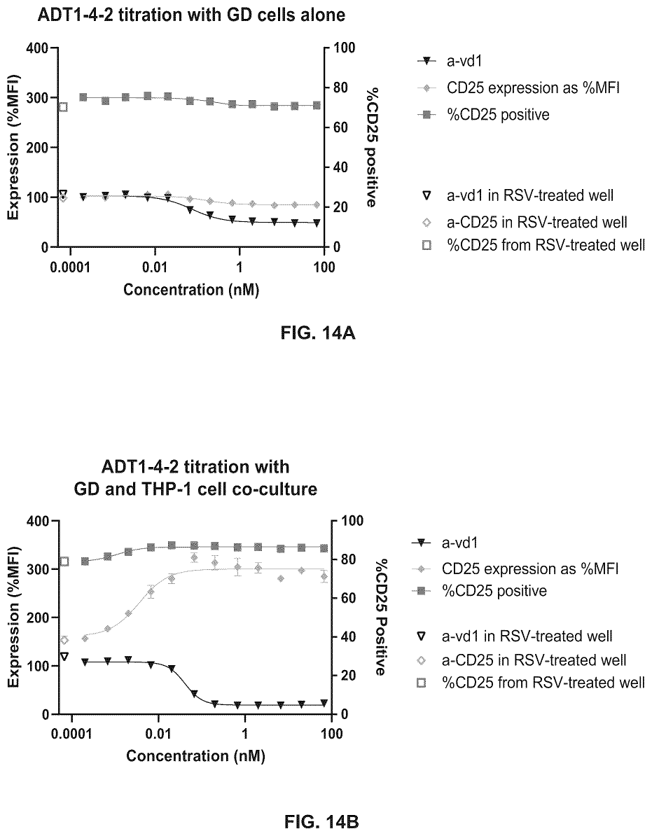

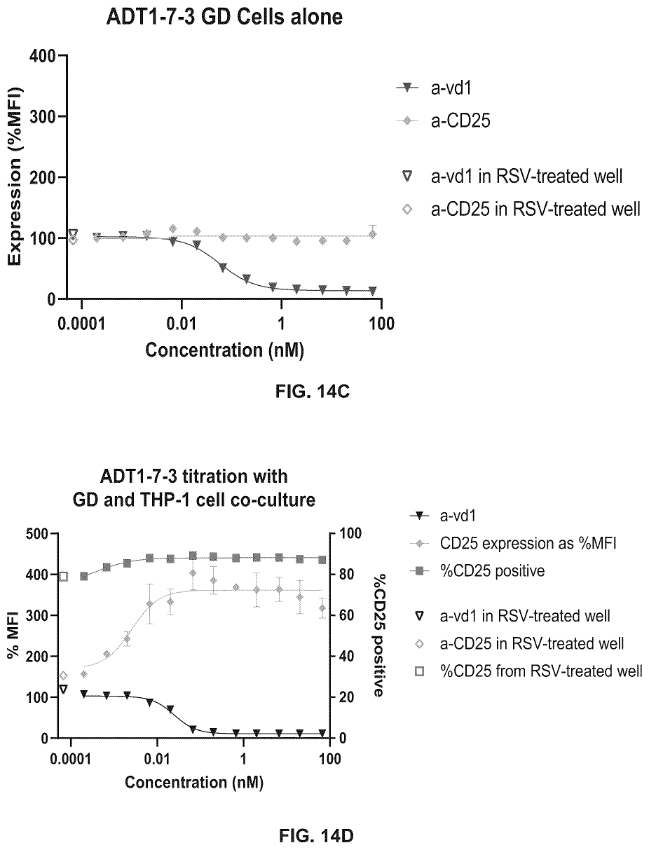

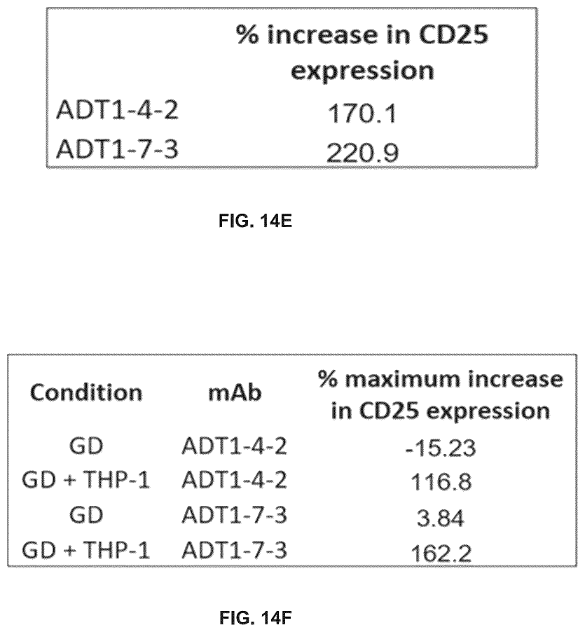

The results are shown in FIGS. 14A-14F, which shows affinity matured Vδ1 mAbs induce activation only when in the presence of target cells. (FIGS. 14A and 14B) Quantification of γδ TCR expression (black) and CD25 expression (grey) after 24 hours incubation of γδ cells with RSV (added at 67 nM) and ADT1-4-2 alone (upper) and with THP-1 cells (lower) at a 1:2 target-to-effector ratio before staining of the Vδ1 TCR and CD25 with a-Vδ1 TCR-PE-Vio770 and a-CD25-VioBlue respectively. MFI of Vδ1 staining was acquired using flow cytometry and calculated from the TCR positive gate before calculating % MFI from the MFI of TCR positive, untreated and non-cocultured γδ cells. MFI of CD25 staining was acquired using flow cytometry and calculated from the ‘NOT THP-1’ gate before calculating % MFI from the CD25 MFI of untreated non-cocultured γδ cells. (FIGS. 14C and 14D) Quantification of γδ TCR expression (black) and CD25 expression (grey) after 2 hours incubation of γδ cells with RSV (added at 67 nM) and ADT1-7-3 alone (upper) and with THP-1 cells (lower) at a 1:2 target-to-effector ratio before staining of the Vδ1 TCR and CD25 with a-Vδ1 TCR-PE-Vio770 and a-CD25-VioBlue respectively. MFI of Vδ1 staining was acquired using flow cytometry and calculated from the TCR positive gate before calculating % MFI from the MFI of TCR positive, untreated and non-cocultured γδ cells. MFI of CD25 staining was acquired using flow cytometry and calculated from the ‘NOT THP-1’ gate before calculating % MFI from the CD25 MFI of untreated non-cocultured γδ cells. (FIG. 14E) Table representing the percentage increase in γδ CD25 expression from cocultured cells treated with the highest concentration of Vδ1 mAb compared to untreated non-cocultured γδ cells. (FIG. 14F) Table representing the percentage increase in γδ CD25 expression from cocultured cells treated with the highest concentration of Vδ1 mAb compared to untreated, co- and non-cocultured γδ cells.

### Cyno TCR Downregulation

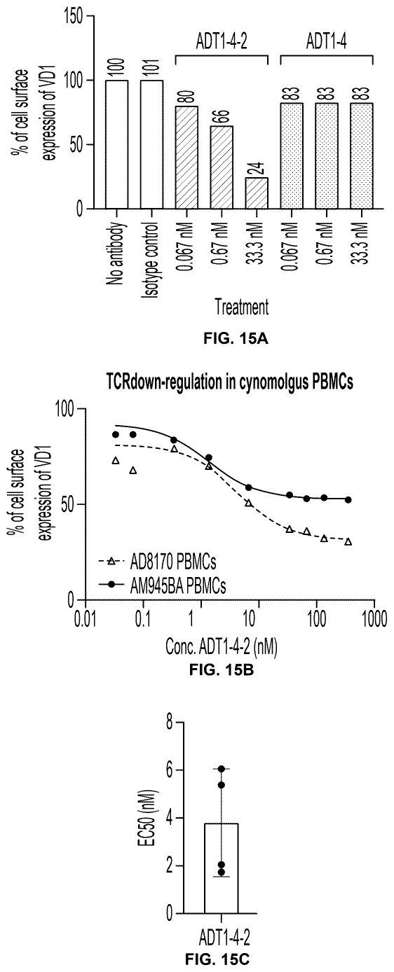

Studies were undertaken to explore the capability of ADT1-4-2 to bind to cynomolgus vδ1 gamma delta T cells and to induce its TCR down-regulation. For these studies, cynomolgus monkeys PBMCs were collected from fresh blood and put into culture for 14 days to expand γδ-T cells. Alternatively, αβ-T cells were depleted from the total PBMCs population using magnetic beads coated with an anti-αβ antibody (Clone R73) before to be expanded in vitro. Expansion was as followed, 250,000 cells were added per well of a U-bottom 96-well plate in RPMI-1640 media containing 10% of FCS, antibiotics (P/S) and a cocktail of cytokines (IFNγ, IL21, IL4, IL1 and IL15). Seven days later, cells were spiked with 10 μl of fresh media containing 10% of FCS, antibiotics (P/S) and the cytokines IL21 and IL15. It was followed by the replacement of 100 μl of media by fresh media containing 10% of FCS, antibiotics (P/S) and IL15 at day 11. At day 14, cells were collected and subjected to the TCR down-regulation assay. This assay consists of mixing the cells with various concentrations of antibodies for 2 hours. After the incubation time, cells were stained for flow cytometry analysis for the following markers: CD3, TCR-αβ, TCR-γδ and VD1. A live/death cell dye was also included to discriminate the live population of cells. Flow cytometry analysis was performed by gating on the CD3+, TCR-γδ+ and VD1+ cell population and VD1 mean florescence intensity was measured inside the VD1+ gate. Isotype control (anti-RSV) or no antibodies were used as negative controls at the highest concentration only. Data were normalized to the control without any antibody. FIG. 15A shows the comparison of ADT1-4-2 and ADT1-4 capability to engage and reduce the expression of VD1 on cynomolgus γδ-T cells. Results shows a dose response reduction of VD1 expression upon treatment with ADT1-4-2 (up to 76% at 33.3 nM) but not ADT1-4 antibody. In that particular assay, γδ-T cells were expanded from an αβ-T cell depleted population. A 500-time fold range of concentration was used. FIG. 15B shows the percentage of cell surface expression of VD1 upon treatment with ADT1-4-2. In this assay, expansion of γδ-T cells was from the all PBMCs population. A 10,000-fold range of concentration was used. Data shows a dose response reduction of VD1 cellular expression in 2 different donors (AD8170 and AM945BA) as an example. A total of 4 donors was tested and EC50 was extracted individually using GraphPad Prism nonlinear fit curve function. FIG. 15C shows the individual EC50 value for the different donors combined with the mean and standard deviation. The mean is 3.83±2.23 nM.

### Antibodies:

CD3-AF700 (BD Bioscience®, clone SP34-2, ref:557917)
TCR-αβ-AF647 (BioLegend®, clone R73, ref: 201116)
TCR-γδ-BV421 (BioLegend®, clone B1, ref: 331218
VD1-PE (eBioscience®, clone TS8.2, ref: 12-5679-42)

### Enhanced Cytotoxicity of Affinity Matured Clones

Affinity maturation of both ADT1-4 and ADT1-7 Vδ1 clones significantly enhances the cytotoxic effect of Vδ1 γδ T-cells in the in vitro THP-1 killing assay

### THP-1 Cell Killing Assay

Vδ1 mAbs and anti-RSV IgG control were diluted to 1 μg/ml in PBS and serially diluted 1:10 in PBS before adding to 384-well ultra imaging assay plates (PerkinElmer®). THP-1 cells (ATCC®) cultured in RPMI; 10% FCS (Invitrogen®) were stained with [0.5 μM] CellTrace™ CFSE live cell dye for 20 minutes. Expanded skin derived Vδ1 γδ T-cells were detached from tissue culture flasks and re-suspended in basal growth media before mixing 1:1 with THP-1 cells in suspension. Cell suspensions were seeding into 384-well assay plates to give a final cell seeding density of 1,000 THP-1 cells per well and 2,000 Vδ1 γδ T-cells per well. mAbs were diluted in the final assay volumes to concentrations ranging from 200 ng/ml to 0.2 pg/ml. THP-1 and Vδ1 γδ T-cells were cultured in the presence of Vδ1 mAbs at 37° C., 5% CO2 for 24 hours. To determine the numbers of live THP-1 cells after 24 hours, confocal images were acquired using an Opera Phenix high content platform capturing nine fields of view at 10× magnification. Live cell counts were quantified base on size, morphology, texture and intensity of live cell stains.

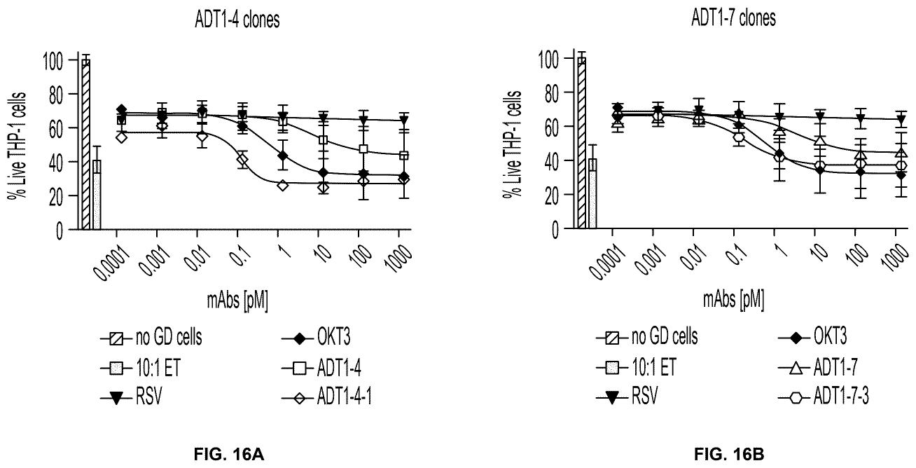

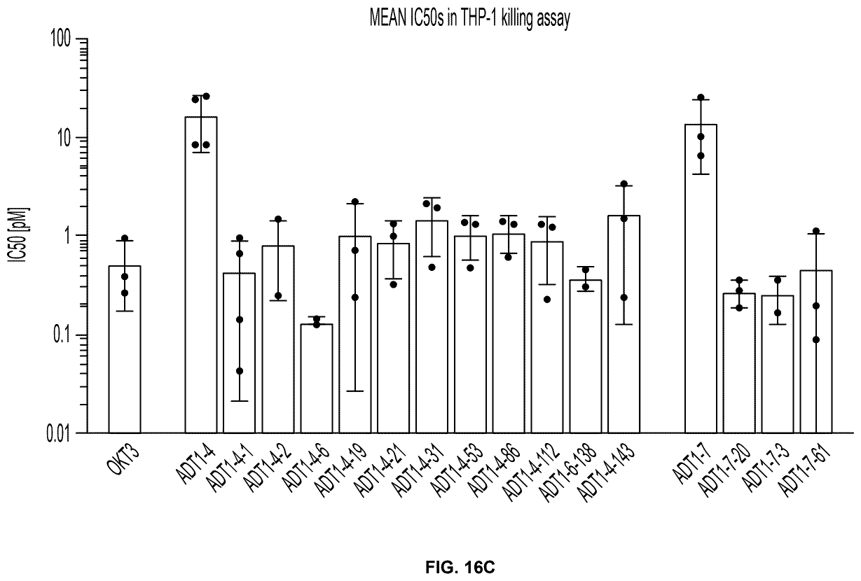

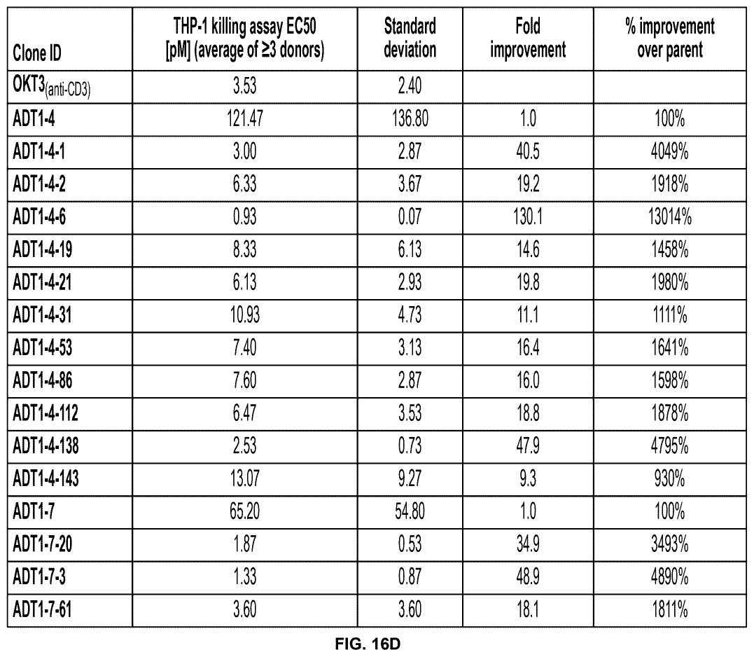

FIGS. 16A-16D show the affinity matured Vδ1 mAbs exhibit enhanced potency in a THP-1 high content cytotoxicity assay compared to parental clones. (FIGS. 16A & 16B) Quantification of live THP-1 cell numbers after 24 hours co-culture with γδ T-cells in the presence of Vδ1 mAbs or controls [Effector:Target ratio 2:1]. Cell numbers were normalized as a percentage of the no γδ T-cell control (100%). OKT3 (anti-CD3 antibody) and an anti-RSV isotype control were included as assay controls. Cell numbers were calculated using high content confocal microscopy on live cells. Graphs are representative of parental ADT1-4 (FIG. 16A) and ADT1-7 (FIG. 16B) clones. (FIG. 16C) Bar chart representing the average EC50s from OKT3 control, and the ADT1-4 and ADT1-7 clones. Data is represented as Mean±standard deviation of n=3 biological replicates. (FIG. 16D) Table summarizing the EC50s plotted in (FIG. 16C). Data is represented as Mean, Standard deviation, fold change and percentage improvement compared to ADT1-4 and ADT1-7 parental clones.

### Vδ1 Monoclonal Antibodies on Antibody-Dependent Cellular Cytotoxicity (ADCC)

Expanded skin-derived Vδ1 γδ T-cells from three donors were detached from tissue culture flasks and re-suspended in basal growth media and seeded at a final density of 20,000 cells per well in white 96-well plates. Raji cells were similarly seeded at 20,000 per well, to be used as a positive control for ADCC induced by the CD20-targeted antibody Rituximab. mAbs were diluted and added to each well in final concentrations ranging from 1 to 100 nM. FcγRIIIa+ ADD Bioassay effector cells were thawed, and 62,500 cells were added to each well followed by incubation at 37° C., 5% CO2 for 4.5 hours. Luciferin-containing Bio-Glo reagent was added to each well, followed by a further 10-minute incubation, and luminescence measured using a plate reader.

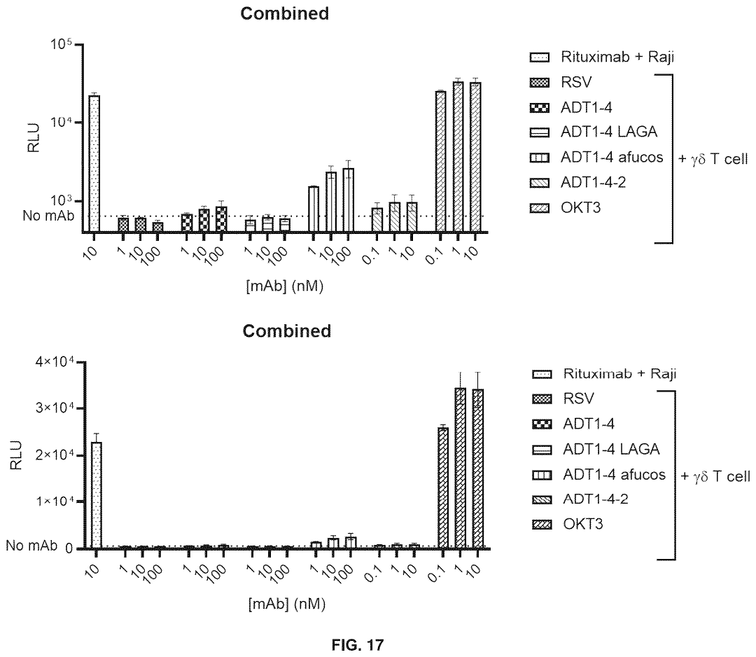

FIG. 17 shows the Vδ1-targeted mAbs induce negligible antibody-dependent cellular cytotoxicity (ADCC). ADCC, as measured using a surrogate reporter assay in which downstream NFAT signalling is measured by induction of luciferase activity within FcγRIIIa+ effector cells. The effector cells were co-cultured with three γδ T-cell donors (Effector:Target ratio 3) and varying concentrations of monoclonal antibody. CD20⁺ Raji cells and Rituximab were included as a positive control. Bioluminescence was measured after 4.5 hours of co-culture. Data is represented as mean±SD of three biological replicates (three γδ T-cell donors).

Conclusions: This data demonstrates that ADT1-4 parent and the affinity matured derivative ADT1-4-2 does not induce ADCC, demonstrated via a FcγRIIIa+ effector reporter cell line in which NFAT signalling induces luciferase activity. OKT3, however, appears to induce high levels of ADCC-specific signalling.

### Vδ1 Monoclonal Antibodies on Complement-Dependent Cytotoxicity (CDC)

Expanded skin-derived Vδ1 γδ T-cells were detached from tissue culture flasks and re-suspended in basal growth media and seeded at a final density of 75,000 cells per well in white 96-well plates. Raji cells were similarly seeded at 75,000 per well, to be used as a positive control for CDC induced by the CD20-targeted antibody Rituximab. mAbs were diluted and added to each well in final concentrations ranging from 1 to 100 nM. Human serum, either fresh or heat-inactivated, was added to a final concentration of 40%. Heat-inactivation was performed by warming the serum in a water-bath at 56 C for 30 minutes, followed by cooling on ice. The cultures were then incubated at 37° C., 5% CO2 for 24 hours. To determine the numbers of live cells after 24 hours, the cells were harvested and stained with viability dye at a 1:1000 dilution for 20 minutes. Fluorescence was measured using the MACSQuant® and live cell counts were quantified based on negative staining for the viability dye.

The results are shown in FIG. 18, which demonstrates Vδ1-targeted mAbs do not induce complement-dependent cytotoxicity (CDC). The number of viable γδ T cells following culture with 40% human serum and 50 nM anti-CD3 OKT3 or anti-Vδ1 ADT1-4-2. and varying concentrations of monoclonal antibody. Serum was used fresh or underwent prior heat-inactivation. CD20⁺ Raji cells and Rituximab were included as a positive control. Viability was measured after 24 hours of co-culture via viability dye. Data is represented as mean±SD of three biological replicates.

Conclusions: This data demonstrates that ADT1-4-2 does not induce complement-dependent cytotoxicity (CDC), with no increase in cytotoxicity of Vδ1-positive cells in the presence of complement-containing serum. Rituximab, however, shows strong CDC stimulation against CD20-positive Raji cells.

### Healthy Cell Sparing

Studies were undertaken to explore the effect of stimulating/activating vδ1 cells with anti-vδ1 antibody with respect to cytotoxicity towards healthy cells. This was tested by incubating the anti-vδ1 clone ADT1-4-2 with PBMC and then assessing monocyte cytotoxicity and apoptosis.

Cryopreserved human peripheral blood mononuclear cells (PBMC) were commercially sourced from 3 healthy donors. PBMC were seeded into round bottom 96-well tissue culture plates at 250,000 cells/well in 250 ul of complete media (RPMI supplemented with 10% FCS, pen/strep, non-essential amino acids, sodium pyruvate and HEPES) with 10 ng/ml IL15. A titration vδ1 antibody ADT1-4-2 was added to a final concentration of 6.6 nM. RSV IgG, IgG2a and OKT3 antibodies were included as controls. Stimulations were performed for 20 hours. Flow cytometry analysis was performed at the endpoint to determine the monocyte phenotype in each condition and apoptotic monocytes. on vδ1 cells. Cells were gated firstly on live singlets, followed by CD14 (Miltenyi Biotec® 130-110-523) to identify monocytes. Apoptotic monocytes were then identified through positive ApoTracker™ Green staining (Biolegend® 427402).

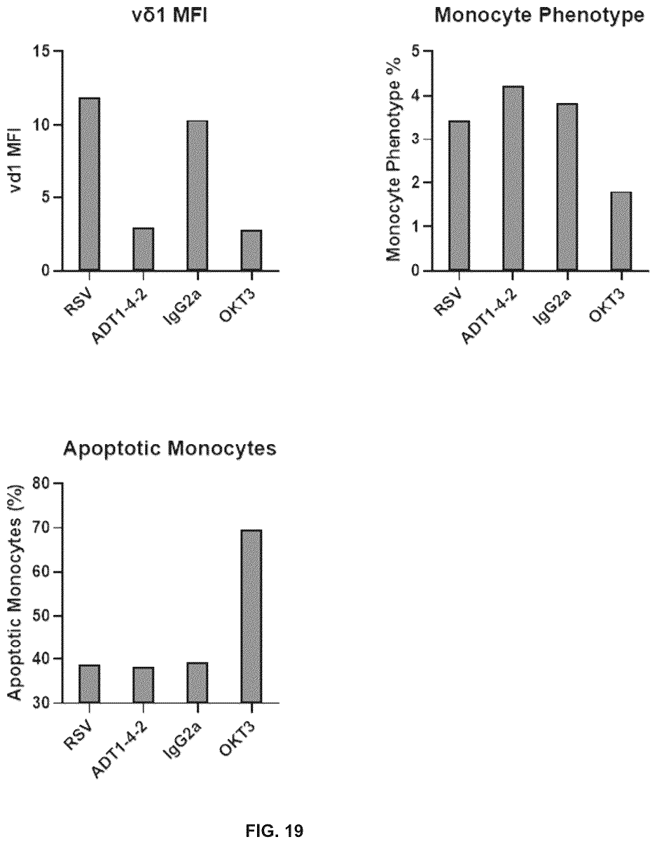

FIG. 19 (upper left panel) shows the vδ1 TCR staining MFI upon antibody stimulation as an indication of target engagement. FIG. 19 (upper right panel) and (lower panel) show the endpoint monocyte phenotype and apoptotic monocytes (respectively) following antibody stimulation. Stimulation with the vδ1 antibody clone ADT1-4-2 engaged Vδ1 cells but did not cause healthy monocyte cytotoxicity.

### Example 6. Effect of Anti-V81 Antibody on Til Populations from Primary Tumour Biopsies

### Anti-Vδ1 Antibody Promoted Activation, Proliferation, and Cytotoxicity in Tumour-Infiltrating Lymphocytes.

Studies were undertaken to explore anti-Vδ1 antibody-conferred modulation of human tumour-infiltrating lymphocytes (TILs). For these studies, human renal cell carcinoma (RCC) tumour biopsies were shipped fresh and processed upon receipt. Biopsies were cut into pieces measuring ˜2 mm² and TILs were obtained using an adaptation of the method originally described by Kupper and Clarke (Clarke et al, 2006). Specifically, up to four 2 mm² biopsies were placed on 9 mm×9 mm×1.5 mm Cellfoam matrices, and one matrix was placed per well on a 24-well plate. Biopsies were then cultured in 2 ml Iscove's Modified Dulbecco's Medium (IMDM) supplemented with 4% human plasma, β-mercaptoethanol (50 μM), penicillin (100 U/ml), streptomycin (100 μg/ml), amphotericin B (2.5 μg/ml), HEPES (10 mM), Na Pyruvate (1 mM), MEM Non-Essential Amino Acids Solution (1×) and IL-15 (2 ng/ml, Miltenyi Biotec®). 1 ml of medium was aspirated every 3-4 days and replaced with 1 ml complete medium containing 2× concentrated IL-15. TILs were harvested 10 or 11 days later, passed through a 70 μM nylon cell strainer, centrifuged at 300×g for 5 minutes and resuspended in complete medium for counting. 400,000 cells were then plated per well in 96-well plates prior to stimulation with anti-Vδ1 antibodies. TILs were stimulated with ADT1-4, ADT1-4-2, ADT1-7-3, or RSV IgG1 isotype control antibodies in the presence of IL-15 at a concentration of 2 or 50 ng/ml.

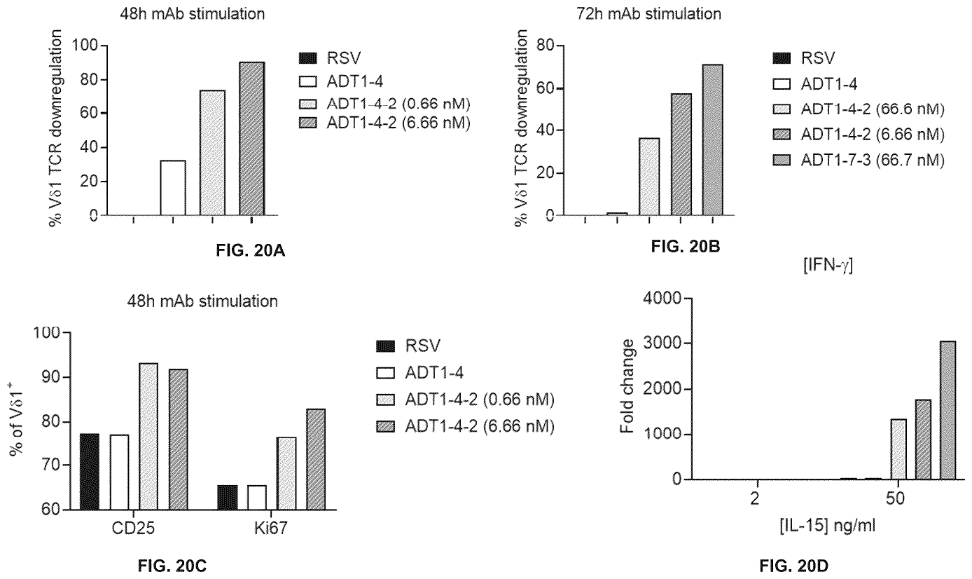

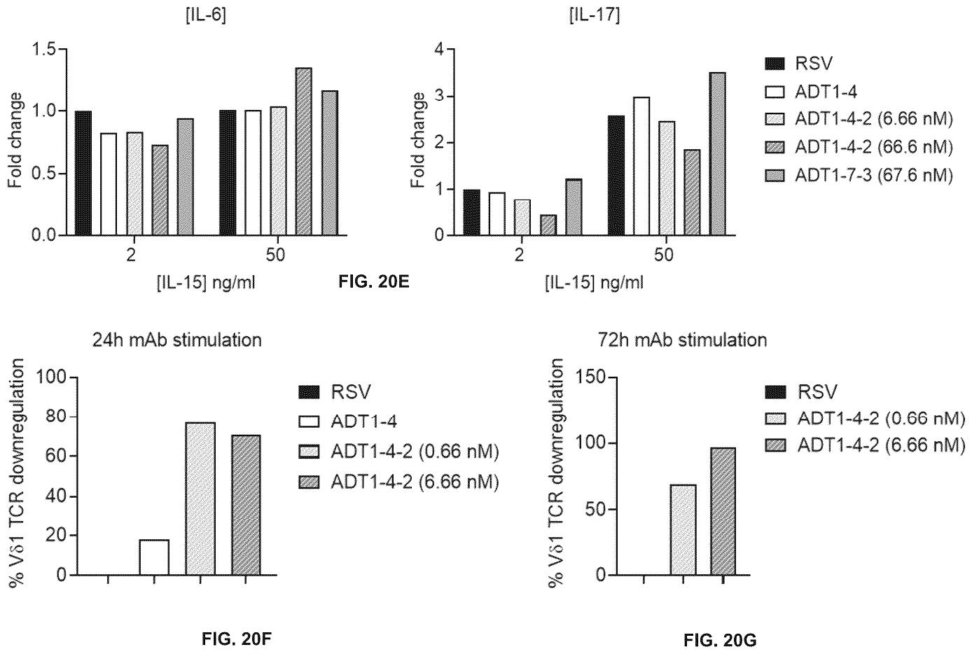

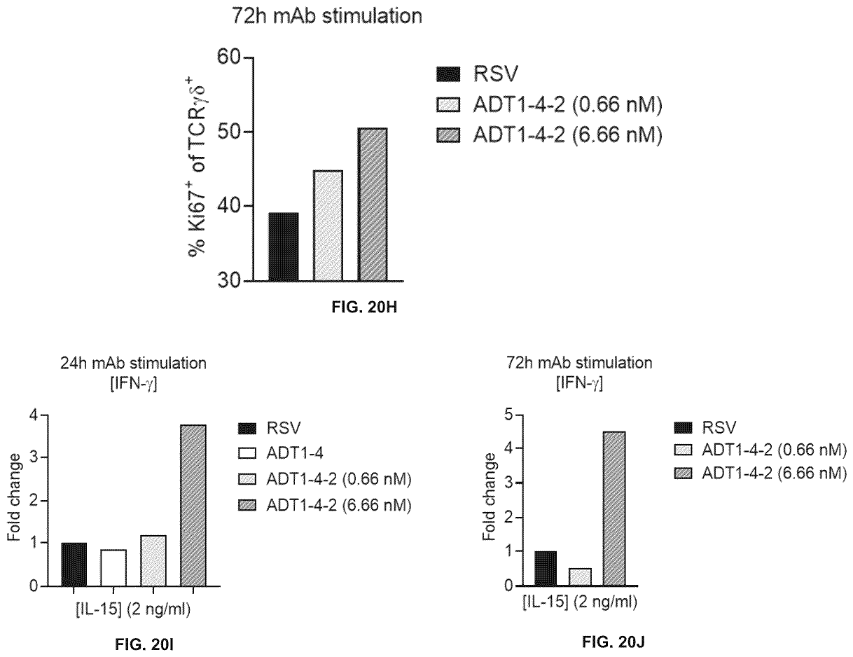

FIGS. 20A and 20B show the % decrease in Vδ1 TCR expression on total tumour infiltrating-γδ T cells following 48 (FIG. 20A) or 72 (FIG. 20B) hours mAb stimulation in two separate donors, confirming target engagement in each case. Tumour-infiltrating Vδ1⁺ cells were analysed for expression of CD25 and Ki-67. FIG. 20C shows enhanced expression of both CD25 and Ki-67 on Vδ1⁺ T cells following 48-hour stimulation with ADT1-4-2 compared with stimulation with IgG1 isotype control or ADT1-4. Supernatants from cultures of mAb-stimulated TILs were analysed for cytokine production by MSD. FIG. 20D shows substantial fold increases in IFN-γ production by TILs stimulated with ADT1-4-2 or ADT1-7-3 for 72 hours in the presence of 50 ng/ml IL-15. FIG. 20E shows that stimulation of TILs with ADT-1-4-2 or ADT1-7-3 did not enhance secretion of type 17-associated cytokines IL-6 or IL-17 at this timepoint.

Alternatively, biopsies from different donors were digested enzymatically upon receipt to obtain a single cell suspension. Specifically, up to 1 g of tissue was placed into a Miltenyi Biotec® C tube along with 4.7 ml RPMI supplemented with enzymes from Miltenyi Biotec's® Tumour Dissociation Kit at concentrations recommended by the manufacturer aside from Enzyme R which was used at 0.2× concentration to prevent cleavage of pertinent cell surface molecules. C-Tubes were placed on the gentleMACS™ Octo Dissociator with heating blocks attached. Program 37C_h_TDK_1 for the dissociation of small tumours was selected. After 1 hour the digest was filtered through a 70 μM filter and complete IMDM containing 4% human plasma was added to quench enzymatic activity. Cells were then washed twice and resuspended in complete IMDM for counting. Depending on cell numbers, 2×10⁶ or 4×10⁶ cells were plated per well in 48-well plates and were stimulated with ADT1-4, ADT1-4-2, or RSV IgG1 isotype control antibodies in the presence of IL-15 at a concentration of 2 ng/ml. TILs isolated by enzymatic digestion were analysed by flow cytometry 24-72 hours post mAb stimulation. FIGS. 20F and 20G show the % decrease in Vδ1 TCR expression on total tumour infiltrating-γδ T cells following mAb stimulation at 24 (FIG. 20F) or 72 (FIG. 20G) hours in two individual donors, confirming target engagement in TILs isolated by enzymatic digestion. FIG. 20H shows dose-dependent enhanced expression of Ki-67 on γδ T cells following 72 hours stimulation with ADT1-4-2. Supernatants from cultures of mAb-stimulated TILs were analysed for cytokine production by MSD. FIGS. 20I and 20J show the fold increase in IFN-γ produced by TILs isolated from two individual donors by enzymatic digestion and stimulated with ADT1-4-2 at a concentration of 6.66 nM in the presence of 2 ng/ml IL-15 at two time points, 24 hours (FIG. 20I) and 72 hours (FIG. 20J). In summary, these combined results highlight the ability of affinity matured anti-Vδ1 antibody to drive activation, proliferation and anti-tumorigenic cytokine production by tumour-infiltrating γδ T cells within 72 hours of stimulation.

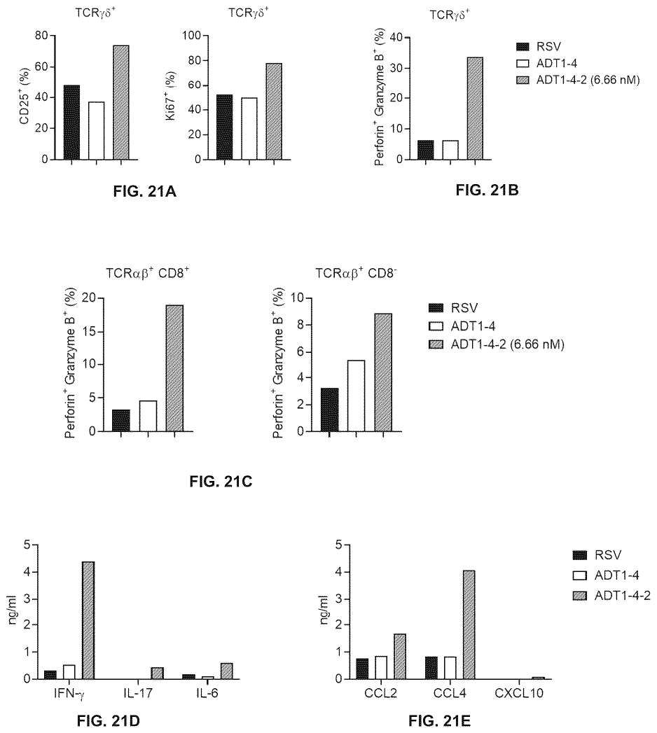

In another experiment, lung tumour-derived TILs were isolated on grid matrices and stimulated with ADT1-4, ADT1-4-2 or IgG1 isotype control for 10 days in the presence of 2 ng/ml IL-15. FIG. 21A shows enhanced expression of CD25 and Ki-67 in γδ T cells stimulated for 10 days with ADT1-4-2. FIG. 21B shows a substantial increase in Perforin⁺ Granzyme B⁺ γδ T cells following 10 days stimulation with ADT1-4-2. Stimulation with mAbs over a longer time period allowed for the analysis of immune licensing of αβ T cells via anti-Vδ1 antibody-stimulated Vδ1⁺ T cells. FIG. 21C shows considerably enhanced expression of Granzyme B and Perforin by both CD8⁺ and CD8⁻ αβ T cells following 10 days stimulation of tumour-infiltrating Vδ1⁺ T cells with ADT1-4-2. Supernatants from TILs cultured for 10 days with mAbs were analysed by MSD. FIG. 21D shows markedly increased production of IFN-γ, and moderately increased production of IL-17 and IL-6 by lung tumour-derived TILs following stimulation with ADT1-4-2. FIG. 21E demonstrates enhanced production of the chemokines CCL2, CCL4 and CXCL10 by TILs following 10 days stimulation with ADT1-4-2. In summary, these results highlight the ability of anti-Vδ1 antibody to drive activation and cytotoxicity in tumour-infiltrating γδ T cells, which can then promote cytotoxicity in tumour-infiltrating αβ T cells.

### Example 7. Vδ1-PD-L1 Bispecific Antibody Binding Affinity to Human PD-L1 Antigen

The binding affinity of the antibodies to recombinant human PD-L1 is established by SPR analysis using a Reichert 4SPR instrument (Reichert Technologies®). Antibody (1.5 ug/mL) is coated onto a Planar Protein A Sensor Chip (Reichert Technologies®) to give an increase on baseline of approximately 500 uRIU. Recombinant human PD-L1 was flown over the cell at a concentration of 50 nM, 25 nM and 12.5 nM with the following parameters: 180 s association, 480 s dissociation, flowrate 25 μL/min, running buffer PBS+0.05% Tween 20 (Polysorbate 20). Experiments were performed at 4° C. The results are shown in FIGS. 29A-29F. It is shown that the binding affinity to recombinant human PD-L1 antigen is similar for each of the 20 clones tested (which utilise 10 different PD-L1 binding Fcab regions).

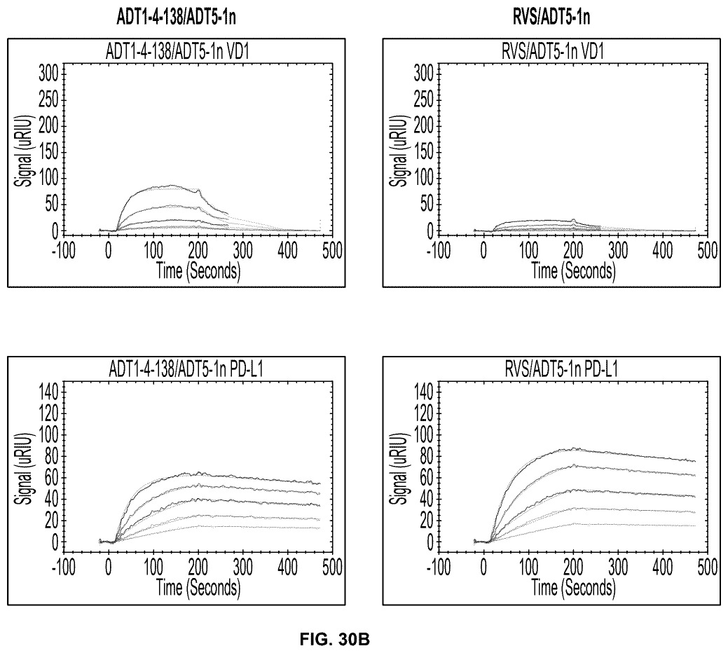

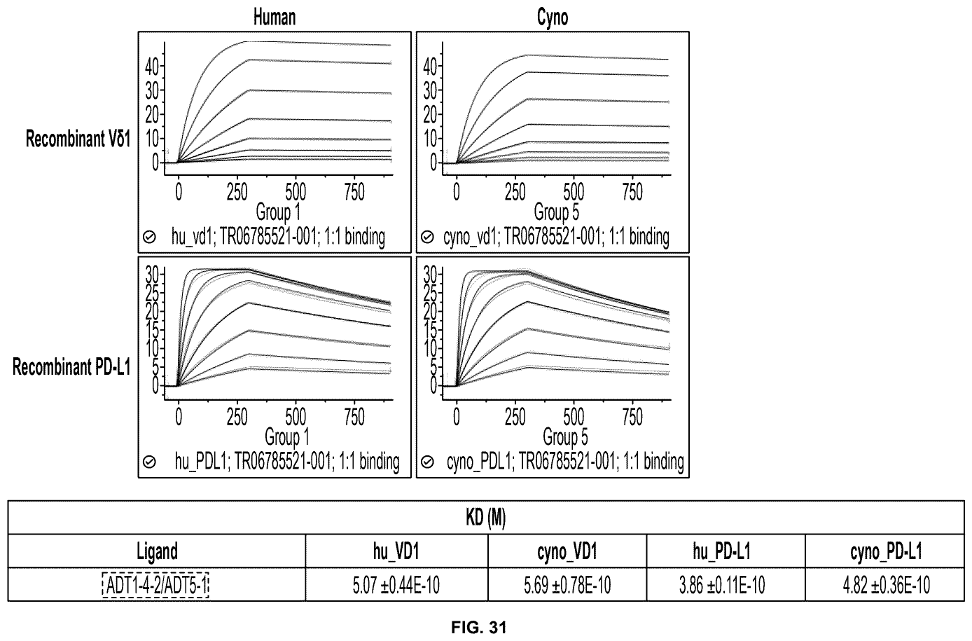

It is also shown that dialling up and down the affinity of the anti-Vδ1 portion of the Vδ1-PD-L1 bispecific antibodies does not significantly affect the affinity for binding to human PD-L1. In this regard, FIGS. 30A-30B show the affinity for binding to human PD-L1 when the same PD-L1 binding Fcab is paired with a “high Vδ1 affinity”, a “mid Vδ1 affinity” and a “low Vδ1 affinity” Fab. Specifically, the binding affinity of the antibodies to recombinant human PD-L1 established by SPR analysis using a Reichert® 4SPR instrument (Reichert Technologies®). Antibody (0.5 ug/mL) is coated onto a Planar Protein A Sensor Chip (Reichert Technologies®) to give an increase on baseline of approximately 500 uRIU. Recombinant human PD-L1 was flown over the cell at a concentration of 100 nM, 50 nM, 25 nM, 12.5 nM, 6.75 nM with the following parameters: 180 s association, 480 s dissociation, flowrate 25 μL/min, running buffer PBS+0.05% Tween 20 (Polysorbate 20). Experiments were performed at 4° C. The results are shown in FIGS. 30A-30B.

Conclusions: This data demonstrates that the Vδ1/PD-L1 bispecific antibodies demonstrate binding to human PD-L1 that is comparable to that of the monospecific control anti-PD-L1 antibodies. Furthermore, the different mutations in the Fcab region do not affect the affinity to recombinant human PD-L1. In addition, altering the anti-Vδ1 Fab of the Vδ1/PD-L1 bispecific antibodies does not affect the affinity for binding to human PD-L1.

### Example 8. Vδ1-PD-L1 Bispecific Antibody Binding Affinity to Human Vδ1 Human PD-L1 Antigen

The binding affinity of the antibodies to recombinant human and cynomolgous Vδ1 and PD-L1 was established by SPR analysis using a Biacore™ 8K+(Cytiva®) with a Carboxymethylated dextran coated CM5 chip. To assess binding affinity to Vδ1, an anti-human IgG antibody was amine coupled to the CM5 chip. To assess binding affinity to PD-L1, an anti-human Fab antibody was amine coupled to the CM5 chip. Antibodies were then captured at 1 μg/ml, with the following parameters: 30 s, 10 uL/min flow rate to achieve a baseline of 50-80 RU capture level, before flowing human or cyno recombinant Vδ1 or PD-L1 over the chips at 50 nM, 25 nM, 12.5 nM, 6.25 nM, 3.13 nM, 1.56 nM, 0.78 nM 0.39 nM, with the following parameters: 300 s association at 30 uL/min flow rate, 600 s 30 uL/min dissociation rate, and 60 s 30 uL/min regeneration. This is an alternative method of SPR than was used in Example 7, but the same Vδ1/PD-L1 bispecific antibody clone was used in both examples. The results are shown in FIG. 31.

Conclusions: This data demonstrates that Vδ1/PD-L1 bispecific antibody demonstrates equipotent binding to human and cyno Vδ1 and PD-L1.

### Example 9. Target Cell Binding of Vδ1-PD-L1 Bispecific Antibodies

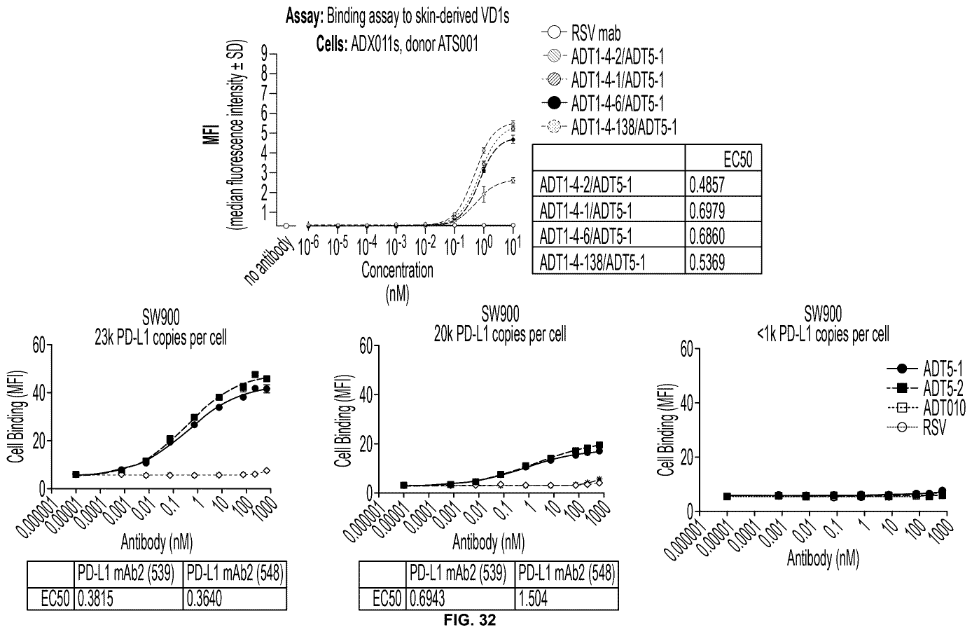

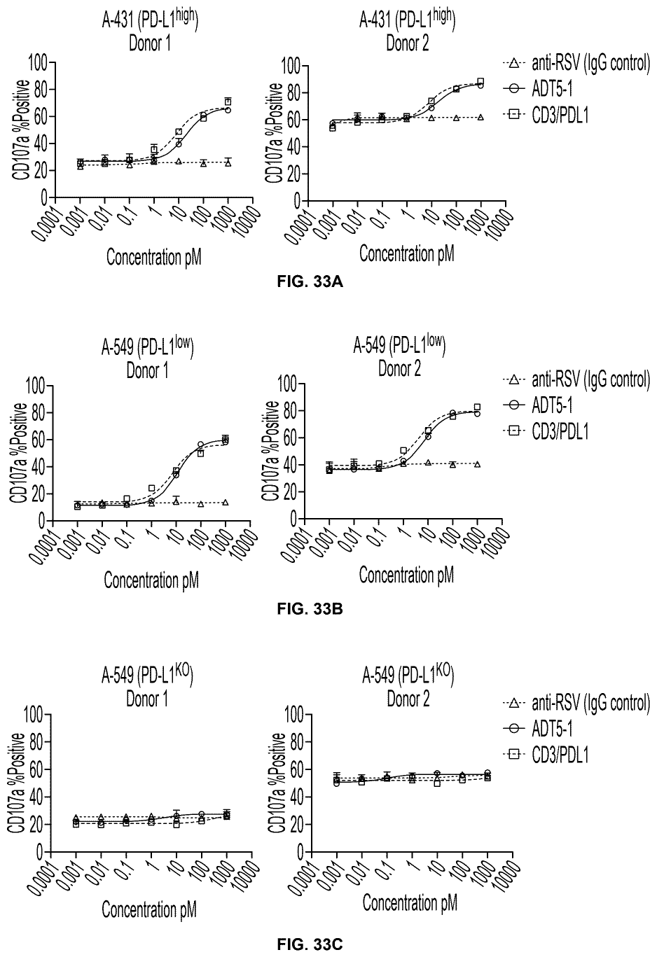

Skin derived Vδ1 γδT cells (ADX011 skin-Vδ1s) and PD-L1 positive tumor cells (SW900, MDA-MB-231) and Vδ1−/PD-L1− negative control cells (HEK293) were assayed to determine the specificity and on cell binding affinity of PD-L1/Vδ1 bispecific antibodies. Cells were detached from tissue culture flask, resuspended in PBS and seeded at a final density of 100,000 cells per well in v-bottom 96-well plates. Cells underwent centrifugation and the cell pellets were resuspended in FcR blocking reagent according to the manufacturer's instructions and incubated for 20 minutes at 4° C. prior to a further wash. Antibodies were diluted to 500 nM in PBS and serially diluted 1:10 in PBS to 50 pM, and added to the cells, followed by a 20-minute incubation at 4 C. To determine the quantity of mAb bound to the cell surface, the cells were then stained with a murine anti-human IgG secondary antibody, conjugated to APC (dilution: 1:100) in addition to a viability dye. Following 20-minute incubation at 4 C, the cells were washed twice, and fluorescence measured using the MACSQuant®. The results are shown in FIG. 32.

Conclusions: This data demonstrates that the Vδ1/PD-L1 bispecific antibodies demonstrate binding to Vδ1-positive γδT-cells and to tumor cells in a PD-L1 dependent manner.

### Example 10. Assessing γδ T Cell Activation and Degranulation In Vitro

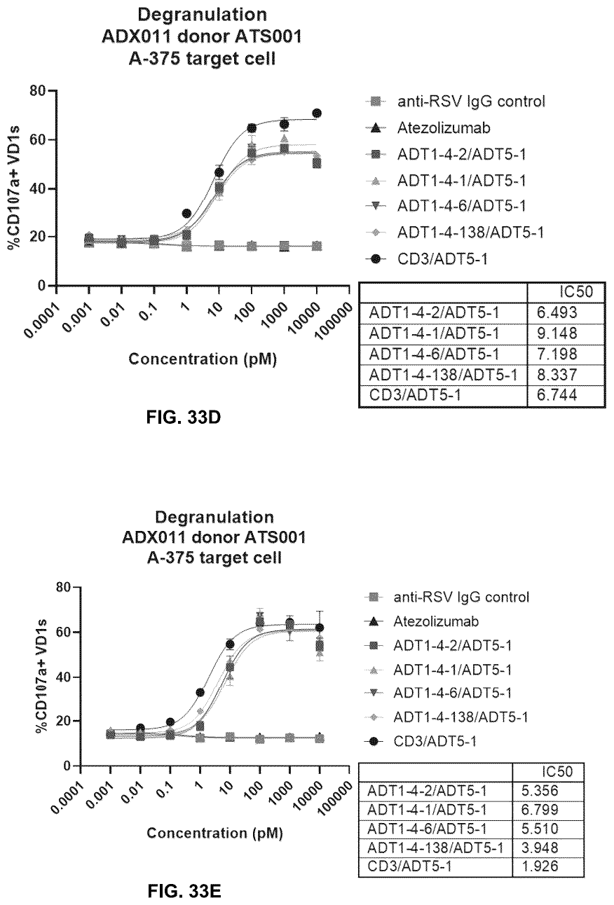

Adherent tumor cells (A431 PD-L1^{high}, A-549 PD-L1^{low} and A-549 PD-L1^{KO}) cells were detached from tissue culture flasks and re-suspended in basal growth media and seeded in 96-well plates at the relevant cell dilutions and allowed to adhere for 4 h. Subsequently, expanded skin derived Vδ1 γδ T cells were detached from tissue culture flasks, re-suspended in basal growth media and seeded in 96-well plates at the relevant cell dilutions to achieve a 1:1 E:T ratio with previously seeded target cells. Antibodies were diluted and added to each well at the specified concentration. The cultures were then incubated at 37° C., 5% CO2 for 244 hours. To measure degranulation, a fluorophore-conjugated anti-CD107a antibody was added directly into the cell-antibody mix at the start of the 24-hour co-culture. Following, two washes and cell fixation, fluorescence was measured using the MACSQuant® and live cell counts, and median fluorescence intensity determined. The results are shown in FIGS. 33A-33C and EC50s summarised in Table 34.

TABLE 34

| | ADT5-1 | | | | | CD3/PD-L1 | | | Anti-RSV isotype control | | |
|---|---|---|---|---|---|---|---|---|---|---|---|
| | A-431 | | A-549 | | A-549 | A-431 | A-549 | A-549 | A-431 | A-549 | A-549 |
| Donor | PD-L1^{high} | | PD-L1^{low} | | PD-L1^{KO} | PD-L1^{high} | PD-L1^{low} | PD-L1^{KO} | PD-L1^{high} | PD-L1^{low} | PD-L1^{KO} |
| ATS036 | 20 | pM | 11.7 | pM | >1000 pM | 7.6 pM | 7.2 pM | >1000 pM | >1000 pM | >1000 pM | >1000 pM |
| ATS037 | 18.4 | pM | 7.1 | pM | >1000 pM | 7.5 pM | 4.1 pM | >1000 pM | >1000 pM | >1000 pM | >1000 pM |

| | | | | | | | | | |
|---|---|---|---|---|---|---|---|---|---|
| Mean | 19.2 pM ± | 9.4 pM ± | >1000 pM | 7.6 pM ± | 5.7 pM ± | >1000 pM | >1000 pM | >1000 pM | >1000 pM |
| | 1.1 pM | 3.3 pM | | 0.1 pM | 2.2 pM | | | | |

Conclusions: This data demonstrates that the Vδ1/PD-L1 bispecific antibodies induce the degranulation of primary Vδ1-positive γδ T-cells in a PD-L1 dependent manner.

### Example 11. Assessing Effect of Anti-Vδ1 Affinity on γδ T Cell Activation and Degranulation In Vitro

Expanded skin-derived Vδ1 γδ T-cells and PD-L1+ tumor cells (A-375) were detached from tissue culture flasks and re-suspended in basal growth media and seeded in 96-well plates at the relevant cell dilutions to achieve a 1:1 E:T ratio. Adherent tumor cells (A431 PD-L1^{high}, A-549 PD-L1^{low} and A-549 PD-L1^{KO}) cells were detached from tissue culture flasks and re-suspended in basal growth media and seeded in 96-well plates at the relevant cell dilutions and allowed to adhere for 4 h. Subsequently, expanded skin derived Vδ1γδ T cells were detached from tissue culture flasks, re-suspended in basal growth media and seeded in 96-well plates at the relevant cell dilutions to achieve a 1:1 E:T ratio with previously seeded target cells. Antibodies were diluted and added to each well at the specified concentration. The cultures were then incubated at 37° C., 5% CO2 for 4 hours. To measure degranulation, a fluorophore-conjugated anti-CD107a antibody was added directly into the cell-antibody mix at the start of the 4-hour co-culture. Following, two washes and cell fixation, fluorescence was measured using the MACSQuant® and live cell counts, and median fluorescence intensity determined. The results are shown in FIGS. 33D and 33E.

Conclusions: This data demonstrates that the Vδ1/PD-L1 bispecific antibodies with different anti-Vδ1 affinities ranging from <1 nM to 60 nM induce a similar level of degranulation of primary Vδ1-positive γδ T-cells in a PD-L1 dependent manner

### Example 12. Assessing Vδ1 γδT Cell-Dependent Cellular Cytotoxicity (TDCC)

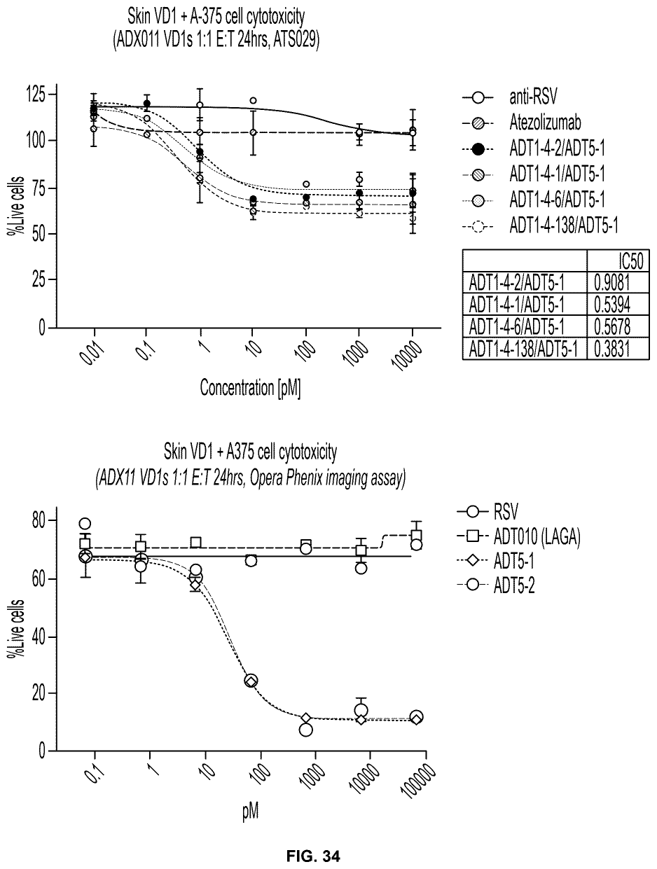

Adherent PD-L1+ tumor A-375 (nucGreen®, GFP+) cells were detached from tissue culture flasks and re-suspended in basal growth media and seeded in 384-well CellCarrier Ultra (PerkinElmer®) imaging plates and allowed to adhere for 4 hours. Subsequently, expanded skin-derived Vδ1 γδ T-cells were detached from tissue culture flasks and added to culture at the relevant cell dilutions to achieve a 1:1 E:T ratio. E. Antibodies were diluted and added to each well at a top concentration of 100 nM. The cultures were then incubated at 37° C., 5% CO2 for 24 hours. To measure the remaining live target cells, images were acquired on an Opera Phenix® confocal imaging system (PerkinElmer®) and quantity of viable target cells determined using a linear regression model (Harmony 4.9) based on size, morphology, texture and intensity of live GFP+ cells. The results are shown in FIG. 34.

Conclusions: Specifically, no significant difference in PD-L1+ target cell lysis by skin-Vδ1 s in presence of ADT5-1 was observed with different anti-Vδ1 affinities. Similarly, no significant difference in PD-L1+ target cell lysis by skin-Vδ1s in presence of ADT5-1 or ADT5-2 anti-PD-L1 Fcabs was observed. This data demonstrates that the Vδ1/PD-L1 bispecific antibodies with different anti-Vδ1 affinities ranging from <1 nM to 60 nM enhance the cytolytic ability of primary Vδ1-positive γδ T-cells to a similar extent in a PD-L1 dependent manner.

### Example 13. Cytolytic Potential of Both Blood-Derived and Skin-Derived Vδ1 γδT Cells is Enhanced

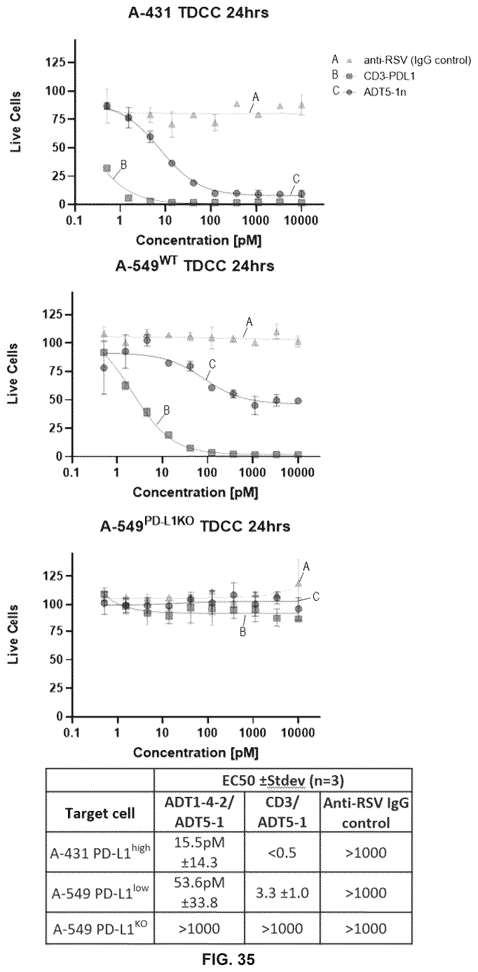

by Vδ1/PD-L1 bispecific antibodies in a PD-L1 dependent manner (A) Cytolytic potential of blood-derived Vδ1 γδT cells. PBMCs were pre-expanded for 7 days with 10 μM ADT1-4-2/ADT5-1 prior to employing in tumor cell cytotoxicity assays. PD-L1⁺ tumor cells were detached from tissue culture flasks and stained with CellTrace™ CFSE live cell dye (Life Technologies®), before resuspending in basal growth media and seeding in 384-well CellCarrier Ultra plates (PerkinElmer®) imaging plates and incubating at 37° C., 5% CO2 for 24 hours. ADT5 pre-expanded PBMCs were collected, washed and resuspended in basal growth media and seeded into 384-well imaging plates to achieve a co-culture at a 40:1 PBMC:tumor cell ratio, in the presence of a titration of ADT1-4-2/ADT5-1, a CD3/ADT5-1 control, or anti-RSV IgG control. The co-cultures were then incubated at 37° C., 5% CO2 for 24 hours. To measure the remaining live target cells, cells were stained with Hoechst 33342 (Invitrogen®) for 1 hour at 37° C. before acquiring images on an Opera Phenix® confocal imaging system (PerkinElmer®) and quantity of viable target cells determined using a linear regression model (Harmony® 4.9) based on size, morphology, texture and intensity of live Hoechst⁺/CFSE⁺ cells. The results are shown in FIG. 35. After 7-day pre-stimulation with ADT1-4-2/ADT5-1, CD3⁺/αβTCR⁺ cells comprise approximately 83% and CD3+/Vδ1TCR+ cells comprise approximately 8.6% of the total PBMC population hence the enhanced potency exhibited by the CD3/ADT5-1 engager.

Conclusions: This data demonstrates that blood-derived Vδ1 γδT cell cytolytic potential is enhanced by Vδ1/PD-L1 bispecific antibodies in a PD-L1 dependent manner correlating with the level of cell surface expression of PD-L1.

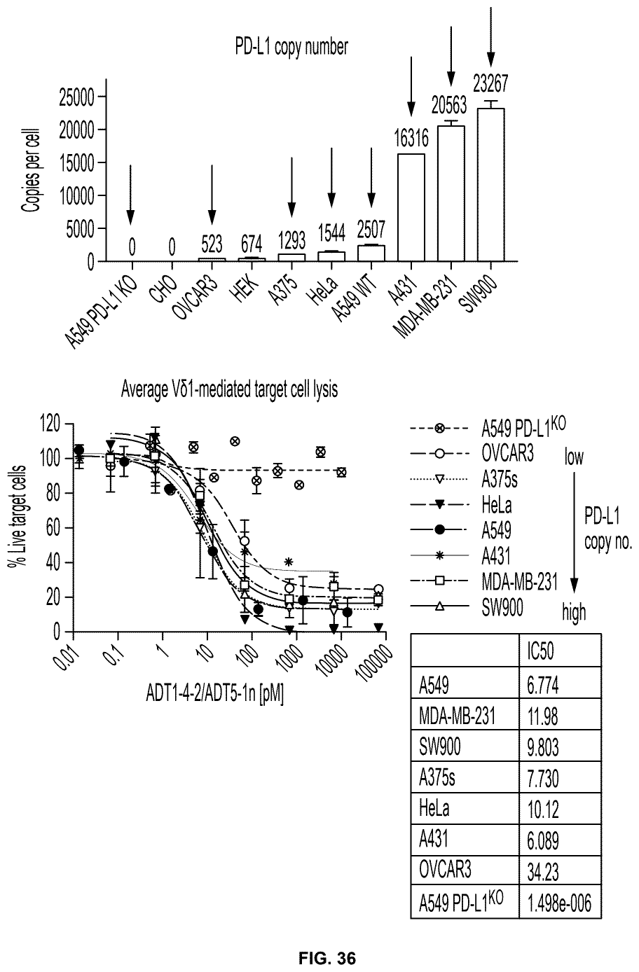

(B) Cytolytic potential of skin-derived Vδ1 γδT cells. Expanded skin-derived Vδ1 γδ T-cells and PD-L1⁺ tumor cells were detached from tissue culture flasks and re-suspended in basal growth media and seeded in 384-well CellCarrier Ultra plates (PerkinElmer®) imaging plates at the relevant cell dilutions to achieve a 1:1 E:T ratio. Antibodies were serially diluted and added to each well. The co-cultures were then incubated at 37° C., 5% CO2 for 24 hours. To measure the remaining live target cells, cells were stained with Hoechst 33342 (Invitrogen®) for 1 hour at 37° C. before acquiring images on an Opera Phenix® confocal imaging system (PerkinElmer®) and quantity of viable target cells determined using a linear regression model (Harmony® 4.9) based on size, morphology, texture and intensity of live Hoechst+ cells. The results are shown in FIG. 36.

Conclusions: This data demonstrates that skin-derived Vδ1 γδT cell cytolytic potential is enhanced by Vδ1/PD-L1 bispecific antibodies in a PD-L1 dependent manner providing that there is a detectable level of cell surface PD-L1 expression

### Example 14: Assessing Pharmacokinetics of Potential Lead Vδ1-PD-L1 Bispecific Antibodies

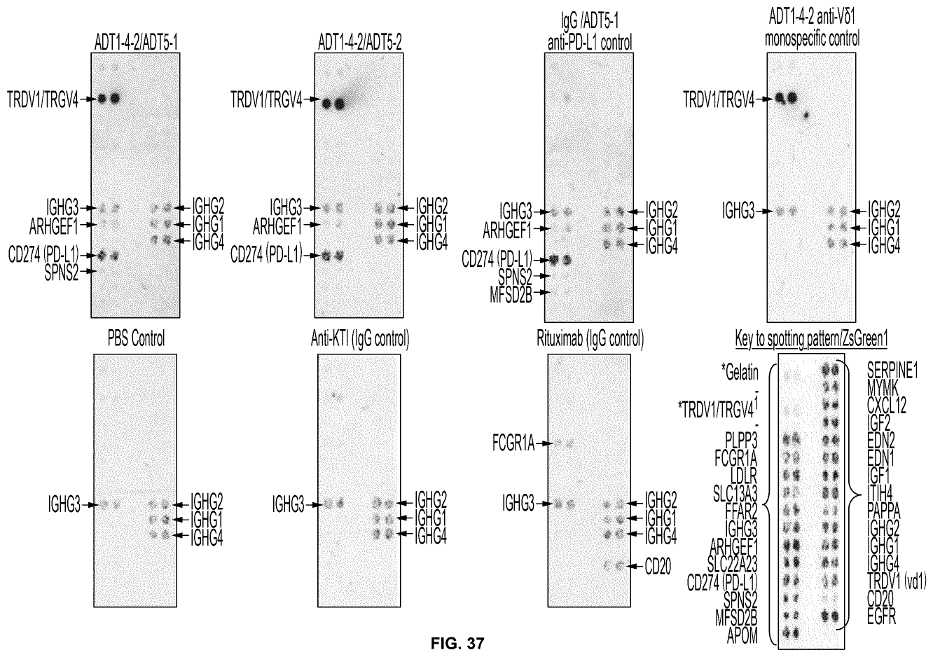

To determine the specificity of VD1/PD-L1 bispecifics a binding assessment was performed on ADT1-4-2/ADT5-1 and ADT1-4-2/ADT5-2 using Retrogenix® Cell Microarray Technology performed by Charles River®. Following a pre-screening step to confirm successful binding to recombinant human Vδ1/Vγ4 and PD-L1, antibodies underwent a library screen to assess binding to 6,101 plasma membrane, secreted or cell surface-tethered human proteins, and 396 human heterodimers. Positive hits were further confirmed in a subsequent confirmation screen as shown in FIG. 37.

Conclusions: ADT1-4-2/ADT5-1 and ADT1-4-2/ADT5-2 both showed a significant specific interaction with CD274 (PD-L1), in both fixed and live microarrays, and a significant specific interaction with recombinant Vδ1/Vγ4 protein spotted in gelatin. No other interactions were identified indicating a high specificity for the primary targets, human Vδ1 and PD-L1. In vivo pharmacokinetics is not anticipated to be different for ADT5-1 & ADT5-2.

### Example 15. Safety and PD-L1 Related Toxicities

PD-L1 blockade therapy can cause a variety of toxicities when administered in vivo. PD-L1 blockade is a clinically validated mechanism of action in sensitive populations.

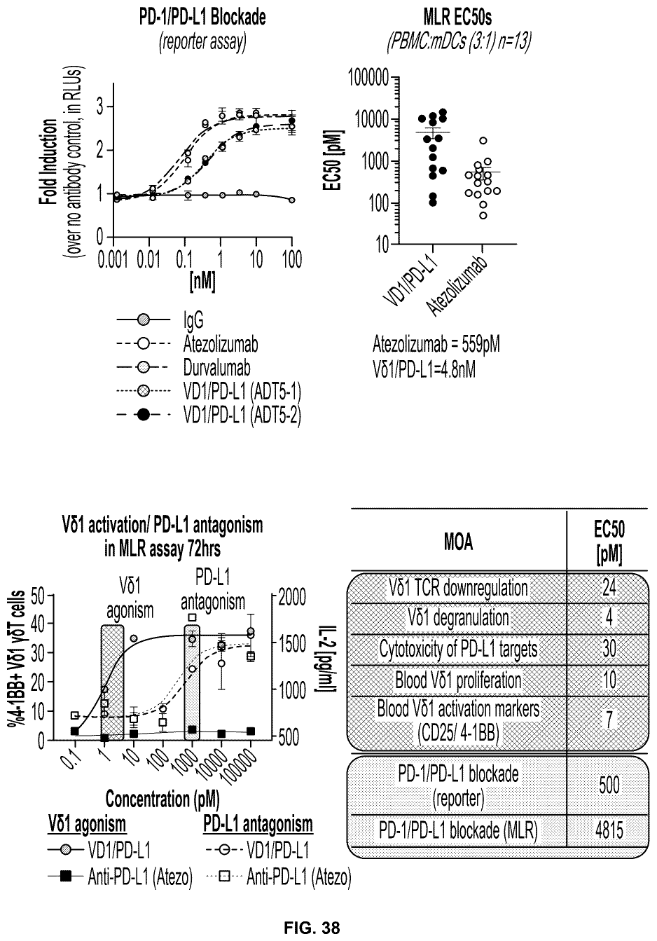

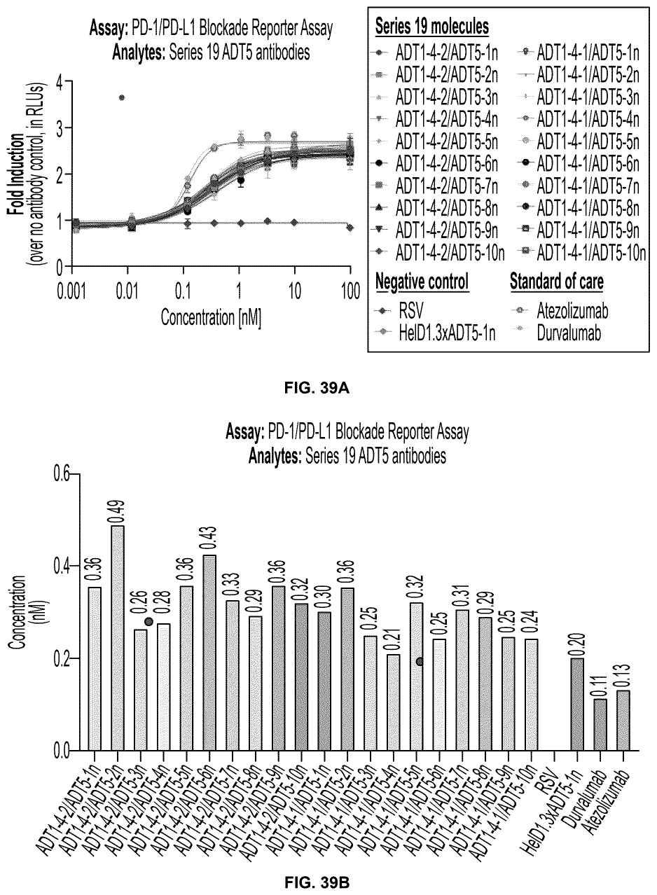

The antibodies of the disclosure are Vδ1 agonists at pM range and PD-L1 antagonists at nM range. PD-L1 blockade is unlikely to be achieved at anticipated clinical doses. Effective blockade is achieved at concentrations ˜10× higher than PD-L1 SoC (Atezolizumab/Durvalumab) and ˜100× higher than that required for Vδ1 agonism (see FIG. 38). This highlights the role of the Vδ1×PD-L1 engagers of the disclosure in enabling peripheral Vδ1 activation for PDL1+ cells without blocking the PD-L1/PD-1 pathway since such a blocking effect would occur at much higher concentrations than the priming and agonist effects.

Decoupling functional activation of Vδ1 cells from blockade of PD-L1 pathways enables activation of key Vδ1 anti-tumor functionality while avoiding potential additional deleterious effects of off-tumor PD-L1 blockade, as well as providing a clear biological rationale for using Vδ1-PDL1 engagers in patients who are PD-L1 blockade resistant.

Conclusions: PD-L1 inhibition related toxicities are not expected with the antibodies of the disclosure.

### Example 16. PD-1/PD-L1 Blockade Reporter Bioassay Using Vδ1-PD-L1 Bispecific Antibodies

A PD-1/PD-L1 reporter assay (Promega®) was utilised to assess the ability of anti-Vδ1/PD-L1 bispecific antibodies to block the PD-1/PD-L1 axis. PD-1 effector cells (Luc-NFAT-RE⁺ Jurkat) and PD-L1⁺ aAPC/CHO-K1 cells were seeded into white 96-well plates in the presence of a titration of Vδ1/PD-L1 antibodies with a top final concentration of 100 nM. After a 6-hour co-culture, the ability of the Vδ1/PD-L1 antibodies to block the PD-1/PD-L1 axis was assessed by measuring luminescence on a plate reader after the addition of Bio-Glo™ reagent. The results are shown in FIG. 39A and EC50s compared in FIG. 39B.

Conclusions: All anti-PD-L1 Fcabs exhibited similar potency in the PD-1/PD-L1 reporter assay. Different anti-Vδ1 Fab sequences on the anti-PD-L1 Fcabs did not affect their potency. All Fcabs exhibited EC50s between 0.21 nM and 0.49 nM.

### Example 17. Manufacturability & Stability Assessment of Vδ1-PD-L1 Bispecific Antibodies

To better understand the manufacturability/stability of the novel Vδ1-PD-L1 bispecific molecules as described herein the inventors assessed a panel of these molecules via a variety of methods. In particular, two candidates, ADT5-1 and ADT5-2, were assessed to determine which molecule has a more favourable profile.

The two ADT5 mAb candidates, ADT5-1 and ADT5-2 were found to be comparable in regards of most attributes tested. Further CMC (Chemistry, Manufacturing, and Controls) assays were carried to determine which candidate should be designated as the lead candidate.

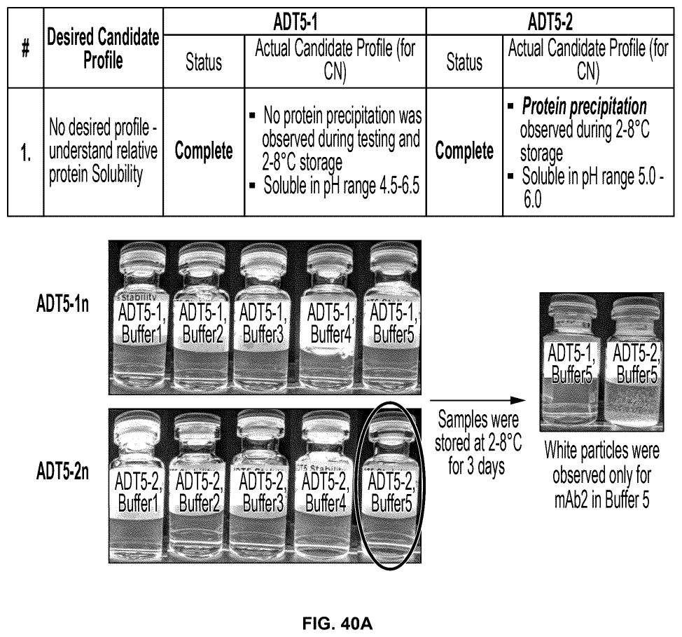

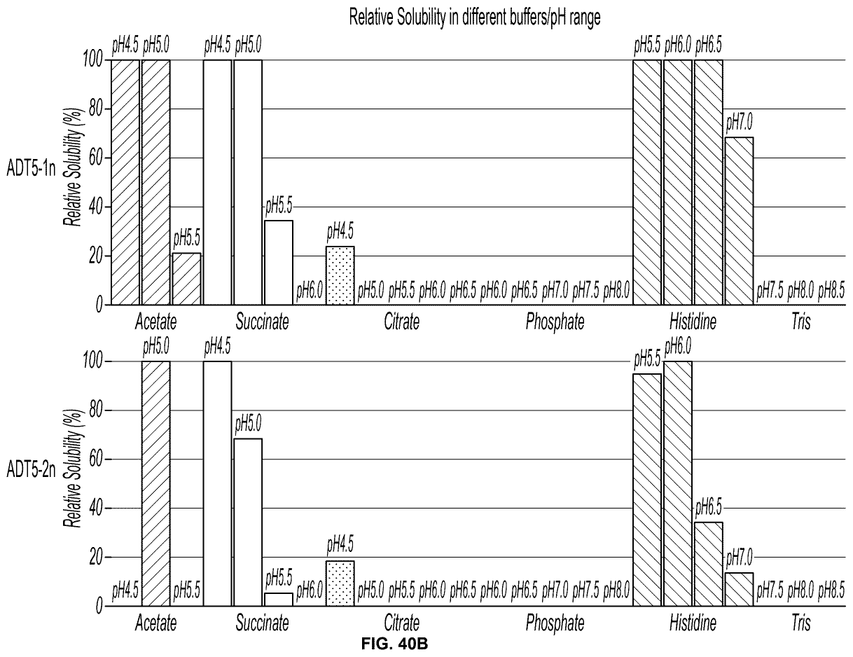

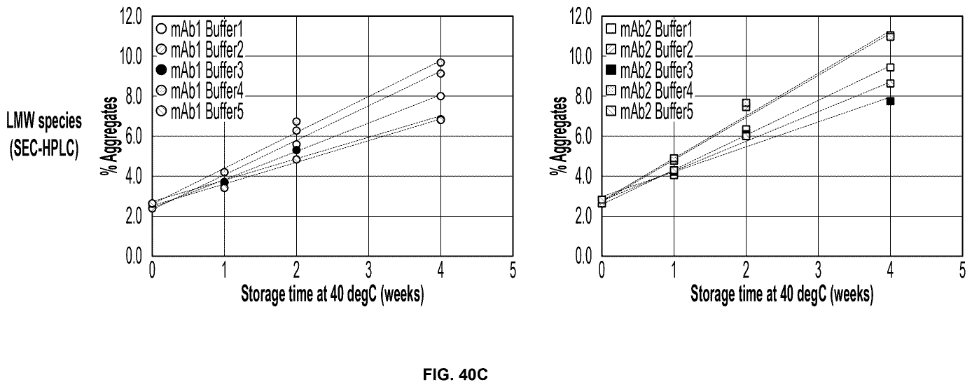

Manufacturability assessments on the Vδ1/PD-L1 bispecific antibodies ADT1-4-2/ADT5-1 and ADT1-4-2/ADT5-2 were performed in different buffers and at a range of pHs to assess their stability and manufacturability. Vδ1/PD-L1 bispecific antibodies underwent buffer exchange before assessing the relative solubility by methods known in the art. Antibodies were stored at 40° C. for up to 4 weeks and the presence of LMW species determined by SEC-HPLC as a measure of aggregation (see FIGS. 40A-40C).

Conclusions: ADT1-4-2/ADT5-1 demonstrated minimal precipitation and greater solubility in a wider pH range (4.5-6.5) compared to ADT1-4-2/ADT5-2 (pH 5-6) (see FIGS. 40A-40C). For ADT1-4-2/ADT5-1 no protein precipitation was observed during storage and testing for 10 mg/mL protein concentration. ADT1-4-2/ADT5-1 demonstrated a lower rate of degradation/aggregation at 40° C. storage up to 4 weeks and a lower rate of change in charge profile at 40° C. storage up to 4 weeks compared to ADT1-4-2/ADT5-2. Additionally ADT1-4-2/ADT5-2 demonstrated a relatively higher rate of aggregation in thermal stress studies over 4 weeks compared to ADT1-4-2/ADT5-1. ADT1-4-2/ADT5-1 demonstrated a slightly higher yield during tests of manufacturing processes. Although of course other factors may be important when designating a lead, CMC data supports proceeding with candidate ADT5-1 as the lead (with ADT5-2 as backup if required).

### Example 18. Stimulation with Vδ1-PD-L1 Bispecific Antibodies Enhances Blood Vδ1 Proliferation

As described herein, one advantageous property of the Vδ1-PD-L1 multispecific antibodies of the disclosure is that they can provoke peripheral Vδ1 γδ T cell proliferation and priming via crosslinking to PD-L1+ normal immune cells, i.e. in a tumour free system. This example evidences how stimulation with Vδ1-PD-L1 bispecific antibodies enhances Vδ1 proliferation and priming in the blood.

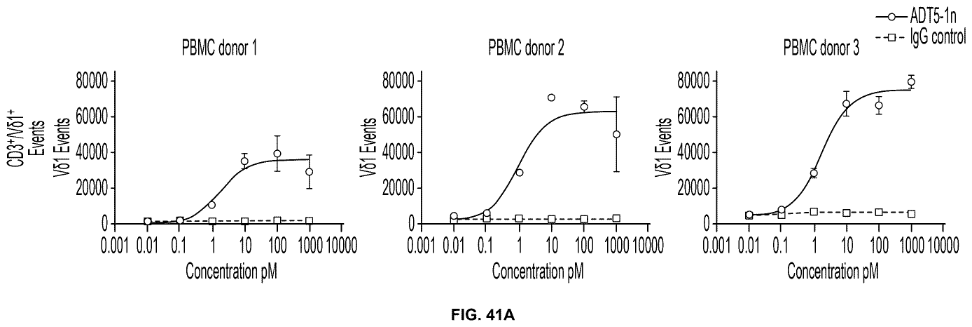

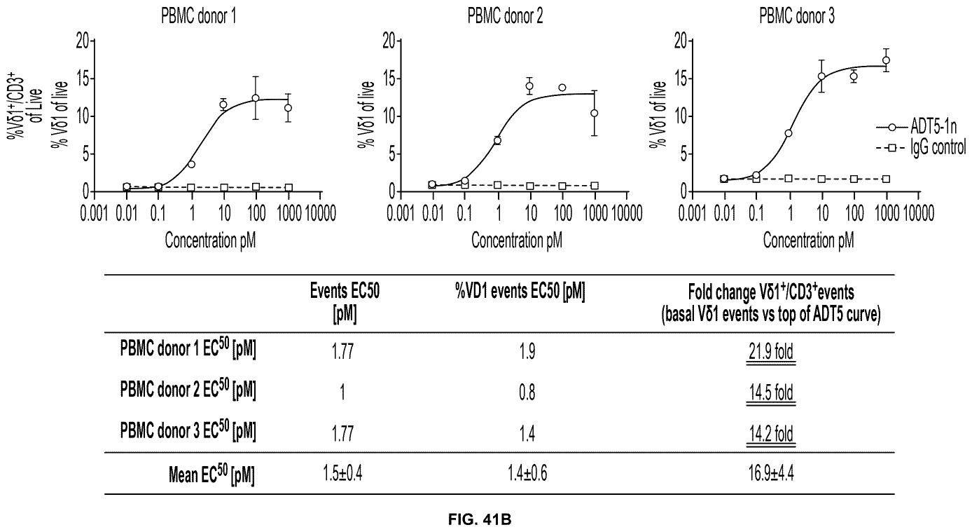

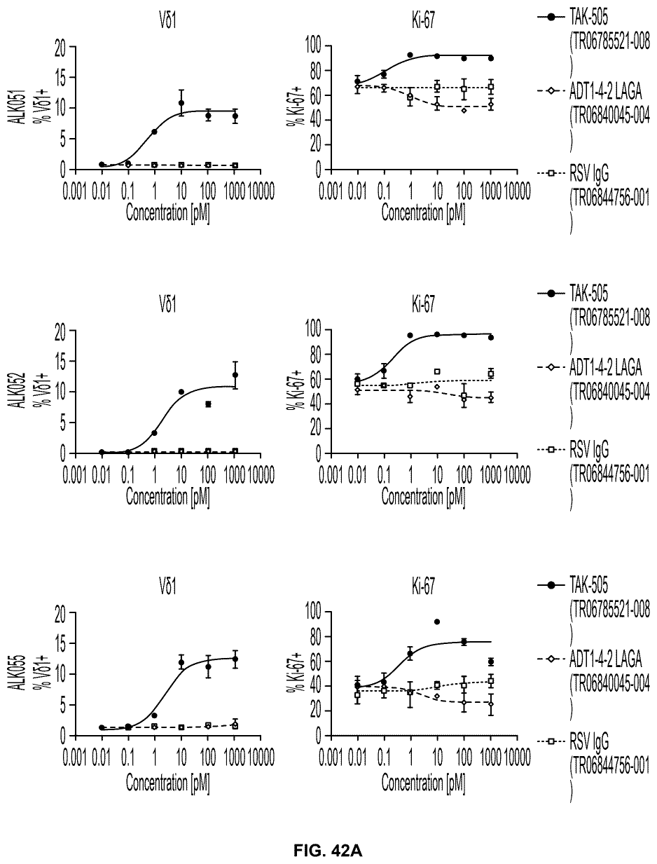

PBMCs from healthy donors were stimulated with a titration of ADT1-4-2/ADT5-1 for 7 days at a top concentration of 1 nM. PBMCs were seeded at 2×10\hat{ }6 cells per well in 48-well plates in basal growth media in the presence of ADT1-4-2/ADT5-1 or IgG control before incubating at 37° C., 5% CO2 for 7 days. Cells were collected and stained with anti-CD3, anti-CD4, anti-CD14, anti-CD19, anti-CD56, anti-Vδ1, anti-Vδ2, anti-αβTCR, anti-CD45RA and anti-CD27 before assessing on a MACSquant® 16 Flow Cytometer (Miltenyi Biotec®) to determine the composition of the PBMCs and quantify different immune subsets. The number of Vδ1 γδT cell events and percent of live PBMCs is shown in FIGS. 41A-41B.

Conclusions: This data demonstrates that Vδ1/PD-L1 bispecific antibodies can surprisingly induce the proliferation of peripheral blood Vδ1 γδT cells, even in the absence of adherent PD-L1⁺ cells or without administration of exogenous growth cytokines.

### Example 19. Stimulation with Vδ1-PD-L1 Bispecific Antibodies Induces Proliferation and Activation of Peripheral Vδ1 γδ T Cells

The objective of this study was to evaluate the ability of antibodies of the disclosure to induce the proliferation and activation of peripheral Vδ1 γδ T cells in vitro, as well as priming said Vδ1 cells. Human peripheral blood mononuclear cells (PBMCs) from five donors were treated with antibodies of the disclosure (TAK-505, ADT1-4-2/ADT5-1) or control test articles (anti-Vδ1 or an anti-respiratory syncytial virus [RSV]) for 7 days. The fold-increase in absolute Vδ1 γδ T cell counts and half maximal effective concentration (EC50) values of Vδ1 γδ T cell proliferation and activation were determined via flow cytometry.

### Materials and Methods:

Human peripheral blood mononuclear cells (PBMCs) from five donors were thawed and resuspended in pre-warmed PBMC culture media. Following recovery, PBMC cell suspensions were collected and cell counts were measured and adjusted to 5×106 PBMCs/mL using PBMC culture media. PBMCs were seeded in 48-well tissue culture plates at a density of 2×106 cells/400 uL/well. Antibodies of the disclosure (TAK-505, ADT1-4-2/ADT5-1) and control test articles (anti-Vδ1 antibody (ADT1-4-2 LAGA), and anti-RSV antibody (TR06844756-001) were added to respective wells in concentrations of 1000, 100, 10, 1, 0.1 and 0.01 picomolar concentration in 400 uL PBMC culture media.

Treated PBMCs were then cultured under standard culture conditions at 37° C./5% CO2 for six days. On day 6, Vδ1 detection antibody (REA173 clone; Miltenyi Biotec®, 130-120-440) was added to each well to stain the Vδ1 γδT cells. 48-well tissue culture plates were then placed back into standard culture conditions at 37° C. with 5% CO2 for an additional 24 hours. All test article conditions were tested as triplicate sample wells across 3 separate plates.

PBMC phenotype was assessed by flow cytometry (BD® LSRFortessa™ (Becton Dickinson, Franklin Lakes, NJ, USA) at Day 0 and Day 7. PMBC suspensions were harvested and stained at 4° C. with fluorochrome-labeled anti-TCRγ/δ, anti-CD274 (PD-L1), anti-CD19, anti-TCR Vδ1, anti-CD27, anti-CD56, anti-CD14, anti-TCR Vδ2, anti-CD3, anti-CD45, anti-CD4, anti-TCR ap, anti-CD45RA diluted in eBioscience™ Flow Cytometry Staining Buffer and BD® Horizon™ Brilliant Stain Buffer.

Flow cytometry was utilized to evaluate the activity of antibodies of the disclosure (TAK-505, ADT1-4-2/ADT5-1) and control test articles on proliferation and activation markers in Vδ1 γδT cells (day 0 and 7). For day 7 samples, cell suspensions were harvested, washed and stained with Fixable Viability Dye eFluor780 (Thermo Fisher Scientific®) and Fc-blocking reagent (Miltenyi Biotec®). Cells were then washed and stained at 4° C. with fluorochrome-labeled anti-TCRγ/δ, anti-CD19, anti-TCR Vδ1, anti-CD137 (4-1 BB), anti-CD3, anti-CD45 and anti-NKG2D diluted in eBioscience™ Flow Cytometry Staining Buffer and BD® Horizon™ Brilliant Stain Buffer. Stained cells were the washed fixed and permeabilized before acquiring samples on BD FACSymphony® (Becton Dickinson, Franklin Lakes, NJ, USA) per manufacturer's instructions.

Cells for flow cytometry analysis were analyzed with two flow cytometry gating strategies to determine baseline (day 0) Vδ1 γδT cell content and to evaluate the activity of TAK-505 and control test articles on the frequency (%+) of CD27 and to evaluate the activity of TAK-505 and control test articles on the frequency (%+) of Vδ1+γδT cells, Ki-67 and NKG2D in this population. Additional data analysis of flow cytometry data was performed using Microsoft® Excel and GraphPad® Prism Analyzed flow cytometry data were plotted and nonlinear regression curves were performed to generate concentration-response curves. EC50 values for Vδ1 γδT cell proliferation and the markers Ki-67, NKG2D, and CD27 were also determined using these curves. In cases where concentration-response curves did not result in accurate regression curve fitting, EC50 values were reported as >1000 pM. Statistical analysis was performed using GraphPad® Prism for MacOS (version 10.2.3). The effect of TAK-505 and control test articles on absolute Vδ1 γδT cell numbers and fold change in growth across all donors was assessed by an ordinary two-way ANOVA (Analysis of Variance) with a Tukey's multiple comparison test and a single pooled variance (95% confidence interval). A p-value<0.05 was considered statistically significant.

A cell viability analyzer was utilized to evaluate the activity of antibodies and control test articles on absolute Vδ1 γδT cell numbers and fold growth in all donors using a Vi-CELL BLU Cell Viability Analyzer (Beckman Coulter®). Absolute Vδ1 γδT cell counts were determined mathematically using Microsoft Excel by multiplying the frequency of Vδ1+ cells on day 0 and day 7 (acquired by flow cytometry described above) and the total day 0 seeded cell counts and total day 7 viable cell counts from the cell viability analyzer).

TABLE 35
EC₅₀ of TAK-505 mediated increase of Vδ1 γδ
T cell frequency and increased frequency of Vδ1 surface
expression of Ki-67, NKG2D and CD27.

| | Vδ1 | Ki-67 | NKG2D | CD27 |
|---|---|---|---|---|
| Donor | EC₅₀ (pM) | EC₅₀ (pM) | EC₅₀ (pM) | EC₅₀ (pM) |
| ALK051 | 0.55 | 0.13 | 0.22 | 0.2 |
| ALK052 | 2 | 0.19 | 0.27 | 0.1 |
| ALK055 | 2.39 | 0.33 | 0.57 | 1.84 |
| ALK056 | 2.43 | 0.2 | 0.67 | 68.99 |
| ALK057 | 1.3 | 0.1 | 0.17 | 0.12 |
| Mean | 1.73 | 0.19 | 0.38 | 14.25 |
| | ±0.8 | ±0.09 | ±0.22 | ±30.61 |

### Results:

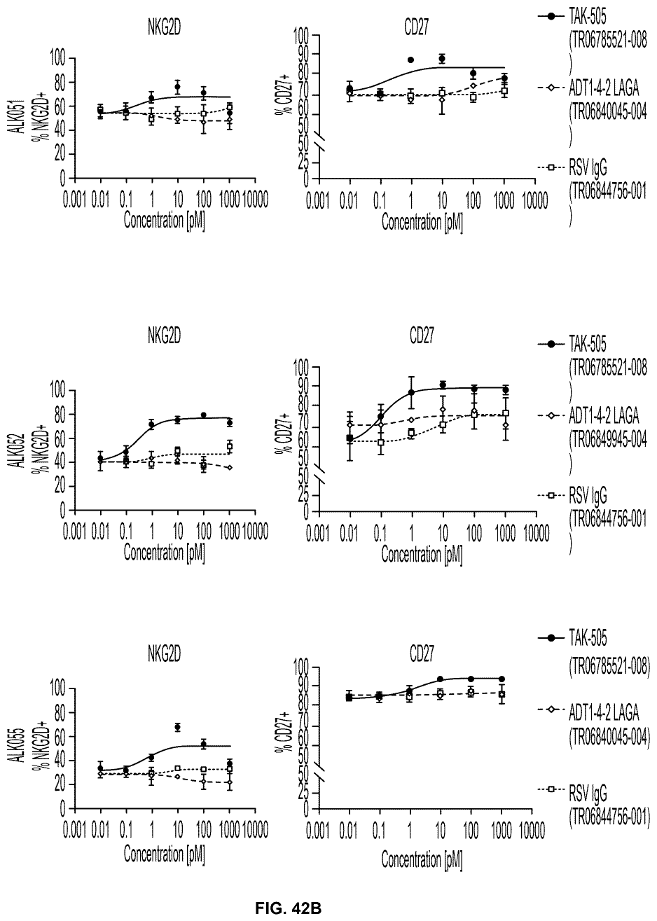

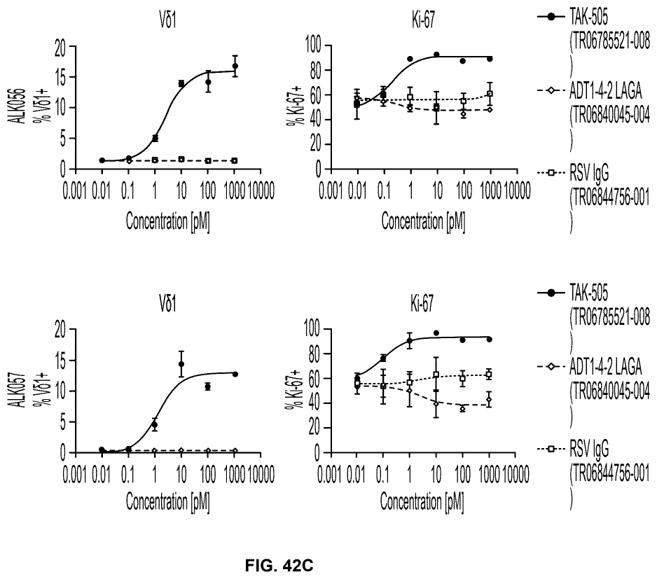

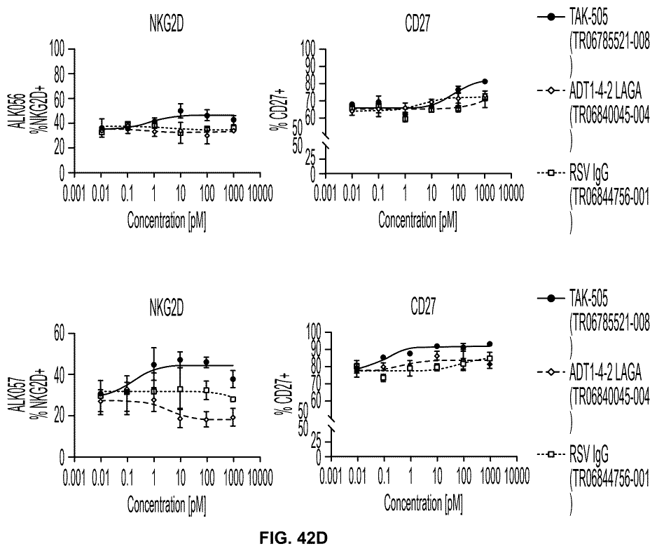

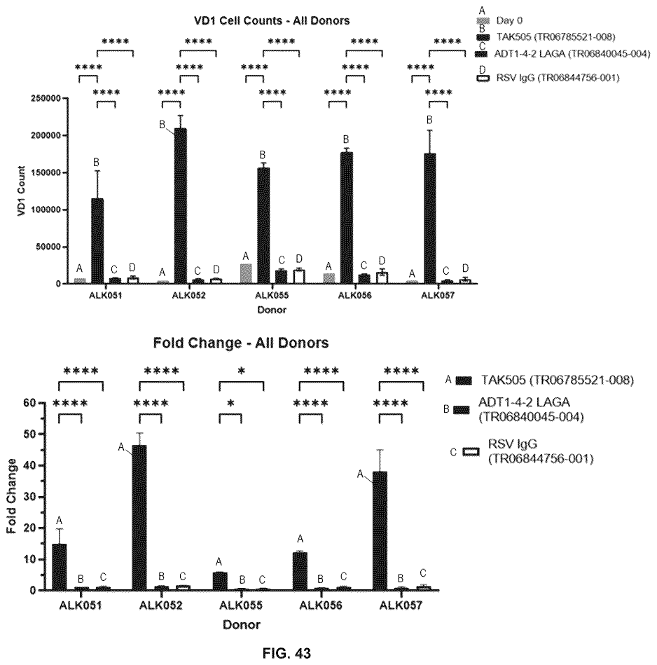

When treated with antibodies of the disclosure (TAK-505, ADT1-4-2/ADT5-1), all 5 PBMC tested donors exhibited Vδ1 γδ T cell proliferation over 7 days of culture. Mean fold change in absolute Vδ1 γδT cell counts ranged from 5.86±0.23 (donor ALK055) to 48.79±4.00 (donor ALK052) at low picomolar concentrations (average EC50 of 1.73±0.8 μM). Additionally, TAK-505 lead to an increase in Ki-67, NKG2D and CD27 frequency on Vδ1 γδT cells, with a mean EC50 of 0.19 μM, 0.38 μM, and 14.25 μM respectively. In contrast, anti-RSV and anti-Vδ1 control test articles exhibited minimal effects on Vδ1 γδT cell proliferation or on the frequency of Ki-67, NKG2D or CD27 on Vδ1 γδT cells. Blood Vδ1 γδT cell proliferation and the frequency of cell surface markers associated with activation by TAK-505 treatment is depicted graphically in FIGS. 42A-42D, FIG. 43 and Table 35.

### Conclusion:

Antibodies of the disclosure (TAK-505, ADT1-4-2/ADT5-1) induce peripheral blood Vδ1 γδ T cell proliferation. Additionally, antibodies of the disclosure (TAK-505, ADT1-4-2/ADT5-1) lead to an increase in Ki-67, NKG2D and CD27 frequency on blood Vδ1 γδT cells, demonstrating their priming effect.

### Example 20. Evaluation of the In Vitro Activity of Vδ1×PD-L1 Bispecific on Pro-Inflammatory Cytokine and Chemokine Secretion

This example focuses on the evaluation of the in vitro activity of Vδ1×PD-L1 bispecific on pro-inflammatory cytokine and chemokine secretion from skin derived human Vδ1 γδ T cells.

The objective of this example was to evaluate the ability of antibodies of the disclosure (TAK-505, ADT1-4-2/ADT5-1) to mediate the production of cytokine and chemokine factors from tissue-derived immune cells cultured with PD-L1+ tumor cell targets. In vitro cultured skin-derived immune cells enriched for Vδ1 γδ T cells through γδ T cell depletion were cocultured with commercially available PD-L1+ tumor cell targets A375 (malignant melanoma) (or A431 (epidermoid carcinoma)—data not shown), with cultures stimulated with antibodies of the disclosure or either a single-specificity Vδ1-binding control (ADT1-4-2) or a CD3×PD-L1 dual specificity positive control engager. Test and control articles were tested a dose-titration range of approximately 0.1-1000 pM.

### Materials and Methods:

Leukocytes (comprising approximately 0.1-2% Vδ1 γδT cells) were isolated from healthy human skin samples. The cells were suspended and analysed by flow cytometry to establish cell composition and phenotype. The remaining cell suspension was enriched for Vδ1 γδ T cells via ap-depletion prior to co-culture assays with target cells to evaluate cytokine and chemokine release.

Serial dilutions of TAK-505 and the controls (CD3×PD-L1 and ADT 1-4-2 LAGA) were prepared and 50 μL of each cell engager was added to 150 μL of target cells plus effector cells to ensure that the concentrations of TAK505 was added at 972.61 pM, 97.26 pM, 9.73 pM, 0.97 pM, 0.097 pM and 0.0097 pM final concentration, and engager controls were 1000 pM, 100 pM, 10 pM, 1 pM, 0.1 pM and 0.01 pM for the assay.

The human skin-derived immune cell populations depleted of aPT cells were co-cultured with A-375 or A-431 target cells at an effector: target (E:T) ratio of 2:1 with TAK-505 or a control article A six-point titration was performed for test concentrations to be 1000 pM, 100 pM, 10 pM, 1 pM, 0.1 pM, and 0.01 pM for all test and control articles. Total viable cell counts measured using a NucleoCounter® NC-200 (ChemoMetec®) before co-cultures were then incubated under standard culture conditions at 37° C./5% CO2. After 24 hours, cell supernatants were harvested and underwent electrochemiluminescence-based quantitation using MSD technology to determine the quantities of select cytokine and chemokine analytes. Analyte quantitation data was captured and analyzed using MSD Discovery Workbench® software (MSD, USA). The fold-difference in production of each soluble analyte between TAK-505 stimulated cultures versus concentration control ADT1-4-2 cultures was calculated.

### Results:

In co-cultures of skin derived immune cells and the A375 tumor target cell line, antibodies of the disclosure (TAK-505, ADT1-4-2/ADT5-1) elicited the production of IFNγ (EC50=11.9 pM±2.4 SD), IL-6 (EC50=6.7 pM±2.2 SD), IL-8 (EC50=6.6 pM±3.5 SD), TNFα (EC50=12.2 pM±3.7 SD), GM-CSF (EC50=5.9 pM±3.6 SD), MIP-1β (EC50=8.1 pM±1.8 SD), IP-10 (EC50=11.7 pM±1.3 SD), MIP-1α (EC50=8.5 pM±1.0 SD) and MCP-1 (EC50=5.8 pM±1.7 SD). In cocultures of skin derived immune cells and the A431 tumor target cell line (data not shown), TAK-505 elicited the production of IFNγ (EC50=22.2 pM±8.7 SD), IL-6 (EC50=19.2 pM±18.4 SD), IL-8 (EC50=9.9 pM±7.3 SD), TNFα (EC50=8.4 pM±5.8 SD), GM-CSF (EC50=15.6 pM±20.8 SD), MIP-1β (EC50=7.6 pM±2.0 SD), IP-10 (EC50=12.9 pM±2.3 SD), MIP-1α and (EC50=8.9 pM±2.3 SD). For all tested coculture conditions, TAK-505 did not elicit the production of IL-17A, IL-10 or IL-1P above 1.2-fold versus ADT1-4-2 control conditions. In summary, TAK-505 enhances the production of key proinflammatory cytokine and chemokine factors from tissue-derived immune cell cultures challenged with PDL1+ tumor targets, while also being unable to stimulate the production of IL-17A or IL-1p, and limited capacity to stimulate the production of IL-10, from skin-derived immune cells. This is shown in FIGS. 44A-44B.

### Conclusion:

In this study, antibodies of the disclosure (TAK-505, ADT1-4-2/ADT5-1) stimulation elicited the production of key proinflammatory factors from skin-derived immune cell cultures compared to ADT1-4-2 control cultures across a broad range of analytes. This highlights the potential for antibodies of the disclosure to activate tissue-based Vδ1 γδ T cells and to propagate a wider immune response through recruitment of other immune cells and proinflammatory conditioning of the local tissue area. Furthermore, the data in this study demonstrate the lack of ability for TAK-505 to stimulate production of IL-17A and IL-10 cytokines.

### Example 21. Stimulation with Vδ1-PD-L1 Bispecific Antibodies Enhances Blood Vδ1 Priming

As described herein, one advantageous property of the Vδ1-PD-L1 multispecific antibodies of the disclosure is that they can provoke peripheral proliferation and priming of Vδ1 γδ T cells via crosslinking to PD-L1+ normal immune cells, i.e. in a tumour free system. This example evidences how stimulation with Vδ1-PD-L1 bispecific antibodies primes Vδ1 cells in the blood by analysing key cell surface markers associated with activation.

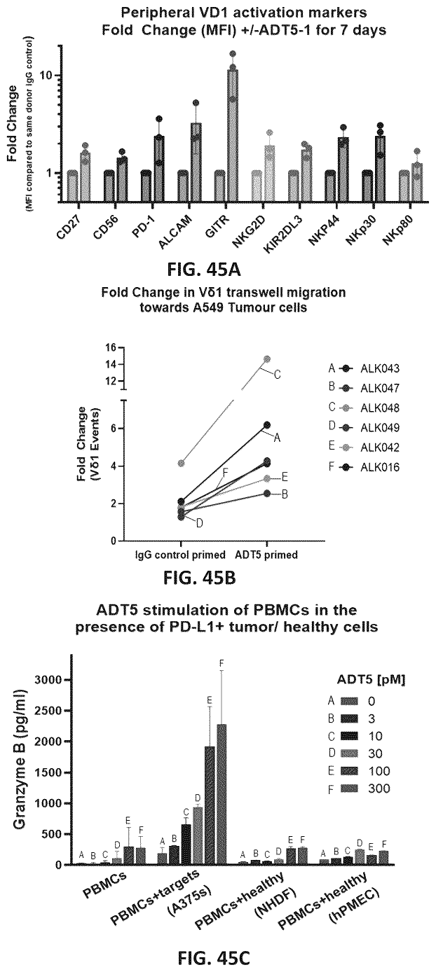

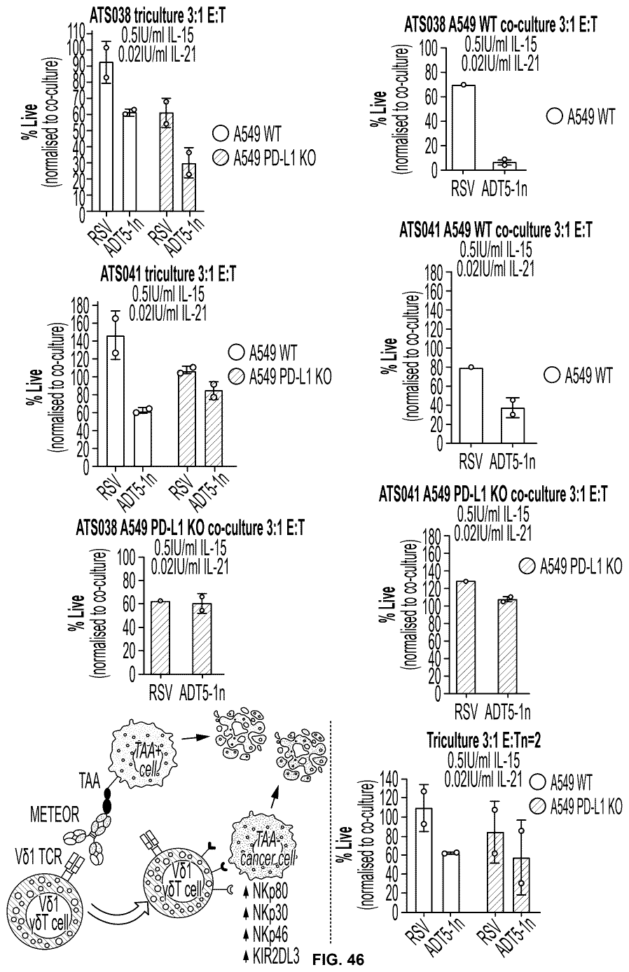

PBMCs from healthy donors were stimulated with 10 μM of anti-RSV IgG control or ADT1-4-2/ADT5-1 in basal growth media incubated at 37° C., 5% CO2 for 7 days. PBMCs were collected, and stained with fluorochrome-labeled anti-CD3, anti-Vδ1, anti-NKG2D, anti-NKp30, anti-NKp44, NKp80, KIR2DL3, anti-CD27, anti-CD56, anti-PD-1, anti-GITR and anti-ALCAM detection antibodies before assessing on a MACSquant® 16 Flow Cytometer (Miltenyi Biotec®) to determine the cell surface expression of relevant proteins on Vδ1 γδT cells. The fold increase in cell surface expression over the IgG control stimulated Vδ1γδT cells is shown in FIG. 45A.

Conclusions: This data demonstrates that Vδ1/PD-L1 bispecific antibodies enhance the cell surface expression of key cell surface markers associated with activation such as CD27, CD56, PD-1, ALCAM, GITR, NKG2D, KIR2DL3, NKp30, NKp44 and NKp80 on peripheral blood Vδ1 γδT cells.

### Example 21A. Differentiation of Peripheral Vδ1 γδ T Cell Phenotype into a Mixed Memory- & Effector-Like Phenotype

Another advantageous property of the Vδ1-PD-L1 multispecific antibodies of the disclosure is that they can activate peripheral Vδ1 γδ T cells into more mature, effector-like phenotypes as well as memory-like phenotypes. This is another example of priming Vδ1 γδ T cells with multispecific antibodies of the disclosure as it is expected to improve the anti-tumor functionality of the activated, primed Vδ1 γδ T cells.

Human γδ T cell phenotypes are typically characterised based on expression of CD27 and CD45RA:

| yδ T cell phenotype | Expression pattern |
|---|---|
| Naive | CD27⁺CD45RA⁺ |
| Central memory (CM) | CD27⁺CD45RA⁻ |
| Effector memory (EM) | CD27⁻CD45RA⁻ |
| Terminally differentiated effector memory (EMRA) | CD27⁻CD45RA⁺ |

Different phenotypes are associated with distinct roles in the immune response.

### Materials and Methods:

Human peripheral blood mononuclear cells (PBMCs) were prepared, counted, seeded, treated with antibodies of the disclosure and controls and then cultured as described in Example 19. Their phenotype was then assessed by flow cytometry before cells were harvested and stained, again as described in Example 19, using antibody clone REA499 for CD27 staining and antibody close H1100 for CD45RA staining. Flow cytometry was utilized (see Example 19 for detailed methods) to evaluate the effect of antibodies of the disclosure (TAK-505, ADT1-4-2/ADT5-1) and control test articles on differentiation of peripheral Vδ1 γδ T cell phenotype. Gating was performed by gating on live CD3+ pan-γδ TCR+Vδ1+ cells and then gating for CD27 CD45RA status to evaluate the activity of TAK-505 and control test articles on the frequency (%+) of CD27 and CD44RA in this population. The data analysis was performed as described in Example 19.

### Results:

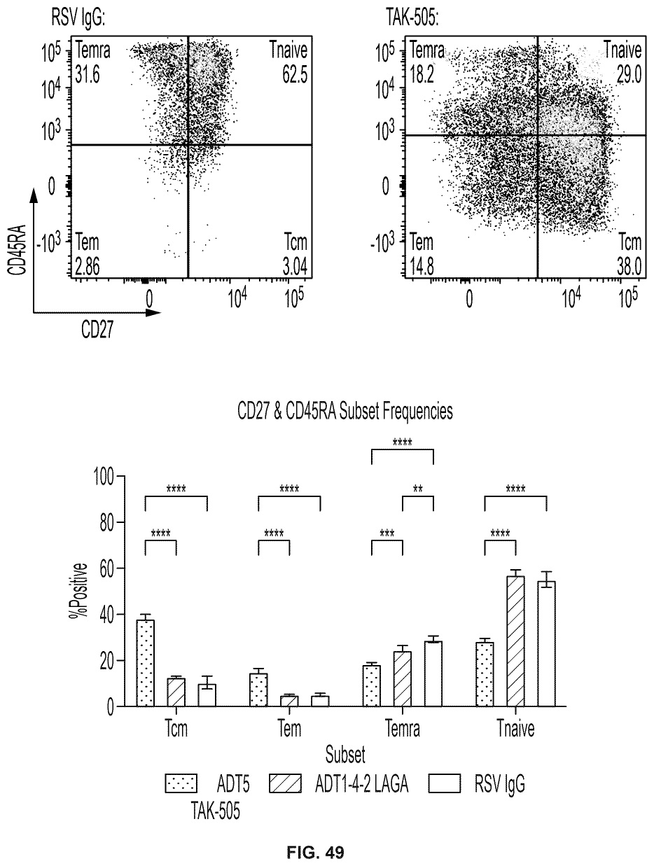

The results (see FIG. 49) demonstrate that PBMC populations treated with antibodies of the disclosure (TAK-505, ADT1-4-2/ADT5-1) had a greater percentage of central memory and effector memory T cell phenotypes than PBMC populations treated with control antibodies.

### Conclusions:

Antibodies of the disclosure (TAK-505, ADT1-4-2/ADT5-1) induce differentiation of peripheral Vδ1 γδ T cell phenotype into a mixed memory- & effector-like phenotype, particularly increasing central memory and effector memory phenotypes. Central memory T cells maintain immune memory for a long time and quickly mediate immune response after antigen stimulation. Effector memory T cells (situated mainly at the site of inflammation) execute rapid responses e.g. secrete cytokines and exert cytotoxicity. Treatment with antibodies of the disclosure can therefore be expected to improve the anti-tumor functionality of the activated, primed Vδ1 γδ T cells.

### Example 22. Stimulation with Vδ1-PD-L1 Bispecific Antibodies Enhances Blood Vδ1 Tumor Homing

Another benefit of the Vδ1-PD-L1 multispecific antibodies of the disclosure is that the peripheral Vδ1 γδ T cell proliferation also leads to upregulation of key homing receptors, promoting chemotaxis of the primed Vδ1 cells towards the tumour. This example explores how stimulation with Vδ1-PD-L1 bispecific antibodies enhances blood Vδ1 tumour homing.

PBMCs from healthy donors were stimulated with 10 μM of anti-RSV IgG control or ADT1-4-2/ADT5-1 for 7 days. A549 tumor cells were pre-seeded into 24 well plates and incubated at 37° C., 5% CO2 for 24 hours before day 7 PBMCs were collected, resuspended in basal growth media and seeded into the top chamber of 3.0 μm 24-well plate transwell inserts (Falcon®) containing A549 cells in the bottom chamber. Transwell inserts were incubated at 37° C., 5% CO2 for 24 hours before cells from the bottom and top chamber were collected and stained with fluorochrome-labeled anti-CD3, anti-Vδ1 and anti-CD45. The numbers of transwell migrated Vδ1 γδT cells were assessed on a MACSquant® 16 Flow Cytometer (Miltenyi Biotec®). The fold change in the number of Vδ1 γδT cell events in the bottom compared to the top chambers are shown in FIG. 45B.

Conclusions: This data demonstrates that Vδ1/PD-L1 bispecific antibodies enhance the chemotaxis of peripheral blood Vδ1 γδT cells towards tumor cells.

### Example 22A. Vδ1/PD-L1 Bispecific Antibodies Drive Immune Cell Infiltration and Normal Tissue Targeting Using Healthy and Tumor Organ-on-a-Chip Models

In this experiment, microphysiological organ-on-chip models, simulating human lung, colon, and stomach tissues, were utilised to demonstrate the tumour homing abilities of blood Vδ1 γδT cells primed with antibodies of the disclosure, as well as evaluate of on-target, off-tumor toxicity.

PBMCs were infused into a vascular channel on the chips and then allowed to migrate into a mesenchymal compartment and through into an epithelial cell/tumor cell compartment. The chips were run either as “tumor chip”, where a tumor cell line is implanted into the epithelial part of the chip, or they are run as a healthy chip where normal human epithelial cells from different anatomical locations are implanted into the epithelial area.

### Materials and Methods:

Mimetas® 3-Lane 40 OrganoPlates® were used to construct healthy and tumor chips. Each plate consists of 40 chips, with each chip composed of three microfluidic channels. The 3 channels were seeded with the key cellular components of 3 healthy tissues (stomach, colon, and lung) and 2 tumor tissues (gastric carcinoma and melanoma) as detailed in the below table.

Cellular Composition of Healthy and Tumor Chips

| | Top channel | Middle channel | Bottom channel |
|---|---|---|---|
| Chip Type | (Epithelium) | (ECM) | (Endothelium) |
| Healthy Stomach chip | Healthy gastric | Gastric fibroblasts | Umbilical vein |
| | epithelial cells | | endothelial-RFP |
| Healthy Colon chip | Healthy colonic | Colon fibroblasts | Umbilical vein |
| | epithelial cells | | endothelial-RFP |
| Healthy Lung chip | Healthy alveolar | Alveolar fibroblasts | Umbilical vein |
| | epithelial cells | | endothelial-RFP |
| Gastric cancer chip | Gastric cancer cells | Gastric fibroblasts | Umbilical vein |
| | (SNU-620) | | endothelial-RFP |
| Melanoma chip | Skin cancer cells (A375) | Dermal fibroblasts | Umbilical vein |
| | | | endothelial-RFP |

Formation of the ECM Barrier: The middle channel of each chip was utilized for the formation of the ECM physical barrier. To achieve this, 4 mg/mL rat tail collagen-I was prepared by mixing 5 mg/mL stock solution of rat tail collagen-I with 1N NaOH and 10×PBS. The collagen solution was then mixed with tissue-specific fibroblasts to reach a final density of 200,000 cells/mL and 2 μL of the gel solution containing fibroblasts was seeded into each chip. The OrganoPlate® was then placed in a humidified incubator (37° C., 5% CO₂) for 15 minutes to allow polymerization of the collagen gel. After polymerization, 30 μL of fibroblast media was added into the ECM middle channel inlet to hydrate the collagen hydrogel containing the fibroblasts before returning the plate to the incubator.

Formation of the 3D endothelial barrier: The bottom channel of each chip was utilized for the formation of the 3D endothelial barrier. To achieve this, the bottom channel was first coated by adding 40 μL of a 0.1% gelatin solution into the inlet of the bottom channel and incubating for 10 minutes at room temperature. The gelatin solution was then removed and replaced with 50 μL of endothelial medium. Umbilical vein endothelial-RFP (HUVECs-RFP) were trypsinized, resuspended in endothelial medium, and adjusted to a cell seeding density of 10×10⁶ cells/mL. The cell suspension was seeded in a 2 μL volume into the outlet of the bottom channel. The OrganoPlate® was placed at a 75-degree angle against the plate stand for 1 hour to allow cell attachment to the ECM. Once the cells attached, 50 μL of endothelial medium was added into the bottom channel outlet to support further growth of the tubular endothelialized channel.

Formation of 3D Epithelium: The top channel of each chip was utilized for the formation of the 3D epithelial barrier of both healthy and tumor chips as follows. Since SNU620 are suspension cells, they are seeded in a low gel concentration to maintain their presence in the top compartment. For consistency across tumor chips, A375 cells were cultured in gel matrix as well. To do this, 1 mg/mL rat tail collagen-I was prepared by mixing 5 mg/mL stock solution of rat tail collagen-I with 1 N NaOH and 10×PBS. Tumor cells (SNU-620 or A375) were trypsinized and resuspended in 1 mg/mL collagen-I gel mixture at a density of 10×10⁶ cells/mL. The collagen-cell mixture was then deposited in a 2 μL volume into the top channel inlet. The OrganoPlate® was placed in a humidified incubator (37° C., 5% CO2) for 15 minutes to allow polymerization of the collagen-I gel. After polymerization, 50 μL of RPMI 1640 medium was added into the top medium outlet to support tumor cell culture. For formation of heathy epithelium to establish the 3D tubular epithelial compartment, the top channel was first coated by adding 40 μL of a 0.1% gelatin solution into the inlet of the top channel and incubating for 10 minutes at room temperature. The gelatin solution was then removed and replaced with 50 μL of epithelial medium. Alveolar epithelial cells, gastric epithelial cells, or colon epithelial cells were thawed and resuspended in epithelial cell media at a density of 10×10⁶ cells/mL. The cell suspension was then seeded in a 2 μL volume into the outlet of the top channel. The OrganoPlate® was placed at a 75-degree angle against the plate stand for 2 hours to allow cell attachment to the ECM. Once the cells attached, 50 μL of epithelial medium was added into the top channel outlet to support further growth.

After 4 days of seeding into the OrganoPlate®, healthy and tumor chips formed a confluent culture. At this stage, the chips were ready for the addition of PBMCs, treatment with test and control articles, followed by the downstream assays including: PBMC extravasation and migration assay, assessment of barrier function, cytotoxicity assays and immunofluorescence staining of PD-L1 target expression and Vδ1 cells.

PBMCs Extravasation and Migration Assay: PBMCs were added to the endothelium compartment of both healthy and tumor chips to track their extravasation and migration across different channels in response to test and control articles. PBMCs were first labelled with CellTracker™ CMFDA dye and resuspended in SkinR medium at a concentration of 10,000 cells/μL. Next, the media was aspirated from the inlets and outlets of the top epithelial channel in all healthy and tumor chips. Fresh media was added in a 50 μL volume (epithelial media for healthy chips or RPMI 1640 for tumor chips) containing 300 ng/mL recombinant human CXCL12 (rhCXCL12) to the top inlet to establish baseline immune cell migration, followed by the addition of 50 μL of media (epithelial media for healthy chips or RPMI 1640 for tumor chips) to the top outlet to achieve a final concentration of 150 ng/ml of rhCXCL12 in the top epithelial channel of all healthy and tumor chips. Next, the bottom endothelial channel inlet and outlet were aspirated and replaced with 50 μL of endothelial medium, each containing the specified test article or control articles at a 1 nM concentration. This was followed by the addition of 50 μL of CMFDA-labeled PBMCs suspension in SkinR media to deliver 500,000 cells per chip and a 500 μM final concentration of test article or control articles in the bottom endothelialized channel. A concentration of 500 μM was selected to enable TAK 505-mediated priming of PBMCs in the endothelium over 7 days while also maximizing potential signals in healthy epithelium. The chips were imaged at Day 6 using a Zeiss® LSM900 confocal microscope to track the extravasation and migration of labeled PBMCs towards target healthy and tumor epithelium in response to test article or control articles. The images were then analyzed using Arivis® Pro 4.2.2 to calculate % Extravasation at target channel. % Extravasation at target channel=Number of PBMCs at target channel/total number of PBMCs

Where, Total number of PBMCs=(Bottom channel+ECM+Top channel).

Assessment of Barrier Function: Test and control articles were tested for their ability to affect the barrier functions of healthy epithelium and endothelium by measuring TEER. TEER of the OrganoPlate® was measured using an automated multichannel impedance spectrometer (OrganoTEER®, Mimetas® B, Oegstgeest, Netherlands) and 3-lane OrganoPlate®-fitting electrode boards. The plates were equilibrated at room temperature for 25 minutes before TEER measurement by putting them in the Biosafety cabinet to avoid any differences in temperature and flow. Point impedance TEER measurements were provided by the OrganoTEER® software in Ω·cm².

Cytotoxicity Assessment: First, the staining solution was prepared by mixing 20 μL ethidium homodimer-1 and 10 μL Hoechst 33342 in 10 mL DPBS. The media was aspirated from all chips, followed by 1 wash with DPBS for 5 minutes. The staining solution was then added, and the chips were incubated for 1 hour at 37° C. before imaging using a Zeiss® LSM900 confocal microscope. The images were then analyzed using Arivis® Pro 4.2.2. % Cytotoxicity was calculated as follows:

$$\% Cytotoxicity = \frac{Dead target cells in the top channel}{( Total Target cells in the top channel} \times 100 )$$

PD-L1 target expression was assessed by immunofluorescence in all healthy and tumor chips in both epithelial and endothelial compartments.

### Results:

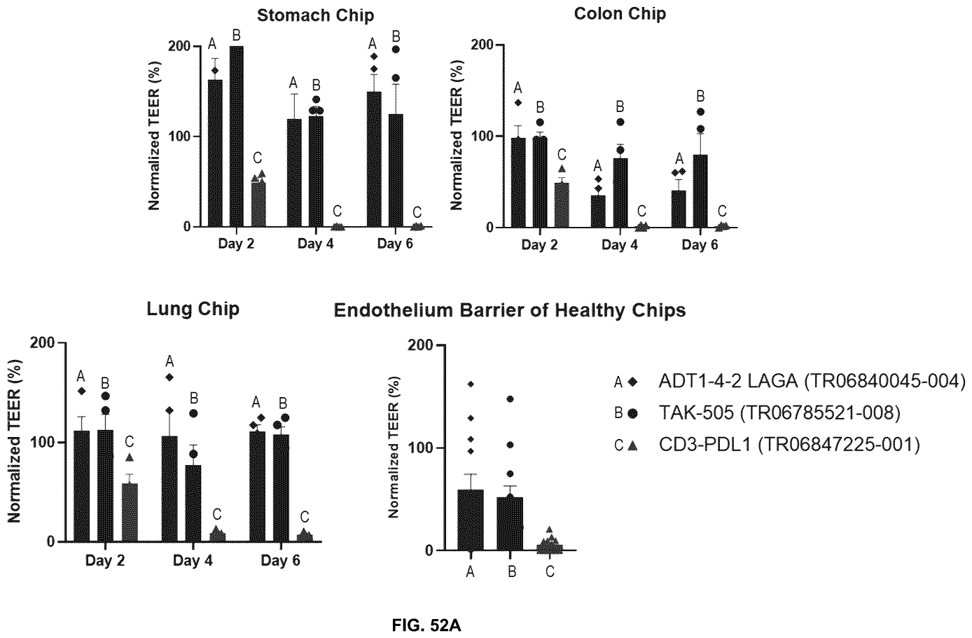

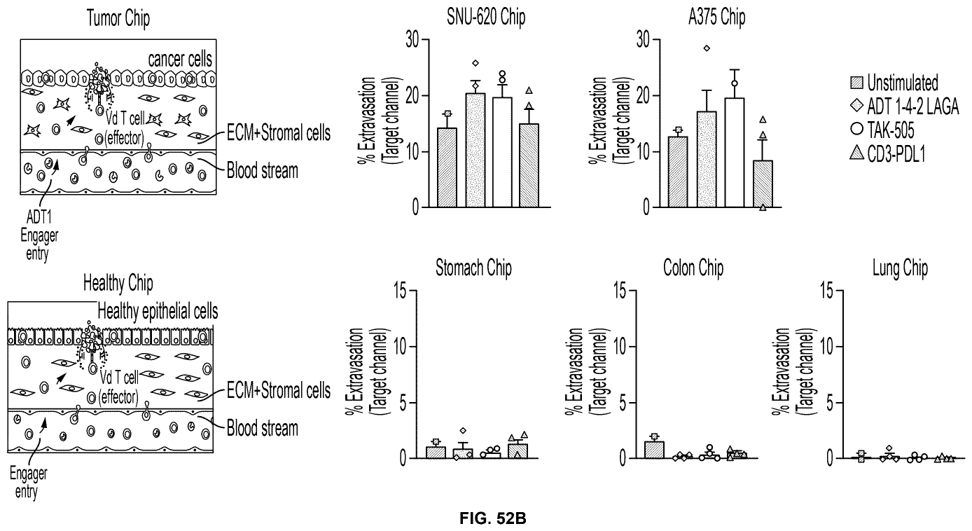

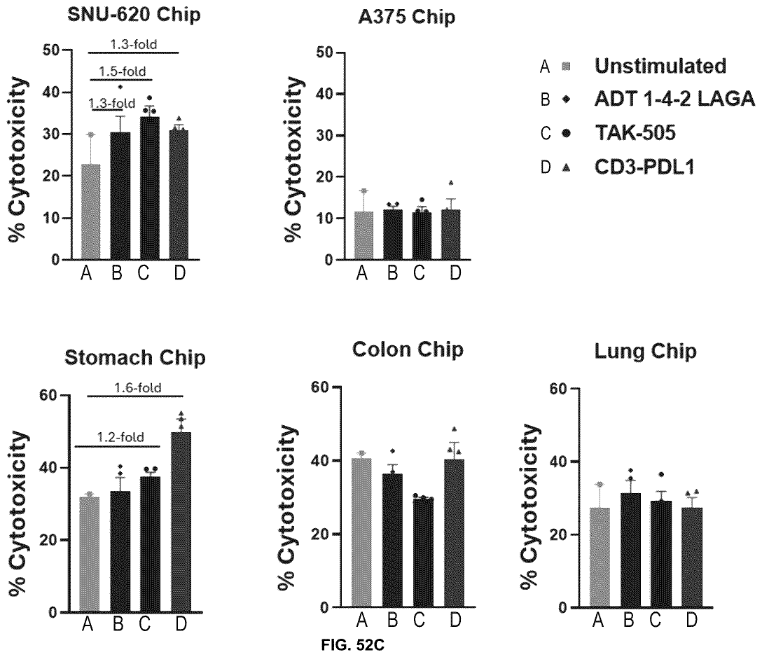

FIG. 52A demonstrates that antibodies of the disclosure (TAK-505, ADT1-4-2/ADT5-1) preserved the epithelial and endothelial barrier integrity of the chips when the transepithelial electrical resistance (TEER) was measured as a barrier function assessment at different time points. As shown, CD3-PDL1 treated chips lead to significant loss in the barrier function while antibodies of the disclosure preserved the epithelial and endothelial barrier integrity. Additionally, FIG. 52B demonstrates that antibodies of the disclosure (TAK-505, ADT1-4-2/ADT5-1) stimulates substantially increased migration of PBMCs to tumor epithelium relative to primary normal alveolar, colonic, and stomach epithelium, driving greater infiltration of PBMCs into the A375 tumor chips vs controls stimulated. The downstream effects of TAK-505 mediated immune cell infiltration on healthy epithelial and endothelial targeting was evaluated by barrier function and viability assessment. As shown in FIG. 52C, there were no findings of on-target, off-tumor toxicity in the 3D organ-on-chip model system. TAK-505 treatment had no effect on the viability of the endothelium or primary normal alveolar, colonic, and stomach epithelial cells. In contrast, the CD3/PD-L1 control engager resulted in a near complete loss of TEER in human umbilical vein endothelial cells and the 3 primary epithelial cell types.

### Conclusions:

Taken together, this highlights the ability of Vδ1/PD-L1 antibodies to drive migration of immune cells towards tumor sites, directing blood and tumor resident Vδ1 T cells to target PD-L1+ tumor cells. These data also demonstrate a favorable TAK-505 safety profile with no on-target, off-tumor toxicity and tumor specific migration in 3D organ-on-chip models of healthy lung, gastric, and colon tissues compared to the anti-CD3/PD-L1 control engager.

### Example 23. Bispecific Vδ1-Engagers Increase Innate Recognition of Tumor Cells

PBMCs from healthy donors were cultured alone or co-cultured tumor cells (A-375) or healthy cells (normal human dermal fibroblasts; NHDF, or hPMEC human pulmonary microvascular endothelial cells) at an E:T ratio of 50:1 in the presence of a titration of the Vδ1/PD-L1 bispecific ADT1-4-2/ADT5-1 and incubated 37° C., 5% CO2 for 72 hours. Supernatants were collected and Granzyme B levels quantified by single U-Plex MSD (Meso Scale Discovery®). Granzyme B secretion is presented as μg/ml in FIG. 45C.

In addition, FIG. 46 demonstrates that activation by bispecific Vδ1 engagers enhance innate recognition of TAA-tumor cells by Vδ1 γδ cells. FIG. 46 depicts coculture or triculture assays consisting of tissue-derived Vδ1 cells in culture with tumor cell targets, whereby Vδ1 cells are cultured with either PD-L1 positive tumor cells, PD-L1 negative tumor cells or a 50/50 mix of both PD-L1 positive and negative tumour cells. In all cases, cultures were setup with an overall Vδ1 effector cell to total tumor cell target ratio of 3:1 The results show that PD-L1 negative tumor targets are poorly targeted by Vδ1 cells in a Vδ1 positive PDL1 negative target coculture, however PD-L1 negative tumour cells are more readily targeted in a triculture system where Vδ1 cells are activated by PD-L1 positive tumor cells through bispecific Vδ1 engagers of the disclosure.

Conclusions: This data demonstrates that Vδ1/PD-L1 bispecific antibodies enhance Granzyme B secretion from peripheral blood Vδ1 γδT cells in the presence of PD-L1+ tumor cells but not in the presence of PD-L1+ healthy cells. Additionally the data demonstrates that activation by Vδ1/PD-L1 bispecific antibodies results in on tumour target-negative effects.

### Example 24. Alternative PD-L1 Multispecific Formats

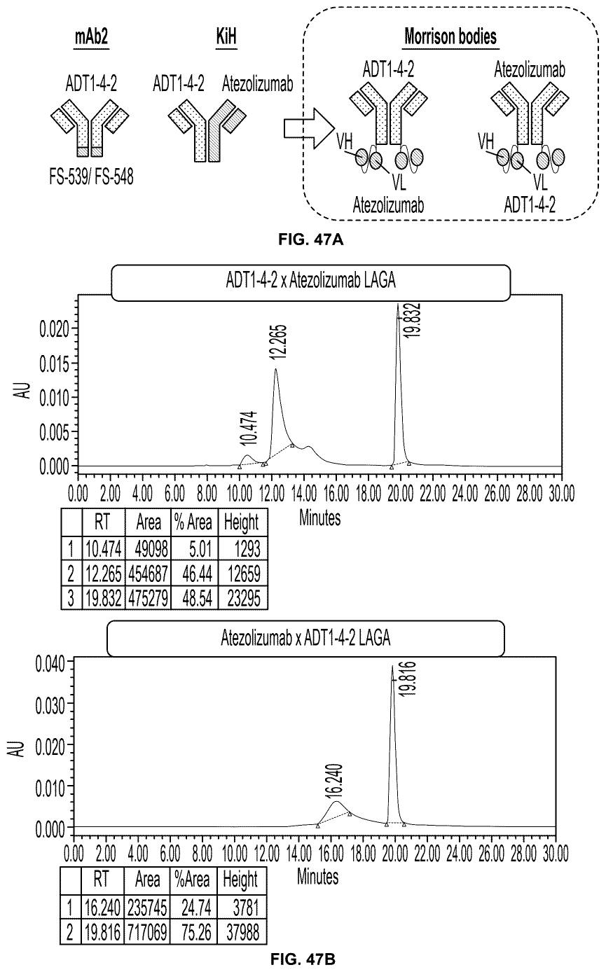

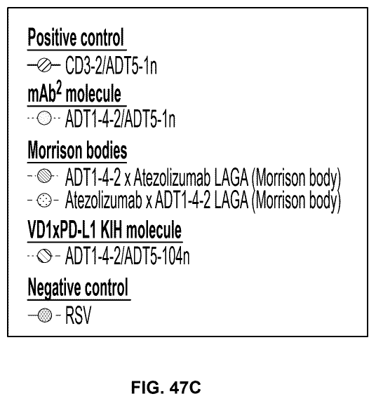

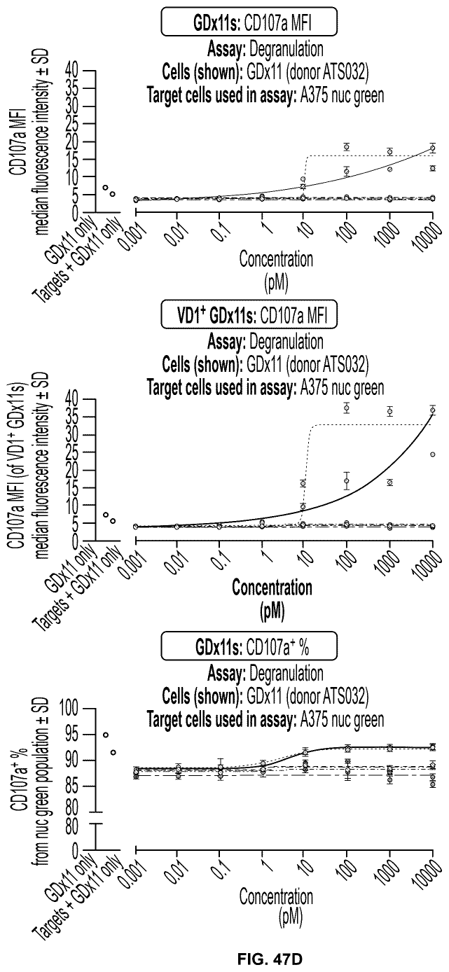

During the process of designing the presently claimed antibodies, various Vδ1×PD-L1 bispecific formats were designed and assayed for developability risks. As shown in FIG. 47A, Vδ1×PD-L1 mAb2 were compared to Vδ1×PD-L1 KiH (knob in hole) and Vδ1×PD-L1 Morrison body formats (with the anti-PD-L1 scFv hanging off the c-terminal of the heavy chain or the anti-Vδ1 scFv hanging off the c-terminal of the heavy chain).

The present mAb2 has previously been demonstrated to be superior to KiH in Vδ1 activation/cytotoxicity assays

HPLC analysis of the two Morrison body formats demonstrate both molecules were aggregated. They carry a sub-optimal profile (see FIG. 47B) as no single tall peak was observed (as would be expected from a molecule that is pure, stable and not degraded). The peak at 19 min can be disregarded as it was found to be L-arginine, present in the specific buffer used.

The bispecific formats were also tested for their ability to induce degranulation in GDx11 cells (degranulation assays used are as described elsewhere herein). FIG. 47C demonstrates that none of the Morrison bodies used in the experiment induces degranulation of GDx1 is, whereas VD1×PD-L1 mab2 and positive control (CD3×PD-L1 mab2) induce degranulation.

The bispecific formats were also tested for their ability to induce γδTCR down-regulation (γδTCR down-regulation assays used are as described elsewhere herein). The Morrison bodies were shown to down-regulate the γδTCR to a greater extent than the monovalent KiH formats (see FIG. 47D). However the γδTCR downregulation observed was not an indicator of downstream degranulation (See FIG. 47C]).

The bispecific formats were also tested for their ability to kill cells using a Cytotoxicity via OPERA® assay using GDx11 (donor ATS032) effector cells and A375 nuc green target cells at an E:T ratio of 1:1. When using a DRAQ7 dead cell nuclei count to assess potency (see FIG. 47D, it was shown that:
EC50: mab2 (0.5 nM)>Morrison bodies (1)>KIH (9.8)
Emax: mab2 (1075)>Morrison bodies (730)=KIH (727)

Results: Collectively, FIGS. 47A-47D demonstrate that Morrison bodies were consistently inferior to Mab2 in terms of activating effector cells towards a PD-L1 expressing cell line (A375). Morrison bodies either matched or were shown slightly more potent than the KIH antibody used for reference (but bivalency had already been established as important for full activation of Vδ1 γδ T cells). This shows that, not only can antibody structures be unpredictable, but an advantage of the presently claimed mAb2 Fcabs are that they help retain the IgG like format/structure and offer some degree of conditional activation via avidity.

### Example 25. Repriming Primed Vδ1 γδ T Cells with PD-L1+ Tumor Cells Amplifies Activation and Proliferation

It was found that antibodies of the disclosure (TAK-505, ADT1-4-2/ADT5-1) promotes additional activation and proliferation of primed Vδ1 γδ T cells that had been previously activated by antibodies of the disclosure when challenged with PD-L1⁺ tumor cells. This study is based on first priming PBMCs for 7 days with antibodies of the disclosure (TAK-505, ADT1-4-2/ADT5-1), and then depleting out the αβ T cells and re-culturing the sorted cells with PD-L1 tumor targets in the presence of more TAK-505.

### Materials and Methods:

Peripheral blood mononuclear cells (PBMCs) isolated from BioIVT leukopaks were isolated, cryopreserved, and thawed for culture in RPMI-1640 medium supplemented with heat-inactivated FBS and penicillin-streptomycin. In the priming phase, cells were treated with TAK-505 or control article (ADT1-4-2 LAGA) and incubated for 7 days, followed by αβ T cell depletion via MACS. In the re-priming phase, depleted PBMCs were then challenged with PD-L1⁺ tumour cells by co-culturing with A431 tumor cells (expressing eGFP and ffLuc), with or without 10 ng/ml IL-15 supplementation, and phenotyped using flow cytometry on days 10 and 14. Cultures were administered fresh media on day 10. Cultures were harvested on day 14 and analysed via cell counting as well as flow cytometry. Flow cytometry was performed using BD FACSymphony® A5 SORP and MACSQuant®16 instruments, with antibody panels targeting TCR Vδ1, PD-1, TOX, CD3, CD45RA, PD-L1, TIGIT, 4-1 BB, Ki67, CD27, and NKG2D. Compensation controls were prepared. FlowJo® analysis was used to quantify Vδ1 proliferation and activation markers, enabling dose-response and treatment effect readouts.

### Results:

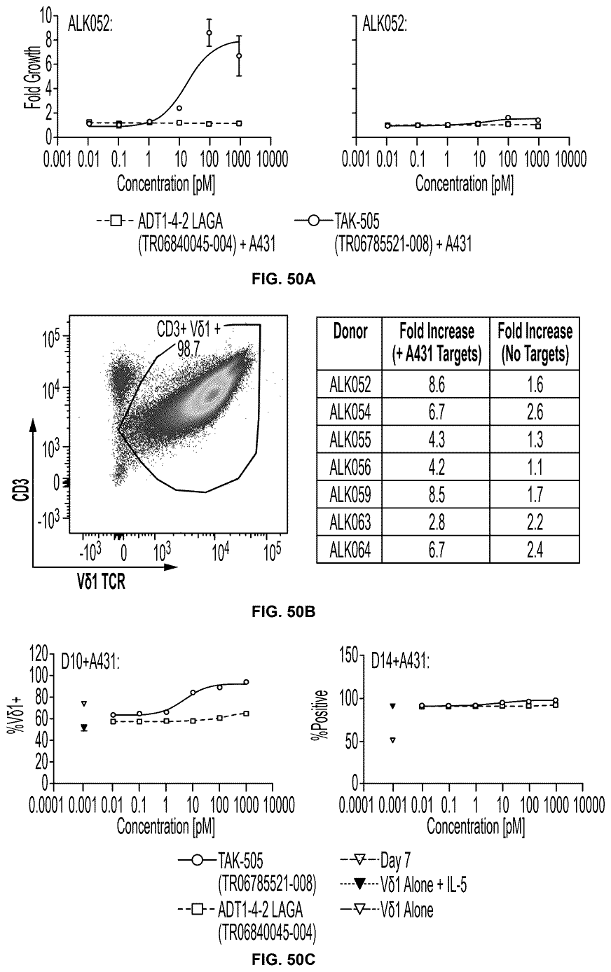

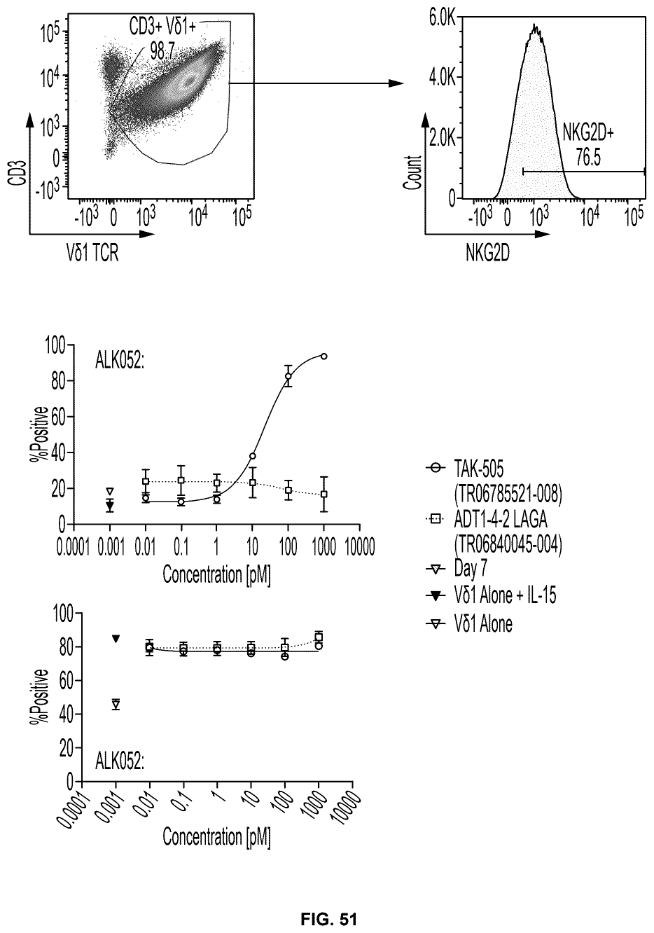

FIGS. 50A and 50B demonstrate that TAK-505 mediates 2.8-8.6-fold expansion of primed Vδ1 γδ T cells in the presence of A431 tumor cells and only minimal proliferation is observed in the absence of A431 tumor cells. FIG. 50C shows no Ki67 upregulation detected at day 14, with modest Ki67 upregulation at day 10 in some donors. FIG. 51 showcases that as the previously primed cells are exposed to tumor challenge (A431 tumor cells), stimulation with TAK-505 drives upregulation of NKG2D in a dose-dependent manner relative to the control molecule, and NKG2D expression does not change in the absence of A431 tumor cells.

### Conclusions:

Cells that have been primed with antibodies of the disclosure (TAK-505, ADT1-4-2/ADT5-1) are able to go on to proliferate again after priming when they encounter tumor threats in the presence of TAK-505. TAK-505 also helps to maintain expression of NKG2D when these Vδ1 cells are being rechallenged with PD-L1 tumor cells. TAK-505's ability to enhance NKG2D expression in this context indicates its ability to improve Vδ1 anti-tumor functionality after priming and during tumor encounter.

### EMBODIMENTS

The present disclosure includes at least the following numbered embodiments:
1. A multispecific antibody or antigen-binding fragment thereof comprising a Fab region and an Fc region, wherein the Fab region comprises a binding site specific for the variable delta 1 (Vδ1) chain of a γδ T cell receptor (TCR); and the Fc region comprises a binding site specific for PD-L1.
2. The multispecific antibody or antigen-binding fragment thereof of embodiment 1, wherein the PD-L1 binding site is located in a CH3 domain, wherein the PD-L1 binding site comprises:
   a. a first sequence located in the AB structural loop at positions 359 to 362 of the CH3 domain, wherein the PD-L1 binding site comprises an amino acid deletion at residue 358;
   b. optionally a second sequence located in the CD structural loop at positions 386 to 391; and
   c. a third sequence located in the EF structural loop at positions 413 to 421;
      and wherein the amino acid residue numbering is according to the EU numbering scheme.
3. The multispecific antibody or antigen-binding fragment thereof of embodiment 2, wherein:
   a. The first sequence has the sequence set forth in SEQ ID NO: 565, the second sequence has the sequence set forth in SEQ ID NO: 566, and the third sequence has the sequence set forth in SEQ ID NO: 567, and the residue at position 433 of the CH3 domain is arginine (R);
   b. The first sequence has the sequence set forth in SEQ ID NO: 569, the second sequence has the sequence set forth in SEQ ID NO: 566, and the third sequence has the sequence set forth in SEQ ID NO: 567;
   c. The first sequence has the sequence set forth in SEQ ID NO: 571, the second sequence has the sequence set forth in SEQ ID NO: 566, and the third sequence has the sequence set forth in SEQ ID NO: 572, and the sequence comprises an amino acid deletion at position 422;
   d. The first sequence has the sequence set forth in SEQ ID NO: 565, the second sequence has the sequence set forth in SEQ ID NO: 566, and the third sequence has the sequence set forth in SEQ ID NO: 572;
   e. The first sequence has the sequence set forth in SEQ ID NO: 565, the second sequence has the sequence set forth in SEQ ID NO: 566, and the third sequence has the sequence set forth in SEQ ID NO: 575;
   f. The first sequence has the sequence set forth in SEQ ID NO: 569, the second sequence has the sequence set forth in SEQ ID NO: 566, and the third sequence has the sequence set forth in SEQ ID NO: 572;
   g. The first sequence has the sequence set forth in SEQ ID NO: 578, the second sequence has the sequence set forth in SEQ ID NO: 566, and the third sequence has the sequence set forth in SEQ ID NO: 572;
   h. The first sequence has the sequence set forth in SEQ ID NO: 571, the second sequence has the sequence set forth in SEQ ID NO: 566, and the third sequence has the sequence set forth in SEQ ID NO: 580;
   i. The first sequence has the sequence set forth in SEQ ID NO: 571, the second sequence has the sequence set forth in SEQ ID NO: 566, and the third sequence has the sequence set forth in SEQ ID NO: 582;
   j. The first sequence has the sequence set forth in SEQ ID NO: 571, the second sequence has the sequence set forth in SEQ ID NO: 566, and the third sequence has the sequence set forth in SEQ ID NO: 584.
4. The multispecific antibody or antigen-binding fragment thereof according to embodiment 1, wherein the CH3 domain is a human IgG CH3 domain engineered to comprise the PD-L1 binding site.
5. The multispecific antibody or antigen-binding fragment thereof according to embodiment 1, wherein the Fc region is a human IgG Fc region comprising the CH3 domain engineered to comprise the PD-L1 binding site.
6. The multispecific antibody or antigen-binding fragment thereof according to any preceding embodiment, wherein the amino acid sequence of the CH3 domain is at least 90% identical to, or at least 95% identical to, or 100% identical to:
   a. SEQ ID NO: 564
   b. SEQ ID NO: 568;
   c. SEQ ID NO: 570;
   d. SEQ ID NO: 573;
   e. SEQ ID NO: 574;
   f. SEQ ID NO: 576;
   g. SEQ ID NO: 577;
   h. SEQ ID NO: 579;
   i. SEQ ID NO: 581;
   j. SEQ ID NO: 583.
7. The multispecific antibody or antigen-binding fragment thereof of any previous embodiment, wherein the antibody is Fc enabled.
8. The multispecific antibody or antigen-binding fragment thereof of any previous embodiment, wherein the heavy chain constant domain comprises:
   a. SEQ ID NO: 585 or SEQ ID NO: 586;
   b. SEQ ID NO: 595 or SEQ ID NO: 596;
   c. SEQ ID NO: 605 or SEQ ID NO: 606;
   d. SEQ ID NO: 615 or SEQ ID NO: 616;
   e. SEQ ID NO: 625 or SEQ ID NO: 626;
   f. SEQ ID NO: 635 or SEQ ID NO: 636;
   g. SEQ ID NO: 645 or SEQ ID NO: 646;
   h. SEQ ID NO: 655 or SEQ ID NO: 656;
   i. SEQ ID NO: 665 or SEQ ID NO: 666; or
   j. SEQ ID NO: 675 or SEQ ID NO: 676.
9. The multispecific antibody of any previous embodiment, comprising:
   a. SEQ ID NO: 589; or SEQ ID NO: 414 and SEQ ID NO: 587;
   b. SEQ ID NO: 590; or SEQ ID NO: 414 and SEQ ID NO: 588;
   c. SEQ ID NO: 599; or SEQ ID NO: 414 and SEQ ID NO: 597;
   d. SEQ ID NO: 600; or SEQ ID NO: 414 and SEQ ID NO: 598;
   e. SEQ ID NO: 609; or SEQ ID NO: 414 and SEQ ID NO: 607;
   f. SEQ ID NO: 610; or SEQ ID NO: 414 and SEQ ID NO: 608;
   g. SEQ ID NO: 619; or SEQ ID NO: 414 and SEQ ID NO: 617;
   h. SEQ ID NO: 620; or SEQ ID NO: 414 and SEQ ID NO: 618;
   i. SEQ ID NO: 629; or SEQ ID NO: 414 and SEQ ID NO: 627;
   j. SEQ ID NO: 630; or SEQ ID NO: 414 and SEQ ID NO: 628;
   k. SEQ ID NO: 639; or SEQ ID NO: 414 and SEQ ID NO: 637;
   l. SEQ ID NO: 640; or SEQ ID NO: 414 and SEQ ID NO: 638;
   m. SEQ ID NO: 649; or SEQ ID NO: 414 and SEQ ID NO: 647;
   n. SEQ ID NO: 650; or SEQ ID NO: 414 and SEQ ID NO: 648;
   o. SEQ ID NO: 659; or SEQ ID NO: 414 and SEQ ID NO: 657;
   p. SEQ ID NO: 660; or SEQ ID NO: 414 and SEQ ID NO: 658;
   q. SEQ ID NO: 669; or SEQ ID NO: 414 and SEQ ID NO: 667;
   r. SEQ ID NO: 670; or SEQ ID NO: 414 and SEQ ID NO: 668;
   s. SEQ ID NO: 679; or SEQ ID NO: 414 and SEQ ID NO: 677; or
   t. SEQ ID NO: 680; or SEQ ID NO: 414 and SEQ ID NO: 678.
10. The multispecific antibody or antigen-binding fragment thereof of any previous embodiment, wherein the Fc region thereof binds to human PD-L1 with a binding affinity (K_{D}, for example as measured by surface plasmon resonance) of less than about 150 nM.
11. The multispecific antibody or antigen-binding fragment thereof of any previous embodiment, wherein the Fc region thereof binds to human PD-L1 with a binding affinity (K_{D}, for example as measured by surface plasmon resonance) of less than about 20 nM.
12. The multispecific antibody or antigen-binding fragment thereof of any previous embodiment, wherein the Fc region thereof binds to human PD-L1 with a binding affinity (K_{D}, for example as measured by surface plasmon resonance) of less than about 10 nM.
13. The multispecific antibody or antigen-binding fragment thereof of any previous embodiment, wherein the Fc region thereof binds to human PD-L1 with a binding affinity (K_{D}, for example as measured by surface plasmon resonance) of less than about 5 nM.
14. The multispecific antibody or antigen-binding fragment thereof of any previous embodiment, wherein the Fc region thereof binds to PD-L1 with a binding affinity (K_{D}, for example as measured by surface plasmon resonance) of less than about 4 nM.
15. The multispecific antibody or antigen-binding fragment thereof of any previous embodiment, wherein the Fab region comprises:
   a) a heavy chain variable region comprising a VHCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 55 to 78 and 133 to 143; and/or
   b) a light chain variable region comprising a VLCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 82 to 105 and 147 to 157.
16. The multispecific antibody or antigen-binding fragment thereof of embodiment 15, wherein the Fab region comprises:
   a heavy chain variable region comprising:
      a VHCDR1 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NOs: 51, 52 and 130;
      a VHCDR2 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NO: 53 and 131; and
      a VHCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NOs: 55 to 78 and 133 to 143; and/or
   a light chain variable region comprising:
      a VLCDR1 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NO: 79 and 144;
      a VLCDR2 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NO: 80 and 145; and
      a VLCDR3 comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of: SEQ ID NOs: 82 to 105 and 147 to 157.
17. The multispecific antibody or antigen-binding fragment thereof of embodiment 15 or 16, wherein the Fab region comprises:
   a heavy chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 2 to 25 and 107 to 117; and/or
   a light chain variable region comprising or consisting of a sequence having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to a sequence selected from the group consisting of SEQ ID NOs: 27 to 50 and 119 to 129.
18. The multispecific antibody or antigen-binding fragment thereof of one of embodiments 1 to 10, wherein the Fab region comprises:
   a. a VHCDR3 comprising or consisting of an amino acid sequence having at least 90% sequence identity to any one of SEQ ID NOs: 55 to 78; and/or
   b. a VLCDR3 comprising or consisting of an amino acid sequence having at least 90% sequence identity to any one of SEQ ID NOs: 82 to 105.
19. The multispecific antibody or antigen-binding fragment thereof of any previous embodiment, wherein the Fab region comprises:
   a. a VHCDR3 comprising or consisting of the amino acid sequence of any one of SEQ ID NOs: 55 to 78; and/or
   b. a VLCDR3 comprising or consisting of the amino acid sequence of any one of SEQ ID NOs: 82 to 105
20. The multispecific antibody or antigen-binding fragment thereof of any previous embodiment, wherein the Fab region comprises:
   a. a VHCDR1 comprising or consisting of an amino acid sequence having at least 90% sequence identity to SEQ ID NO: 51 or SEQ ID NO: 52;
   b. a VHCDR2 comprising or consisting of an amino acid sequence having at least 90% sequence identity to SEQ ID NO: 53;
   c. a VLCDR1 comprising or consisting of an amino acid sequence having at least 90% sequence identity to SEQ ID NO: 79; and/or d. a VLCDR2 comprising or consisting of an amino acid sequence having at least 90% sequence identity to SEQ ID NO: 80.
21. The multispecific antibody or antigen-binding fragment thereof of any previous embodiment, wherein the Fab region comprises:
   a. a VHCDR1 comprising or consisting of the amino acid sequence of SEQ ID NO: 51 or SEQ ID NO: 52;
   b. a VHCDR2 comprising or consisting of the amino acid sequence of SEQ ID NO: 53;
   c. a VLCDR1 comprising or consisting of the amino acid sequence of SEQ ID NO: 79; and/or
   d. a VLCDR2 comprising or consisting of the amino acid sequence of SEQ ID NO: 80.
22. The multispecific antibody or antigen-binding fragment thereof of any previous embodiment, wherein the Fab region comprises:
   a. a VHCDR1, a VHCDR2 and a VHCDR3 comprising or consisting of the amino acid sequences of SEQ ID NO: 51, 53 and 68, respectively, and a VLCDR1, a VLCDR2 and a VLCDR3 comprising or consisting of the amino acid sequences of SEQ ID NOs: 79, 80 and 95, respectively;
   b. a VHCDR1, a VHCDR2 and a VHCDR3 comprising or consisting of the amino acid sequences of SEQ ID NO: 51, 53 and 55, respectively, and a VLCDR1, a VLCDR2 and a VLCDR3 comprising or consisting of the amino acid sequences of SEQ ID NOs: 79, 80 and 82, respectively;
   c. a VHCDR1, a VHCDR2 and a VHCDR3 comprising or consisting of the amino acid sequences of SEQ ID NO: 51, 53 and 56, respectively, and a VLCDR1, a VLCDR2 and a VLCDR3 comprising or consisting of the amino acid sequences of SEQ ID NOs: 79, 80 and 83, respectively;
   d. a VHCDR1, a VHCDR2 and a VHCDR3 comprising or consisting of the amino acid sequences of SEQ ID NO: 51, 53 and 57, respectively, and a VLCDR1, a VLCDR2 and a VLCDR3 comprising or consisting of the amino acid sequences of SEQ ID NOs: 79, 80 and 84, respectively;
   e. a VHCDR1, a VHCDR2 and a VHCDR3 comprising or consisting of the amino acid sequences of SEQ ID NO: 51, 53 and 58, respectively, and a VLCDR1, a VLCDR2 and a VLCDR3 comprising or consisting of the amino acid sequences of SEQ ID NOs: 79, 80 and 85, respectively;
   f. a VHCDR1, a VHCDR2 and a VHCDR3 comprising or consisting of the amino acid sequences of SEQ ID NO: 51, 53 and 59, respectively, and a VLCDR1, a VLCDR2 and a VLCDR3 comprising or consisting of the amino acid sequences of SEQ ID NOs: 79, 80 and 86, respectively;
   g. a VHCDR1, a VHCDR2 and a VHCDR3 comprising or consisting of the amino acid sequences of SEQ ID NO: 51, 53 and 60, respectively, and a VLCDR1, a VLCDR2 and a VLCDR3 comprising or consisting of the amino acid sequences of SEQ ID NOs: 79, 80 and 87, respectively;
   h. a VHCDR1, a VHCDR2 and a VHCDR3 comprising or consisting of the amino acid sequences of SEQ ID NO: 52, 53 and 61, respectively, and a VLCDR1, a VLCDR2 and a VLCDR3 comprising or consisting of the amino acid sequences of SEQ ID NOs: 79, 80 and 88, respectively;
   i. a VHCDR1, a VHCDR2 and a VHCDR3 comprising or consisting of the amino acid sequences of SEQ ID NO: 51, 53 and 62, respectively, and a VLCDR1, a VLCDR2 and a VLCDR3 comprising or consisting of the amino acid sequences of SEQ ID NOs: 79, 80 and 89, respectively;
   j. a VHCDR1, a VHCDR2 and a VHCDR3 comprising or consisting of the amino acid sequences of SEQ ID NO: 51, 53 and 63, respectively, and a VLCDR1, a VLCDR2 and a VLCDR3 comprising or consisting of the amino acid sequences of SEQ ID NOs: 79, 80 and 90, respectively;
   k. a VHCDR1, a VHCDR2 and a VHCDR3 comprising or consisting of the amino acid sequences of SEQ ID NO: 51, 53 and 64, respectively, and a VLCDR1, a VLCDR2 and a VLCDR3 comprising or consisting of the amino acid sequences of SEQ ID NOs: 79, 80 and 91, respectively;
   l. a VHCDR1, a VHCDR2 and a VHCDR3 comprising or consisting of the amino acid sequences of SEQ ID NO: 51, 53 and 65, respectively, and a VLCDR1, a VLCDR2 and a VLCDR3 comprising or consisting of the amino acid sequences of SEQ ID NOs: 79, 80 and 92, respectively;
   m. a VHCDR1, a VHCDR2 and a VHCDR3 comprising or consisting of the amino acid sequences of SEQ ID NO: 52, 53 and 66, respectively, and a VLCDR1, a VLCDR2 and a VLCDR3 comprising or consisting of the amino acid sequences of SEQ ID NOs: 79, 80 and 93, respectively;
   n. a VHCDR1, a VHCDR2 and a VHCDR3 comprising or consisting of the amino acid sequences of SEQ ID NO: 51, 53 and 67, respectively, and a VLCDR1, a VLCDR2 and a VLCDR3 comprising or consisting of the amino acid sequences of SEQ ID NOs: 79, 80 and 94, respectively;
   o. a VHCDR1, a VHCDR2 and a VHCDR3 comprising or consisting of the amino acid sequences of SEQ ID NO: 51, 53 and 68, respectively, and a VLCDR1, a VLCDR2 and a VLCDR3 comprising or consisting of the amino acid sequences of SEQ ID NOs: 79, 80 and 95, respectively;
   p. a VHCDR1, a VHCDR2 and a VHCDR3 comprising or consisting of the amino acid sequences of SEQ ID NO: 51, 53 and 69, respectively, and a VLCDR1, a VLCDR2 and a VLCDR3 comprising or consisting of the amino acid sequences of SEQ ID NOs: 79, 80 and 96, respectively;
   q. a VHCDR1, a VHCDR2 and a VHCDR3 comprising or consisting of the amino acid sequences of SEQ ID NO: 51, 53 and 70, respectively, and a VLCDR1, a VLCDR2 and a VLCDR3 comprising or consisting of the amino acid sequences of SEQ ID NOs: 79, 80 and 97, respectively;
   r. a VHCDR1, a VHCDR2 and a VHCDR3 comprising or consisting of the amino acid sequences of SEQ ID NO: 51, 53 and 71, respectively, and a VLCDR1, a VLCDR2 and a VLCDR3 comprising or consisting of the amino acid sequences of SEQ ID NOs: 79, 80 and 98, respectively;
   s. a VHCDR1, a VHCDR2 and a VHCDR3 comprising or consisting of the amino acid sequences of SEQ ID NO: 51, 53 and 72, respectively, and a VLCDR1, a VLCDR2 and a VLCDR3 comprising or consisting of the amino acid sequences of SEQ ID NOs: 79, 80 and 99, respectively;
   t. a VHCDR1, a VHCDR2 and a VHCDR3 comprising or consisting of the amino acid sequences of SEQ ID NO: 51, 53 and 73, respectively, and a VLCDR1, a VLCDR2 and a VLCDR3 comprising or consisting of the amino acid sequences of SEQ ID NOs: 79, 80 and 100, respectively;
   u. a VHCDR1, a VHCDR2 and a VHCDR3 comprising or consisting of the amino acid sequences of SEQ ID NO: 52, 53 and 74, respectively, and a VLCDR1, a VLCDR2 and a VLCDR3 comprising or consisting of the amino acid sequences of SEQ ID NOs: 79, 80 and 101, respectively;
   v. a VHCDR1, a VHCDR2 and a VHCDR3 comprising or consisting of the amino acid sequences of SEQ ID NO: 51, 53 and 75, respectively, and a VLCDR1, a VLCDR2 and a VLCDR3 comprising or consisting of the amino acid sequences of SEQ ID NOs: 79, 80 and 102, respectively;
   w. a VHCDR1, a VHCDR2 and a VHCDR3 comprising or consisting of the amino acid sequences of SEQ ID NO: 51, 53 and 76, respectively, and a VLCDR1, a VLCDR2 and a VLCDR3 comprising or consisting of the amino acid sequences of SEQ ID NOs: 79, 80 and 103, respectively;
   x. a VHCDR1, a VHCDR2 and a VHCDR3 comprising or consisting of the amino acid sequences of SEQ ID NO: 51, 53 and 77, respectively, and a VLCDR1, a VLCDR2 and a VLCDR3 comprising or consisting of the amino acid sequences of SEQ ID NOs: 79, 80 and 104, respectively; or
   y. a VHCDR1, a VHCDR2 and a VHCDR3 comprising or consisting of the amino acid sequences of SEQ ID NO: 51, 53 and 78, respectively, and a VLCDR1, a VLCDR2 and a VLCDR3 comprising or consisting of the amino acid sequences of SEQ ID NOs: 79, 80 and 105, respectively.
23. The multispecific antibody or antigen-binding fragment thereof of any previous embodiment, wherein the Fab region comprises
   a. a VH comprising an amino acid sequence having at least 90% sequence identity to any one of SEQ ID NOs: 2 to 25; and/or
   b. a VL comprising or consisting of an amino acid sequence having at least 90% identity to any one of SEQ ID NOs: 27 to 50.
24. The multispecific antibody or antigen-binding fragment thereof of any previous embodiment, wherein the Fab region comprises:
   a. a VH comprising or consisting of the amino acid sequence of any one of SEQ ID NOs: 2 to 25; and/or
   b. a VL comprising or consisting of the amino acid sequence of any one of SEQ ID NOs: 27 to 50.
25. The multispecific antibody or antigen-binding fragment thereof of any previous embodiment, wherein the Fab region comprises:
   a. a VH comprising or consisting of an amino acid sequence having at least 90% sequence identity to SEQ ID NO: 15 and a VL comprising or consisting of an amino acid sequence having at least 90% sequence identity to SEQ ID NO: 40;
   b. a VH comprising or consisting of an amino acid sequence having at least 90% sequence identity to SEQ ID NO: 2 and a VL comprising or consisting of an amino acid sequence having at least 90% sequence identity to SEQ ID NO: 27;
   c. a VH comprising or consisting of an amino acid sequence having at least 90% sequence identity to SEQ ID NO: 3 and a VL comprising or consisting of an amino acid sequence having at least 90% sequence identity to SEQ ID NO: 28;
   d. a VH comprising or consisting of an amino acid sequence having at least 90% sequence identity to SEQ ID NO: 4 and a VL comprising or consisting of an amino acid sequence having at least 90% sequence identity to SEQ ID NO: 29;
   e. a VH comprising or consisting of an amino acid sequence having at least 90% sequence identity to SEQ ID NO: 5 and a VL comprising or consisting of an amino acid sequence having at least 90% sequence identity to SEQ ID NO: 30;
   f. a VH comprising or consisting of an amino acid sequence having at least 90% sequence identity to SEQ ID NO: 6 and a VL comprising or consisting of an amino acid sequence having at least 90% sequence identity to SEQ ID NO: 31;
   g. a VH comprising or consisting of an amino acid sequence having at least 90% sequence identity to SEQ ID NO: 7 and a VL comprising or consisting of an amino acid sequence having at least 90% sequence identity to SEQ ID NO: 32;
   h. a VH comprising or consisting of an amino acid sequence having at least 90% sequence identity to SEQ ID NO: 8 and a VL comprising or consisting of an amino acid sequence having at least 90% sequence identity to SEQ ID NO: 33;
   i. a VH comprising or consisting of an amino acid sequence having at least 90% sequence identity to SEQ ID NO: 9 and a VL comprising or consisting of an amino acid sequence having at least 90% sequence identity to SEQ ID NO: 34;
   j. a VH comprising or consisting of an amino acid sequence having at least 90% sequence identity to SEQ ID NO: 10 and a VL comprising or consisting of an amino acid sequence having at least 90% sequence identity to SEQ ID NO: 35;
   k. a VH comprising or consisting of an amino acid sequence having at least 90% sequence identity to SEQ ID NO: 11 and a VL comprising or consisting of an amino acid sequence having at least 90% sequence identity to SEQ ID NO: 36;
   l. a VH comprising or consisting of an amino acid sequence having at least 90% sequence identity to SEQ ID NO: 12 and a VL comprising or consisting of an amino acid sequence having at least 90% sequence identity to SEQ ID NO: 37;
   m. a VH comprising or consisting of an amino acid sequence having at least 90% sequence identity to SEQ ID NO: 13 and a VL comprising or consisting of an amino acid sequence having at least 90% sequence identity to SEQ ID NO: 38;
   n. a VH comprising or consisting of an amino acid sequence having at least 90% sequence identity to SEQ ID NO: 14 and a VL comprising or consisting of an amino acid sequence having at least 90% sequence identity to SEQ ID NO: 39;
   o. a VH comprising or consisting of an amino acid sequence having at least 90% sequence identity to SEQ ID NO: 15 and a VL comprising or consisting of an amino acid sequence having at least 90% sequence identity to SEQ ID NO: 40;
   p. a VH comprising or consisting of an amino acid sequence having at least 90% sequence identity to SEQ ID NO: 16 and a VL comprising or consisting of an amino acid sequence having at least 90% sequence identity to SEQ ID NO: 41;
   q. a VH comprising or consisting of an amino acid sequence having at least 90% sequence identity to SEQ ID NO: 17 and a VL comprising or consisting of an amino acid sequence having at least 90% sequence identity to SEQ ID NO: 42;
   r. a VH comprising or consisting of an amino acid sequence having at least 90% sequence identity to SEQ ID NO: 18 and a VL comprising or consisting of an amino acid sequence having at least 90% sequence identity to SEQ ID NO: 43;
   s. a VH comprising or consisting of an amino acid sequence having at least 90% sequence identity to SEQ ID NO: 19 and a VL comprising or consisting of an amino acid sequence having at least 90% sequence identity to SEQ ID NO: 44;
   t. a VH comprising or consisting of an amino acid sequence having at least 90% sequence identity to SEQ ID NO: 20 and a VL comprising or consisting of an amino acid sequence having at least 90% sequence identity to SEQ ID NO: 45;
   u. a VH comprising or consisting of an amino acid sequence having at least 90% sequence identity to SEQ ID NO: 21 and a VL comprising or consisting of an amino acid sequence having at least 90% sequence identity to SEQ ID NO: 46;
   v. a VH comprising or consisting of an amino acid sequence having at least 90% sequence identity to SEQ ID NO: 22 and a VL comprising or consisting of an amino acid sequence having at least 90% sequence identity to SEQ ID NO: 47;
   w. a VH comprising or consisting of an amino acid sequence having at least 90% sequence identity to SEQ ID NO: 23 and a VL comprising or consisting of an amino acid sequence having at least 90% sequence identity to SEQ ID NO: 48;
   x. a VH comprising or consisting of an amino acid sequence having at least 90% sequence identity to SEQ ID NO: 24 and a VL comprising or consisting of an amino acid sequence having at least 90% sequence identity to SEQ ID NO: 49; or
   y. a VH comprising or consisting of an amino acid sequence having at least 90% sequence identity to SEQ ID NO: 25 and a VL comprising or consisting of an amino acid sequence having at least 80% sequence identity to SEQ ID NO: 50.
26. The multispecific antibody or antigen-binding fragment thereof of any previous embodiment, wherein the Fab region comprises:
   a. a VH comprising or consisting of the amino acid sequence of SEQ ID NO: 2 and a VL comprising or consisting of the amino acid sequence of SEQ ID NO: 27;
   b. a VH comprising or consisting of the amino acid sequence of SEQ ID NO: 3 and a VL comprising or consisting of the amino acid sequence of SEQ ID NO: 28;
   c. a VH comprising or consisting of the amino acid sequence of SEQ ID NO: 4 and a VL comprising or consisting of the amino acid sequence of SEQ ID NO: 29;
   d. a VH comprising or consisting of the amino acid sequence of SEQ ID NO: 5 and a VL comprising or consisting of the amino acid sequence of SEQ ID NO: 30;
   e. a VH comprising or consisting of the amino acid sequence of SEQ ID NO: 6 and a VL comprising or consisting of the amino acid sequence of SEQ ID NO: 31;
   f. a VH comprising or consisting of the amino acid sequence of SEQ ID NO: 7 and a VL comprising or consisting of the amino acid sequence of SEQ ID NO: 32;
   g. a VH comprising or consisting of the amino acid sequence of SEQ ID NO: 8 and a VL comprising or consisting of the amino acid sequence of SEQ ID NO: 33;
   h. a VH comprising or consisting of the amino acid sequence of SEQ ID NO: 9 and a VL comprising or consisting of the amino acid sequence of SEQ ID NO: 34;
   i. a VH comprising or consisting of the amino acid sequence of SEQ ID NO: 10 and a VL comprising or consisting of the amino acid sequence of SEQ ID NO: 35;
   j. a VH comprising or consisting of the amino acid sequence of SEQ ID NO: 11 and a VL comprising or consisting of the amino acid sequence of SEQ ID NO: 36;
   k. a VH comprising or consisting of the amino acid sequence of SEQ ID NO: 12 and a VL comprising or consisting of the amino acid sequence of SEQ ID NO: 37;
   l. a VH comprising or consisting of the amino acid sequence of SEQ ID NO: 13 and a VL comprising or consisting of the amino acid sequence of SEQ ID NO: 38;
   m. a VH comprising or consisting of the amino acid sequence of SEQ ID NO: 14 and a VL comprising or consisting of the amino acid sequence of SEQ ID NO: 39;
   n. a VH comprising or consisting of the amino acid sequence of SEQ ID NO: 15 and a VL comprising or consisting of the amino acid sequence of SEQ ID NO: 40;
   o. a VH comprising or consisting of the amino acid sequence of SEQ ID NO: 16 and a VL comprising or consisting of the amino acid sequence of SEQ ID NO: 41;
   p. a VH comprising or consisting of the amino acid sequence of SEQ ID NO: 17 and a VL comprising or consisting of the amino acid sequence of SEQ ID NO: 42;
   q. a VH comprising or consisting of the amino acid sequence of SEQ ID NO: 18 and a VL comprising or consisting of the amino acid sequence of SEQ ID NO: 43;
   r. a VH comprising or consisting of the amino acid sequence of SEQ ID NO: 19 and a VL comprising or consisting of the amino acid sequence of SEQ ID NO: 44;
   s. a VH comprising or consisting of the amino acid sequence of SEQ ID NO: 20 and a VL comprising or consisting of the amino acid sequence of SEQ ID NO: 45;
   t. a VH comprising or consisting of the amino acid sequence of SEQ ID NO: 21 and a VL comprising or consisting of the amino acid sequence of SEQ ID NO: 46;
   u. a VH comprising or consisting of the amino acid sequence of SEQ ID NO: 22 and a VL comprising or consisting of the amino acid sequence of SEQ ID NO: 47;
   v. a VH comprising or consisting of the amino acid sequence of SEQ ID NO: 23 and a VL comprising or consisting of the amino acid sequence of SEQ ID NO: 48;
   w. a VH comprising or consisting of the amino acid sequence of SEQ ID NO: 24 and a VL comprising or consisting of the amino acid sequence of SEQ ID NO: 49; or
   x. a VH comprising or consisting of the amino acid sequence of SEQ ID NO: 25 and a VL comprising or consisting of the amino acid sequence of SEQ ID NO: 50.
27. The multispecific antibody or antigen-binding fragment thereof of any previous embodiment, wherein the Fab region comprises an amino acid residue other than serine at position 74 according to the IMGT numbering system.
28. The multispecific antibody or antigen-binding fragment thereof of embodiment 27, wherein the residue at position 74 according to the IMGT numbering system is an amino acid selected from the group consisting of glycine, alanine, valine, methionine, leucine and isoleucine.
29. The multispecific antibody or antigen-binding fragment thereof of embodiment 27, wherein the residue at position 74 according to the IMGT numbering system is an amino acid selected from the group consisting of arginine, cysteine, glutamine, glutamic acid, glycine, histidine, isoleucine, leucine, lysine, methionine, phenylalanine, threonine, tryptophan, tyrosine and valine.
30. The multispecific antibody or antigen-binding fragment thereof of embodiment 27, wherein the residue at position 74 according to the IMGT numbering system is an amino acid selected from the group consisting of glycine, valine, methionine, leucine and isoleucine.
31. The multispecific antibody or antigen-binding fragment thereof of embodiment 27, wherein the residue at position 74 according to the IMGT numbering system is a leucine residue.
32. The multispecific antibody or antigen-binding fragment thereof of any previous embodiment, wherein the Fab region is selected from the group consisting of ADT1-4-105, ADT1-4-107, ADT1-4-110, ADT1-4-112, ADT1-4-117, ADT1-4-19, ADT1-4-21, ADT1-4-31, ADT1-4-139, ADT1-4-4, ADT1-4-143, ADT1-4-53, ADT1-4-173, ADT1-4-2, ADT1-4-8, ADT1-4-82, ADT1-4-83, ADT1-4-3, ADT1-4- 84, ADT1-4-86, ADT1-4-95, ADT1-4-1, ADT1-4-6 and ADT1-4-138.
33. The multispecific antibody or antigen-binding fragment thereof of embodiment 32, wherein the Fab region comprises from 1 to 10, from 1 to 5 or from 1 to 2 amino acid substitutions across all 6 CDR regions.
34. The multispecific antibody or antigen-binding fragment thereof of embodiment 32 or 33, wherein the Fab region comprises up to 2 or up to 1 amino acid substitution across all 6 CDR regions.
35. The multispecific antibody or antigen-binding fragment thereof of any one of embodiments 32 to 34, wherein the Fab region comprises 1 or 2 amino acid substitution across all 6 CDR regions.
36. The multispecific antibody or antigen-binding fragment thereof of any one of embodiments 32 to 35, wherein the Fab region comprises 1 to 10, 1 to 5 or 1 to 2 amino acid substitutions in one or more framework regions.
37. The multispecific antibody or antigen-binding fragment thereof of any one of embodiments 32 to 36, wherein the Fab region comprises up to 2 or up to 1 amino acid substitutions in one or more framework regions.
38. The multispecific antibody or antigen-binding fragment thereof of any one of embodiments 36 to 37, wherein the Fab region comprises 1 or 2 amino acid substitutions in one or more framework regions.
39. The multispecific antibody or antigen-binding fragment thereof of any one of embodiments 32 to 39, wherein the amino acid substitutions are conservative amino acid substitutions.
40. The multispecific antibody or antigen-binding fragment thereof of any previous embodiment, wherein the Fab region comprises:
   a. an HCDR1 sequence comprising or consisting of the sequence of GDSVSSKSXIA (SEQ ID NO: 158);
   b. an HCDR2 sequence comprising or consisting of the sequence of SEQ ID NO: 53
   c. an HCDR3 sequence comprising or consisting of the sequence of X₂WX₃X₄X₅X₆DX₇ (SEQ ID NO: 162) wherein the HCDR3 sequence is not SEQ ID NO: 54;
   d. an LCDR1 sequence comprising or consisting of the sequence of SEQ ID NO: 79;
   e. an LCDR2 sequence comprising or consisting of the sequence of SEQ ID NO: 80; and
   f. an LCDR3 sequence comprising or consisting of the sequence of QQX₈YX₉X₁₀X₁₁X₁₂X₁₃T (SEQ ID NO: 166), wherein the LCDR3 sequence is not SEQ ID NO: 81;
      wherein each of X₁ to X₁₃ is a naturally occurring amino acid.
41. The multispecific antibody or antigen-binding fragment thereof of embodiment 40, wherein:
   a. X₁ is selected from the group consisting of A and V;
   b. X₂ is selected from the group consisting of S and T;
   c. X₃ is selected from the group consisting of V, A and L;
   d. X₄ is selected from the group consisting of G, E, and D;
   e. X₅ is selected from the group consisting of Y and N;
   f. X₆ is selected from the group consisting of V, A and P;
   g. X₇ is selected from the group consisting of V, Y and R;
   h. X₈ is selected from the group consisting of K, R and G;
   i. X₉ is selected from the group consisting of S and K;
   j. X₁₀ is selected from the group consisting of T, Q, A, E and D;
   k. X₁₁ is selected from the group consisting of P, H and D;
   l. X₁₂ is selected from the group consisting of Q, R, K, W, P, E and I; and
   m. X₁₃ is selected from the group consisting of I, V and L.
42. The multispecific antibody or antigen-binding fragment thereof of embodiment 40 or embodiment 41, wherein the Fab region further comprises:
   n. an HFR1 sequence comprising or consisting of the sequence of SEQ ID NO: 170 or 171;
   o. an HFR2 sequence comprising or consisting of the sequence of SEQ ID NO: 172;
   p. an HFR3 sequence comprising or consisting of the sequence of SEQ ID NO: 173;
   q. an HFR4 sequence comprising or consisting of the sequence of SEQ ID NO: 174;
   r. an LFR1 sequence comprising or consisting of the sequence of SEQ ID NO: 175;
   s. an LFR2 sequence comprising or consisting of the sequence of SEQ ID NO: 176;
   t. an LFR3 sequence comprising or consisting of the sequence of SEQ ID NO: 177 or 178; and
   u. an LFR4 sequence comprising or consisting of the sequence of SEQ ID NO: 179, 180, 181 or 182.
43. The multispecific antibody or antigen-binding fragment thereof of any previous embodiment, wherein the Fab region thereof:
   a. binds to human TRDV1 (SEQ ID NOs 272 or 306) with a binding affinity (K_{D}, for example as measured by surface plasmon resonance) of less than about 100 nM (preferably less than about 50 nM);
   b. optionally binds to cyno TRDV1 (SEQ ID NO 308) with a binding affinity (K_{D}, for example as measured by surface plasmon resonance) of less than about 100 nM (preferably less than about 50 nM); and/or
   c. has an IC50 for TCR downregulation of less than about 50 nM (preferably less than about 10 nM.
44. The multispecific antibody or antigen-binding fragment thereof of any previous embodiment, wherein the Fab region thereof:
   a. binds to human TRDV1 with a KD of less than about 100 nM (preferably less than about 50 nM);
   b. binds to cyno TRDV1 with a KD of less than about 100 nM (preferably less than about 50 nM); and/or
   c. has an IC50 for TCR downregulation of less than about 50 nM (preferably less than about 10 nM) and an IC90 for TCR downregulation of less than about 100 nM (less than about 50 nM).
45. The multispecific antibody or antigen-binding fragment thereof of any previous embodiment, wherein:
   a. the Fab region thereof binds to human TRDV1 (SEQ ID NOs 272 or 306) with a binding affinity (K_{D}, for example as measured by surface plasmon resonance) of less than about 100 nM (preferably less than about 50 nM);
   b. optionally the Fab region thereof binds to cyno TRDV1 (SEQ ID NO 308) with a binding affinity (K_{D}, for example as measured by surface plasmon resonance) of less than about 100 nM (preferably less than about 50 nM);
   and/or
   c. the Fc region thereof binds to human PD-L1 with a binding affinity (K_{D}, for example as measured by surface plasmon resonance) of less than about 150 nM (preferably less than about 50 nM)
46. The multispecific antibody or antigen-binding fragment thereof of any previous embodiment, wherein the multispecific antibodies specifically bind to human TRDV1 with a K_{D} of less than about 5 nM and specifically bind to human PD-L1 with a K_{D} of less than about 150 nM, less than about 20 nM, less than about 10 nM or less than about 5 nM.
47. The multispecific antibody or antigen-binding fragment thereof of any previous embodiment, wherein the multispecific antibodies specifically bind to human TRDV1 with a K_{D} of less than about 5 nM and specifically bind to human PD-L1 with a K_{D} of less than about 5 nM.
48. The multispecific antibody or antigen-binding fragment thereof of embodiment 40, wherein:
   a. X₁ is selected from the group consisting of A and V;
   b. X₂ is selected from the group consisting of S and T;
   c. X₃ is selected from the group consisting of V, A and L;
   d. X₄ is selected from the group consisting of G, E, and D;
   e. X₅ is selected from the group consisting of Y and N;
   f. X₆ is selected from the group consisting of V, A and P;
   g. X₇ is selected from the group consisting of V, Y and R;
   h. X₈ is selected from the group consisting of K, R and G;
   i. X₉ is selected from the group consisting of S and K;
   j. X₁₀ is selected from the group consisting of T, Q, A, E and D;
   k. X₁₁ is selected from the group consisting of P and H;
   l. X₁₂ is selected from the group consisting of Q, R, K, W, P, E and I; and
   m. X₁₃ is selected from the group consisting of I, V and L.
49. The multispecific antibody or antigen-binding fragment thereof of embodiment 48, wherein the Fab region further comprises:
   a. an HFR1 sequence comprising or consisting of the sequence of SEQ ID NO: 170 or 171;
   b. an HFR2 sequence comprising or consisting of the sequence of SEQ ID NO: 172;
   c. an HFR3 sequence comprising or consisting of the sequence of SEQ ID NO: 173;
   d. an HFR4 sequence comprising or consisting of the sequence of SEQ ID NO: 174;
   e. an LFR1 sequence comprising or consisting of the sequence of SEQ ID NO: 175;
   f. an LFR2 sequence comprising or consisting of the sequence of SEQ ID NO: 176;
   g. an LFR3 sequence comprising or consisting of the sequence of SEQ ID NO: 177; and
   h. an LFR4 sequence comprising or consisting of the sequence of SEQ ID NO: 179, 180, 181 or 182.
50. The multispecific antibody or antigen-binding fragment thereof of any previous embodiment, wherein the Fab region thereof:
   a. binds to human TRDV1 with a K_{D} of less than about 10 nM (preferably less than about 5 nM);
   b. binds to cyno TRDV1 with a K_{D} of less than about 100 nM (preferably less than about 50 nM);
   c. has an IC50 for TCR downregulation of less than about 1 nM (preferably less than about 0.5 nM.
51. The multispecific antibody or antigen-binding fragment thereof of embodiment 40, wherein:
   a. X₁ is selected from the group consisting of A and V;
   b. X₂ is selected from the group consisting of S and T;
   c. X₃ is selected from the group consisting of V, A and L;
   d. X₄ is selected from the group consisting of G and D;
   e. X₅ is selected from the group consisting of Y and N;
   f. X₆ is selected from the group consisting of V, A and P;
   g. X₇ is selected from the group consisting of V, Y and R;
   h. X₈ is selected from the group consisting of K and R;
   i. X₉ is selected from the group consisting of S and K;
   j. X₁₀ is selected from the group consisting of T, Q, A and E;
   k. X₁₁ is selected from the group consisting of P and H;
   l. X₁₂ is selected from the group consisting of Q, K, W, P and I; and
   m. X₁₃ is selected from the group consisting of V and L.
52. The multispecific antibody or antigen-binding fragment thereof of embodiment 51, wherein the Fab region further comprises:
   a. an HFR1 sequence comprising or consisting of the sequence of SEQ ID NO: 170 or 171;
   b. an HFR2 sequence comprising or consisting of the sequence of SEQ ID NO: 172;
   c. an HFR3 sequence comprising or consisting of the sequence of SEQ ID NO: 173;
   d. an HFR4 sequence comprising or consisting of the sequence of SEQ ID NO: 174;
   e. an LFR1 sequence comprising or consisting of the sequence of SEQ ID NO: 175;
   f. an LFR2 sequence comprising or consisting of the sequence of SEQ ID NO: 176;
   g. an LFR3 sequence comprising or consisting of the sequence of SEQ ID NO: 177; and
   h. an LFR4 sequence comprising or consisting of the sequence of SEQ ID NO: 179 or 181.
53. The multispecific antibody or antigen-binding fragment thereof of any previous embodiment, wherein the Fab region thereof:
   a. binds to human TRDV1 with a K_{D} of less than about 10 nM (preferably less than about 1 nM);
   b. binds to cyno TRDV1 with a K_{D} of less than about 50 nM;
   c. has an IC50 for TCR downregulation of less than about 1 nM (preferably less than about 0.5 nM.
54. The multispecific antibody or antigen-binding fragment thereof of any previous embodiment, wherein the binding site in the Fab region binds to an epitope of the variable delta 1 (Vδ1) chain comprising one or more amino acid residues within amino acid regions 37-53 and/or 59-77 of SEQ ID NO: 272.
55. The multispecific antibody or antigen-binding fragment thereof of any previous embodiment, wherein the binding site in the Fab region binds to an epitope of the variable delta 1 (Vδ1) chain comprising amino acid residues 37, 42, 50, 53, 59, 64, 68, 69, 72, 73 and 77 of SEQ ID NO: 272.
56. The multispecific antibody or antigen-binding fragment thereof of any preceding embodiment, wherein the binding site in the Fab region binds to an epitope of the variable delta 1 (Vδ1) chain consisting of amino acid residues 37, 42, 50, 53, 59, 64, 68, 69, 72, 73 and 77 of SEQ ID NO: 272.
57. The multispecific antibody or antigen-binding fragment thereof of one of embodiments 1 to 10, wherein the Fab region comprises:
   a. a VHCDR3 comprising or consisting of an amino acid sequence having at least 90% sequence identity to any one of SEQ ID NOs: 133 to 143; and/or
   b. a VLCDR3 comprising or consisting of an amino acid sequence having at least 90% sequence identity to any one of SEQ ID NOs: 147 to 157.
58. The multispecific antibody or antigen-binding fragment thereof of embodiment 57, the Fab region comprising:
   c. a VHCDR3 comprising or consisting of the amino acid sequence of any one of SEQ ID NOs: 133 to 143; and/or
   d. a VLCDR3 comprising or consisting of the amino acid sequence of any one of SEQ ID NOs: 147 to 157.
59. The multispecific antibody or antigen-binding fragment thereof of any one of embodiments 57 to 58, the Fab region further comprising:
   a. a VHCDR1 comprising or consisting of an amino acid sequence having at least 90% sequence identity to SEQ ID NO: 130;
   b. a VHCDR2 comprising or consisting of an amino acid sequence having at least 90% sequence identity to SEQ ID NO: 131;
   c. a VLCDR1 comprising or consisting of an amino acid sequence having at least 90% sequence identity to SEQ ID NO: 144; and/or
   d. a VLCDR2 comprising or consisting of an amino acid sequence having at least 90% sequence identity to SEQ ID NO: 145.
60. The multispecific antibody or antigen-binding fragment thereof of any previous embodiment, the Fab region further comprising:
   a. a VHCDR1 comprising or consisting of the amino acid sequence of SEQ ID NO: 130;
   b. a VHCDR2 comprising or consisting of the amino acid sequence of SEQ ID NO: 131;
   c. a VLCDR1 comprising or consisting of the amino acid sequence of SEQ ID NO: 144; and/or
   d. a VLCDR2 comprising or consisting of the amino acid sequence of SEQ ID NO: 145.
61. The multispecific antibody or antigen-binding fragment thereof of any one of embodiments 57 to 60, the Fab region comprising:
   a. a VHCDR1, a VHCDR2 and a VHCDR3 comprising or consisting of the amino acid sequences of SEQ ID NO: 130, 131 and 133, respectively, and a VLCDR1, a VLCDR2 and a VLCDR3 comprising or consisting of the amino acid sequences of SEQ ID NOs: 144, 145 and 147, respectively;
   b. a VHCDR1, a VHCDR2 and a VHCDR3 comprising or consisting of the amino acid sequences of SEQ ID NO: 130, 131 and 134, respectively, and a VLCDR1, a VLCDR2 and a VLCDR3 comprising or consisting of the amino acid sequences of SEQ ID NOs: 144, 145 and 148, respectively;
   c. a VHCDR1, a VHCDR2 and a VHCDR3 comprising or consisting of the amino acid sequences of SEQ ID NO: 130, 131 and 135, respectively, and a VLCDR1, a VLCDR2 and a VLCDR3 comprising or consisting of the amino acid sequences of SEQ ID NOs: 144, 145 and 149, respectively;
   d. a VHCDR1, a VHCDR2 and a VHCDR3 comprising or consisting of the amino acid sequences of SEQ ID NO: 130, 131 and 136, respectively, and a VLCDR1, a VLCDR2 and a VLCDR3 comprising or consisting of the amino acid sequences of SEQ ID NOs: 144, 145 and 150, respectively;
   e. a VHCDR1, a VHCDR2 and a VHCDR3 comprising or consisting of the amino acid sequences of SEQ ID NO: 130, 131 and 137, respectively, and a VLCDR1, a VLCDR2 and a VLCDR3 comprising or consisting of the amino acid sequences of SEQ ID NOs: 144, 145 and 151, respectively;
   f. a VHCDR1, a VHCDR2 and a VHCDR3 comprising or consisting of the amino acid sequences of SEQ ID NO: 130, 131 and 138, respectively, and a VLCDR1, a VLCDR2 and a VLCDR3 comprising or consisting of the amino acid sequences of SEQ ID NOs: 144, 145 and 152, respectively;
   g. a VHCDR1, a VHCDR2 and a VHCDR3 comprising or consisting of the amino acid sequences of SEQ ID NO: 130, 131 and 139, respectively, and a VLCDR1, a VLCDR2 and a VLCDR3 comprising or consisting of the amino acid sequences of SEQ ID NOs: 144, 145 and 153, respectively;
   h. a VHCDR1, a VHCDR2 and a VHCDR3 comprising or consisting of the amino acid sequences of SEQ ID NO: 130, 131 and 140, respectively, and a VLCDR1, a VLCDR2 and a VLCDR3 comprising or consisting of the amino acid sequences of SEQ ID NOs: 144, 145 and 154, respectively;
   i. a VHCDR1, a VHCDR2 and a VHCDR3 comprising or consisting of the amino acid sequences of SEQ ID NO: 130, 131 and 141, respectively, and a VLCDR1, a VLCDR2 and a VLCDR3 comprising or consisting of the amino acid sequences of SEQ ID NOs: 144, 145 and 155, respectively;
   j. a VHCDR1, a VHCDR2 and a VHCDR3 comprising or consisting of the amino acid sequences of SEQ ID NO: 130, 131 and 142, respectively, and a VLCDR1, a VLCDR2 and a VLCDR3 comprising or consisting of the amino acid sequences of SEQ ID NOs: 144, 145 and 156, respectively; or
   k. a VHCDR1, a VHCDR2 and a VHCDR3 comprising or consisting of the amino acid sequences of SEQ ID NO: 130, 131 and 143, respectively, and a VLCDR1, a VLCDR2 and a VLCDR3 comprising or consisting of the amino acid sequences of SEQ ID NOs: 144, 145 and 157, respectively.
62. The multispecific antibody or antigen-binding fragment thereof of any one of embodiments 57 to 61, the Fab region comprising
   a. a VH comprising or consisting of an amino acid sequence having at least 90% sequence identity to any one of SEQ ID NOs: 107 to 117; and/or
   b. a VL comprising or consisting of an amino acid sequence having at least 90% sequence identity to any one of SEQ ID NOs: 119 to 129.
63. The multispecific antibody or antigen-binding fragment thereof of any one of embodiments 57 to 62, the Fab region comprising:
   a. a VH comprising or consisting of the amino acid sequence of any one of SEQ ID NOs: 107 to 117; and/or
   b. a VL comprising or consisting of the amino acid sequence of any one of SEQ ID NOs: 119 to 129.
64. The multispecific antibody or antigen-binding fragment thereof of any of embodiments 57 to 63, the Fab region comprising:
   a. a VH comprising or consisting of an amino acid sequence having at least 90% sequence identity to SEQ ID NO: 107 and a VL comprising or consisting of an amino acid sequence having at least 90% sequence identity to SEQ ID NO: 119;
   b. a VH comprising or consisting of an amino acid sequence having at least 90% sequence identity to SEQ ID NO: 108 and a VL comprising or consisting of an amino acid sequence having at least 90% sequence identity to SEQ ID NO: 120;
   c. a VH comprising or consisting of an amino acid sequence having at least 90% sequence identity to SEQ ID NO: 109 and a VL comprising or consisting of an amino acid sequence having at least 90% sequence identity to SEQ ID NO: 121;
   d. a VH comprising or consisting of an amino acid sequence having at least 90% sequence identity to SEQ ID NO: 110 and a VL comprising or consisting of an amino acid sequence having at least 90% sequence identity to SEQ ID NO: 122;
   e. a VH comprising or consisting of an amino acid sequence having at least 90% sequence identity to SEQ ID NO: 111 and a VL comprising or consisting of an amino acid sequence having at least 90% sequence identity to SEQ ID NO: 123;
   f. a VH comprising or consisting of an amino acid sequence having at least 90% sequence identity to SEQ ID NO: 112 and a VL comprising or consisting of an amino acid sequence having at least 90% sequence identity to SEQ ID NO: 124;
   g. a VH comprising or consisting of an amino acid sequence having at least 90% sequence identity to SEQ ID NO: 113 and a VL comprising or consisting of an amino acid sequence having at least 90% sequence identity to SEQ ID NO: 125;
   h. a VH comprising or consisting of an amino acid sequence having at least 90% sequence identity to SEQ ID NO: 114 and a VL comprising or consisting of an amino acid sequence having at least 90% sequence identity to SEQ ID NO: 126;
   i. a VH comprising or consisting of an amino acid sequence having at least 90% sequence identity to SEQ ID NO: 115 and a VL comprising or consisting of an amino acid sequence having at least 90% sequence identity to SEQ ID NO: 127;
   j. a VH comprising or consisting of an amino acid sequence having at least 90% sequence identity to SEQ ID NO: 116 and a VL comprising or consisting of an amino acid sequence having at least 90% sequence identity to SEQ ID NO: 128; or
   k. a VH comprising or consisting of an amino acid sequence having at least 90% sequence identity to SEQ ID NO: 117 and a VL comprising or consisting of an amino acid sequence having at least 90% sequence identity to SEQ ID NO: 129.
65. The multispecific antibody or antigen-binding fragment thereof of any one of embodiments 57 to 64, the Fab region comprising
   a. a VH comprising or consisting of the amino acid sequence of SEQ ID NO: 107 and a VL comprising or consisting of the amino acid sequence of SEQ ID NO: 119;
   b. a VH comprising or consisting of the amino acid sequence of SEQ ID NO: 108 and a VL comprising or consisting of the amino acid sequence of SEQ ID NO: 120;
   c. a VH comprising or consisting of the amino acid sequence of SEQ ID NO: 109 and a VL comprising or consisting of the amino acid sequence of SEQ ID NO: 121;
   d. a VH comprising or consisting of the amino acid sequence of SEQ ID NO: 110 and a VL comprising or consisting of the amino acid sequence of SEQ ID NO: 122;
   e. a VH comprising or consisting of the amino acid sequence of SEQ ID NO: 111 and a VL comprising or consisting of the amino acid sequence of SEQ ID NO: 123;
   f. a VH comprising or consisting of the amino acid sequence of SEQ ID NO: 112 and a VL comprising or consisting of the amino acid sequence of SEQ ID NO: 124;
   g. a VH comprising or consisting of the amino acid sequence of SEQ ID NO: 113 and a VL comprising or consisting of the amino acid sequence of SEQ ID NO: 125;
   h. a VH comprising or consisting of the amino acid sequence of SEQ ID NO: 114 and a VL comprising or consisting of the amino acid sequence of SEQ ID NO: 126;
   i. a VH comprising or consisting of the amino acid sequence of SEQ ID NO: 115 and a VL comprising or consisting of the amino acid sequence of SEQ ID NO: 127;
   j. a VH comprising or consisting of the amino acid sequence of SEQ ID NO: 116 and a VL comprising or consisting of the amino acid sequence of SEQ ID NO: 128; or
   k. a VH comprising or consisting of the amino acid sequence of SEQ ID NO: 117 and a VL comprising or consisting of the amino acid sequence of SEQ ID NO: 129.
66. The multispecific antibody or antigen-binding fragment thereof of any one of embodiments 57 to 65, wherein the Fab region comprises a kappa light chain variable sequence having an amino acid residue other than serine at position 74 according to the IMGT numbering system.
67. The multispecific antibody or antigen-binding fragment thereof of embodiment 66, wherein the residue at position 74 according to the IMGT numbering system of the light chain variable sequence is an amino acid selected from the group consisting of glycine, alanine, valine, methionine, leucine and isoleucine.
68. The multispecific antibody or antigen-binding fragment thereof of embodiment 66, wherein the residue at position 74 according to the IMGT numbering system of the light chain variable sequence is an amino acid selected from the group consisting of arginine, cysteine, glutamine, glutamic acid, glycine, histidine, isoleucine, leucine, lysine, methionine, phenylalanine, threonine, tryptophan, tyrosine and valine.
69. The multispecific antibody or antigen-binding fragment thereof of embodiment 66, wherein the residue at position 74 according to the IMGT numbering system of the light chain variable sequence is an amino acid selected from the group consisting of glycine, valine, methionine, leucine and isoleucine.
70. The multispecific antibody or antigen-binding fragment thereof of embodiment 66, wherein the residue at position 74 according to the IMGT numbering system of the light chain variable sequence is a leucine residue.
71. The multispecific antibody or antigen-binding fragment thereof of any one of embodiments 57 to 70, wherein the Fab region is selected from the group consisting of ADT1-7-10, ADT1-7-15, ADT1-7-17, ADT1-7-18, ADT1-7-19, ADT1-7-20, ADT1-7-22 ADT1-7-23, ADT1-7-42, ADT1-7-3 and ADT1-7-61.
72. The multispecific antibody or antigen-binding fragment thereof of embodiment 71, wherein the Fab region comprises from 1 to 10, from 1 to 5 or from 1 to 2 amino acid substitutions across all 6 CDR regions.
73. The multispecific antibody or antigen-binding fragment thereof of embodiment 71 or 72, wherein the Fab region comprises up to 2 or up to 1 amino acid substitution across all 6 CDR regions.
74. The multispecific antibody or antigen-binding fragment thereof of any one of embodiments 71 to 73, wherein the Fab region comprises 1 or 2 amino acid substitution across all 6 CDR regions.
75. The multispecific antibody or antigen-binding fragment thereof of any one of embodiments 71 to 74, wherein the Fab region comprises 1 to 10, 1 to 5 or 1 to 2 amino acid substitutions in one or more framework regions.
76. The multispecific antibody or antigen-binding fragment thereof of any one of embodiments 71 to 75, wherein the Fab region comprises up to 2 or up to 1 amino acid substitutions in one or more framework regions.
77. The multispecific antibody or antigen-binding fragment thereof of any one of embodiments 71 to 76, wherein the Fab region comprises 1 or 2 amino acid substitutions in one or more framework regions.
78. The multispecific antibody or antigen-binding fragment thereof of any one of embodiments 71 to 6077 wherein the amino acid substitutions are conservative amino acid substitutions.
79. The multispecific antibody or antigen-binding fragment thereof of one of embodiments 1 to 14, wherein the Fab region comprises:
   a. an HCDR1 sequence comprising or consisting of the sequence of SEQ ID NO: 130
   b. an HCDR2 sequence comprising or consisting of the sequence of SEQ ID NO: 131
   c. an HCDR3 sequence comprising or consisting of the sequence of X₁X₂YX₃X₄AFDI, wherein the HCDR3 sequence is not SEQ ID NO: 132;
   d. an LCDR1 sequence comprising or consisting of the sequence of SEQ ID NO: 144;
   e. an LCDR2 sequence comprising or consisting of the sequence of SEQ ID NO: 145; and
   f. an LCDR3 sequence comprising or consisting of the sequence of QQX₅X₆X₇X₈LX₉T, wherein the LCDR3 sequence is no SEQ ID NO: 146;
      wherein each of X₁ to X₉ is a naturally occurring amino acid.
80. The multispecific antibody or antigen-binding fragment thereof of embodiment 79, wherein:
   a. X₁ is selected from the group consisting of D, I and V;
   b. X₂ is selected from the group consisting of D and S;
   c. X₃ is selected from the group consisting of N, E, D, Q and A;
   d. X₄ is selected from the group consisting of D and E;
   e. X₅ is selected from the group consisting of T and S;
   f. X₆ is selected from the group consisting of A, G, Y and S;
   g. X₇ is selected from the group consisting of S and D;
   h. X₈ is selected from the group consisting of T, E and G; and
   i. X₉ is selected from the group consisting of L and D.
81. The multispecific antibody or antigen-binding fragment thereof of embodiment 79 or embodiment 80, wherein the Fab region further comprises:
   a. an HFR1 sequence comprising or consisting of the sequence of SEQ ID NO: 189;
   b. an HFR2 sequence comprising or consisting of the sequence of SEQ ID NO: 190;
   c. an HFR3 sequence comprising or consisting of the sequence of SEQ ID NO: 191;
   d. an HFR4 sequence comprising or consisting of the sequence of SEQ ID NO: 192;
   e. an LFR1 sequence comprising or consisting of the sequence of SEQ ID NO: 193 and 194;
   f. an LFR2 sequence comprising or consisting of the sequence of SEQ ID NO: 195;
   g. an LFR3 sequence comprising or consisting of the sequence of SEQ ID NO: 196; and
   h. an LFR4 sequence comprising or consisting of the sequence of SEQ ID NO: 197.
82. The multispecific antibody or antigen-binding fragment thereof of any one of embodiments 57 to 81, wherein the antibody or antigen-binding fragment thereof:
   a. binds to human TRDV1 with a K_{D} of less than about 10 nM (preferably less than about 5 nM);
   b. has an IC50 for TCR downregulation of less than about 50 nM (preferably less than about 10 nM).
83. The multispecific antibody or antigen-binding fragment thereof of embodiment 79, wherein:
   a. X₁ is selected from the group consisting of D and V;
   b. X₂ is selected from the group consisting of D and S;
   c. X₃ is selected from the group consisting of D, Q and A;
   d. X₄ is selected from the group consisting of D and E;
   e. X₅ is S;
   f. X₆ is selected from the group consisting of A and Y;
   g. X₇ is S;
   h. X₈ is selected from the group consisting of E and G; and
   i. X₉ is selected from the group consisting of L and D.
84. The multispecific antibody or antigen-binding fragment thereof of embodiment 83, wherein the Fab region further comprises:
   a. an HFR1 sequence comprising or consisting of the sequence of SEQ ID NO: 189;
   b. an HFR2 sequence comprising or consisting of the sequence of SEQ ID NO: 190;
   c. an HFR3 sequence comprising or consisting of the sequence of SEQ ID NO: 191;
   d. an HFR4 sequence comprising or consisting of the sequence of SEQ ID NO: 192;
   e. an LFR1 sequence comprising or consisting of the sequence of SEQ ID NO: 193;
   f. an LFR2 sequence comprising or consisting of the sequence of SEQ ID NO: 195;
   g. an LFR3 sequence comprising or consisting of the sequence of SEQ ID NO: 196; and
   h. an LFR4 sequence comprising or consisting of the sequence of SEQ ID NO: 197.
85. The multispecific antibody or antigen-binding fragment thereof of any one of embodiments 57 to 84, wherein the Fab region thereof:
   a. binds to human TRDV1 with a K_{D} of less than about 10 nM (preferably less than about 5 nM); and/or
   b. has an IC50 for TCR downregulation of less than about 5 nM (preferably less than about 1 nM).
86. The multispecific antibody or antigen-binding fragment thereof of any one of embodiments 57 to 85, wherein the binding site in the Fab region binds to an epitope of the variable delta 1 (Vδ1) chain comprising one or more amino acid residues within amino acid regions 5-20 and/or 62-77 of SEQ ID NO: 271.
87. The multispecific antibody or antigen-binding fragment thereof of any one of embodiments 57 to 86, wherein the binding site in the Fab region binds to an epitope of the variable delta 1 (Vδ1) chain comprising amino acid residues 5, 9, 16, 20, 62, 64, 72 and 77 of SEQ ID NO: 272.
88. The multispecific antibody of any one of embodiments 57 to 87, wherein the binding site in the Fab region binds to an epitope of the variable delta 1 (Vδ1) chain consisting of amino acid residues 5, 9, 16, 20, 62, 64, 72 and 77 of SEQ ID NO: 272.
89. The multispecific antibody or antigen-binding fragment thereof of any one of embodiments 1 to 14, wherein the Fab region is derived from an affinity matured variant of a parent anti-Vδ1 antibody, wherein the parent anti-Vδ1 antibody comprises a VH comprising the amino acid sequence of SEQ ID NO: 1 and a VL sequence comprising the amino acid sequence of SEQ ID NO: 26.
90. The multispecific antibody or antigen-binding fragment thereof of embodiment 89, wherein the Fab region is as defined in any previous embodiment.
91. The multispecific antibody or antigen-binding fragment thereof of any previous embodiment, wherein the Fab region thereof:
   a. binds to human TRDV1 with a KD of less than about 100 nM (preferably less than about 50 nM);
   b. binds to cyno TRDV1 with a KD of less than about 100 nM (preferably less than about 50 nM); and/or
   c. has an IC50 for TCR downregulation of less than about 50 nM (preferably less than about 10 nM).
92. The multispecific antibody or antigen-binding fragment thereof of any previous embodiment, wherein Fab region:
   a. binds to human TRDV1 with a K_{D} of less than about 10 nM (preferably less than about 5 nM);
   b. binds to cyno TRDV1 with a K_{D} of less than about 100 nM (preferably less than about 50 nM); and/or
   c. has an IC50 for TCR downregulation of less than about 1 nM (preferably less than about 0.5 nM).
93. The multispecific antibody or antigen-binding fragment thereof of any previous embodiment, wherein Fab region:
   a. binds to human TRDV1 with a K_{D} of less than about 10 nM (preferably less than about 1 nM);
   b. binds to cyno TRDV1 with a K_{D} of less than about 50 nM; and/or
   c. has an IC50 for TCR downregulation of less than about 1 nM (preferably less than about 0.5 nM).
94. The multispecific antibody or antigen-binding fragment thereof of any one of embodiments 1 to 14, wherein the Fab region is derived from an affinity matured variant of a parent anti-Vδ1 antibody, wherein the parent anti-Vδ1 antibody or antigen-binding fragment thereof comprises a VH comprising the amino acid sequence of SEQ ID NO: 106 and a VL sequence comprising the amino acid sequence of SEQ ID NO: 118.
95. The multispecific antibody or antigen-binding fragment thereof of embodiment 94, wherein the Fab region is as defined in any one previous embodiment.
96. The multispecific antibody or antigen-binding fragment thereof of any previous embodiment, wherein the antibody or antigen-binding fragment thereof:
   a. binds to human TRDV1 with a K_{D} of less than about 10 nM (preferably less than about 5 nM); and/or
   b. has an IC50 for TCR downregulation of less than about 50 nM (preferably less than about 10 nM).
97. The multispecific antibody or antigen-binding fragment thereof of any previous embodiment, wherein the Fab region:
   a. binds to human TRDV1 with a K_{D} of less than about 10 nM (preferably less than about 5 nM); and/or
   b. has an IC50 for TCR downregulation of less than about 5 nM (preferably less than about 1 nM).
98. The multispecific antibody or antigen-binding fragment thereof of any one of embodiments 1 to 14 wherein the Fab region is an affinity matured variant of a parent anti-Vδ1 antibody, wherein the parent anti-Vδ1 antibody or antigen-binding fragment thereof comprises:
   a. a VH comprising or consisting of the amino acid sequence of SEQ ID NO: 273 and a VL comprising or consisting of the amino acid sequence of SEQ ID NO: 282;
   b. a VH comprising or consisting of the amino acid sequence of SEQ ID NO: 274 and a VL comprising or consisting of the amino acid sequence of SEQ ID NO: 283;
   c. a VH comprising or consisting of the amino acid sequence of SEQ ID NO: 275 and a VL comprising or consisting of the amino acid sequence of SEQ ID NO: 284;
   d. a VH comprising or consisting of the amino acid sequence of SEQ ID NO: 276 and a VL comprising or consisting of the amino acid sequence of SEQ ID NO: 285;
   e. a VH comprising or consisting of the amino acid sequence of SEQ ID NO: 277 and a VL comprising or consisting of the amino acid sequence of SEQ ID NO: 286;
   f. a VH comprising or consisting of the amino acid sequence of SEQ ID NO: 278 and a VL comprising or consisting of the amino acid sequence of SEQ ID NO: 287;
   g. a VH comprising or consisting of the amino acid sequence of SEQ ID NO: 279 and a VL comprising or consisting of the amino acid sequence of SEQ ID NO: 288;
   h. a VH comprising or consisting of the amino acid sequence of SEQ ID NO: 280 and a VL comprising or consisting of the amino acid sequence of SEQ ID NO: 289;
   i. a VH comprising or consisting of the amino acid sequence of SEQ ID NO: 281 and a VL comprising or consisting of the amino acid sequence of SEQ ID NO: 290; or
   j. a VH comprising or consisting of the amino acid sequence of SEQ ID NO: 312 and a VL comprising or consisting of the amino acid sequence of SEQ ID NO: 313.
99. The multispecific antibody or antigen-binding fragment thereof of any previous embodiment, wherein the affinity matured variant has at least 20%, at least 30%, at least 40%, at least 50%, at least 100%, at least 500% or more preferably at least about 1000% greater affinity than the parental antibody for binding to a variable delta 1 (Vδ1) chain of a γδ T cell receptor (TCR), for example as measured by the Kd.
100. The multispecific antibody or antigen-binding fragment thereof of any previous embodiment, wherein the affinity matured variant comprises VH and VL sequences that are at least 80%, at least 90%, at least 95% or at least 96% identical to the corresponding parental VH and VL sequences.
101. The multispecific antibody or antigen-binding fragment thereof of any previous embodiment, wherein the affinity variant comprises up to 20, for example up to 15, for example up to 10 amino acid substitutions compared to the parental antibody sequences.
102. The multispecific antibody of any one of embodiments 1 to 14, the Fab region comprising a kappa light chain variable sequence, in which the residue at position 74 according to the IMGT numbering system is not a serine reside.
103. The multispecific antibody or antigen-binding fragment thereof of embodiment 102, wherein the residue at position 74 according to the IMGT numbering system is an amino acid selected from the group consisting of glycine, alanine, valine, methionine, leucine and isoleucine.
104. The multispecific antibody or antigen-binding fragment thereof of embodiment 102, wherein the residue at position 74 according to the IMGT numbering system is an amino acid selected from the group consisting of arginine, cysteine, glutamine, glutamic acid, glycine, histidine, isoleucine, leucine, lysine, methionine, phenylalanine, threonine, tryptophan, tyrosine and valine.
105. The multispecific antibody or antigen-binding fragment thereof of embodiment 102, wherein the residue at position 74 according to the IMGT numbering system is an amino acid selected from the group consisting of glycine, valine, methionine, leucine and isoleucine.
106. The multispecific antibody or antigen-binding fragment thereof of embodiment 102, wherein the residue at position 74 according to the IMGT numbering system is a leucine residue.
107. The multispecific antibody or antigen-binding fragment thereof of any preceding embodiment, wherein the antibody is an IgG antibody.
108. The multispecific antibody or antigen-binding fragment thereof of any preceding embodiment, wherein the antibody is an IgG1 antibody.
109. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 564.
110. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 568.
111. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 570.
112. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 573.
113. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 574.
114. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 576.
115. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 577.
116. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 579.
117. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 581.
118. The multispecific antibody or antigen-binding fragment thereof of any previous embodiment, wherein the Fab region comprises a VH comprising the amino acid sequence of SEQ ID NO: 15 and a VL sequence comprising the amino acid sequence of SEQ ID NO: 40; and the Fc region comprises a CH3 domain comprising the amino acid sequence of SEQ ID NO: 583.
119. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 564.
120. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 568.
121. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 570.
122. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 573.
123. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 574.
124. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 576.
125. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 577.
126. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 579.
127. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 581.
128. The multispecific antibody or antigen-binding fragment thereof of any previous embodiment, wherein the Fab region comprises a VH comprising the amino acid sequence of SEQ ID NO: 23 and a VL sequence comprising the amino acid sequence of SEQ ID NO: 48; and the Fc region comprises a CH3 domain comprising the amino acid sequence of SEQ ID NO: 583.
129. A multispecific antibody or antigen-binding fragment thereof of any previous embodiment, wherein the Fab region specifically binds to a variable delta 1 (Vδ1) chain of a γδ T cell receptor (TCR) and competes with binding to the variable delta 1 (Vδ1) chain of a γδ T cell receptor (TCR) with an antibody or antigen-binding fragment thereof of any preceding embodiment.
130. The multispecific antibody or antigen-binding fragment thereof of any previous embodiment, wherein the antibody is Fc-enabled.
131. A multispecific antibody or antigen-binding fragment thereof of any previous embodiment, characterised in that it:
   a. causes downregulation of TCRs on Vδ1 T-cells to which it binds;
   b. does not exhibit CDC or ADCC; and
   c. does not deplete Vδ1 T-cells.
132. The multispecific antibody or antigen-binding fragment thereof of embodiment 131 wherein the antibody causes a depletion of less than about 30%, or less than about 20%, or less than about 10% of the viable Vδ1 T+ cell population via ADCC and/or CDC.
133. The multispecific antibody or antigen-binding fragment thereof of embodiment 131, wherein the antibody does not induce secretion of IL-17A.
134. The multispecific antibody or antigen-binding fragment thereof of embodiment 131, wherein the antibody induces less than about 30%, or less than about 20%, or less than about 10% of the IL-17A secretion induced by equivalent CD3 antibodies.
135. The multispecific antibody or antigen-binding fragment thereof of any previous embodiments, wherein the Fab region binds to human TRVD1 with a KD of less than about 10 nM.
136. The multispecific antibody or antigen-binding fragment thereof of any previous embodiments, wherein the Fab region binds to human TRVD1 with a KD of less than about 5 nM.
137. The multispecific antibody or antigen-binding fragment thereof of any previous embodiments, wherein the Fab region binds to cyno TRVD1 with a KD of less than about 100 nM.
138. The multispecific antibody of embodiment 137, wherein the Fab region binds to cyno TRVD1 with a KD of less than about 50 nM.
139. A multispecific antibody or antigen-binding fragment thereof according to any previous embodiment, wherein that the Fab region specifically binds to a human variable delta 1 (Vδ1) chain of a γδ T cell receptor (TCR) with a KD of less than about 10 nM.
140. The multispecific antibody or antigen-binding fragment thereof of any previous embodiment, wherein the Fab region is as defined in any one of embodiments 1 to 139.
141. The multispecific antibody or antigen-binding fragment thereof of any preceding embodiment, wherein the Fab region binds to a cyno variable delta 1 (Vδ1) chain of a γδ T cell receptor (TCR) with a KD of less than about 100 nM or less than about 50 nM.
142. The multispecific antibody or antigen-binding fragment thereof of any preceding embodiment, wherein the Fab region has an IC50 for TCR downregulation of less than about 50 nM, preferably less than about 10 nM.
143. The multispecific antibody or antigen-binding fragment thereof of any preceding embodiment, wherein the multispecific antibody has an IC50 for A375 cell killing of less than about 10 nM, preferably less than about 5 nM.
144. The multispecific antibody or antigen-binding fragment thereof of any preceding embodiment, wherein the Fab region stimulates Vδ1 T-cell proliferation.
145. The multispecific antibody or antigen-binding fragment thereof of any preceding embodiment, wherein the Fab region binds human TVRD1 with a KD of less than about 100 nM.
146. The multispecific antibody or antigen-binding fragment thereof of any preceding embodiment, wherein the Fab region binds human TVRD1 with a KD of less than about 50 nM.
147. The multispecific antibody or antigen-binding fragment thereof of any preceding embodiment, wherein the Fab region binds human TRDV1 with K_{D} of less than about 10 nM.
148. The multispecific antibody or antigen-binding fragment thereof of any preceding embodiment, wherein the Fab region binds human TRDV1 with K_{D} of less than about 5 nM.
149. The multispecific antibody or antigen-binding fragment thereof of any preceding embodiment, wherein the Fab region binds human TRDV1 with K_{D} of less than about 1 nM.
150. The multispecific antibody or antigen-binding fragment thereof of any preceding embodiment, wherein the Fab region binds cyno TRDV1 with K_{D} of less than about 100 nM.
151. The multispecific antibody or antigen-binding fragment thereof of any preceding embodiment, wherein the Fab region binds cyno TRDV1 with K_{D} of less than about 50 nM.
152. The multispecific antibody or antigen-binding fragment thereof of any preceding embodiment, wherein the Fab region has an IC50 for TCR downregulation of less than about 50 nM, less than about 10 nM, less than about 5 nM, less than about 1 nM or less than about 0.5 nM.
153. The multispecific antibody or antigen-binding fragment thereof of any preceding embodiment, wherein the Fab region:
   a. binds to human TRDV1 (SEQ ID NOs 272 or 306) with a binding affinity (K_{D}, for example as measured by surface plasmon resonance) of less than about 100 nM (preferably less than about 50 nM);
   b. optionally binds to cyno TRDV1 (SEQ ID NO 308) with a binding affinity (K_{D}, for example as measured by surface plasmon resonance) of less than about 100 nM (preferably less than about 50 nM);
   c. has an IC50 for TCR downregulation of less than about 50 nM (preferably less than about 10 nM).
154. The multispecific antibody or antigen-binding fragment thereof of any preceding embodiment, wherein the Fab region:
   a. binds to human TRDV1 with a K_{D} of less than about 100 nM (preferably less than about 50 nM);
   b. binds to cyno TRDV1 with a K_{D} of less than about 100 nM (preferably less than about 50 nM);
   c. has an IC50 for TCR downregulation of less than about 50 nM (preferably less than about 10 nM) and an IC90 for TCR downregulation of less than about 100 nM (less than about 50 nM).
155. The multispecific antibody or antigen-binding fragment thereof of any preceding embodiment, wherein the Fab region:
   a. binds to human TRDV1 with a K_{D} of less than about 10 nM (preferably less than about 5 nM); and/or
   b. binds to cyno TRDV1 with a K_{D} of less than about 100 nM (preferably less than about 50 nM).
156. The multispecific antibody or antigen-binding fragment thereof of any preceding embodiment, wherein the Fab region:
   a. binds to human TRDV1 with a K_{D} of less than about 10 nM (preferably less than about 1 nM);
   b. binds to cyno TRDV1 with a K_{D} of less than about 50 nM;
   c. has an IC50 for TCR downregulation of less than about 1 nM (preferably less than about 0.5 nM).
157. The multispecific antibody or antigen-binding fragment thereof of any preceding embodiment, wherein the Fab region:
   a. binds to human TRDV1 with a K_{D} of less than about 10 nM (preferably less than about 5 nM);
   b. have an IC50 for TCR downregulation of less than about 50 nM (preferably less than about 10 nM).
158. The multispecific antibody or antigen-binding fragment thereof of any preceding embodiment, wherein the Fab region:
   a. binds to human TRDV1 with a K_{D} of less than about 10 nM (preferably less than about 5 nM);
   b. has an IC50 for TCR downregulation of less than about 5 nM (preferably less than about 1 nM); and
159. The multispecific antibody or antigen-binding fragment thereof of any preceding embodiment, wherein the Fab region is a human Fab region.
160. The multispecific antibody or antigen-binding fragment thereof of any preceding embodiment, wherein the antibody is a mAb².
161. The multispecific antibody or antigen-binding fragment thereof of any preceding embodiment, wherein the antibody is a monoclonal antibody.
162. The multispecific antibody or antigen-binding fragment thereof of any preceding embodiment, wherein the Fab region only binds to an epitope in the V region of a Vδ1 chain of a γδ TCR.
163. The multispecific antibody or antigen-binding fragment thereof of any preceding embodiment, wherein the Fab region only binds to an epitope within amino acid residues 1-90 of SEQ ID NO: 272, SEQ ID NO: 306 or SEQ ID NO: 308.
164. The multispecific antibody or antigen-binding fragment thereof of any preceding embodiment, wherein the Fab region does not bind to an epitope found in CDR3 of a Vδ1 chain of a γδ TCR.
165. The multispecific antibody or antigen-binding fragment thereof of any preceding embodiment, wherein the Fab region thereof does not bind to an epitope within amino acid region 91-105 (CDR3) of SEQ ID NO: 272.
166. The multispecific antibody or antigen-binding fragment thereof of any preceding embodiment, wherein the Fab region binds to an epitope within amino acid region
   (i) 3-20 of SEQ ID NO: 272; and/or
   (ii) 37-77 of SEQ ID NO: 272.
167. The multispecific antibody or antigen-binding fragment thereof or of embodiment 166, wherein the epitope bound by the Fab region comprises one or more amino acid residues within amino acid regions 37-53 and/or 59-77 of SEQ ID NO: 272.
168. The multispecific antibody or antigen-binding fragment thereof of embodiment 166, wherein the epitope bound by the Fab region comprises amino acid residues 37, 42, 50, 53, 59, 64, 68, 69, 72, 73 and 77 of SEQ ID NO: 272.
169. The multispecific antibody or antigen-binding fragment thereof of embodiment 166, wherein the epitope bound by the Fab region consists of amino acid residues 37, 42, 50, 53, 59, 64, 68, 69, 72, 73 and 77 of SEQ ID NO: 272.
170. The multispecific antibody or antigen-binding fragment thereof of embodiment 166, wherein the epitope bound by the Fab region comprises one or more amino acid residues within amino acid regions 5-20 and/or 62-77 of SEQ ID NO: 272.
171. The multispecific antibody or antigen-binding fragment thereof of embodiment 166, wherein the epitope bound by the Fab region comprises amino acid residues 5, 9, 16, 20, 62, 64, 72 and 77 of SEQ ID NO: 272.
172. The multispecific antibody or antigen-binding fragment thereof of embodiment 166, wherein the epitope bound by the Fab region consists of amino acid residues 5, 9, 16, 20, 62, 64, 72 and 77 of SEQ ID NO: 272.
173. The multispecific antibody or antigen-binding fragment thereof of embodiment 166, wherein the epitope bound by the Fab region is an activating epitope of a γδ T cell.
174. The multispecific antibody or antigen-binding fragment thereof of embodiment 173, wherein binding of the activating epitope: (i) downregulates the γδ TCR; (ii) activates degranulation of the γδ T cell; and/or (iii) promotes γδ T cell mediated killing.
175. The multispecific antibody or antigen-binding fragment thereof of any previous embodiment, wherein binding of the activating epitope upregulates expression of CD107a, CD25, CD69 and/or Ki-67.
176. The multispecific antibody or antigen-binding fragment thereof of any preceding embodiment, wherein the multispecific antibody is larger than about 70 KDa.
177. The multispecific antibody or antigen-binding fragment thereof of any preceding embodiment, wherein the antibody does not comprise an VHCDR3 having SEQ ID NO: 54 and a VLCDR3 having SEQ ID NO: 81, and/or wherein the antibody not comprise an VHCDR3 having SEQ ID NO: 132 and a VLCDR3 having SEQ ID NO: 146.
178. The multispecific antibody or antigen-binding fragment thereof of any preceding embodiment, wherein the antibody does not comprise a VH region having SEQ ID NO: 1 and a VL region having SEQ ID NO: 26, and/or wherein the antibody does not comprise a VH region having SEQ ID NO: 106 and a VL region having SEQ ID NO: 118.
179. A polynucleotide sequence encoding the multispecific antibody or antigen-binding fragment thereof as defined in any preceding embodiment.
180. A polynucleotide sequence encoding the anti-Vδ1 first binding domain comprising a sequence having at least 70% sequence identity with SEQ ID NO: 199 to 222, 224 to 247, 249 to 259 or 261 to 271.
181. An expression vector comprising the polynucleotide sequence as defined in embodiment 179 or embodiment 180.
182. A host cell comprising the polynucleotide sequence as defined embodiment 179 or embodiment 180 or the expression vector as defined in embodiment 168, optionally wherein the host cell is a recombinant host cell.
183. The host cell of embodiment 182, comprising a first polynucleotide sequence encoding a VH sequence, and a second polynucleotide sequence encoding a VL sequence.
184. The host cell of embodiment 182, comprising a first expression vector comprising a first polynucleotide sequence encoding a VH sequence, and a second expression vector comprising a second polynucleotide sequence encoding a VL sequence.
185. The host cell of embodiment 184, comprising an expression vector comprising a first polynucleotide sequence encoding a VH sequence and a second polynucleotide sequence encoding a VL sequence.
186. The host cell as defined in any one of embodiments 182 to 185, wherein the polynucleotide or expression vector encodes a membrane anchor or transmembrane domain fused to the antibody or antigen-binding fragment thereof, wherein the antibody or antigen-binding fragment thereof is presented on an extracellular surface of the cell
187. A method for producing any multispecific antibody of any one of embodiments 1 to 178, comprising culturing a host cell of any one of embodiments 182 to 186 in a cell culture medium.
188. The method of embodiment 187, further comprising isolating the multispecific antibody or antigen-binding fragment thereof from the culture medium.
189. A composition comprising the multispecific antibody or antigen-binding fragment thereof as defined in any one of embodiments 1 to 178.
190. A pharmaceutical composition comprising the multispecific antibody or antigen-binding fragment thereof as defined in any one of embodiments 1 to 178 and a pharmaceutically acceptable diluent or carrier.
191. A kit comprising the multispecific antibody or antigen-binding fragment thereof of any one of embodiments 1 to 178 or a pharmaceutical composition of embodiment 190.
192. The kit of embodiment 191, further comprising an additional therapeutically active agent.
193. The kit of embodiment 191 or embodiment 192, further comprising instructions for use.
194. A method of treating a disease or disorder in a subject, comprising administering to the subject multispecific antibody of any one of embodiments 1 to 178, or the pharmaceutical composition of embodiment 190.
195. The method of embodiment 194, wherein the disease or disorder is cancer.
196. The method of embodiment 195, wherein the cancer is a PD-L1 positive cancer.
197. The method of embodiment 196, wherein the cancer is selected from the group consisting of head and neck squamous cell carcinoma (HNSCC), non-small cell lung cancer (NSCLC), colorectal cancer (CRC), and gastric cancers.
198. The method of any one of embodiments 194 to 197, further comprising administering simultaneously or sequentially in any order, a second agent to the subject.
199. A method of modulating an immune response in a subject, comprising administering to the subject the multispecific antibody or antigen-binding fragment thereof of any one of embodiments 1 to 178, or the pharmaceutical composition of embodiment 190.
200. The method of embodiment 199, wherein the subject has cancer.
201. The method of embodiment 199 or 200, wherein modulating an immune response in a subject comprises at least one selected from the group consisting of activating γδ T cells, causing or increasing proliferation γδ T cells, causing or increasing expansion of γδ T cells, causing or increasing γδ T cell degranulation, causing or increasing γδ T cell mediated killing activity, causing or increasing γδ T cytotoxicity, causing or increasing γδ T cell mobilization, increasing survival of γδ T cells, and increasing resistance to exhaustion of γδ T cells.
202. The method of any one of embodiments 194 to 201, wherein diseased cells are killed while healthy cells are spared.
203. The multispecific antibody of any one of embodiments 1 to 178, or the pharmaceutical composition of embodiment 190, or the kit of any one of embodiments 191 to 193 for use in medicine.
204. The multispecific antibody or antigen-binding fragment thereof of any one of embodiments 1 to 178, or the pharmaceutical composition of embodiment 190, or the kit of any one of embodiments 191 to 193 for use in the treatment of cancer.
205. Use of a multispecific antibody or antigen-binding fragment thereof of any one of embodiments 1 to 178 in the manufacture of a medicament.
206. Use of a multispecific antibody or antigen-binding fragment thereof of any one of embodiments 1 to 178 in the manufacture of a medicament for the treatment of cancer.
207. The method or use of embodiment 206, wherein the cancer is a PD-L1 positive cancer.
208. The method or use of embodiment 207, wherein the cancer is selected from the group consisting of head and neck squamous cell carcinoma (HNSCC), non-small cell lung cancer (NSCLC), colorectal cancer (CRC) and gastric cancers.
209. The multispecific antibody as described in any previous embodiment, wherein the multispecific antibody is a Vδ1 agonist.
210. The multispecific antibody as described in any previous embodiment, wherein the multispecific antibody is a PD-L1 antagonist.
211. The multispecific antibody as described in any previous embodiment, wherein the multispecific antibody is a Vδ1 agonist and a PD-L1 antagonist.

1.-38. (canceled)

39. A multispecific antibody or antigen-binding fragment thereof comprising:
(i) a variable delta 1 (Vδ1) chain of a 76 T cell receptor (TCR) binding Fab region comprising:
a variable heavy chain complementary determining region 1 (VLCDR1), a variable heavy chain complementary determining region 2 (VHCDR2) and a variable heavy chain complementary determining region 3 (VHCDR3) comprising the amino acid sequences of SEQ ID NO: 51, 53 and 68, respectively; and
a variable light chain complementary determining region 1 (VLCDR1), a variable light chain complementary determining region 2 (VLCDR2), and a variable light chain complementary determining region 3 (VLCDR3) comprising the amino acid sequences of SEQ ID NOs: 79, 80 and 95, respectively; and
(ii) a PD-L1 binding Fc region comprising a CH3 domain that comprises an AB structural loop comprising the amino acid sequence of SEQ ID NO: 565 or 569, and an EF structural loop comprising the amino acid sequence of SEQ ID NO: 567.

40. The multispecific antibody or antigen-binding fragment thereof of claim 39, wherein the AB structural loop is at positions 359 to 362 of the CH3 domain and the EF structural loop is at positions 413 to 421 of the CH3 domain, wherein the amino acid residue numbering of the CH3 domain is according to the EU numbering scheme.

41. The multispecific antibody or antigen-binding fragment thereof of claim 39, wherein the PD-L1 binding Fc CH3 region comprises a CD loop of SEQ ID NO: 566.

42. The multispecific antibody or antigen-binding fragment thereof of claim 39, wherein the AB structural loop comprises the amino acid sequence of SEQ ID NO: 565.

43. The multispecific antibody or antigen-binding fragment thereof of claim 39, wherein AB structural loop comprises the amino acid sequence of SEQ ID NO: 569.

44. The multispecific antibody or antigen-binding fragment thereof of claim 39, wherein the PD-L1 binding Fc CH3 region comprises an amino acid sequence of SEQ ID NO: 564.

45. The multispecific antibody or antigen-binding fragment thereof of claim 39, wherein the PD-L1 binding Fc CH3 region comprises an amino acid sequence of SEQ ID NO: 568.

46. The multispecific antibody or antigen-binding fragment thereof of claim 39, wherein the Vδ1 TCR binding Fab region comprises a variable heavy chain (VH) of SEQ ID NO: 15 and a variable light chain (VL) of SEQ ID NO: 40.

47. The multispecific antibody or antigen-binding fragment thereof of claim 46, comprising wherein the Vδ1 TCR binding Fab region comprises an amino acid sequence of SEQ ID NO: X2.

48. The multispecific antibody of claim 39, wherein:
the Vδ1 TCR binding Fab region comprises a variable heavy chain (VH) of SEQ ID NO: 15 and a variable light chain (VL) of SEQ ID NO: 40; and
the PD-L1 binding Fc CH3 region comprises an amino acid sequence of SEQ ID NO: 564.

49. The multispecific antibody of claim 39, wherein:
the Vδ1 TCR binding Fab region comprises a variable heavy chain (VH) of SEQ ID NO: 15 and a variable light chain (VL) of SEQ ID NO: 40; and
the PD-L1 binding Fc CH3 region comprises an amino acid sequence of SEQ ID NO: 568.

50. The multispecific antibody of claim 39, wherein the PD-L1 binding Fc comprises an amino acid sequence of SEQ ID NO: 585 or 586.

51. The multispecific antibody of claim 39, wherein the PD-L1 binding Fc comprises an amino acid sequence of SEQ ID NO: 595 or 596.

52. A bispecific antibody comprising the amino acid sequence of SEQ ID NO: 414 and the amino acid sequence of SEQ ID NO: 587.

53. A bispecific antibody comprising the amino acid sequence of SEQ ID NO: 414 and the amino acid sequence of SEQ ID NO: 597.

54. A method of treating a cancer in a subject, the method comprising administering a multispecific antibody of claim 39 to the subject.

55. The method of claim 54, wherein the cancer is head and neck squamous cell carcinoma (HNSCC), non-small cell lung cancer (NSCLC), colorectal cancer (CRC) or gastric cancer.

56. The method of claim 54, wherein:
the Vδ1 TCR binding Fab region comprises a variable heavy chain (VH) of SEQ ID NO: 15 and a variable light chain (VL) of SEQ ID NO: 40; and
the PD-L1 binding Fc CH3 region comprises the amino acid sequence of SEQ ID NO: 564.

57. The method of claim 54, wherein:
the Vδ1 TCR binding Fab region comprises a variable heavy chain (VH) of SEQ ID NO: 15 and a variable light chain (VL) of SEQ ID NO: 40; and
the PD-L1 binding Fc CH3 region comprises the amino acid sequence of SEQ ID NO: 568.

58. The method of claim 54, wherein the multispecific antibody comprises:
the amino acid sequence of SEQ ID NO: 414 and the amino acid sequence of SEQ ID NO: 587; or
the amino acid sequence of SEQ ID NO: 414 and the amino acid sequence of SEQ ID NO: 597.

59. A pharmaceutical composition comprising:
the multispecific antibody of claim 39; and
a pharmaceutically acceptable excipient.
